(19) United States
(12) Patent Application Publication
(10) Pub. No.: US 2023/0151070 A1
(43) Pub. Date: May 18, 2023

(54) VEGFR-3-ACTIVATING AGENTS AND ONCOLYTIC VIRUSES AND USES THEREOF FOR THE TREATMENT OF CANCER

(71) Applicant: ICAHN SCHOOL OF MEDICINE AT MOUNT SINAI, New York, NY (US)

(72) Inventors: Mihaela SKOBE, New York, NY (US); Andrew Kenneth EDWARDS, New York, NY (US); Ruben FERNANDEZ-RODRIGUEZ, New York, NY (US); Adolfo GARCIA-SASTRE, New York, NY (US); Ignacio MENA, New York, NY (US); Sara CUADRADO CASTAÑO, New York, NY (US)

(73) Assignee: ICAHN SCHOOL OF MEDICINE AT MOUNT SINAI, New York, NY (US)

(21) Appl. No.: 17/802,789

(22) PCT Filed: Feb. 26, 2021
(86) PCT No.: PCT/US2021/020085

§ 371 Date: Aug. 26, 2022

Related U.S. Application Data
(60) Provisional application No. 62/982,648, filed on Feb. 27, 2020.
(60) Provisional application No. 62/982,651, filed on Feb. 27, 2020.

(51) Int. Cl. C07K 14/475; A61P 35/00; C12N 15/86

(52) U.S. Cl. CPC C07K 14/475; A61P 35/00; C12N 15/86; C12N 2760/18143

(57) ABSTRACT

In one aspect, provided herein are methods for treating cancer in a subject, comprising administering to a subject an oncolytic virus (e.g., an avian paramyxovirus (AMPV)) and a vascular endothelial growth factor (VEGF)-C agent, a VEGF-D agent, or a VEGF receptor (VEGFR)-3-activating agent. In another aspect, provided herein are oncolytic viruses (e.g. APMV) comprising a genome, wherein the genome comprises a transgene that comprises a nucleotide sequence encoding a VEGF-C agent, a VEGF-D agent or a VEGF receptor (VEGFR)-3-activating agent. In another aspect, provided herein are methods for treating cancer, comprising administering to a subject an oncolytic virus (e.g. APMV), wherein the oncolytic virus comprises a genome that comprises a transgene comprising a nucleotide sequence encoding a VEGF-C agent, a VEGF-D agent, or a VEGF receptor (VEGFR)-3-activating agent.

### CROSS REFERENCE TO RELATED APPLICATIONS

This application claims the benefit of U.S. Provisional Patent Application No. 62/982,648, filed Feb. 27, 2020 and U.S. Provisional Patent Application No. 62/982,651, filed Feb. 27, 2020, each of which is incorporated by reference herein in its entirety.

### REFERENCE TO SEQUENCE LISTING FILED ELECTRONICALLY

This application incorporates by reference a Sequence Listing submitted with this application as text file entitled “06923-307-228_SEQ_LISTING.txt” created on Feb. 25, 2021 and having a size of 365,513 bytes.

### 1. INTRODUCTION

In one aspect, provided herein are methods for treating cancer in a subject, comprising administering to a subject an oncolytic virus (e.g., an avian paramyxovirus (AMPV)) and a vascular endothelial growth factor (VEGF)-C agent, a VEGF-D agent, or another VEGF receptor (VEGFR)-3-activating agent. In another aspect, provided herein are oncolytic viruses (e.g. APMV) comprising a genome, wherein the genome comprises a transgene that comprises a nucleotide sequence encoding a VEGF-C agent, a VEGF-D agent or another VEGF receptor (VEGFR)-3-activating agent. In another aspect, provided herein are methods for treating cancer, comprising administering to a subject an oncolytic virus (e.g. APMV), wherein the oncolytic virus comprises a genome that comprises a transgene comprising a nucleotide sequence encoding a VEGF-C agent, a VEGF-D agent, or another VEGF receptor (VEGFR)-3-activating agent.

### 2. BACKGROUND

VEGF-C belongs to the VEGF family, which also includes VEGF-A, placental growth factor, VEGF-B, and VEGF-D. VEGF-C is a ligand for the Flt4 (VEGFR-3) and KDR (VEGFR-2) receptor tyrosine kinases (Joukov V, Kumar V, Sorsa T, Arighi E, Weich H, Saksela O, Alitalo K (1998) A recombinant mutant vascular endothelial growth factor-C that has lost vascular endothelial growth factor receptor-2 binding, activation, and vascular permeability activities J Biol Chem 273:6599-6602). VEGF-D is closely related to VEGF-C; VEGF-D is structurally and functionally similar to VEGF-C (Achen et al., 1998, PNAS 95(2): 548-553). Like VEGF-C, VEGF-D is a ligand for VEGFR-2 and VEGFR-3 (id.). Lymphangiogenesis—the growth of lymphatic vessels from pre-existing ones—occurs mainly in response to VEGF-C and VEGF-D induced VEGFR3 activation (Jeltsch Metal. (1997) Hyperplasia of lymphatic vessels in VEGF-C transgenic mice Science 276:1423-1425; Karkkainen M J et al. (2004) Vascular endothelial growth factor C is required for sprouting of the first lymphatic vessels from embryonic veins Nat Immunol 5:74-80 doi:10.1038/ni1013). VEGF-C is synthesized as a precursor in which the central VEGF homology domain (VHD) is flanked by N- and C-terminal propeptides. VEGF-C precursor undergoes proteolytic processing that generates an intermediately processed form which selectively binds VEGFR-3 and fully processed (mature) form that has increased affinity for VEGFR-3 and also binds the major angiogenic receptor VEGFR2 (Bui H M et al. (2016) Proteolytic activation defines distinct lymphangiogenic mechanisms for VEGFC and VEGFD J Clin Invest 126:2167-2180 doi:10.1172/JCI83967; Jeltsch M et al. (2014) CCBE1 enhances lymphangiogenesis via A disintegrin and metalloprotease with thrombospondin motifs-3-mediated vascular endothelial growth factor-C activation Circulation 129:1962-1971 doi:10.1161/CIRCULATIONAHA.113.002779; Joukov et al. 1997; Le Guen L et al. (2014) Ccbel regulates Vegfc-mediated induction of Vegfr3 signaling during embryonic lymphangiogenesis Development 141:1239-1249 doi:10.1242/dev.100495; Roukens M G et al. (2015) Functional Dissection of the CCBE1 Protein: A Crucial Requirement for the Collagen Repeat Domain Circ Res 116:1660-1669 doi:10.1161/CIRCRESAHA.116.304949). VEGF-D also undergoes proteolytic processing, which is necessary for producing active, mature form of VEGF-D. However, proteolytic cleavage of VEGF-D involves different proteases than that of VEGF-C (McColl B K et al. (2003) Plasmin activates the lymphangiogenic growth factors VEGF-C and VEGF-D J Exp Med 198:863-868 doi:10.1084/jem.20030361).

VEGFR-3 is primarily expressed by lymphatic endothelial cells. It is phosphorylated following activation with its ligands VEGF-C and VEGF-D, leading to downstream signaling events. In particular, VEGF-C-induced VEGFR-3 activation leads to phosphorylation of the serine/threonine kinases AKT and ERK, which promote lymphatic endothelial cell (LEC) proliferation, migration and survival (Gibot L, Galbraith T, Kloos B, Das S, Lacroix D A, Auger F A, Skobe M (2016) Cell-based approach for 3D reconstruction of lymphatic capillaries in vitro reveals distinct functions of HGF and VEGF-C in lymphangiogenesis Biomaterials 78:129-139 doi:10.1016/j.biomaterials.2015.11.027; Makinen T et al. (2001) Isolated lymphatic endothelial cells transduce growth, survival and migratory signals via the VEGF-C/D receptor VEGFR-3 EMBO J 20:4762-4773 doi:10.1093/emboj/20.17.4762; Salameh A, Galvagni F, Bardelli M, Bussolino F, Oliviero S (2005) Direct recruitment of CRK and GRB2 to VEGFR-3 induces proliferation, migration, and survival of endothelial cells through the activation of ERK, AKT, and JNK pathways Blood 106:3423-3431 doi:10.1182/blood-2005-04-1388).

VEGF-C is a key lymphangiogenesis factor. Thus far, VEGF-C has been considered as a therapeutic modality for lymphedema patients, to promote regeneration of new lymphatic vessels (Baker, A., Kim, H., Semple, J. L., Dumont, D., Shoichet, M., Tobbia, D., and Johnston, M. (2010). Experimental assessment of pro-lymphangiogenic growth factors in the treatment of post-surgical lymphedema following lymphadenectomy. Breast Cancer Res 12, R70; Szuba, A., Skobe, M., Karkkainen, M. J., Shin, W. S., Beynet, D. P., Rockson, N. B., Dakhil, N., Spilman, S., Goris, M. L., Strauss, H. W., et al. (2002). Therapeutic lymphangiogenesis with human recombinant VEGF-C. FASEB J 16, 1985-1987.; Visuri, M. T., Honkonen, K. M., Hartiala, P., Tervala, T. V., Halonen, P. J., Junkkari, H., Knuutinen, N., Yla-Herttuala, S., Alitalo, K. K., and Saarikko, A. M. (2015). VEGF-C and VEGF-C156S in the pro-lymphangiogenic growth factor therapy of lymphedema: a large animal study. Angiogenesis 18, 313-326.; Yoon, Y. S., Murayama, T., Gravereaux, E., Tkebuchava, T., Silver, M., Curry, C., Wecker, A., Kirchmair, R., Hu, C. S., Kearney, M., et al. (2003). VEGF-C gene therapy augments postnatal lymphangiogenesis and ameliorates secondary lymphedema. J Clin Invest 111, 717-725.). Inhibition of VEGF-C or its receptor VEGFR-3 has been evaluated as a therapeutic approach for preventing and inhibiting metastasis, because VEGF-C mediated induction of lymphangiogenesis promotes metastasis in many cancer types (Burton et al., 2008; Das et al., 2010; Lin et al., 2005; Roberts et al., 2006; Saif et al., 2016; Skobe et al., 2001; Stacker et al., 2014).

Cancer is a second leading cause of death worldwide, the most common cancers being lung cancer, breast cancer, colorectal cancer, prostate cancer, skin cancer and stomach cancer. See, World Health Organization Fact Sheet Cancer, September 2018, available at: https://www.who.int/news-room/fact-sheets/detail/cancer (accessed Feb. 11, 2020). Existing therapies to treat cancer are often limited in their application due to variable efficacy between patients and high toxicity. See Voon and Kong, 2011, “Tumour Genetics and Genomics to Personalise Cancer Treatment”, Ann Acad Med Singapore 2011; 40:362-8. Thus, effective therapies for treating cancer are needed.

### 3. SUMMARY

In one aspect, provided herein are recombinant nucleic acid sequences comprising a nucleotide sequence of an oncolytic virus genome and a transgene, wherein the transgene comprises a nucleotide sequence encoding a nucleotide sequence encoding a VEGFR-3 activating agent. See section 5.2 and 5.3.2 for examples of VEGFR-3 activating agents. In another aspect, provided herein are recombinant nucleic acid sequences comprising a nucleotide sequence of an oncolytic virus genome and a transgene, wherein the transgene comprises a nucleotide sequence encoding vascular endothelial growth factor (VEGF)-C or VEGF-D. See sections 5.2 and 5.3.2 for examples of VEGF-C and VEGF-D sequences. In certain embodiments, the nucleotide sequence encoding VEGF-C comprises the sequence set forth in any one of SEQ ID NOs: 1-18, 29-40, 49, or 50. In some embodiments, the VEGF-C comprises the amino acid sequence set forth in any one of SEQ ID NOs: 19-24, 41-46, 51, or 52. In certain embodiments, the nucleotide sequence encoding VEGF-D comprises the sequence set forth in any one of SEQ ID NOs: 96-98. In some embodiments, the VEGF-D comprises the amino acid sequence set forth in any one of SEQ ID NO: 99-104. See, e.g., Section 5.1 and 5.3 for examples of oncolytic viruses. In one embodiment, the oncolytic virus is a parvovirus, a myxoma virus, a Newcastle disease virus, an APMV-2, an APMV-3, an APMV-4, an APMV-5, an APMV-6, an APMV-7, an APMV-8, or an APMV-9, a reovirus, or Seneca valley virus. In another embodiment, the oncolytic virus is a genetically engineered influenza virus, measles virus, poliovirus, vaccinia virus, poxvirus, picornavirus, alphavirus, retrovirus, rhabdovirus, reovirus, adenovirus, herpes simplex virus, or vesicular stomatitis virus.

In another aspect, provided herein are recombinant nucleic acid sequences comprising a nucleotide sequence of an avian paramyxovirus (APMV) genome and a transgene, wherein the transgene comprises a nucleotide sequence encoding a VEGFR-3 activating agent. In another aspect, provided herein is a recombinant nucleic acid sequence comprising a nucleotide sequence of an avian paramyxovirus (APMV) genome and a transgene, wherein the transgene comprises a nucleotide sequence encoding vascular endothelial growth factor (VEGF)-C or VEGF-D. See sections 5.2 and 5.3.2 for examples of VEGF-C and VEGF-D sequences. In a specific embodiment, the genome comprises a transcription unit encoding a nucleocapsid (N) protein, a transcription unit encoding a phosphoprotein (P), a transcription unit encoding a matrix (M) protein, a transcription unit encoding a fusion (F) protein, a transcription unit encoding a hemagglutinin-neuraminidase (HN), and a transcription unit encoding a large polymerase (L) protein. In another specific embodiment, the transgene is incorporated between the M and P transcription units or between the HN and L transcription units. In a particular embodiment, the APMV is Newcastle disease virus (NDV). In another embodiment, the APMV is NDV and the F protein of the NDV contains a leucine to alanine substitution at amino acid residue 289. In a specific embodiment, the APMV is NDV and the transgene comprises the nucleotide sequence of SEQ ID NO: 87. In another embodiment, the APMV is APMV serotype 4 (APMV-4). In a specific embodiment, APMV is AMPV-4 and the transgene comprises the nucleotide sequence of SEQ ID NO: 89. In certain embodiments, the nucleotide sequence encoding VEGF-C comprises the sequence set forth in any one of SEQ ID NOs: 1-18, 29-40, 49, or 50. In a specific embodiment, the nucleotide sequence encoding VEGF-C comprises the sequence set forth in any one of SEQ ID Nos: 29-40. In some embodiments, the VEGF-C comprises the amino acid sequence set forth in any one of SEQ ID NOs: 19-24, 41-46, 51, or 52. In a specific embodiment, the VEGF-C comprises the amino acid sequence set forth in any one of SEQ ID Nos: 41-46. In certain embodiments, the nucleotide sequence encoding VEGF-D comprises the sequence set forth in any one of SEQ ID NOs: 96-98. In some embodiments, the VEGF-D comprises the amino acid sequence set forth in any one of SEQ ID NOs: 99-104. In a specific embodiment, provided herein is a recombinant nucleic acid sequence comprising the nucleotide sequence of SEQ ID NO: 88 or 90.

In another aspect, provided herein is a recombinant oncolytic virus comprising a genome that comprises a transgene, wherein the transgene comprises a nucleotide sequence encoding a VEGFR-3 activating agent. In another aspect, provided herein is a recombinant oncolytic virus comprising a genome that comprises a transgene, wherein the transgene comprises a nucleotide sequence encoding VEGF-C or VEGF-D. See, e.g., Section 5.1 and 5.3 for examples of oncolytic viruses. In one embodiment, the oncolytic virus is a parvovirus, a myxoma virus, a Newcastle disease virus, an APMV-2, an APMV-3, an APMV-4, an APMV-5, an APMV-6, an APMV-7, an APMV-8, or an APMV-9, a reovirus, or Seneca valley virus. In another embodiment, the oncolytic virus is a genetically engineered influenza virus, measles virus, poliovirus, vaccinia virus, poxvirus, picornavirus, alphavirus, retrovirus, rhabdovirus, reovirus, adenovirus, herpes simplex virus, or vesicular stomatitis virus. In some embodiments, the nucleotide sequence encoding VEGF-C comprises the sequence set forth in any one of SEQ ID NOs: 1-18, 29-40, 49, or 50. In a specific embodiment, the VEGF-C comprises the amino acid sequence set forth in any one of SEQ ID Nos: 29-40. In certain embodiments, the VEGF-C comprises the amino acid sequence set forth in any one of SEQ ID NOs: 19-24, 41-46, 51, or 52. In a specific embodiment, the VEGF-C comprises the amino acid sequence set forth in any one of SEQ ID Nos: 41-46. In some embodiments, the nucleotide sequence encoding VEGF-D comprises the sequence set forth in any one of SEQ ID NOs: 96-98. In certain embodiments, the VEGF-D comprises the amino acid sequence set forth in any one of SEQ ID NOs: 99-104.

In another aspect, provided herein is a recombinant avian paramyxovirus (APMV) comprising a packaged genome, wherein the packaged genome comprises a transgene that comprises a nucleotide sequence encoding VEGF-C or VEGF-D. In a specific embodiment, the genome comprises a transcription unit encoding a nucleocapsid (N) protein, a transcription unit encoding a phosphoprotein (P), a transcription unit encoding a matrix (M) protein, a transcription unit encoding a fusion (F) protein, a transcription unit encoding a hemagglutinin-neuraminidase (HN), and a transcription unit encoding a large polymerase (L) protein. In another specific embodiment, the transgene is incorporated between the M and P transcription units or between the HN and L transcription units. In a particular embodiment, the APMV is Newcastle disease virus (NDV). In another embodiment, the APMV is NDV and the F protein of the NDV contains a leucine to alanine substitution at amino acid residue 289. In a specific embodiment, the APMV is NDV and the transgene comprises the nucleotide sequence of SEQ ID NO: 87. In another embodiment, the APMV is APMV serotype 4 (APMV-4). In a specific embodiment, APMV is AMPV-4 and the transgene comprises the nucleotide sequence of SEQ ID NO: 89. In certain embodiments, the nucleotide sequence encoding VEGF-C comprises the sequence set forth in any one of SEQ ID Nos: 1-18, 29-40, 49, or 50. In a specific embodiment, the VEGF-C comprises the amino acid sequence set forth in any one of SEQ ID Nos: 29-40. In some embodiments, the VEGF-C comprises the amino acid sequence set forth in any one of SEQ ID NOs: 19-24, 41-46, 51, or 52. In a specific embodiment, the VEGF-C comprises the amino acid sequence set forth in any one of SEQ ID Nos: 41-46. In certain embodiments, the nucleotide sequence encoding VEGF-D comprises the sequence set forth in any one of SEQ ID NOs: 96-98. In some embodiments, the VEGF-D comprises the amino acid sequence set forth in any one of SEQ ID NO: 99-104. In a specific embodiment, provided herein is a recombinant nucleic acid sequence comprising the nucleotide sequence of SEQ ID NO: 88 or 90.

In another aspect, provided herein are pharmaceutical compositions comprising an oncolytic virus described herein (e.g., an APMV described herein) in a pharmaceutically acceptable carrier or excipient. In certain embodiments, a pharmaceutical composition contains 10⁶ to 10¹⁰ plaque forming units (pfu) of an oncolytic virus described herein. In a specific embodiment, provided herein is a pharmaceutical composition comprising a recombinant APMV described herein in a pharmaceutically acceptable carrier or excipient.

In another aspect, provided herein are methods for treating cancer comprising administering an oncolytic virus described herein or a composition thereof to a subject. In a specific embodiment, provided herein is a method for treating cancer, comprising administering a dose of a pharmaceutical composition described herein to a subject. In some embodiments, a therapeutically-effective dose of the pharmaceutical composition is administered. In some embodiments, the oncolytic virus or pharmaceutical composition is administered to the subject intratumorally. In certain embodiments, a dose of a pharmaceutical composition contains 10⁶ to 10¹⁰ plaque forming units (pfu) of an oncolytic virus described herein. In specific embodiments, the cancer treated in accordance with the methods described herein is melanoma, lung carcinoma, colon carcinoma, glioblastoma, head and neck cancer, pancreatic cancer, hepatocellular carcinoma, ovarian cancer, squamous cell cancer, basal cell cancer, bladder cancer, prostate cancer, B-cell lymphoma, T-cell lymphoma, gastric cancer, colorectal cancer or breast cancer. In certain embodiments, the cancer treated in accordance with the methods described herein is metastatic. In some embodiments, the cancer treated in accordance with the methods described herein is unresectable. In a specific embodiment, the subject treated in accordance with the methods described herein is human.

In another aspect, provided herein are methods for treating cancer, comprising administering to a subject an oncolytic virus or a composition thereof, and administering a VEGFR3 activating agent or a composition thereof. The activating agent may be in the same or different compositions as the oncolytic virus. See section 5.2 and 5.3.2 for examples of VEGFR-3-activating agents. In another aspect, provided are methods for treating cancer, comprising administering to a subject an oncolytic virus and administering to the subject VEGF-C or VEGF-D. In some embodiments, the oncolytic virus and the VEGF-C or VEGF-D are in the same composition. In other embodiments, the oncolytic virus and the VEGF-C or VEGF-D are in different compositions. The different compositions may be administered to the subject concurrently or at different times. The oncolytic virus may be administered to the subject intratumorally and the VEGF-C or VEGF-D may be administered to the subject intratumorally, intramuscularly, intranasally, intradermally or subcutaneously. In a specific embodiment, provided herein are methods for treating cancer, comprising administering intratumorally to a subject a dose of a first pharmaceutical composition comprising an oncolytic virus and administering to the subject a dose of a second pharmaceutical composition comprising VEGF-C or VEGF-D. In some embodiments, a therapeutically-effective dose of the first pharmaceutical composition, a therapeutically-effective dose the second pharmaceutical composition, or both is administered to the subject. In certain embodiments, the VEGF-C is encoded by a nucleotide sequence comprising the sequence set forth in any one of SEQ ID NOs: 1-18, 29-40, 49, or 50. In a specific embodiment, the VEGF-C comprises the amino acid sequence set forth in any one of SEQ ID Nos: 29-40. In some embodiments, the VEGF-C comprises the amino acid sequence set forth in any one of SEQ ID NOs: 19-24, 41-46, 51, or 52. In a specific embodiment, the VEGF-C comprises the amino acid sequence set forth in any one of SEQ ID Nos: 41-46. In certain embodiments, the VEGF-D is encoded by a nucleotide sequence comprising the sequence set forth in any one of SEQ ID NOs: 96-98. In some embodiments, the VEGF-D comprises the amino acid sequence set forth in any one of SEQ ID NOs: 99-104. In certain embodiments, the second pharmaceutical composition is administered to the subject intratumorally, intramuscularly, intranasally, intradermally, or subcutaneously. In some embodiments, the subject treated in accordance with the methods described herein is not administered an antigen (e.g., a cancer antigen). In certain embodiments, a dose of the first pharmaceutical composition contains 10⁶ to 10¹⁰ pfu of the virus. See, e.g., Section 5.1 and 5.3 for examples of oncolytic viruses. In one embodiment, the oncolytic virus is a parvovirus, a myxoma virus, a Newcastle disease virus, an APMV-2, an APMV-3, an APMV-4, an APMV-5, an APMV-6, an APMV-7, an APMV-8, or an APMV-9, a reovirus, or Seneca valley virus. In another embodiment, the oncolytic virus is a genetically engineered influenza virus, measles virus, poliovirus, vaccinia virus, poxvirus, picornavirus, alphavirus, retrovirus, rhabdovirus, reovirus, adenovirus, herpes simplex virus, or vesicular stomatitis virus. In some embodiments, the oncolytic virus is an APMV (e.g., APMV-4 or Newcastle disease virus). In specific embodiments, the cancer treated in accordance with the methods described herein is melanoma, lung carcinoma, colon carcinoma, glioblastoma, head and neck cancer, pancreatic cancer, hepatocellular carcinoma, ovarian cancer, squamous cell cancer, basal cell cancer, bladder cancer, prostate cancer, B-cell lymphoma, T-cell lymphoma, gastric cancer, colorectal cancer or breast cancer. In certain embodiments, the cancer treated in accordance with the methods described herein is metastatic. In some embodiments, the cancer treated in accordance with the methods described herein is unresectable. In a specific embodiment, the subject treated in accordance with the methods described herein is human.

In another aspect, provided are methods for treating cancer, comprising administering to a subject an oncolytic virus and administering to the subject a nucleic acid sequence comprising a nucleotide sequence encoding VEGF-C or VEGF-D. In some embodiments, the oncolytic virus and the nucleotide sequence are in the same composition. In other embodiments, the oncolytic virus and the nucleotide sequence are in different compositions. The different compositions may be administered to the subject concurrently or at different times. The oncolytic virus may be administered to the subject intratumorally and the nucleotide sequence may be administered to the subject intratumorally, intramuscularly, intranasally, intradermally or subcutaneously. In a specific embodiment, provided herein is a method for treating cancer, comprising administering intratumorally to a subject a dose of a first pharmaceutical composition comprising an oncolytic virus and administering to the subject a dose of a second pharmaceutical composition a nucleic acid sequence comprising a nucleotide sequence encoding VEGF-C or VEGF-D. In some embodiments, a therapeutically-effective dose of the first pharmaceutical composition, a therapeutically-effective dose of the second pharmaceutical composition, or both is administered to the subject. In certain embodiments, the nucleotide sequence encoding VEGF-C comprises the sequence set forth in any one of SEQ ID NOs: 1-18, 29-40, 49, or 50. In a specific embodiment, the VEGF-C comprises the amino acid sequence set forth in any one of SEQ ID Nos: 29-40. In some embodiments, the VEGF-C comprises the amino acid sequence set forth in any one of SEQ ID NOs: 19-24, 41-46, 51, or 52. In a specific embodiment, the VEGF-C comprises the amino acid sequence set forth in any one of SEQ ID Nos: 41-46. In certain embodiments, the nucleotide sequence encoding VEGF-D comprises the sequence set forth in any one of SEQ ID NOs: 96-98. In some embodiments, the VEGF-D comprises the amino acid sequence set forth in any one of SEQ ID NO: 99-104. In certain embodiments, the second pharmaceutical composition is administered to the subject intratumorally, intramuscularly, intranasally, intradermally or subcutaneously. In some embodiments, the subject treated in accordance with the methods described herein is not administered an antigen (e.g., a cancer antigen). In certain embodiments, a dose of the first pharmaceutical composition contains 10⁶ to 10¹⁰ pfu of the virus. See, e.g., Section 5.1 and 5.3 for examples of oncolytic viruses. In one embodiment, the oncolytic virus is a parvovirus, a myxoma virus, a Newcastle disease virus, an APMV-2, an APMV-3, an APMV-4, an APMV-5, an APMV-6, an APMV-7, an APMV-8, or an APMV-9, a reovirus, or Seneca valley virus. In another embodiment, the oncolytic virus is a genetically engineered influenza virus, measles virus, poliovirus, vaccinia virus, poxvirus, picornavirus, alphavirus, retrovirus, rhabdovirus, reovirus, adenovirus, herpes simplex virus, or vesicular stomatitis virus. In some embodiments, the oncolytic virus is an APMV (e.g., APMV-4 or Newcastle disease virus). In specific embodiments, the cancer treated in accordance with the methods described herein is melanoma, lung carcinoma, colon carcinoma, glioblastoma, head and neck cancer, pancreatic cancer, hepatocellular carcinoma, ovarian cancer, squamous cell cancer, basal cell cancer, bladder cancer, prostate cancer, B-cell lymphoma, T-cell lymphoma, gastric cancer, colorectal cancer or breast cancer. In certain embodiments, the cancer treated in accordance with the methods described herein is metastatic. In some embodiments, the cancer treated in accordance with the methods described herein is unresectable. In a specific embodiment, the subject treated in accordance with the methods described herein is human.

In a specific embodiment, provided herein is a method for treating cancer, comprising administering a therapeutically effective dose of the pharmaceutical composition described herein to a subject (e.g., human subject) in need thereof. In certain embodiments, the pharmaceutical composition is administered to the subject intratumorally. In some embodiments, the therapeutically effective dose comprises 10⁶ to 10¹⁰ pfu of the virus. In certain embodiments, the cancer treated is melanoma, lung carcinoma, colon carcinoma, glioblastoma, head and neck cancer, pancreatic cancer, hepatocellular carcinoma, ovarian cancer, squamous cell cancer, basal cell cancer, bladder cancer, prostate cancer, B-cell lymphoma, T-cell lymphoma, or breast cancer. In some embodiments, the cancer is metastatic. In certain embodiments, the cancer is unresectable.

In another specific embodiment, provided herein is a method for treating cancer, comprising administering (e.g, intratumorally administering) to a subject (e.g., a human subject) in need thereof a therapeutically effective dose of a first pharmaceutical composition comprising an oncolytic virus (e.g., an APMV, such as APMV-1 or APMV-4) and administering to the subject a therapeutically effective dose of a second pharmaceutical composition comprising VEGF-C or VEGF-D. In another specific embodiment, provided herein is a method for treating cancer, comprising administering (e.g., intratumorally administering) to a subject (e.g., a human subject) in need thereof a dose of a first pharmaceutical composition comprising an oncolytic virus (e.g., an APMV, such as APMV-1 or APMV-4) and administering to the subject a dose of a second pharmaceutical composition comprising a nucleotide sequence encoding VEGF-C or VEGF-D. In certain embodiments, the nucleotide sequence encodes VEGF-C, and the nucleotide sequence that encodes VEGF-C comprises the sequence set forth in any one of SEQ ID NOs: 1-18, 29-40, 49, or 50. In some embodiments, the VEGF-C comprises the amino acid sequence set forth in any one of SEQ ID NOs: 19-24, 41-46, 51, or 52. In some embodiments, the nucleotide sequence encodes VEGF-D and the nucleotide sequence that encodes VEGF-D comprises the sequence set forth in any one of SEQ ID NOs: 96-98. In certain embodiments, the VEGF-D comprises the amino acid sequence set forth in any one of SEQ ID NOs: 99-104. In some embodiments, the second pharmaceutical composition is administered to the subject intratumorally, intramuscularly, intranasally, intradermally, or subcutaneously. In some embodiments, the subject is not administered an antigen. In certain embodiments, the therapeutically effective dose of the first pharmaceutical composition contains 10⁶ to 10¹⁰ pfu of the virus. In specific embodiments, the therapeutically effective dose of the VEGF-C agent or VEGF-D agent is 1 mg/kg to 100 mg/kg if the agent is proteinaceous. In certain embodiments, the cancer treated is melanoma, lung carcinoma, colon carcinoma, glioblastoma, head and neck cancer, pancreatic cancer, hepatocellular carcinoma, ovarian cancer, squamous cell cancer, basal cell cancer, bladder cancer, prostate cancer, B-cell lymphoma, T-cell lymphoma, or breast cancer. In some embodiments, the cancer is metastatic. In certain embodiments, the cancer is unresectable.

### 3.1 Terminology

As used herein, the term “about” or “approximately” when used in conjunction with a number refers to any number within 1, 5 or 10% of the referenced number, including the referenced number.

As used herein, the terms “antibody” and “antibodies” refer to molecules that contain an antigen-binding site, e.g., immunoglobulins. Antibodies include, but are not limited to, monoclonal antibodies, bispecific antibodies, multispecific antibodies, human antibodies, humanized antibodies, synthetic antibodies, chimeric antibodies, polyclonal antibodies, single domain antibodies, camelized antibodies, single-chain Fvs (scFv), single chain antibodies, Fab fragments, F(ab′) fragments, disulfide-linked bispecific Fvs (sdFv), intrabodies, and anti-idiotypic (anti-Id) antibodies (including, e.g., anti-Id and anti-anti-Id antibodies to antibodies), and epitope-binding fragments of any of the above. In particular, antibodies include immunoglobulin molecules and immunologically active fragments of immunoglobulin molecules. Immunoglobulin molecules can be of any type (e.g., IgG, IgE, IgM, IgD, IgA and IgY), class (e.g., IgG1, IgG2, IgG3, IgG4, IgA1 and IgA2) or subclass. In a specific embodiment, an antibody is a human or humanized antibody. In another specific embodiment, an antibody is a monoclonal antibody or scFv. In certain embodiments, an antibody is a human or humanized monoclonal antibody or scFv. In other specific embodiments, the antibody is a bispecific antibody.

As used herein, the term “elderly human” refers to a human 65 years or older.

As used herein, the term “fragment” in the context of a nucleotide sequence refers to a nucleotide sequence comprising a nucleic acid sequence of at least 5 contiguous nucleic acid bases, at least 10 contiguous nucleic acid bases, at least 15 contiguous nucleic acid bases, at least 20 contiguous nucleic acid bases, at least 25 contiguous nucleic acid bases, at least 40 contiguous nucleic acid bases, at least 50 contiguous nucleic acid bases, at least 60 contiguous nucleic acid bases, at least 70 contiguous nucleic acid bases, at least 80 contiguous nucleic acid bases, at least 90 contiguous nucleic acid bases, at least 100 contiguous nucleic acid bases, at least 125 contiguous nucleic acid bases, at least 150 contiguous nucleic acid bases, at least 175 contiguous nucleic acid bases, at least 200 contiguous nucleic acid bases, or at least 250 contiguous nucleic acid bases of the nucleotide sequence of the gene of interest or longer nucleic acid sequence of interest. The nucleic acid may be RNA, DNA, or a chemically modified variant thereof.

As used herein, the term “fragment” is the context of a fragment of a proteinaceous agent (e.g., a protein or polypeptide) refers to a fragment that is composed of 8 or more contiguous amino acids, 10 or more contiguous amino acids, 15 or more contiguous amino acids, 20 or more contiguous amino acids, 25 or more contiguous amino acids, 50 or more contiguous amino acids, 75 or more contiguous amino acids, 100 or more contiguous amino acids, 150 or more contiguous amino acids, 200 or more contiguous amino acids, 10 to 150 contiguous amino acids, 10 to 200 contiguous amino acids, 10 to 250 contiguous amino acids, 10 to 300 contiguous amino acids, 50 to 100 contiguous amino acids, 50 to 150 contiguous amino acids, 50 to 200 contiguous amino acids, 50 to 250 contiguous amino acids or 50 to 300 contiguous amino acids of a proteinaceous agent.

As used herein, the term “heterologous” in the context of a virus to refers an entity not found in nature to be associated with (e.g., encoded by, expressed by the genome of, or both) a naturally occurring virus (e.g., a naturally occurring APMV). In a specific embodiment, a heterologous sequence in the context of a virus encodes a protein that is not found associated with naturally occurring virus (e.g., a naturally occurring APMV).

As used herein, the term “heterologous” in the context of a sequence to refers a sequence not found in nature to be associated with or part of a naturally occurring sequence.

As used herein, the term “human adult” refers to a human that is 18 years or older.

As used herein, the term “human child” refers to a human that is 1 year to 18 years old.

As used herein, the term “human infant” refers to a newborn to 1-year-old year human.

As used herein, the term “human toddler” refers to a human that is 1 year to 3 years old.

As used herein, the term “in combination” in the context of the administration of (a) therapy(ies) to a subject, refers to the use of more than one therapy. The use of the term “in combination” does not restrict the order in which therapies are administered to a subject. A first therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject. For example, a recombinant APMV described herein may be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before) concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of another therapy.

As used herein, the phrases “interferon-deficient systems,” “interferon-deficient substrates,” “IFN deficient systems” or “IFN-deficient substrates” refer to systems, e.g., cells, cell lines and animals, such as mice, chickens, turkeys, rabbits, rats, horses etc., which do not produce one, two or more types of IFN, or do not produce any type of IFN, or produce low levels of one, two or more types of IFN, or produce low levels of any IFN (i.e., a reduction in any IFN expression of 5-10%, 10-20%, 20-30%, 30-40%, 40-50%, 50-60%, 60-70%, 70-80%, 80-90% or more when compared to IFN-competent systems under the same conditions), do not respond or respond less efficiently to one, two or more types of IFN, or do not respond to any type of IFN, have a delayed response to one, two or more types of IFN, and/or are deficient in the activity of antiviral genes induced by one, two or more types of IFN, or induced by any type of IFN.

As used herein, the phrase “multiplicity of infection” or “MOI” has its customary meaning. Generally, MOI is the average number of virus per infected cell. The MOI is generally determined by dividing the number of virus added (ml added×Pfu) by the number of cells added (ml added×cells/ml).

As used herein, the term “native” in the context of proteins or polypeptides refers to any naturally occurring amino acid sequence, including immature or precursor and mature forms of a protein. In a specific embodiment, the native polypeptide is a human protein or polypeptide.

As used herein, the term “naturally occurring” in the context of a virus (e.g., an APMV) refers to a virus (e.g., an APMV) found in nature, which is not modified by the hand of man. In other words, a naturally occurring virus (e.g., a naturally occurring APMV) is not genetically engineered or otherwise altered by the hand of man.

As used herein, the terms “subject” or “patient” are used interchangeably. As used herein, the terms “subject” and “subjects” refers to an animal. In some embodiments, the subject is a mammal including a non-primate (e.g., a camel, donkey, zebra, bovine, horse, horse, cat, dog, rat, and mouse) and a primate (e.g., a monkey, chimpanzee, and a human). In some embodiments, the subject is a non-human mammal. In certain embodiments, the subject is a pet (e.g., dog or cat) or farm animal (e.g., a horse, pig or cow). In specific embodiments, the subject is a human. In certain embodiments, the mammal (e.g., human) is 4 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old. In specific embodiments, the subject is an animal that is not avian.

As used herein, the terms “therapies” and “therapy” can refer to any protocol(s), method(s), agent(s) or a combination thereof that can be used in the treatment cancer. In certain embodiments, the term “therapy” refers to an oncolytic virus described herein (e.g., an APMV). In other embodiments, the term “therapy” refers to an agent that is not an oncolytic virus described herein (e.g., an APMV).

### 4. BRIEF DESCRIPTION OF THE FIGURES

FIG. 1. Rescue of rNDV from a cloned cDNA. BSR-T7 cells growing in a 6 well plate are infected with virus MVA-T7, to express the T7 RNA polymerase. After 1 h., cells are transfected with plasmid pNDV-LaSota-L289A, and helper plasmids pTM1.NP, pTM1.P and pTM1.L. Next day cells and supernatants are inoculated into 10 day-old embryonated chicken eggs to amplify the rescued virus. After 3 days the allantoic fluid is harvested and analyzed for the presence of virus by hemagglutination (HA) assay. HA positive samples are further characterized to confirm the presence and expression of the inserted gene.

FIG. 2. Schematic of the protocol used for the construction of the rescue plasmid pNDV-LaSota-L289A-mVEGF-C. (Not to scale). The open reading frame encoding the murine VEGF-C (mVEGF-C) protein is amplified by PCR using primers that incorporate additional sequences: Forward primer: Sac II restriction site+NDV regulatory sequences (gene end+intergene+gene start)+Kozac sequences for optimal translation. Reverse primer: additional nucleotides (rule of 6)+Sac II restriction site. Next, the amplified PCR product is cloned into the Sac II site of plasmid pNDV-LaSota-L289A to generate the rescue plasmid pNDV-LaSota-L289A-mVEGF-C. Primer sequences are provided in Table 1.

FIG. 3. Schematic of the protocol used for the cloning of a full-length cDNA of the APMV4 genome with engineered unique restriction sites. Using purified viral RNA as template, each viral gene was amplified by RT-PCR with primers that introduced unique restriction sites as indicated. Next, PCR products 1.1, 1.2 and 1.3 (viral genes NP, P and M, respectively) were cloned in the multicloning site of plasmid pUC-18 to generate plasmid pUC-APMV4-1. PCR products 2.1 and 2.2 (genes F and HN, respectively) were cloned into pUC-APMV4-2 and PCR products 3.1 and 3.2 (gene L) were cloned into pUC-APMV4-3. Next, plasmids 1 and 2 were combined to create pUC-APMV4-1+2 and finally plasmids 1+2 and 3 were combined to generate pUC-APMV4-1+2+3 that contains a full-length copy of the APMV4 genome with engineered unique restriction sites between each viral gene. Primer sequences are provided in Table 1.

FIG. 4. Schematic of the protocol used for the cloning of the helper plasmids expressing APMV4 proteins NP, P and L. (Not to scale) Using as template plasmids pUC-APMV4-1 and pUC-APMV4-3, the open reading frames coding the viral proteins NP, P and L were amplified by PCR. Next, the amplified PCR products were cloned into the pTM1 vector using the Nco I and Pst I restriction sites. Primer sequences are provided in Table 1.

FIG. 5. Schematic of the protocol used for the rescue of rAPMV4 from a cloned cDNA. BSR-T7 cells growing in a 6 well plate are infected with virus MVA-T7, to express the T7 RNA polymerase. After 1 h., cells are transfected with plasmid pRz-APMV4, and helper plasmids pTM1-APMV4.NP, pTM1-APMV4.P and pTM1-APMV4.L. Next day cells and supernatants are inoculated into 10 day-old embryonated chicken eggs to amplify the rescued virus. After 3 days the allantoic fluid is harvested and analyzed for the presence of virus by hemagglutination (HA) assay. HA positive samples are further characterized to confirm the presence and expression of the inserted gene.

FIG. 6. Schematic of the protocol used for the construction of the rescue plasmid pRz-APMV4-mVEGF-C. (Not to scale). The rescue plasmid containing a codon optimized mVEGF-C gene is constructed in 2 steps. First, a synthetic DNA encoding a codon optimized mVEGF-C protein is amplified by PCR and cloned at the unique Sal I site of plasmid pUC-APMV4-1. Next, a Nhe I-Sbf I is replaced in the plasmid pRz-APMV4 to generate the rescue plasmid pRz-APMV4-mVEGF-C. Primer sequences are provided in Table 1.

FIGS. 7A-7E. Oncolytic activity of APMVs (namely, NDV LS289A or APMV-4) in B16-F10 and B16-VEGF-C+ syngeneic murine melanoma tumor model. FIG. 7A shows a schematic of the experimental set up for Study 1. FIG. 7B shows an analysis of tumor growth rate. Points represent average of tumor volume per experimental group at the indicated time point. Error bars correspond to standard deviation of each group. FIG. 7C shows individual tumor growth curves. Each point represents tumor volume per mice at the indicated time point. FIG. 7D shows overall survival. FIG. 7E shows a comparative analysis between experimental groups, of treated B16-F10 or B16-VEGF-C+ tumor-bearing mice.

FIGS. 8A-8C. Re-challenge. FIG. 8A: Right panel: schematic of the re-challenge experimental set up for the Study 1. Left panel: analysis of tumor growth rate. Points represent average of tumor volume per experimental group at the indicated time point. Error bars correspond to standard deviation of each group. FIG. 8B: individual tumor growth curves. Each point represents tumor volume per mice at the indicated time point. FIG. 8C: post-re-challenge overall survival analysis of Study 1.

FIGS. 9A-9D. Oncolytic activity of APMVs (namely, NDV LS289A or APMV-4) in B16-F10 and B16-VEGF-C+ syngeneic murine melanoma tumor models. FIG. 9A shows a schematic of the experimental set up for Study 2. FIG. 9B shows an analysis of tumor growth rate. Points represent average of tumor volume per experimental group at the indicated time point. Error bars correspond to standard deviation of each group. FIG. 9C shows individual tumor growth curves. Each point represents tumor volume per mice at the indicated time point. FIG. 9D shows overall survival analysis pre-re-challenge.

FIGS. 10A-10D. Re-challenge. FIG. 10A shows a schematic of the re-challenge experimental set up for Study 2. FIG. 10B shows analysis of tumor growth rate. Points represent average of tumor volume per experimental group at the indicated time point. Error bars correspond to standard deviation of each group. FIG. 10C shows survival post-re-challenge. FIG. 10D shows survival analysis summary for Study 2.

FIGS. 11A-11C: VEGF-C potentiates anti-tumor immune response stimulated by the viral dsRNA mimic poly(I:C). FIG. 11A shows a schematic of the experimental set up. FIG. 11B shows analysis of tumor growth rate. Points represent average of tumor volume per experimental group, error bars indicate standard deviation. FIG. 11C shows individual tumor growth curves.

FIG. 12. Schematic representation of the viral constructs overexpressing full-length or mature VEGF-C. PT7, T7 promoter NP, nucleoprotein; P, phosphoprotein; M, matrix protein, F, fusion protein; HN, hemagglutinin-neuraminidase; L, large polymerase protein. HDR, hepatitis delta ribozyme. TT7, T7 terminator sequence.

FIGS. 13A-13C. Characterization of VEGF-C expression in cells transduced with NDV/VEGF-C wt constructs. FIG. 13A: Immunofluorescent staining of Vero cells infected with NDV/VEGF-C FL-WT using an anti-VEGF-C antibody. FIG. 13B: ELISA of conditioned media from 293T cells transduced with the NDV/VEGF-C constructs as indicated (full length and mature forms, WT and mutants thereof). Conditioned media was collected after 24 hours. FIG. 13C: Western blot of supernatants from 293T cells transduced with NDV/VEGF-C constructs as indicated. Scale bars: 50 μm.

FIGS. 14A-14F. Effects of NDV/VEGF-C FL-WT and dNdC-WT on B16F10 tumors. FIG. 14A: Schematic representation of the NDV viral treatment schedule. FIG. 14B: Effects of treatment of tumors with NDV/VEGF-C FL-WT construct in comparison to NDV on survival of mice bearing B16F10 tumors. FIG. 14C: Tumor growth curves with different treatments as indicated. Each line represents tumor from one mouse. FIG. 14D: Tumor growth curves showing average values for each treatment type. FIG. 14E: Survival data for each of the treatments as indicated. FIG. 14F: Immunohistochemical staining (IHC) of tumors treated with NDV or NDV/VEGF-C FL-WT for VEGF-C, LYVE-1 and CD8, as indicated.

FIGS. 15A-15F. Effects of NDV treatment and VEGF-C on tumor growth and long-term survival of mice with B16F10 tumors. FIG. 15A: Experimental design showing tumor treatment schedule. FIG. 15B Tumor growth of B16F10 cells transfected with control vector not expressing VEGF-C, and treated with NDV. FIG. 15C: Tumor growth of B16F10 cells constitutively expressing VEGF-C and treated with NDV. FIG. 15D: Mouse post-treatment of B16F10/VEGF-C tumor with NDV. Note white patches of hair at the sites where tumors regressed. FIG. 15E: Survival of mice following treatments as indicated. FIG. 15F: Tumor growth at initial injection and following re-challenge of survivor mice, as indicated.

FIGS. 16A-16F. Immunophenotyping of B16F10 tumors by Aurora spectral flow cytometry. FIG. 16A: Distribution of T-cell and NK cell phenotypes in B16F10/VEGF-C tumors treated with NDV, compared to B16F10 mock control. FIG. 16B: Distribution of T-cell and NK cell phenotypes in B16F10/VEGF-C tumors treated with NDV, compared to B16F10 treated with NDV. FIG. 16C: Distribution of immune cell phenotypes in B16F10 and B16F10/VEGF-C tumors treated as indicated. Flow cytometry data for T-cell and NK cell activation markers. FIG. 16D: Fraction of activated vs. all CD45+ immune cells in tumors treated as indicated. FIG. 16E: Fraction of activated cells across different treatment groups. FIG. 16F: Fraction of main activated immune cell subtypes in VEGF-C/NDV group. Question marks indicate that the exact immune cell subset could not be determined based on the marker combination.

FIG. 17A-17M. Effects of NDV treatment and VEGF-C on the distribution of immune cells in tumors. Effects of NDV treatment and VEGF-C on the distribution of immune cells in tumors. FIGS. 17A-17C: Immunofluorescent staining of B16F10 PBS-treated tumors for CD8 (FIG. 17A), CD4 (FIG. 17B), and CD11c (FIG. 17C). FIGS. 17D-17F: Immunofluorescent staining of B16F10/VEGF-C NDV-treated tumors for CD8 (FIG. 17D), CD4 (FIG. 17E), and CD11c (FIG. 17F). FIGS. 17 G and 17H: Immunofluorescent staining for lymphatics (LYVE-1) in tumors as indicated and (FIG. 17I) for CD8+ T-cells in the same section shown in (FIG. 17H). Note high CD8+ T-cell densities in tumor areas with high lymphatic vessel densities. (FIG. 17J) Quantification of CD8+ T-cells in tumors based on immunostaining. (K) Conventional flow cytometry analysis of immune cells in tumors. CD8+CD25+ effector memory T-cells are shown. FIGS. 17L and 17M: Quantification of lymphatic (FIG. 17L) and blood (FIG. 17M) vessel densities in tumors as indicated. EV/PBS, B16F10 cells transfected with empty vector control and tumors treated with PBS. VEGF-C/NDV, B16F10 cells transfected with VEGF-C and tumors treated with NDV.

FIGS. 18A-18B. Immunophenotyping of lymph nodes draining B16F10 tumors by Aurora spectral flow cytometry. FIG. 18A: Distribution of immune cell phenotypes in sentinel lymph nodes of B16F10 and B16F10/VEGF-C tumors treated as indicated. FIG. 18B: Distribution of immune cell phenotypes in contralateral lymph nodes of B16F10 and B16F10/VEGF-C tumors treated as indicated. Question marks indicate that the exact immune cell subset could not be determined based on the marker combination.

### 5. DETAILED DESCRIPTION

### 5.1 Oncolytic Viruses

In one aspect, provided herein are viruses that may be used in a method for treating cancer described herein. In some embodiments, the virus can be any virus known in the art, including, e.g., an adeno-associated virus (“AAV”; e.g., AAV1-AAV9). In other embodiments, the virus is not an adeno-associate virus (e.g., is not AAV1-AAV9). In a specific aspect, provided herein are oncolytic viruses that may be used in a method for treating cancer described herein. In a specific embodiment, an oncolytic virus is a virus that when injected into a tumor results in tumor regression. In another specific embodiment, an oncolytic virus is a virus that selectively replicates in and kills cancer cells, and spreads within the tumor. In another specific embodiment, an oncolytic virus is a virus that selectively replicates in and kills cancer cells, and spreads within the tumor without causing any significant damage to normal tissue. In some embodiments, an in vitro or ex vivo assay known to one skilled in the art is used to determine the selectively of a virus to replicate in cancer cells versus non-cancerous cells (e.g., healthy cells). In one embodiment, a virus selectively replicates in cancer cells if a statistically significant increase in the number of virus particles is detected in cancer cells in an in vitro assay or ex vivo assay relative to the number of virus particles detected in non-cancerous cells (e.g., healthy cells) in the same assay after incubation with the virus. In another embodiment, a virus selectively kills cancer cells if a statistically significant amount of the cancer cells are killed in an in vitro or ex vivo assay relative to the amount of non-cancerous cells (e.g., healthy cells) killed in the same assay. In one embodiment, an oncolytic virus naturally preferentially replicates in cancer cells and is non-pathogenic in humans. An oncolytic virus may be non-pathogenic in humans due to elevated sensitivity to innate antiviral signal or dependence on oncogenic signaling pathways. In some embodiments, an oncolytic virus is a parovirus (e.g., an autonomous parvovirus), a myxoma virus, an avian paramyxovirus (e.g., Newcastle disease virus), a reovirus, or Seneca valley virus. In one embodiment, an oncolytic virus is wild-type parvovirus H1 (ParvOryx). In another embodiment, an oncolytic virus is Vesicular stomatitis virus. In another embodiment, an oncolytic virus is an avian paramyxovirus. See Section 5.1.1, infra, regarding avian paramyxoviruses.

In certain embodiments, an oncolytic virus is a virus that is genetically engineered with mutations (e.g., deletions and/or substitutions) in genes required for replication in normal, but not cancer cells. In some embodiments, an oncolytic virus is a genetically engineered influenza virus, measles virus, poliovirus, vaccinia virus, poxvirus, picornavirus, alphavirus, retrovirus, rhabdovirus, reovirus, adenovirus, herpes simplex virus, or vesicular stomatitis virus. In a specific embodiment, such viruses are attenuated. In one embodiment, an oncolytic virus is an E1A/E1B-deleted adenovirus (ONYX015) (see, e.g., Cohen and Rudin, 2001, Curr. Opin. Investig. Drugs 2(12): 1770-1775, which is incorporated by reference in its entirety, for information regarding ONYX015. In another embodiment, an oncolytic virus is the adenovirus is H101, a conditionally replicative adenovirus, was generated by both E1B and E3 gene deletion (see, e.g., Kasuya et al., 2007, Curr Cancer Drug Targets. 7:123-125, which is incorporated by reference in its entirety, for information regarding H101). In another embodiment, an oncolytic virus is adenovirus known as Delta-24-RGD (DNX-2401). In another embodiment, an oncolytic virus is an attenuated influenza virus, (e.g., an influenza virus comprising a truncated NS1 protein such as described in U.S. Pat. Nos. 10,098,945; 8,057,803; 8,124,101; 8,137,676; 6,866,853; 6,669,943; 6,468,544; 8,137,676; and 9,387,240, each of which is incorporated herein by reference in its entirety). In another embodiment, an oncolytic virus is HSV1716 (Seprehvir®). In another embodiment, an oncolytic virus is G207. In another embodiment, an oncolytic virus is Pelareorep (Reolysin®). In certain embodiments, an oncolytic virus for use in a method for treating cancer described herein may be engineered to express a heterologous protein. For example, an oncolytic virus may be engineered to express a checkpoint inhibitor (e.g., an antibody that specifically binds to PD-1 and blocks binding of PD-1 to PDL1, PDL2 or both, such as pembrolizumab or nivolumab; an antibody that specifically binds to PDL1 and blocks binding of PDL1 to PD1, CD80 or both, such as atezolizumab, durvalumab, or cemiplimab; an antibody that specifically binds to CLTA-4 and block the interaction of CTLA-4 with its ligands B7.1 and B7.2, such as ipilimumab or tremelimumab; and an antibody that specifically binds to TIM-3); a cytokine (e.g., IL-2, IL-12, IL-15, IFN alpha/beta, 41-BB, CD40L, Flt3L, CCL3, CCL5, GM-CSF, etc.); an agonist of a co-stimulatory receptor (e.g., an agonist of ICOS, ICOS-L, OX40, OX40L, etc.); or a cancer antigen (e.g., a tumor associated antigen). See, e.g., Section 5.7.2 for examples of checkpoint inhibitors and agonists of co-stimulatory receptors. In some embodiments, an oncolytic virus for use in a method for treating cancer described herein is pexastimogene devacirepvec (Pexa-Vec, formerly named JX-594), ONCOS (adeno Δ24-RGD-GM-CSF insertion), herpes virus OrienX010, ICOVIR-5, Talimogene Laherparepvec (T-VEC, Imlygic®), VV JX-594, Ad Ad5/3-D24-GMCSF, or CG0070. In other embodiments, an oncolytic virus is not engineered to express a checkpoint inhibitor, a cytokine, an agonist of a co-stimulatory receptor, or a cancer antigen.

In certain embodiments, an oncolytic virus for use in a method for treating cancer described herein is not engineered to express a heterologous protein. In some embodiments, an oncolytic virus for use in a method for treating cancer is not engineered to express a cancer antigen (e.g., a tumor associated antigen). In certain embodiments, an oncolytic virus for use in a method for treating cancer is not engineered to express a heterologous viral antigen. In some embodiments, an oncolytic virus for use in a method for treating cancer is not engineered to express a bacterial antigen, a fungal antigen, a protozoal antigen, or a helminth antigen.

### 5.1.1 Avian Paramyxoviruses

Any APMV-1 (otherwise known as Newcastle disease virus or NDV), APMV-2, APMV-3, APMV-4, APMV-5, APMV-6, APMV-7, APMV-8, or APMV-9 strain, including, but not limited to, naturally-occurring strains, variants or mutants, mutagenized viruses, genetically engineered viruses, or a combination thereof may be used in the methods for treating cancer described herein. See Table 2 for exemplary APMV sequences. One skilled in the art would understand that viruses may undergo mutation when cultured, passaged or propagated. The APMV-1, APMV-2, APMV-3, APMV-4, APMV-5, APMV-6, APMV-7, APMV-8, or APMV-9 strain may contain these naturally occurring mutations, in addition to mutations introduced for cloning purposes. The APMV-2, APMV-3, APMV-4, APMV-5, APMV-6, APMV-7, APMV-8, or APMV-9 strain may be a homogenous or heterogeneous population with none, or one or more of these mutations. In certain embodiments, the APMV-1, APMV-2, APMV-3, APMV-4, APMV-5, APMV-6, APMV-7, APMV-8, or APMV-9 strain that is used in a method of treating cancer described herein is a lytic strain. In other embodiments, the APMV-1, APMV-2, APMV-3, APMV-4, APMV-5, APMV-6, APMV-7, APMV-8, or APMV-9 strain that is used in a method of treating cancer described herein is a non-lytic strain. In a specific embodiment, the APMV-1, APMV-2, APMV-3, APMV-4, APMV-5, APMV-6, APMV-7, APMV-8, or APMV-9 strain that is used in a method of treating cancer described herein is naturally occurring. In a specific embodiment, the APMV-1, APMV-2, APMV-3, APMV-4, APMV-5, APMV-6, APMV-7, APMV-8, or APMV-9 strain that is used in a method of treating cancer described herein is avirulent in an avian(s) by a method(s) described herein or known to one of skill in the art. In a specific embodiment, the APMV-1, APMV-2, APMV-3, APMV-4, APMV-5, APMV-6, APMV-7, APMV-8, or APMV-9 strain that is used in a method of treating cancer described herein is recombinantly produced. In certain embodiments, the APMV-1, APMV-2, APMV-3, APMV-4, APMV-5, APMV-6, APMV-7, APMV-8, or APMV-9 strain that is used in a method of treating cancer described herein is genetically engineered to be attenuated in a manner that attenuates the pathogenicity of the virus in birds.

In another specific embodiment, the APMV-1, APMV-2, APMV-3, APMV-4, APMV-6, APMV-7, APMV-8, or APMV-9 strain that is used in a method of treating cancer described herein has an intracerebral pathogenicity index in day-old chicks of the Gallus gallus species of less than 0.7. In certain specific embodiments, the APMV-1, APMV-2, APMV-3, APMV-4, APMV-5, APMV-6, APMV-7, APMV-8, or APMV-9 strain that is used in a method of treating cancer described herein is not pathogenic as assessed by intracranial injection of 1-day-old chicks with the virus, and disease development and death as scored for 8 days. In some embodiments, the APMV-1, APMV-2, APMV-3, APMV-4, APMV-5, APMV-6, APMV-7, APMV-8, or APMV-9 strain that is used in a method of treating cancer described herein has an intracranial pathogenicity index of less than 0.7, less than 0.6, less than 0.5, less than 0.4, less than 0.3, less than 0.2 or less than 0.1. In some embodiments, the APMV-1, APMV-2, APMV-3, APMV-4, APMV-5, APMV-6, APMV-7, APMV-8, or APMV-9 strain that is used in a method of treating cancer described herein has an intracranial pathogenicity index between 0.7 to 0.1, 0.6 to 0.1, 0.5 to 0.1 or 0.4 to 0.1. In certain embodiments, the APMV-1, APMV-2, APMV-3, APMV-4, APMV-5, APMV-6, APMV-7, APMV-8, or APMV-9 strain that is used in a method of treating cancer described herein has an intracranial pathogenicity index of zero. See, e.g., one or more of the following references for a description of an assay that may be used to assess the pathogenicity of an APMV in birds: Hines, N. L. and C. L. Miller, Avian paramyxovirus serotype-1: a review of disease distribution, clinical symptoms, and laboratory diagnostics. Vet Med Int, 2012. 2012: p. 708216; Kim S-H, Xiao S, Shive H, Collins P L, Samal S K., 2012: Replication, Neurotropism, and Pathogenicity of Avian Paramyxovirus Serotypes 1-9 in Chickens and Ducks. PLoS ONE. 7(4): e34927; Subbiah, M., Xiao, S., Khattar, S. K., Dias, F. M., Collins, P. L., & Samal, S. K., 2010: Pathogenesis of two strains of Avian Paramyxovirus serotype 2, Yucaipa and Bangor, in chickens and turkeys. Avian Diseases, 54(3), 1050-1057; Kumar S, Militino Dias F, Nayak B, Collins P L, Samal S. K., 2010: Experimental avian paramyxovirus serotype-3 infection in chickens and turkeys. Veterinary Research.; 41(5):72; Ryota Tsunekuni, Hirokazu Hikono, Takehiko Saito., 2014: Evaluation of avian paramyxovirus serotypes 2 to 10 as vaccine vectors in chickens previously immunized against Newcastle disease virus. Veterinary Immunology and Immunopathology; 160(3-4):184-191; and www.oie.int/fileadmin/Home/fr/Health_standards/tahm/2.03.14 NEWCASTLE DIS.pdf, each of which is incorporated herein by reference in its entirety. In a specific embodiment, the APMV-1, APMV-2, APMV-3, APMV-4, APMV-5, APMV-6, APMV-7, APMV-8, or APMV-9 strain is a recombinant APMV-1, APMV-2, APMV-3, APMV-4, APMV-5, APMV-6, APMV-7, APMV-8, or APMV-9 strain, respectively.

In another specific embodiment, the APMV-1, APMV-2, APMV-3, APMV-4, APMV-5, APMV-6, APMV-7, APMV-8, or APMV-9 strain that is used in a method of treating cancer described herein is naturally occurring and has an intracerebral pathogenicity index in day-old chicks of the Gallus gallus species of less than 0.7. In a specific embodiment, the APMV-1, APMV-2, APMV-3, APMV-4, APMV-5, APMV-6, APMV-7, APMV-8, or APMV-9 strain that is used in a method of treating cancer described herein is a recombinant APMV-1, APMV-2, APMV-3, APMV-4, APMV-5, APMV-6, APMV-7, APMV-8, or APMV-9 strain, respectively, and has an intracerebral pathogenicity index in day-old chicks of the Gallus gallus species of less than 0.7.

In a specific embodiment, an APMV-1, APMV-2, APMV-3, APMV-4, APMV-5, APMV-6, APMV-7, APMV-8, or APMV-9 that is used in a method of treating cancer described herein decreases tumor growth and increases survival in a B16-F10-VEGF-C+ syngeneic murine melanoma model as compared to tumor growth and survival in B16-F10-VEGF-C+ syngeneic murine melanoma model administered phosphate buffered saline (PBS) as assessed by a method known in the art or described herein (e.g., in Section 6, infra).

In a specific embodiment, an APMV strain is used in a method for treating cancer described herein is an AMPV-1 or APMV-4 described in Section 6, infra.

In one embodiment, an APMV-2 strain is used in a method for treating cancer described herein, wherein the APMV-2 strain is APMV-2 Chicken/California/Yucaipa/1956. See, e.g., GenBank No. EU338414.1 for the complete genomic cDNA sequence of APMV-2 Chicken/California/Yucaipa/1956. In another embodiment, an APMV-3 strain is used in a method for treating cancer described herein, wherein the APMV-3 strain is APMV-3 turkey/Wisconsin/68. See, e.g., GenBank No. EU782025.1 for the complete genomic cDNA sequence of APMV-3 turkey/Wisconsin/68. In another embodiment, an APMV-6 strain is used in a method for treating cancer described herein, wherein the APMV-6 strain is APMV-6/duck/Hong Kong/18/199/77. See, e.g., GenBank No. EU622637.2 for the complete genomic cDNA sequence of APMV-6/duck/Hong Kong/18/199/77. In another embodiment, an APMV-7 strain is used in a method for treating cancer described herein, wherein the APMV-7 strain is APMV-7/dove/Tennessee/4/75. See, e.g., GenBank No. FJ231524.1 for the complete genomic cDNA of APMV-7/dove/Tennessee/4/75. In another embodiment, an APMV-8 strain is used in a method for treating cancer described herein, wherein the APMV-8 strain is APMV-8/Goose/Delaware/1053/76. See, e.g., GenBank No. FJ619036.1 for the complete genomic cDNA sequence of APMV-8/Goose/Delaware/1053/76. In another embodiment, an APMV-9 is used in a method for treating cancer described herein, wherein the APMV-9 strain is APMV-9 duck/New York/22/1978. See, e.g., GenBank No. NC 025390.1 for the complete genomic cDNA sequence of APMV-9 duck/New York/22/1978.

In a specific embodiment, an APMV-1 is used in a method for treating cancer described herein. In a specific embodiment, the APMV-1 used in a method for treating cancer described herein is a naturally-occurring strain. In certain embodiments, the APMV-1 is a lytic strain. In other embodiments, the APMV-APMV-1 used in a method for treating cancer described herein is a non-lytic strain. In certain embodiments, the APMV-1 used in a method for treating cancer described herein is lentogenic strain. In some embodiments, the APMV-1 used in a method for treating cancer described herein is a mesogenic strain. In other embodiments, the APMV-1 used in a method for treating cancer described herein is a velogenic strain. See, e.g., Newcastle Disease, Avian Paramyoxvirus-1 Infection, Goose Paramyoxvirus Infection, Ranikhet disease, the Center for Food Security & Public Health, Iowa State University, Institute for International Cooperation in Animal Biologies, College of Veterinary Medicine, Iowa State University, pp. 1-9 (January 2016) for a discussion regarding lentogenic, mesogenic and velogenic APMV-1 (otherwise referred to as NDV) strains, which is incorporated herein by reference in its entirety. Specific examples of APMV-1 strains include, but are not limited to, the 73-T strain, NDV HUJ strain, Ulster strain (see, e.g., GenBank No. U25837), MTH-68 strain, Italien strain (see, e.g., GenBank No. EU293914), Hickman strain (see, e.g., Genbank No. AF309418), PV701 strain, Hitchner B1 strain (see, e.g., GenBank No. AF309418 or NC 002617), La Sota strain (see, e.g., GenBank Nos. AY845400 and JF950510.1 and GI No. 56799463), YG97 strain (see, e.g., GenBank Nos. AY351959 or AY390310), MET95 strain (see, e.g., GenBank No. AY143159), Roakin strain (see, e.g., GenBank No. AF124443), and F48E9 strain (see, e.g., GenBank Nos. AF163440 and U25837). In a specific embodiment, the APMV-1 used in a method for treating cancer described herein that is the Hitchner B 1 strain. In another specific embodiment, the APMV-1 used in a method for treating cancer described herein is a B 1 strain as identified by GenBank No. AF309418 or NC_002617. In another specific embodiment, the APMV-1 used in a method for treating cancer described herein is the NDV identified by ATCC No. VR2239. In another specific embodiment, the APMV-1 used in a method for treating cancer described herein is an NDV described in U.S. Pat. No. 10,035,984, which is incorporated herein by reference in its entirety.

In a specific embodiments, an APMV-1 that is used in a method of treating cancer described herein is genetically modified. In one embodiment, a genome of an APMV-1 used in a method of treating cancer described herein is engineered to express a mutated F protein with a mutated cleavage site. In a specific embodiment, the APMV-1 used in a method of treating cancer described herein is engineered to express a mutated F protein in which the cleavage site of the F protein is mutated to produce a polybasic amino acid sequence, which allows the protein to be cleaved by intracellular proteases, which makes the virus more effective in entering cells and forming syncytia. In another specific embodiment, the APMV-1 used in a method of treating cancer described herein is engineered to express a mutated F protein in which the cleavage site of the F protein is replaced with a mutated cleavage site containing one or two extra arginine residues, allowing the mutant cleavage site to be activated by ubiquitously expressed proteases of the furin family. Specific examples of NDVs that express such a mutated F protein include, but are not limited to, rNDV/F2aa and rNDV/F3aa. For a description of mutations introduced into a NDV F protein to produce a mutated F protein with a mutated cleavage site, see, e.g., Park et al. (2006) Engineered viral vaccine constructs with dual specificity: avian influenza and Newcastle disease. PNAS USA 103: 8203-2808, which is incorporated herein by reference in its entirety.

In a specific embodiment, an APMV-1 strain is used in a method for treating cancer described herein. In another embodiment, an APMV-1 strain that is naturally occurring is used in a method of treating cancer described herein. In a specific embodiment, an APMV-1 strain that is naturally occurring and has an intracerebral pathogenicity index in day-old chicks of the Gallus gallus species of less than 0.7 is used in a method of treating cancer described herein. In a specific embodiment, an APMV-1 that is used in a method of treating cancer described herein is an APMV-1 with a genome that has 80%, 85%, 90%, 95% or higher percent identity to the genome of a LaSota strain (e.g., SEQ ID NO: 83 or 84).

In a specific embodiment, an APMV-1 used in a method of treating cancer described herein is engineered to express a mutated F protein with the amino acid mutation L289A (i.e., an L to A mutation at the amino acid position corresponding to L289 of the LaSota F protein). For a description of the L289A mutation, see, e.g., Sergei et al. (2000) A Single Amino Acid Change in the Newcastle Disease Virus Fusion Protein Alters the Requirement for UN Protein in Fusion. Journal of Virology 74(11): 5101-5107, which is incorporated herein by reference in its entirety. In specific embodiments, the L289A mutated F protein possesses one, two or three arginine residues in the cleavage site. In a specific embodiment, the APMV-1 used in a method of treating cancer described herein is the LaSota strain, which has been engineered to express a mutated F protein with the amino acid mutation L289A (i.e., an L to A mutation at the amino acid position corresponding to L289 of the LaSota F protein). In a specific embodiment, the genetically modified NDV LaSota strain comprises a packaged genome, wherein the packaged genome comprises the negative sense RNA transcribed from the cDNA sequence set forth in SEQ ID NO: 83 or 84.

In some embodiments, an APMV-1 used in a method of treating cancer described herein is the NDV disclosed in Kim et al., 2017, PLOS ONE 12(3): e0173965 and Kim et al., 2016, J. of General Virology 97: 1297-1303, each of which is incorporated herein by reference in its entirety.

In certain embodiments, an APMV-1 used in a method of treating cancer described herein comprises a mutated F protein with an F protein cleavage site of NDV LaSota strain or glycoprotein B of cytomegalovirus (CMV). In a specific embodiment, an APMV-1 used in a method of treating cancer described herein comprises a mutated F protein with an F protein cleavage having one of the following sequence modifications: S116: ¹¹¹H-N-R-T-K-S/F¹¹⁷ (SEQ ID NO: 91); S116K: ¹¹¹H-N-K-T-K-S/F¹¹⁷ (SEQ ID NO: 92); S116M: ¹¹¹H-N-R-M-K-S/F¹¹⁷ (SEQ ID NO: 93); S116KM: ¹¹¹H-N-K-M-K-S/F-I¹¹⁸ (SEQ ID NO: 94); or R116: ¹¹¹H-N-R-T-K-R/F-I¹¹⁸ (SEQ ID NO: 95), such as described in International Patent Application No. WO 2015/032755. See, e.g., International Patent Application Publication No. WO 2015/032755 for a description of the types of mutated F protein cleavage sites that may be engineered into an NDV F protein, which is incorporated herein by reference in its entirety. In some embodiments, the mutated F protein is in addition to the backbone NDV F protein. In specific embodiments, the mutated F protein replaces the backbone NDV F protein.

In a specific embodiment, an APMV-4 strain is used in a method for treating cancer described herein. In another embodiment, an APMV-4 strain that is naturally occurring is used in a method of treating cancer described herein. In a preferred embodiment, an APMV-4 strain that is naturally occurring and has an intracerebral pathogenicity index in day-old chicks of the Gallus gallus species of less than 0.7 is used in a method of treating cancer described herein. In a preferred embodiment, the APMV-4 that is used in a method of treating cancer described herein is APMV-4/Duck/Hong Kong/D3/1975 strain. See, e.g., GenBank No. FJ177514.1 or SEQ ID NO: 78 for the complete genomic cDNA sequence of APMV-4/duck/Hong Kong/D3/75. In a specific embodiment, an APMV-4 that is used in a method of treating cancer described herein is APMV-4/Duck/China/G302/2012 strain, APMV4/mallard/Belgium/15129/07 strain, APMV4/Uriah_aalge/Russia/Tyuleniy_Island/115/2015 strain, APMV-4/Egyptian goose/South Africa/N1468/2010 strain, or APMV4/duck/Delaware/549227/2010 strain. In a specific embodiment, an APMV-4 that is used in a method of treating cancer described herein is an APMV-4 with a genome that has 80%, 85%, 90%, 95% or higher percent identity to the genome of APMV-4/Duck/Hong Kong/D3/1975 strain. In one embodiment, an APMV-4 that is used in a method of treating cancer described herein is APMV-4/Duck/China/G302/2012 strain. See, e.g., GenBank No. KC439346.1 or SEQ ID NO: 81 for the complete genomic cDNA sequence of APMV-4/Duck/China/G302/2012 strain. In a specific embodiment, an APMV-4 that is used in a method of treating cancer described herein is an APMV-4 with a genome that has 80%, 85%, 90%, 95% or higher percent identity to the genome of APMV-4/Duck/China/G302/2012 strain. In another embodiment, an APMV-4 that is used in a method of treating cancer described herein is APMV-4/Uriah_aalge/Russia/Tyuleniy_Island/115/2015 strain. See, e.g., GenBank No. KU601399.1 or SEQ ID NO: 79 for the complete genomic cDNA sequence of APMV-4/Uriah_aalge/Russia/Tyuleniy_Island/115/2015 strain. In a specific embodiment, an APMV-4 that is used in a method of treating cancer described herein is an APMV-4 with a genome that has 80%, 85%, 90%, 95% or higher percent identity to the genome of APMV-4/Uriah_aalge/Russia/Tyuleniy_Island/115/2015 strain. In another embodiment, the APMV-4 that is used in a method of treating cancer described herein is APMV4/duck/Delaware/549227/2010 strain. See, e.g., GenBank No. JX987283.1 or SEQ ID NO: 82 for the complete genomic cDNA sequence of APMV4/duck/Delaware/549227/2010 strain. In a specific embodiment, an APMV-4 that is used in a method of treating cancer described herein is an APMV-4 with a genome that has 80%, 85%, 90%, 95% or higher percent identity to the genome of APMV4/duck/Delaware/549227/2010 strain. In another embodiment, an APMV-4 that is used in a method of treating cancer described herein is APMV4/mallard/Belgium/15129/07 strain. See, e.g., GenBank No. JN571485 or SEQ ID NO: 77 for the complete genomic cDNA sequence of APMV4/mallard/Belgium/15129/07 strain. In a specific embodiment, an APMV-4 that is used in a method of treating cancer described herein is an APMV-4 with a genome that has 80%, 85%, 90%, 95% or higher percent identity to the genome of APMV4/mallard/Belgium/15129/07 strain. In another embodiment, the APMV-4 that is used in a method of treating cancer described herein is APMV-4/Egyptian goose/South Africa/N1468/2010 strain. See, e.g., GenBank No. JX133079.1 or SEQ ID NO: 80 for the complete genomic cDNA sequence of APMV-4/Egyptian goose/South Africa/N1468/2010 strain. In a specific embodiment, an APMV-4 that is used in a method of treating cancer described herein is an APMV-4 with a genome that has 80%, 85%, 90%, 95% or higher percent identity to the genome of APMV-4/Egyptian goose/South Africa/N1468/2010 strain.

In a specific embodiment, an APMV-8 strain is used in a method for treating cancer described herein. In another embodiment, an APMV-8 strain that is naturally occurring is used in a method of treating cancer described herein. In a specific embodiment, an APMV-8 strain that is naturally occurring and has an intracerebral pathogenicity index in day-old chicks of the Gallus gallus species of less than 0.7 is used in a method of treating cancer described herein. In a specific embodiment, an APMV-8 that is used in a method of treating cancer described herein is APMV-8/Goose/Delaware/1053/76. See, e.g., GenBank No. FJ619036.1 for the complete genomic cDNA sequence of APMV-8/Goose/Delaware/1053/76. In a specific embodiment, an APMV-8 that is used in a method of treating cancer described herein is an APMV-8 with a genome that has 80%, 85%, 90%, 95% or higher percent identity to the genome of APMV-8/Goose/Delaware/1053/76.

In a specific embodiment, an APMV-7 strain is used in a method for treating cancer described herein. In another embodiment, an APMV-7 strain that is naturally occurring is used in a method of treating cancer described herein. In a preferred embodiment, an APMV-7 strain that is naturally occurring and has an intracerebral pathogenicity index in day-old chicks of the Gallus gallus species of less than 0.7 is used in a method of treating cancer described herein. In a specific embodiment, an APMV-7 that is used in a method of treating cancer described herein is APMV-7/dove/Tennessee/4/75. See, e.g., GenBank No. FJ231524.1 for the complete genomic cDNA of APMV-7/dove/Tennessee/4/75. In a specific embodiment, an APMV-7 that is used in a method of treating cancer described herein is and APMV-7 with a genome that has 80%, 85%, 90%, 95% or higher percent identity to the genome of APMV-7/dove/Tennessee/4/75.

In a specific embodiment, an APMV-2 strain is used in a method for treating cancer described herein. In another embodiment, an APMV-2 strain that is naturally occurring is used in a method of treating cancer described herein. In a preferred embodiment, an APMV-2 strain that is naturally occurring and has an intracerebral pathogenicity index in day-old chicks of the Gallus gallus species of less than 0.7 is used in a method of treating cancer described herein. In a specific embodiment, an APMV-2 that is used in a method of treating cancer described herein is APMV-2 Chicken/California/Yucaipa/1956. See, e.g., GenBank No. EU338414.1 for the complete genomic cDNA sequence of APMV-2 Chicken/California/Yucaipa/1956. In a specific embodiment, an APMV-2 that is used in a method of treating cancer described herein is and APMV-2 with a genome that has 80%, 85%, 90%, 95% or higher percent identity to the genome of APMV-2 Chicken/California/Yucaipa/1956.

In a specific embodiment, an APMV-3 strain is used in a method for treating cancer described herein. In another embodiment, an APMV-3 strain that is naturally occurring is used in a method of treating cancer described herein. In a preferred embodiment, an APMV-3 strain that is naturally occurring and has an intracerebral pathogenicity index in day-old chicks of the Gallus gallus species of less than 0.7 is used in a method of treating cancer described herein. In a specific embodiment, the APMV-3 that is used in a method of treating cancer described herein is APMV-3 turkey/Wisconsin/68. See, e.g., GenBank No. EU782025.1 for the complete genomic cDNA sequence of APMV-3 turkey/Wisconsin/68. In a specific embodiment, an APMV-3 that is used in a method of treating cancer described herein is and APMV-3 with a genome that has 80%, 85%, 90%, 95% or higher percent identity to the genome of APMV-3 turkey/Wisconsin/68.

In a specific embodiment, an APMV-5 strain is used in a method for treating cancer described herein. In another embodiment, an APMV-5 strain that is naturally occurring is used in a method of treating cancer described herein. In a preferred embodiment, an APMV-5 strain that is naturally occurring and has an intracerebral pathogenicity index in day-old chicks of the Gallus gallus species of less than 0.7 is used in a method of treating cancer described herein. See, e.g., NCBI Reference Sequence NC 025361.1 for the complete genomic cDNA sequence of an APMV-5.

In a specific embodiment, an APMV-6 strain is used in a method for treating cancer described herein. In another embodiment, an APMV-6 strain that is naturally occurring is used in a method of treating cancer described herein. In a preferred embodiment, an APMV-6 strain that is naturally occurring and has an intracerebral pathogenicity index in day-old chicks of the Gallus gallus species of less than 0.7 is used in a method of treating cancer described herein. In a specific embodiment, an APMV-6 that is used in a method of treating cancer described herein is APMV-6/duck/Hong Kong/18/199/77. See, e.g., GenBank No. EU622637.2 for the complete genomic cDNA sequence of APMV-6/duck/Hong Kong/18/199/77. In a specific embodiment, an APMV-6 that is used in a method of treating cancer described herein is an APMV-6 with a genome that has 80%, 85%, 90%, 95% or higher percent identity to the genome of APMV-6/duck/Hong Kong/18/199/77.

In a specific embodiment, an APMV-9 strain is used in a method for treating cancer described herein. In another embodiment, an APMV-9 strain that is naturally occurring is used in a method of treating cancer described herein. In a preferred embodiment, an APMV-9 strain that is naturally occurring and has an intracerebral pathogenicity index in day-old chicks of the Gallus gallus species of less than 0.7 is used in a method of treating cancer described herein. In a specific embodiment, an APMV-9 that is used in a method of treating cancer described herein is APMV-9 duck/New York/22/1978. See, e.g., GenBank No. NC 025390.1 for the complete genomic cDNA sequence of APMV-9 duck/New York/22/1978. In a specific embodiment, an APMV-9 that is used in a method of treating cancer described herein is an APMV-9 with a genome that has 80%, 85%, 90%, 95% or higher percent identity to the genome of APMV-9 duck/New York/22/1978.

In certain embodiments, an APMV (e.g., AMPV-1 or APMV-4) for use in a method for treating cancer described herein may be engineered to express a heterologous protein. For example, an APMV (e.g., AMPV-1 or APMV-4) may be engineered to express a checkpoint inhibitor (e.g., an antibody that specifically binds to PD-1 and blocks binding of PD-1 to PDL1, PDL2 or both, such as pembrolizumab or nivolumab; an antibody that specifically binds to PDL1 and blocks binding of PDL1 to PD1, CD80 or both, such as atezolizumab, durvalumab, or cemiplimab; an antibody that specifically binds to CLTA-4 and block the interaction of CTLA-4 with its ligands B7.1 and B7.2, such as ipilimumab or tremelimumab; and an antibody that specifically binds to TIM3); a cytokine (e.g., IL-2, IL-12, IL-15, IFN alpha/beta, 41-BB, CD40L, Flt3L, CCL3, CCL5, GM-CSF, etc.); an agonist of a co-stimulatory receptor (e.g., an agonist of ICOS, ICOS-L, OX40, OX40L, etc.); or a cancer antigen (e.g., a tumor associated antigen). See, e.g., Section 5.7.2 for examples of checkpoint inhibitors and agonists of co-stimulatory receptors. In other embodiments, an APMV (e.g., AMPV-1 or APMV-4) for use in a method for treating cancer described herein is not engineered to express a checkpoint inhibitor, a cytokine, an agonist of a co-stimulatory receptor, or a cancer antigen.

In certain embodiments, an APMV (e.g., AMPV-1 or APMV-4) for use in a method for treating cancer described herein is not engineered to express a heterologous protein. In some embodiments, an APMV for use in a method for treating cancer is not engineered to express a cancer antigen (e.g., a tumor associated antigen). In certain embodiments, an AMPV (e.g., AMPV-1 or APMV-4) for use in a method for treating cancer is not engineered to express a heterologous viral antigen. In some embodiments, an APMV (e.g., AMPV-1 or APMV-4) for use in a method for treating cancer is not engineered to express a bacterial antigen, a fungal antigen, a protozoal antigen, or a helminth antigen.

### 5.2 VEGF-C, VEGF-D and Other VEGFR-3 Activating Agents

In one aspect, provided herein are VEGFR-3-activating agents. In a specific embodiment, an agent is a VEGFR-3-activating agent if it induces or enhances phosphorylation of the VEGFR-3 and induces or enhances downstream signaling events, such as, e.g., phosphorylation of serine/threonine kinases, such as, e.g., AKT, ERK1/2 or STAT3. VEGFR-3 is expressed by several immune cell types, including macrophages, blood endothelial cells, and other myeloid cells. A VEGFR-3-activating agent may promote one, two or all of the following by cells expressing VEGFR-3 (e.g., macrophages, blood endothelial cells, or other myeloid cells): (1) proliferation, (2) migration, and (3) survival. In a particular, embodiment, a VEGFR-3-activating agent promotes lymphatic endothelial cell (LEC) proliferation, migration and survival. In another embodiment, a VEGFR-3-activating agent promotes macrophage activation, polarization, proliferation, migration and/or survival. In another embodiment, a VEGFR-3-activating agent promotes myeloid cell activation, proliferation, migration and/or survival. A VEGFR-3-activating agent may be a VEGF-C agent or a VEGF-D agent. In one embodiment, a VEGFR-3-activating agent is a recombinant VEGF-C protein or a recombinant VEGF-D protein. In another embodiment, a VEGFR-3-activating agent is a nucleic acid sequence comprising a nucleotide sequence encoding a recombinant VEGF-C protein or a recombinant VEGF-D protein. The recombinant VEGF-C protein or recombinant VEGF-D protein may be derivative of naturally occurring forms of VEGF-C or VEGF-D, respectively. See this section below for examples of VEGF-C proteins, VEGF-D proteins, nucleic acid sequences encoding a VEGF-C protein, nucleic acid sequences encoding a VEGF-D protein, VEGF-C derivatives, and VEGF-D derivatives. See Table 3 below for exemplary VEGF-C and VEGF-D nucleotide and amino acid sequences.

### VEGF-C Agent

In a specific embodiment, provided herein are vascular endothelial growth factor-C (VEGF-C) agents. In a specific embodiment, a VEGF-C agent is any agent that induces or enhances the expression, one or more functions, or both of VEGF-C. A VEGF-C agent may be a VEGF-C protein or derivative thereof, or a nucleic acid sequence encoding a VEGF-C protein or derivative thereof. In certain embodiments, a VEGF-C agent is conjugated, fused or linked to an antigen (e.g., bacterial antigen, a viral antigen, a fungal antigen, a protozoal antigen, a helminth antigen or a cancer antigen). In other embodiments, a VEGF-C agent is not conjugated, fused or linked to an antigen (e.g., bacterial antigen, a viral antigen, a fungal antigen, a protozoal antigen, a helminth antigen or a cancer antigen).

The terms “Vascular endothelial growth factor-C” and “VEGF-C” include any VEGF-C known to those of skill in the art. In a specific embodiment, VEGF-C refers to any naturally occurring form of VEGF-C. In some embodiments, VEGF-C refers to a derivative of a naturally occurring form of VEGF-C. In certain embodiments, the VEGF-C may be human, dog, cat, horse, pig, or cow VEGF-C. In a specific embodiment, the VEGF-C is human VEGF-C. GenBank™ accession number NM_005429.5 or Uniprot: P49767 provides an exemplary human VEGF-C nucleic acid sequence. After translation, the VEGF-C polypeptide typically consists of 3 domains, a central VEGF homology domain, an N-terminal domain and a C-terminal domain. GenBank™ accession number NM_005429.5 and Uniprot: P49767 provide an exemplary human VEGF-C amino acid sequence. In specific embodiments, the VEGF-C proteins are modified by post-translational processing such as signal peptide cleavage, disulfide bond formation, glycosylation (e.g., N-linked glycosylation), protease cleavage and lipid modification (e.g., S-palmitoylation). In some embodiments, the VEGF-C protein includes a signal sequence. For example, VEGF-C may undergo proteolytic maturation which includes the formation of an antiparallel homodimer linked by disulfide bonds and cleavage. Generally, the mature form of VEGF-C is composed of mostly two VEGF homology domains bound by non-covalent interactions. In certain embodiments, the VEGF-C protein does not include a signal sequence. The signal sequence can be the naturally occurring signal peptide sequence or a variant thereof. In some embodiments, the signal peptide is heterologous to a VEGF-C signal peptide (e.g., the signal sequence set forth in SEQ ID NO: 28 or 26).

In specific embodiments, a VEGF-C agent comprises or consists of a nucleotide sequence encoding VEGF-C. In certain embodiments, a VEGF-C agent may be a nucleic acid sequence comprising a nucleotide sequence, such as set forth in any one of SEQ ID NOs: 1-18, 29-40, 49, or 50. In a specific embodiment, a VEGF-C agent encodes human VEGF-C. In a specific embodiment, human VEGF-C comprises the amino acid sequence set forth in any one of SEQ ID NOs: 41-46. Given the degeneracy of the nucleic acid code, there are a number of different nucleic acid sequences that may encode the same VEGF-C protein. A nucleic acid sequence may encode precursor VEGF-C, pro-VEGF-C-ΔC, or mature VEGF-C (VEGF-CΔNΔC). In specific embodiments, a VEGF-C agent comprises or consists of VEGF-C protein. The VEGF-C protein may be precursor VEGF-C, pro-VEGF-C-ΔC, or mature VEGF-C (VEGF-CΔNΔC). In specific embodiments, a VEGF-C agent is a dimeric, secreted protein. In one embodiment, a VEGF-C agent comprises a full-length form of VEGF-C. In other embodiments, a VEGF-C agent comprises an unprocessed form of VEGF-C. In another embodiment, a VEGF-C agent comprises a pro-VEGF-C, which consists of two polypeptides. In another embodiment, a VEGF-C agent comprises the mature, full processed form of VEGF-C. In specific embodiments, a VEGF-C agent is one described in the Examples, infra. In some embodiments, a VEGF-C agent is a proteinaceous molecule, such as a protein encoded by the nucleotide sequence set forth in any one of SEQ ID NOs: 1-18, 29-40, 49, or 50 or a protein comprising the amino acid sequence set forth in any one of SEQ ID NOs: 19-24, 41-46, 51, or 52. In a specific embodiment, a VEGF-C agent comprises human VEGF-C. In a specific embodiment, human VEGF-C comprises the amino acid sequence set forth in any one of SEQ ID NOs: 41-46. Given the degeneracy of the nucleic acid code, there are a number of different nucleic acid sequences that may encode the same VEGF-C protein. In a specific embodiment, a VEGF-C agent is encoded by a nucleic acid sequence comprising the nucleotide sequence set forth in any one of SEQ ID NOs: 1-18, 29-40, 49, or 50. In a specific embodiment, a VEGF-C agent is encoded by a nucleic acid comprising the nucleotide sequence set forth in any one of SEQ ID NOs: 29-40. In certain embodiments, the VEGF-C agent further encodes a heterologous signal peptide, such as, e.g., set forth in SEQ ID NO: 25 or 27).

In a specific embodiment, a nucleic acid sequence comprising the nucleotide sequence encoding a VEGF-C agent (e.g., human VEGF-C) is codon optimized. See, e.g., Section 5.3.2.1, infra, for a discussion regarding codon optimization. In a specific embodiment, the nucleic acid sequence encoding a VEGF-C agent (e.g., human VEGF-C) comprises the nucleotide sequence set forth in any one of SEQ ID NOs: 7-21 or 35-40. In certain embodiments, a nucleic acid sequence comprising a nucleotide sequence encoding a VEGF-C agent, such as set forth in any one of SEQ ID NOs: 7-21 or 35-40, further comprises one, two, or more of the following: a regulatory sequence (e.g., a promoter, an enhancer, or both), Kozak sequences and restriction sites to facilitate cloning.

In a specific embodiment, a VEGF-C agent comprises murine VEGF-C. In another specific embodiment, murine VEGF-C comprises the amino acid sequence set forth in any one of SEQ ID NOs: 19-24. In another specific embodiment, the murine VEGF-C is encoded by a nucleic acid comprising the nucleotide sequence set forth in any one of SEQ ID NOs: 1-18. In another specific embodiment, a VEGF-C agent comprises canine VEGF-C. In another specific embodiment, canine VEGF-C comprises the amino acid sequence set forth in SEQ ID NO: 51 or 52. In another specific embodiment, a nucleic acid sequence comprising canine VEGF-C agent comprises the nucleotide sequence set forth in SEQ ID NO: 49 or 50.

In another specific embodiment, a VEGF-C agent is encoded by a nucleic acid sequence comprising the nucleotide sequence set forth in SEQ ID NO: 29, 32, or 35. In another embodiment, a VEGF-C agents comprises the nucleotide sequence set forth in SEQ ID NO: 29, 32, or 35.

In a specific embodiment, a nucleotide sequence or nucleic acid sequence encoding a VEGF-C agent may be a DNA molecule (e.g., cDNA or genomic DNA), an RNA molecule (e.g., mRNA), a combination of DNA and RNA molecule and a hybrid DNA/RNA molecule. In some embodiments, a nucleotide sequence or nucleic acid sequence encoding a VEGF-C agent may comprise analogs of DNA or RNA molecules. Such analogs can be generated using, for example, nucleotide analogs, which include, but are not limited to, inosine, methylcytosine, pseudouridine, or tritylated bases. Such analogs can also comprise DNA or RNA molecules comprising modified backbones that lend beneficial attributes to the molecules such as, for example, nuclease resistance or an increased ability to cross cellular membranes. The nucleic acid or nucleotide sequences can be single-stranded, double-stranded, may contain both single-stranded and double-stranded portions, and may contain triple-stranded portions. In a specific embodiment, a nucleotide sequence or nucleic acid sequence is an mRNA. In another specific embodiment, a nucleotide sequence or nucleic acid sequence is an mRNA sequence which includes nucleotide analogs (e.g., methylcytosine or pseudouridine).

In certain embodiments, a VEGF-C agent is a VEGF-C derivative. In a specific embodiment, a VEGF-C derivative has at least 70%, 75%, 80%, 85%, 90%, 95%, 96%, 98%, or 99% amino acid sequence identity to a VEGF-C known to those of skill in the art (e.g., any one of SEQ ID NOs: 19-24, 41-46, 51, or 52). In another specific embodiment, a VEGF-C derivative has at least 85%, 90%, 95%, 96%, 98%, or 99% amino acid sequence identity to a VEGF-C known to those of skill in the art (e.g., any one of SEQ ID NOs: 19-24, 41-46, 51, or 52). In another specific embodiment, a VEGF-C derivative has at least 90%, 95%, 96%, 98%, or 99% amino acid sequence identity to a VEGF-C known to those of skill in the art (e.g., any one of SEQ ID NOs: 19-24, 41-46, 51, or 52). In another specific embodiment, a VEGF-C derivative has at least 95%, 96%, 98%, or 99% amino acid sequence identity to a VEGF-C known to those of skill in the art (e.g., any one of SEQ ID NOs 19-24, 41-46, 51, or 52). In another embodiment, a VEGF-C derivative has at least 85%, 90%, 95%, 96%, 98% or 99% identity to native human VEGF-C (e.g., SEQ ID NO:41) or a fragment thereof (e.g., a fragment comprising the VEGF homology domain). In another embodiment, a VEGF-C derivative has at least 85%, 90%, 95%, 96%, 98% or 99% identity to native mature VEGF-C (e.g., SEQ ID NO: 44). In another embodiment, a VEGF-C derivative comprises a VEGF homology domain, wherein the VEGF homology domain has at least 85%, 90%, 95%, 96%, 98% or 99% identity to the VEGF homology domain of native VEGF-C (e.g., human VEGF-C). Methods/techniques known in the art may be used to determine sequence identity (see, e.g., “Best Fit” or “Gap” program of the Sequence Analysis Software Package, version 10; Genetics Computer Group, Inc.). In a specific embodiment, a VEGF-C derivative comprises deleted forms of a known VEGF-C (e.g., human VEGF-C), wherein up to about 20, 15, 10, 9, 8, 7, 6, 5, 4, 3, 2 or 1 amino acid residues are deleted from the known VEGF-C (e.g., human VEGF-C). Also provided herein are VEGF-C derivatives comprising deleted forms of a known VEGF-C, wherein about 1-3, 3-5, 5-7, 7-10, 10-15, or 15-20 amino acid residues are deleted from the known VEGF-C (e.g., human VEGF-C). Further provided herein are VEGF-C derivatives comprising altered forms of a known VEGF-C (e.g., human VEGF-C), wherein up to about 20, 15, 10, 9, 8, 7, 6, 5, 4, 3, 2 or 1 amino acid residues of the known VEGF-C are substituted (e.g., conservatively substituted) with other amino acids. In a specific embodiment, the known VEGF-C is human VEGF-C, such as, e.g., provided in GenBank™ accession number NM_005429.5, Uniprot: P49767, or Uniprot Q6FH59, or SEQ ID NO: 41 or 44. In some embodiments, the known VEGF-C is canine VEGF-C, such as, e.g., provided in GenBank™ accession numbers XM_S40047.6 and XM_02543044, or SEQ ID NO: 51 or 52. In some embodiments, a VEGF-C derivative comprises up to about 20, 15, 10, 9, 8, 7, 6, 5, 4, 3, 2 or 1 conservatively substituted amino acids. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophylic (Cys, Ser, Thr), acidic (Asp, Glu), basic (Asn, Gln, His, Lys, Arg), conformation disruptors (Gly, Pro) and aromatic (Trp, Tyr, Phe).

In a specific embodiment, a VEGF-C derivative is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a native VEGF-C (e.g., human VEGF-C). In a specific embodiment, a VEGF-C derivative is at least 90%, 95%, 98%, or 99% or is 90% to 95%, 90% to 99%, 95% to 98%, or 95% to 99% identical (e.g., sequence identity) to a native VEGF-C (e.g., human VEGF-C). In another specific embodiment, a VEGF-C derivative is a polypeptide encoded by a nucleic acid sequence that is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a nucleic acid sequence encoding a native VEGF-C. In another specific embodiment, a VEGF-C is a polypeptide encoded by a nucleic acid sequence that is at least 90%, 95%, 98%, or 99% or is 90% to 95%, 90% to 99%, 95% to 98%, or 95% to 99% identical (e.g., sequence identity) to a nucleic acid sequence encoding a native VEGF-C. In a specific embodiment, the native VEGF-C is human VEGF-C, such as, e.g., provided in GenBank™ accession number NM_005429.5, Uniprot: P49767, or Uniprot Q6FH59, or SEQ ID NO: 41 or 44. In some embodiments, the native VEGF-C is canine VEGF-C, such as, e.g., provided in GenBank™ accession numbers XM_S40047.6 and XM_02543044, or SEQ ID NO: 51 or 52. In another specific embodiment, a VEGF-C derivative contains 1, 2, 3, 4, 5, 6, 7, 8, 9, 10, 11, 12, 13, 14, 15, 16, 17, 18, 19, 20 or more, or 2 to 5, 2 to 10, 5 to 10, 5 to 15, 5 to 20, 10 to 15, or 15 to 20 amino acid mutations (i.e., additions, deletions, substitutions or any combination thereof) relative to a native VEGF-C (e.g., human VEGF-C). In another specific embodiment, a VEGF-C derivative is a polypeptide encoded by nucleic acid sequence that can hybridize under high, moderate or typical stringency hybridization conditions to a nucleic acid sequence encoding a native VEGF-C (e.g., human VEGF-C). Hybridization conditions are known to one of skill in the art (see, e.g., U.S. Patent Application No. 2005/0048549 at, e.g., paragraphs 72 and 73). In another specific embodiment, a VEGF-C derivative is a polypeptide encoded by a nucleic acid sequence that can hybridize under high, moderate or typical stringency hybridization conditions to a nucleic acid sequence encoding a fragment of a native VEGF-C (e.g., human VEGF-C) of at least 10 contiguous amino acids, at least 12 contiguous amino acids, at least 15 contiguous amino acids, at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, at least 150 contiguous amino acids, or 10 to 20, 20 to 50, 25 to 75, 25 to 100, 25 to 150, 50 to 75, 50 to 100, 75 to 100, 50 to 150, 75 to 150, 100 to 150, or 100 to 200 contiguous amino acids. In another specific embodiment, a VEGF-C derivative is a fragment of a native VEGF-C (e.g., human VEGF-C). In another specific embodiment, a VEGF-C derivative comprises a fragment of human VEGF-C (e.g., a fragment of SEQ ID NO: 41 or 44). In another specific embodiment, a VEGF-C derivative comprises a fragment of a human VEGF-C (e.g., SEQ ID NO: 41 or 44), wherein the fragment comprises the VEGF homology domain. In a specific embodiment, a VEGF-D derivative is a fragment of a native VEGF-D (e.g., a human VEGF-D) that comprises the VEGF homology domain. In a specific embodiment, a fragment of native VEGF-C retains the ability to bind to VEGFR-3, induces phosphorylation of VEGFR-3 and induces downstream signaling events, such as, e.g., phosphorylation of serine/threonine kinases, such as, e.g., AKT, ERK 1/2, or Stat 3. VEGF-C derivatives also include polypeptides that comprise the amino acid sequence of a naturally occurring mature form of VEGF-C and a heterologous amino acid sequence. VEGF-C derivatives also include polypeptides that comprise the amino acid sequence of a naturally occurring mature form of VEGF-C and a heterologous signal peptide amino acid sequence (e.g., the signal peptide set forth in SEQ ID NO: 28 or 26). In some embodiments, the VEGF-C derivative comprises (i) a polypeptide that comprises the amino acid sequence of a naturally occurring mature form of VEGF-C and (ii) a heterologous amino acid sequence (e.g., a heterologous signal peptide, such as, e.g., the signal peptide for Gaussia luciferase (e.g., SEQ ID NO: 28 or 26)). In addition, VEGF-C derivatives include polypeptides that have been chemically modified by, e.g., glycosylation, acetylation, pegylation, phosphorylation, amidation, derivitization by known protecting/blocking groups, proteolytic cleavage, linkage to a cellular ligand or other protein moiety, etc. Further, VEGF-C derivatives include polypeptides comprising one or more non-classical amino acids.

In a specific embodiment, a VEGF-C derivative comprises a VEGF-C amino acid sequence (e.g., SEQ ID NO: 41) with an amino acid substitution of Cys156Ser or Cys137Ala. See, e.g., Kajiya K et al., 2009, J Invest Dermatol. 129:1292-8 and Leppanen et al., 2010, Proc Natl Acad Sci USA. 107(6):2425-30 for a description of such forms of VEGF-C. In certain embodiments, a VEGF-C derivative comprises the nucleotide sequence of SEQ ID NO: 30, 31, 33, 34, 36, 37, 39, or 40. In some embodiments, a VEGF-C derivative is mature VEGF-C Cys156Ser (e.g., SEQ ID NO: 45). In other embodiments, a VEGF-C derivative is human VEGF-C Cys137Ala (e.g., SEQ ID NO: 46).

In a specific embodiment, a VEGF-C derivative binds to VEGFR-3 but not VEGFR-2. In a specific embodiment, a VEGF-C derivative binds to VEGFR-3 and induces phosphorylation of VEGFR-3 and activates downstream signaling, including but not limited to phosphorylation of serine/threonine kinases, such as AKT, ERT1/2 and Stat3. In specific embodiments, the VEGF-C derivative retains one, two, or more, or all of the functions of the native VEGF-C (e.g., human VEGF-C) from which it was derived. Examples of functions of VEGF-C include lymphangiogenesis, lymphatic endothelial proliferation and migration, lymphatic permeability and contractility, angiogenesis, macrophage recruitment and immunomodulation. Tests for determining whether or not a VEGF-C derivative retains one or more functions of the native VEGF-C (e.g., human VEGF-C) from which it was derived are known to one of skill in the art and examples are provided herein.

In a specific embodiment, a VEGF-C agent comprises the nucleotide sequence set forth in any one of SEQ ID NOs: 1-18, 29-40, 49, or 50 or is encoded by the nucleotide sequence set forth in any one of SEQ ID NOs: 1-18, 29-40, 49, or 50. In a specific embodiment, a VEGF-C agent comprises the amino acid sequence set forth in any one of SEQ ID NOs: 19-24, 41-46, 51, or 52. Examples of VEGF-C sequences which may be used in accordance with the methods described herein are provided in Table 3, infra.

Techniques known to one of skill in the art may be used to produce a VEGF-C agent. For example, standard methods in molecular biology are described Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4). In a specific embodiment, a VEGFR-3-activating agent is recombinantly produced. In another specific embodiment, a VEGF-C agent is recombinantly produced.

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). In a specific embodiment, a VEGFR-3 activating agent is isolated. In another specific embodiment, a VEGF-C agent is isolated.

In a specific embodiment, a protein is isolated when substantially free of contaminating materials from the natural source, e.g., soil particles, minerals, chemicals from the environment, and/or cellular materials from the natural source, such as but not limited to cell debris, cell wall materials, membranes, organelles, the bulk of the nucleic acids, carbohydrates, proteins, and/or lipids present in cells. Thus, a protein that is isolated includes preparations of a polypeptide having less than about 30%, 20%, 10%, 5%, 2%, or 1% (by dry weight) of cellular materials and/or contaminating materials. In some embodiments, a chemically synthesized polypeptide is isolated when substantially free of chemical precursors or other chemicals which are involved in the syntheses of the polypeptide. The term “substantially free of chemical precursors or other chemicals” includes preparations in which the amino acid sequence is separated from chemical precursors or other chemicals which are involved in the synthesis of the amino acid sequence. In specific embodiments, such preparations of the amino acid sequence have less than about 50%, 30%, 20%, 10%, 5% (by dry weight) of chemical precursors or compounds other than the amino acid sequence of interest.

In certain embodiments, an “isolated” nucleic acid sequence refers to a nucleic acid molecule which is separated from other nucleic acid molecules which are present in the natural source of the nucleic acid. In other words, the isolated nucleic acid sequence can comprise heterologous nucleic acids that are not associated with it in nature. In other embodiments, an “isolated” nucleic acid sequence, such as a cDNA or RNA sequence, can be substantially free of other cellular material, or culture medium when produced by recombinant techniques, or substantially free of chemical precursors or other chemicals when chemically synthesized. The term “substantially free of cellular material” includes preparations of nucleic acid sequences in which the nucleic acid sequence is separated from cellular components of the cells from which it is isolated or recombinantly produced. Thus, nucleic acid sequence that is substantially free of cellular material includes preparations of nucleic acid sequence having less than about 30%, 20%, 10%, or 5% (by dry weight) of other nucleic acids. The term “substantially free of culture medium” includes preparations of nucleic acid sequence in which the culture medium represents less than about 50%, 20%, 10%, or 5% of the volume of the preparation. The term “substantially free of chemical precursors or other chemicals” includes preparations in which the nucleic acid sequence is separated from chemical precursors or other chemicals which are involved in the synthesis of the nucleic acid sequence. In specific embodiments, such preparations of the nucleic acid sequence have less than about 50%, 30%, 20%, 10%, 5% (by dry weight) of chemical precursors or compounds other than the nucleic acid sequence of interest.

### VEGF-D Agent

In a specific embodiment, provided herein are vascular endothelial growth factor-D (VEGF-D) agents. In a specific embodiment, a VEGF-D agent is any agent that induces or enhances the expression, one or more functions, or both of VEGF-D. A VEGF-D agent may be a VEGF-D protein or derivative thereof, or a nucleic acid sequence encoding a VEGF-D protein or derivative thereof. In certain embodiments, a VEGF-D agent is conjugated, fused or linked to an antigen (e.g., bacterial antigen, a viral antigen, a fungal antigen, a protozoal antigen, a helminth antigen or a cancer antigen). In other embodiments, a VEGF-D agent is not conjugated, fused or linked to an antigen (e.g., bacterial antigen, a viral antigen, a fungal antigen, a protozoal antigen, a helminth antigen or a cancer antigen).

The terms “Vascular endothelial growth factor-D” and “VEGF-D” include any VEGF-D known to those of skill in the art. In a specific embodiment, VEFG-D refers to any naturally occurring form of VEGF-D. In some embodiments, VEGF-D refers to a derivative of a naturally occurring form of VEGF-D. In certain embodiments, the VEGF-D may be human, dog, cat, horse, pig, or cow VEGF-D. In a specific embodiment, the VEGF-D is human VEGF-D. Uniprot O43915 provides an exemplary human VEGF-D nucleic acid sequence. After translation, the VEGF-D polypeptide generally consists of 3 domains, a central VEGF homology domain, an N-terminal domain and a C-terminal domain. Uniprot O43915 provides an exemplary human VEGF-D amino acid sequence. In specific embodiments, the VEGF-D proteins are modified by post-translational processing such as signal peptide cleavage, disulfide bond formation, glycosylation (e.g., N-linked glycosylation), protease cleavage and lipid modification (e.g., S-palmitoylation). For example, VEGF-D may undergo proteolytic maturation which includes the formation of an antiparallel homodimer linked by disulfide bonds and cleavage. Generally, the mature form of VEGF-D is composed of mostly two VEGF homology domains bound by non-covalent interactions. In some embodiments, VEGF-D protein includes a signal sequence. In other embodiments, VEGF-D protein does not include a signal sequence. The signal sequence can be the naturally occurring signal peptide sequence or a variant thereof. In some embodiments, the signal peptide is a VEGF-D signal peptide. In some embodiments, the signal peptide is heterologous to a VEGF-D signal peptide (e.g., a signal peptide set forth in SEQ ID NO: 28 or 26).

In specific embodiments, a VEGF-D agent comprises or consists of a nucleotide sequence encoding VEGF-D. In certain embodiments, a VEGF-D agent may be a nucleic acid sequence comprising a nucleotide sequence, such as set forth in any one of SEQ ID NOs: 96-98. In a specific embodiment, a VEGF-D agent encodes human VEGF-D. In a specific embodiment, human VEGF-D comprises the amino acid sequence set forth in SEQ ID NO: 101-104. Given the degeneracy of the nucleic acid code, there are a number of different nucleic acid sequences that may encode the same VEGF-D protein. A nucleic acid sequence may encode precursor VEGF-D, pro-VEGF-D-ΔC, or mature VEGF-D (VEGF-DΔNΔC).

In specific embodiments, a VEGF-D agent comprises or consists of VEGF-D protein. The VEGF-D protein may be precursor VEGF-D, pro-VEGF-D-ΔC, or mature VEGF-D (VEGF-DΔNΔC). In specific embodiments, a VEGF-D agent is a dimeric, secreted protein. In one embodiment, a VEGF-D agent comprises a pro-VEGF-D, which consists of two polypeptides. In another embodiment, a VEGF-D agent comprises the mature, full processed form of VEGF-D. In certain embodiments, a VEGF-D agent is a proteinaceous molecule, such as a protein encoded by the nucleotide sequence set forth in any one of SEQ ID NOs: 96-98, or a protein comprising the amino acid sequence set forth in any one of SEQ ID NO: 99-104. In a specific embodiment, a VEGF-D agent comprises human VEGF-D. In a specific embodiment, human VEGF-D comprises the amino acid sequence set forth in SEQ ID NO: 101-104. In another specific embodiment, human VEGF-D is encoded by a nucleic acid sequence comprising the nucleotide sequence set forth in SEQ ID NO: 96. Given the degeneracy of the nucleic acid code, there are a number of different nucleic acid sequences that may encode the same VEGF-D protein. In a specific embodiment, a VEGF-D agent is encoded by a nucleic acid sequence comprising the nucleotide sequence set forth in any one of SEQ ID NOs: 96-98.

In a specific embodiment, a VEGF-D agent comprises canine VEGF-D. In a specific embodiment, canine VEGF-D comprises the amino acid sequence set forth in SEQ ID NO: 99 or 100. In a specific embodiment, canine VEGF-D is encoded by a nucleic acid sequence comprising the nucleotide sequence set forth in SEQ ID NO: 97 or 98.

In a specific embodiment, a VEGF-D agent comprises human VEGF-D. In a specific embodiment, human VEGF-D comprises the amino acid sequence set forth in SEQ ID NO: 101-104. In a specific embodiment, human VEGF-D is encoded by a nucleic acid sequence comprising the nucleotide sequence set forth in SEQ ID NO: 96.

In a specific embodiment, a nucleic acid sequence comprising the nucleotide sequence encoding a VEGF-D agent (e.g., human VEGF-D) is codon optimized. See, e.g., Section 5.3.2.1, infra, for a discussion regarding codon optimization. In certain embodiments, a nucleic acid sequence comprising a nucleotide sequence encoding a VEGF-D protein, such as set forth in any one of SEQ ID NOs: 96-98, further comprises one, two, or more of the following: a regulatory sequence (e.g., a promoter, an enhancer, or both), Kozak sequences and restriction sites to facilitate cloning.

In a specific embodiment, a nucleotide sequence or nucleic acid sequence encoding a VEGF-D agent may be a DNA molecule (e.g., cDNA or genomic DNA), an RNA molecule (e.g., mRNA), a combination of DNA and RNA molecule and a hybrid DNA/RNA molecule. In some embodiments, a nucleotide sequence or nucleic acid sequence encoding a VEGF-D agent may comprise analogs of DNA or RNA molecules. Such analogs can be generated using, for example, nucleotide analogs, which include, but are not limited to, inosine methylcytosine, pseudouridine, or tritylated bases. Such analogs can also comprise DNA or RNA molecules comprising modified backbones that lend beneficial attributes to the molecules such as, for example, nuclease resistance or an increased ability to cross cellular membranes. The nucleic acid or nucleotide sequences can be single-stranded, double-stranded, may contain both single-stranded and double-stranded portions, and may contain triple-stranded portions. In a specific embodiment, a nucleotide sequence or nucleic acid sequence is an mRNA. In another specific embodiment, a nucleotide sequence or nucleic acid sequence is an mRNA sequence which includes nucleotide analogs (e.g., methylcytosine or pseudouridine).

In certain embodiments, a VEGF-D agent is a VEGF-D derivative. In a specific embodiment, a VEGF-D derivative has at least 70%, 75%, 80%, 85%, 90%, 95%, 96%, 98%, or 99% amino acid sequence identity to a VEGF-D known to those of skill in the art (e.g., any one of SEQ ID NO: 99-104). In another specific embodiment, a VEGF-D derivative has at least 85%, 90%, 95%, 96%, 98%, or 99% amino acid sequence identity to a VEGF-D known to those of skill in the art (e.g., any one of SEQ ID NO: 99-104). In another specific embodiment, a VEGF-D derivative has at least 90%, 95%, 96%, 98%, or 99% amino acid sequence identity to a VEGF-D known to those of skill in the art (e.g., any one of SEQ ID NO: 99-104). In another specific embodiment, a VEGF-D derivative has at least 95%, 96%, 98%, or 99% amino acid sequence identity to a VEGF-D known to those of skill in the art (e.g., any one of SEQ ID NO: 99-104). In another embodiment, a VEGF-D derivative has at least 85%, 90%, 95%, 96%, 98% or 99% identity to native human VEGF-D (e.g., SEQ ID NO: 104) or a fragment thereof (e.g., a fragment comprising the VEGF homology domain). In another embodiment, a VEGF-D derivative has at least 85%, 90%, 95%, 96%, 98% or 99% identity to native mature VEGF-D (e.g., SEQ ID NO: 101). In another embodiment, a VEGF-D derivative comprises a VEGF homology domain, wherein the VEGF homology domain has at least 85%, 90%, 95%, 96%, 98% or 99% identity to the VEGF homology domain of native VEGF-D. Methods/techniques known in the art may be used to determine sequence identity (see, e.g., “Best Fit” or “Gap” program of the Sequence Analysis Software Package, version 10; Genetics Computer Group, Inc.). In a specific embodiment, a VEGF-D derivative comprises deleted forms of a known VEGF-D (e.g., human VEGF-D), wherein up to about 20, 15, 10, 9, 8, 7, 6, 5, 4, 3, 2 or 1 amino acid residues are deleted from the known VEGF-D (e.g., human VEGF-D). Also provided herein are VEGF-D derivatives comprising deleted forms of a known VEGF-D, wherein about 1-3, 3-5, 5-7, 7-10, 10-15, or 15-20 amino acid residues are deleted from the known VEGF-D (e.g., human VEGF-D). Further provided herein are VEGF-D derivatives comprising altered forms of a known VEGF-D (e.g., human VEGF-D), wherein up to about 20, 15, 10, 9, 8, 7, 6, 5, 4, 3, 2 or 1 amino acid residues of the known VEGF-D are substituted (e.g., conservatively substituted) with other amino acids. In a specific embodiment, the known VEGF-D is human VEGF-D, such as, e.g., provided in Uniprot O43915. In certain embodiments, the known VEGF-D, is canine VEGF-D, such, e.g., provided in GenBank™ accession number XM_548869.5 or XM_025437083. In some embodiments, a VEGF-D derivative comprises up to about 20, 15, 10, 9, 8, 7, 6, 5, 4, 3, 2 or 1 conservatively substituted amino acids. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophylic (Cys, Ser, Thr), acidic (Asp, Glu), basic (Asn, Gln, His, Lys, Arg), conformation disruptors (Gly, Pro) and aromatic (Trp, Tyr, Phe).

In a specific embodiment, a VEGF-D derivative is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a native VEGF-D (e.g., human VEGF-D). In a specific embodiment, a VEGF-D derivative is at least 90%, 95%, 98%, or 99% or is 90% to 95%, 90% to 99%, 95% to 98%, or 95% to 99% identical (e.g., sequence identity) to a native VEGF-D (e.g., human VEGF-D). In another specific embodiment, a VEGF-D derivative is a polypeptide encoded by a nucleic acid sequence that is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a nucleic acid sequence encoding a native VEGF-D. In another specific embodiment, a VEGF-D is a polypeptide encoded by a nucleic acid sequence that is at least 90%, 95%, 98%, or 99% or is 90% to 95%, 90% to 99%, 95% to 98%, or 95% to 99% identical (e.g., sequence identity) to a nucleic acid sequence encoding a native VEGF-D. In a specific embodiment, the native VEGF-D is human VEGF-D, such as, e.g., provided in Uniprot O43915. In other embodiments, the native VEGF-D is a canine VEGF-D, such as e.g., provided in GenBank™ accession numbers XM_548869.5 or XM_025437083. In another specific embodiment, a VEGF-D derivative contains 1, 2, 3, 4, 5, 6, 7, 8, 9, 10, 11, 12, 13, 14, 15, 16, 17, 18, 19, 20 or more, or 2 to 5, 2 to 10, 5 to 10, 5 to 15, 5 to 20, 10 to 15, or 15 to 20 amino acid mutations (i.e., additions, deletions, substitutions or any combination thereof) relative to a native VEGF-D (e.g., human VEGF-D). In another specific embodiment, a VEGF-D derivative is a polypeptide encoded by nucleic acid sequence that can hybridize under high, moderate or typical stringency hybridization conditions to a nucleic acid sequence encoding a native VEGF-D (e.g., human VEGF-D). Hybridization conditions are known to one of skill in the art (see, e.g., U.S. Patent Application No. 2005/0048549 at, e.g., paragraphs 72 and 73). In another specific embodiment, a VEGF-D derivative is a polypeptide encoded by a nucleic acid sequence that can hybridize under high, moderate or typical stringency hybridization conditions to a nucleic acid sequence encoding a fragment of a native VEGF-D (e.g., human VEGF-D) of at least 10 contiguous amino acids, at least 12 contiguous amino acids, at least 15 contiguous amino acids, at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, at least 150 contiguous amino acids, or 10 to 20, 20 to 50, 25 to 75, 25 to 100, 25 to 150, 50 to 75, 50 to 100, 75 to 100, 50 to 150, 75 to 150, 100 to 150, or 100 to 200 contiguous amino acids. In another specific embodiment, a VEGF-D derivative is a fragment of a native VEGF-D (e.g., human VEGF-D). In another specific embodiment, a VEGF-D derivative comprises a fragment of human VEGF-D (e.g., a fragment of any one of SEQ ID Nos: 100-104). In a specific embodiment, a VEGF-D derivative is a fragment of a native VEGF-D (e.g., a human VEGF-D) that comprises the VEGF homology domain. In another specific embodiment, a VEGF-D derivative comprises a fragment of a human VEGF-D (e.g., any one of SEQ ID NOs: 100-104), wherein the fragment comprises the VEGF homology domain. In a specific embodiment, a fragment of native VEGF-D retains the ability to bind to VEGFR-3, induces phosphorylation of VEGFR-3 and induces downstream signaling events, such as, e.g., phosphorylation of serine/threonine kinases, such as, e.g., AKT, ERK 1/2, or Stat 3.

VEGF-D derivatives also include polypeptides that comprise the amino acid sequence of a naturally occurring mature form of VEGF-D and a heterologous amino acid sequence. VEGF-D derivatives also include polypeptides that comprise the amino acid sequence of a naturally occurring mature form of VEGF-D and a heterologous signal peptide amino acid sequence. In some embodiments, the VEGF-D derivative comprises (i) a polypeptide that comprises the amino acid sequence of a naturally occurring mature form of VEGF-D and (ii) a heterologous peptide amino acid sequence (e.g., a heterologous signal peptide, such as, e.g., the signal peptide for Gaussia luciferase (e.g., SEQ ID NO: 28) or the signal peptide for IgG light chain signal peptide (e.g., SEQ ID NO: 26). In addition, VEGF-D derivatives include polypeptides that have been chemically modified by, e.g., glycosylation, acetylation, pegylation, phosphorylation, amidation, derivitization by known protecting/blocking groups, proteolytic cleavage, linkage to a cellular ligand or other protein moiety, etc. Further, VEGF-D derivatives include polypeptides comprising one or more non-classical amino acids.

In a specific embodiment, a VEGF-D derivative binds to VEGFR-3 and induces phosphorylation of VEGFR-3 and activates downstream signaling, e.g., phosphorylation of serine/threonine kinases, such as AKT, ERT1/2 or Stat3. In specific embodiments, the VEGF-D derivative retains one, two, or more, or all of the functions of the native VEGF-D (e.g., human VEGF-D) from which it was derived. Examples of functions of VEGF-D include lymphatic endothelial proliferation and migration, lymphatic permeability and contractility, angiogenesis, and remodeling of lymphatic and blood vessels. Tests for determining whether or not a VEGF-D derivative retains one or more functions of the native VEGF-D (e.g., human VEGF-D) from which it was derived are known to one of skill in the art and examples are provided herein. In a specific embodiment, a VEGF-D derivative binds to VEGFR-3 but not VEGFR-2/.

In a specific embodiment, a VEGF-D agent comprises the amino acid sequence set forth in any one of SEQ ID NOs: 99-104, or is encoded by the nucleotide sequence set forth in any one of SEQ ID NOs: 96-98. Examples of VEGF-D sequences which may be used in accordance with the methods described herein are provided in Table 3, infra.

Techniques known to one of skill in the art may be used to produce a VEGF-D agent. For example, standard methods in molecular biology are described Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4). In another specific embodiment, a VEGF-D agent is recombinantly produced.

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). In another specific embodiment, a VEGF-D agent is isolated.

### 5.3 Recombinant Oncolytic Viruses

In one aspect, provided herein are recombinant viruses that provided herein are recombinant viruses comprising a genome, wherein the genome comprises a transgene comprising a nucleotide sequence encoding a VEGFR-3 activating agent. In some embodiments, the virus can be any virus known in the art, including, e.g., an adeno-associated virus (“AAV”; e.g., AAV1-AAV9). In other embodiments, the virus is not an adeno-associate virus (e.g., is not AAV1-AAV9). In one aspect, provided herein are recombinant oncolytic viruses comprising a genome, wherein the genome comprises a transgene comprising a nucleotide sequence encoding a VEGFR-3-activating agent. In one embodiment, a VEGFR-3-activating agent is a VEGF-C protein or a VEGF-D protein. In another embodiment, a VEGFR-3-activating agent is a nucleic acid sequence comprising a nucleotide sequence encoding a VEGF-C protein or a VEGF-D protein. The VEGF-C protein or VEGF-D protein may be derivatives of VEGF-C or VEGF-D, respectively. See Section 5.2 and 5.3.2 for examples of VEGF-C proteins, VEGF-D proteins, nucleic acid sequences encoding a VEGF-C protein, and nucleic acid sequences encoding a VEGF-D protein, VEGF-C derivatives, and VEGF-D derivatives.

In another aspect, provided herein are recombinant oncolytic viruses comprising a genome, wherein the genome comprises a transgene comprising a nucleotide sequence encoding a VEGF-C agent, a VEGF-D agent, or both. In another aspect, provided herein are recombinant oncolytic viruses comprising a genome, wherein the genome comprises a transgene comprising a nucleotide sequence encoding a VEGF-C agent and a transgene comprising a nucleotide sequence encoding a VEGF-D agent. See, e.g., Section 5.3 and Section 6 for examples of transgenes which may be incorporated into the genome of an oncolytic virus. See e.g., section 5.1 and 6 for examples of viruses which may be engineered to encode/express a transgene. In some embodiments, recombinant oncolytic virus is a parovirus (e.g., an autonomous parvovirus), a myxoma virus, an avian paramyxovirus (e.g., Newcastle disease virus or APMV-4), a reovirus, or Seneca valley virus. In one embodiment, the recombinant oncolytic virus is wild-type parvovirus H1 (ParvOryx). In another embodiment, the recombinant oncolytic virus is Vesicular stomatitis virus. In another embodiment, the recombinant oncolytic virus is an avian paramyxovirus. See Sections 5.1.1 and 5.3 (including 5.3.1.1) regarding avian paramyxoviruses. In some embodiments, the recombinant oncolytic virus is a genetically engineered influenza virus, measles virus, poliovirus, vaccinia virus, poxvirus, picornavirus, alphavirus, retrovirus, rhabdovirus, reovirus, adenovirus, herpes simplex virus, or vesicular stomatitis virus. In a specific embodiment, such viruses are attenuated. In one embodiment, the recombinant oncolytic virus is an E1A/E1B-deleted adenovirus (ONYX015) (see, e.g., Cohen and Rudin, 2001, Curr. Opin. Investig. Drugs 2(12): 1770-1775, which is incorporated by reference in its entirety, for information regarding ONYX015. In another embodiment, the recombinant oncolytic virus is the adenovirus is H101, a conditionally replicative adenovirus, was generated by both E1B and E3 gene deletion (see, e.g., Kasuya et al., 2007, Curr Cancer Drug Targets. 7:123-125, which is incorporated by reference in its entirety, for information regarding H101). In another embodiment, the recombinant oncolytic virus is adenovirus known as Delta-24-RGD (DNX-2401). In another embodiment, the recombinant oncolytic virus is an attenuated influenza virus (e.g., an influenza virus comprising a truncated NS1 protein, such as described in U.S. Pat. Nos. 10,098,945; 8,057,803; 8,124,101; 8,137,676; 6,866,853; 6,669,943; 6,468,544; 8,137,676; and 9,387,240, each of which is incorporated herein by reference in its entirety). In another embodiment, the recombinant oncolytic virus is HSV1716 (Seprehvir®). In another embodiment, the recombinant oncolytic virus is G207. In another embodiment, the recombinant oncolytic virus is Pelareorep (Reolysin®).

In certain embodiments, a recombinant oncolytic viruses comprises a genome, wherein the genome comprises a first transgene comprising a nucleotide sequence encoding a VEGF-C agent, a VEGF-D agent, or both, and a second transgene comprising a nucleotide sequence encoding a heterologous protein. In some embodiments, a recombinant oncolytic viruses comprises a genome, wherein the genome comprises a first transgene comprising a nucleotide sequence encoding a VEGF-C agent, a second transgene comprising a nucleotide sequence encoding VEGF-D, and a third transgene comprising a nucleotide sequence encoding a heterologous protein. In certain aspects, provided herein is a combination of oncolytic viruses comprising two, three or more oncolytic viruses. In some embodiments, a first recombinant oncolytic virus comprises a first genome, wherein the first genome comprises a first transgene comprising a nucleotide sequence encoding a VEGF-C agent, a second recombinant oncolytic virus comprises a second genome, wherein the second genome comprises a second transgene comprising a nucleotide sequence encoding VEGF-D, and a third recombinant oncolytic virus comprises a third genome, wherein the third genome comprises a third transgene comprising a nucleotide sequence encoding a heterologous protein. In some embodiments, the combination of oncolytic viruses consists of the first and second oncolytic viruses. In some embodiments, the combination of oncolytic viruses consists of the first and third oncolytic viruses. In some embodiments, the combination of oncolytic viruses consists of the first, second and third oncolytic viruses. For example, an oncolytic virus may be engineered to express a checkpoint inhibitor (e.g., an antibody that specifically binds to PD-1 and blocks binding of PD-1 to PDL1, PDL2 or both, such as pembrolizumab or nivolumab; an antibody that specifically binds to PDL1 and blocks binding of PDL1 to PD1, CD80 or both, such as atezolizumab, durvalumab, or cemiplimab; and an antibody that specifically binds to CLTA-4 and block the interaction of CTLA-4 with its ligands B7.1 and B7.2, such as ipilimumab or tremelimumab and an antibody that specifically binds to TIM3); a cytokine (e.g., IL-2, IL-12, IL-15, IFN alpha/beta, 41-BB, CD40L, Flt3L, CCL3, CCL5, GM-CSF, etc.); an agonist of a co-stimulatory molecule (e.g., an agonist of ICOS, ICOS-L, OX40, OX40L, etc.); or a cancer antigen (e.g., a tumor associated antigen). See, e.g., Section 5.7.2 for examples of checkpoint inhibitors and agonists of co-stimulatory molecules. In some embodiments, the recombinant oncolytic virus is pexastimogene devacirepvec (Pexa-Vec, formerly named JX-594), ONCOS (adeno Δ24-RGD-GM-CSF insertion), herpes virus OrienX010, ICOVIR-5, Talimogene Laherparepvec (T-VEC, Imlygic®), VV JX-594, Ad Ad5/3-D24-GMCSF, or CG0070.

In certain embodiments in which a recombinant oncolytic viruses comprises a genome, wherein the genome comprises a first transgene comprising a nucleotide sequence encoding a VEGF-C agent, a VEGF-D agent, or both, or a first transgene comprising a nucleotide sequence encoding a VEGF-C agent and a second transgene comprising a nucleotide sequence encoding a VEGF-D agent, the genome does not further comprise a transgene comprising a nucleotide sequence encoding a heterologous protein. In some embodiments in which a recombinant oncolytic viruses comprises a genome, wherein the genome comprises a first transgene comprising a nucleotide sequence encoding a VEGF-C agent, a VEGF-D agent, or both, or a first transgene comprising a nucleotide sequence encoding a VEGF-C agent and a second transgene comprising a nucleotide sequence encoding a VEGF-D agent, the genome does not further comprise a transgene comprising a nucleotide sequence encoding a heterologous antigen (e.g., a bacterial antigen, a viral antigen, a fungal antigen, a protozoal antigen, a helminth antigen or a cancer antigen). In certain embodiments in which a recombinant oncolytic viruses comprises a genome, wherein the genome comprises a first transgene comprising a nucleotide sequence encoding a VEGF-C agent, a VEGF-D agent, or both, or a first transgene comprising a nucleotide sequence encoding a VEGF-C agent and a second transgene comprising a nucleotide sequence encoding a VEGF-D agent, the genome does not further comprise a transgene comprising a nucleotide sequence encoding a checkpoint inhibitor (e.g., an antibody that specifically binds to PD-1 and blocks binding of PD-1 to PDL1, PDL2 or both, such as pembrolizumab or nivolumab; an antibody that specifically binds to PDL1 and blocks binding of PDL1 to PD1, CD80 or both, such as atezolizumab, durvalumab, or cemiplimab; an antibody that specifically binds to CLTA-4 and block the interaction of CTLA-4 with its ligands B7.1 and B7.2, such as ipilimumab or tremelimumab; and an antibody that specifically binds to TIM3); a cytokine (e.g., IL-2, IL-12, IL-15, IFN alpha/beta, 41-BB, CD40L, Flt3L, CCL3, CCL5, GM-CSF, etc.); an agonist of a co-stimulatory molecule (e.g., an agonist of ICOS, ICOS-L, OX40, OX40L, etc.); or a cancer antigen (e.g., tumor associated antigen). See, e.g., Section 5.7.2 for examples of checkpoint inhibitors and co-stimulatory molecules. In some embodiments, the recombinant oncolytic virus is pexastimogene devacirepvec (Pexa-Vec, formerly named JX-594), ONCOS (adeno Δ24-RGD-GM-CSF insertion), herpes virus OrienX010, ICOVIR-5, Talimogene Laherparepvec (T-VEC, Imlygic®), VV JX-594, Ad Ad5/3-D24-GMCSF, or CG0070.

In certain embodiments, a recombinant oncolytic viruses comprises a genome, wherein the genome comprises a transgene comprising a nucleotide sequence encoding a VEGF-C agent, a VEGF-D agent, or both, operably linked to a regulatory sequence (e.g., a promoter, enhancer or both).

### 5.3.1 Recombinant APMV

In one aspect, provided herein are recombinant APMVs comprising a genome, wherein the genome comprises a transgene comprising a nucleotide sequence encoding a VEGFR-3-activating agent. In one embodiment, a VEGFR-3-activating agent is a VEGF-C protein or a VEGF-D protein. In another embodiment, a VEGFR-3-activating agent is a nucleic acid sequence comprising a nucleotide sequence encoding a VEGF-C protein or a VEGF-D protein. The VEGF-C protein or VEGF-D protein may be a derivative of a naturally occurring form of VEGF-C or VEGF-D, respectively. See Section 5.2 and 5.3.2 for examples of VEGF-C proteins, VEGF-D proteins, nucleic acid sequences encoding a VEGF-C protein, and nucleic acid sequences encoding a VEGF-D protein, VEGF-C derivatives, and VEGF-D derivatives.

In another aspect, presented herein are recombinant APMVs comprising a packaged genome, wherein the packaged genome comprises a transgene comprising a nucleotide sequence encoding a VEGF-C agent, a VEGF-D agent, or both. In another aspect, provided herein are recombinant APMVs comprising a packaged genome, wherein the genome comprises a transgene comprising a nucleotide sequence encoding a VEGF-C agent and a transgene comprising a nucleotide sequence encoding a VEGF-D agent. See, e.g., Section 5.1.1, 5.3.1.1 and Section 6 for examples of APMVs, the genome of which a transgene encoding a VEGF-C agent or a VEGF-D agent may be incorporated. In a particular embodiment, the genome of the APMV, which the transgene comprising a nucleotide sequence encoding a VEGF-C agent or a VEGF-D agent is incorporated, is the genome of an APMV-1 (e.g., an APMV-1 strain described herein), APMV-2 (e.g., an APMV-2 strain described herein), APMV-3 (e.g., an APMV-3 strain described herein), APMV-4 (e.g., an APMV-4 strain described herein), APMV-5, (e.g., an APMV-4 strain described herein), APMV-6 (e.g., an APMV-4 strain described herein), APMV-7 strain (e.g., an APMV-7 strain described herein), APMV-8 strain (e.g., an APMV-8 strain described herein), or APMV-9 (e.g., an APMV-4 strain described herein). In another embodiment, the genome of the APMV in which the transgene comprising a nucleotide sequence encoding a VEGF-C agent or a VEGF-D agent is incorporated is the genome of an APMV-6 (e.g., an APMV-6 strain described herein) or APMV-9 strain (e.g., an APMV-9 strain described herein). In a specific embodiment, provided herein is a recombinant APMV-1 comprising a packaged genome, wherein the packaged genome comprises a transgene comprising a nucleotide sequence encoding a VEGF-C agent or a VEGF-D agent. In a preferred embodiment, provided herein is a recombinant APMV-4 comprising a packaged genome, wherein the packaged genome comprises (consists of) the negative sense RNA transcribed from the cDNA sequence set forth in SEQ ID NO: 88. In a specific embodiment, provided herein is a recombinant APMV-4 comprising a packaged genome, wherein the packaged genome comprises a transgene comprising a nucleotide sequence encoding a VEGF-C agent or a VEGF-D agent. In a preferred embodiment, provided herein is a recombinant APMV-4 comprising a packaged genome, wherein the packaged genome comprises (consists of) the negative sense RNA transcribed from the cDNA sequence set forth in SEQ ID NO: 90. In a specific embodiment, the protein encoded by the transgene is expressed by cells infected with the recombinant APMV.

In certain embodiments, the genome of the recombinant APMV does not comprise a heterologous sequence encoding a heterologous protein other than the protein encoded by the transgene comprising a VEGF-C agent or a VEGF-D agent. In certain embodiments, a recombinant APMV described herein comprises a packaged genome, wherein the genome comprises (or consists of) the genes found in APMV and a transgene. In certain embodiments, a recombinant APMV described herein comprises a packaged genome, wherein the genome comprises (or consists of) the transcription units found in APMV (e.g., transcription units for APMV nucleocapsid, protein, phosphoprotein, matrix protein, fusion protein, hemagglutinin-neuraminidase protein, and large polymerase protein) and a transgene (e.g., in Section 5.3.2), but does not include another other transgenes.

5.3.1.1 Backbone of the Recombinant APMV

Any APMV-1, APMV-2, APMV-3, APMV-4, APMV-5, APMV-6, APMV-7, APMV-8, or APMV-9 strain may serve as the “backbone” that is engineered to encode a transgene described herein, including, but not limited to, naturally-occurring strains, variants or mutants, mutagenized viruses, or genetically engineered viruses, or any combination thereof. See, e.g., section 5.1 and 6 for examples of APMV that may be engineered to encode a transgene described herein. In certain embodiments, the APMV-1, APMV-2, APMV-3, APMV-4, APMV-5, APMV-6, APMV-7, APMV-8, or APMV-9 strain that is engineered to encode a transgene described herein is a lytic strain. In other embodiments, the APMV-1, APMV-2, APMV-3, APMV-4, APMV-5, APMV-6, APMV-7, APMV-8, or APMV-9 strain that is engineered to encode a transgene described herein is a non-lytic strain. In a specific embodiment, a transgene described herein is incorporated into the genome of APMV-1, APMV-2, APMV-3, APMV-4, APMV-5, APMV-6, APMV-7, APMV-8, or APMV-9 strain that is avirulent in an avian(s) by a method(s) described herein or known to one of skill in the art. In certain embodiments, the APMV-APMV-2, APMV-3, APMV-4, APMV-5, APMV-6, APMV-7, APMV-8, or APMV-9 strain that is engineered to encode a transgene described herein is genetically engineered to be attenuated in a manner that attenuates the pathogenicity of the virus in birds.

In another specific embodiment, a transgene is incorporated into the genome of an APMV-1, APMV-2, APMV-3, APMV-4, APMV-5, APMV-6, APMV-7, APMV-8, or APMV-9 strain that has an intracerebral pathogenicity index in day-old chicks of the Gallus gallus species of less than 0.7. In certain specific embodiments, the APMV-1, APMV-2, APMV-3, APMV-4, APMV-5, APMV-6, APMV-7, APMV-8, or APMV-9 strain that is engineered to encode a transgene described herein is not pathogenic as assessed by intracranial injection of 1-day-old chicks with the virus, and disease development and death as scored for 8 days. In some embodiments, the APMV-1, APMV-2, APMV-3, APMV-4, APMV-5, APMV-6, APMV-7, APMV-8, or APMV-9 strain that is engineered to encode a transgene described herein has an intracranial pathogenicity index of less than 0.7, less than 0.6, less than 0.5, less than 0.4, less than 0.3, less than 0.2 or less than 0.1. In some embodiments, the APMV-1, APMV-2, APMV-3, APMV-4, APMV-5, APMV-6, APMV-7, APMV-8, or APMV-9 strain that is engineered to encode a transgene described herein has an intracranial pathogenicity index between 0.7 to 0.1, 0.6 to 0.1, 0.5 to 0.1 or 0.4 to 0.1. In certain embodiments, the APMV-1, APMV-2, APMV-3, APMV-4, APMV-5, APMV-6, APMV-7, APMV-8, or APMV-9 strain that is engineered to encode a transgene described herein has an intracranial pathogenicity index of zero. See, e.g., one or more of the following references for a description of an assay that may be used to assess the pathogenicity of an APMV in birds: Hines, N. L. and C. L. Miller, Avian paramyxovirus serotype-1: a review of disease distribution, clinical symptoms, and laboratory diagnostics. Vet Med Int, 2012. 2012: p. 708216; Kim S-H, Xiao S, Shive H, Collins P L, Samal S K., 2012: Replication, Neurotropism, and Pathogenicity of Avian Paramyxovirus Serotypes 1-9 in Chickens and Ducks. PLoS ONE. 7(4): e34927; Subbiah, M., Xiao, S., Khattar, S. K., Dias, F. M., Collins, P. L., & Samal, S. K., 2010: Pathogenesis of two strains of Avian Paramyxovirus serotype 2, Yucaipa and Bangor, in chickens and turkeys. Avian Diseases, 54(3), 1050-1057; Kumar S, Militino Dias F, Nayak B, Collins P L, Samal S. K., 2010: Experimental avian paramyxovirus serotype-3 infection in chickens and turkeys. Veterinary Research.; 41(5):72; Ryota Tsunekuni, Hirokazu Hikono, Takehiko Saito, 2014: Evaluation of avian paramyxovirus serotypes 2 to 10 as vaccine vectors in chickens previously immunized against Newcastle disease virus. Veterinary Immunology and Immunopathology; 160(3-4):184-191; and www.oie.int/fileadmin/Home/fr/Health_standards/tahm/2.03.14 NEWCASTLE DIS.pdf, each of which is incorporated herein by reference in its entirety.

In a specific embodiment, a transgene described herein is incorporated into the genome of an APMV-1, APMV-2, APMV-3, APMV-4, APMV-5, APMV-6, APMV-7, APMV-8, or APMV-9 that decreases tumor growth and increases survival in a B16-F10/VEGF-C+ syngeneic murine melanoma model as compared to tumor growth and survival in a B16-F10/VEGF-C+ syngeneic murine melanoma model administered phosphate buffered saline (PBS).

In a specific embodiment, a transgene described herein is incorporated into the genome of an APMV-1 strain. In a specific embodiment, the APMV-1 strain is a naturally-occurring strain. In certain embodiments, the APMV-1 is a lytic strain. In other embodiments, the APMV-APMV-1 strain is a non-lytic strain. In certain embodiments, the APMV-1 strain is lentogenic strain. In some embodiments, the APMV-1 strain is a mesogenic strain. In other embodiments, the APMV-1 strain is a velogenic strain. See, e.g., Newcastle Disease, Avian Paramyoxvirus-1 Infection, Goose Paramyoxvirus Infection, Ranikhet disease, the Center for Food Security & Public Health, Iowa State University, Institute for International Cooperation in Animal Biologies, College of Veterinary Medicine, Iowa State University, pp. 1-9 (January 2016) for a discussion regarding lentogenic, mesogenic and velogenic APMV-1 (otherwise referred to as NDV) strains, which is incorporated herein by reference in its entirety. Specific examples of APMV-1 strains include, but are not limited to, the 73-T strain, NDV HUJ strain, Ulster strain (see, e.g., GenBank No. U25837), MTH-68 strain, Italien strain (see, e.g., GenBank No. EU293914), Hickman strain (see, e.g., Genbank No. AF309418), PV701 strain, Hitchner B1 strain (see, e.g., GenBank No. AF309418 or NC 002617), La Sota strain (see, e.g., GenBank Nos. AY845400 and JF950510.1 and GI No. 56799463), YG97 strain (see, e.g., GenBank Nos. AY351959 or AY390310), MET95 strain (see, e.g., GenBank No. AY143159), Roakin strain (see, e.g., GenBank No. AF124443), and F48E9 strain (see, e.g., GenBank Nos. AF163440 and U25837). In a specific embodiment, the APMV-1 strain is the Hitchner B1 strain. In another specific embodiment, the APMV-1 strain is a B1 strain as identified by GenBank No. AF309418 or NC 002617. In another specific embodiment, the APMV-1 strain is the NDV identified by ATCC No. VR2239. In another specific embodiment, the APMV-1 strain is an NDV described in U.S. Pat. No. 10,035,984, which is incorporated herein by reference in its entirety.

In a specific embodiments, a transgene described herein is incorporated into the genome of an APMV-1 that is genetically modified. In one embodiment, a transgene described herein is incorporated into the genome of an APMV-1 strain that is engineered to express a mutated F protein with a mutated cleavage site. In a specific embodiment, a transgene described herein is incorporated into the genome of an APMV-1 strain that is engineered to express a mutated F protein in which the cleavage site of the F protein is mutated to produce a polybasic amino acid sequence, which allows the protein to be cleaved by intracellular proteases, which makes the virus more effective in entering cells and forming syncytia. In another specific embodiment, a transgene described herein is incorporated into the genome of an APMV-1 strain that is engineered to express a mutated F protein in which the cleavage site of the F protein is replaced with a mutated cleavage site containing one or two extra arginine residues, allowing the mutant cleavage site to be activated by ubiquitously expressed proteases of the furin family. Specific examples of NDVs that express such a mutated F protein include, but are not limited to, rNDV/F2aa and rNDV/F3aa. For a description of mutations introduced into a NDV F protein to produce a mutated F protein with a mutated cleavage site, see, e.g., Park et al. (2006) Engineered viral vaccine constructs with dual specificity: avian influenza and Newcastle disease. PNAS USA 103: 8203-2808, which is incorporated herein by reference in its entirety.

In another embodiment, a transgene described herein is incorporated into the genome of an APMV-1 strain that is naturally occurring. In a specific embodiment, an APMV-1 strain that is naturally occurring and has an intracerebral pathogenicity index in day-old chicks of the Gallus gallus species of less than 0.7 is used in a method of treating cancer described herein. In a specific embodiment, an APMV-1 that is used in a method of treating cancer described herein is an APMV-1 with a genome that has 80%, 85%, 90%, 95% or higher percent identity to the genome of a LaSota strain (e.g., SEQ ID NO: 83 or 84).

In a specific embodiment, a transgene described herein is incorporated into the genome of an APMV-1 strain that is engineered to express a mutated F protein with the amino acid mutation L289A (i.e., an L to A mutation at the amino acid position corresponding to L289 of the LaSota F protein). For a description of the L289A mutation, see, e.g., Sergei et al. (2000) A Single Amino Acid Change in the Newcastle Disease Virus Fusion Protein Alters the Requirement for UN Protein in Fusion. Journal of Virology 74(11): 5101-5107, which is incorporated herein by reference in its entirety. In specific embodiments, the L289A mutated F protein possesses one, two or three arginine residues in the cleavage site. In a specific embodiment, a transgene described herein is incorporated into the genome of a LaSota strain, which has been engineered to express a mutated F protein with the amino acid mutation L289A (i.e., an L to A mutation at the amino acid position corresponding to L289 of the LaSota F protein). In a specific embodiment, the genetically modified NDV LaSota strain comprises a packaged genome, wherein the packaged genome comprises the negative sense RNA transcribed from the cDNA sequence set forth in SEQ ID NO: 83 or 84.

In some embodiments, a transgene described herein is incorporated into the genome of an APMV-1 strain disclosed in Kim et al., 2017, PLOS ONE 12(3): e0173965 and Kim et al., 2016, J. of General Virology 97: 1297-1303, each of which is incorporated herein by reference in its entirety.

In certain embodiments, a transgene described herein is incorporated into the genome of an APMV-1 strain that comprises a nucleotide sequence encoding a mutated F protein with an F protein cleavage site of NDV LaSota strain or glycoprotein B of cytomegalovirus (CMV). In a specific embodiment, a transgene described herein is incorporated into the genome of an APMV-1 strain that comprises a nucleotide sequence encoding a mutated F protein with an F protein cleavage having one of the following sequence modifications: S116: ¹¹¹H-N-R-T-K-S/F¹¹⁷ (SEQ ID NO: 91); S116K: ¹¹¹H-K-T-K-S/F¹¹⁷ (SEQ ID NO: 92); S116M: ¹¹¹H-N-R-M-K-S/F¹¹⁷ (SEQ ID NO: 93); S116KM: ¹¹¹H-N-K-M-K-S/F-I¹¹⁸ (SEQ ID NO: 94); or R116: ¹¹¹H-N-R-T-K-R/F-I¹¹⁸ (SEQ ID NO: 95), such as described in International Patent Application No. WO 2015/032755. See, e.g., International Patent Application Publication No. WO 2015/032755 for a description of the types of mutated F protein cleavage sites that may be engineered into an NDV F protein, which is incorporated herein by reference in its entirety. In some embodiments, the mutated F protein is in addition to the backbone NDV F protein. In specific embodiments, the mutated F protein replaces the backbone NDV F protein.

In a specific embodiment, a transgene described herein is incorporated into the genome of an APMV-4 strain. In a preferred embodiment, a transgene described herein is incorporated into the genome of APMV-4/Duck/Hong Kong/D3/1975 strain. One example of a cDNA sequence of the genome of the APMV-4/Duck/Hong Kong/D3/1975 strain may be found in SEQ ID NO:78. In a specific embodiment, the nucleotide sequence of a transgene described herein is incorporated into the genome of APMV-4/Duck/China/G302/2012 strain, APMV4/mallard/Belgium/15129/07 strain, APMV4/Uriah_aalge/Russia/Tyuleniy_Island/115/2015 strain, APMV4/Egyptian goose/South Africa/N1468/2010 strain, or APMV-4/duck/Delaware/549227/2010 strain. One example of a cDNA sequence of the genome of the APMV-4/Duck/China/G302/2012 strain may be found in SEQ ID NO:81. An example of a cDNA sequence of the genome of the APMV4/mallard/Belgium/15129/07 strain may be found in SEQ ID NO:77. An example of a cDNA sequence of the genome of the APMV4/Uriah_aalge/Russia/Tyuleniy_Island/115/2015 strain may be found in SEQ ID NO:79. An example of a cDNA sequence of the genome of the APMV4/Egyptian goose/South Africa/N1468/2010 strain may be found in SEQ ID NO:80. An example of a cDNA sequence of the genome of the APMV-4/duck/Delaware/549227/2010 strain may be found in SEQ ID NO:82. In another specific embodiment, an APMV-4 comprises the cDNA sequence provided in SEQ ID NO: 86. In another specific embodiment, an APMV-4 comprises a cDNA sequence provided in Table 3 or Section 6, infra.

In a specific embodiment, a transgene described herein is incorporated into the genome of an APMV-4 that decreases tumor growth and increases survival in a B16-F10/VEGF-C+ syngeneic murine melanoma model as compared to tumor growth and survival in a B16-F10/VEGF-C+ syngeneic murine melanoma model administered phosphate buffered saline (PBS).

In a specific embodiment, a transgene described herein is incorporated into the genome of an APMV-7 strain. In a particular embodiment, a transgene described herein is incorporated into the genome of is APMV-7/dove/Tennessee/4/75. See, e.g., GenBank No. FJ231524.1 for the complete genomic cDNA of APMV-7/dove/Tennessee/4/75.

In a specific embodiment, a transgene described herein is incorporated into the genome of an APMV-8 strain. In a particular embodiment, a transgene described herein is incorporated into the genome of APMV-8/Goose/Delaware/1053/76. See, e.g., GenBank No. FJ619036.1 for the complete genomic cDNA sequence of APMV-8/Goose/Delaware/1053/76.

In a specific embodiment, a transgene described herein is incorporated into the genome of an APMV-9 strain. In a particular embodiment, a transgene described herein is incorporated into the genome of APMV-9 duck/New York/22/1978. See, e.g., GenBank No. NC 025390.1 for the complete genomic cDNA sequence of APMV-9 duck/New York/22/1978.

In a specific embodiment, a transgene described herein is incorporated into the genome of an APMV-2 strain. In a particular embodiment, a transgene described herein is incorporated into the genome of APMV-2 Chicken/California/Yucaipa/1956. See, e.g., GenBank No. EU338414.1 for the complete genomic cDNA sequence of APMV-2 Chicken/California/Yucaipa/1956.

In a specific embodiment, a transgene described herein is incorporated into the genome of an APMV-3 strain. In a particular embodiment, a transgene described herein is incorporated into the genome of APMV-3 turkey/Wisconsin/68. See, e.g., GenBank No. EU782025.1 for the complete genomic cDNA sequence of APMV-3 turkey/Wisconsin/68.

In a specific embodiment, a transgene described herein is incorporated into the genome of an APMV-6 strain. In a particular embodiment, a transgene described herein is incorporated into the genome of APMV-6/duck/Hong Kong/18/199/77. See, e.g., GenBank No. EU622637.2 for the complete genomic cDNA sequence of APMV-6/duck/Hong Kong/18/199/77.

One skilled in the art will understand that the APMV genomic RNA sequence is the reverse complement of a cDNA sequence encoding the APMV genome. Thus, any program that generates converts a nucleotide sequence to its reverse complement sequence may be utilized to convert a cDNA sequence encoding an APMV genome into the genomic RNA sequence (see, e.g., www.bioinformatics.org/sms/rev_comp.html, www.fr33.net/seqedit.php, and DNAStar). Accordingly, the nucleotide sequences provided in Tables 2 and 3, infra, may be readily converted to the negative-sense RNA sequence of the APMV genome by one of skill in the art.

In specific embodiments, a transgene described herein is incorporated into the genome of an APMV-1, APMV-2, APMV-3, APMV-4, APMV-5, APMV-6, APMV-7, APMV-8, or APMV-9 strain, wherein the genome comprises the transcription units of the APMV-4 strain necessary for infection and replication of the virus in a substrate (e.g., a cell line susceptible to APMV-4 infection), subject (e.g., a human subject), or both. In a specific embodiment, a transgene described herein is incorporated into the genome of an APMV-1, APMV-2, APMV-3, APMV-4, APMV-5, APMV-6, APMV-7, APMV-8, or APMV-9 strain, wherein the genome comprises a transcription unit encoding the APMV-4 nucleocapsid (N) protein, a transcription unit encoding the APMV phosphoprotein (P), a transcription unit encoding the APMV matrix (M) protein, a transcription unit encoding the APMV fusion (F) protein, a transcription unit encoding the APMV hemagglutinin-neuraminidase (HN) protein, and a transcription unit encoding the APMV large polymerase (L) protein. The transgene may be incorporated into the APMV genome between two transcription units of an APMV described herein (e.g., between the M and P transcription units or between the HN and L transcription units). In certain embodiments, the genome of the APMV does not encode a heterologous protein other than a transgene described herein.

In a specific embodiment, a transgene described herein is incorporated into the genome of an APMV-1 strain, wherein the genome comprises the transcription units of the APMV-1 strain necessary for infection and replication of the virus in a substrate (e.g., a cell line susceptible to APMV-1 infection), subject (e.g., a human subject), or both. In a specific embodiment, a transgene is incorporated into the genome of an APMV-1 strain, wherein the genome comprises a transcription unit encoding the APMV-1 nucleocapsid (N) protein, a transcription unit encoding the APMV-4 phosphoprotein (P), a transcription unit encoding the APMV-1 matrix (M) protein, a transcription unit encoding the APMV-1 fusion (F) protein, a transcription unit encoding the APMV-1 hemagglutinin-neuraminidase (HN) protein, and a transcription unit encoding the APMV-1 large polymerase (L) protein. The transgene may be incorporated into the APMV-4 genome between two transcription units of an APMV-1 described herein (e.g., between the M and P transcription units or between the HN and L transcription units). In certain embodiments, the genome of the APMV-1 does not encode a heterologous protein other than a transgene described herein. In a specific embodiment, the APMV-1 strain is an APMV-1 described herein (e.g., in this section, Section 5.1.1 or Section 6), such as a LaSota strain or a LaSota strain comprising a mutated F protein.

In a specific embodiment, a transgene described herein is incorporated into the genome of an APMV-4 strain, wherein the genome comprises the transcription units of the APMV-4 strain necessary for infection and replication of the virus in a substrate (e.g., a cell line susceptible to APMV-4 infection), subject (e.g., a human subject), or both. In a specific embodiment, a transgene described herein is incorporated into the genome of an APMV-4 strain, wherein the genome comprises a transcription unit encoding the APMV-4 nucleocapsid (N) protein, a transcription unit encoding the APMV-4 phosphoprotein (P), a transcription unit encoding the APMV-4 matrix (M) protein, a transcription unit encoding the APMV-4 fusion (F) protein, a transcription unit encoding the APMV-4 hemagglutinin-neuraminidase (HN) protein, and a transcription unit encoding the APMV-4 large polymerase (L) protein. The transgene may be incorporated into the APMV-4 genome between two transcription units of an APMV-4 described herein (e.g., between the M and P transcription units or between the HN and L transcription units). In certain embodiments, the genome of the APMV-4 does not encode a heterologous protein other than a transgene described herein. In a specific embodiment, the APMV-4 strain is the APMV-4/Duck/Hong Kong/D3/1975 strain, APMV-4/Duck/China/G302/2012 strain, APMV4/mallard/Belgium/15129/07 strain, APMV4Uriah-aalge/Russia/Tyuleniy_Island/115/2015 strain, APMV4/Egyptian goose/South Africa/NJ468/2010 strain, or APMV4/duck/Delaware/549227/2010 strain.

In a specific embodiment, a transgene described herein is incorporated into the genome of an APMV-8 strain, wherein the genome comprises the transcription units of the APMV-8 strain necessary for infection and replication of the virus in a substrate (e.g., a cell line susceptible to APMV-8 infection), subject (e.g., a human subject), or both. In a specific embodiment, a transgene described herein is incorporated into the genome of an APMV-8 strain, wherein the genome comprises a transcription unit encoding the APMV-8 nucleocapsid (N) protein, a transcription unit encoding the APMV-8 phosphoprotein (P), a transcription unit encoding the APMV-8 matrix (M) protein, a transcription unit encoding the APMV-8 fusion (F) protein, a transcription unit encoding the APMV-8 hemagglutinin-neuraminidase (HN) protein, and a transcription unit encoding the APMV-8 large polymerase (L) protein. The transgene may be incorporated into the APMV-8 genome between two transcription units of an APMV-8 described herein (e.g., between the M and P transcription units or between the HN and L transcription units). In certain embodiments, the genome of the APMV-8 does not encode a heterologous protein other than a transgene described herein. In a specific embodiment, the APMV-8 strain is the APMV-8/Goose/Delaware/1053/76 strain.

In a specific embodiment, a transgene described herein is incorporated into the genome of an APMV-9 strain, wherein the genome comprises the transcription units of the APMV-9 strain necessary for infection and replication of the virus in a substrate (e.g., a cell line susceptible to APMV-9 infection), subject (e.g., a human subject), or both. In a specific embodiment, a transgene described herein is incorporated into the genome of an APMV-9 strain, wherein the genome comprises a transcription unit encoding the APMV-9 nucleocapsid (N) protein, a transcription unit encoding the APMV-9 phosphoprotein (P), a transcription unit encoding the APMV-9 matrix (M) protein, a transcription unit encoding the APMV-9 fusion (F) protein, a transcription unit encoding the APMV-9 hemagglutinin-neuraminidase (HN) protein, and a transcription unit encoding the APMV-9 large polymerase (L) protein. The transgene may be incorporated into the APMV-9 genome between two transcription units of an APMV-9 described herein (e.g., between the M and P transcription units or between the HN and L transcription units). In certain embodiments, the genome of the APMV-9 does not encode a heterologous protein other than a transgene described herein. In a specific embodiment, the APMV-9 strain is the APMV-9 duck/New York/22/1978 strain.

In a specific embodiment, a transgene described herein is incorporated into the genome of an APMV-7 strain, wherein the genome comprises the transcription units of the APMV-7 strain necessary for infection and replication of the virus in a substrate (e.g., a cell line susceptible to APMV-7 infection), subject (e.g., a human subject), or both. In a specific embodiment, a transgene described herein is incorporated into the genome of an APMV-7 strain, wherein the genome comprises a transcription unit encoding the APMV-7 nucleocapsid (N) protein, a transcription unit encoding the APMV-7 phosphoprotein (P), a transcription unit encoding the APMV-7 matrix (M) protein, a transcription unit encoding the APMV-7 fusion (F) protein, a transcription unit encoding the APMV-7 hemagglutinin-neuraminidase (HN) protein, and a transcription unit encoding the APMV-7 large polymerase (L) protein. The transgene may be incorporated into the APMV-7 genome between two transcription units of an APMV-7 described herein (e.g., between the M and P transcription units or between the HN and L transcription units). In certain embodiments, the genome of the APMV-7 does not encode a heterologous protein other than a transgene described herein. In a specific embodiment, the APMV-7 strain is the APMV-7/dove/Tennessee/4/75 strain.

In a specific embodiment, a transgene described herein is incorporated into the genome of an APMV-2 strain, wherein the genome comprises the transcription units of the APMV-2 strain necessary for infection and replication of the virus in a substrate (e.g., a cell line susceptible to APMV-2 infection), subject (e.g., a human subject), or both. In a specific embodiment, a transgene described herein is incorporated into the genome of an APMV-2 strain, wherein the genome comprises a transcription unit encoding the APMV-2 nucleocapsid (N) protein, a transcription unit encoding the APMV-2 phosphoprotein (P), a transcription unit encoding the APMV-2 matrix (M) protein, a transcription unit encoding the APMV-2 fusion (F) protein, a transcription unit encoding the APMV-2 hemagglutinin-neuraminidase (HN) protein, and a transcription unit encoding the APMV-2 large polymerase (L) protein. The transgene may be incorporated into the APMV-2 genome between two transcription units of an APMV-2 described herein (e.g., between the M and P transcription units or between the HN and L transcription units). In certain embodiments, the genome of the APMV-2 does not encode a heterologous protein other than a transgene described herein. In a specific embodiment, the APMV-2 strain is the APMV-2 Chicken/California/Yucaipa/1956 strain.

In a specific embodiment, a transgene described herein is incorporated into the genome of an APMV-3 strain, wherein the genome comprises the transcription units of the APMV-3 strain necessary for infection and replication of the virus in a substrate (e.g., a cell line susceptible to APMV-3 infection), subject (e.g., a human subject), or both. In a specific embodiment, a transgene described herein is incorporated into the genome of an APMV-3 strain, wherein the genome comprises a transcription unit encoding the APMV-3 nucleocapsid (N) protein, a transcription unit encoding the APMV-3 phosphoprotein (P), a transcription unit encoding the APMV-3 matrix (M) protein, a transcription unit encoding the APMV-3 fusion (F) protein, a transcription unit encoding the APMV-3 hemagglutinin-neuraminidase (HN) protein, and a transcription unit encoding the APMV-3 large polymerase (L) protein. The transgene may be incorporated into the APMV-3 genome between two transcription units of an APMV-3 described herein (e.g., between the M and P transcription units or between the HN and L transcription units). In certain embodiments, the genome of the APMV-3 does not encode a heterologous protein other than a transgene described herein. In a specific embodiment, the APMV-3 strain is the APMV-3 turkey/Wisconsin/68 strain.

In a specific embodiment, a transgene described herein is incorporated into the genome of an APMV-6 strain, wherein the genome comprises the transcription units of the APMV-6 strain necessary for infection and replication of the virus in a substrate (e.g., a cell line susceptible to APMV-6 infection), subject (e.g., a human subject), or both. In a specific embodiment, a transgene described herein is incorporated into the genome of an APMV-6 strain, wherein the genome comprises a transcription unit encoding the APMV-6 nucleocapsid (N) protein, a transcription unit encoding the APMV-6 phosphoprotein (P), a transcription unit encoding the APMV-6 matrix (M) protein, a transcription unit encoding the APMV-6 fusion (F) protein, a transcription unit encoding the APMV-6 hemagglutinin-neuraminidase (HN) protein, and a transcription unit encoding the APMV-6 large polymerase (L) protein. The transgene may be incorporated into the APMV-6 genome between two transcription units of an APMV-6 described herein (e.g., between the M and P transcription units or between the HN and L transcription units). In certain embodiments, the genome of the APMV-6 does not encode a heterologous protein other than a transgene described herein. In a specific embodiment, the APMV-6 strain is the APMV-6/duck/Hong Kong/18/199/77 strain.

In certain embodiments in which a recombinant APMV comprising a packaged genome, wherein the genome comprises a first transgene comprising a nucleotide sequence encoding a VEGF-C agent, a VEGF-D agent, or both, or a first transgene comprising a nucleotide sequence encoding a VEGF-C agent and a second transgene comprising a nucleotide sequence encoding a VEGF-D agent, the genome does not further comprise a transgene comprising a nucleotide sequence encoding a heterologous protein. In some embodiments in which a recombinant APMV comprising a packaged genome, wherein the genome comprises a first transgene comprising a nucleotide sequence encoding a VEGF-C agent, a VEGF-D agent, or both, or a first transgene comprising a nucleotide sequence encoding a VEGF-C agent and a second transgene comprising a nucleotide sequence encoding a VEGF-D agent, the genome does not further comprise a transgene comprising a nucleotide sequence encoding a heterologous antigen (e.g., a bacterial antigen, a viral antigen, a fungal antigen, a protozoal antigen, a helminth antigen or a cancer antigen). In certain embodiments in which a recombinant APMV comprising a packaged genome, wherein the genome comprises a first transgene comprising a nucleotide sequence encoding a VEGF-C agent, a VEGF-D agent, or both, or a first transgene comprising a nucleotide sequence encoding a VEGF-C agent and a second transgene comprising a nucleotide sequence encoding a VEGF-D agent, the genome does not further comprise a transgene comprising a nucleotide sequence encoding a checkpoint inhibitor (e.g., an antibody that specifically binds to PD-1 and blocks binding of PD-1 to PDL1, PDL2 or both, such as pembrolizumab or nivolumab; an antibody that specifically binds to PDL1 and blocks binding of PDL1 to PD1, CD80 or both, such as atezolizumab, durvalumab, or cemiplimab; and an antibody that specifically binds to CLTA-4 and block the interaction of CTLA-4 with its ligands B7.1 and B7.2, such as ipilimumab or tremelimumab and an antibody that specifically binds to TIM3); a cytokine (e.g., IL-2, IL-12, IL-15, IFN alpha/beta, 41-BB, CD40L, Flt3L, CCL3, CCL5, GM-CSF, etc.); an agonist of a co-stimulatory molecule (e.g., an agonist of ICOS, ICOS-L, OX40, OX40L, etc.); a cancer antigen (e.g., tumor associated antigen), or a heterologous antigen (e.g., a bacterial, viral, fungal, protozoal, or helminth antigen). See, e.g., Section 5.7.2 for examples of checkpoint inhibitors and co-stimulatory molecules.

In a specific embodiment, a recombinant APMV is one described in Section 6, infra. In certain embodiments, a recombinant APMV has the characteristics of a recombinant APMV as described in Section 6, infra. In some embodiments, a recombinant APMV results in one, two or more effects in a tumor, lymph node, or both when administered to a subject with cancer as described in Section 6, infra.

### 5.3.2 Transgenes

In one aspect, provided herein is a transgene comprising a nucleotide sequence encoding a VEGFR-3-activating agent. In one embodiment, a VEGFR-3-activating agent is a VEGF-C protein or a VEGF-D protein. In another embodiment, a VEGFR-3-activating agent is a nucleic acid sequence comprising a nucleotide sequence encoding a VEGF-C protein or a VEGF-D protein. The VEGF-C protein or VEGF-D protein may be derivatives of VEGF-C or VEGF-D, respectively. See Sections 5.2, 5.3.2 and 6 for examples of VEGF-C proteins, VEGF-D proteins, nucleic acid sequences encoding a VEGF-C protein, nucleic acid sequences encoding a VEGF-D protein, VEGF-C derivatives, and VEGF-D derivatives. See Table 3 for exemplary VEGF-C and VEGF-D nucleotide and amino acid sequences.

In another aspect, provided herein is a transgene comprising a nucleotide sequence encoding a vascular endothelial growth factor-C (VEGF-C) agent. See Section 5.2, supra, for VEGF-C agents. In a specific embodiment, a transgene comprising a nucleotide sequence encoding a vascular endothelial growth factor-C (VEGF-C) agent is incorporated into the genome of an oncolytic virus described herein (e.g., APMV described herein, such as an APMV-1 or an APMV-4 described herein). The transgene may encode VEGF-C such as set forth in any one of SEQ ID NOs: 19-24, 41-46, 51 or 52. In a specific embodiment, the transgene encodes human VEGF-C such as set forth in any one of SEQ ID NOs: 41-46. See e.g., Section 5.1 and Section 5.3 supra for oncolytic viruses that may be used; with respect to types and strains of APMV that may be used, see Sections 5.1.1 and 5.3.1.1 and with respect to VEGF-C agents that may be used, see, e.g., section 5.2.

In another aspect, provided herein is a transgene comprising a nucleotide sequence encoding a vascular endothelial growth factor D (VEGF-D) agent. See Section 5.2, supra, for VEGF-D agents. In another specific embodiment, a transgene comprising a nucleotide sequence encoding a vascular endothelial growth factor-D (VEGF-D) agent is incorporated into the genome of an oncolytic virus described herein (e.g., APMV described herein, such as an APMV-1 or an APMV-4 described herein). The transgene may encode VEGF-D, such as set forth in any one of SEQ ID NO: 99-104 See, e.g., Section 5.1 and Section 5.3, supra, for oncolytic viruses that may be used; with respect to types and strains of APMV that may be used, see Sections 5.1.1 and 5.3.1.1 and with respect to VEGF-D agents that may be used, see, e.g., section 5.2.

In specific embodiments, a transgene comprising a nucleotide sequence encoding a VEGF-C agent or a VEGF-D agent comprises appropriate signals in the transgene for recognition by the virus and a valid Kozak sequence(s) (e.g., to improve eukaryotic ribosomal translation). In certain embodiments, a transgene comprising a nucleotide sequence encoding a VEGF-C agent or a VEGF-D agent comprises appropriate signals in the transgene for recognition by the virus, a valid Kozak sequence(s) (e.g., to improve eukaryotic ribosomal translation), and a restriction site to facilitate cloning. In specific embodiments, a transgene comprising a nucleotide sequence encoding a VEGF-C agent or a VEGF-D agent comprises APMV regulatory signals (e.g., gene end, intergenic, and gene start sequences) and Kozak sequences. In some embodiments, a transgene comprising a nucleotide sequence encoding a VEGF-C agent or a VEGF-D agent comprises APMV regulatory signals (e.g., gene end, intergenic, and gene start sequences), Kozak sequences and restriction sites to facilitate cloning. In certain embodiments, a transgene comprising a nucleotide sequence encoding a VEGF-C agent or a VEGF-D agent comprises APMV regulatory signals (e.g., gene end, intergenic and gene start sequences), Kozak sequences, restriction sites to facilitate cloning, and additional nucleotides in the non-coding region to ensure compliance with the rule of six. In a preferred embodiment, the transgene complies with the rule of six. In a specific embodiment, a transgene comprises the nucleotide sequence set forth in SEQ ID NO: 87 or 89.

VEGF-C

In a specific embodiment, a transgene comprising a nucleotide sequence encoding a VEGF-C agent is incorporated into the genome of an oncolytic virus described herein, such as an APMV (e.g., APMV-1 or APMV-4). See, e.g., Section 5.1. and Section 5.3, supra, for oncolytic viruses that may be used. A nucleotide sequence may encode precursor VEGF-C, pro-VEGF-C-ΔC, or mature VEGF-C. In one embodiment, a nucleotide sequence encodes a full-length form of VEGF-C. In some embodiments, a nucleotide sequence encodes unprocessed form of VEGF-C. In a specific embodiment, a nucleotide sequence encodes human VEGF-C. In another specific embodiment, human VEGF-C comprises the amino acid sequence set forth in any one of SEQ ID NOs: 41-46. In another specific embodiment, the nucleic acid sequence encoding a human VEGF-C comprises the nucleotide sequence set forth in any one of SEQ ID NOs: 29-40. In another specific embodiment, a nucleotide sequence encodes murine VEGF-C. In another specific embodiment, murine VEGF-C comprises the amino acid sequence set forth in SEQ ID NO: 19-24. In another specific embodiment, the nucleic acid sequence encoding a murine VEGF-C comprises the nucleotide sequence set forth in any one of SEQ ID NOs: 1-18. In another specific embodiment, a nucleotide sequence encodes canine VEGF-C. In another specific embodiment, canine VEGF-C comprises the amino acid sequence set forth in SEQ ID NO: 51 or 52. In another specific embodiment, the nucleic acid sequence encoding canine VEGF-C comprises the nucleotide sequence set forth in SEQ ID NO: 49 or 50. In another specific embodiment, a VEGF-C agent comprises the amino acid or nucleic acid sequence of a VEGF-C construct described in Section 6, infra.

In a specific embodiment, a transgene comprises a nucleotide sequence that encodes human VEGF-C. In certain embodiments, a nucleotide sequence encodes the amino acid sequence set forth in any one of SEQ ID NOs: 19-24, 41-46, 51, or 52. In some embodiments, the transgene comprises the nucleotide sequence set forth in any one of SEQ ID NOs: 1-18, 29-40, 49, or 50. One of skill in the art would be able to use such sequence information to produce a transgene for incorporation into the genome of an oncolytic virus described herein, such as an APMV (e.g., APMV-1 or APMV-4). For example, a transgene encoding a human VEGF-C comprising the amino acid sequence set forth in GenBank No. NM_005429.5, Uniprot P49767, or Uniprot Q6FH59 may be incorporated into the genome of any APMV type or strain described herein. In a specific embodiment, such a transgene comprises the sequence set forth in any one of SEQ ID NOs: 29-40. In some embodiments, a transgene comprises the nucleotide sequence of canine VEGF-C, such e.g., provided in GenBank™ accession numbers XM_S40047.6 and XM_02543044. However, given the degeneracy of the nucleic acid code, there are a number of different nucleic acid sequences that may encode the same VEGF-C protein. In a specific embodiment, a transgene comprising a nucleotide sequence encoding VEGF-C (e.g., human VEGF-C) is codon optimized. See, e.g., Section 5.3.2.1, infra, for a discussion regarding codon optimization. In some embodiments, the transgene comprising a nucleotide sequence encoding a human VEGF-C protein comprises the amino acid sequence encoded by the nucleic acid sequence comprising the sequence set forth in any one of SEQ ID NOs: 35-40. The transgene encoding VEGF-C (e.g., human VEGF-C) may be incorporated between any two transcription units (e.g., between the APMV P and M transcription units, or between the HN and L transcription units).

In certain embodiments, the VEGF-C may be human, dog, cat, horse, pig, or cow VEGF-C. In a specific embodiment, the VEGF-C is human VEGF-C. GenBank™ accession number NM_005429.5, Uniprot P49767, or Uniprot Q6FH59 provides an exemplary human VEGF-C nucleic acid sequence. GenBank™ accession number NM_005429.5, Uniprot P49767, or Uniprot Q6FH59 provides an exemplary human VEGF-C amino acid sequence. In some embodiments, the VEGF-C is canine VEGF-C, such e.g., provided in GenBank™ accession numbers XM_S40047.6 and XM_02543044. In specific embodiments, the VEGF-C proteins are modified by post-translational processing such as signal peptide cleavage, disulfide bond formation, glycosylation (e.g., N-linked glycosylation), protease cleavage and lipid modification (e.g., S-palmitoylation). In some embodiments, VEGF-C includes a signal sequence. In other embodiments, VEGF-C does not include a signal sequence. The signal sequence can be the naturally occurring signal peptide sequence or a variant thereof. In some embodiments, the signal peptide is a VEGF-C signal peptide. In some embodiments, the signal peptide is heterologous to a VEGF-C signal peptide. In some embodiments, the signal peptide is a Gaussia luciferase signal peptide (e.g., SEQ ID NO: 28). In certain embodiments, the signal peptide is a IgG light chain signal peptide (e.g., SEQ ID NO: 26).

In a specific embodiment, a transgene comprising a nucleotide sequence encoding a VEGF-C derivative is incorporated into the genome of an oncolytic virus described herein, such as an APMV (e.g., APMV-1 or APMV-4). See, e.g., Section 5.1 and Section 5.3.1, supra, for oncolytic viruses that may be used. In a specific embodiment, a transgene comprises a nucleotide sequence that encodes a human VEGF-C derivative. In another embodiment, a transgene comprises a nucleotide sequence that encodes a canine VEGF-C derivative. One of skill in the art would be able to use the sequence information to produce a transgene for incorporation into the genome of an comprising a nucleotide sequence. In a specific embodiment, a VEGF-C derivative has at least 70%, 75%, 80%, 85%, 90%, 95%, 96%, 98%, or 99% amino acid sequence identity to a VEGF-C known to those of skill in the art (e.g., any one of SEQ ID NOs: 19-24, 41-46, 51, or 52). In another specific embodiment, a VEGF-C derivative has at least 85%, 90%, 95%, 96%, 98%, or 99% amino acid sequence identity to a VEGF-C known to those of skill in the art (e.g., any one of SEQ ID NOs: 19-24, 41-46, 51, or 52). In another specific embodiment, a VEGF-C derivative has at least 90%, 95%, 96%, 98%, or 99% amino acid sequence identity to a VEGF-C known to those of skill in the art (e.g., any one of SEQ ID NOs: 19-24, 41-46, 51, or 52). In another specific embodiment, a VEGF-C derivative has at least 95%, 96%, 98%, or 99% amino acid sequence identity to a VEGF-C known to those of skill in the art (e.g., any one of SEQ ID NOs: 19-24, 41-46, 51, or 52). In another embodiment, a VEGF-C derivative has at least 85%, 90%, 95%, 96%, 98% or 99% identity to native human VEGF-C (e.g., SEQ ID NO: 41) or a fragment thereof (e.g., a fragment comprising the VEGF homology domain). In another embodiment, a VEGF-C derivative has at least 85%, 90%, 95%, 96%, 98% or 99% identity to native mature VEGF-C (e.g., SEQ ID NO: 44). In another embodiment, a VEGF-C derivative comprises a VEGF homology domain, wherein the VEGF homology domain has at least 85%, 90%, 95%, 96%, 98% or 99% identity to the VEGF homology domain of native VEGF-C. Methods/techniques known in the art may be used to determine sequence identity (see, e.g., “Best Fit” or “Gap” program of the Sequence Analysis Software Package, version 10; Genetics Computer Group, Inc.). In a specific embodiment, a VEGF-C derivative comprises deleted forms of a known VEGF-C (e.g., human VEGF-C), wherein up to about 20, 15, 10, 9, 8, 7, 6, 5, 4, 3, 2 or 1 amino acid residues are deleted from the known VEGF-C (e.g., human VEGF-C). Also provided herein are VEGF-C derivatives comprising deleted forms of a known VEGF-C, wherein about 1-3, 3-5, 5-7, 7-10, 10-15, or 15-20 amino acid residues are deleted from the known VEGF-C (e.g., human VEGF-C). Further provided herein are VEGF-C derivatives comprising altered forms of a known VEGF-C (e.g., human VEGF-C), wherein up to about 20, 15, 10, 9, 8, 7, 6, 5, 4, 3, 2 or 1 amino acid residues of the known VEGF-C are substituted (e.g., conservatively substituted) with other amino acids. In a specific embodiment, the known VEGF-C is human VEGF-C, such as, e.g., provided in GenBank™ accession number NM_005429.5, Uniprot P49767, or Uniprot Q6FH59. In some embodiments, a VEGF-C derivative comprises up to about 20, 15, 10, 9, 8, 7, 6, 5, 4, 3, 2 or 1 conservatively substituted amino acids. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophylic (Cys, Ser, Thr), acidic (Asp, Glu), basic (Asn, Gln, His, Lys, Arg), conformation disruptors (Gly, Pro) and aromatic (Trp, Tyr, Phe).

In a specific embodiment, a VEGF-C derivative is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a native VEGF-C (e.g., human VEGF-C). In a specific embodiment, a VEGF-C derivative is at least 90%, 95%, 98%, or 99% or is 90% to 95%, 90% to 99%, 95% to 98%, or 95% to 99% identical (e.g., sequence identity) to a native VEGF-C (e.g., human VEGF-C). In another specific embodiment, a VEGF-C derivative is a polypeptide encoded by a nucleic acid sequence that is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a nucleic acid sequence encoding a native VEGF-C (e.g., human VEGF-C). In another specific embodiment, a VEGF-C is a polypeptide encoded by a nucleic acid sequence that is at least 90%, 95%, 98%, or 99% or is 90% to 95%, 90% to 99%, 95% to 98%, or 95% to 99% identical (e.g., sequence identity) to a nucleic acid sequence encoding a native VEGF-C. In a specific embodiment, the native VEGF-C is human VEGF-C, such as, e.g., provided in GenBank™ accession number NM_005429.5, Uniprot P49767, or Uniprot Q6FH59. In some embodiments, the native VEGF-C is canine VEGF-C, such as, e.g., provided in GenBank™ accession numbers XM_S40047.6 and XM_02543044. In another specific embodiment, a VEGF-C derivative contains 1, 2, 3, 4, 5, 6, 7, 8, 9, 10, 11, 12, 13, 14, 15, 16, 17, 18, 19, 20 or more, or 2 to 5, 2 to 10, 5 to 10, 5 to 15, 5 to 20, 10 to 15, or 15 to 20 amino acid mutations (i.e., additions, deletions, substitutions or any combination thereof) relative to a native VEGF-C (e.g., human VEGF-C). In another specific embodiment, a VEGF-C derivative is a polypeptide encoded by nucleic acid sequence that can hybridize under high, moderate or typical stringency hybridization conditions to a nucleic acid sequence encoding a native VEGF-C (e.g., human VEGF-C). Hybridization conditions are known to one of skill in the art (see, e.g., U.S. Patent Application No. 2005/0048549 at, e.g., paragraphs 72 and 73). In another specific embodiment, a VEGF-C derivative is a polypeptide encoded by a nucleic acid sequence that can hybridize under high, moderate or typical stringency hybridization conditions to a nucleic acid sequence encoding a fragment of a native VEGF-C (e.g., human VEGF-C) of at least 10 contiguous amino acids, at least 12 contiguous amino acids, at least 15 contiguous amino acids, at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, at least 150 contiguous amino acids, or 10 to 20, 20 to 50, 25 to 75, 25 to 100, 25 to 150, 50 to 75, 50 to 100, 75 to 100, 50 to 150, 75 to 150, 100 to 150, or 100 to 200 contiguous amino acids. In another specific embodiment, a VEGF-C derivative is a fragment of a native VEGF-C (e.g., human VEGF-C). In another specific embodiment, a VEGF-C derivative comprises a fragment of human VEGF-C (e.g., a fragment of SEQ ID NO: 41 or 44). In a specific embodiment, a VEGF-C derivative is a fragment of a native VEGF-C (e.g., a human VEGF-C) that comprises the VEGF homology domain. In another specific embodiment, a VEGF-C derivative comprises a fragment of a human VEGF-C (e.g., SEQ ID NO: 41 or 44), wherein the fragment comprises the VEGF homology domain. In a specific embodiment, a fragment of native VEGF-C retains the ability to bind to VEGFR-3, induces phosphorylation of VEGFR-3 and induces downstream signaling events, such as, e.g., phosphorylation of serine/threonine kinases, such as, e.g., AKT, ERK 1/2, or Stat 3.

VEGF-C derivatives also include polypeptides that comprise the amino acid sequence of a naturally occurring mature form of VEGF-C and a heterologous amino acid sequence. VEGF-C derivatives also include polypeptides that comprise the amino acid sequence of a naturally occurring mature form of VEGF-C and a heterologous signal peptide amino acid sequence. In addition, VEGF-C derivatives include polypeptides that have been chemically modified by, e.g., glycosylation, acetylation, pegylation, phosphorylation, amidation, derivitization by known protecting/blocking groups, proteolytic cleavage, linkage to a cellular ligand or other protein moiety, etc. Further, VEGF-C derivatives include polypeptides comprising one or more non-classical amino acids.

In a specific embodiment, a VEGF-C derivative binds to VEGFR-3 and induces phosphorylation of VEGFR-3 and activates downstream signaling, e.g., phosphorylation of serine/threonine kinases, such as AKT and ERT1/2 and Stat3. In specific embodiments, the VEGF-C derivative retains one, two, or more, or all of the functions of the native VEGF-C (e.g., human VEGF-C) from which it was derived. Examples of functions of VEGF-C include lymphangiogenesis, lymphatic endothelial proliferation, migration, or activation, lymphatic permeability and contractility, angiogenesis, regulation of blood vessel permeability, endothelial cell growth, macrophage recruitment or modulation of function and immunomodulation. Tests for determining whether or not a VEGF-C derivative retains one or more functions of the native VEGF-C (e.g., human VEGF-C) from which it was derived are known to one of skill in the art and examples are provided herein. In a specific embodiment, a VEGF-C derivative binds to VEGFR-3 but not VEGFR-2. In certain embodiments, a VEGF-C derivative comprises the nucleotide sequence of SEQ ID NO: 2, 3, 5, 6, 8, 9, 11, 12, 14, 15, 17, 18, 30, 31, 33, 34, 36, 37, 39, or 40. In certain embodiments, a VEGF-C derivative comprises the nucleotide sequence of SEQ ID NO: 2, 3, 5, 6, 8, 9, 11, 12, 14, 15, 17, or 18. In certain embodiments, a VEGF-C derivative comprises the nucleotide sequence of SEQ ID NO: 30, 31, 33, 34, 36, 37, 39, or 40. In some embodiments, a VEGF-C derivative is mature VEGF-C Cys156Ser (e.g., SEQ ID NO: 45). In certain embodiments, a VEGF-C derivative is mature VEGF-C Cys137Ala (e.g., SEQ ID NO: 46).

In specific embodiments, the transgene comprising a nucleotide sequence encoding VEGF-C or a derivative thereof in a genome of a recombinant oncolytic virus described herein (e.g., APMV, such as APMV-1 or APMV-4) is codon optimized. In specific embodiments, a nucleotide sequence encoding VEGF-C or a derivative thereof in a genome of a recombinant oncolytic virus described herein (e.g., APMV, such as APMV-1 or APMV-4) is codon optimized.

VEGF-D

In a specific embodiment, a transgene comprising a nucleotide sequence encoding a VEGF-D agent is incorporated into the genome of an oncolytic virus described herein, such as an APMV (e.g., APMV-1 or APMV-4). See, e.g., Section 5.1. and Section 5.3, supra, for oncolytic viruses that may be used. A nucleotide sequence may encode precursor VEGF-D, pro-VEGF-D-ΔC, or mature VEGF-D. In one embodiment, a nucleotide sequence encodes a full-length form of VEGF-D. In some embodiments, a nucleotide sequence encodes unprocessed form of VEGF-D. In a specific embodiment, a nucleotide sequence encodes human VEGF-D. In another specific embodiment, human VEGF-D comprises the amino acid sequence set forth in SEQ ID NO:101-104. In another specific embodiment, the nucleic acid sequence encoding a human VEGF-D comprises the nucleotide sequence set forth in SEQ ID NO: 96. In another specific embodiment, a nucleotide sequence encodes canine VEGF-D. In another specific embodiment, canine VEGF-D comprises the amino acid sequence set forth in SEQ ID NO: 99 or 100. In another specific embodiment, the nucleic acid sequence encoding canine VEGF-D comprises the nucleotide sequence set forth in SEQ ID NO: 97 or 98.

In a specific embodiment, a transgene comprises a nucleotide sequence that encodes human VEGF-D. In one embodiment, a nucleotide sequence encodes the amino acid sequence set forth in SEQ ID NO: 101-104. In some embodiments, the transgene comprises the nucleotide sequence set forth in SEQ ID NO: 96. One of skill in the art would be able to use such sequence information to produce a transgene for incorporation into the genome of an oncolytic virus described herein, such as an APMV (e.g., APMV-1 or APMV-4). For example, a transgene encoding a human VEGF-D comprising the amino acid sequence set forth in Uniprot O43915 may be incorporated into the genome of any APMV type or strain described herein. In a specific embodiment, such a transgene comprises the sequence set forth in SEQ ID NO: 96. However, given the degeneracy of the nucleic acid code, there are a number of different nucleic acid sequences that may encode the same VEGF-D protein. In a specific embodiment, a transgene comprising a nucleotide sequence encoding VEGF-D (e.g., human VEGF-D) is codon optimized. See, e.g., Section 5.3.2.1, infra, for a discussion regarding codon optimization. In some embodiments, the transgene comprising a nucleotide sequence encoding a human VEGF-D protein comprises the amino acid sequence encoded by the nucleic acid sequence comprising the sequence set forth in SEQ ID NO: 96. The transgene encoding VEGF-D (e.g., human VEGF-D) may be incorporated between any two transcription units (e.g., between the APMV P and M transcription units, or between the HN and L transcription units).

In certain embodiments, the VEGF-D may be human, dog, cat, horse, pig, or cow VEGF-D. In a specific embodiment, the VEGF-D is human VEGF-D. Uniprot O43915 provides an exemplary human VEGF-D nucleic acid sequence. Uniprot O43915 provides an exemplary human VEGF-D amino acid sequence. In other embodiments, the native VEGF-D, is a canine VEGF-D, such as e.g, provided in GenBank™ numbers XM_548869.5 or XM_025437083. In specific embodiments, the VEGF-D proteins are modified by post-translational processing such as signal peptide cleavage, disulfide bond formation, glycosylation (e.g., N-linked glycosylation), protease cleavage and lipid modification (e.g., S-palmitoylation). In some embodiments, VEGF-D protein includes a signal sequence. In other embodiments, VEGF-D protein does not include a signal sequence. The signal sequence can be the naturally occurring signal peptide sequence or a variant thereof. In some embodiments, the signal peptide is a VEGF-D signal peptide. In some embodiments, the signal peptide is heterologous to a VEGF-D signal peptide.

In a specific embodiment, a transgene comprising a nucleotide sequence encoding a VEGF-D derivative is incorporated into the genome of an oncolytic virus described herein, such as an APMV (e.g., APMV-1 or APMV-4). See, e.g., Section 5.1 and Section 5.3.1, supra, for oncolytic viruses that may be used. In a specific embodiment, a transgene comprises a nucleotide sequence that encodes a human VEGF-D derivative. In another embodiment, a transgene comprises a nucleotide sequence that encodes a canine VEGF-D derivative. One of skill in the art would be able to use the sequence information to produce a transgene for incorporation into the genome of an comprising a nucleotide sequence. In a specific embodiment, a VEGF-D derivative has at least 70%, 75%, 80%, 85%, 90%, 95%, 96%, 98%, or 99% amino acid sequence identity to a VEGF-D known to those of skill in the art (e.g., SEQ ID NO: 99-104). In another specific embodiment, a VEGF-D derivative has at least 85%, 90%, 95%, 96%, 98%, or 99% amino acid sequence identity to a VEGF-D known to those of skill in the art (e.g., SEQ ID NO: 99-104). In another specific embodiment, a VEGF-D derivative has at least 90%, 95%, 96%, 98%, or 99% amino acid sequence identity to a VEGF-D known to those of skill in the art (e.g., SEQ ID NO: 99-104). In another specific embodiment, a VEGF-D derivative has at least 95%, 96%, 98%, or 99% amino acid sequence identity to a VEGF-D known to those of skill in the art (e.g., SEQ ID NO: 99-104). In another embodiment, a VEGF-D derivative has at least 85%, 90%, 95%, 96%, 98% or 99% identity to native human VEGF-D (e.g., SEQ ID NO: 104) or a fragment thereof (e.g., a fragment comprising the VEGF homology domain). In another embodiment, a VEGF-D derivative has at least 85%, 90%, 95%, 96%, 98% or 99% identity to native mature VEGF-D (e.g., SEQ ID NO: 101). In another embodiment, a VEGF-D derivative comprises a VEGF homology domain, wherein the VEGF homology domain has at least 85%, 90%, 95%, 96%, 98% or 99% identity to the VEGF homology domain of native VEGF-D. Methods/techniques known in the art may be used to determine sequence identity (see, e.g., “Best Fit” or “Gap” program of the Sequence Analysis Software Package, version 10; Genetics Computer Group, Inc.). In a specific embodiment, a VEGF-D derivative comprises deleted forms of a known VEGF-D (e.g., human VEGF-D), wherein up to about 20, 15, 10, 9, 8, 7, 6, 5, 4, 3, 2 or 1 amino acid residues are deleted from the known VEGF-D (e.g., human VEGF-D). Also provided herein are VEGF-D derivatives comprising deleted forms of a known VEGF-D, wherein about 1-3, 3-5, 5-7, 7-10, 10-15, or 15-20 amino acid residues are deleted from the known VEGF-D (e.g., human VEGF-D). Further provided herein are VEGF-D derivatives comprising altered forms of a known VEGF-D (e.g., human VEGF-D), wherein up to about 20, 15, 10, 9, 8, 7, 6, 5, 4, 3, 2 or 1 amino acid residues of the known VEGF-D are substituted (e.g., conservatively substituted) with other amino acids. In a specific embodiment, the known VEGF-D is human VEGF-D, such as, e.g., provided in Uniprot O43915. In other embodiments, the known VEGF-D is a canine VEGF-D, such as e.g, provided in GenBank™ numbers XM_548869.5 or XM_025437083. In some embodiments, a VEGF-D derivative comprises up to about 20, 15, 10, 9, 8, 7, 6, 5, 4, 3, 2 or 1 conservatively substituted amino acids. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophylic (Cys, Ser, Thr), acidic (Asp, Glu), basic (Asn, Gln, His, Lys, Arg), conformation disruptors (Gly, Pro) and aromatic (Trp, Tyr, Phe).

In a specific embodiment, a VEGF-D derivative is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a native VEGF-D (e.g., human VEGF-D). In a specific embodiment, a VEGF-D derivative is at least 90%, 95%, 98%, or 99% or is 90% to 95%, 90% to 99%, 95% to 98%, or 95% to 99% identical (e.g., sequence identity) to a native VEGF-D (e.g., human VEGF-D). In another specific embodiment, a VEGF-D derivative is a polypeptide encoded by a nucleic acid sequence that is at least 80%, 85%, 90%, 95%, 98%, or 99% or is 80% to 85%, 80% to 90%, 80% to 95%, 90% to 95%, 85% to 99%, or 95% to 99% identical (e.g., sequence identity) to a nucleic acid sequence encoding a native VEGF-D. In another specific embodiment, a VEGF-D is a polypeptide encoded by a nucleic acid sequence that is at least 90%, 95%, 98%, or 99% or is 90% to 95%, 90% to 99%, 95% to 98%, or 95% to 99% identical (e.g., sequence identity) to a nucleic acid sequence encoding a native VEGF-D. In a specific embodiment, the native VEGF-D is human VEGF-D, such as, e.g., provided in Uniprot O43915. In other embodiments, the native VEGF-D is a canine VEGF-D, such as e.g, provided in GenBank™ numbers XM_548869.5 or XM_025437083. In another specific embodiment, a VEGF-D derivative contains 1, 2, 3, 4, 5, 6, 7, 8, 9, 10, 11, 12, 13, 14, 15, 16, 17, 18, 19, 20 or more, or 2 to 5, 2 to 10, 5 to 10, 5 to 15, 5 to 20, 10 to 15, or 15 to 20 amino acid mutations (i.e., additions, deletions, substitutions or any combination thereof) relative to a native VEGF-D (e.g., human VEGF-D). In another specific embodiment, a VEGF-D derivative is a polypeptide encoded by nucleic acid sequence that can hybridize under high, moderate or typical stringency hybridization conditions to a nucleic acid sequence encoding a native VEGF-D (e.g., human VEGF-D). Hybridization conditions are known to one of skill in the art (see, e.g., U.S. Patent Application No. 2005/0048549 at, e.g., paragraphs 72 and 73). In another specific embodiment, a VEGF-D derivative is a polypeptide encoded by a nucleic acid sequence that can hybridize under high, moderate or typical stringency hybridization conditions to a nucleic acid sequence encoding a fragment of a native VEGF-D (e.g., human VEGF-D) of at least 10 contiguous amino acids, at least 12 contiguous amino acids, at least 15 contiguous amino acids, at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, at least 150 contiguous amino acids, or 10 to 20, 20 to 50, 25 to 75, 25 to 100, 25 to 150, 50 to 75, 50 to 100, 75 to 100, 50 to 150, 75 to 150, 100 to 150, or 100 to 200 contiguous amino acids. In another specific embodiment, a VEGF-D derivative comprises a fragment of human VEGF-D (e.g., a fragment of any one of SEQ ID NOs: 101-104). In another specific embodiment, a VEGF-D derivative is a fragment of a native VEGF-D (e.g., human VEGF-D). In a specific embodiment, a VEGF-D derivative is a fragment of a native VEGF-D (e.g., a human VEGF-D) that comprises the VEGF homology domain. In another specific embodiment, a VEGF-D derivative comprises a fragment of a human VEGF-D (e.g., a fragment of SEQ ID NO: 101-104), wherein the fragment comprises the VEGF homology domain. In a specific embodiment, a fragment of native VEGF-D retains the ability to bind to VEGFR-3, induces phosphorylation of VEGFR-3 and induces downstream signaling events, such as, e.g., phosphorylation of serine/threonine kinases, such as, e.g., AKT, ERK 1/2, or Stat 3.

VEGF-D derivatives also include polypeptides that comprise the amino acid sequence of a naturally occurring mature form of VEGF-D and a heterologous amino acid sequence. VEGF-D derivatives also include polypeptides that comprise the amino acid sequence of a naturally occurring mature form of VEGF-D and a heterologous signal peptide amino acid sequence. In addition, VEGF-D derivatives include polypeptides that have been chemically modified by, e.g., glycosylation, acetylation, pegylation, phosphorylation, amidation, derivitization by known protecting/blocking groups, proteolytic cleavage, linkage to a cellular ligand or other protein moiety, etc. Further, VEGF-D derivatives include polypeptides comprising one or more non-classical amino acids.

In a specific embodiment, a VEGF-D derivative binds to VEGFR-3 and induces phosphorylation of VEGFR-3 and activates downstream signaling, e.g., phosphorylation of serine/threonine kinases, such as AKT and ERT. In specific embodiments, the VEGF-D derivative retains one, two, or more, or all of the functions of the native VEGF-D (e.g., human VEGF-D) from which it was derived. Examples of functions of VEGF-D include lymphatic endothelial proliferation and migration, lymphatic permeability and contractility, angiogenesis, and remodeling of lymphatic and blood vessels. Tests for determining whether or not a VEGF-D derivative retains one or more functions of the native VEGF-D (e.g., human VEGF-D, such as, e.g., SEQ ID NO: 101 or 104) from which it was derived are known to one of skill in the art and examples are provided herein. In a specific embodiment, a VEGF-D derivative binds to VEGFR-3 but not VEGFR-2.

In specific embodiments, the transgene comprising a nucleotide sequence encoding VEGF-D or a derivative thereof in a genome of a recombinant oncolytic virus described herein (e.g., APMV, such as APMV-1 or APMV-4) is codon optimized. In specific embodiments, a nucleotide sequence encoding VEGF-D or a derivative thereof in a genome of a recombinant oncolytic virus described herein (e.g., APMV, such as APMV-1 or APMV-4) is codon optimized.

5.3.2.1 Codon Optimization

Any codon optimization technique known to one of skill in the art may be used to codon optimize a nucleic acid sequence encoding a VEGFR-3-activating agent, VEGF-C agent or a VEGF-D agent. Methods of codon optimization are known in the art, e.g, the OptimumGene™ (GenScript®) protocol and Genewiz® protocol, which are incorporated by reference herein in its entirety. See also U.S. Pat. No. 8,326,547 for methods for codon optimization, which is incorporated herein by reference in its entirety.

As an exemplary method for codon optimization, each codon in the open frame of the nucleic acid sequence encoding a VEGF-C agent or a VEGF-D agent is replaced by the codon most frequently used in mammalian proteins. This may be done using a web-based program (www.encorbio.com/protocols/Codon.htm) that uses the Codon Usage Database, maintained by the Department of Plant Gene Research in Kazusa, Japan. This nucleic acid sequence optimized for mammalian expression may be inspected for: (1) the presence of stretches of 5×A or more that may act as transcription terminators; (2) the presence of restriction sites that may interfere with subcloning; and (3) compliance with the rule of six for viruses, such as APMV, that follow the rule of six. Following inspection, (1) stretches of 5×A or more that may act as transcription terminators may be replaced by synonymous mutations; (2) restriction sites that may interfere with subcloning may be replaced by synonymous mutations; (3) a virus's regulatory signals, such as APMV regulatory signals (gene end, intergenic and gene start sequences), and Kozak sequences for optimal protein expression may be added; and (4) nucleotides may be added in the non-coding region to ensure compliance with the rule of six for viruses, such as APMV, that follow the rule of six. Synonymous mutations are typically nucleotide changes that do not change the amino acid encoded. For example, in the case of a stretch of 6 As (AAAAAA), which sequence encodes Lys-Lys, a synonymous sequence would be AAGAAG, which sequence also encodes Lys-Lys.

### 5.4 Construction of Oncolytic Viruses

Techniques known to one of skill in the art may be used to generate/produce an oncolytic virus described herein. See, e.g., Pleschka et al., 1996, J. Virol. 70(6): 4188-4192; Neumann et al., 2005, PNAS 102(46) 16825-16829; Anderson et al., 2000, Gene Therapy 7: 1034-1038; Goins et al., 2008, Methods Mol. Biol. 433: 97-113; Ruedas et al., 2017, Methods Mol. Biol. 1581: 203-222; Hruby, 1990, Clin. Microbiol. Rev. 3(2): 153-170; Mura et al., npj Vaccines 4: 12; Pfaller et al., 2015, Virology 479-480: 331-344; Stanway et al., 1986, J. Virology 57: 1187-1190; Kaptein et al., 1997, Gene Therapy 4: 172-176 for examples of techniques known in the art for the generation/production of an oncolytic virus described herein.

Methods for cloning a recombinant oncolytic virus to encode a transgene and express a heterologous protein encoded by the transgene are known to one skilled in the art, such as, e.g., insertion of the transgene into a restriction site that has been engineered into the viral genome, inclusion appropriate signals in the transgene for recognition by the virus, and inclusion of a valid Kozak sequence (e.g., to improve eukaryotic ribosomal translation).

### 5.4.1 Construction of APMVs

APMVs and other negative-sense single-stranded RNA viruses (see, e.g., Sections 5.1, 5.3 and 6) can be generated using the reverse genetics technique. The reverse genetics technique involves the preparation of synthetic recombinant viral RNAs that contain the non-coding regions of the negative-strand, viral RNA which are essential for the recognition by viral polymerases and for packaging signals necessary to generate a mature virion. The recombinant RNAs are synthesized from a recombinant DNA template and reconstituted in vitro with purified viral polymerase complex to form recombinant ribonucleoproteins (RNPs) which can be used to transfect cells. A more efficient transfection is achieved if the viral polymerase proteins are present during transcription of the synthetic RNAs either in vitro or in vivo. The synthetic recombinant RNPs can be rescued into infectious virus particles. The foregoing techniques are described in U.S. Pat. No. 5,166,057 issued Nov. 24, 1992; in U.S. Pat. No. 5,854,037 issued Dec. 29, 1998; in U.S. Pat. No. 6,146,642 issued Nov. 14, 2000; in European Patent Publication EP 0702085A1, published Feb. 20, 1996; in U.S. patent application Ser. No. 09/152,845; in International Patent Publications PCT WO97/12032 published Apr. 3, 1997; WO96/34625 published Nov. 7, 1996; in European Patent Publication EP A780475; WO 99/02657 published Jan. 21, 1999; WO 98/53078 published Nov. 26, 1998; WO 98/02530 published Jan. 22, 1998; WO 99/15672 published Apr. 1, 1999; WO 98/13501 published Apr. 2, 1998; WO 97/06270 published Feb. 20, 1997; and EPO 780 475A1 published Jun. 25, 1997, each of which is incorporated by reference herein in its entirety.

The helper-free plasmid technology can also be utilized to engineer an APMV and other negative-sense single-stranded RNA viruses. In particular, helper-free plasmid technology can be utilized to engineer a recombinant APMV and other negative-sense single-stranded RNA viruses. Briefly, a complete cDNA of an APMV (e.g., an APMV-4 strain) is constructed, inserted into a plasmid vector and engineered to contain a unique restriction site between two transcription units (e.g., the APMV P and M transcription units; or the APMV HN and L transcription units). A nucleotide sequence encoding a heterologous amino acid sequence (e.g., a transgene or other sequence) may be inserted into the viral genome at the unique restriction site. Alternatively, a nucleotide sequence encoding a heterologous amino acid sequence (e.g., a transgene or other sequence) may be engineered into an APMV transcription unit so long as the insertion does not affect the ability of the virus to infect and replicate. The single segment is positioned between a T7 promoter and the hepatitis delta virus ribozyme to produce an exact negative or positive transcript from the T7 polymerase. The plasmid vector and expression vectors comprising the necessary viral proteins are transfected into cells leading to production of recombinant viral particles (see, e.g., International Publication No. WO 01/04333; U.S. Pat. Nos. 7,442,379, 6,146,642, 6,649,372, 6,544,785 and 7,384,774; Swayne et al. (2003). Avian Dis. 47:1047-1050; and Swayne et al. (2001). J. Virol. 11868-11873, each of which is incorporated by reference in its entirety). See also, e.g., Nolden et al., Scientific Reports 6: 23887 (2016) for reverse genetic techniques to generate negative-strand RNA viruses, which is incorporated herein by reference.

Bicistronic techniques to produce multiple proteins from a single mRNA are known to one of skill in the art. Bicistronic techniques allow the engineering of coding sequences of multiple proteins into a single mRNA through the use of IRES sequences. IRES sequences direct the internal recruitment of ribosomes to the RNA molecule and allow downstream translation in a cap independent manner. Briefly, a coding region of one protein is inserted downstream of the ORF of a second protein. The insertion is flanked by an IRES and any untranslated signal sequences necessary for proper expression and/or function. The insertion must not disrupt the open reading frame, polyadenylation or transcriptional promoters of the second protein (see, e.g., Garcia-Sastre et al., 1994, J. Virol. 68:6254-6261 and Garcia-Sastre et al., 1994 Dev. Biol. Stand. 82:237-246, each of which are incorporated by reference herein in their entirety).

Methods for cloning a recombinant APMV to encode a transgene and express a heterologous protein encoded by the transgene are known to one skilled in the art, such as, e.g., insertion of the transgene into a restriction site that has been engineered into the APMV genome, inclusion appropriate signals in the transgene for recognition by the APMV RNA-dependent-RNA polymerase (e.g., sequences upstream of the open reading frame of the transgene that allow for the APMV polymerase to recognize the end of the previous gene and the beginning of the transgene, which may be, e.g., spaced by a single nucleotide intergenic sequence), inclusion of a valid Kozak sequence (e.g., to improve eukaryotic ribosomal translation); incorporation of a transgene that satisfies the “rule of six” for APMV cloning; and inclusion of silent mutations to remove extraneous gene end and/or gene start sequences within the transgene. Regarding the Rule of Six, one skilled in the art will understand that efficient replication of APMV (and more generally, most members of the paramyxoviridae family) is dependent on the genome length being a multiple of six, known as the “rule of six” (see, e.g., Calain, P. & Roux, L. The rule of six, a basic feature of efficient replication of Sendai virus defective interfering RNA. J. Virol. 67, 4822-4830 (1993)). Thus, when constructing a recombinant APMV described herein, care should be taken to satisfy the “Rule of Six” for APMV cloning. Methods known to one skilled in the art to satisfy the Rule of Six for APMV cloning may be used, such as, e.g., addition of nucleotides downstream of the transgene. See, e.g., Ayllon et al., Rescue of Recombinant Newcastle Disease Virus from cDNA. J. Vis. Exp. (80), e50830, doi:10.3791/50830 (2013) for a discussion of methods for cloning and rescuing of APMV (e.g., a recombinant APMV), which is incorporated by reference herein in its entirety.

### 5.5 Propagation of Oncolytic Viruses

An oncolytic virus described herein, such as an APMV described herein (e.g., a naturally occurring APMV or a recombinant APMV; see, also, e.g., Sections 5.1, 5.3, and 6) can be propagated in any substrate that allows the virus to grow to titers that permit the uses of the viruses described herein. In one embodiment, the substrate allows the oncolytic virus described herein, such as an APMV described herein (e.g., a naturally occurring APMV or a recombinant APMV; see, also, e.g., Sections 5.1, 5.3, and 6). In a specific embodiment, the substrate allows the oncolytic virus described herein, such as an APMV described herein (e.g., a naturally occurring APMV or a recombinant APMV; see, also, e.g., Sections 5.1, 5.3, and 6), to grow to titers comparable to those determined for the corresponding wild-type viruses.

An oncolytic virus described herein, such as an APMV described herein (e.g., a naturally occurring APMV or a recombinant APMV; see, also, e.g., Sections 5.1, 5.3, and 6), may be grown in cells (e.g., avian cells, chicken cells, etc.) that are susceptible to infection by the viruses, embryonated eggs (e.g., chicken eggs or quail eggs) or animals (e.g., birds). Such methods are well-known to those skilled in the art. In a specific embodiment, an oncolytic virus described herein, such as an APMV described herein (e.g., a naturally occurring APMV or a recombinant APMV; see, also, e.g., Sections 5.1, 5.3, and 6), may be propagated in cancer cells, e.g., carcinoma cells (e.g., breast cancer cells and prostate cancer cells), sarcoma cells, leukemia cells, lymphoma cells, and germ cell tumor cells (e.g., testicular cancer cells and ovarian cancer cells). In another specific embodiment, an oncolytic virus described herein, such as an APMV described herein (e.g., a naturally occurring APMV or a recombinant APMV; see, also, e.g., Sections 5.1, 5.3, and 6), may be propagated in a cell line, e.g., cancer cell lines such as HeLa cells, MCF7 cells, B16-F10 cells, CT26 cells, TC-1 cells, THP-1 cells, U87 cells, DU145 cells, Lncap cells, and T47D cells. In certain embodiments, the cells or cell lines (e.g., cancer cells or cancer cell lines) are obtained and/or derived from a human(s). In another embodiment, an oncolytic virus described herein, such as an APMV described herein (e.g., a naturally occurring APMV or a recombinant APMV; see, also, e.g., Sections 5.1, 5.3, and 6), is propagated in chicken cells or embryonated eggs. Representative chicken cells include, but are not limited to, chicken embryo fibroblasts and chicken embryo kidney cells. In a specific embodiment, an oncolytic virus described herein, such as an APMV described herein (e.g., a naturally occurring APMV or a recombinant APMV; see, also, e.g., Sections 5.1, 5.3, and 6), is propagated in IFN-deficient cells (e.g., IFN-deficient cell lines). In a specific embodiment, an oncolytic virus described herein, such as an APMV described herein (e.g., a naturally occurring APMV or a recombinant APMV; see, also, e.g., Sections 5.1, 5.3, and 6), is propagated in Vero cells. In another specific embodiment, an oncolytic virus described herein, such as an APMV described herein (e.g., a naturally occurring APMV or a recombinant APMV; see, also, e.g., Sections 5.1, 5.3, and 6), is propagated in cancer cells. In another specific embodiment, an oncolytic virus described herein, such as an APMV described herein (e.g., a naturally occurring APMV or a recombinant APMV; see, also, e.g., Sections 5.1, 5.3, and 6), is propagated in chicken eggs or quail eggs. In certain embodiments, an oncolytic virus described herein, such as an APMV described herein (e.g., a naturally occurring APMV or a recombinant APMV; see, also, e.g., Sections 5.1, 5.3, and 6), is first propagated in embryonated eggs and then propagated in cells (e.g., a cell line).

An oncolytic virus described herein, such as an APMV described herein (e.g., a naturally occurring APMV or a recombinant APMV; see, also, e.g., Sections 5.1, 5.3, and 6), may be propagated in embryonated eggs, e.g., from 6 to 14 days old, 6 to 12 days old, 6 to 10 days old, 6 to 9 days old, 6 to 8 days old, 8 days old, 9 days old, 10 days old, 8 to 10 days old, 12 days old, or 10 to 12 days old. Young or immature embryonated eggs can be used to propagate an oncolytic virus described herein, such as an APMV described herein (e.g., a naturally occurring APMV or a recombinant APMV; see, also, e.g., Sections 5.1, 5.3, and 6). Immature embryonated eggs encompass eggs which are less than ten day old eggs, e.g., eggs 6 to 9 days old or 6 to 8 days old that are IFN-deficient. Immature embryonated eggs also encompass eggs which artificially mimic immature eggs up to, but less than ten day old, as a result of alterations to the growth conditions, e.g., changes in incubation temperatures; treating with drugs; or any other alteration which results in an egg with a retarded development, such that the IFN system is not fully developed as compared with ten to twelve day old eggs. In a specific embodiment, an oncolytic virus described herein, such as an APMV described herein (e.g., a naturally occurring APMV or a recombinant APMV; see, also, e.g., Sections 5.1, 5.3, and 6), are propagated in 8 or 9 day old embryonated chicken eggs. In another specific embodiment, an oncolytic virus described herein, such as an APMV described herein (e.g., a naturally occurring APMV or a recombinant APMV; see, also, e.g., Sections 5.1, 5.3, and 6), are propagated in 10 day old embryonated chicken eggs. An oncolytic virus described herein, such as an APMV described herein (e.g., a naturally occurring APMV or a recombinant APMV; see, also, e.g., Sections 5.1, 5.3, and 6), can be propagated in different locations of the embryonated egg, e.g., the allantoic cavity. For a detailed discussion on the growth and propagation viruses, see, e.g., U.S. Pat. Nos. 6,852,522 and 7,494,808, both of which are hereby incorporated by reference in their entireties.

In a specific embodiment, provided herein is a cell (e.g., a cell line) or embryonated egg (e.g., a chicken embryonated egg) comprising an oncolytic virus described herein, such as an APMV described herein (e.g., a naturally occurring APMV or a recombinant APMV; see, also, e.g., Sections 5.1, 5.3, and 6). Examples of cells as well as embryonated eggs which may comprise an oncolytic virus described herein, such as an APMV described herein, may be found above. In a specific embodiment, provided herein is a method for propagating an oncolytic virus described herein, such as an APMV described herein (e.g., a naturally occurring APMV or a recombinant APMV; see, also, e.g., Sections 5.1, 5.3, and 6), the method comprising culturing a substrate (e.g., a cell line or embryonated egg) infected with the virus. In another specific embodiment, provided herein is a method for propagating an oncolytic virus described herein, such as an APMV described herein (e.g., a naturally occurring APMV or a recombinant APMV; see, also, e.g., Sections 5.1, 5.3 and 6), the method comprising: (a) culturing a substrate (e.g., a cell line or embryonated egg) infected with the virus; and (b) isolating or purifying the virus from the substrate. In certain embodiments, these methods involve infecting the substrate with the oncolytic virus (such as an APMV described herein) prior to culturing the substrate. See, e.g., Section 6, infra, for methods that may be used to propagate an APMV described herein (e.g., a naturally occurring APMV or a recombinant APMV described herein).

For virus isolation, an oncolytic virus described herein, such as an APMV described herein (e.g., a naturally occurring APMV or a recombinant APMV; see, also, e.g., Sections 5.1, 5.3, and 6), can be removed from embryonated eggs or cell culture and separated from cellular components, typically by well-known clarification procedures, e.g., such as centrifugation, depth filtration, and microfiltration, and may be further purified as desired using procedures well known to those skilled in the art, e.g., tangential flow filtration (TFF), density gradient centrifugation, differential extraction, or chromatography.

In a specific embodiment, provided herein is a method for producing a pharmaceutical composition (e.g., an immunogenic composition) comprising an oncolytic virus described herein, such as APMV described herein (e.g., a naturally occurring APMV or a recombinant APMV; see, also, e.g., Sections 5.1, 5.3, and 6), the method comprising (a) propagating an oncolytic virus described herein, such as an APMV described herein (e.g., a naturally occurring APMV or a recombinant APMV; see, also, e.g., Sections 5.1, 5.3, and 6), in a cell (e.g., a cell line) or embyronated egg; and (b) isolating the virus from the cell or embyronated egg. The method may further comprise adding the oncolytic virus (e.g., APMV) to a container along with a pharmaceutically acceptable carrier.

In a specific embodiment, an oncolytic virus described herein, such as an APMV described herein (e.g., a naturally occurring APMV or a recombinant APMV; see, also, e.g., Sections 5.1, 5.3, and 6), is propagated, isolated, and/or purified according to a method described in Section 6. In a specific embodiment, an oncolytic virus described herein, such as an APMV described herein (e.g., a naturally occurring APMV or a recombinant APMV; see, also, e.g., Sections 5.1, 5.3, and 6), is either propagated, isolated, or purified, or any two or all of the foregoing, using a method described in Section 6.

### 5.6 Compositions and Routes of Administration

Encompassed herein is the use of an oncolytic virus described herein, such as an APMV described herein (e.g., a naturally occurring APMV or a recombinant APMV described herein), in compositions. Also encompassed herein is the use of a VEGF-C agent or a VEGF-D agent in compositions. In a specific embodiment, the compositions are pharmaceutical compositions. The compositions may be used in methods of treating cancer.

In one embodiment, a pharmaceutical composition comprises an oncolytic virus described herein (e.g., a naturally occurring oncolytic virus or a recombinant oncolytic virus described herein), in an admixture with a pharmaceutically acceptable carrier. In some embodiments, the pharmaceutical composition further comprises one or more additional prophylactic or therapeutic agents, such as described in Section 5.7.2, infra. In a specific embodiment, a pharmaceutical composition comprises an effective amount of an oncolytic virus described herein (e.g., a naturally occurring oncolytic virus or a recombinant oncolytic virus described herein), and optionally one or more additional prophylactic or therapeutic agents, in a pharmaceutically acceptable carrier. In some embodiments, an oncolytic virus described herein (e.g., a naturally occurring oncolytic virus or a recombinant oncolytic virus described herein) is the only active ingredient included in the pharmaceutical composition. In certain embodiments, a pharmaceutical composition comprising an oncolytic virus described herein (e.g., a naturally occurring oncolytic virus or a recombinant oncolytic virus described herein) does not comprise an antigen (e.g., a bacterial antigen, a viral antigen, a fungal antigen, a protozoal antigen, a helminth antigen or a cancer antigen).

In another embodiment, a pharmaceutical composition comprises an APMV described herein (e.g., a naturally occurring APMV or a recombinant APMV described herein), in an admixture with a pharmaceutically acceptable carrier. In a specific embodiment, the APMV is an APMV-4 described herein. In other embodiments, the APMV is an APMV-6, APMV-7, APMV-8 or APMV-9 described herein. In a specific embodiment, the APMV is a recombinant APMV described herein. In a particular embodiment, the APMV is a recombinant APMV-4 comprising a packaged genome, wherein the packaged genome comprises the negative sense RNA transcribed from the cDNA sequence set forth in SEQ ID NO: 88 or 90. In some embodiments, the pharmaceutical composition further comprises one or more additional prophylactic or therapeutic agents, such as described in Section 5.7.2, infra. In a specific embodiment, a pharmaceutical composition comprises an effective amount of an APMV described herein (e.g., a naturally occurring APMV or a recombinant APMV described herein), and optionally one or more additional prophylactic or therapeutic agents, in a pharmaceutically acceptable carrier. In some embodiments, an APMV described herein (e.g., a naturally occurring APMV or a recombinant APMV described herein) is the only active ingredient included in the pharmaceutical composition. In certain embodiments, a pharmaceutical composition comprising an APMV described herein (e.g., a naturally occurring APMV or a recombinant APMV described herein) does not further comprise an antigen (e.g., a cancer antigen, a bacterial antigen, a fungal antigen, a protozoal antigen, a viral antigen or a helminth antigen).

In another embodiment, a pharmaceutical composition (e.g., an oncolysate vaccine) comprises a protein concentrate or a preparation of plasma membrane fragments from oncolytic virus infected cancer cells, in an admixture with a pharmaceutically acceptable carrier. In some embodiments, the pharmaceutical composition further comprises one or more additional prophylactic or therapeutic agents, such as described in Section 5.7.2, infra. In another embodiment, a pharmaceutical composition (e.g., a whole cell vaccine) comprises cancer cells infected with an oncolytic virus, in an admixture with a pharmaceutically acceptable carrier. In some embodiments, the pharmaceutical composition further comprises one or more additional prophylactic or therapeutic agents, such as described in Section 5.7.2, infra.

In another embodiment, a pharmaceutical composition (e.g., an oncolysate vaccine) comprises a protein concentrate or a preparation of plasma membrane fragments from APMV infected cancer cells, in an admixture with a pharmaceutically acceptable carrier. In some embodiments, the pharmaceutical composition further comprises one or more additional prophylactic or therapeutic agents, such as described in Section 5.7.2, infra. In another embodiment, a pharmaceutical composition (e.g., a whole cell vaccine) comprises cancer cells infected with APMV, in an admixture with a pharmaceutically acceptable carrier. In some embodiments, the pharmaceutical composition further comprises one or more additional prophylactic or therapeutic agents, such as described in Section 5.7.2, infra.

In another embodiment, provided herein is a pharmaceutical composition comprising a VEGFR-3-activating agent in an admixture with a pharmaceutically acceptable carrier. In some embodiments, a VEGFR-3-activating agent described herein is the only active ingredient included in the pharmaceutical composition. In other embodiments, the pharmaceutical composition further comprises an oncolytic virus described herein, such as an APMV described herein. In some embodiments, the pharmaceutical composition further comprises one or more additional prophylactic or therapeutic agents, such as described in Section 5.7.2, infra. In certain embodiments, a pharmaceutical composition comprises a VEGFR-3-activating agent and one or more additional prophylactic or therapeutic agents, such as described in Section 5.7.2, infra. In some embodiments, a pharmaceutical composition comprises a VEGFR-3-activating agent, an oncolytic virus described herein, such as an APMV described herein, and one or more additional prophylactic or therapeutic agents, such as described in Section 5.7.2, infra. In certain embodiments, a pharmaceutical composition comprises a VEGFR-3-activating agent and an antigen (e.g., a cancer antigen, a bacterial antigen, a fungal antigen, a protozoal antigen, a viral antigen or a helminth antigen). In some embodiments, a pharmaceutical composition comprising a VEGFR-3-activating agent does not comprise an antigen (e.g., a cancer antigen, a bacterial antigen, a fungal antigen, a protozoal antigen, a viral antigen or a helminth antigen).

In some embodiments, a VEGFR-3-activating agent is encapsulated within, contained within, complexed to or otherwise associated with a liposome, micelle, or a lipid particle or nanoparticle. In some embodiments, a VEGFR-3-activating agent is encapsulated in the aqueous interior of a liposome, interspersed within the lipid bilayer of a liposome, attached to a liposome via a linking molecule that is associated with both the liposome and the polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. In some embodiments, a VEGFR-3-activating agent is encapsulated or associated with a hydrogel.

In some embodiments, a VEGFR-3-activating agent is not encapsulated within, contained within, complexed to or otherwise associated with a liposome, micelle, or a lipid particle or nanoparticle. In some embodiments, a VEGFR-3-activating agent is not encapsulated in the aqueous interior of a liposome, interspersed within the lipid bilayer of a liposome, attached to a liposome via a linking molecule that is associated with both the liposome and the polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. In some embodiments, a VEGFR-3-activating agent is not encapsulated or associated with a hydrogel.

In another embodiment, provided herein is a pharmaceutical composition comprising a VEGF-C agent in an admixture with a pharmaceutically acceptable carrier. In some embodiments, a VEGF-C agent described herein is the only active ingredient included in the pharmaceutical composition. In other embodiments, the pharmaceutical composition further comprises an oncolytic virus described herein, such as an APMV described herein. In some embodiments, the pharmaceutical composition further comprises one or more additional prophylactic or therapeutic agents, such as described in Section 5.7.2, infra. In certain embodiments, a pharmaceutical composition comprises a VEGF-C agent and one or more additional prophylactic or therapeutic agents, such as described in Section 5.7.2, infra. In some embodiments, a pharmaceutical composition comprises a VEGF-C agent, an oncolytic virus described herein, such as an APMV described herein, and one or more additional prophylactic or therapeutic agents, such as described in Section 5.7.2, infra. In certain embodiments, a pharmaceutical composition comprises a VEGF-C agent and an antigen (e.g., a cancer antigen, a bacterial antigen, a fungal antigen, a protozoal antigen, a viral antigen or a helminth antigen). In some embodiments, a pharmaceutical composition comprising a VEGF-C agent does not comprise an antigen (e.g., a cancer antigen, a bacterial antigen, a fungal antigen, a protozoal antigen, a viral antigen or a helminth antigen).

In some embodiments, a VEGF-C agent is encapsulated within, contained within, complexed to or otherwise associated with a liposome, micelle, or a lipid particle or nanoparticle. In some embodiments, a VEGF-C agent is encapsulated in the aqueous interior of a liposome, interspersed within the lipid bilayer of a liposome, attached to a liposome via a linking molecule that is associated with both the liposome and the polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. In some embodiments, a VEGF-C agent is encapsulated or associated with a hydrogel.

In some embodiments, a VEGF-C agent is not encapsulated within, contained within, complexed to or otherwise associated with a liposome, micelle, or a lipid particle or nanoparticle. In some embodiments, a VEGF-C agent is not encapsulated in the aqueous interior of a liposome, interspersed within the lipid bilayer of a liposome, attached to a liposome via a linking molecule that is associated with both the liposome and the polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. In some embodiments, a VEGF-C agent is not encapsulated or associated with a hydrogel.

In another embodiment, provided herein is a pharmaceutical composition comprising a VEGF-D agent in an admixture with a pharmaceutically acceptable carrier. In some embodiments, a VEGF-D agent described herein is the only active ingredient included in the pharmaceutical composition. In other embodiments, the pharmaceutical composition further comprises an oncolytic virus described herein, such as an APMV described herein. In certain embodiments, a pharmaceutical composition comprises a VEGF-D agent and one or more additional prophylactic or therapeutic agents, such as described in Section 5.7.2, infra. In some embodiments, a pharmaceutical composition comprises a VEGF-D agent, an oncolytic virus described herein, such as an APMV described herein, and one or more additional prophylactic or therapeutic agents, such as described in Section 5.7.2, infra. In certain embodiments, a pharmaceutical composition comprises a VEGF-D agent and an antigen (e.g., a cancer antigen, a bacterial antigen, a fungal antigen, a protozoal antigen, a viral antigen or a helminth antigen). In some embodiments, a pharmaceutical composition comprising a VEGF-D agent does not comprise an antigen (e.g., a cancer antigen, a bacterial antigen, a fungal antigen, a protozoal antigen, a viral antigen or a helminth antigen).

In some embodiments, a VEGF-D agent is encapsulated within, contained within, complexed to or otherwise associated with a liposome, micelle, or a lipid particle or composition, or nanoparticle. In some embodiments, a VEGF-D agent is encapsulated in the aqueous interior of a liposome, interspersed within the lipid bilayer of a liposome, attached to a liposome via a linking molecule that is associated with both the liposome and the polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. In some embodiments, a VEGF-D agent is encapsulated or associated with a hydrogel.

In some embodiments, a VEGF-D agent is not encapsulated within, contained within, complexed to or otherwise associated with a liposome, micelle, or a lipid particle or composition, or nanoparticle. In some embodiments, a VEGF-D agent is not encapsulated in the aqueous interior of a liposome, interspersed within the lipid bilayer of a liposome, attached to a liposome via a linking molecule that is associated with both the liposome and the polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. In some embodiments, a VEGF-C agent is not encapsulated or associated with a hydrogel.

In another embodiment, provided herein is a pharmaceutical composition comprising a VEGF-C agent and a VEGF-D agent in an admixture with a pharmaceutically acceptable carrier. In some embodiments, a VEGF-C agent described herein and a VEGF-D agent are the only active ingredient included in the pharmaceutical composition. In other embodiments, the pharmaceutical composition further comprises an oncolytic virus described herein, such as an APMV described herein. In some embodiments, the pharmaceutical composition further comprises one or more additional prophylactic or therapeutic agents, such as described in Section 5.7.2, infra. In certain embodiments, a pharmaceutical composition comprises a VEGF-C agent, a VEGF-D agent and one or more additional prophylactic or therapeutic agents, such as described in Section 5.7.2, infra. In some embodiments, a pharmaceutical composition comprises a VEGF-C agent, a VEGF-D agent, an oncolytic virus described herein, such as an APMV described herein, and one or more additional prophylactic or therapeutic agents, such as described in Section 5.7.2, infra. In certain embodiments, a pharmaceutical composition comprising a VEGF-C agent and a VEGF-D agent further comprises an antigen (e.g., a cancer antigen, a bacterial antigen, a fungal antigen, a protozoal antigen, a viral antigen or a helminth antigen). In some embodiments, a pharmaceutical composition comprising a VEGF-C agent and a VEGF-D agent does not comprise an antigen (e.g., a cancer antigen, a bacterial antigen, a fungal antigen, a protozoal antigen, a viral antigen or a helminth antigen).

In some embodiments, a VEGF-C agent and a VEGF-D are encapsulated within, contained within, complexed to or otherwise associated with a liposome, micelle, or a lipid particle or composition, or nanoparticle. In some embodiments, a VEGF-C agent and a VEGF-D agent are encapsulated in the aqueous interior of a liposome, interspersed within the lipid bilayer of a liposome, attached to a liposome via a linking molecule that is associated with both the liposome and the polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. In some embodiments, a VEGF-C agent and a VEGF-D agent are encapsulated or associated with a hydrogel.

In some embodiments, a VEGF-C agent and a VEGF-D are not encapsulated within, contained within, complexed to or otherwise associated with a liposome, micelle, or a lipid particle or composition, or nanoparticle. In some embodiments, a VEGF-C agent and a VEGF-D agent are not encapsulated in the aqueous interior of a liposome, interspersed within the lipid bilayer of a liposome, attached to a liposome via a linking molecule that is associated with both the liposome and the polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. In some embodiments, a VEGF-C agent and a VEGF-D agent are not encapsulated or associated with a hydrogel.

In another embodiment, any one or more of the additional therapies disclosed in Section 5.7.2 may also be provided as a pharmaceutical composition. For example, a pharmaceutical composition may contain polyI:C in an admixture with a pharmaceutically acceptable carrier.

The pharmaceutical compositions provided herein can be in any form that allows for the composition to be administered to a subject in need thereof. In a specific embodiment, the pharmaceutical compositions are suitable for veterinary administration, human administration or both. As used herein, the term “pharmaceutically acceptable” means approved by a regulatory agency of the Federal or a state government or listed in the U.S. Pharmacopeia or other generally recognized pharmacopeias for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. The formulation should suit the mode of administration.

In some embodiments, a pharmaceutical composition described herein comprises an adjuvant. In other embodiments, a pharmaceutical composition described herein does not comprise an adjuvant. An adjuvant may be poly IC or poly ICLC, TLR3 ligand, or a cytokine.

In a specific embodiment, the pharmaceutical compositions are formulated to be suitable for the intended route of administration to a subject. The pharmaceutical composition may be formulated for systemic or local administration to a subject. For example, the pharmaceutical composition may be formulated to be suitable for parenteral, intravenous, intraarterial, intrapleural, inhalation, intraperitoneal, oral, intradermal, colorectal, intraperitoneal, intracranial, and intratumoral administration. In a specific embodiment, the pharmaceutical composition may be formulated for intravenous, intraarterial, oral, intraperitoneal, intranasal, intradermal, intratracheal, intrapleural, intracranial, subcutaneous, intramuscular, topical, pulmonary, or intratumoral administration.

In a specific embodiment, a pharmaceutical composition comprising an oncolytic virus described herein (e.g., a naturally occurring oncolytic virus or a recombinant oncolytic virus described herein) is formulated to be suitable for intratumoral administration to the subject (e.g., human subject). In a specific embodiment, a pharmaceutical composition comprising an APMV described herein (e.g., a naturally occurring APMV or a recombinant APMV described herein) is formulated to be suitable for intratumoral administration to the subject (e.g., human subject). In a specific embodiment, a pharmaceutical composition comprising an APMV-1 described herein is formulated for intratumoral administration to a subject (e.g., a human subject). In a specific embodiment, a pharmaceutical composition comprising an APMV-4 described herein is formulated for intratumoral administration to a subject (e.g., a human subject). In other specific embodiments, a pharmaceutical composition comprising an APMV-2, APMV-3, APMV-5, APMV-6, APMV-7, APMV-8 or APMV-9 described herein is formulated for intratumoral administration to a subject (e.g., a human subject). In another specific embodiment, a pharmaceutical composition comprising a recombinant APMV described herein is formulated for intratumoral administration to the subject (e.g., human subject).

In a specific embodiment, a pharmaceutical composition comprising an oncolytic virus described herein (e.g., a naturally occurring oncolytic virus or a recombinant oncolytic virus described herein) is formulated to be suitable for intravenous administration to the subject (e.g., human subject). In a specific embodiment, a pharmaceutical composition comprising an APMV described herein (e.g., a naturally occurring APMV or a recombinant APMV described herein) is formulated to be suitable for intravenous administration to the subject (e.g., human subject). In a specific embodiment, a pharmaceutical composition comprising an APMV-1 described herein is formulated for intravenous administration to a subject (e.g., a human subject). In a specific embodiment, a pharmaceutical composition comprising an APMV-4 described herein is formulated for intravenous administration to a subject (e.g., a human subject). In other specific embodiments, a pharmaceutical composition comprising an APMV-2, APMV-3, APMV-5, APMV-6, APMV-7, APMV-8 or APMV-9 described herein is formulated for intravenous administration to a subject (e.g., a human subject). In another specific embodiment, a pharmaceutical composition comprising a recombinant APMV described herein is formulated for intravenous administration to the subject (e.g., human subject).

To the extent an oncolytic virus described herein, such as an APMV described herein (e.g., a naturally occurring APMV or recombinant APMV described herein), is administered in combination with another therapy, the other therapy (e.g., a VEGF-C agent, VEGF-D agent or a prophylactic or therapeutic agent such as described in Section 5.7.2, infra) may be administered in a separate pharmaceutical composition. In other words, two separate pharmaceutical compositions may be administered to a subject to treat cancer—one pharmaceutical composition comprising an oncolytic virus described herein, such as an APMV described herein (e.g., a naturally occurring APMV or recombinant APMV described herein), in an admixture with a pharmaceutically acceptable carrier, and a second pharmaceutical composition comprising another therapy (such as, e.g., a VEGF-C agent, VEGF-D agent, or a prophylactic or therapeutic such as described in Section 5.7.2, infra) in an admixture with a pharmaceutically acceptable carrier. The two pharmaceutical composition may be formulated for the same route of administration to the subject (e.g., human subject) or different routes of administration to the subject (e.g., human subject). For example, the pharmaceutical composition comprising an oncolytic virus described herein, such as an APMV described herein, may be formulated for local administration to a tumor of a subject (e.g. a human subject), while the pharmaceutical composition comprising another therapy (such as, e.g., a VEGF-C agent, VEGF-D agent, or a prophylactic or therapeutic such as described in Section 5.5.2, infra) is formulated for systemic administration to the subject (e.g., human subject). In one specific example, the pharmaceutical composition comprising an oncolytic virus described herein, such as an APMV described herein, may be formulated for intratumoral administration to the subject (e.g., human subject), while the pharmaceutical composition comprising another therapy (such as, e.g., a VEGF-C agent, VEGF-D agent, or a prophylactic or therapeutic such as described in Section 5.7.2, infra) is formulated for intravenous administration, subcutaneous administration or another route of administration to the subject (e.g., human subject). In another example, the pharmaceutical composition comprising an oncolytic virus described herein, such as an APMV described herein, and the pharmaceutical composition comprising another therapy (such as, e.g., a VEGF-C agent, VEGF-D agent, or a prophylactic or therapeutic such as described in Section 5.7.2, infra) may both be formulated for intravenous administration to the subject (e.g., human subject). In another example, the pharmaceutical composition comprising an oncolytic virus described herein, such as an APMV described herein, and the pharmaceutical composition comprising another therapy (such as, e.g., a VEGF-C agent, VEGF-D agent, or a prophylactic or therapeutic such as described in Section 5.7.2, infra) may both be formulated for intratumoral administration to the subject (e.g., human subject). In certain embodiments, a pharmaceutical composition comprising a therapy (such as, e.g., a VEGF-C agent, VEGF-D agent, or a prophylactic or therapeutic such as described in Section 5.7.2, infra), which is used in combination with an oncolytic virus described herein, such as an APMV described herein, or a composition thereof, is formulated for administration by an approved route, such as described in the Physicians' Desk Reference 71^{st} ed (2017).

### 5.7 Uses of Oncolytic Virus

In one aspect, a virus described herein, such as an oncolytic virus described herein (e.g., a naturally occurring or recombinant APMV described herein) or a composition thereof, an oncolysate described herein or a composition thereof, or whole cell vaccine may be used in the treatment of cancer. In one embodiment, provided herein are methods for treating cancer, comprising administering to a subject in need thereof a recombinant virus described herein, such as a recombinant oncolytic virus described herein (e.g., a recombinant APMV described herein), or a composition thereof. In another embodiment, provided herein is a method for treating cancer, comprising administering to a subject in need thereof an effective amount of an oncolytic virus described herein, such as a recombinant APMV described herein, or a composition thereof. See Section 5.7.4 for the types of cancer that may be treated in accordance with the methods described herein, Section 5.7.3 for the types of patients that may be treated in accordance with the methods described herein, and Section 5.7.1 for exemplary dosages and regimens for treating cancer in accordance with the methods described herein. In a specific embodiment, provided herein are methods for treating cancer, comprising administering to a subject in need thereof a composition as described in Section 5.6 that comprises a oncolytic virus described herein, such as an APMV described herein.

In certain embodiments, provided herein is a method for treating cancer, comprising administering to a subject in need thereof a recombinant oncolytic virus described herein, such as recombinant APMV described herein, or a composition thereof and one or more additional therapies, such as described in Section 5.7.2, infra. In a specific embodiment, provided herein is a method for treating cancer, comprising administering to a subject in need thereof an effective amount of a recombinant oncolytic virus described herein, such as a recombinant APMV described herein, or a composition thereof and an effective amount of one or more additional therapies, such as described in Section 5.7.2, infra. In a particular embodiment, one or more therapies are administered to a subject in combination with a recombinant oncolytic virus described herein, such as a recombinant APMV described herein, or a composition thereof to treat cancer. In a specific embodiment, the additional therapies are currently being used, have been used or are known to be useful in treating cancer. In another embodiment, a recombinant oncolytic virus described herein, such as a recombinant APMV described herein (e.g., a recombinant APMV described in Section 5.1, 5.3 or 6) or a composition thereof is administered to a subject in combination with a supportive therapy, a pain relief therapy, or other therapy that does not have a therapeutic effect on cancer. In certain embodiments, a recombinant oncolytic virus described herein, such as a recombinant APMV described herein, and one or more additional therapies are administered in the same composition. In other embodiments, a recombinant oncolytic virus described herein, such as a recombinant APMV described herein, and one or more additional therapies are administered in different compositions. A recombinant oncolytic virus described herein, such a recombinant APMV described herein, or a composition thereof in combination with one or more additional therapies, such as described herein in Section 5.7.2, infra, may be used as any line of therapy (e.g., a first, second, third, fourth or fifth line therapy) for treating cancer in accordance with a method described herein. See Section 5.7.4 for the types of cancer that may be treated in accordance with the methods described herein, Section 5.7.3 for the types of patients that may be treated in accordance with the methods described herein, and Section 5.7.1 for exemplary dosages and regimens for treating cancer in accordance with the methods described herein.

In another aspect, provided herein are methods for treating cancer, comprising administering to a subject in need thereof a virus described herein (e.g., an oncolytic virus described herein) and a VEGFR-3 activating agent. The VEGFR-3 activating agent and virus may be in the same composition or different compositions, and such compositions may or may not include additional therapies, such as described in Section 5.7.2. In certain embodiments, the VEGFR-3 activating agent and oncolytic virus (e.g., APMV) are in the same composition. In other embodiments, the VEGFR-3 activating agent and oncolytic virus (e.g., APMV) are in different compositions. In another embodiment, provided herein are methods for treating cancer, comprising administering to a subject in need thereof a first composition comprising an oncolytic virus described herein (e.g., a naturally occurring or recombinant APMV described herein) and a second composition comprising a VEGFR-3 activating agent. In a specific embodiment, provided herein is a method for treating cancer, comprising administering to a subject in need thereof an effective amount of an oncolytic virus described herein (e.g., a naturally occurring or recombinant APMV described herein) and an effective amount of a VEGFR-3 activating agent. In a specific embodiment, provided herein is a method for treating cancer, comprising administering to a subject in need thereof an effective amount of a first composition comprising an oncolytic virus described herein (e.g., a naturally occurring or recombinant APMV described herein) and an effective amount of a second composition comprising a VEGFR-3 activating agent. See Section 5.7.4 for the types of cancer that may be treated in accordance with the methods described herein, Section 5.7.3 for the types of patients that may be treated in accordance with the methods described herein, and Section 5.7.1 for exemplary dosages and regimens for treating cancer in accordance with the methods described herein.

In some embodiments, provided herein are methods for treating cancer, comprising administering to a subject in need thereof an oncolytic virus described herein (e.g., a naturally occurring or recombinant APMV described herein) and a VEGFR-3 activating agent, wherein the method does not involve administering to the subject an antigen (e.g., a bacterial antigen, a viral antigen, a fungal antigen, a protozoal antigen, a helminth antigen or a cancer antigen) or a composition thereof. For example, the oncolytic virus and VEGFR-3 activating agent are not administered to a subject in conjunction with an antigen (e.g., a bacterial antigen, a viral antigen, a fungal antigen, a protozoal antigen, a helminth antigen or a cancer antigen) or a composition thereof. In certain embodiments, the VEGFR-3 activating agent and oncolytic virus (e.g., APMV) are in the same composition. In other embodiments, the VEGFR-3 activating agent and oncolytic virus (e.g., APMV) are in different compositions. In some embodiments, the method further comprises administering VEGF-C or a composition thereof. In another embodiment, provided herein are methods for treating cancer, comprising administering to a subject in need thereof a first composition comprising an oncolytic virus described herein (e.g., a naturally occurring or recombinant APMV described herein) and a second composition comprising a VEGFR-3 activating agent, wherein the method does involve to the subject administering an antigen (e.g., a bacterial antigen, a viral antigen, a fungal antigen, a protozoal antigen, a helminth antigen or a cancer antigen) or a composition thereof. In a specific embodiment, provided herein is a method for treating cancer, comprising administering to a subject in need thereof an effective amount of an oncolytic virus described herein (e.g., a naturally occurring or recombinant APMV described herein) and an effective amount of a VEGFR-3 activating agent, wherein the method does involve administering to the subject an antigen (e.g., a bacterial antigen, a viral antigen, a fungal antigen, a protozoal antigen, a helminth antigen or a cancer antigen) or a composition thereof. In a specific embodiment, provided herein is a method for treating cancer, comprising administering to a subject in need thereof an effective amount of a first composition comprising an oncolytic virus described herein (e.g., a naturally occurring or recombinant APMV described herein) and an effective amount of a second composition comprising a VEGFR-3 activating agent, wherein the method does involve administering to the subject an antigen (e.g., a bacterial antigen, a viral antigen, a fungal antigen, a protozoal antigen, a helminth antigen or a cancer antigen) or a composition thereof. See Section 5.7.4 for the types of cancer that may be treated in accordance with the methods described herein, Section 5.7.3 for the types of patients that may be treated in accordance with the methods described herein, and Section 5.7.1 for exemplary dosages and regimens for treating cancer in accordance with the methods described herein.

In certain embodiments, provided herein are methods for treating cancer, comprising administering to a subject in need thereof an oncolytic virus described herein (e.g., a naturally occurring or recombinant APMV described herein) and a VEGFR-3 activating agent, wherein the method does not involve administering to the subject an additional active therapy (e.g., an additional active agent) to treat cancer. In certain embodiments, the VEGFR-3 activating agent and oncolytic virus (e.g., APMV) are in the same composition. In other embodiments, the VEGFR-3 activating agent and oncolytic virus (e.g., APMV) are in different compositions. In another embodiment, provided herein are methods for treating cancer, comprising administering to a subject in need thereof a first composition comprising an oncolytic virus described herein (e.g., a naturally occurring or recombinant APMV described herein) and a second composition comprising a VEGFR-3 activating agent, wherein the method does involve administering to the subject an additional active therapy (e.g., an additional active agent) to treat cancer. In a specific embodiment, provided herein is a method for treating cancer, comprising administering to a subject in need thereof an effective amount of an oncolytic virus described herein (e.g., a naturally occurring or recombinant APMV described herein) and an effective amount of a VEGFR-3 activating agent, wherein the method does involve administering to the subject an additional active therapy (e.g., an additional active agent) to treat cancer. In a specific embodiment, provided herein is a method for treating cancer, comprising administering to a subject in need thereof an effective amount of a first composition comprising an oncolytic virus described herein (e.g., a naturally occurring or recombinant APMV described herein) and an effective amount of a second composition comprising a VEGFR-3 activating agent, wherein the method does involve administering to the subject an additional active therapy (e.g., an additional active agent) to treat cancer. See Section 5.7.4 for the types of cancer that may be treated in accordance with the methods described herein, Section 5.7.3 for the types of patients that may be treated in accordance with the methods described herein, and Section 5.7.1 for exemplary dosages and regimens for treating cancer in accordance with the methods described herein.

In some embodiments, provided herein is a method for treating cancer, comprising administering to a subject in need thereof an oncolytic virus described herein, such as an APMV described herein (e.g., a naturally occurring or recombinant APMV described herein), or a composition thereof, a VEGFR-3 activating agent or a composition thereof, and one or more additional therapies, such as described in Section 5.7.2, infra. In a specific embodiment, provided herein is a method for treating cancer, comprising administering to a subject in need thereof an effective amount of an oncolytic virus described herein, such as an APMV described herein (e.g., a naturally occurring or recombinant APMV described herein), or a composition thereof, an effective amount of a VEGFR-3 activating agent or a composition thereof, and an effective amount of one or more additional therapies, such as described in Section 5.7.2, infra. In a particular embodiment, one or more therapies are administered to a subject in combination with an oncolytic virus described herein, such as an APMV described herein (e.g., a naturally occurring or recombinant APMV described herein), or a composition thereof and a VEGFR-3 activating agent or a composition thereof to treat cancer. In a specific embodiment, the additional therapies are currently being used, have been used or are known to be useful in treating cancer. In another embodiment, an oncolytic virus described herein, such as an APMV described herein (e.g., a naturally occurring or recombinant APMV described herein) or a composition thereof and a VEGFR-3 activating agent or a composition thereof are administered to a subject in combination with a supportive therapy, a pain relief therapy, or other therapy that does not have a therapeutic effect on cancer. In certain embodiments, an oncolytic virus described herein, such as an APMV described herein (e.g., a naturally occurring or recombinant APMV described herein), a VEGFR-3 activating agent and one or more additional therapies are administered in the same composition. In some embodiments, an oncolytic virus described herein, such as an APMV described herein (e.g., a naturally occurring or recombinant APMV described herein) is administered in a different composition from a VEGFR-3 activating agent and one or more additional therapies. In certain embodiments, an oncolytic virus described herein, such as an APMV described herein (e.g., a naturally occurring or recombinant APMV described herein) and one or more additional therapies are administered in the same composition. In some embodiments, an oncolytic virus described herein, such as an APMV described herein (e.g., a naturally occurring or recombinant APMV described herein) and one or more additional therapies are administered in different compositions. In certain embodiments, a VEGFR-3 activating agent and one or more additional therapies are administered in the same composition. In some embodiments, an oncolytic virus described herein, such as an APMV described herein (e.g., a naturally occurring or recombinant APMV described herein), and a VEGFR-3 activating agent and one or more additional therapies are each administered in different compositions. An oncolytic virus, such as an APMV described herein (e.g., a naturally occurring or recombinant APMV described herein), or a composition thereof in combination with a VEGFR-3 activating agent and one or more additional therapies, such as described herein in Section 5.7.2, infra, may be used as any line of therapy (e.g., a first, second, third, fourth or fifth line therapy) for treating cancer in accordance with a method described herein. See Section 5.7.4 for the types of cancer that may be treated in accordance with the methods described herein, Section 5.7.3 for the types of patients that may be treated in accordance with the methods described herein, and Section 5.7.1 for exemplary dosages and regimens for treating cancer in accordance with the methods described herein.

In another aspect, provided herein are methods for treating cancer, comprising administering to a subject in need thereof a virus described herein (e.g., an oncolytic virus described herein) and a VEGF-C agent. The VEGF-C agent and virus may be in the same composition or different compositions, and such compositions may or may not include additional therapies, such as described in Section 5.7.2. In one embodiment, provided herein are methods for treating cancer, comprising administering to a subject in need thereof an oncolytic virus described herein (e.g., a naturally occurring or recombinant APMV described herein) and a VEGF-C agent. In certain embodiments, the VEGF-C agent and oncolytic virus (e.g., APMV) are in the same composition. In other embodiments, the VEGF-C agent and oncolytic virus (e.g., APMV) are in different compositions. In some embodiments, the method further comprises administering a VEGF-D agent or a composition thereof. In another embodiment, provided herein are methods for treating cancer, comprising administering to a subject in need thereof a first composition comprising an oncolytic virus described herein (e.g., a naturally occurring or recombinant APMV described herein) and a second composition comprising a VEGF-C agent. In a specific embodiment, provided herein is a method for treating cancer, comprising administering to a subject in need thereof an effective amount of an oncolytic virus described herein (e.g., a naturally occurring or recombinant APMV described herein) and an effective amount of a VEGF-C agent. In a specific embodiment, provided herein is a method for treating cancer, comprising administering to a subject in need thereof an effective amount of a first composition comprising an oncolytic virus described herein (e.g., a naturally occurring or recombinant APMV described herein) and an effective amount of a second composition comprising a VEGF-C agent. See Section 5.7.4 for the types of cancer that may be treated in accordance with the methods described herein, Section 5.7.3 for the types of patients that may be treated in accordance with the methods described herein, and Section 5.7.1 for exemplary dosages and regimens for treating cancer in accordance with the methods described herein.

In some embodiments, provided herein are methods for treating cancer, comprising administering to a subject in need thereof an oncolytic virus described herein (e.g., a naturally occurring or recombinant APMV described herein) and a VEGF-C agent, wherein the method does not involve administering to the subject an antigen (e.g., a bacterial antigen, a viral antigen, a fungal antigen, a protozoal antigen, a helminth antigen or a cancer antigen) or a composition comprising antigen. For example, the oncolytic virus and VEGF-C agent are not administered to a subject in conjunction with an antigen (e.g., a bacterial antigen, a viral antigen, a fungal antigen, a protozoal antigen, a helminth antigen or a cancer antigen) or a composition thereof. In certain embodiments, the VEGF-C agent and oncolytic virus (e.g., APMV) are in the same composition. In other embodiments, the VEGF-C agent and oncolytic virus (e.g., APMV) are in different compositions. In some embodiments, the method further comprises administering a VEGF-D agent or a composition thereof. In another embodiment, provided herein are methods for treating cancer, comprising administering to a subject in need thereof a first composition comprising an oncolytic virus described herein (e.g., a naturally occurring or recombinant APMV described herein) and a second composition comprising a VEGF-C agent, wherein the method does involve administering to the subject an antigen (e.g., a bacterial antigen, a viral antigen, a fungal antigen, a protozoal antigen, a helminth antigen or a cancer antigen). In a specific embodiment, provided herein is a method for treating cancer, comprising administering to a subject in need thereof an effective amount of an oncolytic virus described herein (e.g., a naturally occurring or recombinant APMV described herein) and an effective amount of a VEGF-C agent, wherein the method does involve administering to the subject an antigen (e.g., a bacterial antigen, a viral antigen, a fungal antigen, a protozoal antigen, a helminth antigen or a cancer antigen). In a specific embodiment, provided herein is a method for treating cancer, comprising administering to a subject in need thereof an effective amount of a first composition comprising an oncolytic virus described herein (e.g., a naturally occurring or recombinant APMV described herein) and an effective amount of a second composition comprising a VEGF-C agent, wherein the method does involve administering to the subject an antigen (e.g., a bacterial antigen, a viral antigen, a fungal antigen, a protozoal antigen, a helminth antigen or a cancer antigen). See Section 5.7.4 for the types of cancer that may be treated in accordance with the methods described herein, Section 5.7.3 for the types of patients that may be treated in accordance with the methods described herein, and Section 5.7.1 for exemplary dosages and regimens for treating cancer in accordance with the methods described herein.

In certain embodiments, provided herein are methods for treating cancer, comprising administering to a subject in need thereof an oncolytic virus described herein (e.g., a naturally occurring or recombinant APMV described herein) and a VEGF-C agent, wherein the method does not involve administering to the subject an additional active therapy (e.g., an additional active agent) to treat cancer. In certain embodiments, the VEGF-C agent and oncolytic virus (e.g., APMV) are in the same composition. In other embodiments, the VEGF-C agent and oncolytic virus (e.g., APMV) are in different compositions. In another embodiment, provided herein are methods for treating cancer, comprising administering to a subject in need thereof a first composition comprising an oncolytic virus described herein (e.g., a naturally occurring or recombinant APMV described herein) and a second composition comprising a VEGF-C agent, wherein the method does involve administering to the subject an additional active therapy (e.g., an additional active agent) to treat cancer. In a specific embodiment, provided herein is a method for treating cancer, comprising administering to a subject in need thereof an effective amount of an oncolytic virus described herein (e.g., a naturally occurring or recombinant APMV described herein) and an effective amount of a VEGF-C agent, wherein the method does involve administering to the subject an additional active therapy (e.g., an additional active agent) to treat cancer. In a specific embodiment, provided herein is a method for treating cancer, comprising administering to a subject in need thereof an effective amount of a first composition comprising an oncolytic virus described herein (e.g., a naturally occurring or recombinant APMV described herein) and an effective amount of a second composition comprising a VEGF-C agent, wherein the method does involve administering to the subject an additional active therapy (e.g., an additional active agent) to treat cancer. See Section 5.7.4 for the types of cancer that may be treated in accordance with the methods described herein, Section 5.7.3 for the types of patients that may be treated in accordance with the methods described herein, and Section 5.7.1 for exemplary dosages and regimens for treating cancer in accordance with the methods described herein.

In some embodiments, provided herein is a method for treating cancer, comprising administering to a subject in need thereof an oncolytic virus described herein, such as an APMV described herein (e.g., a naturally occurring or recombinant APMV described herein), or a composition thereof, a VEGF-C agent or a composition thereof, and one or more additional therapies, such as described in Section 5.7.2, infra. In a specific embodiment, provided herein is a method for treating cancer, comprising administering to a subject in need thereof an effective amount of an oncolytic virus described herein, such as an APMV described herein (e.g., a naturally occurring or recombinant APMV described herein), or a composition thereof, an effective amount of a VEGF-C agent or a composition thereof, and an effective amount of one or more additional therapies, such as described in Section 5.7.2, infra. In a particular embodiment, one or more therapies are administered to a subject in combination with an oncolytic virus described herein, such as an APMV described herein (e.g., a naturally occurring or recombinant APMV described herein), or a composition thereof and a VEGF-C agent or a composition thereof to treat cancer. In a specific embodiment, the additional therapies are currently being used, have been used or are known to be useful in treating cancer. In another embodiment, an oncolytic virus described herein, such as an APMV described herein (e.g., a naturally occurring or recombinant APMV described herein) or a composition thereof and a VEGF-C agent or a composition thereof are administered to a subject in combination with a supportive therapy, a pain relief therapy, or other therapy that does not have a therapeutic effect on cancer. In certain embodiments, an oncolytic virus described herein, such as an APMV described herein (e.g., a naturally occurring or recombinant APMV described herein), a VEGF-C agent and one or more additional therapies are administered in the same composition. In some embodiments, an oncolytic virus described herein, such as an APMV described herein (e.g., a naturally occurring or recombinant APMV described herein) is administered in a different composition from a VEGF-C agent and one or more additional therapies. In certain embodiments, an oncolytic virus described herein, such as an APMV described herein (e.g., a naturally occurring or recombinant APMV described herein) and one or more additional therapies are administered in the same composition. In some embodiments, an oncolytic virus described herein, such as an APMV described herein (e.g., a naturally occurring or recombinant APMV described herein) and one or more additional therapies are administered in different compositions. In certain embodiments, a VEGF-C agent and one or more additional therapies are administered in the same composition. In some embodiments, an oncolytic virus described herein, such as an APMV described herein (e.g., a naturally occurring or recombinant APMV described herein), and a VEGF-C agent and one or more additional therapies are each administered in different compositions. An oncolytic virus, such as an APMV described herein (e.g., a naturally occurring or recombinant APMV described herein), or a composition thereof in combination with a VEGF-C agent and one or more additional therapies, such as described herein in Section 5.7.2, infra, may be used as any line of therapy (e.g., a first, second, third, fourth or fifth line therapy) for treating cancer in accordance with a method described herein. See Section 5.7.4 for the types of cancer that may be treated in accordance with the methods described herein, Section 5.7.3 for the types of patients that may be treated in accordance with the methods described herein, and Section 5.7.1 for exemplary dosages and regimens for treating cancer in accordance with the methods described herein.

In another aspect, provided herein are methods for treating cancer, comprising administering to a subject in need thereof a virus described herein (e.g., an oncolytic virus described herein) and a VEGF-C agent. The VEGF-C agent and virus may be in the same composition or different compositions, and such compositions may or may not include additional therapies, such as described in Section 5.7.2. In certain embodiments, the VEGF-D agent and oncolytic virus (e.g., APMV) are in the same composition. In other embodiments, the VEGF-D agent and oncolytic virus (e.g., APMV) are in different compositions. In some embodiments, the method further comprises administering a VEGF-C agent or a composition thereof. In another embodiment, provided herein are methods for treating cancer, comprising administering to a subject in need thereof a first composition comprising an oncolytic virus described herein (e.g., a naturally occurring or recombinant APMV described herein) and a second composition comprising a VEGF-D agent. In a specific embodiment, provided herein is a method for treating cancer, comprising administering to a subject in need thereof an effective amount of an oncolytic virus described herein (e.g., a naturally occurring or recombinant APMV described herein) and an effective amount of a VEGF-D agent. In a specific embodiment, provided herein is a method for treating cancer, comprising administering to a subject in need thereof an effective amount of a first composition comprising an oncolytic virus described herein (e.g., a naturally occurring or recombinant APMV described herein) and an effective amount of a second composition comprising a VEGF-D agent. See Section 5.7.4 for the types of cancer that may be treated in accordance with the methods described herein, Section 5.7.3 for the types of patients that may be treated in accordance with the methods described herein, and Section 5.7.1 for exemplary dosages and regimens for treating cancer in accordance with the methods described herein.

In some embodiments, provided herein are methods for treating cancer, comprising administering to a subject in need thereof an oncolytic virus described herein (e.g., a naturally occurring or recombinant APMV described herein) and a VEGF-D agent, wherein the method does not involve administering to the subject an antigen (e.g., a bacterial antigen, a viral antigen, a fungal antigen, a protozoal antigen, a helminth antigen or a cancer antigen) or a composition thereof. For example, the oncolytic virus and VEGF-D agent are not administered to a subject in conjunction with an antigen (e.g., a bacterial antigen, a viral antigen, a fungal antigen, a protozoal antigen, a helminth antigen or a cancer antigen) or a composition thereof. In certain embodiments, the VEGF-D agent and oncolytic virus (e.g., APMV) are in the same composition. In other embodiments, the VEGF-D agent and oncolytic virus (e.g., APMV) are in different compositions. In some embodiments, the method further comprises administering VEGF-C or a composition thereof. In another embodiment, provided herein are methods for treating cancer, comprising administering to a subject in need thereof a first composition comprising an oncolytic virus described herein (e.g., a naturally occurring or recombinant APMV described herein) and a second composition comprising a VEGF-D agent, wherein the method does involve to the subject administering an antigen (e.g., a bacterial antigen, a viral antigen, a fungal antigen, a protozoal antigen, a helminth antigen or a cancer antigen) or a composition thereof. In a specific embodiment, provided herein is a method for treating cancer, comprising administering to a subject in need thereof an effective amount of an oncolytic virus described herein (e.g., a naturally occurring or recombinant APMV described herein) and an effective amount of a VEGF-D agent, wherein the method does involve administering to the subject an antigen (e.g., a bacterial antigen, a viral antigen, a fungal antigen, a protozoal antigen, a helminth antigen or a cancer antigen) or a composition thereof. In a specific embodiment, provided herein is a method for treating cancer, comprising administering to a subject in need thereof an effective amount of a first composition comprising an oncolytic virus described herein (e.g., a naturally occurring or recombinant APMV described herein) and an effective amount of a second composition comprising a VEGF-D agent, wherein the method does involve administering to the subject an antigen (e.g., a bacterial antigen, a viral antigen, a fungal antigen, a protozoal antigen, a helminth antigen or a cancer antigen) or a composition thereof. See Section 5.7.4 for the types of cancer that may be treated in accordance with the methods described herein, Section 5.7.3 for the types of patients that may be treated in accordance with the methods described herein, and Section 5.7.1 for exemplary dosages and regimens for treating cancer in accordance with the methods described herein.

In certain embodiments, provided herein are methods for treating cancer, comprising administering to a subject in need thereof an oncolytic virus described herein (e.g., a naturally occurring or recombinant APMV described herein) and a VEGF-D agent, wherein the method does not involve administering to the subject an additional active therapy (e.g., an additional active agent) to treat cancer. In certain embodiments, the VEGF-D agent and oncolytic virus (e.g., APMV) are in the same composition. In other embodiments, the VEGF-D agent and oncolytic virus (e.g., APMV) are in different compositions. In another embodiment, provided herein are methods for treating cancer, comprising administering to a subject in need thereof a first composition comprising an oncolytic virus described herein (e.g., a naturally occurring or recombinant APMV described herein) and a second composition comprising a VEGF-D agent, wherein the method does involve administering to the subject an additional active therapy (e.g., an additional active agent) to treat cancer. In a specific embodiment, provided herein is a method for treating cancer, comprising administering to a subject in need thereof an effective amount of an oncolytic virus described herein (e.g., a naturally occurring or recombinant APMV described herein) and an effective amount of a VEGF-D agent, wherein the method does involve administering to the subject an additional active therapy (e.g., an additional active agent) to treat cancer. In a specific embodiment, provided herein is a method for treating cancer, comprising administering to a subject in need thereof an effective amount of a first composition comprising an oncolytic virus described herein (e.g., a naturally occurring or recombinant APMV described herein) and an effective amount of a second composition comprising a VEGF-D agent, wherein the method does involve administering to the subject an additional active therapy (e.g., an additional active agent) to treat cancer. See Section 5.7.4 for the types of cancer that may be treated in accordance with the methods described herein, Section 5.7.3 for the types of patients that may be treated in accordance with the methods described herein, and Section 5.7.1 for exemplary dosages and regimens for treating cancer in accordance with the methods described herein.

In some embodiments, provided herein is a method for treating cancer, comprising administering to a subject in need thereof an oncolytic virus described herein, such as an APMV described herein (e.g., a naturally occurring or recombinant APMV described herein), or a composition thereof, a VEGF-D agent or a composition thereof, and one or more additional therapies, such as described in Section 5.7.2, infra. In a specific embodiment, provided herein is a method for treating cancer, comprising administering to a subject in need thereof an effective amount of an oncolytic virus described herein, such as an APMV described herein (e.g., a naturally occurring or recombinant APMV described herein), or a composition thereof, an effective amount of a VEGF-D agent or a composition thereof, and an effective amount of one or more additional therapies, such as described in Section 5.7.2, infra. In a particular embodiment, one or more therapies are administered to a subject in combination with an oncolytic virus described herein, such as an APMV described herein (e.g., a naturally occurring or recombinant APMV described herein), or a composition thereof and a VEGF-D agent or a composition thereof to treat cancer. In a specific embodiment, the additional therapies are currently being used, have been used or are known to be useful in treating cancer. In another embodiment, an oncolytic virus described herein, such as an APMV described herein (e.g., a naturally occurring or recombinant APMV described herein) or a composition thereof and a VEGF-D agent or a composition thereof are administered to a subject in combination with a supportive therapy, a pain relief therapy, or other therapy that does not have a therapeutic effect on cancer. In certain embodiments, an oncolytic virus described herein, such as an APMV described herein (e.g., a naturally occurring or recombinant APMV described herein), a VEGF-D agent and one or more additional therapies are administered in the same composition. In some embodiments, an oncolytic virus described herein, such as an APMV described herein (e.g., a naturally occurring or recombinant APMV described herein) is administered in a different composition from a VEGF-D agent and one or more additional therapies. In certain embodiments, an oncolytic virus described herein, such as an APMV described herein (e.g., a naturally occurring or recombinant APMV described herein) and one or more additional therapies are administered in the same composition. In some embodiments, an oncolytic virus described herein, such as an APMV described herein (e.g., a naturally occurring or recombinant APMV described herein) and one or more additional therapies are administered in different compositions. In certain embodiments, a VEGF-D agent and one or more additional therapies are administered in the same composition. In some embodiments, an oncolytic virus described herein, such as an APMV described herein (e.g., a naturally occurring or recombinant APMV described herein), and a VEGF-D agent and one or more additional therapies are each administered in different compositions. An oncolytic virus, such as an APMV described herein (e.g., a naturally occurring or recombinant APMV described herein), or a composition thereof in combination with a VEGF-D agent and one or more additional therapies, such as described herein in Section 5.7.2, infra, may be used as any line of therapy (e.g., a first, second, third, fourth or fifth line therapy) for treating cancer in accordance with a method described herein. See Section 5.7.4 for the types of cancer that may be treated in accordance with the methods described herein, Section 5.7.3 for the types of patients that may be treated in accordance with the methods described herein, and Section 5.7.1 for exemplary dosages and regimens for treating cancer in accordance with the methods described herein.

In a specific embodiment, provided herein are methods for treating cancer, comprising administering to a subject in need thereof a first composition as described in Section 5.6 that comprises an oncolytic virus described herein, such as an APMV described herein, and a composition as described in Section 5.6 that comprises a VEGFR-3 activating agent. In another specific embodiment, provided herein are methods for treating cancer, comprising administering to a subject in need thereof a first composition as described in Section 5.6 that comprises a oncolytic virus described herein, such as an APMV described herein, and a composition as described in Section 5.6 that comprises a VEGF-C agent. In another specific embodiment, provided herein are methods for treating cancer, comprising administering to a subject in need thereof a first composition as described in Section 5.6 that comprises a oncolytic virus described herein, such as an APMV described herein, and a second composition as described in Section 5.6 that comprises a VEGF-D agent. In another specific embodiment, provided herein are methods for treating cancer, comprising administering to a subject in need thereof a first composition as described in Section 5.6 that comprises a oncolytic virus described herein, such as an APMV described herein, a second composition as described in Section 5.6 that comprises a VEGF-C agent, and a third composition as described in Section 5.6 that comprises a VEGF-D agent.

An oncolytic virus described herein, such as an APMV described herein (e.g., a naturally occurring or recombinant APMV described herein), or a composition thereof may be administered locally or systemically to a subject. For example, an oncolytic virus, such as an APMV described herein (e.g., a naturally occurring or recombinant APMV described herein), or a composition thereof may be administered parenterally (e.g., intraperitoneally, intravenously, intra-arterially, intradermally, intramuscularly, or subcutaneously), intratumorally, intra-nodally, intrapleurally, intranasally, intracavitary, intracranially, orally, rectally, by inhalation, or topically to a subject. In a specific embodiment, an oncolytic virus, such as an APMV described herein (e.g., a naturally occurring or recombinant APMV described herein), or a composition thereof is administered intratumorally. Image-guidance may be used to administer an oncolytic virus, such as an APMV described herein (e.g., a naturally occurring or recombinant APMV described herein), or a composition thereof to the subject. In a specific embodiment, an oncolytic virus, such as an APMV described herein (e.g., a naturally occurring or recombinant APMV described herein), or a composition thereof is administered intravenously.

A VEGF-C agent or a composition thereof, or a VEGF-D agent or a composition thereof may be administered locally or systemically to a subject. For example, a VEGF-C agent or a composition thereof, or a VEGF-D agent or a composition thereof may be administered parenterally (e.g., intraperitoneally, intravenously, intra-arterially, intradermally, intramuscularly, or subcutaneously), intratumorally, intra-nodally, intrapleurally, intranasally, intracavitary, intracranially, orally, rectally, by inhalation, or topically to a subject. In a specific embodiment, a VEGF-C agent or a composition thereof, or a VEGF-D agent or a composition thereof is administered intratumorally. Image-guidance may be used to administer a VEGF-C agent or a composition thereof, or a VEGF-D agent or a composition thereof to the subject. In a specific embodiment, a VEGF-C agent or a composition thereof, or a VEGF-D agent or a composition thereof is administered intravenously. In another specific embodiment, a VEGF-C agent or a composition thereof, or a VEGF-D agent or a composition thereof is administered intradermally.

In certain embodiments, the methods described herein include the treatment of cancer for which no treatment is available. In some embodiments, an oncolytic virus, such as an APMV described herein (e.g., a naturally occurring or recombinant APMV described herein), or a composition thereof is administered to a subject to treat cancer as an alternative to other conventional therapies. Cancers that may be treated in accordance with the methods described herein include those in Section 5.7.4.

In certain embodiments, two, three or multiple oncolytic viruses described herein, such as APMVs (including one, two or more recombinant APMVs described herein) are administered to a subject to treat cancer. The multiple oncolytic virus administered may be the same or different.

In another aspect, provided herein is a method of treating cancer comprising administering to a subject in need thereof polyI:C and a VEGFR-3 activating agent described herein. In some embodiments, the polyI:C and VEGFR-3 activating agent are administered to the subject in the same composition. In other embodiments, the polyI:C and VEGFR-3 activating agent are administered in different compositions. The polyI:C or composition thereof and VEGFR-3 activating agent or composition thereof may be administered by any route known in the art or described herein. For example, the polyI:C or composition thereof may be administered to a subject subcutaneously, intravenously, intramuscularly or intratumorally. The VEGFR-3 activating agent or composition thereof may be administered to a subject subcutaneously, intravenously, intramuscularly or intratumorally. In some embodiments, the methods of treating cancer do not comprise the administration of an antigen (e.g., a cancer antigen, a bacterial antigen, a fungal antigen, a protozoal antigen, a viral antigen or a helminth antigen) to the subject. In other embodiments, the methods of treating cancer do comprise the administration of an antigen (e.g., a cancer antigen, a bacterial antigen, a fungal antigen, a protozoal antigen, a viral antigen or a helminth antigen) to the subject. In certain embodiments, the polyI:C or composition thereof and VEGFR-3 activating agent or composition thereof are the only active agents administered to a subject in accordance with the methods described herein. In other embodiments, polyI:C or composition thereof and VEGFR-3 activating agent or composition thereof are administered to a subject in combination with another therapy described herein (see, e.g., section 5.7.2). Cancers that may be treated in accordance with the methods described herein are described herein (see, e.g., Section 5.7.4).

In a specific embodiment, a method of treating cancer described herein may result in a beneficial effect for a subject, such as the reduction, decrease, attenuation, diminishment, stabilization, remission, suppression, inhibition or arrest of the development or progression of cancer, or a symptom thereof. In certain embodiments, a method of treating cancer described herein results in at least one, two or more of the following effects: (i) the reduction or amelioration of the severity of cancer and/or a symptom associated therewith; (ii) the reduction in the duration of a symptom associated with cancer; (iii) the prevention in the recurrence of a symptom associated with cancer; (iv) the regression of cancer and/or a symptom associated therewith; (v) the reduction in hospitalization of a subject; (vi) the reduction in hospitalization length; (vii) the increase in the survival of a subject; (viii) the inhibition of the progression of cancer and/or a symptom associated therewith; (ix) the enhancement or improvement of the therapeutic effect of another therapy; (x) a reduction or elimination in the cancer cell population; (xi) a reduction in the growth of a tumor or neoplasm; (xii) a decrease in tumor size; (xiii) a reduction in the formation of a tumor; (xiv) eradication, removal, or control of primary, regional and/or metastatic cancer; (xv) a decrease in the number or size of metastases; (xvi) a reduction in mortality; (xvii) an increase in cancer-free survival rate of patients; (xviii) an increase in relapse-free survival; (xix) an increase in the number of patients in remission; (xx) a decrease in hospitalization rate; (xxi) the size of the tumor is maintained and does not increase in size or increases the size of the tumor by less than 5% or 10% after administration of a therapy as measured by conventional methods available to one of skill in the art, such as MRI, X-ray, CT Scan and PET scan; (xxii) the prevention of the development or onset of cancer and/or a symptom associated therewith; (xxiii) an increase in the length of remission in patients; (xxiv) the reduction in the number of symptoms associated with cancer; (xxv) an increase in symptom-free survival of cancer patients; (xxvi) limitation of or reduction in metastasis; (xxvii) overall survival; (xxviii) progression-free survival (as assessed, e.g., by RECIST v1.1.); (xxix) overall response rate; and/or (xxx) an increase in response duration. In some embodiments, the treatment/therapy that a subject receives does not cure cancer, but prevents the progression or worsening of the disease. In certain embodiments, a method of treating cancer described herein does not prevent the onset/development of cancer, but may prevent the onset of cancer symptoms. Any method known to the skilled artisan may be utilized to evaluate the treatment/therapy that a subject receives. In a specific embodiment, the efficacy of a treatment/therapy is evaluated according to the Response Evaluation Criteria In Solid Tumors (“RECIST”) published rules. In a specific embodiment, the efficacy of a treatment/therapy is evaluated according to the RECIST rules published in February 2000 (also referred to as “RECIST 1”) (see, e.g., Therasse et al., 2000, Journal of National Cancer Institute, 92(3):205-216, which is incorporated by reference herein in its entirety). In a specific embodiment, the efficacy of a treatment/therapy is evaluated according to the RECIST rules published in January 2009 (also referred to as “RECIST 1.1”) (see, e.g., Eisenhauer et al., 2009, European Journal of Cancer, 45:228-247, which is incorporated by reference herein in its entirety). In a specific embodiment, the efficacy of a treatment/therapy is evaluated according to the RECIST rules utilized by the skilled artisan at the time of the evaluation. In a specific embodiment, the efficacy is evaluated according to the immune related RECIST (“irRECIST”) published rules (see, e.g., Bohnsack et al., 2014, ESMO Abstract 4958, which is incorporated by reference herein in its entirety). In a specific embodiment, the efficacy treatment/therapy is evaluated according to the irRECIST rules utilized by the skilled artisan at the time of the evaluation. In a specific embodiment, the efficacy is evaluated through a reduction in tumor-associated serum markers.

In some embodiments, a method for treating cancer described herein increases infiltration of one, two or all of the following cell types into a tumor: (i) T-cells, (ii) natural killer (NK) cells, and (iii) dendritic cells. In certain embodiments, a method for treating cancer described herein increases lymphocyte infiltration into a tumor. In a specific embodiments, a method for treating cancer described herein increases T cell infiltration into a tumor. In certain embodiments, a method for treating cancer described herein increases CD4+ T cell infiltration into a tumor. In some embodiments, a method for treating cancer described herein increases CD8+ T cell infiltration into a tumor. In certain embodiments, a method for treating cancer described herein increases cytokine production in a tumor (e.g., increases INFγ, IL-2, and/or TNF production). In certain embodiments, a method for treating cancer described herein increases lymphatic drainage in a tumor. In some embodiments, a method for treating cancer described herein enhances an anti-tumor cell response (e.g., an anti-tumor T-cell response, anti-tumor NKcell response, and/or an anti-tumor dendritic cell response). In a specific embodiment, a method for treating cancer described herein enhances an anti-tumor T cell response.

In specific embodiments, a method for treating cancer described herein increases CD8, CD4 and NK cells within a tumor, such as, e.g., described in Example 5, infra. In certain embodiments, a method for treating cancer described herein increases one, two, three or all of the following within a tumor: (i) CD4-CD8− T-cells expressing TNF-α, (ii) CD4+ T cells expressing high levels of TNF-α and IFN-γ, (iii) CD8+ T-cells expressing TNF-α, IFN-γ, and GranzymeB, and (v) NK cells expressing Granzyme B, high levels of TNF-α and dim levels of IFN-γ. In some embodiments, a method for treating cancer described herein results in an increase in CD4+ and CD8+ T cells expressing CD83 and/or CD86 in sentinel lymph nodes, such as, e.g., described in Example 5, infra. In certain embodiments, a method for treating cancer described herein increases in sentinel lymph nodes CD83+ CD4 T cells, and tumor-specific CD103+CD83+ CD86+ CD8 T cells and CD83+ CD86+Ly6c+ CD8 T cells, such as, e.g., described in Example 5, infra. In some embodiments, a method for treating cancer described herein results in the enrichment of CD8, CD4 and CD11c+ dendritic cells associated with tumor lymphatic vessels in treated tumors, such as described in Example 5, infra. In certain embodiments, a method for treating cancer described herein results in immune activation both regionally (in sentinel lymph nodes) and systemically (in contralateral lymph nodes).

### 5.7.1 Dosage and Frequency

The amount of an oncolytic virus, such as an APMV described herein (e.g., a naturally occurring or recombinant APMV described herein), or a composition thereof which will be effective in the treatment of cancer will depend on the nature of the cancer, the route of administration, the general health of the subject, etc. and should be decided according to the judgment of a medical practitioner. Standard clinical techniques, such as in vitro assays, may optionally be employed to help identify dosage ranges. However, suitable dosage ranges of an oncolytic virus, such as an APMV described herein (e.g., a naturally occurring or recombinant APMV described herein), for administration are generally about 10², 5×10², 10³, 5×10³, 10⁴, 5×10⁴, 10⁵, 5×10⁵, 10⁶, 5×10⁶, 10⁷, 5×10⁷, 10⁸, 5×10⁸, 1×10⁹, 5×10⁹, 1×10¹⁰, 5×10¹⁰, 1×10¹¹, 5×10¹¹ or 10¹² pfu, and most preferably about 10⁴ to about 10¹², 10⁶ to 10¹², 10⁸ to 10¹², 10⁹ to 10¹², 10⁹ to 10¹¹ pfu, 10⁶ to 10¹⁰, or 10⁶ to 10⁸, and can be administered to a subject once, twice, three, four or more times with intervals as often as needed. Dosage ranges of oncolysate vaccines for administration may include 0.001 mg, 0.005 mg, 0.01 mg, 0.05 mg. 0.1 mg. 0.5 mg, 1.0 mg, 2.0 mg. 3.0 mg, 4.0 mg, 5.0 mg, 10.0 mg, 0.001 mg to 10.0 mg, 0.01 mg to 1.0 mg, 0.1 mg to 1 mg, and 0.1 mg to 5.0 mg, and can be administered to a subject once, twice, three or more times with intervals as often as needed. Dosage ranges of whole cell vaccines for administration may include 10², 5×10², 10³, 5×10³, 10⁴, 5×10⁴, 10⁵, 5×10⁵, 10⁶, 5×10⁶, 10⁷, 5×10⁷, 10⁸, 5×10⁸, 1×10⁹, 5×10⁹, 1×10¹⁰, 5×10¹⁰, 1×10¹¹, 5×10¹¹ or 10¹² cells, and can be administered to a subject once, twice, three or more times with intervals as often as needed. In certain embodiments, a dosage(s) of an oncolytic virus, such as an APMV described herein, similar to a dosage(s) currently being used in clinical trials for NDV is administered to a subject.

In certain embodiments, an oncolytic virus, such as an APMV described herein (e.g., a naturally occurring or recombinant APMV described herein), or a composition thereof is administered to a subject as a single dose followed by a second dose 1 to 6 weeks, 1 to 5 weeks, 1 to 4 weeks, 1 to 3 weeks, 1 to 2 weeks later. In accordance with these embodiments, booster inoculations may be administered to the subject at 3 to 6 month or 6 to 12 month intervals following the second inoculation.

In certain embodiments, an oncolytic virus, such as an APMV described herein (e.g., a naturally occurring or recombinant APMV described herein), or composition thereof is administered to a subject in combination with a VEGF-C agent or a VEGF-D agent. The dosage of the VEGF-C agent or VEGF-D agent will depend upon various factors including, e.g., the therapy, the nature of the cancer, the route of administration, the general health of the subject, etc. and should be decided according to the judgment of a medical practitioner. In specific embodiments, the dose of the VEGF-C agent or VEGF-D agent is 1 mg/kg to 100 mg/kg if the agent is proteinaceous. In certain embodiments, the single dose of the VEGF-C agent or VEGF-D agent is 1 μg to 200 μg if the agent is a nucleotide sequence. In certain embodiments, a therapeutically effective dose is administered. In specific embodiments, a therapeutically effective dose of the VEGF-C agent or VEGF-D agent is 1 mg/kg to 100 mg/kg if the agent is proteinaceous. In specific embodiments, a therapeutically effective dose of the VEGF-C agent or VEGF-D agent is 1 μg to 200 μg if the agent is a nucleotide sequence.

In certain embodiments, an oncolytic virus described herein, such as an APMV described herein (e.g., a naturally occurring or recombinant APMV described herein), or composition thereof is administered to a subject concurrently with the administration of a VEGF-C agent or VEGF-D agent. In other embodiments, an oncolytic virus described herein, such as an APMV described (e.g., a naturally occurring or recombinant APMV described herein), or composition thereof is administered to a subject every 3 to 7 days, 1 to 6 weeks, 1 to 5 weeks, 1 to 4 weeks, 2 to 4 weeks, 1 to 3 weeks, or 1 to 2 weeks and a VEGF-C agent or a VEGF-D agent is administered every 3 to 7 days, 1 to 6 weeks, 1 to 5 weeks, 1 to 4 weeks, 1 to 3 weeks, or 1 to 2 weeks. In certain embodiments, an oncolytic virus described herein, such as an APMV described (e.g., a naturally occurring or recombinant APMV described herein), or composition thereof is administered to a subject every day. In certain embodiments, an oncolytic virus described herein, such as an APMV described (e.g., a naturally occurring or recombinant APMV described herein), or composition thereof is administered to a subject every other day.

In certain embodiments, an oncolytic virus described herein, such as an APMV described herein (e.g., a naturally occurring or recombinant APMV described herein), or composition thereof is administered to a subject before or after the administration of a VEGF-C agent or VEGF-D agent. In other embodiments, an oncolytic virus described herein, such as an APMV described (e.g., a naturally occurring or recombinant APMV described herein), or composition thereof is administered to a subject 1 to 3 weeks, 2 weeks, 1 month, 2 months, or 3 months before or after a VEGF-C agent or a VEGF-D agent is administered. In other embodiments, an oncolytic virus described herein, such as an APMV described (e.g., a naturally occurring or recombinant APMV described herein), or composition thereof is administered to a subject 1, 2, 3, 4, 5, or 6 days before or after a VEGF-C agent or a VEGF-D agent is administered. In some embodiments, no additional therapies are administered to a subject (e.g., human subject) during the timeframe that the subject is receiving a VEGF-C agent or a VEGF-D agent and an oncolytic virus described herein, such as an APMV described (e.g., a naturally occurring or recombinant APMV described herein), or composition thereof. In other embodiments, one or more additional therapies, such as a therapy described in Section 5.7.2, infra, are administered to a subject (e.g., human subject) during the timeframe that the subject is receiving a VEGF-C agent or a VEGF-D agent and an oncolytic virus described herein, such as an APMV described (e.g., a naturally occurring or recombinant APMV described herein), or composition thereof.

In certain embodiments, an oncolytic virus, such as an APMV described herein (e.g., a naturally occurring or recombinant APMV described herein), or composition thereof is administered to a subject in combination with one or more additional therapies, such as a therapy described in Section 5.7.2, infra. The dosage of the other one or more additional therapies will depend upon various factors including, e.g., the therapy, the nature of the cancer, the route of administration, the general health of the subject, etc. and should be decided according to the judgment of a medical practitioner. In specific embodiments, the dose of the other therapy is the dose and/or frequency of administration of the therapy recommended for the therapy for use as a single agent is used in accordance with the methods disclosed herein. In other embodiments, the dose of the other therapy is a lower dose and/or involves less frequent administration of the therapy than recommended for the therapy for use as a single agent is used in accordance with the methods disclosed herein. Recommended doses for approved therapies can be found in the Physicians' Desk Reference (e.g., the 71^{st} ed. of the Physicians' Desk Reference (2017)).

In certain embodiments, an oncolytic virus described herein, such as an APMV described herein (e.g., a naturally occurring or recombinant APMV described herein), or composition thereof is administered to a subject concurrently with the administration of one or more additional therapies. In other embodiments, an oncolytic virus described herein, such as an APMV described (e.g., a naturally occurring or recombinant APMV described herein), or composition thereof is administered to a subject every 3 to 7 days, 1 to 6 weeks, 1 to 5 weeks, 1 to 4 weeks, 2 to 4 weeks, 1 to 3 weeks, or 1 to 2 weeks and one or more additional therapies (such as described in Section 5.6.2, infra) is administered every 3 to 7 days, 1 to 6 weeks, 1 to 5 weeks, 1 to 4 weeks, 1 to 3 weeks, or 1 to 2 weeks.

### 5.7.2 Additional Therapies

Additional therapies that can be used in a combination with an oncolytic virus described herein, such as an APMV described herein (e.g., a naturally occurring or recombinant APMV described herein), or a composition thereof for the treatment of cancer include, but are not limited to, small molecules, synthetic drugs, peptides (including cyclic peptides), polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules. In a specific embodiment, the additional therapy is a chemotherapeutic agent. In a specific embodiment, an additional therapy described herein may be used in combination with an oncolysate or whole cell vaccine described herein.

In some embodiments, an oncolytic virus described herein, such as an APMV described herein (e.g., a naturally occurring or recombinant APMV described herein) or a composition thereof is used in combination with radiation therapy comprising the use of x-rays, gamma rays and other sources of radiation to destroy cancer cells. In specific embodiments, the radiation therapy is administered as external beam radiation or teletherapy, wherein the radiation is directed from a remote source. In other embodiments, the radiation therapy is administered as internal therapy or brachytherapy wherein a radioactive source is placed inside the body close to cancer cells and/or a tumor mass.

Specific examples of anti-cancer agents that may be used in combination with an oncolytic virus described herein, such as an APMV described herein, or a composition thereof include: hormonal agents (e.g., aromatase inhibitor, selective estrogen receptor modulator (SERM), and estrogen receptor antagonist), chemotherapeutic agents (e.g., microtubule disassembly blocker, antimetabolite, topoisomerase inhibitor, and DNA crosslinker or damaging agent), radiation therapy, and conventional surgery.

In particular embodiments, an oncolytic virus described herein, such as an APMV described herein (e.g., a naturally occurring or recombinant APMV described herein), or a composition thereof is used in combination with an immunomodulatory agent. In a specific embodiment, an oncolytic virus described herein, such as an APMV described herein (e.g., a naturally occurring APMV or a recombinant APMV described herein), or composition thereof is used in combination with an agonist of a co-stimulatory receptor found on immune cells, such as, e.g., T-lymphocytes (e.g., CD4+ or CD8+ T-lymphocytes), NK cells and/or antigen-presenting cells (e.g., dendritic cells or macrophages), or a composition thereof. Specific examples of co-stimulatory receptors include glucocorticoid-induced tumor necrosis factor receptor (GITR), Inducible T-cell costimulator (ICOS or CD278), OX40 (CD134), CD27, CD28, 4-1BB (CD137), CD40, lymphotoxin alpha (LT alpha), LIGHT (lymphotoxin-like, exhibits inducible expression, and competes with herpes simplex virus glycoprotein D for HVEM, a receptor expressed by T lymphocytes), CD226, cytotoxic and regulatory T cell molecule (CRTAM), death receptor 3 (DR3), lymphotoxin-beta receptor (LTBR), transmembrane activator and CAML interactor (TACI), B cell-activating factor receptor (BAFFR), and B cell maturation protein (BCMA). In a specific embodiment, the agonist of the co-stimulatory molecule binds to a receptor on a cell (e.g., GITR, ICOS, OX40, CD70, 4-1BB, CD40, LIGHT, etc.) and triggers or enhances one or more signal transduction pathways. In a particular embodiment, the agonist of the co-stimulatory receptor is an antibody or ligand that binds to the co-stimulatory receptor and induces or enhances one or more signal transduction pathways. In certain embodiments, the agonist facilitates the interaction between a co-stimulatory receptor and its ligand(s). In certain embodiments, the agonist of a co-stimulatory receptor is an antibody (e.g., monoclonal antibody) that binds to glucocorticoid-induced tumor necrosis factor receptor (GITR), Inducible T-cell costimulator (ICOS or CD278), OX40 (CD134), CD27, CD28, 4-1BB (CD137), CD40, lymphotoxin alpha (LT alpha), LIGHT (lymphotoxin-like, exhibits inducible expression, and competes with herpes simplex virus glycoprotein D for HVEM, a receptor expressed by T lymphocytes), CD226, cytotoxic and regulatory T cell molecule (CRTAM), death receptor 3 (DR3), lymphotoxin-beta receptor (LTBR), transmembrane activator and CAML interactor (TACI), B cell-activating factor receptor (BAFFR), or B cell maturation protein (BCMA). In a specific embodiment, the agonist of a co-stimulatory receptor is an antibody (e.g., monoclonal antibody) that binds to 4-1BB or OX40.

In a specific embodiment, an oncolytic virus described herein, such as an APMV described herein (e.g., a naturally occurring or recombinant APMV described herein), or a composition thereof is used in combination with an antagonist of an inhibitory receptor found on immune cells, such as, e.g., T-lymphocytes (e.g., CD4+ or CD8+ T-lymphocytes), NK cells and/or antigen-presenting cells (e.g., dendritic cells or macrophages), or a composition thereof. Specific examples of inhibitory receptors include cytotoxic T-lymphocyte-associated antigen 4 (CTLA-4 or CD52), programmed cell death protein 1 (PD-1 or CD279), B and T-lymphocyte attenuator (BTLA), killer cell immunoglobulin-like receptor (KIR), lymphocyte activation gene 3 (LAG3), T-cell membrane protein 3 (TIM3), CD160, adenosine A2a receptor (A2aR), T cell immunoreceptor with immunoglobulin and ITIM domains (TIGIT), leukocyte-associated immunoglobulin-like receptor 1 (LAIR1), and CD160. In a specific embodiment, the antagonist inhibits the action of the inhibitory receptor without provoking a biological response itself. In a specific embodiment, the antagonist is an antibody or ligand that binds to an inhibitor receptor on an immune cell and blocks or dampens binding of the receptor to one or more of its ligands. In a particular embodiment, the antagonist of an inhibitory receptor is an antibody or a soluble receptor that specifically binds to the ligand for the inhibitory receptor and blocks the ligand from binding to the inhibitory receptor and transducing an inhibitory signal(s). Specific examples of ligands for inhibitory receptors include PD-L1, PD-L2, B7-H3, B7-H4, HVEM, Gal9 and adenosine. Specific examples of inhibitory receptors include CTLA-4, PD-1, BTLA, KIR, LAG3, TIM3, and A2aR.

In specific embodiments, the antagonist of an inhibitory receptor is a soluble receptor that specifically binds to a ligand for the inhibitory receptor and blocks the ligand from binding to the inhibitory receptor and transducing an inhibitory signal(s). In certain embodiments, the soluble receptor is a fragment of an inhibitory receptor (e.g., the extracellular domain of an inhibitory receptor). In some embodiments, the soluble receptor is a fusion protein comprising at least a portion of the inhibitory receptor (e.g., the extracellular domain of the native inhibitory receptor), and a heterologous amino acid sequence. In specific embodiments, the fusion protein comprises at least a portion of the inhibitory receptor, and the Fc portion of an immunoglobulin or a fragment thereof. In a specific embodiment, the antagonist of an inhibitory receptor is a LAG3-Ig fusion protein (e.g., IMP321).

In another embodiment, the antagonist of an inhibitory receptor is an antibody that specifically binds to a ligand(s) of the inhibitory receptor and blocks the ligand(s) from binding to the inhibitory receptor and transducing an inhibitory signal(s). Specific examples of ligands for inhibitory receptors include PD-L1, PD-L2, B7-H3, B7-H4, HVEM, Gal9 and adenosine. Specific examples of inhibitory receptors include CTLA-4, PD-1, BTLA, KIR, LAG3, and A2aR. In a specific embodiment, the antagonist is an antibody that binds to PD-L1 or PD-L2.

In another embodiment, the antagonist of an inhibitory receptor is an antibody that binds to the inhibitory receptor and blocks the binding of the inhibitory receptor to one, two or more of its ligands. In a specific embodiment, the binding of the antibody to the inhibitory receptor does not transduce an inhibitory signal(s) or blocks an inhibitory signal(s). Specific examples of inhibitory receptors include CTLA-4, PD-1, BTLA, KIR, LAG3, TIM3, and A2aR. A specific example of an antibody to inhibitory receptor is anti-CTLA-4 antibody (Leach D R, et al. Science 1996; 271: 1734-1736). In a specific embodiment, an antagonist of an inhibitory receptor is an antagonist of CTLA-4, such as, e.g., Ipilimumab or Tremelimumab.

In certain embodiments, the antagonist of an inhibitory receptor is an antagonist of PD-1, such as, e.g., Nivolumab (MDX-1106 or BMS-936558), pembrolizumab (MK3475), pidlizumab (CT-011), AMP-224 (a PD-L2 fusion protein), Atezoliuzumab (MPDL3280A; anti-PD-L1 monoclonal antibody), Avelumab (an anti-PD-L1 monoclonal antibody) or MDX-1105 (an anti-PD-L1 monoclonal antibody). In certain embodiments, an antagonist of an inhibitory receptor is an antagonist of LAG3, such as, e.g., IMP321.

In a specific embodiment, an antagonist of an inhibitory receptor is an anti-PD-1 antibody that blocks the interaction between PD-1 and its ligands (PD-L1 and PD-L2). Non-limiting examples of antibodies that bind to PD-1 include pembrolizumab (“KEYTRUDA®”; see, e.g., Hamid et al., N Engl J Med. 2013; 369:134-44 and Full Prescribing Information for KEYTRUDA, Reference ID: 3862712), nivolumab (“OPDIVO®”; see, e.g., Topalian et al., N Engl J Med. 2012; 366:2443-54 and Full Prescribing Information for OPDIVO (nivolumab), Reference ID: 3677021), and MEDI0680 (also referred to as “AMP-514”; see, e.g., Hamid et al., Ann Oncol. 2016; 27(suppl_6):1050PD). In a specific embodiment, the antagonist of an inhibitory receptor is an anti-PD1 antibody (e.g., pembrolizumab).

In a specific embodiment, an oncolytic virus described herein, such as an APMV described herein (e.g., a naturally occurring or recombinant APMV described herein), or a composition thereof is used in combination with a checkpoint inhibitor. In a specific embodiment, the checkpoint inhibitor may be an antibody that binds to an inhibitory receptor found on a T cell, such as PD-1, CTLA-4, LAG-3, or TIM-3. In another specific embodiment, the checkpoint inhibitor may be an antibody that binds to an inhibitory receptor found on a T cell, such as PD-1, CTLA-4, LAG-3, or TIM-3 and blocks binding of the inhibitory receptor to its ligand(s).

In a specific embodiment, an oncolytic virus described herein, such as an APMV described herein (e.g., a naturally occurring or recombinant APMV described herein), or a composition thereof is used in combination with an anti-PD1 antibody that blocks binding of PD1 to its ligand(s) (e.g., either PD-L1, PD-L2, or both), such as described herein or known to one of skill in the art, or a composition thereof. In a specific embodiment, the antibody is a monoclonal antibody.

In a specific embodiment, an oncolytic virus described herein, such as an APMV described herein (e.g., a naturally occurring or recombinant APMV described herein), or a composition thereof is used in combination with an anti-PD-L1 antibody (e.g., an anti-PD-L1 antibody described herein or known to one of skill in art), or a composition thereof. In a specific embodiment, the antibody is a monoclonal antibody.

In a specific embodiment, an oncolytic virus described herein, such as an APMV described herein (e.g., a naturally occurring or recombinant APMV described herein), or a composition thereof is used in combination with an anti-PD-L2 antibody (e.g., an anti-PD-L2 antibody described herein or known to one of skill in art), or a composition thereof. In a specific embodiment, the antibody is a monoclonal antibody. In a specific embodiment, an oncolytic virus described herein, such as an APMV described herein (e.g., a naturally occurring or recombinant APMV described herein), or a composition thereof is used in combination with a RIG-1 agonist (e.g., poly-dA-dT (otherwise known as poly(deoxyadenylic-deoxythymidylic) acid sodium salt)), or a composition thereof. In another specific embodiment, an oncolytic virus described herein, such as an APMV described herein (e.g., a naturally occurring or recombinant APMV described herein), or a composition thereof is used in combination with an MDA-5 agonist or a composition thereof. In another specific embodiment, an oncolytic virus described herein, such as an APMV described herein (e.g., a naturally occurring or recombinant APMV described herein), or a composition thereof is used in combination with a NOD1/NOD2 agonist (e.g., MurNAc-L-Ala-γ-D-Glu-mDAP) or a composition thereof. In another specific embodiment, an oncolytic virus described herein, such as an APMV described herein (e.g., a naturally occurring or recombinant APMV described herein), or a composition thereof is used in combination with an agent that activates cGAS/STING signalling (e.g., cGAMP, such as 2′3′ cGAMP) or a composition thereof. In another specific embodiment, an oncolytic virus described herein, such as an APMV described herein (e.g., a naturally occurring or recombinant APMV described herein), or a composition thereof is used in combination with Toll-like receptor agonist (e.g., BCG, PolyI:C, Poly ICLC, MPL, Imiquimod, CpG ODN (see, e.g., Braunstein et al., 2018, Target Oncol. 13(5):583-598 for examples of such agents)) or a composition thereof. In another specific embodiment, an oncolytic virus described herein, such as an APMV described herein (e.g., a naturally occurring or recombinant APMV described herein), or a composition thereof is used in combination with an antibody that specifically binds to CD3 or a composition thereof.

Currently available cancer therapies and their dosages, routes of administration and recommended usage are known in the art and have been described in such literature as the Physicians' Desk Reference (71st ed., 2017).

### 5.7.3 Patient Population

In some embodiments, an oncolytic virus described herein, such as an APMV described herein (e.g., a naturally occurring or recombinant APMV described herein), or a composition thereof, or a combination therapy described herein is administered to a subject suffering from cancer. In other embodiments, an oncolytic virus described herein, such as an APMV described herein (e.g., a naturally occurring or recombinant APMV described herein), or a composition thereof, or a combination therapy described herein is administered to a subject predisposed or susceptible to cancer. In some embodiments, an oncolytic virus described herein, such as an APMV described herein (e.g., a naturally occurring or recombinant APMV described herein) or a composition thereof, or a combination therapy described herein is administered to a subject diagnosed with cancer. Specific examples of the types of cancer are described herein (see, e.g., Section 5.6.4 and Section 6). In an embodiment, the subject has metastatic cancer. In another embodiment, the subject has stage 1, stage 2, stage 3, or stage 4 cancer. In another embodiment, the subject is in remission. In yet another embodiment, the subject has a recurrence of cancer.

In certain embodiments, an oncolytic virus described herein, such as an APMV described herein (e.g., a naturally occurring or recombinant APMV described herein), or a composition thereof, or a combination therapy described herein is administered to a human that is 0 to 6 months old, 6 to 12 months old, 6 to 18 months old, 18 to 36 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old. In some embodiments, an oncolytic virus described herein, such as an APMV described herein (e.g., a naturally occurring or recombinant APMV described herein), or a composition thereof, or a combination therapy described herein is administered to a human infant. In other embodiments, an oncolytic virus described herein, such as an APMV described herein (e.g., a naturally occurring or recombinant APMV described herein), or a composition thereof, or a combination therapy described herein is administered to a human toddler. In other embodiments, an oncolytic virus described herein, such as an APMV described herein (e.g. a naturally occurring or recombinant APMV described herein), or a composition thereof, or a combination therapy described herein is administered to a human child. In other embodiments, an oncolytic virus described herein, such as an APMV described herein (e.g., a naturally occurring or recombinant APMV described herein)m or a composition thereof, or a combination therapy described herein is administered to a human adult. In yet other embodiments, an oncolytic virus described herein, such as an APMV described herein (e.g., a naturally occurring or recombinant APMV described herein), or a composition thereof, or a combination therapy described herein is administered to an elderly human.

In certain embodiments, an oncolytic virus described herein, such as an APMV described herein (e.g., a naturally occurring or recombinant APMV described herein), or a composition thereof, or a combination therapy described herein is administered to a subject in an immunocompromised state or immunosuppressed state or at risk for becoming immunocompromised or immunosuppressed. In certain embodiments, an oncolytic virus described herein, such as an APMV described herein (e.g., a naturally occurring or recombinant APMV described herein), or a composition thereof, or a combination therapy described herein is administered to a subject receiving or recovering from immunosuppressive therapy. In certain embodiments, an oncolytic virus described herein, such as an APMV described herein (e.g., a naturally occurring or recombinant APMV described herein), or a composition thereof, or a combination therapy described herein is administered to a subject that has or is at risk of getting cancer. In certain embodiments, the subject is, will or has undergone surgery, chemotherapy and/or radiation therapy. In certain embodiments, the patient has undergone surgery to remove the tumor or neoplasm. In specific embodiments, the patient is administered an oncolytic virus described herein, such as an APMV described herein (e.g., a naturally occurring or recombinant APMV described herein), or a composition thereof, or a combination therapy described herein following surgery to remove a tumor or neoplasm. In other embodiments, the patient is administered an oncolytic virus described herein, such as an APMV described herein (e.g., a naturally occurring or recombinant APMV described herein), or a composition thereof, or a combination therapy described herein prior to undergoing surgery to remove a tumor or neoplasm. In certain embodiments, an oncolytic virus described herein, such as an APMV described herein (e.g., a naturally occurring or recombinant APMV described herein), or a composition thereof, or a combination therapy described herein is administered to a subject that has, will have or had a tissue transplant, organ transplant or transfusion.

In some embodiments, an oncolytic virus described herein, such as an APMV described herein (e.g., a naturally occurring or recombinant APMV described herein), or a composition thereof, or a combination therapy described herein is administered to a patient who has proven refractory to therapies other than the oncolytic virus (e.g., APMV) or composition thereof, or a combination therapy but are no longer on these therapies. In a specific embodiment, an oncolytic virus described herein, such as an APMV described herein (e.g., a naturally occurring or recombinant APMV described herein), or a composition thereof, or a combination therapy described herein is administered to a patient who has proven refractory to chemotherapy. The determination of whether cancer is refractory can be made by any method known in the art. In a certain embodiment, refractory patient is a patient refractory to a standard therapy. In some embodiments, a patient with cancer is initially responsive to therapy, but subsequently becomes refractory.

In certain embodiments, a cancer treated in accordance with the methods described herein has low levels of or no detectable levels of VEGF-C expression, as assessed by determining the level of expression of VEGF-C in a tumor biopsy sample using techniques known to one of skill in the art, such as immunohistochemistry, ELISA, RNA-seq or qPCR. In some embodiments, a cancer treated in accordance with the methods described herein has moderate to high levels of VEGF-C expression, as assessed by determining the level of expression of VEGF-C by tumor biopsy sample using techniques known to one of skill in the art, such as immunohistochemistry, ELISA, RNA-seq or qPCR. Low, moderate or high levels of VEGF-C may be determined by comparison to a healthy donor control sample or can be expressed as relative to other cancers of the same type in the patient population. In a specific embodiment, a transcriptome from a patient is compared to transcriptomes from healthy tissue samples from healthy control subjects or to transcriptomes from tumor biopsies of patients with the same or similar type of cancer using, e.g., data from the Genotype-Tissue Expression project (GTEX), The Cancer Genome Atlas (TCGA), or both.

In certain embodiments, a cancer treated in accordance with the methods described herein has low levels of or no detectable levels of VEGF-D expression, as assessed by determining the level of expression of VEGF-D in a tumor biopsy sample using techniques known to one of skill in the art, such as immunohistochemistry, ELISA, RNA-seq or qPCR. In some embodiments, a cancer treated in accordance with the methods described herein has moderate to high levels of VEGF-D expression, as assessed by determining the level of expression of VEGF-D by tumor biopsy sample using techniques known to one of skill in the art, such as immunohistochemistry, ELISA, RNA-seq or qPCR. Low, moderate or high levels of VEGF-D may be determined by comparison to a healthy donor control sample or can be expressed as relative to other cancers of the same type in the patient population. In a specific embodiment, a transcriptome from a patient is compared to transcriptomes from healthy tissue samples from healthy control subjects or to transcriptomes from tumor biopsies of patients with the same or similar type of cancer using, e.g., data from the Genotype-Tissue Expression project (GTEX), The Cancer Genome Atlas (TCGA), or both.

### 5.7.4 Types of Cancers

Specific examples of cancers that can be treated in accordance with the methods described herein include, but are not limited to: melanomas, leukemias, lymphomas, multiple myelomas, sarcomas, and carcinomas. In one embodiment, cancer treated in accordance with the methods described herein is a leukemia, such as acute leukemia, acute lymphocytic leukemia, acute myelocytic leukemias, such as, myeloblastic, promyelocytic, myelomonocytic, monocytic, erythroid leukemias, and myelodysplastic syndrome. In another embodiment, cancer treated in accordance with the methods described herein is a chronic leukemia, such as chronic myelocytic (granulocytic) leukemia, chronic lymphocytic leukemia, and hairy cell leukemia. In another embodiment, cancer treated in accordance with the methods described herein is a lymphoma, such as Hodgkin disease and non-Hodgkin disease. In another embodiment, cancer treated in accordance with the methods described herein is a multiple myeloma such as smoldering multiple myeloma, nonsecretory myeloma, osteosclerotic myeloma, solitary plasmacytoma and extramedullary plasmacytoma. In another embodiment, cancer treated in accordance with the methods described herein is Waldenström's macroglobulinemia monoclonal gammopathy of undetermined significance, benign monoclonal gammopathy, Wilm's tumor, or heavy chain disease

In one embodiment, cancer treated in accordance with the methods described herein is bone cancer, brain cancer, breast cancer, adrenal cancer, thyroid cancer, pancreatic cancer, pituitary cancer, eye cancer, vaginal, vulvar cancer, cervical cancer, uterine cancer, ovarian cancer, esophageal cancer, stomach cancer, colon cancer, rectal cancer, liver cancer, gallbladder cancer, lung cancer, testicular cancer, prostate cancer, penal cancer, oral cancer, basal cancer, salivary gland cancer, pharynx cancer, skin cancer, kidney cancer, or bladder cancer. In another embodiment, cancer treated in accordance with the methods described herein is brain, breast, lung, colorectal, liver, kidney or skin cancer.

In another embodiment, cancer treated in accordance with the methods described herein is a bone and connective tissue sarcoma, such as bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, or synovial sarcoma. In another embodiment, cancer treated in accordance with the methods described herein is a brain tumor, such as glioma, astrocytoma, brain stem glioma, ependymoma, oligodendroglioma, nonglial tumor, glioblastoma multiforme, acoustic neurinoma, craniopharyngioma, medulloblastoma, meningioma, pineocytoma, pineoblastoma, or primary brain lymphoma. In another embodiment, cancer treated in the accordance with the methods described herein is breast cancer, such as triple negative breast cancer, ER+/HER2-breast cancer, ER+/PR+/HER2+ breast cancer, ER−/PR−/Her2− breast cancer, ductal carcinoma, adenocarcinoma, lobular (cancer cell) carcinoma, intraductal carcinoma, medullary breast cancer, mucinous breast cancer, tubular breast cancer, papillary breast cancer, Paget's disease, or inflammatory breast cancer. In another embodiment, cancer treated in the accordance with the methods described herein is adrenal cancer, such as pheochromocytom or adrenocortical carcinoma. In another embodiment, cancer treated in the accordance with the methods described herein is thyroid cancer, such as papillary or follicular thyroid cancer, medullary thyroid cancer or anaplastic thyroid cancer. In another embodiment, cancer treated in the accordance with the methods described herein is pancreatic cancer, such as insulinoma, gastrinoma, glucagonoma, vipoma, somatostatin-secreting tumor, or carcinoid or islet cell tumor. In another embodiment, cancer treated in the accordance with the methods described herein is pituitary cancer, such as Cushing's disease, prolactin-secreting tumor, acromegaly, or diabetes insipidus. In another embodiment, cancer treated in the accordance with the methods described herein is eye cancer, such as ocular melanoma such as iris melanoma, choroidal melanoma, ciliary body melanoma, or retinoblastoma. In another embodiment, cancer treated in the accordance with the methods described herein is vaginal cancer, such as squamous cell carcinoma, adenocarcinoma, or melanoma. In another embodiment, cancer treated in the accordance with the methods described herein is vulvar cancer, such as squamous cell carcinoma, melanoma, adenocarcinoma, basal cell carcinoma, sarcoma, or Paget's disease. In another embodiment, cancer treated in the accordance with the methods described herein is cervical cancer, such as squamous cell carcinoma or adenocarcinoma. In another embodiment, cancer treated in the accordance with the methods described herein is uterine cancer, such as endometrial carcinoma or uterine sarcoma.

In another embodiment, cancer treated in accordance with the methods described herein is ovarian cancer, such as ovarian epithelial carcinoma, borderline tumor, germ cell tumor, or stromal tumor. In another embodiment, cancer treated in accordance with the methods described herein is esophageal cancer, such as squamous cancer, adenocarcinoma, adenoid cystic carcinoma, mucoepidermoid carcinoma, adenosquamous carcinoma, sarcoma, melanoma, placancercytoma, verrucous carcinoma, or oat cell (cancer cell) carcinoma. In another embodiment, cancer treated in accordance with the methods described herein is stomach cancer, such as adenocarcinoma, fungating (polypoid), ulcerating, superficial spreading, diffusely spreading, malignant lymphoma, liposarcoma, fibrosarcoma, or carcinosarcoma. In another embodiment, cancer treated in accordance with the methods described herein is liver cancer, such as hepatocellular carcinoma or hepatoblastoma. In another embodiment, cancer treated in accordance with the methods described herein is gallbladder cancer, such as adenocarcinoma. In another embodiment, cancer treated in accordance with the methods described herein is cholangiocarcinoma, such as papillary, nodular, or diffuse. In another embodiment, cancer treated in accordance with the methods described herein is lung cancer, such as non-small cell lung cancer, squamous cell carcinoma (epidermoid carcinoma), adenocarcinoma, large-cell carcinoma or cancer-cell lung cancer. In another embodiment, cancer treated in accordance with the methods described herein is testicular cancer, such germinal tumor, seminoma, anaplastic, classic (typical), spermatocytic, nonseminoma, embryonal carcinoma, teratoma carcinoma, or choriocarcinoma (yolk-sac tumor). In another embodiment, cancer treated in accordance with the methods described herein is prostate cancer, such as prostatic intraepithelial neoplasia, adenocarcinoma, leiomyosarcoma, or rhabdomyosarcoma. In another embodiment, cancer treated in accordance with the methods described herein is penal cancers. In another embodiment, cancer treated in accordance with the methods described herein is oral cancer, such as squamous cell carcinoma. In another embodiment, cancer treated in accordance with the methods described herein is salivary gland cancer, such as adenocarcinoma, mucoepidermoid carcinoma, or adenoidcystic carcinoma. In another embodiment, cancer treated in accordance with the methods described herein is pharynx cancer, such as squamous cell cancer or verrucous. In another embodiment, cancer treated in accordance with the methods described herein is skin cancer, such as basal cell carcinoma, squamous cell carcinoma and melanoma, superficial spreading melanoma, nodular melanoma, lentigo malignant melanoma, or acral lentiginous melanoma. In another embodiment, cancer treated in accordance with the methods described herein is kidney cancer, such as renal cell carcinoma, adenocarcinoma, hypernephroma, fibrosarcoma, or transitional cell cancer (renal pelvis and/or uterine). In another embodiment, cancer treated in accordance with the methods described herein is bladder cancer, such as transitional cell carcinoma, squamous cell cancer, adenocarcinoma, or carcinosarcoma.

In a specific embodiment, the cancer treated in accordance with the methods described herein is a melanoma. In another specific embodiment, the cancer treated in accordance with the methods described herein is a lung carcinoma. In another specific embodiment, the cancer treated in accordance with the methods described herein is a colorectal carcinoma. In a specific embodiment, the cancer treated in accordance with the methods described herein is melanoma, non-small cell lung cancer, head and neck squamous cell cancer, classical Hodgkin lymphoma, primary mediastinal large B-cell lymphoma, urothelial carcinoma, microsatellite instability-high cancer, gastric cancer, or cervical cancer.

In a specific embodiment, an oncolytic virus described herein (e.g., an AMPV) or compositions thereof, or a combination therapy described herein are useful in the treatment of a variety of cancers and abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, teratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma.

In some embodiments, cancers associated with aberrations in apoptosis are treated in accordance with the methods described herein. Such cancers may include, but are not limited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders of the skin, lung, liver, bone, brain, stomach, colon, breast, prostate, bladder, kidney, pancreas, ovary, uterus or any combination of the foregoing are treated in accordance with the methods described herein. In other specific embodiments, a sarcoma or melanoma is treated in accordance with the methods described herein.

In a specific embodiment, the cancer being treated in accordance with the methods described herein is leukemia, lymphoma or myeloma (e.g., multiple myeloma). Specific examples of leukemias and other blood-borne cancers that can be treated in accordance with the methods described herein include, but are not limited to, acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CIVIL”, chronic lymphocytic leukemia “CLL”, and hairy cell leukemia.

Specific examples of lymphomas that can be treated in accordance with the methods described herein include, but are not limited to, Hodgkin disease, non-Hodgkin lymphoma such as diffuse large B-cell lymphoma, multiple myeloma, Waldenström's macroglobulinemia, heavy chain disease, and polycythemia vera.

In another embodiment, the cancer being treated in accordance with the methods described herein is a solid tumor. Examples of solid tumors that can be treated in accordance with the methods described herein include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, cancer cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma. In another embodiment, the cancer being treated in accordance with the methods described herein is a metastatic. In another embodiment, the cancer being treated in accordance with the methods described herein is malignant.

In a specific embodiment, the cancer being treated in accordance with the methods described herein is a cancer that has a poor prognosis and/or has a poor response to conventional therapies, such as chemotherapy and radiation. In another specific embodiment, the cancer being treated in accordance with the methods described herein is malignant melanoma, malignant glioma, renal cell carcinoma, pancreatic adenocarcinoma, malignant pleural mesothelioma, lung adenocarcinoma, lung small cell carcinoma, lung squamous cell carcinoma, anaplastic thyroid cancer, or head and neck squamous cell carcinoma. In another specific embodiment, the cancer being treated in accordance with the methods described herein is a type of cancer described in Section 6, infra.

In a specific embodiment, the cancer being treated in accordance with the methods described herein is a cancer that is metastatic. In a specific embodiment, the cancer comprises a dermal, subcutaneous, or nodal metastasis. In a specific embodiment, the cancer comprises peritoneal or pleural metastasis. In a specific embodiment, the cancer comprises visceral organ metastasis, such as liver, kidney, spleen, or lung metastasis.

In a specific embodiment, the cancer being treated in accordance with the methods described herein is a cancer that is unresectable. Any method known to the skilled artisan may be utilized to determine if a cancer is unresectable.

### 5.8 Biological Assays

In a specific embodiment, one, two or more of the assays described in Section 6 may be used to characterize an oncolytic virus described herein, such as an APMV described herein. In certain embodiments, the expression, the activity (e.g., one, two or more functions), or both of a VEGFR-3 activating agent is determined using techniques known to one of skill in the art. In certain embodiments, the expression, the activity (e.g., one, two or more functions), or both of a VEGF-C agent is determined using techniques known to one of skill in the art. In some embodiments, the expression, the activity (e.g., one, two or more functions), or both of a VEGF-D agent is determined using techniques known to one of skill in the art. For example, the expression of a VEGF-C or VEGF-D agent may be determined using a qPCR or an immunoassay, such as a Western Blot, an ELISA or immunohistochemistry. The ability of VEGF-C or VEGF-D agent to bind to VEGFR-3 and VEGFR-2, may be determined using techniques known in the art. The ability of a VEGFR-3 activating agent to induce phosphorylation of VEGFR-3 and downstream phosphorylation of serine/threonine kinases, such as, e.g., AKT, ERK1/2 and Stat3 may be determined using techniques known in the art, such as Western blotting or protein arrays. The ability of a VEGFR-3 activating agent to modulate proliferation of cells may be determined using techniques known in the art, such as growth assays or clonogenic survival assays. The ability of a VEGFR-3 activating agent to modulate migration of cells may be determined using techniques known in the art, such as transwell migration assays and scratch assays. The ability of a VEGFR-3 activating agent to modulate tube formation of lymphatic endothelial cells may be determined using techniques known in the art. See, e.g., Nowak-Sliwinska et al., 2018, Angiogenesis 21: 425-532; Oliver et al., Oliver G., Kahn M. (eds) Lymphangiogenesis. Methods in Molecular Biology, 2018, vol 1846. Humana Press, New York, N.Y.; and Gibot et al., 2016, Biomaterials 78:129-39 for examples of assays.

### 5.8.1 In Vitro Assays

Viral assays include those that indirectly measure viral replication (as determined, e.g., by plaque formation) or the production of viral proteins (as determined, e.g., by western blot analysis) or viral RNAs (as determined, e.g., by RT-PCR or northern blot analysis) in cultured cells in vitro using methods which are well known in the art.

Growth of an oncolytic virus described herein, such as an APMV described herein, can be assessed by any method known in the art or described herein (e.g., in cell culture (e.g., cultures of chicken embryonic kidney cells or cultures of chicken embryonic fibroblasts (CEF)) (see, e.g., Section 6). Viral titer may be determined by inoculating serial dilutions of a recombinant APMV described herein into cell cultures (e.g., CEF, MDCK, EFK-2 cells, Vero cells, primary human umbilical vein endothelial cells (HUVEC), H292 human epithelial cell line or HeLa cells), chick embryos, or live animals (e.g., avians). After incubation of the virus for a specified time, the virus is isolated using standard methods. Physical quantitation of the virus titer can be performed using PCR applied to viral supernatants (Quinn & Trevor, 1997; Morgan et al., 1990), hemagglutination assays, tissue culture infectious doses (TCID50) or egg infectious doses (EID50). An exemplary method of assessing viral titer is described in Section 6, below.

Incorporation of nucleotide sequences encoding a heterologous peptide or protein (e.g., a transgene into the genome of an oncolytic virus described herein, such as an APMV described herein, can be assessed by any method known in the art or described herein (e.g., in cell culture, an animal model or viral culture in embryonated eggs)). For example, viral particles from cell culture of the allantoic fluid of embryonated eggs can be purified by centrifugation through a sucrose cushion and subsequently analyzed for protein expression by Western blotting using methods well known in the art.

Immunofluorescence-based approaches may also be used to detect virus and assess viral growth. Such approaches are well known to those of skill in the art, e.g., fluorescence microscopy and flow cytometry (see, eg., Section 6, infra). Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, Oreg.; Sigma-Aldrich (2003) Catalogue, St. Louis, Mo.). See, e.g., the assays described in Section 6, infra.

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.). See also Section 6, infra, for histology and immunohistochemistry assays that may be used.

### 5.8.2 Interferon Assays

IFN induction by an oncolytic virus described herein, such as an APMV described herein, may be determined using techniques known to one of skill in the art. For example, the amount of IFN induced in cells following infection with an oncolytic virus described herein, such as an APMV described herein, may be determined using an immunoassay (e.g., an ELISA or Western blot assay) to measure IFN expression or to measure the expression of a protein whose expression is induced by IFN. Alternatively, the amount of IFN induced may be measured at the RNA level by assays, such as Northern blots and quantitative RT-PCR, known to one of skill in the art. In specific embodiments, the amount of IFN released may be measured using an ELISPOT assay. Further, the induction and release of cytokines and/or interferon-stimulated genes may be determined by, e.g., an immunoassay or ELISPOT assay at the protein level and/or quantitative RT-PCR or northern blots at the RNA level.

### 5.8.3 Activation Marker Assays and Immune Cell Infiltration Assay

The expression of a T cell marker, B cell marker, activation marker, co-stimulatory molecule, ligand, or inhibitory molecule by immune cells induced by an oncolytic virus described herein, such as an APMV described herein, may be assessed. Techniques for assessing the expression of T cell marker, B cell marker, activation marker, co-stimulatory molecule, ligand, or inhibitory molecule by immune cells are known to one of skill in the art. For example, the expression of T cell marker, B cell marker, an activation marker, co-stimulatory molecule, ligand, or inhibitory molecule by an immune cell can be assessed by flow cytometry. In a specific embodiment, a method described in Section 6, infra, is used to assess immune cell infiltration, activation or both.

### 5.8.4 Toxicity Studies

In some embodiments, an oncolytic virus described herein, such as an APMV described herein, or composition thereof, or a combination therapy described herein are tested for cytotoxicity in mammalian, preferably human, cell lines. In certain embodiments, cytotoxicity is assessed in one or more of the following non-limiting examples of cell lines: U937, a human monocyte cell line; primary peripheral blood mononuclear cells (PBMC); Huh7, a human hepatoblastoma cell line; HL60 cells, HT1080, HEK 293T and 293H, MLPC cells, human embryonic kidney cell lines; human melanoma cell lines, such as SkMe12, SkMe1-119 and SkMe1-197; THP-1, monocytic cells; a HeLa cell line; and neuroblastoma cells lines, such as MC-IXC, SK-N-MC, SK-N-MC, SK-N-DZ, SH-SY5Y, and BE(2)-C. In some embodiments, the ToxLite assay is used to assess cytotoxicity.

Many assays well-known in the art can be used to assess viability of cells or cell lines following infection with an oncolytic virus described herein, such as an APMV described herein, or composition thereof, and, thus, determine the cytotoxicity of the APMV or composition thereof. For example, cell proliferation can be assayed by measuring Bromodeoxyuridine (BrdU) incorporation, (³H) thymidine incorporation, by direct cell count, or by detecting changes in transcription, translation or activity of known genes such as proto-oncogenes (e.g., fos, myc) or cell cycle markers (Rb, cdc2, cyclin A, D1, D2, D3, E, etc.). The levels of such protein and mRNA and activity can be determined by any method well known in the art. For example, protein can be quantitated by known immunodiagnostic methods such as ELISA, Western blotting or immunoprecipitation using antibodies, including commercially available antibodies. mRNA can be quantitated using methods that are well known and routine in the art, for example, using northern analysis, RNase protection, or polymerase chain reaction in connection with reverse transcription. Cell viability can be assessed by using trypan-blue staining or other cell death or viability markers known in the art. In a specific embodiment, the level of cellular ATP is measured to determined cell viability. In preferred embodiments, an APMV described herein or composition thereof does not kill healthy (i.e., non-cancerous) cells.

In specific embodiments, cell viability may be measured in three-day and seven-day periods using an assay standard in the art, such as the CellTiter-Glo Assay Kit (Promega) which measures levels of intracellular ATP. A reduction in cellular ATP is indicative of a cytotoxic effect. In another specific embodiment, cell viability can be measured in the neutral red uptake assay. In other embodiments, visual observation for morphological changes may include enlargement, granularity, cells with ragged edges, a filmy appearance, rounding, detachment from the surface of the well, or other changes.

The oncolytic viruses described herein, such as an APMVs described herein, or compositions thereof, or combination therapies can be tested for in vivo toxicity in animal models. For example, animal models, known in the art to test the effects of compounds on cancer can also be used to determine the in vivo toxicity of an oncolytic virus described herein, such as an APMV described herein, or a composition thereof, or combination therapies. For example, animals are administered a range of pfu of an oncolytic virus described herein, such as an APMV described herein, and subsequently, the animals are monitored over time for various parameters, such as one, two or more of the following: lethality, weight loss or failure to gain weight, and levels of serum markers that may be indicative of tissue damage (e.g., creatine phosphokinase level as an indicator of general tissue damage, level of glutamic oxalic acid transaminase or pyruvic acid transaminase as indicators for possible liver damage). These in vivo assays may also be adapted to test the toxicity of various administration mode and regimen in addition to dosages.

The toxicity, efficacy or both of an oncolytic virus described herein, such as an APMV described herein, or a composition thereof, or a combination therapy described herein can be determined by standard pharmaceutical procedures in cell cultures or experimental animals. In a specific embodiment, the cytotoxicity of an oncolytic virus described herein, such as an APMV described herein, is determined by methods set forth in Section 6, infra.

The data obtained from the cell culture assays and animal studies can be used in formulating a range of dosage of the therapies for use in subjects.

### 5.8.5 Biological Activity Assays

An oncolytic virus described herein, such as an APMV described herein, or a composition thereof, or a combination therapy described herein can be tested for biological activity using animal models for treating cancer. (see, e.g., Section 6). For example, an oncolytic virus described herein, such as an APMV described herein, or a composition thereof, and a VEGFR-3-activating agent described herein can be tested for biological activity using animal models for treating cancer. In another example, an oncolytic virus described herein, such as an APMV described herein, or a composition thereof, and a VEGF-C agent or a VEGF-D agent described herein can be tested for biological activity using animal models for treating cancer. Such animal model systems include, but are not limited to, rats, mice, hamsters, cotton rats, chicken, cows, monkeys (e.g., African green monkey), pigs, dogs, rabbits, etc. In a specific embodiment, an animal model such as described in Section 6, infra, is used to test the utility of an oncolytic virus described herein, such as an APMV described herein, or composition thereof to treat cancer.

### 5.8.6 Expression of Transgene

The expression of a protein in cells infected with a recombinant oncolytic virus, such as a recombinant APMV described herein, wherein the recombinant oncolytic virus comprises a packaged genome comprising a transgene encoding a heterologous protein, may be conducted using any assay known in the art, such as, e.g., western blot, immunofluorescence, flow cytometry, and ELISA, or any assay described herein (see, e.g., Section 6). In a specific embodiment, an assay described in Section 6, infra, is used to assess transgene expression.

In a specific aspect, an ELISA is utilized to detect expression of a heterologous protein encoded by a transgene in cells infected with a recombinant oncolytic virus, such as a recombinant APMV described herein, comprising a packaged genome comprising the transgene.

The expression of a transgene may also be measured at the RNA level by assays, such as Northern blots and quantitative RT-PCR, known to one of skill in the art.

In addition to expression of a transgene, the function of the protein encoded by the transgene may be assessed by techniques known to one of skill in the art. For example, one or more functions of a protein described herein or known to one of skill in the art may be assessed using techniques known to one of skill in the art.

### 5.9 Kits

In one aspect, provided herein is a pharmaceutical pack or kit comprising one or more containers filled with one or more of the ingredients of a composition (e.g., a pharmaceutical compositions) described herein. In a specific embodiment, provided herein is a pharmaceutical pack or kit comprising a container, wherein the container comprises an oncolytic virus described herein, such as an APMV (e.g., AMPV-1 or APMV-4), or a pharmaceutical composition comprising an oncolytic virus described herein, such as an APMV (e.g., AMPV-1 or APMV-4). In a particular embodiment, provided herein is a pharmaceutical pack or kit comprising a container, wherein the container comprises an APMV-1 described herein, or a pharmaceutical composition comprising an APMV-1 described herein. In another particular embodiment, provided herein is a pharmaceutical pack or kit comprising a container, wherein the container comprises an APMV-4 described herein, or a pharmaceutical composition comprising an APMV-4 described herein. In some embodiments, the pharmaceutical pack or kit comprises a second container, wherein the second container comprises a VEGFR-3-activating agent. In certain embodiments, the pharmaceutical pack or kit comprises a second container, wherein the second container comprises a VEGF-C agent. In some embodiments, the pharmaceutical pack or kit comprises a second container, wherein the second container comprises a VEGF-D agent. In certain embodiments, the pharmaceutical pack or kit comprises a second container, wherein the second container comprises a VEGF-C agent and a VEGF-D agent. In some embodiments, the pharmaceutical pack or kit comprises an additional container, wherein the second container comprises an additional prophylactic or therapeutic agent, such as, e.g., described in Section 5.7.2. Optionally associated with such container(s) can be a notice in the form prescribed by a governmental agency regulating the manufacture, use or sale of pharmaceuticals or biological products, which notice reflects approval by the agency of manufacture, use or sale for human administration. In a specific embodiment, the pharmaceutical pack or kit includes instructions for use of the oncolytic virus (e.g., APMV) or composition thereof and/or VEGFR-3-activating agent or composition thereof, VEGF-C agent or composition thereof or VEGF-D agent or composition thereof for the treatment of cancer. For example, the instructions may describe the methods for treating cancer described herein.

### 5.10 Sequences

TABLE 1
Primer Sequences.

| Primer | | SEQ |
|---|---|---|
| name | Sequence (5′-3′) | ID NO. |
| mVEGFC-fwd | ACCGAGTTCCCCCCCccgcgg TTAGAAAAAA T | 65 |
| | ACGGGTAGAA CCGCCACC ATG CAT CTG CTG TGT TTC | |
| | CTG TC | |
| mVEGFC-rev | TTGGACCTTGGGTCCgcggg CTA ATT CAG ATG AGG TCG | 66 |
| | CTT CCA GTA T | |
| APMV4- | CCAAGCTTGCATGCC ACG AAA AAG AAG AAT AAA AGG | 67 |
| PCR1-1-F | CA | |
| APMV4- | GGG CGC GCC ACT GAG TCT T | 68 |
| PCR1-1-R | | |
| APMV4- | CTC AGT GGC GCG CCC CA | 69 |
| PCR1-2-F | | |
| APMV4- | GAT GTC GAC GGA CGG TGT G | 70 |
| PCR1-2-R | | |
| APMV4- | CCG TCC GTC GAC ATC CCT | 71 |
| PCR1-3-F | | |
| APMV4- | CGG TAC CCG GGG ATC CAT CAC CTG CAG GAT TAC AT | 72 |
| PCR1-3-R | | |
| APMV4- | CCAAGCTTGCATGCC taatcctgcaggtgatgaatctg | 73 |
| PCR2-1-F | | |
| APMV4- | GTT CGA TCG TTT TTA ATT AAA AAG G | 74 |
| PCR2-1-R | | |
| APMV4- | TAA AAA CGA TCG AAC TGA GG | 75 |
| PCR2-2-F | | |
| APMV4- | CGG TAC CCG GGG ATC ATT TTA CGG CCG CTC AGG G | 76 |
| PCR2-2-R | | |
| APMV4- | CCAAGCTTGCATGCC GAG CGG CCG TAA AAT TAA CAC | 53 |
| PCR3-F | | |
| APMV4-125-R | GAT TAT CTA GAT TGT CAG AAC CCA TAA AGA ATT TGG | 54 |
| APMV4-125-F | ACA ATC TAG ATA ATC TTG ATA TCT ACC AGC AGC | 55 |
| APMV4- | CGG TAC CCG GGG ATC AAG AAA TAA AAG ACA TAT TTT | 56 |
| PCR3-R | TTA TTA AAT ATT AAT ACG | |
| InF-N- | CACGATAATACCATGGCTGGTGTCTTCTCCCAGTATG | 57 |
| APMV4-F | | |
| InF-N- | TTAGGCCTCTCGAGCCTGCAGCTACAGTTCAAAGTCGGGTT | 58 |
| APMV4-R | GATAGTC | |
| InF-P- | CACGATAATACCATGGATTTTACTGACATTGATGCTGTCAA | 59 |
| APMV4-F | CTC | |
| InF-P- | TTAGGCCTCTCGAGCCTGCAGCTAGAGCCCAAGGGCTTGT | 60 |
| APMV4-R | C | |
| InF-L- | CACGATAATACCATGTCCAATCAGGCAGCTGAGATTATAC | 61 |
| APMV4-F | | |
| InF-L- | TTAGGCCTCTCGAGCCTGCAGCTAAAGTGAGAGGTAGCCC | 62 |
| APMV4-R | CAACC | |
| APMV- | cacaccgtccgtcga C ATT TTT AAT TAA AAT AGG GTG GGG | 63 |
| mVEGFC-fwd | | |
| APMV- | GGC AAG GGA TGT CGA CCC GGT CAG TTC AG | 64 |
| mVEGFC-rev | | |

TABLE 2
APMV SEQUENCES

| | | SEQ |
|---|---|---|
| | | ID |
| Description | Sequence | NO. |
| Avian | GCGAAAAAGAAGAATAAAAGGCAGAAGCCTTTTAAAAGGAACCCTGGG | 77 |
| paramyxovirus | CTGTCGTAGGTGTGGGAAGGTTGTATTCCGAGTGCGCCTCCGAGGCATC | |
| 4 strain | TACTCTACACCTATCACAATGGCTGGTGTCTTCTCCCAGTATGAGAGGTT | |
| APMV4/mall | TGTGGACAATCAATCCCAAGTATCAAGGAAGGATCATCGGTCCCTGGCA | |
| ard/Belgium/ | GGGGGATGCCTTAAAGTCAACATCCCTATGCTTGTCACTGCATCTGAAG | |
| 15129/07 | ATCCCACCACTCGTTGGCAACTAGCATGTTTATCTCTAAGGCTCTTGATC | |
| complete | TCCAACTCATCAACCAGTGCTATCCGACAGGGGGCAATACTGACTCTCA | |
| genome | TGTCACTACCGTCACAAAATATGAGAGCAACGGCAGCTATTGCTGGTTC | |
| Genbank: | CACAAATGCAGCTGTTATCAACACTATGGAAGTCTTGAGTGTCAATGAC | |
| JN571485.1 | TGGACCCCATCCTTCGACCCTAGGAGCGGTCTCTCTGAAGAGGATGCTC | |
| | AGGTTTTCAGAGACATGGCAAGGGACCTGCCCCCTCAGTTCACCTCCGG | |
| | ATCACCCTTTACATCAGCATTGGCGGAGGGGTTTACCCCAGAAGACACC | |
| | CACGACCTAATGGAGGCCTTGACCAGTGTGCTGATACAGATCTGGATCC | |
| | TGGTGGCTAAGGCCATGACCAACATTGATGGCTCTGGGGAGGCCAATG | |
| | AGAGACGTCTTGCAAAGTACATCCAAAAGGGACAGCTTAATCGCCAGTT | |
| | TGCAATTGGTAATCCTGCTCGTCTGATAATCCAACAGACGATCAAAAGC | |
| | TCCTTAACTGTCCGCAGGTTCTTGGTCTCTGAGCTTCGTGCATCACGAGG | |
| | TGCAGTGAAAGAAGGATCCCCTTACTATGCAGCTGTTGGGGATATCCAC | |
| | GCTTACATCTTTAACGCAGGACTGACACCATTCTTGACTACCTTAAGAT | |
| | ATGGGATAGGCACCAAGTATGCTGCTGTTGCACTCAGTGTGTTCGCTGC | |
| | AGACATTGCAAAATTAAAGAGCCTACTTACCCTGTACCAAGACAAGGGT | |
| | GTGGAGGCCGGATACATGGCACTCCTTGAAGATCCAGATTCCATGCACT | |
| | TTGCACCCGGAAATTTCCCACACATGTACTCCTATGCGATGGGGGTGGC | |
| | TTCTTACCATGACCCCAGCATGCGCCAATACCAATATGCCAGGAGGTTC | |
| | CTCAGCCGTCCCTTCTACTTGCTAGGGAGGGACATGGCCGCCAAGAACA | |
| | CAGGCACGCTGGATGAGCAACTGGCAAAGGAACTGCAAGTGTCAGAAA | |
| | GAGACCGCGCCGCATTGTCCGCTGCGATTCAATCAGCAATGGAGGGGG | |
| | GAGAATCTGACGACTTCCCACTGTCGGGATCCATGCCGGCTCTCTCCGA | |
| | CACTGCGCAACCAGTTACCCCAAGAACCCAACAGTCCCAGCTTTCCCCT | |
| | CCACAATCATCAAGCATGTCTCAATCAGCGCCCAGGACCCCGGACTACC | |
| | AGCCTGATTTTGAACTGTAGGCTGCATCCACGCACCAACAACAGGCAAA | |
| | AGAAATCACCCTCCTCCCCACACATCCCACCCACTCACCCGCCGAGATC | |
| | CAATCCAACACCCCAGCATCCCCATCATTTAATTAAAAACTGACCAATA | |
| | GGGTGGGGAAGGAGAGTTATTGGCTGTTGCCAAGTTTGTGCAGCAATGG | |
| | ATTTCACCGACATTGATGCTGTCAACTCATTAATTGAATCATCATCAGCA | |
| | ATCATAGATTCCATACAGCATGGAGGGCTGCAACCATCGGGCACTGTCG | |
| | GCCTATCGCAAATCCCAAAGGGGATAACCAGCGCTTTAACTAAGGCCTG | |
| | GGAGGCTGAGGCAGCAACTGCTGGCAATGGGGACACCCAACACAAACC | |
| | TGACAGTCCGGAGGATCATCAGGCCAACGACACAGACTCCCCCGAAGA | |
| | CACAGGCACCAACCAGACCATCCAGGAAGCCAATATCGTTGAAACACC | |
| | CCACCCCGAAGTGCTATCGGCAGCCAAAGCCAGACTCAAGAGGCCCAA | |
| | GGCAGGGAGGGACACCCACGACAATCCCTCTGCGCAACCTGATCATTTT | |
| | TTAAAGGGGGGCCCCCTGAGCCCACAACCAGCGGCACCATGGGTGCAA | |
| | AGTCCACCCATTCATGGAGGTCCCGGCACCGTCGATCCCCGCCCATCAC | |
| | AAACTCAGGATCATTCCCTCACCGGAGAGAAATGGCAATCGTCACCGAC | |
| | AAAGCAACCGGAGACATTGAACTGGTGGAATGGTGCAACCCGGGGTGC | |
| | ACCGCAATCCGAACTGAACCAACCAGACTCGACTGTGTATGCGGACACT | |
| | GCCCCACCATCTGCAGCCTCTGCATGTATGACGACTGATCAGGTACAAC | |
| | TATTAATGAAGGAGGTTGCCGATATGAAATCACTCCTTCAGGCATTAGT | |
| | AAAGAACCTAGCTGTCCTGCCTCAACTAAGGAACGAGGTTGCAGCAATC | |
| | AGGACATCACAGGCCATGATAGAGGGGACACTCAATTCAATCAAGATT | |
| | CTCGATCCTGGGAATTATCAAGAATCATCACTAAACAGCTGGTTCAAAC | |
| | CACGCCAAGATCACGCGGTTGTTGTGTCCGGACCAGGGAATCCATTGAC | |
| | CATGCCAACCCCAATCCAAGACAACACCATATTCCTGGATGAACTGGCA | |
| | AGACCTCATCCTAGTTTGGTCAATCCGTCCCCGCCCACTACCAACACTA | |
| | ATGTTGATCTTGGCCCACAGAAGCAGGCTGCGATAGCTTATATCTCAGC | |
| | AAAATGCAAGGATCCAGGGAAACGAGATCAGCTCTCAAAGCTCATCGA | |
| | GCGAGCAACCACCTTGAGCGAGATCAACAAAGTCAAAAGACAGGCCCT | |
| | CGGCCTCTAGATCACTCGACCACCCCCAGTAATGAATACAACAATAATC | |
| | AGAACCCCCCTAAAACACATGGTCAACCCAACACACCACCCGCACCAC | |
| | CCGCTACTATCCTTTGCCAGAAACTCCGCCGCAGCCGATTTATTCAAAA | |
| | GAAGCCATTTGATATGACTTAGCAACCGCAAGATAGGGTGGGGAAGGT | |
| | GCTTTGCCTGCAAGAGGGCTCCCTCATCTTCAGACACGTACCCGCCAAC | |
| | CCACCAGTGACGCAATGGCAGACATGGACACCGTATATATCAATCTGAT | |
| | GGCAGATGATCCAACCCACCAAAAAGAACTGCTGTCCTTTCCCCTCGTT | |
| | CCCGTGACTGGTCCTGACGGGAAAAAGGAACTCCAACACCAGGTCCGG | |
| | ACTCAATCCTTGCTCGCCTCAGACAAGCAAACTGAGAGGTTCATCTTCC | |
| | TCAACACTTACGGGTTTATCTATGACACTACACCGGACAAGACAACTTT | |
| | TTCCACCCCAGAGCACATCAATCAGCCCAAGAGAACGATGGTGAGTGCT | |
| | GCGATGATGACCATTGGCCTGGTCCCCGCCAATATACCCTTGAACGAAT | |
| | TAACAGCTACTGTGTTCGGCCTGAAAGTAAGAGTGAGGAAGAGTGCGA | |
| | GATATCGAGAGGTGGTCTGGTATCAGTGCAATCCTGTACCAGCCCTGCT | |
| | TGCAGCCACCAGGTTCGGTCGCCAAGGAGGTCTCGAATCAAGCACTGG | |
| | AGTCAGCGTAAAGGCCCCCGAGAAGATAGATTGCGAGAAGGATTATAC | |
| | TTACTACCCTTATTTCCTATCTGTGTGCTACATCGCCACTTCCAACCTGTT | |
| | CAAGGTACCAAAAATGGTTGCTAATGCGACCAACAGTCAATTATACCAC | |
| | CTGACTATGCAGGTCACATTTGCCTTTCCAAAAAACATCCCCCCAGCTA | |
| | ACCAGAAACTTCTGACACAAGTGGATGAAGGATTCGAGGGCACTGTGG | |
| | ACTGCCATTTTGGGAACATGCTGAAAAAGGATCGGAAAGGGAATATGA | |
| | GGACATTGTCGCAGGCGGCAGACAAGGTCAGACGGATGAATATCCTTG | |
| | TTGGTATCTTTGACTTGCATGGGCCGACACTCTTCCTGGAGTATACTGGG | |
| | AAACTAACAAAAGCTCTGTTAGGGTTCATGTCTACTAGCCGAACAGCAA | |
| | TCATCCCCATATCTCAGCTCAATCCTATGCTGGGTCAACTTATGTGGAGC | |
| | AGTGATGCCCAGATAGTAAAATTAAGAGTGGTCATAACTACATCCAAAC | |
| | GCGGCCCATGCGGGGGTGAGCAGGAGTATGTGCTGGATCCCAAATTCA | |
| | CAGTTAAAAAAGAGAAAGCCCGACTCAACCCTTTCAAGAAGGCAGCCC | |
| | AATGATCAAATCTGCAGGATCTCAAGAATCAGACCACTCTATACTATTC | |
| | ACCGATCAATAGACATGTAACTATACAGTTGATGGACCTATGAAGAATC | |
| | AATTAGCAAACCGAATCCTTACTAGGGTGGGGAAGGAGTTGATTGGGT | |
| | GTCTAAACAAAAGCATTCCTTTACACCTCCTCGCTACGAAACAACCATA | |
| | ATGAGGTTATCACGCACAATCCTGACTTTGATTCTCAGCACACTTACCG | |
| | GCTATTTAATGAATGCCCACTCCACCAATGTGAATGAGAAACCAAAGTC | |
| | TGAGGGGATTAGGGGGGATCTTATACCAGGCGCAGGTATTTTTGTAACT | |
| | CAAGTCCGACAACTACAGATCTACCAACAGTCTGGGTATCATGACCTTG | |
| | TCATCAGGTTATTACCTCTTCTACCGGCAGAACTTAATGATTGTCAAAG | |
| | GGAAGTTGTCACAGAGTACAACAACACGGTATCACAGCTGTTGCAGCCT | |
| | ATCAAAACCAACCTGGATACCTTATTGGCTGATGGTAGCACAAGGGATG | |
| | CCGATATACAGCCACGGTTCATTGGGGCAATAATAGCCACAGGTGCCCT | |
| | GGCGGTGGCTACGGTAGCTGAGGTGACTGCAGCCCAAGCACTATCTCAG | |
| | TCGAAAACAAACGCTCAAAATATTCTCAAGTTGAGAGATAGTATTCAGG | |
| | CTACCAACCAAGCAGTTTTCGAAATTTCACAAGGACTCGAGGCAACTGC | |
| | AACTGTGCTATCAAAACTGCAAACTGAGCTCAATGAGAACATTATCCCA | |
| | AGCCTGAACAACTTGTCCTGTGCTGCCATGGGGAATCGCCTTGGTGTAT | |
| | CACTATCACTCTACTTGACCTTAATGACCACTCTATTTGGGGACCAGATC | |
| | ACAAACCCAGTGCTGACACCAATCTCCTATAGCACTCTATCGGCAATGG | |
| | CAGGCGGTCACATTGGCCCGGTGATGAGTAAAATATTAGCTGGATCTGT | |
| | CACAAGTCAGTTGGGGGCAGAACAGTTGATTGCTAGCGGCTTAATACAG | |
| | TCACAGGTAGTAGGTTATGATTCCCAATATCAATTATTGGTTATCAGGG | |
| | TCAACCTTGTACGGATTCAAGAGGTCCAGAATACGAGGGTCGTATCACT | |
| | AAGAACACTAGCGGTCAATAGGGATGGTGGACTTTATAGAGCCCAGGT | |
| | GCCTCCCGAGGTAGTTGAACGGTCTGGCATTGCAGAGCGATTTTATGCA | |
| | GATGATTGTGTTCTTACTACAACTGATTACATTTGCTCATCGATCCGATC | |
| | TTCTCGGCTTAATCCAGAGTTAGTCAAGTGTCTCAGTGGTGCACTTGATT | |
| | CATGCACATTTGAGAGGGAAAGTGCATTATTGTCGACCCCTTTCTTTGTA | |
| | TACAACAAGGCAGTCGTCGCAAATTGTAAAGCAGCAACATGTAGATGT | |
| | AATAAACCGCCATCTATTATTGCCCAATACTCTGCATCAGCTCTAGTCAC | |
| | CATCACCACCGACACCTGTGCCGACCTTGAAATTGAGGGTTATCGCTTC | |
| | AACATACAGACTGAATCCAACTCATGGGTTGCACCAAACTTCACGGTCT | |
| | CGACTTCACAGATTGTATCAGTTGATCCAATAGACATCTCCTCTGACATT | |
| | GCCAAAATCAACAGTTCCATCGAGGCTGCGAGAGAGCAGCTGGAACTG | |
| | AGCAACCAGATCCTTTCCCGGATCAACCCACGAATTGTGAATGATGAAT | |
| | CACTGATAGCTATTATCGTGACAATTGTTGTGCTTAGTCTCCTTGTAATC | |
| | GGTCTGATTGTTGTTCTCGGTGTGATGTATAAGAATCTTAAGAAAGTCC | |
| | AACGAGCTCAAGCTGCCATGATGATGCAGCAAATGAGCTCATCACAGC | |
| | CTGTGACCACTAAATTAGGGACGCCTTTCTAGGAGAATAATCATATCAC | |
| | TCTACTCAATGATGAGCAAAACGTACCAATCGTCAATGATTGTGTCACG | |
| | AGGCCGGTTGGGAATGCATCGAATCTCTCCCCTTTCTTTTTAATTAAAAA | |
| | CATTTGAAGTGAGGGTGAGAGGGGGGGAGTGTATGGTAGGGTGGGGAA | |
| | GGTAGCCAATTCCTGCCTATTGGGCCGACCGTATCAAAAGAACTCAACA | |
| | GAAGTCTAGATACAGGGTGACATGGAGGGCAGCCGTGATAATCTTACA | |
| | GTGGATGATGAATTAAAGACAACATGGAGGTTAGCTTATAGAGTTGTGT | |
| | CCCTTCTATTGATGGTGAGCGCTTTGATAATCTCTATAGTAATCCTGACA | |
| | AGAGATAACAGCCAAAGCATAATCACAGCGATCAACCAGTCATCCGAC | |
| | GCAGACTCAAAGTGGCAAACGGGAATAGAAGGGAAAATCACCTCCATT | |
| | ATGACTGATACGCTCGATACCAGGAATGCAGCCCTTCTCCACATTCCAC | |
| | TCCAGCTCAACACGCTTGAGGCGAACCTTTTGTCCGCCCTTGGGGGCAA | |
| | CACAGGAATTGGTCCCGGGGATCTAGATCACTGCCGTTACCCTGTTCAT | |
| | GACTCCGCTTACCTGCATGGAGTTAATCGATTACTCATCAACCAGACAG | |
| | CTGATTACACAGCAGAAGGCCCCCTAGATCATGTGAACTTTATTCCAGC | |
| | CCCGGTTACGACCACTGGATGCACAAGGATACCATCCTTTTCCGTGTCA | |
| | TCGTCCATTTGGTGCTATACACACAACGTGATCGAAACCGGTTGCAATG | |
| | ACCACTCAGGTAGTAACCAATATATCAGCATGGGAGTCATTAAGAGAG | |
| | CGGGCAACGGCCTACCTTACTTCTCGACAGTTGTAAGTAAATATCTGAC | |
| | TGATGGGTTGAATAGGAAAAGCTGTTCTGTAGCCGCCGGATCCGGGCAT | |
| | TGCTACCTCCTTTGCAGCTTAGTGTCGGAACCCGAACCTGATGACTATGT | |
| | GTCACCTGATCCCACACCGATGAGGTTAGGGGTGCTAACGTGGGATGGG | |
| | TCTTACACTGAACAGGTGGTACCCGAAAGAATATTCAAGAACATATGGA | |
| | GTGCAAACTACCCAGGAGTAGGGTCAGGTGCTATAGTAGGGAATAAGG | |
| | TGTTATTCCCATTTTACGGCGGAGTGAGAAATGGATCGACCCCGGAGGT | |
| | GATGAATAGGGGAAGATACTACTACATCCAGGATCCAAATGACTATTGT | |
| | CCTGACCCGCTACAAGATCAGATCTTAAGGGCGGAACAATCGTATTACC | |
| | CAACTCGATTTGGTAGGAGGATGGTAATGCAAGGGGTCCTAGCATGTCC | |
| | AGTATCCAACAATTCAACAATAGCAAGCCAATGTCAATCTTACTATTTT | |
| | AATAACTCATTAGGATTCATTGGGGCAGAATCTAGAATCTATTACCTCA | |
| | ATGGTAACATTTACCTTTATCAGAGAAGCTCGAGCTGGTGGCCTCATCC | |
| | CCAGATTTACCTGCTTGATTCCAGGATTGCAAGTCCGGGTACTCAGAAC | |
| | ATTGACTCAGGTGTTAATCTCAAGATGTTAAATGTTACTGTGATTACAC | |
| | GACCATCATCTGGTTTTTGTAATAGTCAGTCACGATGCCCTAATGACTGC | |
| | TTATTCGGGGTCTACTCGGATATCTGGCCTCTTAGCCTTACCTCAGATAG | |
| | CATATTCGCGTTCACAATGTATTTACAGGGGAAGACAACACGTATTGAC | |
| | CCGGCTTGGGCACTATTCTCCAATCATGCGATTGGGCATGAGGCTCGTC | |
| | TGTTCAATAAGRAGGTTAGTGCTGCTTATTCTACCACCACTTGTTTTTCG | |
| | GACACTATCCAAAATCAGGTGTATTGCCTGAGTATACTTGAGGTCAGGA | |
| | GTGAGCTCTTGGGAGCATTCAAAATAGTACCATTCCTCTATCGCGTCTTG | |
| | TAGGCATCCATTCAGCCAAAAAACTTGAGTGACCATGAGGTTAACACCT | |
| | GATCCCCTTCAAAAACATCTATCTTAATTACCGTTCTAGATCCATGATTA | |
| | GGTACCTTTCCAATCAATCATTTGGTTTTTAATTAAAAACGAAAGAATG | |
| | GGCCTAGTTCCAAGAAAGGGCTGGAACCCATTAGGGTGGGGAAGGATT | |
| | GCTTTGCTCCTTGACTCACACCTGCGTACACTCGATCTCACTTCTATAAA | |
| | GAAGGAATCCTTCTCAAATTCGCCCCACAATGTCCAATCAGGCAGCTGA | |
| | GATTATACTACCCACCTTCCATCTAGAATCACCCTTAATCGAGAATAAG | |
| | TGCTTCTATTATATGCAATTACTTGGTCTCGTGTTGCCACATGATCACTG | |
| | GAGATGGAGGGCATTCGTTAACTTTACAGTGGATCAGGTGCACCTTAAA | |
| | AATCGTAATCCCCGCTTAATGGCCCACATCGACCACACTAAAGATAGAT | |
| | TAAGGACTCATGGTGTCTTAGGTTTCCACCAGACTCAGACAAGTATGAG | |
| | CCGTTACCGTGTTTTGCTTCATCCTGAAACCTTACCTTGGCTATCAGCCA | |
| | TGGGAGGATGCATCAATCAGGTTCCTAAAGCATGGCGGAACACTCTGA | |
| | AATCGATCGAGCACAGTGTAAAGCAGGAGGCACCTCAACTAAAGTTAC | |
| | TCATGGAGAGAACCTCATTAAAATTAACTGGAGTACCTTACTTGTTCTCT | |
| | AATTGCAATCCCGGGAAAACCACAGCAGGAACTATGCCTGTCCTAAGTG | |
| | AGATGGCATCGGAACTCTTATCAAATCCTATCTCCCAATTCCAATCAAC | |
| | ATGGGGGTGTGCTGCTTCGGGGTGGCACCATGTAGTCAGTATCATGAGG | |
| | CTCCAACAATATCAAAGAAGGACAGGTAAGGAAGAGAAAGCAATCACT | |
| | GAAGTTCAGTATGGCACGGACACCTGTCTCATTAACGCAGACTACACCG | |
| | TTGTTTTTTCCACACAGAACCGTGTTATAACGGTCTTGCCTTTCGATGTT | |
| | GTCCTCATGATGCAAGACCTGCTAGAATCCCGACGGAATGTCCTGTTCT | |
| | GTGCCCGCTTTATGTATCCCAGAAGCCAACTTCATGAGAGGATAAGTAC | |
| | AATATTAGCCCTTGGAGACCAACTGGGGAGAAAAGCACCCCAAGTCCT | |
| | GTATGATTTTGTAGCAACCCTTGAGTCATTTGCATACGCAGCTGTTCAAC | |
| | TTCATGACAACAATCCTACCTACGGTGGGGCCTTCTTTGAATTCAATATC | |
| | CAAGAGTTAGAATCTATTCTGTCCCCTGCACTTAGTAAGGATCAGGTCA | |
| | ACTTCTACATAGGTCAAGTTTGCTCAGCGTACAGTAACCTTCCTCCATCT | |
| | GAATCGGCAGAATTGCTGTGCCTGCTACGCCTGTGGGGTCATCCCTTGC | |
| | TAAACAGCCTTGATGCAGCAAAGAAAGTCAGGGAATCTATGTGTGCCG | |
| | GGAAGGTTCTCGATTACAACGCCATTCGACTCGTCTTGTCTTTTTATCAT | |
| | ACGTTACTAATCAATGGGTATCGGAAGAAGCACAAGGGTCGCTGGCCA | |
| | AATGTGAATCAACATTCACTCCTCAACCCGATAGTGAGGCAGCTTTATT | |
| | TTGATCAGGAGGAGATCCCACACTCTGTTGCCCTTGAGCACTATTTGGA | |
| | TGTCTCAATGATAGAATTTGAGAAAACTTTTGAAGTGGAACTATCTGAC | |
| | AGCCTAAGCATCTTCCTGAAGGATAAGTCGATAGCTTTGGACAAGCAAG | |
| | AATGGTACAGTGGTTTTGTCTCAGAAGTGACTCCGAAGCACCTGCGAAT | |
| | GTCCCGTCATGATCGCAAGTCTACCAATAGGCTCCTGTTAGCCTTCATTA | |
| | ACTCCCCTGAATTCGATGTTAAGGAAGAGCTTAAATACTTGACTACGGG | |
| | TGAGTACGCTACTGACCCAAATTTCAATGTCTCTTACTCACTCAAAGAG | |
| | AAGGAAGTAAAGAAAGAAGGGCGCATTTTCGCAAAAATGTCACAAAAG | |
| | ATGAGAGCATGCCAGGTTATTTGTGAAGAATTGCTAGCACATCATGTGG | |
| | CTCCTTTGTTTAAAGAGAATGGTGTTACTCAATCGGAGCTATCCCTGAC | |
| | AAAAAATTTGTTGGCTATTAGCCAACTGAGTTACAACTCGATGGCCGCT | |
| | AAGGTGCGATTGCTGAGGCCAGGGGACAAGTTCACTGCTGCACACTATA | |
| | TGACCACAGACCTAAAGAAGTACTGTCTCAATTGGCGGCACCAGTCAGT | |
| | CAAACTGTTCGCCAGAAGCCTGGATCGACTGTTTGGGCTAGACCATGCT | |
| | TTTTCTTGGATACATGTCCGTCTCACCAACAGCACTATGTACGTTGCTGA | |
| | CCCCTTCAATCCACCAGACTCAGATGCATGCACAAACTTAGACGACAAT | |
| | AAGAACACCGGGATTTTTATTATAAGTGCACGAGGTGGTATAGAAGGCC | |
| | TCCAACAAAAACTATGGACTGGCATATCAATCGCAATTGCCCAAGCAGC | |
| | AGCAGCCCTCGAAGGCTTACGAATTGCTGCTACTCTGCAGGGGGATAAC | |
| | CAAGTTTTGGCGATTACAAAGGAGTTCATGACCCCAGTCCCGGAGGATG | |
| | TAATCCATGAGCAGCTATCTGAGGCGATGTCCCGATACAAAAGGACTTT | |
| | CACATACCTCAATTATTTAATGGGGCATCAGTTGAAGGATAAGGAAACC | |
| | ATCCAATCCAGTGATTTCTTTGTGTACTCCAAAAGAATCTTCTTCAATGG | |
| | ATCAATCTTAAGTCAATGCCTCAAGAACTTCAGTAAACTCACTACTAAT | |
| | GCCACTACCCTTGCTGAGAACACTGTGGCCGGCTGCAGTGACATCTCTT | |
| | CATGCATTGCCCGTTGTGTGGAAAACGGGTTGCCTAAGGATGCCGCATA | |
| | TATTCAGAATATAATCATGACTCGGCTTCAACTATTGCTAGATCATTACT | |
| | ATTCAATGCATGGCGGCATAAACTCAGAATTAGAGCAGCCAACTTTAAG | |
| | TATCTCTGTTCGAAACGCGACCTACTTACCATCTCAACTAGGCGGTTAC | |
| | AATCATTTGAATATGACCCGACTATTCTGCCGCAATATCGGCGACCCGC | |
| | TTACCAGTTCTTGGGCGGAGTCAAAAAGACTAATGGATGTTGGCCTTCT | |
| | CAGTCGTAAGTTCTTAGAGGGGATATTATGGAGACCCCCGGGAAGTGG | |
| | GACATTTTCAACACTCATGCTTGATCCGTTCGCACTTAACATTGATTACC | |
| | TGAGGCCGCCAGAGACAATTATCCGAAAACACACCCAAAAAGTCTTGTT | |
| | GCAAGATTGCCCAAATCCCCTATTAGCAGGTGTCGTTGACCCGAACTAC | |
| | AACCAAGAATTAGAGCTATTAGCTCAGTTCTTGCTTGATCGGGAAACCG | |
| | TTATCCCCAGGGCTGCCCATGCCATCTTTGAATTGTCTGTCTTGGGAAGG | |
| | AAAAAACATATACAAGGATTGGTAGATACTACAAAAACAATTATTCAG | |
| | TGCTCATTGGAAAGACAGCCATTGTCCTGGAGGAAAGTTGAGAACATTG | |
| | TTACCTACAACGCGCAGTATTTCCTCGGGGCCACCCAACAGGCTGATAC | |
| | TAATGTCTCAGAAGGGCAGTGGGTGATGCCAGGTAACTTCAAGAAGCTT | |
| | GTGTCCCTTGACGATTGCTCAGTCACGTTGTCCACTGTATCGCGGCGCAT | |
| | ATCGTGGGCCAATCTACTGAACTGGAGAGCTATAGATGGTTTAGAAACC | |
| | CCGGATGTGATAGAGAGTATTGATGGCCGCCTTGTACAATCATCCAATC | |
| | AATGTGGCCTATGTAATCAAGGGTTGGGATCCTACTCCTGGTTCTTCTTG | |
| | CCCTCTGGGTGTGTGTTCGACCGTCCACAAGATTCTCGGGTAGTTCCAA | |
| | AGATGCCATACGTGGGGTCCAAAACAGATGAGAGACAGACTGCATCAG | |
| | TGCAAGCTATACAGGGATCCACTTGTCACCTCAGAGCAGCATTGAGGCT | |
| | TGTATCACTCTATCTATGGGCCTATGGAGATTCTGACATATCATGGCTAG | |
| | AAGCTGCGACACTGGCTCAAACACGGTGCAATGTTTCTCTTGATGACTT | |
| | GCGAATCTTGAGCCCTCTCCCTTCTTCGGCGAATTTACACCACAGATTAA | |
| | ATGACGGGGTAACACAGGTTAAATTCATGCCCGCCACATCGAGCCGAGT | |
| | GTCAAAGTTCGTCCAAATTTGCAATGACAACCAGAATCTTATCCGTGAT | |
| | GATGGGAGTGTTGATTCCAATATGATTTATCAACAAGTTATGATATTGG | |
| | GGCTTGGAGAGATTGAATGCTTGCTAGCTGACCCAATCGATACAAACCC | |
| | AGAACAATTGATTCTTCATCTACACTCTGATAATTCTTGCTGTCTCCGGG | |
| | AGATGCCAACGACCGGCTTTGTACCTGCTCTAGGACTAACCCCATGTTT | |
| | AACTGTCCCAAAGCACAATCCTTACATTTATGATGATAGCCCAATACCC | |
| | GGTGATTTGGACCAGAGGCTCATCCAGACCAAATTTTTCATGGGTTCTG | |
| | ACAATTTGGATAATCTTGATATCTACCAACAGCGGGCTTTATTGAGTAG | |
| | GTGTGTGGCTTATGATGTTATCCAATCGATATTTGCTTGTGATGCACCAG | |
| | TCTCTCAGAAGAATGACGCAATCCTTCACACTGACTATCATGAGAATTG | |
| | GATCTCAGAGTTCCGATGGGGTGACCCTCGTATTATCCAAGTAACGGCA | |
| | GGCTACGAGTTAATTCTGTTCCTTGCATACCAGCTTTATTATCTCAGAGT | |
| | GAGGGGTGACCGTGCAATCCTATGTTATATTGACAGGATACTCAACAGG | |
| | ATGGTATCTTCCAATCTAGGCAGTCTCATCCAGACACTCTCTCATCCAGA | |
| | GATTAGGAGGAGATTCTCATTGAGTGATCAAGGGTTCCTTGTTGAAAGG | |
| | GAGCTAGAGCCAGGTAAGCCCTTGGTTAAACAAGCGGTTATGTTCTTGA | |
| | GGGACTCGGTCCGCTGCGCTTTAGCAACTATCAAGGCAGGAATTGAGCC | |
| | TGAGATCTCCCGAGGTGGCTGTACTCAGGATGAGCTGAGCTTTACTCTT | |
| | AAGCACTTACTGTGTCGGCGTCTCTGTGTAATCGCTCTCATGCATTCAGA | |
| | AGCAAAGAACTTGGTTAAAGTTAGAAACCTTCCTGTAGAAGAGAAAAC | |
| | CGCCTTACTGTACCAGATGTTGGTCACTGAGGCCAATGCTAGGAAATCA | |
| | GGATCTGCTAGCATCATCATAAATCTAGTCTCGGCACCCCAGTGGGACA | |
| | TTCATACACCAGCATTGTATTTTGTATCAAAGAAAATGCTAGGGATGCT | |
| | TAAAAGGTCAACCACACCCTTGGATATAAGTGACCTCTCCGAGAGCCAG | |
| | AATCCCGCACTTGCAGAGCTGAATGATGTTCCCGGTCACATGGCAGAAG | |
| | AATTTCCCTGTTTGTTTAGTAGTTATAACGCCACATATGAAGACACAATT | |
| | ACTTACAATCCAATGACTGAAAAACTCGCCTTACACTTGGACAACAGTT | |
| | CCACCCCATCCAGAGCACTTGGTCGTCACTACATCCTGCGGCCTCTTGG | |
| | GCTCTACTCATCCGCATGGTACCGGTCTGCAGCACTACTAGCGTCAGGG | |
| | GCCCTAAATGGGTTGCCTGAGGGGTCGAGCCTGTACCTAGGAGAAGGG | |
| | TACGGGACCACCATGACTCTGCTTGAGCCCGTTGTCAAGTCTTCAACTG | |
| | TTTACTACCATACATTGTTTGACCCAACCCGGAATCCTTCACAGCGGAA | |
| | CTATAAACCAGAACCACGGGTATTCACGGATTCTATTTGGTACAAGGAT | |
| | GATTTCACACGGCCACCTGGTGGTATTATCAATCTGTGGGGTGAAGATA | |
| | TACGTCAGAGTGATATCACACAGAAAGACACGGTCAACTTCATACTATC | |
| | TCAGATCCCGCCAAAATCACTTAAGTTGATACACGTTGATATTGAGTTC | |
| | TCACCAGACTCCGATGTACGGACACTACTATCTGGCTATTCTCATTGTGC | |
| | ACTATTGGCCTACTGGCTATTGCAACCTGGAGGGCGATTTGCAGTTAGA | |
| | GTTTTCTTAAGTGACCATATCATAGTAAACTTGGTCACTGCAATCCTGTC | |
| | TGCTTTTGACTCTAATCTGGTGTGCATTGCATCAGGATTGACACACAAG | |
| | GATGATGGGGCAGGTTATATTTGCGCAAAAAAGCTTGCAAATGTTGAGG | |
| | CTTCAAGGATCGAGTACTACTTGAGGATGGTCCATGGTTGTGTTGACTC | |
| | ATTAAAGATCCCTCATCAATTAGGAATCATTAAATGGGCCGAGGGCGAG | |
| | GTGTCCCAACTTACCAGAAAGGCGGATGATGAAATAAATTGGCGGTTA | |
| | GGTGATCCAGTTACCAGATCATTTGATCCAGTTTCTGAGCTAATAATTGC | |
| | ACGAACAGGGGGGTCTGTATTAATGGAATACGGGGCTTTTACTAACCTC | |
| | AGGTGTGCGAACTTGGCAGATACATACAAACTTCTGGCTTCAATTGTAG | |
| | AGACCACCCTAATGGAAATAAGGGTTGAGCAAGATCAATTAGAAGATA | |
| | ATTCGAGGAGACAAATCCAAGTAGTTCCCGCTTTCAACACTAGATCTGG | |
| | GGGAAGGATCCGTACGCTGATTGAGTGTGCTCAGCTGCAGATTATAGAT | |
| | GTTATTTGTGTAAACATAGATCACCTCTTTCCTAAACACCGACATGTTCT | |
| | TGTCACACAACTTACCTACCAGTCAGTGTGCCTTGGGGACTTGATTGAA | |
| | GGCCCCCAAATTAAGACGTATCTAAGGGCCAGGAAGTGGATCCAACGT | |
| | CAGGGACTCAATGAGACAGTTAACCATATCATCACTGGACAAGTGTCGC | |
| | GGAATAAAGCAAGGGATTTTTTCAAGAGGCGTCTGAAGTTGGTTGGCTT | |
| | TTCACTCTGCGGTGGTTGGAGCTACCTCTCACTTTAGCTGTTCAGGTTGT | |
| | TGATTATTATGAATAATCGGAGTCGGAATCGTAAATAGGAAGTCACAAA | |
| | GTTGTGAATAAACAATGATTGCATTAGTATTTAATAAAAAATATGTCTT | |
| | TTATTTCGT | |
| Avian | ACGAAAAAGAAGAATAAAAGGCAGAAGCCTTTTAAAAGGAACCCTGGG | 78 |
| paramyxovirus | CTGTCGTAGGTGTGGGAAGGTTGTATTCCGAGTGCGCCTCCGAGGCATC | |
| 4 APMV- | TACTCTACACCTATCACAATGGCTGGTGTCTTCTCCCAGTATGAGAGGTT | |
| 4/duck/Hong | TGTGGACAATCAATCCCAAGTGTCAAGGAAGGATCATCGGTCCTTAGCA | |
| kong/D3/75, | GGAGGATGCCTTAAAGTTAACATCCCTATGCTTGTCACTGCATCTGAAG | |
| complete | ACCCCACCACTCGTTGGCAACTAGCATGCTTATCTCTAAGGCTCCTGATC | |
| genome | TCCAACTCATCAACCAGTGCTATCCGTCAGGGGGCAATACTGACTCTCA | |
| Genbank: | TGTCATTACCATCACAAAACATGAGAGCAACAGCAGCTATTGCTGGTTC | |
| FJ177514.1 | CACAAATGCAGCTGTTATCAACACCATGGAAGTCTTAAGTGTCAACGAC | |
| | TGGACCCCATCCTTCGACCCTAGGAGCGGTCTTTCTGAGGAAGATGCTC | |
| | AAGTTTTCAGAGACATGGCAAGAGATCTGCCCCCTCAGTTCACCTCTGG | |
| | ATCACCCTTCACATCAGCATTGGCGGAGGGGTTCACTCCTGAAGATACT | |
| | CATGACCTGATGGAGGCCTTGACCAGTGTGCTGATACAGATCTGGATCC | |
| | TGGTGGCTAAGGCCATGACCAACATTGACGGCTCTGGGGAGGCCAATG | |
| | AAAGACGTCTTGCAAAGTACATCCAAAAAGGACAGCTTAATCGTCAGTT | |
| | TGCAATTGGTAATCCTGCCCGTCTGATAATCCAACAGACAATCAAAAGC | |
| | TCCTTAACTGTCCGTAGGTTCTTGGTCTCTGAGCTTCGTGCGTCACGAGG | |
| | TGCAGTAAAAGAAGGATCCCCTTACTATGCAGCTGTTGGGGATATCCAC | |
| | GCTTACATCTTTAATGCGGGATTGACACCATTCTTGACCACCTTAAGATA | |
| | CGGGATAGGCACCAAGTACGCCGCTGTTGCACTCAGTGTGTTCGCTGCA | |
| | GATATTGCAAAGTTGAAGAGCCTACTTACCCTGTACCAGGACAAGGGTG | |
| | TAGAAGCTGGATACATGGCACTCCTTGAGGATCCAGACTCCATGCACTT | |
| | TGCACCTGGAAACTTCCCACACATGTACTCCTATGCAATGGGGGTAGCT | |
| | TCTTACCATGATCCTAGCATGCGCCAATACCAATACGCCAGGAGGTTCC | |
| | TCAGCCGTCCTTTCTACTTACTAGGAAGGGACATGGCCGCCAAGAACAC | |
| | AGGCACGCTGGATGAGCAACTGGCGAAGGAACTGCAAGTATCAGAGAG | |
| | AGATCGCGCCGCATTATCCGCTGCGATTCAATCAGCGATGGAGGGGGG | |
| | AGAGTCCGACGACTTCCCACTGTCGGGATCCATGCCGGCTCTCTCTGAG | |
| | AATGCGCAACCAGTTACCCCCAGACCTCAACAGTCCCAGCTCTCTCCCC | |
| | CCCAATCATCAAACATGCCCCAATCAGCACCCAGGACCCCAGACTATCA | |
| | ACCCGACTTTGAACTGTAGGCTTCATCACCGCACCAACAACAGCCCAAG | |
| | AAGACCACCCCTCCCCCCACACATCTCACCCAGCCACCCATAAAGACTC | |
| | AGTCCCACGCCCCAGCATCTCCTTCATTTAATTAAAAACCGACCAACAG | |
| | GGTGGGGAAGGAGAGTCATTGGCTACTGCCAATTGTGTGCAGCAATGG | |
| | ATTTTACTGACATTGATGCTGTCAACTCATTGATCGAATCATCATCGGCA | |
| | ATCATAGACTCCATACAGCATGGAGGGCTGCAACCAGCGGGCACCGTC | |
| | GGCCTATCGCAGATCCCAAAAGGGATAACCAGCGCATTAACCAAGGCC | |
| | TGGGAGGCTGAGGCGGCAACTGCCGGTAATGGGGACACCCAACACAAA | |
| | TCTGACAGTCCGGAGGATCATCAGGCCAACGACACAGATTCCCCTGAAG | |
| | ACACAGGTACTGACCAGACCACCCAGGAGGCCAACATCGTTGAGACAC | |
| | CCCACCCCGAGGTGCTGTCAGCAGCCAAAGCCAGACTCAAGAGGCCCA | |
| | AAGCAGGGAGGGACACCCGCGACAACTCCCCTGCGCAACCCGATCATC | |
| | TTTTAAAGGGGGGCCTCCTGAGCCCACAACCAGCAGCATCATGGGTGCA | |
| | AAATCCACCCAGTCATGGAGGTCCCGGCACCGCCGATCCCCGCCCATCA | |
| | CAAACTCAGGATCATTCCCCCACCGGAGAGAAATGGCGATTGTCACCGA | |
| | CAAAGCAACCGGAGACATTGAACTGGTGGAGTGGTGCAACCCGGGGTG | |
| | CACAGCAGTCCGAATTGAACCCACCAGACTCGACTGTGTATGCGGACAC | |
| | TGCCCCACCATCTGTAGCCTCTGCATGTATGACGACTGATCAGGTACAA | |
| | CTACTAATGAAGGAGGTTGCTGACATAAAATCACTCCTTCAGGCGTTAG | |
| | TGAGGAACCTCGCTGTCTTGCCCCAATTGAGGAATGAGGTTGCAGCAAT | |
| | CAGAACATCACAGGCCATGATAGAGGGGACACTCAATTCGATCAAGAT | |
| | TCTTGACCCTGGGAATTATCAGGAATCATCACTAAACAGTTGGTTCAAA | |
| | CCTCGCCAAGATCACACTGTTGTTGTGTCTGGACCAGGGAATCCATTGG | |
| | CCATGCCAACCCCAGTCCAAGACAACACCATATTCCTGGACGAGCTAGC | |
| | CAGACCTCATCCTAGTGTGGTCAATCCTTCCCCACCCATCACCAACACC | |
| | AATGTTGACCTTGGCCCACAGAAGCAGGCTGCAATAGCCTATATCTCCG | |
| | CTAAATGCAAGGATCCGGGGAAACGAGATCAGCTATCAAGGCTCATTG | |
| | AGCGAGCAACCACCCCAAGTGAGATCAACAAAGTTAAAAGACAAGCCC | |
| | TTGGGCTCTAGATCACTCGATCACCCCTCATGGTGATCACAACAATAAT | |
| | CAGAACCCTTCCGAACCACATGACCAACCCAGCCCACCGCCCACACCGT | |
| | CCATCGACATCCCTTGCCAAACATCCTGCCGTAGCTGATTTATTCAAAA | |
| | GAGCTCATTTGATATGACCTGGTAATCATAAAATAGGGTGGGGAAGGTG | |
| | CTTTGCCTGTAAGGGGGCTCCCTCATCTTCAGACACGTGCCCGCCATCTC | |
| | ACCAACAGTGCAATGGCAGACATGGACACGGTGTATATCAATCTGATG | |
| | GCAGATGACCCAACCCACCAAAAAGAACTGCTGTCCTTTCCTCTCATCC | |
| | CTGTGACCGGTCCTGACGGGAAGAAGGAACTCCAACACCAGATCCGGA | |
| | CCCAATCCTTGCTCGCCTCAGACAAACAAACTGAACGGTTCATCTTCCT | |
| | CAACACTTACGGATTCATCTATGACACCACACCGGACAAGACAACTTTT | |
| | TCCACCCCAGAGCATATTAATCAGCCTAAGAGGACGACGGTGAGTGCC | |
| | GCGATGATGACCATTGGCCTGGTTCCCGCCAATATACCCCTGAACGAAC | |
| | TAACGGCTACTGTGTTCAGCCTTAAAGTAAGAGTGAGGAAAAGTGCGA | |
| | GGTATCGGGAAGTGGTCTGGTATCAATGCAATCCAGTACCGGCCCTGCT | |
| | TGCAGCCACCAGGTTTGGTCGCCAAGGAGGTCTCGAGTCGAGCACTGGA | |
| | GTCAGTGTAAAGGCTCCCGAGAAGATAGATTGTGAGAAGGATTATACCT | |
| | ACTACCCTTATTTCTTATCTGTGTGCTACATCGCCACCTCCAACCTGTTC | |
| | AAGGTACCGAGGATGGTTGCTAATGCAACCAACAGTCAATTATACCACC | |
| | TTACCATGCAGGTCACATTTGCCTTTCCAAAAAACATCCCTCCAGCCAA | |
| | CCAGAAACTCCTGACACAGGTGGATGAGGGATTCGAGGGCACTGTGGA | |
| | TTGCCATTTTGGGAACATGCTGAAAAAGGATCGGAAAGGGAACATGAG | |
| | GACACTGTCCCAGGCGGCAGATAAGGTCAGACGAATGAATATTCTTGTT | |
| | GGTATCTTTGACTTGCATGGGCCAACGCTCTTCCTGGAGTATACCGGGA | |
| | AACTGACAAAGGCTCTGCTAGGGTTCATGTCCACCAGCCGAACAGCAAT | |
| | CATCCCCATATCTCAGCTCAATCCCATGCTGAGTCAACTCATGTGGAGC | |
| | AGTGATGCCCAGATAGTAAAGTTAAGGGTTGTCATAACTACATCCAAAC | |
| | GCGGCCCGTGCGGGGGTGAGCAGGAGTATGTGCTGGATCCCAAATTCA | |
| | CAGTTAAGAAAGAAAAGGCTCGACTCAACCCTTTCGAGAAGGCAGCCT | |
| | AATGATTTAATCCGCAAGATCCCAGAAATCAGACCACTCTATACTATCC | |
| | ACTGATCACTGGAAATGTAATTGTACAGTTGATGAATCTGTGAAGAATC | |
| | AATTAAAAAACCGGATCCTTATTAGGGTGGGGAAGTAGTTGATTGGGTG | |
| | TCTAAACAAAAGCATTTCTTCACACCTCCCCGCCACGAAACAACCACAA | |
| | TGAGGCTATCAAACACAATCTTGACCTTGATTCTCATCATACTTACCGGC | |
| | TATTTGATAGGTGTCCACTCCACCGATGTGAATGAGAAACCAAAGTCCG | |
| | AAGGGATTAGGGGTGATCTTACACCAGGTGCGGGTATTTTCGTAACTCA | |
| | AGTCCGACAGCTCCAGATCTACCAACAGTCTGGGTACCATGATCTTGTC | |
| | ATCAGATTGTTACCTCTTCTACCAACAGAGCTTAATGATTGTCAAAGGG | |
| | AAGTTGTCACAGAGTACAATAACACTGTATCACAGCTGTTGCAGCCTAT | |
| | CAAAACCAACCTGGATACTTTGTTGGCAGATGGTAGCACAAGGGATGTT | |
| | GATATACAGCCGCGATTCATTGGGGCAATAATAGCCACAGGTGCCCTGG | |
| | CTGTAGCAACGGTAGCTGAGGTAACTGCAGCTCAAGCACTATCTCAGTC | |
| | AAAAACGAATGCTCAAAATATTCTCAAGTTGAGAGATAGTATTCAGGCC | |
| | ACCAACCAAGCAGTTTTTGAAATTTCACAGGGACTCGAAGCAACTGCAA | |
| | CCGTGCTATCAAAACTGCAAACTGAGCTCAATGAGAATATCATCCCAAG | |
| | TCTGAACAACTTGTCCTGTGCTGCCATGGGGAATCGCCTTGGTGTATCA | |
| | CTCTCACTCTATTTGACCTTAATGACCACTCTATTTGGGGACCAGATCAC | |
| | AAACCCAGTGCTGACGCCAATCTCTTACAGCACCCTATCGGCAATGGCG | |
| | GGTGGTCACATTGGTCCAGTGATGAGTAAGATATTAGCCGGATCTGTCA | |
| | CAAGTCAGTTGGGGGCAGAACAACTGATTGCTAGTGGCTTAATACAGTC | |
| | ACAGGTAGTAGGTTATGATTCCCAGTATCAGCTGTTGGTTATCAGGGTC | |
| | AACCTTGTACGGATTCAGGAAGTCCAGAATACTAGGGTTGTATCACTAA | |
| | GAACACTAGCAGTCAATAGGGATGGTGGACTTTACAGAGCCCAGGTGC | |
| | CACCCGAGGTAGTTGAGCGATCTGGCATTGCAGAGCGGTTTTATGCAGA | |
| | TGATTGTGTTCTAACTACAACTGATTACATCTGCTCATCGATCCGATCTT | |
| | CTCGGCTTAATCCAGAGTTAGTCAAGTGTCTCAGTGGGGCACTTGATTC | |
| | ATGCACATTTGAGAGGGAAAGTGCATTACTGTCAACTCCCTTCTTTGTAT | |
| | ACAACAAGGCAGTCGTCGCAAATTGTAAAGCAGCGACATGTAGATGTA | |
| | ATAAACCGCCATCTATCATTGCCCAATACTCTGCATCAGCTCTAGTAAC | |
| | CATCACCACCGACACTTGTGCTGACCTTGAAATTGAGGGTTATCGTTTC | |
| | AACATACAGACTGAATCCAACTCATGGGTTGCACCAAACTTCACGGTCT | |
| | CAACCTCACAAATAGTATCGGTTGATCCAATAGACATATCCTCTGACAT | |
| | TGCCAAAATTAACAATTCTATCGAGGCTGCGCGAGAGCAGCTGGAACTG | |
| | AGCAACCAGATCCTTTCCCGAATCAACCCACGGATTGTGAACGACGAAT | |
| | CACTAATAGCTATTATCGTGACAATTGTTGTGCTTAGTCTCCTTGTAATT | |
| | GGTCTTATTATTGTTCTCGGTGTGATGTACAAGAATCTTAAGAAAGTCC | |
| | AACGAGCTCAAGCTGCTATGATGATGCAGCAAATGAGCTCATCACAGCC | |
| | TGTGACCACCAAATTGGGGACACCCTTCTAGGTGAATAATCATATCAAT | |
| | CCATTCAATAATGAGCGGGACATACCAATCACCAACGACTGTGTCACAA | |
| | GGCCGGTTAGGAATGCACCGGATCTCTCTCCTTCCTTTTTAATTAAAAAC | |
| | GGTTGAACTGAGGGTGAGGGGGGGGGTGTGCATGGTAGGGTGGGGAAG | |
| | GTAGCCAATTCCTGCCCATTGGGCCGACCGTACCAAGAGAAGTCAACAG | |
| | AAGTATAGATGCAGGGCGACATGGAGGGTAGCCGTGATAACCTCACAG | |
| | TAGATGATGAATTAAAGACAACATGGAGGTTAGCTTATAGAGTTGTATC | |
| | CCTCCTATTGATGGTGAGTGCCTTGATAATCTCTATAGTAATCCTGACGA | |
| | GAGATAACAGCCAAAGCATAATCACGGCGATCAACCAGTCGTATGACG | |
| | CAGACTCAAAGTGGCAAACAGGGATAGAAGGGAAAATCACCTCAATCA | |
| | TGACTGATACGCTCGATACCAGGAATGCAGCTCTTCTCCACATTCCACT | |
| | CCAGCTCAATACACTTGAGGCAAACCTGTTGTCCGCCCTCGGAGGTTAC | |
| | ACGGGAATTGGCCCCGGAGATCTAGAGCACTGTCGTTATCCGGTTCATG | |
| | ACTCCGCTTACCTGCATGGAGTCAATCGATTACTCATCAATCAAACAGC | |
| | TGACTACACAGCAGAAGGCCCCCTGGATCATGTGAACTTCATTCCGGCA | |
| | CCAGTTACGACTACTGGATGCACAAGGATCCCATCCTTTTCTGTATCATC | |
| | ATCCATTTGGTGCTATACACACAATGTGATTGAAACAGGTTGCAATGAC | |
| | CACTCAGGTAGTAATCAATATATCAGTATGGGGGTGATTAAGAGGGCTG | |
| | GCAACGGCTTACCTTACTTCTCAACAGTCGTGAGTAAGTATCTGACCGA | |
| | TGGGTTGAATAGAAAAAGCTGTTCCGTAGCTGCGGGATCCGGGCATTGT | |
| | TACCTCCTTTGTAGCCTAGTGTCAGAGCCCGAACCTGATGACTATGTGTC | |
| | ACCAGATCCCACACCGATGAGGTTAGGGGTGCTAACAAGGGATGGGTC | |
| | TTACACTGAACAGGTGGTACCCGAAAGAATATTTAAGAACATATGGAG | |
| | CGCAAACTACCCTGGGGTAGGGTCAGGTGCTATAGCAGGAAATAAGGT | |
| | GTTATTCCCATTTTACGGCGGAGTGAAGAATGGATCAACCCCTGAGGTG | |
| | ATGAATAGGGGAAGATATTACTACATCCAGGATCCAAATGACTATTGCC | |
| | CTGACCCGCTGCAAGATCAGATCTTAAGGGCAGAACAATCGTATTATCC | |
| | TACTCGATTTGGTAGGAGGATGGTAATGCAGGGAGTCCTAACATGTCCA | |
| | GTATCCAACAATTCAACAATAGCCAGCCAATGCCAATCTTACTATTTCA | |
| | ACAACTCATTAGGATTCATCGGGGCGGAATCTAGGATCTATTACCTCAA | |
| | TGGTAACATTTACCTTTATCAAAGAAGCTCGAGCTGGTGGCCTCACCCC | |
| | CAAATTTACCTACTTGATTCCAGGATTGCAAGTCCGGGTACGCAGAACA | |
| | TTGACTCAGGCGTTAACCTCAAGATGTTAAATGTTACTGTCATTACACG | |
| | ACCATCATCTGGCTTTTGTAATAGTCAGTCAAGATGCCCTAATGACTGCT | |
| | TATTCGGGGTTTATTCAGATGTCTGGCCTCTTAGCCTTACCTCAGACAGC | |
| | ATATTTGCATTTACAATGTACTTACAAGGGAAGACGACACGTATTGACC | |
| | CAGCTTGGGCGCTATTCTCCAATCATGTAATTGGGCATGAGGCTCGTTT | |
| | GTTCAACAAGGAGGTTAGTGCTGCTTATTCTACCACCACTTGTTTTTCGG | |
| | ACACCATCCAAAACCAGGTGTATTGTCTGAGTATACTTGAAGTCAGAAG | |
| | TGAGCTCTTGGGGGCATTCAAGATAGTGCCATTCCTCTATCGTGTCTTAT | |
| | AGGCACCTGCTTGGTCAAGAACCCTGAGCAGCCATAAAATTAACACTTG | |
| | ATCTTCCTTAAAAACACCTATCTAAATTACTGTCTGAGATCCCTGATTAG | |
| | TTACCCTTTCAATCAATCAATTAATTTTTAATTAAAAACGGAAAAATGG | |
| | GCCTAGTTCCAAGGAAAGGATGGGACCCATTAGGGTGGGGAAGGATTA | |
| | CTTTGTTCCTTGACTCGCACCCACGTACACCCAATCCCATTCCTGTCAAG | |
| | AAGGAACCCTTCCCAAACTCACCTTGCAATGTCCAATCAGGCAGCTGAG | |
| | ATTATACTACCCACCTTCCATCTTTTATCACCCTTGATCGAGAATAAGTG | |
| | CTTCTACTACATGCAATTACTTGGTCTCGTGTTACCACATGATCACTGGA | |
| | GATGGAGGGCATTCGTCAATTTTACAGTGGATCAAGCACACCTTAAAAA | |
| | TCGTAATCCCCGCTTAATGGCCCACATCGATCACACTAAGGATAGACTA | |
| | AGGGCTCATGGTGTCTTGGGTTTCCACCAGACTCAGACAAGTGAGAGCC | |
| | GTTTCCGTGTCTTGCTCCATCCTGAAACTTTACCTTGGCTATCAGCAATG | |
| | GGAGGATGCATCAACCAGGTTCCCAAGGCATGGCGGAACACTCTGAAA | |
| | TCTATCGAGCACAGTGTGAAGCAGGAGGCGACTCAACTGAAGTTACTCA | |
| | TGGAAAAAACCTCACTAAAGCTAACAGGAGTATCTTACTTATTCTCCAA | |
| | TTGCAATCCCGGGAAAACTGCAGCGGGAACTATGCCCGTACTAAGTGA | |
| | GATGGCATCAGAACTCTTGTCAAATCCCATCTCCCAATTCCAATCAACA | |
| | TGGGGGTGTGCTGCTTCAGGGTGGCACCATGTAGTCAGCATCATGAGGC | |
| | TCCAACAGTATCAAAGAAGGACAGGTAAGGAAGAGAAAGCAATCACTG | |
| | AAGTTCAGTATGGCTCGGACACCTGTCTCATTAATGCAGACTACACCGT | |
| | CGTTTTTTCCGCACAGGACCGTGTCATAGCAGTCTTGCCTTTCGATGTTG | |
| | TCCTCATGATGCAAGACCTGCTTGAATCCCGACGGAATGTCTTGTTCTGT | |
| | GCCCGCTTTATGTATCCCAGAAGCCAACTACATGAGAGGATAAGTACAA | |
| | TACTGGCCCTTGGAGACCAACTCGGGAGAAAAGCACCCCAAGTCCTGTA | |
| | TGATTTCGTAGCTACCCTCGAATCATTTGCATACGCTGCTGTCCAACTTC | |
| | ATGACAACAACCCTATCTACGGTGGGGCTTTCTTTGAGTTCAATATCCA | |
| | AGAACTGGAAGCTATTTTGTCCCCTGCACTTAATAAGGATCAAGTCAAC | |
| | TTCTACATAAGTCAAGTTGTCTCAGCATACAGTAACCTTCCCCCATCTGA | |
| | ATCAGCAGAATTGCTATGCTTACTACGCCTGTGGGGTCATCCCTTGCTA | |
| | AACAGTCTTGATGCAGCAAAGAAAGTCAGAGAATCTATGTGTGCTGGG | |
| | AAGGTTCTTGATTATAATGCTATTCGACTAGTTTTGTCTTTTTATCATAC | |
| | GTTATTAATCAATGGGTATCGGAAGAAACATAAGGGTCGCTGGCCAAAT | |
| | GTGAATCAACATTCACTACTCAACCCGATAGTGAAGCAGCTTTACTTTG | |
| | ATCAGGAGGAGATCCCACACTCTGTTGCCCTTGAGCACTATTTAGATAT | |
| | CTCGATGATAGAATTTGAGAAGACTTTTGAAGTGGAACTATCTGATAGT | |
| | CTAAGCATCTTTCTGAAGGATAAGTCGATAGCTTTGGATAAACAAGAAT | |
| | GGCACAGTGGTTTTGTCTCAGAAGTGACTCCAAAGCACCTACGAATGTC | |
| | TCGTCATGATCGCAAGTCTACCAATAGGCTATTGTTAGCCTTTATTAACT | |
| | CCCCTGAATTCGATGTTAAGGAAGAGCTTAAATATTTGACTACAGGTGA | |
| | GTATGCCACTGACCCAAATTTCAATGTCTCTTACTCACTGAAAGAGAAG | |
| | GAAGTTAAGAAAGAAGGGCGCATTTTCGCAAAGATGTCACAGAAAATG | |
| | AGAGCATGCCAGGTTATTTGTGAAGAGTTACTAGCACATCATGTGGCTC | |
| | CTTTGTTTAAAGAGAATGGTGTTACACAATCGGAGCTATCCCTGACAAA | |
| | GAATTTGTTGGCTATTAGCCAACTGAGTTACAACTCGATGGCCGCTAAG | |
| | GTGCGATTGCTGAGGCCAGGGGACAAGTTCACCGCTGCACACTATATGA | |
| | CCACAGACCTAAAAAAGTACTGCCTTAACTGGCGGCACCAGTCAGTCAA | |
| | ATTGTTCGCCAGAAGCCTGGATCGACTATTTGGGTTAGACCATGCTTTTT | |
| | CTTGGATACACGTCCGTCTCACCAATAGCACTATGTACGTTGCTGACCC | |
| | ATTCAATCCACCAGACTCAGATGCATGCACAAATTTAGACGACAATAAG | |
| | AACACTGGGATTTTTATTATAAGTGCTCGAGGTGGTATAGAAGGCCTTC | |
| | AACAGAAACTATGGACTGGCATATCAATTGCAATCGCCCAGGCGGCAG | |
| | CAGCCCTCGAGGGCTTACGAATTGCTGCCACTTTGCAGGGGGATAACCA | |
| | GGTTTTAGCGATTACGAAAGAATTCATGACCCCAGTCTCGGAGGATGTA | |
| | ATCCACGAGCAGCTATCTGAAGCGATGTCGCGATACAAGAGGACTTTCA | |
| | CATACCTTAATTATTTAATGGGGCACCAATTGAAGGATAAAGAAACCAT | |
| | CCAATCCAGTGACTTCTTCGTTTACTCCAAAAGGATCTTCTTCAATGGGT | |
| | CAATCCTAAGTCAATGCCTCAAGAACTTCAGTAAACTCACTACCAATGC | |
| | CACTACCCTTGCTGAGAACACTGTAGCCGGCTGCAGTGACATCTCCTCA | |
| | TGCATAGCCCGTTGTGTGGAAAACGGGTTGCCTAAGGATGCTGCATATG | |
| | TTCAGAATATAATCATGACTCGGCTTCAACTGTTGCTAGATCACTACTAT | |
| | TCTATGCATGGTGGCATAAACTCAGAGTTAGAGCAGCCAACTCTAAGTA | |
| | TCCCTGTCCGAAACGCAACCTATTTACCATCTCAATTAGGCGGTTACAA | |
| | TCATTTGAATATGACCCGACTATTCTGTCGCAATATCGGTGACCCGCTTA | |
| | CTAGTTCTTGGGCAGAGTCAAAAAGACTAATGGATGTTGGCCTTCTCAG | |
| | TCGTAAGTTCTTAGAGGGGATATTATGGAGACCCCCGGGAAGTGGGAC | |
| | ATTTTCAACACTCATGCTTGATCCGTTCGCACTTAACATTGATTACTTAA | |
| | GGCCACCAGAGACAATAATCCGAAAACACACCCAAAAAGTCTTGTTGC | |
| | AGGATTGTCCTAATCCTCTATTAGCAGGTGTAGTTGACCCGAACTACAA | |
| | CCAGGAATTAGAATTATTAGCTCAGTTCCTGCTTGATCGGGAAACCGTT | |
| | ATTCCCAGGGCTGCCCATGCCATCTTTGAACTGTCTGTCTTGGGAAGGA | |
| | AAAAACATATACAAGGATTGGTTGATACTACAAAAACAATTATTCAGTG | |
| | CTCATTAGAAAGACAGCCACTGTCCTGGAGGAAAGTTGAGAACATTGTA | |
| | ACCTACAATGCGCAGTATTTCCTCGGGGCCACCCAGCAGGTTGACACCA | |
| | ATATCTCAGAAAGGCAGTGGGTGATGCCAGGTAATTTCAAGAAGCTTGT | |
| | ATCTCTTGACGATTGCTCAGTCACGTTGTCCACTGTGTCACGGCGCATTT | |
| | CTTGGGCCAATCTACTTAACTGGAGGGCTATAGATGGTTTGGAAACTCC | |
| | AGATGTGATAGAGAGTATTGATGGCCGCCTTGTGCAATCATCCAATCAA | |
| | TGCGGCCTATGTAATCAAGGATTGGGCTCCTACTCCTGGTTCTTCTTGCC | |
| | CTCCGGGTGTGTGTTCGACCGTCCACAAGATTCTCGAGTGGTTCCAAAG | |
| | ATGCCATACGTGGGATCCAAAACGGATGAGAGACAGACTGCGTCAGTG | |
| | CAGGCTATACAGGGATCCACATGTCACCTTAGAGCAGCATTGAGACTTG | |
| | TATCACTCTACCTTTGGGCCTATGGAGATTCTGACATATCATGGCTAGA | |
| | AGCCGCGACATTGGCTCAAACACGGTGCAATATTTCTCTTGATGACCTG | |
| | CGGATCCTGAGCCCTCTTCCTTCCTCGGCAAATTTACACCACAGATTGA | |
| | ATGACGGGGTAACACAAGTGAAATTCATGCCCGCCACATCGAGCCGGG | |
| | TGTCAAAGTTCGTCCAAATTTGCAATGACAACCAGAATCTTATCCGTGA | |
| | TGATGGGAGTGTTGATTCCAATATGATTTATCAGCAGGTTATGATATTA | |
| | GGGCTTGGAGAGATTGAATGTTTGTTAGCTGACCCAATCGATACAAACC | |
| | CAGAACAACTGATTCTTCACCTACACTCTGATAATTCTTGCTGTCTCCGG | |
| | GAGATGCCAACGACCGGTTTTGTACCTGCTTTAGGATTGACCCCATGCT | |
| | TAACTGTCCCAAAGCACAATCCGTATATTTATGATGATAGCCCAATACC | |
| | CGGTGATTTGGATCAGAGGCTCATTCAAACCAAATTCTTTATGGGTTCT | |
| | GACAATCTAGATAATCTTGATATCTACCAGCAGCGAGCTTTACTGAGTC | |
| | GGTGTGTGGCTTATGACATTATCCAATCAGTATTCGCTTGCGATGCACC | |
| | AGTATCTCAGAAGAATGATGCAATCCTTCACACTGACTACCATGAAAAT | |
| | TGGATCTCAGAGTTCCGATGGGGTGACCCTCGCATAATCCAAGTAACAG | |
| | CAGGTTACGAGTTAATTCTGTTCCTTGCATACCAGCTTTATTATCTCAGA | |
| | GTGAGGGGTGACCGTGCAATCCTGTGTTATATTGATAGGATACTCAACA | |
| | GGATGGTATCTTCCAATCTAGGCAGTCTCATCCAGACGCTCTCTCATCCG | |
| | GAGATTAGGAGGAGATTTTCATTGAGTGATCAAGGGTTCCTTGTCGAAA | |
| | GGGAGCTAGAGCCAGGTAAGCCACTGGTAAAACAAGCGGTTATGTTCC | |
| | TAAGGGACTCAGTCCGCTGCGCTTTAGCAACTATCAAGGCAGGAATTGA | |
| | GCCTGAGATCTCCCGAGGTGGCTGTACCCAGGATGAGCTGAGCTTTACC | |
| | CTTAAGCACTTACTATGTCGGCGTCTCTGTATAATTGCTCTCATGCATTC | |
| | GGAAGCAAAGAACTTGGTCAAAGTTAGAAACCTTCCAGTAGAGGAAAA | |
| | AACCGCCTTACTATACCAGATGTTGATCACTGAGGCCAATGCCAGGAGA | |
| | TCAGGGTCTGCTAGTATCATCATAAGCTTAGTTTCAGCACCCCAGTGGG | |
| | ACATTCATACACCAGCGTTGTATTTTGTATCAAAGAAAATGCTGGGGAT | |
| | GCTCAAAAGGTCAACCACACCCTTGGATATAAGTGACCTTTCTGAGAGC | |
| | CAGAACCTCACACCAACAGAATTGAATGATGTTCCTGGTCACATGGCAG | |
| | AGGAATTTCCCTGTTTGTTTAGCAGTTATAACGCTACATATGAAGACAC | |
| | AATTACTTACAATCCAATGACTGAAAAACTCGCAGTGCACTTGGACAAT | |
| | GGTTCCACCCCTTCCAGAGCGCTTGGTCGTCACTACATCCTGCGACCCCT | |
| | TGGGCTTTACTCGTCTGCATGGTACCGGTCTGCAGCACTATTAGCGTCA | |
| | GGGGCCCTCAGTGGGTTGCCTGAGGGGTCAAGCCTGTACTTGGGAGAG | |
| | GGGTATGGGACCACCATGACTCTACTTGAGCCCGTTGTCAAGTCCTCAA | |
| | CTGTTTACTACCATACATTGTTTGACCCAACCCGGAATCCTTCACAGCGG | |
| | AACTACAAACCAGAACCGCGGGTATTCACTGATTCCATTTGGTACAAGG | |
| | ATGATTTCACACGACCACCTGGTGGCATTGTAAATCTATGGGGTGAAGA | |
| | CGTACGTCAGAGTGATATTACACAGAAAGACACGGTTAATTTCATATTA | |
| | TCTCGGGTCCCGCCAAAATCACTCAAATTGATACACGTTGATATTGAGT | |
| | TCTCCCCAGACTCTGATGTACGGACGCTACTATCTGGCTATTCCCATTGT | |
| | GCACTATTGGCCTACTGGCTACTGCAACCTGGAGGGCGATTTGCGGTTA | |
| | GAGTTTTCTTAAGTGACCATATCATAGTCAACTTGGTCACTGCCATTCTG | |
| | TCCGCTTTTGACTCTAATCTGGTGTGCATTGCGTCAGGATTGACACACAA | |
| | GGATGATGGGGCAGGTTATATTTGTGCAAAGAAGCTTGCAAATGTTGAG | |
| | GCTTCAAGAATTGAGTATTACTTGAGGATGGTCCACGGCTGTGTTGACT | |
| | CATTAAAAATTCCTCATCAATTAGGAATCATTAAATGGGCTGAGGGTGA | |
| | AGTGTCCCGACTTACCAAAAAGGCGGATGATGAAATAAACTGGCGGTT | |
| | AGGTGATCCAGTTACCAGATCATTTGATCCGGTTTCTGAGCTAATAATT | |
| | GCGCGAACAGGGGGATCAGTATTAATGGAATACGGGACTTTTACTAACC | |
| | TCAGGTGTGCGAACTTGGCAGATACATATAAACTTTTGGCTTCAATTGT | |
| | AGAGACCACCTTAATGGAAATAAGGGTTGAGCAAGATCAGTTGGAAGA | |
| | TGATTCGAGGAGACAAATCCAGGTAGTCCCTGCTTTTAATACAAGATCC | |
| | GGGGGAAGGATCCGTACATTGATTGAGTGTGCTCAGCTGCAGGTCATAG | |
| | ATGTTATCTGTGTGAACATAGATCACCTCTTTCCCAAACACCGACATGCT | |
| | CTTGTCACACAACTTACTTACCAGTCAGTGTGCCTTGGGGACTTGATTGA | |
| | AGGCCCCCAAATTAAGACATATCTAAGGGCCAGGAAGTGGATCCAACG | |
| | TAGGGGACTCAATGAGACAATTAACCATATCATCACTGGACAAGTGTCG | |
| | CGGAATAAGGCAAGGGATTTTTTCAAGAGGCGCCTGAAGTTGGTTGGCT | |
| | TTTCGCTCTGTGGCGGTTGGGGCTACCTCTCACTTTAGCTGCTTAGATTG | |
| | TTGATTATTATGAATAATCGGAGTCGAAATCGTAAATAGAAAGACATAA | |
| | AATTGCAAATAAGCAATGATCGTATTAATATTTAATAAAAAATATGTCT | |
| | TTTATTTCGT | |
| Avian | ACGAAAAAGAAGAATAAAAGGCAGAAGCCTTTTAAAAGGAACCCTGGG | 79 |
| paramyxovirus | CTGTCGTAGGTGTGGGAAGGTTGTATTCCGAGTGCGCCTCCGAGGCATC | |
| 4 isolate | TACTCTACACCTATCACAATGGCTGGTGTCTTCTCCCAGTATGAGAGGTT | |
| Uria_aalge/ | TGTGGATAACCAATCCCAAGTGTCAAGGAAGGATCATCGGTCCCTGGCA | |
| Russia/Tyule- | GGGGGATGCCTCAAAGTCAACATCCCTATGCTTGTCACTGCATCTGAAG | |
| niy_Island/l | ATCCCACCACTCGTTGGCAACTAGCATGTTTATCTTTAAGGCTCTTGATC | |
| 15/2015, | TCCAACTCATCAACCAGCGCTATCCGCCAGGGGGCAATACTGACTCTCA | |
| genome | TGTCACTACCATCACAAAATATGAGAGCAACGGCAGCTATTGCTGGTTC | |
| Genbank: | CACAAATGCAGCTGTTATCAACACTATGGAAGTCCTAAGTGTCAACGAC | |
| KU601399.1 | TGGACCCCATCCTTCGACCCTAGGAGCGGTCTCTCTGAAGAGGATGCTC | |
| | AGGTTTTTAGAGACATGGCAAGGGATCTGCCCCCTCAGTTCACCTCCGG | |
| | ATCACCCTTTACATCAGCTTTGGCGGAGGGGTTTACCCCAGAAGACACC | |
| | CACGACCTAATGGAGGCCTTGACCAGTGTGCTGATACAGATCTGGATCC | |
| | TGGTGGCTAAGGCCATGACCAACATTGATGGTTCTGGGGAGGCCAATGA | |
| | GAGACGTCTTGCAAAGTATATCCAGAAGGGACAGCTCAATCGCCAGTTT | |
| | GCAATTGGTAATCCTGCTCGTCTAATAATCCAACAGACGATCAAAAGCT | |
| | CCTTAACTGTCCGCAGGTTCTTGGTCTCTGAGCTTCGTGCATCACGAGGT | |
| | GCGGTGAAAGAAGGATCCCCTTATTATGCAGCTGTTGGGGATATCCACG | |
| | CATACATCTTTAACGCAGGACTGACACCATTCTTGACTACTTTAAGATAT | |
| | GGGATCGGCACCAAGTATGCTGCTGTTGCACTCAGTGTGTTCGCTGCAG | |
| | ACATTGCAAAATTAAAGAGTCTACTTACCTTATACCAAGATAAGGGTGT | |
| | GGAGGCCGGATACATGGCACTCCTTGAAGATCCAGACTCCATGCACTTT | |
| | GCACCTGGAAACTTCCCACACATGTACTCCTACGCGATGGGGGTGGCTT | |
| | CTTACCATGACCCCAGCATGCGCCAGTACCAATATGCCAGGAGGTTCCT | |
| | CAGCCGACCCTTCTACTTGCTAGGAAGGGACATGGCCGCCAAGAATACA | |
| | GGCACGCTGGATGAGCAACTGGCAAAGGAACTGCAAGTGTCAGAGAGA | |
| | GACCGCGCCGCACTGTCCGCTGCGATTCAATCAGCAATGGAAGGGGGA | |
| | GAATCCGACGACTTCCCACTGTCGGGATCCATGCCGGCTCTCTCCGACA | |
| | ATGCACAACCAGTTACCCCAAGAACCCAACAGTCCCAGCTCTCCCCTCC | |
| | CCAATCATCAAGCATGTCTCAATCAGCGCCCAGGACCCCGGACTACCAG | |
| | CCTGATTTTGAACTGTAGGCTGCATCCATGCACCAGCAGCAGGCCAAAG | |
| | AAACCACCCTCCTCTCCACACATCCCACCCAATCACCCGCTGAGACTCA | |
| | ATCCAACACCCTAGCATCCCCCTCATTTAATTAAAAACTGACCAATAGG | |
| | GTGGGGAAGGAGAGTTATTGGCTATTGCCAAGTTCGTGCAGCAATGGAT | |
| | TTTACCGATATTGATGCTGTCAACTCATTAATCGAATCATCATCAGCAAT | |
| | CATAGATTCCATACAGCATGGAGGGCTGCAACCATCAGGCACTGTCGGC | |
| | CTATCGCAAATCCCAAAGGGGATAACCAGCGCTTTAACCAAAGCCTGG | |
| | GAGGCTGAGGCAGCAAATGCTGGCAATGGGGACACCCAACAAAAGTCT | |
| | GACAGTCTGGAGGATCATCAGGCCAACGACACAGACTCCCCCGAAGAC | |
| | ACAGGCACTAACCAGACCATCCAGGAAACCAATATCGTTGAAACACCC | |
| | CACCCCGAAGTGCTATCGGCAGCCAAAGCCAGACTCAAGAGGCCCAAG | |
| | GCAGGGAAGGACACCCACGACAATCCCTCTGCGCAACCTGATCATCTTT | |
| | TAAAGGGGGGCCCCTTGAGCCCACAACCAGTGGCACCGTGGGTGCAAA | |
| | ATCCGCCCATTCATGGAGGTCCCGGCACCGCCGATCCCCGCCCATCACA | |
| | AACTCAGGATCATTCCCTCACCGGAGAGAGATGGCAATCGTCACCGACA | |
| | AAGCAACCGGAGCCATCGAACTGGTGGAATGGTGCAACCCGGGGTGCA | |
| | CAGCAATCCGAATTGAACCTACCAGACTCGACTGTGTATGCGGACACTG | |
| | CCCCACCATCTGCAGCCTCTGCATGTATGACGACTGATCAGGTACAACT | |
| | ATTAATGAAGGAGGTTGCCGATATGAAATCACTCCTTCAGGCACTAGTG | |
| | AGGAACCTAGCTGTCCTGCCTCAACTAAGGAACGAGGTTGCAGCAATCA | |
| | GGACATCACAGGCTATGATAGAGGGGACACTTAATTCAATCAAGATTCT | |
| | CGACCCTGGGAATTATCAGGAATCATCACTAAACAGTTGGTTCAAACCA | |
| | CGACAAGATCACGCGGTTGTTGTGTCCGGACCAGGGAATCCATTGACCA | |
| | TGCCAACCCCAATCCAGGACAATACCATATTCCTGGATGAATTGGCAAG | |
| | ACCTCATCCTAGTTTGGTCAATCCGTCCCCGCCCACTACCAACACTAATG | |
| | TTGATCTTGGCCCACAGAAGCAGGCTGCGATAGCTTATATCTCAGCAAA | |
| | ATGCAAGGATCAAGGGAAACGAGATCAGCTCTCAAAGCTCATCGAGCG | |
| | AGCAACCACCTTGAGTGAGATCAACAAAGTTAAAAGACAGGCTCTTGG | |
| | CCTCTAGATCACCCAATCACCCCCAGTAATGAGTACAACAATAATCAGA | |
| | ACCTCCCTAAACCACATGGCCAACCAAGCACACCATCCACACCACCCCT | |
| | TACTATCCTTTGCCAGAAACTCCGCCGCAGCTGATTTATTCAAAAGAAG | |
| | CCACTTGGTATAACCTAGCAACCGCAAGATAGGGTGGGGAAGGTGCTTT | |
| | GCCTGCAAGAGGGCTCCCTCATCTTCAGACACTTACCCGCCAACCCACC | |
| | AGTGACACAATGGCAGACATGGACACTGTATATATCAATCTGATGGCAG | |
| | ATGATCCAACCCACCAAAAAGAACTGCTGTCCTTTCCCCTCATTCCAGT | |
| | GACTGGTCCCGACGGGAAAAAGGAACTCCAACACCAGGTTCGGACTCA | |
| | ATCCTTGCTCGCCTCAGACAAGCAAACTGAGAGGTTCATCTTCCTCAAC | |
| | ACTTACGGGTTTATCTATGACACTACACCGGACAAGACAACTTTTTCCA | |
| | CCCCAGAGCATATCAATCAGCCCAAGAGAACGATGGTGAGTGCTGCAA | |
| | TGATGACCATCGGCCTGGTCCCCGCCAATATACCCTTGAACGAACTAAC | |
| | AGCTACTGTGTTTGGCCTGAAGGTGAGAGTGAGGAAGAGTGCGAGATA | |
| | TCGAGAGGTGGTCTGGTATCAGTGCAACCCTGTACCAGCCCTGCTGGCA | |
| | GCCACCAGGTTCGGTCGCCAAGGGGGTCTCGAATCGAGCACTGGAGTC | |
| | AGTGTGAAGGCCCCTGAGAAGATAGATTGTGAGAAGGATTATACTTACT | |
| | ACCCTTATTTCCTATCTGTGTGCTACATCGCTACTTCCAACCTGTTCAAG | |
| | GTACCAAAAATGGTTGCTAATGCGACCAACAGTCAATTATACCATCTGA | |
| | CCATGCAGGTCACATTTGCCTTTCCAAAAAACATCCCCCCAGCTAACCA | |
| | GAAACTCCTGACACAAGTGGATGAAGGATTCGAGGGCACTGTGGACTG | |
| | CCATTTTGGGAACATGCTGAAAAAGGATCGGAAAGGGAATATGAGGAC | |
| | ATTGTCGCAGGCGGCAGATAAGGTCAGACGGATGAACATCCTTGTTGGT | |
| | ATCTTTGACTTGCATGGGCCGACACTCTTCCTGGAGTATACCGGGAAAC | |
| | TAACAAAAGCTCTGCTAGGGTTCATGTCTACCAGCCGAACAGCAATCAT | |
| | CCCCATATCTCAGCTCAATCCTATGCTGAGTCAACTCATGTGGAGTAGT | |
| | GATGCCCAGATAGTAAAATTAAGAGTGGTCATAACTACATCCAAACGC | |
| | GGCCCATGCGGGGGTGAGCAGGAGTATGTGCTGGATCCCAAATTCACA | |
| | GTTAAAAAAGAAAAAGCCCGACTCAATCCTTTCAAGAAGGCAGCCCAA | |
| | TGATCAAATCTGCAGGATCTCAGAAATCAGACCACTCTATACTATCCAC | |
| | TGATTAATAGACACGTAGCTATACAGTTGATGAACCTATGAAGAATCAA | |
| | TTAGCAAACCGAATCCTTGCTAGGGTGGGGAAGGAGTTGATTGGGTGTC | |
| | TAAACAAAAGCACTCCTTTGCACCTCCTCGCCACGAAACAACCATAATG | |
| | AGGTTATCACGCACAATCCTGGCCCTGATTCTAGGCACACTTACCGGCT | |
| | ATTTAATGGATGCCCACTCCACCACTGTGAACGAGAGACCAAAGTCTGA | |
| | AGGGATTAGGGGTGATCTTATACCAGGCGCAGGTATCTTTGTAACTCAA | |
| | GTCCGACAACTACAGATCTACCAACAGTCTGGGTATCATGACCTTGTCA | |
| | TCAGGTTATTACCTCTTCTACCGGCAGAACTCAATGATTGTCAAAGGGA | |
| | AGTTGTCACAGAGTACAACAATACGGTATCACAGCTGTTGCAGCCTATC | |
| | AAAACCAACCTGGATACCTTATTGGCTGATGGTGGTACAAGGGATGCCG | |
| | ATATACAGCCGCGGTTCATTGGGGCGATAATAGCCACAGGTGCCCTGGC | |
| | GGTGGCTACGGTAGCTGAGGTGACTGCAGCCCAAGCACTATCGCAGTCG | |
| | AAAACGAACGCTCAAAATATTCTCAAGTTGAGAGATAGTATTCAGGCCA | |
| | CCAACCAGGCAGTTTTTGAAATTTCACAAGGACTTGAGGCAACTGCAAC | |
| | TGTGCTATCAAAACTGCAAACTGAGCTCAATGAGAACATTATCCCAAGC | |
| | CTGAACAACTTGTCCTGTGCTGCTATGGGGAATCGCCTTGGTGTATCACT | |
| | ATCACTCTACTTGACCTTAATGACCACCCTATTTGGGGACCAGATCACA | |
| | AACCCAGTGCTGACACCAATCTCCTATAGCACTCTATCGGCAATGGCAG | |
| | GTGGTCACATTGGCCCGGTGATGAGTAAGATATTAGCCGGATCTGTCAC | |
| | AAGTCAGTTGGGGGCAGAACAGTTGATTGCTAGCGGCTTAATACAGTCA | |
| | CAAGTAGTGGGTTATGATTCCCAATATCAATTATTGGTTATCAGGGTCA | |
| | ATCTTGTACGGATTCAAGAGGTCCAGAATACGAGGGTCGTATCACTAAG | |
| | AACACTAGCGGTCAATAGGGATGGTGGACTTTATAGAGCCCAGGTGCCT | |
| | CCTGAGGTAGTTGAACGGTCTGGCATTGCAGAGCGATTTTACGCAGATG | |
| | ATTGCGTTCTTACTACAACTGATTACATTTGCTCATCGATCCGATCTTCT | |
| | CGGCTTAATCCAGAGTTAGTCAAGTGTCTCAGTGGGGCACTTGATTCAT | |
| | GCACATTTGAGAGGGAAAGTGCATTATTGTCAACCCCTTTCTTTGTATAC | |
| | AACAAGGCAGTTGTCGCAAATTGTAAAGCAGCAACATGTAGATGTAAT | |
| | AAACCGCCGTCTATTATTGCCCAATACTCTGCATCGGCTCTGGTCACCAT | |
| | CACCACTGACACCTGCGCCGACCTTGAAATTGAGGGTTATCGCTTCAAC | |
| | ATACAGACTGAATCCAACTCATGGGTTGCACCAAACTTCACTGTCTCGA | |
| | CTTCACAGATTGTATCAGTTGATCCAATAGACATCTCCTCTGACATTGCC | |
| | AAAATCAACAGTTCCATCGAGGCTGCAAGAGAGCAGCTGGAACTAAGC | |
| | AACCAGATCCTCTCCCGGATTAACCCACGAATCGTGAATGATGAATCAC | |
| | TGATAGCTATTATCGTGACAATTGTTGTGCTTAGTCTCCTCGTAATCGGT | |
| | CTGATTGTTGTTCTCGGTGTGATGTATAAGAATCTTAAGAAAGTCCAAC | |
| | GAGCTCAAGCTGCCATGATGATGAAGCAAATGAGCTCATCACAGCCTGT | |
| | GACCACTAAATTAGGGACGCCTTTCTAGGAGGATAATCATATTACTCTA | |
| | CTCAATGATGAGCAAGACGTACCAATTATCAATGATTGTGTCACAAGGC | |
| | CGGTTGGGAATGCACCGAATCTCTCCCCTTTCTTTTTAATTAAAAACATT | |
| | TGAAGTGAGGATAAGAGGGGGGAAGAGTATGGTAGGGTGGGGAAGGT | |
| | AGCCAATCCCTGCCTATTAGGCTGATCGTATCAAAAGAACCCAACAGAA | |
| | GTCTAGATACAGGGCAACATGGAGGGCAGCCGTGATAATCTAACAGTG | |
| | GATGATGAATTAAAGACAACATGGAGGTTAGCTTATAGAGTTGTGTCCC | |
| | TCCTATTGATGGTGAGCGCTTTGATAATCTCTATAGTAATCCTGACAAG | |
| | AGATAACAGCCAAAGCATAATCACGGCGATCAACCAGTCATCTGACGC | |
| | AGACTCTAAGTGGCAAACGGGAATAGAAGGGAAAATCACCTCCATTAT | |
| | GACTGATACGCTCGATACCAGAAATGCAGCCCTTCTCCACATTCCACTC | |
| | CAGCTCAACACGCTTGCGGCGAACCTATTGTCCGCCCTTGGAGGCAACA | |
| | CAGGAATTGGCCCCGGAGATCTGGAACACTGCCGTTACCCTGTTCATGA | |
| | CACCGCTTACCTGCATGGAGTTAATCGATTACTCATCAACCAGACAGCT | |
| | GATTATACAGCAGAAGGCCCCCTAGATCATGTGAACTTCATACCAGCCC | |
| | CGGTTACGACCACTGGATGCACAAGGATACCATCCTTTTCTGTGTCATC | |
| | GTCCATTTGGTGCTATACACACAACGTGATTGAAACCGGTTGCAATGAC | |
| | CACTCAGGTAGTAACCAATATATCAGCATGGGAGTCATTAAGAGAGCA | |
| | GGCAACGGCTTACCTTACTTCTCAACAGTTGTAAGTAAGTATCTGACTG | |
| | ATGGGTTGAATAGGAAGAGCTGTTCTGTAGCTGCCGGATCTGGGCATTG | |
| | CTACCTCCTTTGCAGCTTAGTGTCGGAGCCTGAACCTGATGACTATGTAT | |
| | CACCTGATCCCACACCGATGAGGTTAGGGGTGCTAACGTGGGATGGGTC | |
| | TTACACTGAACAGGTGGTACCCGAAAGAATATTCAAGAACATATGGAG | |
| | TGCAAACTACCCGGGAGTAGGGTCAGGTGCTATAGTAGGAAATAAAGT | |
| | GTTATTCCCATTTTACGGCGGAGTGAGGAATGGATCGACCCCGGAGGTG | |
| | ATGAATAGGGGAAGATACTACTACATCCAGGATCCAAATGACTATTGCC | |
| | CTGACCCGCTGCAAGATCAGATCTTAAGAGCGGAACAATCGTATTACCC | |
| | AACTCGATTCGGTAGGAGGATGGTAATGCAAGGGGTCCTAGCATGTCCA | |
| | GTATCCAACAATTCAACAATAGCAAGCCAATGTCAATCTTACTATTTTA | |
| | ATAACTCATTAGGGTTCATCGGGGCAGAATCTAGAATCTATTATCTCAA | |
| | TGGTAACATTTATCTTTATCAGAGAAGCTCGAGTTGGTGGCCTCACCCC | |
| | CAAATCTACCTGCTTGATTCTAGAATTGCAAGTCCGGGTACTCAGACCA | |
| | TTGACTCAGGTGTCAATCTCAAAATGTTAAATGTCACTGTGATTACACG | |
| | ACCATCATCTGGTTTTTGTAATAGTCAGTCACGATGCCCTAATGATTGCT | |
| | TATTCGGGGTCTATTCGGATATCTGGCCTCTTAGCCTTACCTCAGATAGC | |
| | ATATTCGCATTCACAATGTATTTACAGGGGAAGACAACACGTATTGACC | |
| | CGGCTTGGGCGCTATTCTCCAATCATGCAATTGGGCATGAGGCTCGTCT | |
| | GTTTAATAAGGAAGTTAGTGCTGCTTATTCTACCACCACTTGTTTTTCGG | |
| | ACACCATCCAAAATCAGGTGTATTGCCTGAGTATACTTGAGGTCAGAAG | |
| | TGAGCTCTTGGGAGCATTCAAAATAGTACCATTCCTCTACCGCGTCTTGT | |
| | AGGCATCCATTCAGCCAAAAAACTTGAGTGACCATGAGATTGACACCTG | |
| | ATCCCCCTCAAAGACACCTATCTAAATTACTGTTCTAGACCCATGATTA | |
| | GGTACCTTCTTAATCAATCATTTGGTTTTTAATTAAAAATGGAAAAATG | |
| | GACCTAGTTCCAAGAGAGGGCTGGAACCCATTAGGGTGGGGAAGGATT | |
| | GCTTTGCTCCTTGACTCACACTCACGTACACTCGATCAGACTTCTGTTAA | |
| | AAAGGAAACCTTCTCAAACTCGCCCCACGATGTCCAATCAGGCAGCTGA | |
| | GATTATACTACCTAGCTTCCATCTAGAATCACCCTTAATCGAGAATAAG | |
| | TGCTTCTATTATATGCAATTACTTGGTCTCGTGTTGCCACATGATCACTG | |
| | GAGATGGAGGGCATTCGTTAACTTTACAGTGGATCAGGTGCACCTTAAA | |
| | AATCGTAATCCCCGCTTAATGGCCCACATCGACTACACTAAAGATAGAT | |
| | TGAGGACTCATGGTGTCTTAGGTTTCCACCAGACTCAGACAAGTTTGAG | |
| | CCGTTATCGTGTTTTGCTCCATCCTGAAACCTTACCTTGGCTGTCAGCCA | |
| | TGGGAGGATGCATCAATCAGGTGCCTAAAGCATGGCGGAACACCCTGA | |
| | AATCGATCGAGCACAGTGTAAAGCAGGAGGCACCTCAACTAAAGCTAC | |
| | TCATGGAGAGAACCTCATTAAAATTAACTGGGGTACCTTACTTGTTCTCT | |
| | AATTGCAATCCCGGGAAAACCAAAGCAGGAACTATACCTGTCCTAAGT | |
| | GAGATGGCATCGGAACTCTTGTCAAATCCTATCTCCCAATTCCAATCAA | |
| | CATGGGGATGTGCTGCTTCGGGGTGGCACCATGTAGTCAGTATCATGAG | |
| | GCTTCAGCAATATCAAAGAAGGACAGGTAAGGAGGAAAAAGCAATCAC | |
| | TGAAGTTCAGTATGGCACAGACACCTGTCTCATTAACGCAGACTACACC | |
| | GTTGTTTTTTCCACACAGAACCGTATCATAACGGTCTTGCCTTTCGATGT | |
| | TGTCCTCATGATGCAAGACCTGCTCGAATCCCGACGGAATGTCCTGTTC | |
| | TGTGCCCGCTTTATGTATCCCAGAAGCCAACTTCATGAGAGGATAAGTA | |
| | CAATATTAGCCCTTGGAGACCAATTGGGGAGGAAAGCACCCCAAGTCCT | |
| | GTATGATTTTGTAGCAACCCTTGAGTCATTTGCATACGCAGCGGTTCAA | |
| | CTTCATGACAACAATCCTACCTACGGTGGGGCCTTCTTTGAATTCAACAT | |
| | CCAAGAGTTAGAATCGATTCTGTCCCCTGCACTTAGTAAGGATCAGGTC | |
| | AACTTCTACATAAGTCAAGTTGTCTCAGCGTACAGTAACCTTCCTCCATC | |
| | CGAATCGGCAGAGCTGCTGTGCCTGTTACGCCTGTGGGGTCATCCCTTG | |
| | CTAAACAGCCTTGATGCAGCAAAGAAAGTCAGGGAGTCTATGTGCGCC | |
| | GGGAAGGTTCTCGATTACAACGCCATTCGACTTGTCTTGTCTTTTTATCA | |
| | TACGTTGCTAATCAATGGGTACCGGAAGAAACACAAGGGTCGCTGGCC | |
| | AAATGTGAATCAACATTCACTTCTCAACCCGATAGTGAGGCAGCTTTAT | |
| | TTTGATCAGGAGGAGATCCCACACTCTGTTGCCCTTGAGCACTATTTGG | |
| | ATGTTTCAATGATAGAATTTGAAAAAACTTTTGAAGTGGAACTATCTGA | |
| | CAGCCTAAGCATCTTCCTGAAGGATAAGTCGATAGCTTTGGATAAGCAA | |
| | GAATGGTATAGTGGTTTTGTCTCAGAAGTGACTCCGAAGCACCTGCGAA | |
| | TGTCCCGTCATGATCGCAAGTCTACCAATAGGCTCCTGTTAGCCTTCATT | |
| | AACTCCCCTGAATTCGATGTTAAGGAAGAGCTTAAATACTTGACTACGG | |
| | GTGAGTACGCCACTGACCCAAATTTCAATGTCTCATACTCACTTAAAGA | |
| | GAAGGAGGTAAAGAAAGAAGGGCGCATTTTCGCAAAAATGTCACAAAA | |
| | GATGAGAGCGTGCCAGGTTATTTGTGAAGAATTGCTAGCACATCATGTG | |
| | GCTCCTTTGTTTAAAGAGAATGGTGTTACTCAATCAGAGCTATCCCTGA | |
| | CAAAAAATTTGTTGGCTATTAGCCAACTGAGTTACAACTCGATGGCCGC | |
| | TAAGGTTCGATTGCTGCGGCCAGGGGACAAGTTCACTGCTGCACACTAT | |
| | ATGACCACAGACCTAAAAAAGTACTGTCTTAATTGGCGGCACCAGTCAG | |
| | TCAAACTGTTCGCCAGAAGCCTGGATCGACTGTTTGGGTTAGACCATGC | |
| | TTTTTCTTGGATACATGTCCGTCTCACCAACAGCACTATGTACGTTGCTG | |
| | ACCCCTTTAATCCACCAGACTCAGATGCATGCACAAATTTAGACGACAA | |
| | TAAGAATACCGGGATCTTTATTATAAGTGCACGAGGTGGTATAGAAGGC | |
| | CTCCAACAAAAGCTATGGACTGGCATATCAATTGCAATTGCCCAAGCGG | |
| | CAGCGGCCCTCGAAGGCTTACGAATTGCTGCTACTCTGCAGGGGGATAA | |
| | CCAAGTTTTGGCGATTACAAAGGAATTCATGACCCCAGTCCCAGAAGAT | |
| | GTAATCCATGAGCAGCTATCTGAGGCGATGTCTCGATACAAAAGGACTT | |
| | TCACATACCTCAATTATTTAATGGGACATCAGTTGAAGGATAAGGAAAC | |
| | CATCCAATCTAGTGATTTCTTTGTTTACTCCAAAAGAATCTTCTTCAATG | |
| | GATCAATCTTAAGTCAATGCCTCAAGAACTTCAGTAAACTCACTACTAA | |
| | TGCCACTACCCTTGCTGAGAATACTGTGGCCGGCTGCAGTGACATCTCT | |
| | TCATGCATTGCCCGTTGTGTGGAAAACGGGTTGCCAAAGGATGCCGCAT | |
| | ACATCCAGAATATAATCATGACTCGGCTTCAACTATTGCTAGATCATTA | |
| | CTATTCAATGCATGGCGGCATAAACTCAGAGTTAGAGCAGCCAACGTTA | |
| | AGTATCTCTGTTCGAAACGCAACCTACTTACCATCTCAACTAGGCGGTT | |
| | ACAATCATTTAAATATGACTCGACTATTCTGCCGCAATATCGGCGACCC | |
| | GCTTACCAGTTCTTGGGCAGAGTCAAAAAGACTAATGGATGTTGGTCTC | |
| | CTCAGTCGTAAGTTCTTGGAGGGGATATTATGGAGACCCCCGGGAAGTG | |
| | GGACGTTTTCAACACTCATGCTTGATCCGTTCGCACTTAACATTGATTAC | |
| | CTGAGGCCGCCAGAGACAATTATCCGAAAACACACCCAAAAAGTCTTA | |
| | TTGCAAGATTGTCCAAACCCCCTATTAGCAGGTGTCGTTGACCCAAACT | |
| | ACAACCAAGAATTAGAGCTGTTAGCTCAGTTCTTGCTTGATCGGGAAAC | |
| | CGTTATTCCCAGGGCTGCCCATGCCATCTTTGAGTTGTCTGTCTTGGGGA | |
| | GGAAAAAACATATACAAGGATTGGTAGATACTACAAAAACAATTATTC | |
| | AGTGCTCATTGGAAAGACAGCCATTGTCCTGGAGGAAAGTTGAGAACA | |
| | TTGTTACCTACAACGCGCAGTATTTCCTCGGGGCCACCCAACAGGCTGA | |
| | CACTAATGTCTCAGAAGGGCAGTGGGTGATGCCAGGTAACTTCAAGAA | |
| | GCTTGTGTCCCTTGACGATTGCTCGGTCACGTTGTCTACCGTATCACGGC | |
| | GCATATCGTGGGCCAATCTACTGAACTGGAGAGCTATAGACGGTTTGGA | |
| | AACCCCGGATGTGATAGAGAGTATCGATGGCCGCCTTGTACAATCATCC | |
| | AATCAATGTGGCCTATGTAATCAAGGGTTGGGGTCCTACTCCTGGTTCTT | |
| | CTTGCCCTCTGGGTGTGTGTTCGACCGTCCACAAGATTCCCGGGTGGTTC | |
| | CAAAGATGCCATATGTGGGGTCCAAAACAGATGAGAGACAGACTGCAT | |
| | CAGTGCAAGCTATACAAGGATCCACTTGTCACCTCAGGGCGGCATTGAG | |
| | GCTTGTATCACTCTACCTATGGGCCTATGGGGATTCTGACATATCATGGC | |
| | TAGAAGCTGCGACACTGGCTCAAACACGGTGCAACGTTTCTCTTGATGA | |
| | CTTGCGAATCTTGAGCCCTCTCCCTTCTTCGGCGAATTTACACCACAGAT | |
| | TAAATGACGGGGTAACACAGGTTAAATTCATGCCCGCCACATCGAGCCG | |
| | AGTGTCAAAGTTCGTCCAAATTTGCAATGACAACCAGAATCTTATCCGT | |
| | GACGATGGAAGTGTTGATTCCAATATGATTTATCAACAGGTTATGATAT | |
| | TAGGGCTTGGGGAGATTGAATGCTTGTTAGCTGACCCAATTGATACAAA | |
| | CCCAGAACAATTGATTCTTCATCTACACTCTGATAATTCTTGCTGTCTCC | |
| | GGGAGATGCCAACGACCGGCTTTGTACCAGCTCTAGGACTGACCCCATG | |
| | TTTAACTGTCCCAAAGCACAATCCTTACATATATGATGATAGCCCAATA | |
| | CCTGGTGATTTGGATCAGAGGCTCATTCAGACCAAATTTTTCATGGGTTC | |
| | TGACAATTTGGATAATCTTGATATCTACCAACAGCGAGCTTTACTGAGT | |
| | AGGTGTGTGGCTTATGATGTTATCCAATCGATCTTTGCTTGTGATGCACC | |
| | AGTCTCTCAGAAGAATGACGCAATCCTTCACACTGACTATCATGAGAAT | |
| | TGGATCTCAGAGTTCCGATGGGGTGACCCTCGTATTATCCAAGTAACGG | |
| | CAGGCTACGAGTTAATTCTGTTCCTTGCATACCAGCTTTATTATCTCAGA | |
| | GTGAGAGGTGATCGTGCAATCCTGTGTTATGTTGACAGGATACTCAATA | |
| | GGATGGTATCTTCCAATCTAGGCAGTCTCATCCAGACACTCTCTCATCCA | |
| | GAGATTAGGAGGAGATTCTCGTTGAGTGATCAAGGGTTCCTTGTTGAGA | |
| | GGGAACTAGAGCCAAGTAAGCCCTTGGTTAAACAAGCGGTTATGTTCTT | |
| | GAGGGACTCAGTCCGCTGCGCTCTAGCTACTATCAAGGCAGGAATTGAG | |
| | CCTGAGATCTCCCGAGGTGGCTGTACTCAGGATGAGCTAAGCTTTACTC | |
| | TTAAGCACTTACTGTGTCGGCGTCTCTGTGTAATCGCTCTCATGCATTCA | |
| | GAGGCAAAGAACTTGGTTAAGGTTAGAAACCTTCCTGTAGAAGAGAAA | |
| | ACCGCCTTACTGTATCAGATGTTGGTCACTGAGGCCAATGCTAGGAAAT | |
| | CAGGATCTGCTAGCATTATCATAAACCTAGTATCGGCACCCCAGTGGGA | |
| | TATTCATACACCAGCATTGTATTTTGTGTCAAAGAAAATGTTAGGGATG | |
| | CTTAAGAGGTCAACCACACCCTTGGATATAAGTGACCTCTCTGAGAGCC | |
| | AGAATCCCGCACCGGCAGAGCTGAATGATGTTCCTGATCACATGGCAGA | |
| | AGAATTTCCCTGTTTGTTTAGTAGTTATAACGCTACATATGAAGACACA | |
| | ATCACTTACAATCCAATGACTGAAAAACTCGCCTTGCACTTGGACAATA | |
| | GTTCCACCCCATCCAGAGCACTTGGTCGTCACTACATCCTGCGGCCTCTT | |
| | GGGCTTTACTCATCTGCATGGTACCGGTCTGCAGCACTACTAGCATCAG | |
| | GGGCCCTAAATGGGTTGCCTGAGGGGTCAAGCCTGTATCTAGGAGAAG | |
| | GGTACGGGACCACCATGACTCTGCTTGAGCCCGTTGTCAAGTCTTCAAC | |
| | TGTTTACTACCACACATTGTTTGACCCAACCCGGAATCCTTCACAGCGG | |
| | AACTATAAACCAGAACCACGGGTATTCACGGATTCTATTTGGTACAAGG | |
| | ATGATTTCACACGGCCACCTGGTGGTATTATCAACCTGTGGGGTGAAGA | |
| | TATACGTCAGAGTGATATCACACAGAAAGACACGGTCAACTTCATACTA | |
| | TCTCAGATCCCGCCAAAGTCACTTAAGTTGATACACGTTGATATTGAAT | |
| | TCTCACCAGACTCCGATGTACGGACACTACTTTCTGGCTATTCTCATTGT | |
| | GCATTATTGGCCTACTGGCTATTGCAACCTGGAGGGCGATTTGCGGTTA | |
| | GGGTTTTCTTAAGTGACCATGTCATAGTAAACTTGGTCACTGCAATTCTG | |
| | TCTGCTTTTGACTCTAATTTGGTGTGCATTGCATCAGGATTGACACACAA | |
| | GGATGATGGGGCAGGTTATATTTGCGCAAAGAAGCTTGCAAATGTTGAG | |
| | GCTTCAAGGATTGAATACTACCTGAGGATGGTCCATGGTTGTGTTGACT | |
| | CATTAAAGATCCCTCATCAATTAGGAATCATTAAATGGGCCGAGGGTGA | |
| | GGTGTCCCAACTTACCAGAAAGGCAGATGATGAAATAAATTGGCGGTT | |
| | AGGTGATCCGGTTACCAGATCATTTGATCCAGTTTCTGAGCTAATCATTG | |
| | CACGAACAGGGGGGTCTGTATTGATGGAATACGGGGCTTTTACTAACCT | |
| | CAGGTGTGCGAACTTGGCAGATACATACAAACTTCTGGCTTCAATTGTA | |
| | GAGACCACCTTAATGGAAATAAGGGTTGAACAAGACCAGTTGGAAGAT | |
| | AATTCGAGGAGGCAAATCCAAATAGTCCCCGCTTTTAACACGAGATCTG | |
| | GGGGAAGGATCCGTACACTGATTGAGTGTGCTCAGCTGCAGATTATAGA | |
| | TGTTATTTGTGTAAACATAGATCACCTCTTTCCTAGACACCGACATGTTC | |
| | TTGTCACGCAACTTACCTACCAGTCGGTGTGCCTTGGGGACTTGATTGA | |
| | AGGCCCCCAAATTAAGACGTATCTGAGGGCCAGAAAGTGGATCCAACG | |
| | TCGGGGACTCAATGAGACAGTTAACCATATCATCACTGGACAAGTGTCA | |
| | CGGAATAAAGCAAGGGATTTTTTCAAGAGGCGCCTGAAGTTGGTTGGCT | |
| | TTTCACTCTGCGGTGGTTGGAGCTACCTCTCACTTTAACTGTTCAAGTTG | |
| | TTGATTATTATGAATAATCGGAGTCGGAATCGTAAATAGTAAGCCACAA | |
| | AGTCGTGAATAAACAATGATTGCATTAGTATTTAATAAAAAATATGTCT | |
| | TTTATTTCGT | |
| Avian | ACGAAAAAGAAGAATAAAAGGCAGAAGCCTTTTAAAAGGAACCCTGGG | 80 |
| paramyxovirus | CTGTCGTAGGTGTGGGAAGGTTGTATTCCGAGCGCGCCTCCGAGGCATC | |
| 4 isolate | TACTCTACACCTATCACAATGGCTGGTGTCTTCTCCCAATATGAGAGGTT | |
| APMV- | TGTGGACAATCAATCCCAAGTGTCAAGGAAGGATCATCGGTCCCTGGCA | |
| 4/Egyptian | GGGGGATGCCTTAAAGTCAACATTCCTATGCTTGTCACTGCATCTGAAG | |
| goose/South | ATCCCACCACTCGTTGGCAACTAGCGTGTTTATCTTTGAGGCTCTTGATC | |
| Africa/N146 | TCCAACTCATCAACCAGTGCTATCCGCCAGGGGGCAATACTGACTCTCA | |
| 8/2010, | TGTCACTACCATCACAAAATATGAGAGCAACGGCAGCTATTGCTGGTTC | |
| complete | CACAAATGCAGCTGTTATCAACACTATGGAAGTCTTGAGTGTCAATGAC | |
| genome | TGGACCCCATCCTTCGACCCTAGGAGCGGTCTCTCTGAAGAGGATGCTC | |
| Genbank: | AGGTTTTCAGAGACATGGCAAAGGACCTGCCCCCTCAGTTCACCTCCGG | |
| JX133079.1 | ATCACCCTTTACATCAGCATTGGCGGAGGGGTTTACCCCAGAAGACACC | |
| | CACGACCTAATGGAGGCCTTGACTAGTGTGCTGATACAGATCTGGATCC | |
| | TGGTGGCTAAGGCCATGACCAACATTGATGGCTCTGGAGAGGCCAATG | |
| | AGAGACGTCTTGCAAAGTACATCCAGAAGGGACAACTCAATCGCCAGT | |
| | TTGCAATTGGTAATCCTGCTCGTCTGATAATCCAACAGACGATCAAAAG | |
| | CTCCTTAACTGTCCGCAGATTCTTGGTCTCTGAACTTCGTGCATCACGAG | |
| | GTGCGGTGAAAGAAGGATCCCCTTACTATGCAGCTGTTGGGGACATCCA | |
| | CGCTTACATCTTTAACGCAGGACTGACACCATTCTTGACTACCTTAAGAT | |
| | ATGGGATCGGCACCAAGTATGCTGCAGTTGCACTCAGTGTGTTCGCTGC | |
| | AGACATTGCAAAATTAAAGAGCCTACTTACCCTATATCAAGACAAGGGT | |
| | GTGGAGGCTGGATACATGGCACTCCTTGAAGATCCAGACTCCATGCACT | |
| | TTGCACCTGGAAACTTCCCACACATGTACTCCTACGCGATGGGGGTGGC | |
| | TTCTTACCATGACCCCAGCATGCGCCAGTACCAATATGCTAGGAGGTTC | |
| | CTCAGCCGACCTTTCTACTTGCTAGGGAGGGACATGGCCGCCAAGAACA | |
| | CAGGCACGCTGGATGAGCAACTGGCAAAGGAACTGCAAGTGTCAGAAA | |
| | GAGACCGCGCCGCATTGTCCGCTGCGATTCAGTCAGCAATAGAGGGGG | |
| | GAGAATCCGACGACTTCCCACTGTCGGGATCCATGCCGGCTCTCTCCGA | |
| | CAATGCGCAACCAGTTACCCCAAGAACCCAACAGTCCCAGCCCTCCCCT | |
| | CCCCAATCATCAAGCATGTCTCAATCAGCACCCAAGACCCCGGACTACC | |
| | AGCCTGATTTTGAACTGTAGGCTGCATCAGTGCACCAACAGCAGGCCAA | |
| | AGGGACCACCCTCCTCCCCACACATCCCACCCAATCACCCGCTGAGACC | |
| | CAATCCAACACCCCAGCATCCCCCTCATTTAATTAAAAACTGACCAATA | |
| | GGGTGGGGAAGGAGAGCTGTTGGCTATCGCCAAGATCGTGCAGCGATG | |
| | GATTTTACCGATATTGATGCTGTCAACTCATTAATTGAATCATCATCAGC | |
| | AATCATAGATTCCATACAGCATGGAGGGCTGCAACCATCAGGTACTGTT | |
| | GGCCTATCGCAAATCCCCAAGGGGATAACCAGCGCTTTAACCAAGGCCT | |
| | GGGAGGCTGAGACAGCAACTGCTGGCTACGGGGACACCCAACACAAAT | |
| | CTGACAGTCCGGAGGATCATCAGGCCAACGACACAGACTCCCCCGAAG | |
| | ACACAGGCACCAACCAGACCATCCAGGAAGCCAACATCGTCGAAACAC | |
| | CCCACCCCGAAGTTCTATCGGCAGCCAAAGCCAGACTCAAGAGGCCCA | |
| | AGGCAGGGAAGGACACCCACGACAATCCCCCTGCGCAACCCGATCCCC | |
| | TTTTAAAGGGGGGCCCCCTGAGCCCACAACCAGCAGCACCGTGGGTGC | |
| | AAAATTCACCCATTCATGGAGGTCCCGGCACCGCCGATCCCCGCCCATC | |
| | ACAAACTCAGGATCATTCCCTCACCGGAGAGAGATGGCAATCGTCACCG | |
| | ATAAAGCAACCGGAGACATTGAACTGGTGGAATGGTGCAACCCGGGGT | |
| | GCACAGCAATCCGAACTGAACCAACCAGACTCGACTGTGTATGCGGAT | |
| | ACTGCCCCACCATCTGCAGCCTCTGCATGTATGACGACTGATCAGGTAC | |
| | AACTATTAATGAAGGAGGTTGCCGATATGAAATCACTCCTTCAGGCACT | |
| | AGTGAGGAACCTAGCTGTCCTGCCTCAACTAAGGAACGAGGTTGCAGC | |
| | AATCAGGACATCACAGGCTATGATAGAGGGGACACTCAATTCAATCAA | |
| | GATTCTCGACCCTGGGAATTATCAAGAATCATCACTGAACAGTTGGTTC | |
| | AAACCACGCCAAGATCACGCGGTTGCTGTGTCCGGACCAGGGAATCCAT | |
| | TGACCATGCCAACTCCAATCCAAGACAACACCATATTCCTGGATGAACT | |
| | GGCAAGACCTCATCCTAGTTTGGTCAATCCGTCCCCGCCCACTACCAAC | |
| | ACTAATGTTGACCTTGGCCCACAGAAGCAGGCTGCGATAGCTTATATCT | |
| | CAGCAAAATGCAAGGATCAAGGGAGACGAGATCAGCTCTCAAAGCTCA | |
| | TCGAGCGAGCAACCACCTTGAGTGAGATCAACAAAGTCAAAAGACAGG | |
| | CCCTTGGCCTCTAGACCACTCGACCACCCCCAGTAATGAACACAACAAT | |
| | AATCAGAACCTCCCTAAACCACACGGCCAACCCAGCACACCATCCACAC | |
| | CGCCCACCACTATCCCCCGCCAAAAACTCCGCTGCAGCCGATTTATTCA | |
| | AAAGAAGCCACTTGATATGACTTATCAACCGCAAGGTAGGGTGGGGAA | |
| | GGTGCTTTGCCTGCAAGAGGGCTCCCTCATCTTCAGACACGTACCCGCC | |
| | AACCCACCAGTGACGCAATGGCAGACATGGACACTGTATATATCAATCT | |
| | GATGGCAGATGATCCAACCCACCAAAAAGAACTGCTGTCCTTCCCTCTC | |
| | ATTCCAGTGACTGGTCCCGACGGGAAAAAGGAACTCCAACACCAGGTT | |
| | CGGACTCAATCCTTGCTCGCCTCAGACAAGCAAACTGAGAGGTTCATCT | |
| | TCCTCAACACTTACGGGTTTATCTATGACACTACACCGGACAAGACAAC | |
| | TTTTTCCACCCCAGAGCATATCAATCAGCCCAAGAGAACGATGGTGAGT | |
| | GCTGCAATGATGACCATCGGCCTGGTCCCCGCCAATATACCCTTGAACG | |
| | AACTAACAGCTACTGTGTTTGGCCTGAAAGTAAGAGTGAGGAAGAGTG | |
| | CGAGATATCGAGAGGTGGTCTGGTATCAGTGCAACCCTGTACCAGCCCT | |
| | GCTTGCAGCCACCAGGTTTGGTCGCCAAGGAGGTCTCGAATCGAGCACT | |
| | GGAGTCAGTGTGAAGGCCCCCGAGAAGATAGATTGCGAGAAGGATTAT | |
| | ACTTACTACCCTTATTTCCTATCTGTGTGCTACATCGCCACTTCTAACCT | |
| | GTTCAAGGTACCAAAAATGGTTGCTAATGCGACCAACAGTCAATTATAC | |
| | CACCTGACGATGCAGGTCACATTTGCCTTTCCAAAAAACATTCCCCCAG | |
| | CTAACCAGAAACTCCTGACACAAGTGGATGAAGGATTCGAGGGCACTG | |
| | TGGACTGCCATTTTGGGAACATGCTGAAAAAGGATCGGAAAGGGAATA | |
| | TGAGGACATTGTCGCAGGCGGCAGATAAGGTCCGACGGATGAACATCC | |
| | TTGTTGGTATCTTTGACTTGCATGGGCCGACACTCTTCCTGGAGTATACC | |
| | GGGAAACTAACGAAAGCTCTGTTAGGGTTCATGTCTACCAGCCGAACAG | |
| | CAATCATCCCCATATCTCAGCTCAATCCTATGCTGAGTCAACTCATGTGG | |
| | AGCAGTGATGCTCAGATAGTAAAATTAAGAGTGGTCATAACTACATCCA | |
| | AACGCGGCCCATGCGGGGGTGAGCAGGAATATGTGCTGGACCCCAAAT | |
| | TCACAGTTAAAAAAGAAAAAGCCCGACTCAACCCTTTCAAGAAGGCAG | |
| | CTTAATGATCAAATCTGCAGGATCTCAGGAATCAGACCACTCTATACTA | |
| | TCTACTGATCAATAGATATGTAGCTATACAGTTGATGAACCTATGAAGA | |
| | ATCAATTAGCAAACCGAATCCTTGCTAGGGTGGGGAAGGAATTGATTGG | |
| | GTGTCTAAACAAAAGCACTTCTTTGCACCTACTCACCACAAAACAATCA | |
| | TAATGAGGTTATCACGAACAATCCTGGCCCTGATTCTCGGCGCACTTAC | |
| | CGGCTATTTAATGGATGCCCACTCCACCACTGTGAATGAGAGACCAAAG | |
| | TCTGAGGGGATTAGGGGTGACCTTATACCAGGTGCAGGAATCTTTGTAA | |
| | CTCAAATCCGGCAACTACAGATCTACCAACAATCTGGGTATCATGACCT | |
| | TGTCATCAGGTTATTACCTCTTTTACCGGCAGAACTCAATGATTGCCAAA | |
| | GGGAAGTTGTCACAGAGTACAACAATACAGTATCACAGCTGTTGCAGCC | |
| | TATCAAAACTAACCTGGATACCTTATTGGCTGATGGTGGCACAAGGGAT | |
| | GCCGATATACAGCCGCGGTTCATTGGGGCGATAATAGCCACAGGTGCCC | |
| | TGGCAGTGGCTACGGTAGCTGAGGTGACTGCAGCCCAAGCACTATCTCA | |
| | GTCGAAAACGAACGCTCAAAATATTCTCAAGTTGAGAGATAGTATTCAG | |
| | GCCACCAACCAGGCAGTTTTTGAAATTTCACAAGGACTTGAGGCAACTG | |
| | CAACTGTACTATCAAAACTGCAAGCTGAGCTCAATGAGAACATTATCCC | |
| | AAGTCTGAACAACTTGTCCTGTGCTGCCATGGGGAATCGCCTTGGTGTA | |
| | TCACTATCACTCTACTTGACCCTAATGACTACCCTATTTGGGGACCAGAT | |
| | CACAAACCCAGTGCTGACACCAATCTCCTATAGCACTTTATCGGCAATG | |
| | GCAGGTGGTCACATTGGCCCGGTGATGAGTAAAATATTAGCCGGATCTG | |
| | TCACAAGTCAGTTGGGGGCAGAACAGTTGATTGCTAGCGGCTTAATACA | |
| | ATCACAGGTAGTAGGTTATGATTCCCAATATCAATTATTGGTTATCAGG | |
| | GTCAACCTTGTACGGATTCAAGAGGTCCAGAATACGAGGGTCGTATCAC | |
| | TAAGAACACTAGCGGTCAATAGGGATGGTGGACTTTATAGAGCCCAGG | |
| | TGCCTCCCGAGGTAGTCGAACGGTCTGGCATTGCAGAGCGATTTTATGC | |
| | AGATGATTGTGTTCTTACTACAACTGATTACATTTGCTCCTCGATCCGAT | |
| | CTTCTCGGCTTAATCCAGAGTTAGTCAAATGTCTCAGTGGGGCACTTGA | |
| | TTCATGCACATTTGAGAGGGAAAGTGCATTATTGTCAACCCCTTTCTTTG | |
| | TATACAACAAGGCAGTTGTCGCAAATTGTAAAGCGGCAACATGTAGAT | |
| | GCAATAAACCGCCGTCTATTATTGCCCAATACTCTGCATCAGCTCTGGTC | |
| | ACCATCACCACCGACACCTGCGCCGACCTTGAAATTGAGGGCTATCGCT | |
| | TCAATATACAGACTGAATCCAACTCATGGGTTGCACCAAACTTCACTGT | |
| | CTCGACTTCACAGATTGTATCAGTTGATCCAATAGACATCTCCTCTGACA | |
| | TTGCTAAAATCAACAGTTCCATCGAGGCTGCAAGAGAGCAGCTGGAACT | |
| | AAGCAACCAGATCCTTTCCCGAATTAACCCACGAATTGTGAATGATGAA | |
| | TCATTGATAGCTATTATCGTGACAATTGTTGTGCTTAGTCTCCTCGTAAT | |
| | CGGTCTGATTGTTGTTCTCGGTGTGATGTATAAGAATCTTAAAAAAGTC | |
| | CAACGAGCTCAAGCTGCCATGATGATGCAGCAGATGAGCTCATCACAG | |
| | CCCGTGACCACTAAATTAGGGACGCCCTTCTAGGATAATAATCATATCA | |
| | CTCTACTCAATGATGAGCAAGACGTACCAATCATCAATGATTGTGTCAC | |
| | AAGGCCGGTAGGGAATGCACCGAATTTCTCCCCTTTCTTTTTAATTAAA | |
| | AACATTTGTAGTGAGGATGAGAAGGGGAAAATGTTTGGTAGGGTGGGG | |
| | AAGGTAGCCAATTCCTGCCTATTAGGCCGACCGTATCAAAAGAACTCAA | |
| | CAGAAGTCCAGATACAAGGTAACATGGAGGGCAGCCGTGATAATCTTA | |
| | CAGTGGATGATGAATTAAAGACAACGTGGAGGTTAGCTTATAGAGTTGT | |
| | GTCCCTTCTATTGATGGTGAGCGCTTTGATAATCTCTATAGTAATCCTGA | |
| | CGAGAGATAACAGCCAAAGCGTAATCACGGCGATCAACCAGTCATCTG | |
| | AAGCTGACTCCAAGTGGCAAACGGGAATAGAAGGGAAAATCACCTCCA | |
| | TTATGACTGATACGCTCGATACCAGGAATGCAGCCCTTCTCCACATTCC | |
| | ACTCCAGCTCAACTCGCTTGAGGCGAACCTATTGTCCGCCCTTGGGGGC | |
| | AACACAGGAATTGGCCCCGGAGATATAGAGCACTGCCGTTACCCTGTTC | |
| | ATGACACCGCTTACCTGCATGGAGTTAATCGATTACTCATCAACCAGAC | |
| | AGCTGATTATACAGCAGAAGGCCCCCTAGATCATGTGAACTTCATTCCA | |
| | GCCCCGGTTACGACCACTGGATGCACAAGGATACCATCCTTTTCCGTGT | |
| | CATCGTCCATTTGGTGCTATACACACAACGTGATTGAAACCGGTTGCAA | |
| | TGACCACTCAGGTAGTAACCAATATATCAGCATGGGAGTCATTAAGAGA | |
| | GCGGGCAACGGCCTACCTTACTTCTCAACAGTTGTAAGTAAGTATCTGA | |
| | CTGATGGGTTGAATAGGAAAAGCTGTTCTGTAGCTGCCGGATCTGGGCA | |
| | TTGCTACCTCCTTTGCAGCTTGGTGTCGGAGCCCGAATCTGATGACTATG | |
| | TGTCACCTGATCCTACACCGATGAGGTTAGGGGTGCTAACGTGGGATGG | |
| | GTCTTACACTGAGCAGGTGGTACCCGAAAGAATATTCAAGAACATATGG | |
| | AGTGCAAACTACCCAGGAGTAGGGTCAGGTGCTATAGTAGGAAATAAG | |
| | GTGTTATTCCCATTTTACGGCGGAGTGAGTAATGGATCGACCCCGGAGG | |
| | TGATGAATAGGGGAAGATATTACTACATCCAGGATCCAAATGACTATTG | |
| | CCCTGACCCGCTGCAAGATCAGATCTTAAGGGCGGAACAATCGTATTAC | |
| | CCAACTCGATTCGGTAGGAGGATGGTGATGCAAGGGGTCCTAGCATGTC | |
| | CAGTATCCAACAATTCAACAATAGCAAGCCAATGTCAATCTTACTATTT | |
| | TAATAACTCATTAGGGTTCATTGGGGCAGAATCTAGGATCTATTACCTC | |
| | AATGATAACATTTATCTTTACCAGAGAAGCTCGAGCTGGTGGCCTCACC | |
| | CCCAGATTTACCTGCTTGATTCTAGGATTGCAAGTCCGGGTACTCAGAA | |
| | CATTGACTCAGGTGTCAATCTCAAGATGTTAAATGTCACTGTAATTACA | |
| | CGACCATCATCTGGTTTTTGTAATAGTCAGTCACGATGCCCTAATGACTG | |
| | CTTATTCGGGGTCTACTCGGATATCTGGCCTCTTAGCCTTACCTCAGATA | |
| | GCATATTCGCATTCACAATGTATTTACAGGGGAAGACAACACGTATTGA | |
| | CCCGGCTTGGGCGCTATTCTCCAATCATGCGATTGGGCATGAGGCTCGT | |
| | CTGTTTAATAAGAAGGTTAGTGCTGCTTATTCTACCACCACTTGTTTTTC | |
| | GGACACCGTCCAAAATCAGGTGTATTGCCTGAGTATACTTGAGGTCAGG | |
| | AGTGAGCTCTTGGGAGCATTCAAAATAGTACCATTCCTCTATCGCGTCTT | |
| | GTAGGCATCCATTCAGCCAGAAAACTTGAGTGACCATGATATTAACACC | |
| | TGATCCCCCTCAAAGACACCTATCTAAATTACTGTTCTAGACTCATGATT | |
| | AGGTACCTTCTTAATCAATCATTTGGTTTTTAATTAAAAATGAAAAAAT | |
| | AGGCCTAGTTCCAAGAGAGGGCTGGAACCCATTAGGGTGGGGAAGGAT | |
| | TGCTTTGCTCCTTGACTCACACACACGTACACTCGATCAGACTCCTGTTT | |
| | AAAAGGAATCCTTCTCAAACTCGCCCCACGATGTCCAATCAGGCGGCTG | |
| | AGATTATACTACCCACCTTCCATCTAGAATCACCCTTAATCGAAAATAA | |
| | GTGCTTCTATTATATGCAATTACTTGGTCTCGTGTTGCCACATGATCACT | |
| | GGAGATGGAGGGCATTCGTTAACTTTACAGTGGATCAGGTGCACCTTAA | |
| | AAATCGTAATCCCCGCTTGATGGCCCACATCGACTACACTAAGGATAGA | |
| | TTAAGGACTCATGGTGTCTTAGGTTTCCACCAGACTCAGACAAGTTTGA | |
| | GCCGTTATCGTGTTTTGCTCCATCCTGAAACCTTATCTTGGCTATCAGCC | |
| | ATGGGGGGATGCATCAATCAGGTTCCTAAAGCATGGCGGAACACTCTG | |
| | AAATCGATCGAGCACAGTGTAAAGCAGGAGGCACCTCAACTAAAGCTA | |
| | CTCATGGAGAGAACCTCATTAAAATTAACTGGAGTACCTTACTTGTTCT | |
| | CTAATTGCAATCCCGGGAAAACCACAGCAGGTACTATGCCTGTCCTAAG | |
| | TGAGATGGCATCGGAACTCTTGTCGAATCCTATCTCCCAATTCCAATCA | |
| | ACATGGGGGTGTGCTGCTTCGGGGTGGCACCATGTAGTCAGTATCATGA | |
| | GGCTCCAACAATACCAAAGAAGGACAGGTAAAGAAGAGAAAGCGATC | |
| | ACTGAAGTTCAGTATGGCACAGACACCTGTCTCATTAATGCAGACTACA | |
| | CTGTTGTGTTTTCCACACAGAACCGTATCATAACAGTCTTGCCTTTTGAT | |
| | GTTGTCCTCATGATGCAAGACCTGCTCGAATCCCGACGGAATGTCCTGT | |
| | TCTGTGCCCGCTTTATGTATCCCAGAAGCCAACTTCATGAGAGGATAAG | |
| | TACAATATTAGCTCTTGGAGACCAACTGGGGAGAAAAGCACCCCAAGT | |
| | CCTGTATGATTTCGTAGCAACCCTTGAGTCATTTGCATACGCGGCTGTTC | |
| | AACTTCATGACAACAATCCTACCTACGGTGGGGCCTTCTTTGAATTCAA | |
| | TATCCAAGAGTTAGAATCCATTCTGTCCCCTGCACTTAGTAAGGATCAG | |
| | GTCAACTTCTACATAAATCAAGTTGTCTCAGCGTACAGTAACCTTCCCCC | |
| | ATCTGAATCGGCAGAATTGCTGTGCCTGTTACGCCTGTGGGGTCACCCC | |
| | CTGCTAAACAGCCTTGATGCAGCAAAGAAAGTCAGGGAGTCTATGTGC | |
| | GCCGGGAAGGTTCTCGATTACAACGCCATTCGACTTGTCTTGTCTTTTTA | |
| | TCATACGTTGCTAATCAACGGATACCGGAAGAAACACAAGGGTCGCTG | |
| | GCCAAATGTGAATCAACATTCACTCCTCAACCCGATAGTGAGGCAGCTT | |
| | TATTTTGATCAGGAGGAGATCCCACACTCTGTTGCTCTTGAGCACTATTT | |
| | GGACGTCTCAATGGTAGAATTTGAAAAAACTTTTGAAGTGGAATTATCT | |
| | GACAGCCTAAGCATCTTCCTAAAGGATAAGTCGATAGCTTTGGATAAGC | |
| | AAGAGTGGTACAGTGGTTTTGTCTCAGAAGTGACTCCGAAGCACCTGCG | |
| | AATGTCCCGTCATGATCGCAAGTCTACCAATAGGCTCCTGTTAGCCTTC | |
| | ATTAACTCCCCTGAATTCGATGTTAAGGAAGAGCTTAAATACTTGACTA | |
| | CGGGTGAGTACGCCACTGACCCAAATTTCAATGTCTCATACTCACTTAA | |
| | AGAGAAGGAAGTAAAGAAAGAGGGGCGCATTTTCGCAAAAATGTCACA | |
| | AAAGATGAGAGCATGCCAGGTTATTTGTGAAGAATTGCTAGCACATCAT | |
| | GTGGCTCCTTTGTTTAAAGAGAATGGTGTTACTCAATCAGAGCTATCCCT | |
| | GACAAAAAATTTGTTGGCTATTAGCCAACTGAGTTACAACTCGATGGCC | |
| | GCTAAGGTGCGATTGCTGAGACCAGGGGACAAGTTCACTGCTGCACACT | |
| | ATATGACCACAGACCTAAAAAAGTACTGTCTTAATTGGCGGCACCAGTC | |
| | AGTCAAACTGTTCGCCAGAAGCCTGGATCGACTGTTTGGGTTAGACCAT | |
| | GCTTTTTCTTGGATACATGTCCGCCTCACCAACAGCACTATGTACGTTGC | |
| | TGACCCCTTCAATCCACCAGACTCAGATGCATGCATTAATTTAGACGAC | |
| | AATAAGAACACTGGGATTTTTATTATAAGTGCACGAGGTGGTATAGAAG | |
| | GCCTCCAACAAAAACTATGGACTGGCATATCAATTGCAATTGCCCAAGC | |
| | GGCAGCGGCCCTCGAAGGCTTACGAATTGCTGCTACTCTGCAGGGGGAT | |
| | AACCAAGTTTTGGCGATTACAAAGGAATTCATGACCCCAGTCCCAGAGG | |
| | ATGTAATCCATGAGCAGCTATCTGAGGCGATGTCTCGATACAAAAGGAC | |
| | TTTCACATACCTCAATTATTTAATGGGACATCAATTGAAGGATAAGGAA | |
| | ACCATCCAATCCAGTGATTTCTTTGTCTATTCCAAAAGAATCTTCTTCAA | |
| | TGGATCAATCTTAAGTCAATGCCTCAAGAACTTCAGTAAACTCACTACT | |
| | AATGCCACTACCCTTGCTGAGAATACTGTGGCCGGCTGCAGTGACATCT | |
| | CTTCATGCATTGCCCGTTGTGTGGAAAACGGGTTGCCTAAGGATGCCGC | |
| | ATATATCCAGAATATAATCATGACTCGGCTTCAATTATTGCTAGATCATT | |
| | ACTATTCAATGCATGGCGGCATAAACTCAGAATTAGAGCAGCCAACTTT | |
| | AAGTATCTCTGTTCGAAACGCAACCTACTTACCATCTCAACTAGGCGGT | |
| | TACAATCATCTAAATATGACCCGACTATTCTGCCGCAATATCGGCGACC | |
| | CGCTTACCAGTTCTTGGGCGGAGTCAAAAAGACTAATGGATGTTGGTCT | |
| | CCTCAGTCGTAAGTTCTTGGAGGGGATATTATGGAGACCCCCGGGAAGT | |
| | GGGACGTTTTCAACACTCATGCTTGACCCGTTCGCACTTAACATTGATTA | |
| | CCTGAGGCCGCCAGAAACAATTATCCGAAAACACACCCAAAAAGTCTT | |
| | GTTGCAAGATTGCCCAAACCCCCTATTAGCAGGTGTCGTTGACCCAAAC | |
| | TACAACCAAGAATTAGAGCTGTTAGCTCAGTTCTTGCTTGATCGGGAGA | |
| | CCGTTATTCCCAGGGCTGCCCATGCCATCTTTGAGTTGTCTGTCTTGGGG | |
| | AGGAAAAAACATATACAAGGATTGGTGGACACTACAAAAACAATTATT | |
| | CAGTGCTCATTGGAAAGACAGCCATTGTCCTGGAGGAAAGTTGAGAAC | |
| | ATTGTTACCTACAACGCGCAGTATTTCCTCGGGGCCACCCAACAGGCTG | |
| | ATACTAATGTCTCAGAAGGGCAGTGGGTGATGCCAGGTAACTTCAAGA | |
| | AGCTTGTGTCCCTTGACGATTGCTCGGTCACGTTGTCTACTGTATCACGG | |
| | CGCATATCGTGGGCCAATCTACTGAACTGGAGAGCTATAGATGGTTTGG | |
| | AAACCCCGGATGTGATAGAGAGTATTGATGGCCGCCTTGTACAATCATC | |
| | AAATCAATGTGGCCTATGTAATCAAGGGTTGGGGTCCTACTCTTGGTTC | |
| | TTCTTGCCCTCTGGGTGTGTGTTCGACCGTCCACAAGATTCCCGGGTAGT | |
| | TCCAAAGATGCCATACGTGGGGTCCAAAACAGATGAGAGACAGACTGC | |
| | ATCAGTGCAAGCTATACAAGGATCCACTTGTCACCTCAGGGCAGCATTG | |
| | AGGCTTGTATCACTCTACTTATGGGCTTATGGAGATTCTGACATATCATG | |
| | GCTAGAAGCTGCGACACTGGCTCAAACACGGTGCAATGTTTCTCTTGAT | |
| | GACTTGCGAATCTTGAGCCCTCTCCCTTCTTCGGCGAATTTACACCACAG | |
| | ATTAAATGACGGGGTAACACAGGTTAAATTCATGCCCGCCACATCGAGC | |
| | CGAGTGTCAAAGTTCGTCCAAATTTGCAATGACAACCAAAATCTTATCC | |
| | GTGATGATGGGAGTGTTGATTCCAATATGATTTATCAACAGGTTATGAT | |
| | ATTAGGGCTTGGGGAGATTGAATGCTTGTTAGCTGACCCAATTGATACA | |
| | AACCCAGAACAATTGATTCTTCATCTACACTCTGATAATTCTTGCTGTCT | |
| | CCGGGAGATGCCAACGACTGGCTTTGTACCTGCTCTAGGACTGACCCCA | |
| | TGTTTAACTGTCCCAAAGCACAATCCTTACATTTATGATGATAGCCCAAT | |
| | ACCTGGTGATTTGGATCAGAGGCTCATTCAGACCAAATTTTTCATGGGT | |
| | TCTGACAATTTGGATAATCTTGATATCTACCAACAGCGAGCTTTACTGA | |
| | GCAGGTGTGTGGCTTATGATGTTATCCAATCGATCTTTGCCTGTGATGCA | |
| | CCAGTCTCTCAGAAGAATGACGCAATCCTTCACACTGACTATCATGAGA | |
| | ATTGGATCTCAGAGTTCCGATGGGGTGACCCTCGTATTATCCAAGTAAC | |
| | GGCAGGCTACGAGTTAATTCTGTTCCTTGCATACCAGCTTTATTATCTCA | |
| | GAGTGAGGGGTGACCGTGCAATCCTGTGTTATATTGACAGGATACTCAA | |
| | TAGGATGGTATCTTCCAATCTAGGCAGTCTCATCCAGACACTCTCTCATC | |
| | CAGAGATTAGGAGGAGATTCTCATTGAGTGATCAAGGGTTCCTTGTTGA | |
| | AAGGGAATTAGAGCCAGGTAAGCCCTTGGTTAAGCAAGCGGTTATGTTC | |
| | TTGAGGGACTCGGTCCGCTGCGCTTTAGCAACTATCAAGGCAGGAATTG | |
| | AGCCTGAGATCTCCCGAGGTGGCTGTACTCAGGATGAGCTGAGCTTTAC | |
| | TCTTAAGCACTTACTATGCCGGCGTCTCTGTGTAATCGCTCTCATGCATT | |
| | CAGAAGCAAAGAACTTGGTTAAAGTCAGAAACCTTCCTGTAGAGGAGA | |
| | AAACCGCCTTACTGTACCAAATGTTGGTCACTGAGGCCAATGCTAGGAA | |
| | GTCAGGATCTGCTAGCATTATCATAAACCTAGTCTCGGCACCCCAGTGG | |
| | GACATTCATACACCAGCACTGTATTTTGTGTCAAAGAAAATGCTAGGGA | |
| | TGCTTAAGAGGTCAACCACACCCTTGGATATAAGTGACCTCTCCGAGAG | |
| | CCAGAATTCCGCACCTGCAGAGCTGACTGATGTTCCTGGTCACATGGCA | |
| | GAAGAGTTTCCCTGTTTGTTTAGTAGTTATAACGCCACATATGAAGACA | |
| | CAATTACTTACAATCCAACGACTGAAAAACTCGCCTTGCACTTGGACAA | |
| | CAGTTCCACCCCATCCAGAGCACTTGGCCGTCACTACATCCTGCGGCCT | |
| | CTTGGGCTTTATTCATCCGCATGGTACCGGTCTGCAGCACTACTAGCGTC | |
| | AGGGGCCTTGAATGGGTTGCCTGAGGGGTCAAGCCTGTATCTAGGAGA | |
| | AGGGTACGGGACCACCATGACTCTGCTTGAGCCCGTTGTCAAGTCTTCA | |
| | ACTGTTTACTACCATACATTGTTTGACCCAACCCGGAATCCTTCTCAGCG | |
| | GAACTATAAGCCAGAACCACGGGTATTCACGGATTCTATTTGGTACAAG | |
| | GATGATTTCACACGGCCACCTGGTGGTATTATCAACCTGTGGGGTGAAG | |
| | ATATACGGCAGAGTGATATCACACAGAAAGACACGGTCAACTTCATACT | |
| | ATCTCAGATCCCGCCAAAATCACTTAAGTTGATACACGTTGATATTGAA | |
| | TTCTCACCAGACTCCGATGTACGGACACTACTATCTGGCTATTCTCATTG | |
| | TGCACTATTAGCCTACTGGCTATTGCAACCTGGAGGGCGATTTGCAGTT | |
| | AGGGTTTTCTTAAGTGACCATATCATAGTAAACTTAGTCACTGCAATTCT | |
| | GTCTGCTTTTGACTCTAATTTGGTGTGCATTGCATCAGGATTGACACACA | |
| | AGGATGATGGGGCAGGTTATATTTGCGCAAAGAAGCTTGCAAATGTTGA | |
| | GGCTTCAAGGATTGAGCACTACTTGAGGATGGTCCATGGTTGCGTTGAC | |
| | TCATTAAAGATCCCTCATCAATTAGGAATCATTAAATGGGCCGAGGGTG | |
| | AGGTGTCCCAACTTACCAGAAAGGCGGATGATGAAATAAATTGGCGGT | |
| | TAGGCGATCCTGTTACCAGATCATTTGATCCAGTTTCTGAGCTAATCATT | |
| | GCACGAACAGGGGGGTCTGTATTAATGGAATACGGGGCTTTTACTAACC | |
| | TCAGGTGTGCGAACTTGGCAGATACATACAAGCTTCTGGCTTCAATTGT | |
| | AGAGACCACCCTAATGGAAATAAGGGTTGAGCAAGATCAGTTGGAAGA | |
| | TAATTCGAGGAGACAAATCCAAGTAGTCCCCGCTTTCAACACGAGATCT | |
| | GGGGGAAGGATCCGTACGCTGATTGAGTGTGCTCAGCTGCAGATTATAG | |
| | ATGTTATTTGTGTAAACATAGACCACCTCTTTCCTAAACACCGACATGTT | |
| | CTTGTCACGCAACTTACCTACCAGTCGGTGTGCCTTGGGGACCTGATTG | |
| | AAGGCCCCCAAATTAAGACGTATCTAAGGGCCAGAAAGTGGATCCAAC | |
| | GTCAGGGACTCAATGAGACAGTTAACCATATCATCACTGGACAAGTGTC | |
| | ACGGAATAAAGCAAGGGATTTTTTCAAGAGGCGCTTGAAGTTGGTTGGG | |
| | TTTTCACTCTGCGGTGGTTGGAGCTACCTCTCACTTTAGCTGTTCAGGTT | |
| | GTCGATTATTATGAATAATCGGAGTCGGAATCGCAAATAGGAAGCCAC | |
| | AAAGTTGTGGAGAAACAATGATTGCATTAGTATTTAATAAAAAATATGT | |
| | CTTTTATTTCGT | |
| Avian | ACGAAAAAGAAGAATAAAAGGCAGAAGCCTTTTAAAAGGAACCCTGGG | 81 |
| paramyxovirus | CTGTCGTAGGTGTGGGAAGGTTGTATTCCGAGTGCGCCTTCGAGGCATC | |
| 4 strain | TACTCTACACCTATCACAATGGCTGGTGTCTTCTCCCAGTATGAGAGGTT | |
| APMV4/duck/ | TGTGGACAATCAATCCCAAGTGTCAAGGAAGGATCATCGTTCCCTGGCA | |
| China/G302/ | GGGGGATGCCTAAAAGTCAACATCCCTATGCTTGTCACTGCATCTGAAG | |
| 2012, | ATCCCACCACTCGTTGGCAACTAGCATGTTTATCCTTAAGGCTCTTGGTC | |
| complete | TCCAACTCATCAACCAGTGCTATCCGCCAGGGGGCGATACTGACTCTCA | |
| genome | TGTCACTACCATCACAAAATATGAGAGCAACGGCAGCTATTGCTGGTTC | |
| Genbank: | CACAAATGCGGCTGTTATCAACACTATGGAAGTCTTGAGTGTCAACGAC | |
| KC439346.1 | TGGACCCCATCCTTCGACCCCAGGAGCGGTCTCTCTGAAGAGGATGCTC | |
| | AGGTTTTCAGAGACATGGCAAGGGACCTGCCCCCTCAGTTCACCTCCGG | |
| | GTCACCCTTTACATCGGCATTGGCGGAGGGGTTTACCCCGGAGGACACC | |
| | CACGACCTAATGGAGGCCCTGACCAGTGTGCTGATACAGATCTGGATCC | |
| | TGGTGGCTAAGGCCATGACCAACATTGATGGCTCTGGGGAAGCCAATG | |
| | AGAGACGTCTTGCAAAGTACATCCAGAAGGGACAGCTTAATCGCCAGTT | |
| | TGCAATTGGTAATCCTGCTCGTCTGATAATCCAACAGACGATCAAAAGC | |
| | TCCTTAACTGTCCGCAGGTTCTTGGTCTCTGAGCTTCGTGCATCACGAGG | |
| | TGCGGTGAAAGAAGGATCCCCTTACTATGCGGCTGTTGGGGATATCCAC | |
| | GCTTACATCTTTAACGCAGGACTGACACCATTCTTGACTACCTTAAGAT | |
| | ACGGGATAGGCACCAAATATGCTGCTGTTGCACTCAGTGTGTTCGCTGC | |
| | AGACATTGCAAAATTAAAGAGTCTACTTACCCTATACCAGGACAAGGGT | |
| | GTGGAGGCCGGATACATGGCACTCCTCGAAGATCCAGACTCTATGCACT | |
| | TTGCGCCTGGAAACTTCCCACACATGTACTCCTACGCGATGGGGGTGGC | |
| | TTCTTACCATGACCCCAGCATGCGCCAGTACCAATATGCTAGGAGGTTC | |
| | CTCAGCCGTCCTTTCTACTTGCTAGGGAGGGACATGGCTGCCAAGAACA | |
| | CAGGCACGCTGGATGAGCAACTGGCAAAGGAACTACAAGTGTCAGAAA | |
| | GAGACCGTGCCGCATTGTCCGCTGCGATTCAATCAGCAATGGAGGGGG | |
| | GAGAATCTGACGACTTCCCACTATCGGGATCCATGCCGGCTCTCTCCGA | |
| | CAATGCGCAACCAGTTACCCCAAGAACTCAACAGTCCCAGCTCTCCCCT | |
| | CCCCAATCATCAAGCATGTCTCAATCAGCGCCCAGGACCCCGGACTACC | |
| | AGCCTGATTTTGAACTGTAGGCTGCATCCACGCACCAACAGCAGGCCAA | |
| | AGAAACCACCCCCCTCCTCACACATCCCACCCAATCACCCGCCAAGACC | |
| | CAATCCAACACCCCAGCATCCCCCTCATTTAATTAAAAACTGACCAATA | |
| | GGGTGGGGAAGGAGAGTTATTGGCTATTGCCAAGTTCGTGCAGCAATG | |
| | GATTTTACCGATATTGATGCTGTCAACTCATTAATTGAATCATCATCAGC | |
| | AATCATAGATTCCATACAGCATGGAGGGCTGCAACCATCAGGCACTGTC | |
| | GGCCTATCACAAATCCCAAAGGGGATAACCAGCGCCTTAACCAAGGCC | |
| | TGGGAGGCCGAGGCAGCAACTGCTGGCAACGGGGACACCCAACACAAA | |
| | TCTGACAGTCCGGAAGACCATCAGGCCAACGACGCAGACTCCCCCGAA | |
| | GACACAGGCACCAACCAGACCATCCAAGAAGCCAATATCGTTGAAACA | |
| | CCCCACCCCGAAGTGCTATCGGCAGCCAAAGCCAGACTCAAGAGGCCC | |
| | AAGACAGGGAGGGACACCCACGACAATCCCTCTGCGCAACCTGATCAT | |
| | CTTTTAAAGGGGGGCCCCCTGAGCCCACAACCAGCGGCACCGTGGGTG | |
| | AAAGATCCATCCATTCATGGAGGTCCCGGCACCGCCGATCCCCGCCCAT | |
| | CACAAACTCAGGATCATTCCCTCACCGGAGAGAGATGGCAATCGTCACC | |
| | GACAAAGCAACCGGAGACATCGAACTGGTGGAATGGTGCAACCCGGGG | |
| | TGCACAGCTATCCGAGCTGAACCAACCAGACTCGACTGTGTATGCGGAC | |
| | ACTGCCCCACCATCTGCAGCCTCTGCATGTATGACGACTGATCAGGTAC | |
| | AACTATTAATGAAGGAGGTTGCCGACATGAAATCACTCCTTCAGGCACT | |
| | AGTGAGGAACCTAGCTGTCCTGCCTCAACTAAGGAATGAGGTTGCAGCA | |
| | ATCAGGACATCACAGGCCATGATAGAGGGGACACTCAATTCAATCAAG | |
| | ATTCTCGACCCTGGGAATTATCAAGAATCATCACTAAACAGTTGGTTCA | |
| | AACCACGCCAAGATCACGCGGTTGTTGTGTCCGGACCAGGGAATCCATT | |
| | GGCCATGCCAACCCCGATCCAAGACAACACCATATTCCTAGATGAACTG | |
| | GCAAGACCTCATCCTAGTTTGGTCAATCCGTCCCCGCCCGCTACCAACA | |
| | CCAATGCTGATCTTGGCCCACAGAAGCAGGCTGCGATAGCTTATATCTC | |
| | AGCAAAATGCAAGGATCAAGGGAAACGAGACCAGCTCTCAAAGCTCAT | |
| | CGAGCGAGCAACCACCCTGAGCGAGATCAACAAAGTCAAAAGACAGGC | |
| | CCTTGGCCTCTAGACCACTCGACCACCCCCAGTGATGAATACAACAATA | |
| | ATCAGAACCTCCCTAAACCACATGGCCAACCCAGCGCACCATCCACACC | |
| | ACCTATTACTACCCTTCGCCAGAAACTCCGCCGCAGCCGATTTATTCAA | |
| | AAGAAGCCACTCGATATGACTTAGCAACCGCAAGATAGGGTGGGGAAG | |
| | GTGCTTTACCTGCAAGAGGGCTCCCTCATCTTCAGACACGCACCCGCCA | |
| | ACCCACCAGTGACGCAATGGCAGACATGGACACTGTATATATCAATCTG | |
| | ATGGCAGATGATCCAACCCACCAAAAAGAACTGCTGTCCTTTCCCCTCA | |
| | TTCCCGTGACTGGTCCTGACGGGAAAAAGGAACTCCAACACCAGGTCCG | |
| | GACTCAATCCTTGCTCGCCTCAGACAAGCAAACTGAGAGGTTCATCTTC | |
| | CTCAACACTTACGGGTTTATCTATGACACTACACCGGACAAGACAACTT | |
| | TTTCTACCCCAGAGCATATCAATCAACCCAAGAGAACGATGGTGAGTGC | |
| | TGCAATGATGACCATCGGCCTGGTCCCCGCCAATATACCCTTGAACGAA | |
| | CTAACAGCTACTGTGTTTGGCCTGAAAATAAGAGTGAGGAAGAGTGCG | |
| | AGATATCGAGAGGTGGTCTGGTACCAGTGCAACCCTGTACCAGCCCTGC | |
| | TTGCAGCCACAAGGTTTGGTCGCCAAGGAGGTCTCGAATCGAGCACTGG | |
| | AGTTAGTGTAAGGGCCCCCGAGAAGATAGACTGCGAGAAGGATTATAC | |
| | TTACTACCCTTATTTCCTATCTGTGTGCTACATCGCCACTTCCAACCTGTT | |
| | CAAGGTACCAAAAATGGTCGCTAATGCGACCAACAGTCAATTATACCAC | |
| | CTGACCATGCAGATCACATTTGCCTTTCCAAAAAACATCCCCCCAGCTA | |
| | ACCAGAAACTCCTGACACTAGTGGATGAAGGATTCGAGGGCACTGTGG | |
| | ACTGCCATTTTGGGAACATGCTGAAAAAGGATCGGAAAGGGAACATGA | |
| | GGACACTGTCGCAGGCGGCAGACAAGGTCAGACGGATGAACATCCTTG | |
| | TTGGTATCTTTGACTTGCATGGGCCAACACTCTTCCTGGAGTACACCGG | |
| | GAAGCTAACAAAAGCTCTGTTAGGGTTCATGTCTACCAGCCGAACAGCA | |
| | ATCATCCCCATATCTCAGCTCAATCCTATGCTGAGTCAACTCATGTGGA | |
| | GCAGTGATGCCCAGATAGTAAAATTAAGAGTGGTCATAACTACATCCAA | |
| | ACGCGGCCCATGCGGGGGTGAGCAGGAGTATGTGCTGGATCCCAAATT | |
| | CACTGTTAAAAAAGAGAAAGCCCGACTCAACCCTTTCAAGAAGGCAGC | |
| | CCAATGATCAAATCTACAAGATCTCAGGAATCAGACCACTCTATACTAT | |
| | CCACTGATCAATAGACATGTAGCTATACAGTTGATGAACCTATGAAGAA | |
| | TCAGTTAGAAAACCGAATCCTTACTAGGGTGGGGAAGGAGTTGATTGG | |
| | GTGTCTAAACAAAAACATTCCTTTACACCTCCTCGCCACGAAACAACCA | |
| | TAATGAGGTTATCACGCACAATCCTGACCTTGATTCTCGGCACACTTACT | |
| | GATTATTTAATGGGTGCTCACTCCACCAATGTAACTGAGAGACCAAAGT | |
| | CTGAGGGGATTAGGGGTGATCTTACACCAGGCGCAGGTATCTTTGTAAC | |
| | TCAAGTCCGACAACTACAGATCTACCAACAGTCTGGGTATCATGACCTT | |
| | GTCATCAGATTATTACCTCTTCTACCGGCAGAACTCAATGATTGTCAAA | |
| | GGGAAGTTGTCACAGAGTACAACAATACGGTATCACAGCTGTTGCAGCC | |
| | TATCAAAACCAACCTGGATACCTTACTGGCTGGTGGTGGCACAAGGGAT | |
| | GCCGATATACAGCCGCGGTTCATTGGGGCAATCATAGCCACAGGTGCCC | |
| | TGGCGGTGGCTACGGTAGCTGAGGTGACTGCAGCCCAAGCACTATCTCA | |
| | GTCGAAAACAAACGCTCAAAATATTCTCAAGTTGAGGGATAGTATTCAG | |
| | GCCACCAACCAGGCAGTTTTCGAAATTTCACAAGGACTCGAGGCAACTG | |
| | CAACTGTGCTATCAAAACTGCAAACTGAGCTCAATGAGAACATTATCCC | |
| | AAGCCTGAACAACTTGTCCTGTGCTGCCATGGGTAATCGCCTTGGTGTA | |
| | TCACTATCACTCTACTTGACCTTAATGACCACCCTATTTGGGGACCAGAT | |
| | CACAAACCCAGTGCTGACACCGATCTCCTATAGCACTCTATCGGCAATG | |
| | GCAGGTGGTCATATTGGCCCGGTAATGAGTAAAATATTAGCCGGATCTA | |
| | TCACAAGTCAGTTGGGGGCGGAACAGTTGATTGCTAGCGGCTTAATACA | |
| | GTCACAGGTAGTAGGTTATGATTCCCAATACCAATTATTGGTTATCAGG | |
| | GTCAACCTTGTACGGATTCAAGAGGTCCAGAATACGAGAGTCGTATCAC | |
| | TAAGAACACTAGCAGTCAATAGGGACGGTGGACTCTATAGAGCCCAGG | |
| | TGCCTCCCGAGGTAGTTGAACGGTCTGGCATTGCAGAACGATTTTATGC | |
| | AGATGATTGTGTTCTTACTACAACCGATTACATTTGCTCATCGATCCGAT | |
| | CTTCTCGGCTTAATCCAGAGTTAGTTAGATGTCTCAGTGGGGCACTTGAT | |
| | TCATGCACATTTGAGAGGGAAAGTGCATTATTGTCAACCCCTTTCTTTGT | |
| | ATACAACAAGGCAGTTGTCGCAAATTGTAAAGCAGCAACATGTAGATG | |
| | TAATAAACCGCCGTCTATTATTGCCCAATACTCTGCATCAGCTCTGGTCA | |
| | CCATCACCACCGACACCTGTGCCGACCTCGAAATTGAGGGTTATCGCTT | |
| | CAACATACAGACTGAATCCAACTCATGGGTTGCACCAAACTTCACTGTC | |
| | TCGACTTCACAGATTGTATCAGTTGATCCCATAGACATCTCTTCTGACAT | |
| | TGCCAAAATCAACAGTTCCATCGAGGCTGCAAGAGAGCAGCTGGAACT | |
| | AAGCAACCAGATCCTTTCCCGGATCAACCCACGAATCGTGAATGATGAA | |
| | TCACTGATAGCTATTATCGTGACAATTGTTGTGCTTAGTCCCCTCGTAAT | |
| | CGGTCTGATTGTTGTTCTCGGTGTGATGTATAAGAATCTTAGGAAAGTC | |
| | CAACGAGCTCAAGCTGCCATGATGATGCAGCAAATGAGCTCATCACAG | |
| | CCTGTGACCACTAAATTAGGGACGCCTTTCTAGGAGAACAACCATATCA | |
| | CTCCACTCAATGATGAGCAAGACGTACCAATCATCAATGATTGTGTCAC | |
| | AAGGCCGGTTGGGAATGCATCGAATCTCTCCCCTTTCTTTTTAATTAAAA | |
| | ACATTTGAAGTGAAGATGAGAGGGGGGAAGTGTATGGTAGGGTGGGGA | |
| | AGGCAGCCAATTCCTGCCCATTAGGCCGACCGTATCAAAAGGATTCAAT | |
| | AGAAGTCTAGGTACAGGGTAACATGGAGGGCAGCCGCGATAATCTTAC | |
| | AGTGGATGATGAATTAAAGACAACATGGAGGTTAGCTTATAGAGTTGTG | |
| | TCTCTTCTATTGATGGTGAGCGCTTTGATAATCTCTATAGTAATCCTGAC | |
| | GAGAGATAACAGCCAAAGCATAATCACGGCGATCAACCAGTCATCTGA | |
| | CGCAGACTCTAAGTGGCAAACGGGAATAGAAGGGAAAATCACCTCCAT | |
| | TATGGCTGATACGCTCGATACCAGGAATGCAGTTCTTCTCCACATTCCA | |
| | CTCCAGCTCAACACTCTTGAGGCGAACCTATTGTCTGCCCTTGGGGGCA | |
| | ACACAGGAATTGGCCCCGGAGATCTAGAGCACTGCCGTTACCCTGTTCA | |
| | TGACACCGCTTACCTGCATGGAGTTAATCGATTACTCATCAATCAGACA | |
| | GCTGATTATACAGCAGAAGGCCCCCTAGATCATGTGAACTTCATTCCAG | |
| | CCCCGGTTACGACTACTGGATGCACAAGGATACCATCCTTTTCCGTGTC | |
| | ATCGTCCATTTGGTGCTATACACATAACGTGATTGAAACCGGTTGCAAT | |
| | GACCACTCAGGTAGTAATCAATATATCAGCATGGGAGTCATTAAGAGA | |
| | GCGGGCAACGGCCTACCTTACTTCTCAACAGTTGTAAGTAAGTATCTGA | |
| | CTGATGGGTTGAATAGGAAAAGCTGTTCTGTGGCTGCCGGATCTGGGCA | |
| | TTGCTACCTCCTTTGCAGCTTAGTGTCGGAGCCCGAACCTGATGACTATG | |
| | TGTCACCTGATCCTACACCGATGAGGTTAGGGGTGCTAACGTGGGATGG | |
| | ATCTTACACTGAACAGGTGGTACCCGAAAGAATATTCAGGAACATATGG | |
| | AGTGCAAACTACCCAGGAGTAGGGTCAGGTGCTATAGTAGGAAATAAG | |
| | GTGTTATTCCCATTTTACGGCGGAGTGAGGAATGGATCGACCCCGGAGG | |
| | TGATGAATAGGGGAAGGTACTACTACATCCAGGATCCAAATGACTATTG | |
| | CCCTGACCCGCTGCAAGATCAGATCTTAAGGGCGGAACAATCGTATTAC | |
| | CCAACTCGATTCGGTAGGAGGATGATAATGCAGGGGGTCCTAGCATGTC | |
| | CAGTATCCAACAATTCAACAATAGCAAGCCAATGTCAATCTTACTATTT | |
| | TAATAACTCATTAGGGTTCATTGGAGCAGAATCTAGAATCTATTACCTC | |
| | AATAGTAACATTTACCTTTATCAGAGGAGCTCGAGCTGGTGGCCTCACC | |
| | CCCAGATTTACCTGCTTGATTCTAGGATTGCAAGTCCGGGTACTCAGAA | |
| | CATTGACTCAGGTGTCAATCTCAAGATGTTAAACGTCACTGTGATTACA | |
| | CGACCATCATCTGGTTTTTGTAATAGTCAGTCACGATGCCCTAATGACTG | |
| | CTTATTCGGGGTCTACTCGGATATCTGGCCTCTTAGCCTTACCTCGGATA | |
| | GCATATTCGCGTTCACTATGTATTTACAGGGGAAGACAACACGTATTGA | |
| | CCCGGCTTGGGCGCTATTCTCCAATCATGCGATTGGGCATGAGGCTCGT | |
| | CTGTTTAATAAGGAGGTTAGTGCTGCTTATTCTACCACCACTTGTTTTTT | |
| | GGACACCATCCAAAACCAGGTGTATTGCCTGAGTATACTTGAGGTCAGG | |
| | AGTGAGCTCTTGGGAGCATTCAAAATAGTACCATTCCTCTATCGTGTCTT | |
| | GTAGGCATCCATTCGGCCAAAAAACTTGAGTGACTATGAGGTTAACACT | |
| | TGATCCCCCTTAAAGACACCTATCTAAATTACTGTCCTAGACCCATGATT | |
| | AGGTACCTTTTAAATCAATCATTTGGTTTTTAATTAAAAATGAAAAAAT | |
| | GGGCCTAGTTTCAAGAGAGGGCTGGAACCCACTAGGGTGGGGAAGGAT | |
| | TGCTTTGCTCCTTGACTCACACCCACGTATACTCGATCTCACTTCTGTAA | |
| | AGAAGGGATCCTTCTCAAACTCGCCCCACAATGTCCAATCAGGCAGCTG | |
| | AGATTATACTACCCACCTTCCATCTAGAATCACCCTTAATCGAGAATAA | |
| | GTGCTTTTATTATATGCAATTACTTGGTCTCGTGTTGCCACATGATCATT | |
| | GGAGATGGAGGGCATTCGTTAACTTTACAGTGGATCAGGTGCACCTTAA | |
| | AAATCGTAATCCCCGCTTAATGGCCCATATCGACCACACTAAAGATAGA | |
| | TTAAGGACTCATGGTGTCTTAGGTTTCCACCAGACTCAGACAAGTTTGA | |
| | GCCGTTATCGTGTTTTGCTCCATCCTGAAACCTTACCTTGGCTATCAGCC | |
| | ATGGGAGGATGCATCAATCAGGTTCCTAAAGCATGGCGGAATACTCTGA | |
| | AATCGATCGAGCATAGTGTAAAGCAGGAGGCACCTCAACTAAAGCTAC | |
| | TCATGGAGAGAACCTCATTAAAATTAACTGGAGTACCTTACTTGTTCTCT | |
| | AATTGCAATCCCGGGAAAACCACAGCAGGAACTATGCCTGTCCTAAGTG | |
| | AGATGGCATCGGAACTCTTGTCAAATCCTATCTCCCAATTCCAATCAAC | |
| | ATGGGGGTGTGCTGCTTCGGGGTGGCACCATGTAGTCAGTATCATGAGG | |
| | CTCCAACAATATCAAAGAAGGACAGGTAAGGAAGAGAAAGCAATCACC | |
| | GAAGTTCAGTATGGCACAGACACTTGTCTCATTAACGCAGACTATACCG | |
| | TTGTTTTTTCCACACAGAACCGTGTTATAACGGTCTTGCCCTTCGATGTT | |
| | GTCCTCATGATGCAAGACCTACTCGAATCCCGACGGAATGTTCTGTTCT | |
| | GTGCCCGCTTTATGTATCCCAGAAGCCAACTTCATGAGAGGATAAGTGC | |
| | AATATTAGCCCTTGGAGACCAACTGGGGAGAAAAGCACCCCAAGTCCT | |
| | GTATGATTTCGTGGCGACCCTCGAGTCATTTGCATACGCAGCTGTTCAA | |
| | CTTCATGACAACAATCCTACCTACGGTGGGGCCTTCTTTGAATTCAATAT | |
| | CCAAGAGTTAGAATCTATTCTGTCCCCTGCACTTAGTAAGGATCAGGTC | |
| | AACTTCTACATAGGTCAAGTTGTCTCAGCGTACAGTAACCTTCCTCCATC | |
| | TGAATCGGCAGAATTGTTGTGCCTGCTACGCCTGTGGGGTCATCCCTTG | |
| | CTAAACAGCCTTGATGCAGCAAAGAAAGTCAGGGAGTCTATGTGTGCC | |
| | GGGAAGGTTCTCGATTACAACGCCATTCGACTCGTCTTGTCTTTTTACCA | |
| | TACATTGTTAATCAATGGGTACCGAAAGAAACACAAGGGTCGCTGGCC | |
| | AAATGTGAATCAACATTCACTCCTCAACCCGATAGTGAGGCAGCTCTAT | |
| | TTTGATCAGGAAGAGATCCCACACTCTGTTGCCCTTGAGCACTATTTGG | |
| | ATGTCTCAATGATAGAATTTGAAAAAACTTTTGAAGTGGAACTATCTGA | |
| | CAGCCTAAGCATCTTCCTGAAGGATAAGTCGATAGCTTTGGATAAGCAA | |
| | GAATGGTACAGTGGTTTTGTCTCAGAAGTGACTCCGAAGCACCTACGAA | |
| | TGTCTCGTCATGATCGCAAGTCTACCAATAGGCTCCTGTTAGCTTTCATT | |
| | AACTCCCCTGAATTCGACGTTAAGGAGGAGCTTAAGTACTTGACTACGG | |
| | GTGAGTACGCCACTGACCCAAATTTCAATGTCTCATACTCACTTAAAGA | |
| | GAAGGAAGTAAAAAAAGAAGGGCGCATATTCGCAAAAATGTCACAAAA | |
| | GATGAGAGCATGCCAGGTTATTTGTGAAGAATTGCTAGCACATCATGTG | |
| | GCTCCTTTGTTTAAAGAGAATGGTGTTACTCAATCAGAGCTATCCCTGA | |
| | CAAAAAATTTGTTGGCTATTAGCCAACTGAGTTACAACTCGATGGCTGC | |
| | TAAGGTGCGATTGCTGAGGCCAGGGGACAAGTTCACTGCTGCACACTAT | |
| | ATGACCACAGACCTAAAGAAGTACTGTCTCAATTGGCGGCACCAGTCAG | |
| | TCAAACTGTTCGCCAGAAGCCTGGATCGACTGTTTGGATTAGACCATGC | |
| | GTTTTCTTGGATACATGTCCGTCTCACCAACAGCACTATGTACGTTGCTG | |
| | ACCCCTTCAATCCACCAGACTCAGAGGCATGCACAGATTTAGACGACAA | |
| | TAAGAACACCGGGATTTTTATTATAAGTGCAAGAGGTGGTATAGAAGGC | |
| | CTCCAACAAAAATTATGGACTGGCATATCGATTGCAATTGCCCAAGCGG | |
| | CAGCGGCCCTCGAAGGCTTACGAATTGCTGCTACTCTGCAGGGGGATAA | |
| | CCAAGTTTTGGCGATTACGAAGGAATTCATGACCCCAGTCCCAGAGGAT | |
| | GTAATCCATGAGCAGCTATCTGAGGCGATGTCTCGATACAAAAGGACTT | |
| | TCACATACCTCAATTATTTAATGGGGCATCAGTTGAAGGATAAAGAAAC | |
| | CATCCAATCCAGTGACTTCTTTGTTTATTCCAAAAGAATCTTCTTCAATG | |
| | GATCGATCTTAAGTCAATGCCTCAAAAACTTCAGTAAACTCACTACTAA | |
| | TGCCACTACCCTTGCTGAGAATACTGTGGCCGGCTGCAGTGACATCTCT | |
| | TCATGCATTGCCCGTTGTGTGGAAAACGGGTTGCCTAAGGATGCCGCAT | |
| | ATATCCAGAATATAATCATGACTCGGCTTCAACTATTGCTAGATCATTA | |
| | CTATTCAATGCATGGCGGCATAAATTCAGAATTAGAGCAGCCAACTTTA | |
| | AGTATCTCTGTTCGAAACGCAACCTACTTACCATCTCAACTAGGCGGTT | |
| | ACAATCATTTGAATATGACCCGACTATTCTGCCGCAATATCGGCGACCC | |
| | GCTTACCAGTTCTTGGGCGGAGTCAAAAAGACTAATGGATGTTGGTCTC | |
| | CTCAGTCGTAAGTTCTTAGAGGGGATATTATGGAGACCCCCGGGAAGTG | |
| | GGACGTTTTCAACACTCATGCTTGACCCGTTCGCACTTAACATTGATTAC | |
| | CTGAGGCCGCCAGAGACAATTATCCGAAAACACACCCAAAAAGTCTTG | |
| | TTGCAAGATTGCCCAAATCCCCTATTAGCAGGTGTCGTTGACCCGAACT | |
| | ACAACCAAGAATTAGAGCTGTTAGCTCAGTTCTTGCTTGATCGGGAAAC | |
| | CGTTATTCCCAGGGCTGCCCATGCCATCTTCGAGTTATCTGTCTTGGGAA | |
| | GGAAAAAACATATACAAGGATTGGTAGATACTACAAAGACAATTATTC | |
| | AGTGCTCATTGGAAAGACAGCCATTGTCTTGGAGGAAAGTTGAGAACAT | |
| | TGTTACCTACAACGCGCAGTATTTCCTCGGGGCCACCCAACAGGCTGAT | |
| | ACTAATGTCTCAGAAGGGCAGTGGGTGATGCCAGGTAACCTTAAGAAG | |
| | CTTGTGTCCCTCGACGATTGCTCGGTCACGCTGTCTACTGTATCACGGCG | |
| | CATATCATGGGCCAATCTACTGAACTGGAGAGCTATAGATGGTCTGGAA | |
| | ACCCCGGATGTGATAGAGAGTATTGATGGTCGCCTTGTACAATCATCCA | |
| | ATCAATGTGGCCTATGTAATCAAGGGTTGGGATCCTACTCCTGGTTTTTC | |
| | TTGCCCTCTGGGTGTGTGTTCGACCGTCCACAAGATTCTCGGGTAGTTCC | |
| | AAAGATGCCATACGTGGGGTCCAAAACAGATGAGAGACAGACTGCATC | |
| | AGTGCAAGCTATACAAGGATCCACTTGTCACCTCAGGGCAGCATTGAGG | |
| | CTTGTATCACTCTACCTATGGGCCTATGGAGATTCTGACATATCATGGCT | |
| | AGAAGCTGCAACGCTGGCTCAAACACGGTGCAATGTCTCTCTCGATGAT | |
| | TTGCGAATCTTGAGCCCTCTTCCTTCTTCGGCGAATTTACACCACAGATT | |
| | AAATGACGGGGTAACACAGGTTAAATTCATGCCCGCCACATCTAGCCGA | |
| | GTGTCAAAGTTCGTCCAAATTTGCAATGACAACCAGAATCTTATCCGTG | |
| | ATGATGGGAGTGTTGATTCCAATATGATTTATCAACAGGTTATGATATT | |
| | AGGGCTTGGAGAGATTGAATGCTTGTTAGCTGACCCAATTGATACAAAC | |
| | CCAGAACAATTGATTCTTCATCTACACTCTGATAATTCTTGCTGTCTCCG | |
| | GGAGATGCCAACGACCGGCTTTGTACCTGCTCTAGGACTAACCCCATGT | |
| | TTAACTGTCCCAAAGCATAATCCTTACATTTATGACGATAGCCCAATAC | |
| | CCGGTGATTTGGATCAGAGGCTCATTCAGACCAAATTTTTCATGGGGTC | |
| | TGACAATTTGGATAATCTTGATATCTACCAGCAGCGAGCTTTACTGAGT | |
| | AGGTGTGTAGCTTATGATGTCATCCAATCGATCTTTGCCTGTGATGCACC | |
| | AGTCTCTCAGAAGAATGACGCAATCCTTCACACTGATTACCATGAGAAT | |
| | TGGATCTCAGAGTTCCGATGGGGTGACCCTCGTATTATCCAAGTAACGG | |
| | CAGGCTATGAGTTAATTCTGTTCCTTGCATACCAGCTTTATTATCTCAGA | |
| | GTGAGGGGTGACCGTGCAATCCTGTGCTATATCGACAGGATACTCAATA | |
| | GGATGGTATCTTCCAATCTAGGTAGTCTCATCCAGACACTCTCTCATCCA | |
| | GAGATTAGGAGGAGATTCTCGTTGAGTGATCAAGGGTTTCTTGTTGAAA | |
| | GAGAACTAGAGCCAGGTAAGCCCTTGGTTAAACAAGCGGTTATGTTCTT | |
| | AAGGGACTCGGTCCGCTGCGCTTTAGCAACTATCAAGGCAGGAATTGAG | |
| | CCTGAAATCTCCCGAGGTGGTTGTACTCAGGATGAGCTGAGCTTTACTC | |
| | TTAAGCACTTACTATGTCGGCGTCTCTGTGTAATCGCTCTCATGCATTCA | |
| | GAAGCAAAGAACTTGGTTAAAGTTAGAAACCTTCCTGTAGAAGAGAAA | |
| | ACCGCCTTATTGTACCAGATGTTGGTCACTGAGGCCAATGCTAGGAAAT | |
| | CAGGGTCTGCCAGCATTATCATAAACCTAGTCTCGGCACCCCAGTGGGA | |
| | CATTCATACACCAGCATTGTATTTTGTGTCAAAGAAAATGCTAGGGATG | |
| | CTTAAGAGGTCAACCACACCCTTGGATATAAGTGACCTCTCTGAGAACC | |
| | AGAACCCCGCACCTGCAGAGCTTAGTGATGCTCCTGGTCACATGGCAGA | |
| | AGAATTCCCCTGTTTGTTTAGTAGTTATAACGCTACATATGAAGACACA | |
| | ATCACTTACAATCCAATGACTGAAAAACTCGCCTTGCATTTGGACAACA | |
| | GTTCCACCCCATCCAGAGCACTTGGTCGTCACTACATCCTGCGGCCTCTT | |
| | GGGCTTTACTCATCCGCATGGTACCGGTCTGCGGCACTACTAGCGTCAG | |
| | GGGCCCTAAATGGGTTGCCTGAGGGGTCGAGCCTGTATTTAGGAGAAG | |
| | GGTACGGGACCACCATGACTCTGCTTGAGCCCGTTGTCAAGTCTTCAAC | |
| | TGTTTACTACCATACATTGTTTGACCCAACCCGGAACCCTTCACAGCGG | |
| | AACTATAAACCAGAACCACGGGTATTCACGGATTCTATTTGGTACAAGG | |
| | ATGATTTCACACGGCCACCCGGTGGTATTATCAACCTGTGGGGTGAAGA | |
| | TATACGTCAGAGTGATATCACACAGAAAGACACGGTCAACTTCATACTA | |
| | TCTCAGATCCCGCCAAAATCACTTAAGTTGATACACGTTGATATTGAGT | |
| | TCTCACCAGACTCCGATGTACGGACACTACTATCCGGCTATTCTCATTGT | |
| | GCACTATTGGCCTACTGGCTATTGCAACCTGGAGGGCGATTCGCAGTTA | |
| | GGGTTTTCTTAAGTGACCATATCATAGTTAACTTGGTCACTGCGATCCTG | |
| | TCTGCTTTTGACTCCAATTTGGTGTGCATTGCGTCAGGATTGACACACAA | |
| | GGATGATGGGGCAGGTTATATTTGCGCGAAAAAGCTTGCAAATGTTGAG | |
| | GCTTCAAGAATTGAGTACTACTTGAGGATGGTCCATGGTTGTGTTGACT | |
| | CATTAAAGATCCCTCATCAATTAGGAATCATTAAATGGGCCGAGGGTGA | |
| | GGTGTCCCAGCTTACCAGAAAGGCGGATGATGAAATAAATTGGCGGTT | |
| | AGGTGATCCAGTTACCAGATCATTTGATCCAGTTTCTGAGCTAATAATT | |
| | GCACGAACAGGGGGGTCTGTATTAATGGAATACGGGGCTTTTACTAACC | |
| | TCAGGTGTGCGAACTTGGTAGATACATACAAACTTCTGGCTTCAATTGT | |
| | AGAGACCACCCTAATGGAAATAAGGGTTGAGCAAGATCAGTTGGAAGA | |
| | TAGTTCGAGGAGACAAATCCAAGTAATCCCCGCTTTCAACACAAGATCT | |
| | GGGGGAAGGATCCGTACACTGATTGAGTGTGCTCAGCTGCAGATTATAG | |
| | ATGTTATTTGTGTAAACATAGATCACCTCTTTCCTAAACACCGACATGTT | |
| | CTTGTCACACAACTTACCTACCAGTCGGTGTGCCTTGGGGATTTGATTGA | |
| | AGGTCCCCAAATTAAGACGTATCTAAGGGCCAGAAAGTGGATCCAACG | |
| | TCGGGGACTCAATGAGACAGTTAACCATATCATCACTGGACAAGTGTCA | |
| | CGGAATAAAGCAAGGGATTTTTTTAAGAGGCGCCTGAAGTTGGTTGGCT | |
| | TTTCACTCTGCGGAGGTTGGAGCTACCTCTCACTTTAGCTGTTCAGGTTG | |
| | CTGATCATCATGAACAATCGGAGTCGGAATCGTAAACAGAAAGTCACA | |
| | AAATTGTGGATAAACAATGATTGCATTAGTATTTAATAAAAAATATGTC | |
| | TTTTATTTCGT | |
| Avian | ACGAAAAAGAAGAATAAAAGGCAGAAGCCTTTTAAAAGGAACCCTGGG | 82 |
| paramyxovirus | CTGTCGTAGGTGTGGGAAGGTTGTATTCCGAGTGCGCCTCCGAGGCATC | |
| 4 strain | TACTCTACACCTATCACAATGGCTGGTGTCTTTTCCCAGTATGAGAGGTT | |
| APMV- | TGTGGACAATCAATCTCAGGTGTCAAGGAAGGATCATCGGTCCTTAGCA | |
| 4/duck/Dela- | GGAGGGTGCCTTAAAGTGAACATCCCTATGCTTGTCACTGCATCCGAAG | |
| ware/549227 | ACCCCACCACGCGTTGGCAACTAGCATGCTTATCTCTGAGGCTCTTGATT | |
| /2010, | TCCAATTCATCAACCAGTGCTATCCGCCAGGGAGCAATACTGACCCTCA | |
| complete | TGTCATTGCCATCGCAAAACATGAGAGCAACAGCAGCTATTGCTGGGTC | |
| genome | CACGAATGCGGCTGTTATCAACACTATGGAAGTCTTAAGTGTCAATGAC | |
| Genbank: | TGGACCCCATCTTTTGACCCAAGAAGTGGTCTATCTGAGGAGGACGCTC | |
| JX987283.1 | AGGTGTTCAGAGACATGGCAAGAGATCTGCCTCCTCAGTTCACTTCTGG | |
| | ATCACCCTTTACATCAGCATTGGCGGAGGGGTTTACTCCCGAGGACACT | |
| | CATGACCTGATGGAGGCACTGACTAGTGTACTGATACAGATCTGGATTC | |
| | TGGTGGCCAAGGCCATGACCAATATTGATGGATCTGGGGAGGCTAACG | |
| | AAAGACGCCTTGCAAAATACATCCAAAAGGGACAGCTCAATCGTCAGT | |
| | TTGCAATTGGCAATCCTGCCCGTCTGATAATCCAACAGACAATCAAAAG | |
| | CTCATTAACTGTCCGCAGGTTCTTGGTCTCTGAGCTCCGCGCATCACGTG | |
| | GTGCAGTAAAGGAGGGTTCCCCTTACTATGCAGCCGTTGGGGATATCCA | |
| | CGCTTACATCTTCAATGCAGGATTGACACCATTCTTGACCACCCTGAGA | |
| | TATGGCATTGGCACCAAGTACGCCGCTGTCGCACTCAGTGTGTTTGCTG | |
| | CAGACATTGCAAAATTGAAGAGTCTACTCACCCTGTATCAAGACAAAGG | |
| | TGTAGAAGCTGGATACATGGCACTCCTTGAAGATCCAGATTCCATGCAC | |
| | TTTGCACCTGGAAACTTCCCACACATGTATTCCTATGCGATGGGAGTGG | |
| | CCTCCTATCACGACCCTAGCATGCGCCAATACCAGTATGCCAGGAGGTT | |
| | TCTCAGTCGTCCCTTCTACCTGCTAGGAAGAGACATGGCTGCTAAGAAC | |
| | ACAGGAACTCTGGATGAGCAGCTGGCGAAAGAACTGCAAGTGTCAGAG | |
| | AGGGACCGCGCTGCACTGTCTGCCGCGATTCAATCAGCAATGGAGGGG | |
| | GGAGAGTCAGATGACTTCCCATTGTCAGGATCCATGCCGGCCCTCTCTG | |
| | AGAGCACACAACCGGTCACCCCCAGGACTCAACAGTCCCAGCTCTCTCC | |
| | TCCTCAATCATCAAACATGTCCCAATCGGCGCCTAGGACCCCGGACTAT | |
| | CAACCCGACTTTGAGCTGTAGACTATATCCACACACCGACAATAGCTCC | |
| | AGAAGACCCCCTTCCCCCCCATACACCCCACCCGGTCATCCACAAAGAC | |
| | CCAGTCCAACATCCCAGCACTATTCCCTTTTAATTAAAAACTGGCCGAC | |
| | AGGGTGGGGAAGGAGGACTGTTAGCTGCCACCAACGGTGTGCAGCAAT | |
| | GGATTTTACAGACATTGACGCTGTCAACTCACTGATTGAGTCATCATCG | |
| | GCAATTATAGACTCCATACAGCATGGAGGGCTGCAACCAGCAGGCACT | |
| | GTTGGCTTATCTCAAATTCCAAAAGGGATAACCAGTGCACTGAATAAAG | |
| | CCTGGGAAGCTGAGGCGGCAACTGCCGGCAGTGGAGACACCCAACACA | |
| | AACCCGATGACCCAGAGGACCACCAGGCTAGGGACACGGAGTCCCTGG | |
| | AAGACACAGGCAACGACCCGGCCACACAGGGGACTAACATTGTTGAGA | |
| | CACCCCACCCAGAAGTACTGTCAGCAGCCAAAGCTAGACTCAAGAGAC | |
| | CCAAAGCAGGGAAAGACACCCATGGCAATCCCCCCACTCAACCCGATC | |
| | ACTTTTTAAAGGGGGGCCTCCCGAGTCCACAACCGACAGCACCGCGGAT | |
| | GCAAAGTCCACCCAACCATGGAAGCTCCAGCACCGCCGATCCCCGCCA | |
| | ATCACAAACTCAGGATCATTCCCCCACCGGAGAGAAATGGCAATTGTCA | |
| | CCGACAAAGCAACCGGAGACATCGAACTGGTGGAGTGGTGCAACCCAG | |
| | GGTGTACAGCAGTCCGAATTGAACCAGCCAGACTTGACTGTGTATGCGG | |
| | ACACTGCCCCACCATCTGCAGTCTCTGCATGTATGACGACTGATCAGGT | |
| | ACAGTTGTTGATGAAGGAGGTTGCTGACATAAAATCACTCCTCCAGGCA | |
| | CTAGTAAGGAATCTAGCTGTCTTGCCCCAACTAAGGAATGAGGTTGCAG | |
| | CAATCAGAACATCACAGGCCATGATAGAGGGGACACTCAATTCAATTA | |
| | AGATTCTTGATCCTGGAAATTATCAGGAATCATCACTAAACAGTTGGTT | |
| | CAAACCTCGCCAGGAACACACTGTTATTGTGTCAGGACCAGGGAATCCA | |
| | CTGGCCATGCCGACTCCAGTTCAGGACAGTACCATATTCTTAGATGAGC | |
| | TAGCAAGACCTCATCCTAATTTGGTCAATCCGTCTCCGCCCGTCACCAG | |
| | CACCAATGTTGACCTTGGCCCACAGAAGCAGGCTGCAATAGCCTACGTT | |
| | TCCGCCAAGTGCAAGGACCCAGGGAAACGGGACCAGCTTTCAAGGCTT | |
| | ATTGAACGGGCGGCTACCTTGAGTGAGATCAACAAGGTTAAAAGACAG | |
| | GCTCTCGGGCTCTAAATTAATCAACCACCCGTTGCAACGATCGAGACAA | |
| | CAATAAAAATCCCCCTGAATCACATGACCAAATCTGCATACCACTCACA | |
| | TCATCCGCCTATACCCCTCACCATAAATACCACCTTAGCCGATTTATTTA | |
| | AAAGAAATCATTCATCACAACCTGGTAATCATAAACTAGGGTGGGGAA | |
| | GGTCTCTTGTCTGCAGGAAGGCTCCTCTGTCTCCAGGCACGCACCCGTC | |
| | AACCCACCAATAACACAATGGCGGACATGGACACGATATACATCAACT | |
| | TGATGGCAGATGATCCAACCCATCAAAAAGAATTGCTGTCATTCCCTCT | |
| | GATTCCAGTGACTGGACCTGATGGGAAGAAAGTGCTCCAACACCAGAT | |
| | CCGGACCCAATCCTTGCTCACCTCAGACAAACAAACGGAGAGGTTCATC | |
| | TTTCTCAACACTTACGGGTTCATCTATGACACAACCCCGGACAAGACAA | |
| | CTTTTTCCACCCCTGAGCATATCAATCAGCCTAAGAGGACAATGGTGAG | |
| | TGCTGCGATGATGACTATTGGTCTGGTTCCTGCTACAATACCCCTGAATG | |
| | AATTGACGGCCACTGTGTTTAACCTTAAAGTAAGAGTGAGGAAAAGTGC | |
| | GAGGTATCGAGAAGTGGTTTGGTACCAGTGCAACCCCGTACCAGCTCTG | |
| | CTCGCAGCCACCAGATTTGGCCGCCAAGGGGGTCTTGAGTCGAGCACCG | |
| | GAGTCAGTGTAAAGGCACCTGAGAAGATTGATTGTGAGAAAGATTATA | |
| | CTTACTACCCTTATTTCCTATCTGTGTGCTACATCGCCACTTCCAACCTCT | |
| | TTAAGGTACCGAAGATGGTTGCCAATGCAACCAACAGTCAATTGTATCA | |
| | CCTAACCATGCAGGTCACATTTGCATTTCCGAAAAACATTCCCCCAGCC | |
| | AATCAGAAACTCCTGACACAGGTAGATGAAGGATTTGAGGGTACCGTG | |
| | GATTGCCATTTTGGGAACATGCTAAAAAAGGATAGGAAAGGGAACATG | |
| | AGGACTTTGTCTCAAGCAGCAGATAAGGTCAGAAGAATGAATATCCTTG | |
| | TGGGAATATTTGACTTGCACGGACCTACACTATTCCTGGAATATACTGG | |
| | GAAATTGACAAAAGCCCTGTTGGGGTTCATGTCCACCAGCCGAACAGCA | |
| | ATCATCCCCATATCACAACTCAATCCTATGCTGAGTCAACTCATGTGGA | |
| | GCAGTGACGCCCAGATAGTAAAGTTACGGGTGGTCATCACTACATCTAA | |
| | ACGTGGCCCGTGTGGGGGCGAGCAGGAATATGTGCTGGATCCTAAATTC | |
| | ACAGTTAAGAAAGAAAAGGCTCGACTCAATCCATTCAAGAAGGCAGCC | |
| | TAATAATTAAACCTACAAGATCCCAAGAATTAAACAGCTCTATACAATT | |
| | CATAGGTTGATAGAAATGCCACTACACAGCTAATGATTTTCCAGAAAAT | |
| | CACTTAGAAAACCAAATCCTTATTAGGGTGGGGAAGTAGTTGATTGGGT | |
| | GTCTAAACAAAAGTGCTTCTTTGCAACTCCCCACCCCGAAGCAATCACA | |
| | ATGAGACCATTAAACACGCTTTTGACCGTGATTCTTATCATACTCATCAG | |
| | CTATTTGGTGATTGTTCATTCTAGTGATGCGGTTGAGAGGCCAAGGACT | |
| | GAGGGAATTAGGGGCGACCTCATTCCAGGTGCGGGTATCTTCGTGACTC | |
| | AAGTCCGACAACTGCAAATCTATCAGCAGTCAGGGTACCACGACCTTGT | |
| | CATAAGATTATTACCCCTTTTACCAACGGAACTCAATGATTGCCAAAAA | |
| | GAAGTAGTCACAGAATACAATAATACAGTATCACAATTGTTGCAGCCTA | |
| | TCAAAACCAACTTGGATACCCTATTAGCAGATGGTAATACGAGGGAAG | |
| | CGGATATACAGCCGCGGTTTATTGGAGCAATAATAGCCACAGGTGCCTT | |
| | GGCGGTAGCAACAGTGGCAGAAGTAACTGCAGCTCAGGCACTCTCCCA | |
| | GTCCAAAACAAATGCTCAAAATATTCTCAAGCTAAGAGATAGTATCCAG | |
| | GCCACCAACCAAGCGGTCTTTGAAATTTCACAAGGGCTTGAGGCAACTG | |
| | CAACTGTGCTATCGAAACTACAGACAGAGCTCAATGAGAATATTATCCC | |
| | AAGCCTGAACAATTTATCCTGTGCTGCCATGGGGAATCGTCTTGGTGTA | |
| | TCACTCTCACTCTATTTAACTCTAATGACTACCCTCTTTGGGGACCAAAT | |
| | TACGAACCCAGTGCTGACACCAATTTCTTACAGCACACTATCGGCAATG | |
| | GCAGGTGGTCATATTGGCCCAGTGATGAGTAAAATATTAGCCGGATCGG | |
| | TCACGAGCCAGTTGGGGGCAGAACAATTGATTGCTAGTGGCTTAATACA | |
| | ATCACAGGTGGTAGGCTATGATTCCCAGTATCAATTATTGGTAATCAGG | |
| | GTTAACCTTGTTCGGATTCAGGAAGTCCAGAATACCAGGGTTGTATCAT | |
| | TAAGAACGCTAGCTGTCAATAGAGATGGTGGACTTTATAGAGCCCAAGT | |
| | TCCACCTGAGGTAGTCGAACGATCCGGCATTGCAGAGCGGTTTTACGCA | |
| | GATGATTGTGTTCTCACCACGACCGACTATATTTGCTCATCAATCAGATC | |
| | CTCTCGGCTTAATCCAGAATTAGTCAAGTGTCTCAGTGGGGCACTTGAT | |
| | TCATGTACATTCGAGAGGGAGAGTGCCCTGTTATCAACTCCTTTCTTTGT | |
| | GTACAATAAGGCTGTCGTAGCAAATTGCAAAGCGGCAACATGCAGATG | |
| | CAACAAACCACCGTCAATTATTGCTCAATATTCTGCATCAGCTCTAGTA | |
| | ACCATCACCACTGACACCTGTGCCGATCTCGAAATTGAGGGTTACCGTT | |
| | TCAACATACAGACTGAATCTAACTCGTGGGTTGCACCTAACTTTACTGT | |
| | CTCAACCTCACAGATAGTGTCAGTTGATCCAATAGACATATCCTCTGAC | |
| | ATCGCAAAAATCAACAATTCGATTGAGGCCGCACGAGAGCAGCTAGAA | |
| | CTGAGCAACCAGATCCTATCCCGGATTAACCCCCGAATCGTGAATGACG | |
| | AATCACTGATAGCTATTATCGTGACAATTGTTGTGCTTAGTCTCCTTGTA | |
| | GTCGGTCTTATCATTGTTCTCGGCGTGATGTATAAAAATCTCAAGAAGG | |
| | TCCAACGAGCTCAGGCTGCTATGATGATGCAGCAAATGAGTTCATCGCA | |
| | GCCTGTAACCACAAAACTGGGGACACCCTTCTAGGTGAATAAATGCATC | |
| | ACCTCTTTCCTTGATGAGCGAGATGTCTTAATCATTGATAATTATGCCGT | |
| | AAGGCTGGTAGGGAATGTGCTGAATCTCTCCTCTTCCTTTTTAATTAAAA | |
| | ACGGTTGAACTGAGGGGGAGAATGTGCATGGTAGGGTGGGGAAGGTGT | |
| | CTGATTCCTACCTATCGGGCCAACTGTACCAGTAGAAGCTAACAGGAAT | |
| | TCTAATGCAGAGTGACATGGAGGGCAGTCGTGATAACCTCACAGTGGAT | |
| | GATGAGTTAAAGACAACATGGAGGTTAGCTTACAGAGTTGTATCTCTCC | |
| | TATTAATGGTGAGTGCTTTGATAATTTCTATAGTAATCTTGACGAGGGAT | |
| | AACAGCCAAAGCATAATCACGGCAATCAACCAGTCATATGATGCAGAC | |
| | TCAAAGTGGCAAACAGGGATAGAGGGGAAAATCACCTCTATCATGACT | |
| | GATACGCTTGATACTAGGAATGCAGCTCTCCTCCACATTCCACTCCAAC | |
| | TTAATACACTTGAAGCAAACCTATTATCAGCCCTCGGTGGCAACACAGG | |
| | AATCGGCCCCGGGGATCTAGAGCATTGCCGTTATCCAGTTCATGATTCT | |
| | GCTTACCTGCATGGAGTCAACCGATTACTTATCAATCAAACGGCTGATT | |
| | ATACAGCAGAGGGTCCACTAGATCATGTGAACTTCATACCGGCACCAGT | |
| | TACGACCACTGGATGCACTAGGATACCATCTTTTTCCGTGTCCTCATCCA | |
| | TTTGGTGTTATACTCACAATGTGATTGAAACTGGTTTTAATGATCACTCA | |
| | GGCAGCAATCAGTATATTAGCATGGGGGTGATTAAGAGGGCTGGCAAC | |
| | GGCTTGCCTTATTTCTCAACCGTTGTGAGTAAGTATCTGACCGACGGATT | |
| | GAATAGGAAAAGTTGTTCTGTGGCTGCTGGGTCTGGGCATTGCTATCTT | |
| | CTCTGCAGCCTAGTATCAGAGCCCGAGCCTGACGACTATGTATCACCAG | |
| | ACCCCACACCGATGAGGTTAGGGGTTCTGACATGGGATGGGTCCTATAC | |
| | TGAACAGGTGGTGCCTGAAAGGATATTCAAAAACATATGGAGTGCAAA | |
| | TTACCCTGGGGTGGGATCAGGTGCTATTGTGGGAAATAAGGTGTTGTTC | |
| | CCATTTTACGGAGGAGTGAGGAATGGGTCGACACCTGAGGTTATGAATA | |
| | GGGGAAGGTATTACTACATTCAAGATCCTAATGATTATTGTCCTGATCC | |
| | ACTGCAAGACCAAATCTTAAGGGCAGAACAATCATATTATCCTACACGG | |
| | TTTGGTAGGAGGATGGTGATGCAGGGTGTCTTAGCGTGCCCAGTGTCCA | |
| | ACAACTCAACAATTGCCAGCCAATGCCAGTCCTACTATTTCAACAACTC | |
| | ATTAGGGTTCATTGGGGCGGAATCTAGGATTTATTACCTAAATGGGAAC | |
| | CTCTACCTTTACCAAAGAAGCTCGAGCTGGTGGCCCCACCCCCAGATTT | |
| | ATCTGCTTGACCCCAGAATTGCAAGCCCGGGCACTCAGAACATCGACTC | |
| | AGGCATTAATCTCAAGATGTTGAATGTTACCGTTATTACACGACCGTCA | |
| | TCTGGTTTTTGTAATAGTCAGTCAAGATGCCCTAATGACTGCTTATTCGG | |
| | GGTCTATTCAGACGTCTGGCCTCTTAGCCTAACCTCAGATAGTATATTCG | |
| | CATTCACGATGTATTTACAAGGGAAGACAACACGTATTGACCCGGCGTG | |
| | GGCACTGTTCTCCAATCACGCAATTGGGCATGAAGCTCGTCTATTCAAC | |
| | AAGGAGGTCAGTGCTGCTTACTCCACTACCACTTGCTTTTCGGACACCA | |
| | TCCAAAACCAGGTGTATTGCCTGAGTATACTTGAAGTTAGAAGTGAGCT | |
| | TTTGGGGCCATTCAAGATAGTACCATTCCTCTACCGTGTCCTATAGGTGC | |
| | CTGCTCGATCGAGAACTCCAAATAATCGTGGAATTAGTACTTAATCTTC | |
| | CCTATGGATATCTGCCTTAATTACTGTCCTAGGTCTCTGGATTAGCGCCC | |
| | TTTAAACCAGTTTTTTGATTTTTAATTAAAAATAGAAGATTAGACCTGGA | |
| | CTCGGGGAGGGAGAAGAACCTATTAGGGTGGGGAAGGATTACTTTACT | |
| | CCATGACTCACAATCGCACACACCTGACCTCATTTCCACTGAGAAGGAA | |
| | CCCTCCTCAAATTTGATTTGCAATGTCCAATCAAGCAGCTGAGATTATA | |
| | CTCCCTACCTTTCACCTAGAGTCACCCTTAATCGAGAACAAATGCTTCTA | |
| | CTATATGCAATTACTTGGTCTTATGTTGCCGCATGATCATTGGAGATGGA | |
| | GGGCATTTGTCAACTTTACAGTGGATCAAGCACACCTTAGAAACCGTAA | |
| | TCCTCGCTTGATGGCCCACATCGACCACACTAAGGATAAACTAAGGGCT | |
| | CATGGTGTCTTAGGTTTCCATCAGACCCAAACAGGTGAGAGCCGTTTCC | |
| | GTGTCTTGCTTCACCCGGAAACCTTACCATGGCTATCAGCAATGGGAGG | |
| | ATGCATAAACCAAGTCCCCAAAGCATGGCGGAACACTCTGAAGTCCATC | |
| | GAGCACAGTGTGAAGCAGGAGGCAACACAACTACAATCGCTTATGAAA | |
| | AAAACCTCATTGAAATTAACAGGAGTACCCTACTTATTTTCCAACTGTA | |
| | ATCCCGGGAAAACCACAACAGGCACTATGCCTGTATTAAGCGAGATGG | |
| | CATCAGAGCTCCTATCAAATCCCATCTCCCAATTCCAATCAACATGGGG | |
| | GTGTGCTGCTTCAGGGTGGCACCATATTGTTAGCATCATGAGGCTTCAA | |
| | CAGTATCAAAGAAGGACAGGTAAAGAGGAGAAGGCGATCACTGAGGTT | |
| | CATTTTGGTTCAGACACCTGTCTCATTAATGCAGACTACACCGTTATCTT | |
| | TTCCTTACAGAGCCGTGTAATAACAGTTTTACCTTTTGACGTTGTCCTCA | |
| | TGATGCAAGACCTGCTCGAATCTCGACGAAATGTCCTGTTCTGTGCCCG | |
| | CTTTATGTACCCCAGAAGCCAATTGCATGAGAGGATAAGCATGATACTA | |
| | GCTCTCGGAGATCAACTTGGGAAAAAGGCACCCCAAGTTCTATATGACT | |
| | TTGTTGCAACCCTTGAATCATTTGCATACGCAGCTGTCCAACTTCATGAC | |
| | AATAACCCTATCTACGGTGGGACTTTCTTTGAATTCAATATCCAAGAATT | |
| | AGAATCTATCTTGTCTCCTGCGCTTAGCAAGGACCAGGTCAACTTCTAC | |
| | ATTAGTCAGGTTGTCTCAGCATACAGTAACCTCCCCCCATCTGAATCGG | |
| | CAGAATTGCTATGCCTGTTACGCCTATGGGGTCACCCTTTACTAAATAG | |
| | CCTCGATGCAGCAAAGAAAGTCAGAGAATCAATGTGTGCCGGGAAGGT | |
| | TCTTGACTACAATGCCATTCGATTAGTCTTGTCTTTTTACCATACATTATT | |
| | GATCAATGGATATCGGAAGAAACACAAGGGACGCTGGCCAAATGTGAA | |
| | TCAACATTCACTACTCAACCCAATAGTGAGGCAGCTTTACTTTGATCAA | |
| | GAAGAGATCCCACATTCTGTCGCCCTCGAACATTACTTAGACATCTCAA | |
| | TGATAGAATTTGAGAAAACTTTTGAGGTTGAACTATCTGACAGCCTAAG | |
| | CATCTTTTTGAAAGACAAGTCGATTGCCTTGGACAAACAAGAGTGGTAC | |
| | AGCGGTTTTGTTTCAGAAGTGACCCCAAAGCACTTGCGGATGTCTCGTC | |
| | ATGACCGCAAGTCCACCAACAGGCTCCTGCTGGCCTTTATCAACTCCCC | |
| | TGAATTCGATGTTAAAGAAGAGCTAAAATACTTGACTACAGGTGAGTAT | |
| | GCTACTGATCCAAATTTCAACGTTTCTTACTCACTTAAAGAGAAGGAAG | |
| | TAAAGAAAGAAGGACGAATCTTTGCAAAAATGTCACAAAAGATGAGAG | |
| | CGTGCCAGGTTATTTGTGAAGAGTTGCTAGCACATCATGTAGCCCCTTT | |
| | GTTTAAAGAGAATGGTGTCACACAGTCGGAACTATCTCTGACAAAAAAT | |
| | CTGCTAGCTATCAGTCAGTTGAGTTATAACTCAATGGCTGCTAAGGTGC | |
| | GGTTGCTGAGACCAGGGGACAAATTCACTGCCGCACACTATATGACCAC | |
| | AGACCTGAAAAAGTACTGCCTTAATTGGCGTCACCAGTCAGTCAAACTG | |
| | TTTGCCAGAAGCCTAGATCGACTGTTCGGGCTAGATCATGCTTTTTCTTG | |
| | GATACATGTCCGCCTCACCAACAGCACCATGTATGTGGCTGATCCATTC | |
| | AATCCACCAGACTCAGATGCATGCCCAAACTTAGACGACAACAAAAAC | |
| | ACGGGAATTTTCATCATAAGTGCACGAGGTGGGATAGAAGGCCTCCAA | |
| | CAAAAACTGTGGACCGGCATATCAATCGCAATCGCGCAAGCAGCTGCA | |
| | GCCCTCGAAGGCTTGAGAATTGCTGCTACTTTGCAGGGGGACAACCAGG | |
| | TTCTAGCGATCACGAAGGAATTTGTAACCCCAGTCCCGGAAGGTGTCCT | |
| | CCATGAGCAATTATCTGAGGCGATGTCCCGATATAAAAAGACTTTCACA | |
| | TACCTTAATTACTTAATGGGGCATCAACTGAAAGATAAAGAGACAATCC | |
| | AATCCAGTGATTTCTTTGTTTACTCTAAAAGGATATTCTTTAATGGGTCC | |
| | ATTCTGAGTCAATGTCTCAAAAACTTCAGTAAGCTCACCACTAATGCCA | |
| | CCACCCTTGCCGAGAACACTGTAGCCGGCTGCAGTGACATCTCATCATG | |
| | CATCGCTCGTTGTGTAGAAAACGGGTTGCCAAAGGATGCTGCATACATC | |
| | CAGAACATAGTCATGACTCGACTTCAACTGTTGCTAGATCACTACTATT | |
| | CCATGCATGGTGGCATAAACTCAGAATTAGAACAGCCGACCCTAAGTAT | |
| | TTCTGTTCGGAATGCAACCTATTTACCATCTCAGTTGGGCGGTTACAATC | |
| | ATCTAAATATGACCCGACTATTTTGCCGCAACATCGGTGACCCGCTCAC | |
| | TAGTTCCTGGGCAGAAGCAAAGAGACTAATGGAAGTTGGCCTGCTCAAT | |
| | CGTAAATTCCTGGAGGGAATATTGTGGCGACCTCCGGGAAGTGGGACAT | |
| | TCTCAACACTTATGCTTGACCCGTTTGCGCTGAACATTGATTACCTCAGA | |
| | CCACCAGAGACAATAATCCGAAAGCATACCCAGAAGGTCTTGCTGCAA | |
| | GATTGCCCTAATCCCCTATTAGCCGGTGTGGTTGATCCGAACTACAACC | |
| | AGGAACTGGAACTATTAGCGCAGTTCTTGCTCGACCGAGAGACCGTTAT | |
| | TCCCAGGGCAGCTCATGCTATCTTTGAGCTGTCTGTCTTGGGGAGGAAA | |
| | AAACATATACAAGGGTTGGTGGACACTACAAAAACGATTATCCAGTGTT | |
| | CGCTGGAAAGACAACCATTGTCCTGGAGGAAAGTTGAGAACATTATCA | |
| | CCTATAATGCGCAGTATTTCCTTGGAGCCACTCAGCAGATTGATACAGA | |
| | TTCCCCTGAAAAGCAGTGGGTGATGCCAAGCAACTTCAAGAAGCTCGTG | |
| | TCTCTTGACGATTGTTCAGTCACATTGTCTACTGTTTCCCGGCGTATATC | |
| | TTGGGCCAACCTACTTAATTGGAGGGCAATAGATGGCTTGGAAACCCCA | |
| | GATGTGATAGAAAGTATTGATGGGCGCCTTGTGCAATCATCCAATCAGT | |
| | GTGGCCTATGTAATCAAGGATTAAGTTCCTACTCCTGGTTCTTCCTCCCC | |
| | TCCGGATGTGTGTTTGATCGTCCACAAGACTCCAGGGTAGTACCGAAAA | |
| | TGCCGTATGTGGGATCCAAGACAGATGAGAGGCAGACTGCGTCGGTAC | |
| | AAGCTATACAGGGATCCACATGTCACCTTAGAGCAGCATTGAGACTTGT | |
| | ATCACTCTACCTTTGGGCTTATGGGGATTCTGATATATCATGGCTGGAA | |
| | GCCGCGACACTAGCCCAAACACGGTGCAATATTTCCCTTGATGATCTGC | |
| | GAATCCTGAGCCCTCTACCTTCCTCGGCAAATTTACACCACAGATTAAA | |
| | TGACGGGGTAACACAAGTGAAATTCATGCCTGCTACATCAAGCCGAGTA | |
| | TCAAAGTTTGTCCAGATTTGCAATGACAACCAGAATCTTATCCGTGATG | |
| | ATGGGAGTGTGGATTCCAATATGATTTATCAGCAAGTCATGATATTAGG | |
| | ACTTGGGGAATTTGAGTGCTTGTTGGCCGACCCAATCGATACTAACCCA | |
| | GAGCAATTGATTCTTCATCTACACTCTGACAATTCTTGCTGCCTCCGGGA | |
| | GATGCCAACAACCGGCTTTGTGCCTGCTTTGGGATTAACCCCATGCTTA | |
| | ACTGTACCAAAGCAAAATCCATATATTTATGACGAGAGTCCAATACCTG | |
| | GTGACCTGGATCAACGGCTCATCCAAACAAAGTTTTTCATGGGTTCTGA | |
| | TAATCTAGACAACCTTGATATCTATCAGCAACGAGCGTTACTAAGTCGG | |
| | TGTGTGGCTTATGATGTTATCCAATCAGTATTTGCTTGTGATGCACCAGT | |
| | TTCTCAGAAGAATGATGCAATCCTCCATACTGACTATCATGAGAATTGG | |
| | ATCTCAGAGTTCCGATGGGGTGACCCTCGGATAATTCAAGTGACAGCAG | |
| | GTTATGAATTGATCTTGTTTCTTGCTTACCAGCTTTATTACCTTAGAGTG | |
| | AGGGGTGACCGTGCAATCCTGTGCTATATTGATAGGATACTGAATAGGA | |
| | TGGTGTCATCAAATCTAGGCAGCCTTATCCAGACACTCTCCCATCCGGA | |
| | GATTAGGAGGAGGTTTTCATTAAGTGATCAAGGATTCCTTGTTGAAAGG | |
| | GAACTAGAGCCAGGCAAACCTTTGGTAAAACAAGCAGTCATGTTCCTAA | |
| | GGGACTCAGTCCGATGTGCTTTAGCAACTATCAAGGCAGGAGTCGAGCC | |
| | GGAGATCTCCCGAGGTGGCTGTACCCAAGATGAGTTGAGTTTCACCCTC | |
| | AAGCACTTGCTATGTCGACGTCTCTGTATAATTGCTCTCATGCATTCAGA | |
| | AGCAAAGAACTTGGTCAAGGTCAGAAATCTCCCAGTAGAGGAAAAATC | |
| | TGCTTTACTATACCAGATGTTGGTCACCGAAGCTAATGCCCGGAAATCA | |
| | GGATCTGCTAGCATCATCATAGGCTTAATTTCGGCACCTCAGTGGGATA | |
| | TCCATACCCCAGCACTGTACTTTGTATCAAAGAAGATGCTAGGAATGCT | |
| | CAAAAGGTCAACTACACCATTGGATGTAAATGATCTGTCTGAGAGCCAG | |
| | GACCTTATGCCAACAGAGTTGAGTGATGGTCCTGGTCACATGGCAGAGG | |
| | GATTTCCCTGTCTATTTAGTAGTTTTAACGCTACATATGAAGACACAATT | |
| | GTTTATAATCCGATGACTGAAAAGCCTGCAGTACATTTGGACAATGGAT | |
| | CCACCCCATCCAGGGCGCTAGGTCGCCACTACATCTTGCGGCCCCTCGG | |
| | GCTTTACTCGTCTGCATGGTACCGGTCTGCAGCACTCTTAGCATCAGGTG | |
| | CTCTCAATGGGTTACCGGAGGGATCAAGCCTATACTTGGGAGAAGGGTA | |
| | TGGGACCACCATGACTCTGCTCGAACCCGTCGTCAAGTCCTCAACTGTT | |
| | TATTACCACACATTGTTTGACCCGACCCGGAATCCCTCACAGCGGAATT | |
| | ACAAACCAGAGCCGCGAGTCTTCACTGATTCCATCTGGTACAAGGATGA | |
| | CTTCACACGACCGCCTGGTGGCATTGTAAATCTATGGGGTGAAGATGTG | |
| | CGTCAGAGTGACGTCACACAGAAAGACACAGTTAATTTCATATTATCCC | |
| | GGATCCCACCCAAATCACTCAAACTGATCCATGTTGACATTGAATTCTC | |
| | ACCAGACTCCAATGTACGGACACTACTATCTGGTTACTCCCATTGCGCA | |
| | TTATTGGCCTACTGGCTATTGCAACCTGGAGGGCGATTTGCGGTTAGGG | |
| | TCTTCCTGAGTGACCATCTCTTAGTAAACTTGGTCACTGCTATTCTGTCT | |
| | GCTTTCGACTCTAATCTACTGTGTATTGCATCTGGATTGACACACAAAG | |
| | ATGATGGGGCAGGTTACATTTGTGCTAAGAAGCTTGCCAATGTTGAGGC | |
| | ATCAAGGATTGAGCACTACTTAAGGATGGTCCATGGTTGCGTTGATTCA | |
| | TTAAAGATCCCCCACCAACTAGGGATCATTAAGTGGGCTGAAGGTGAG | |
| | GTGTCTCGGCTCACAAAAAAGGCAGATGAAGAAATAAATTGGCGATTA | |
| | GGTGACCCGGTTACTAGATCATTTGATCCAGTTTCCGAGTTAATAATCG | |
| | CACGGACAGGGGGGTCTGTATTAATGGAATATGGGACTTTCATTAATCT | |
| | CAGGTGTTCAAACCTGGCAGATACATATAAACTTTTGGCTTCAATCGTG | |
| | GAGACCACCTTGATGGAGATAAGGGTTGAACAAGATCAATTGGAAGAC | |
| | AACTCAAGAAGACAAATTCAGGTGGTCCCCGCCTTTAATACGAGATCCG | |
| | GGGGGAGGATCCGTACATTGATTGAGTGTGCCCAGCTGCAGGTTATAGA | |
| | TGTCATATGTGTAAACATAGATCACCTCTTCCCCAAACATCGACATGTTC | |
| | TTGTTACACAACTCACTTACCAGTCAGTGTGCCTTGGAGACTTGATCGA | |
| | GGGGCCCCAAATTAAGATGTATCTAAGGGCCAGGAAGTGGATCCAACG | |
| | TAGAGGACTCAATGAGACAATTAACCATATCATCACTGGACAGATATCA | |
| | CGAAATAAGGCAAGGGATTTCTTCAAGAGGCGCCTGAAGTTGGTTGGCT | |
| | TCTCGCTTTGCGGCGGTTGGAGTTACCTCTCACTTTAGTTACTTAGGTTG | |
| | TTGATCATTGTGAAAAATCGGAGTCGGAATCGCAAATAAAAACATACA | |
| | AAATTGCAAATTTACAATAATCGCATTAATATTTAATAAAAAATATGTC | |
| | TTTTATTTCGT | |
| Newcastle di- | ACCAAACAGAGAATCCGTGAGTTACGATAAAAGGCGAAAGAGCAATTG | 83 |
| sease virus | AAGTCACACGGGTAGAAGGTGTGAATCTCGAGTGCGAGCCCGAAGCAC | |
| strain | AAACTCGAGAAAGCCTTCTGCCAACATGTCCTCCGTATTTGATGAGTAC | |
| LaSota, | GAACAGCTCCTCGCGGCTCAGACTCGCCCCAACGGAGCTCATGGAGGG | |
| complete | GGAGAAAAAGGGAGTACCTTAAAAGTAGACGTCCCGGTATTCACTCTTA | |
| genome with | ACAGTGATGACCCAGAAGATAGATGGAGCTTTGTGGTATTCTGCCTCCG | |
| modification | GATTGCTGTTAGCGAAGATGCCAACAAACCACTCAGGCAAGGTGCTCTC | |
| in 5408- | ATATCTCTTTTATGCTCCCACTCACAGGTAATGAGGAACCATGTTGCCCT | |
| 5409-5410 | TGCAGGGAAACAGAATGAAGCCACATTGGCCGTGCTTGAGATTGATGG | |
| nucleotides | CTTTGCCAACGGCACGCCCCAGTTCAATAATAGGAGTGGAGTGTCTGAA | |
| resulting in | GAGAGAGCACAGAGATTTGCGATGATAGCAGGATCTCTCCCTCGGGCAT | |
| L289A | GCAGCAACGGAACCCCGTTCGTCACAGCCGGGGCCGAAGATGATGCAC | |
| substitution | CAGAAGACATCACCGATACCCTGGAGAGGATCCTCTCTATCCAGGCTCA | |
| | AGTATGGGTCACAGTAGCAAAAGCCATTACTGCGTATGAGACTGCAGAT | |
| | GAGTCGGAAACAAGGCGAATCAATAAGTATATGCAGCAAGGCAGGGTC | |
| | CAAAAGAAATACATCCTCTACCCCGTATGCAGGAGCACAATCCAACTCA | |
| | CGATCAGACAGTCTCTTGCAGTCCGCATCTTTTTGGTTAGCGAGCTCAA | |
| | GAGAGGCCGCAACACGGCAGGTGGTACCTCTACTTATTATAACCTGGTA | |
| | GGGGACGTAGACTCATACATCAGGAATACCGGGCTTACTGCATTCTTCT | |
| | TGACACTCAAGTACGGAATCAACACCAAGACATCAGCCCTTGCACTTAG | |
| | TAGCCTCTCAGGCGACATCCAGAAGATGAAGCAGCTCATGCGTTTGTAT | |
| | CGGATGAAAGGAGATAATGCGCCGTACATGACATTACTTGGTGATAGTG | |
| | ACCAGATGAGCTTTGCGCCTGCCGAGTATGCACAACTTTACTCCTTTGCC | |
| | ATGGGTATGGCATCAGTCCTAGATAAAGGTACTGGGAAATACCAATTTG | |
| | CCAGGGACTTTATGAGCACATCATTCTGGAGACTTGGAGTAGAGTACGC | |
| | TCAGGCTCAGGGAAGTAGCATTAACGAGGATATGGCTGCCGAGCTAAA | |
| | GCTAACCCCAGCAGCAAGGAGGGGCCTGGCAGCTGCTGCCCAACGGGT | |
| | CTCCGAGGAGACCAGCAGCATAGACATGCCTACTCAACAAGTCGGAGT | |
| | CCTCACTGGGCTTAGCGAGGGGGGGTCCCAAGCTCTACAAGGCGGATC | |
| | GAATAGATCGCAAGGGCAACCAGAAGCCGGGGATGGGGAGACCCAATT | |
| | CCTGGATCGGATGAGAGCGGTAGCAAATAGCATGAGGGAGGCGCCAAA | |
| | CTCTGCACAGGGCACTCCCCAATCGGGGCCTCCCCCAACTCCTGGGCCA | |
| | TCCCAAGATAACGACACCGACTGGGGGTATTGATGGACAAAACCCAGC | |
| | CTGCTTCCACAAAAACATCCCAATGCCCTCACCCGTAGTCGACCCCTCG | |
| | ATTTGCGGCTCTATATGACCACACCCTCAAACAAACATCCCCCTCTTTCC | |
| | TCCCTCCCCCTGCTGTACAACTCCGCACGCCCTAGATACCACAGGCACA | |
| | ATGCGGCTCACTAACAATCAAAACAGAGCCGAGGGAATTAGAAAAAAG | |
| | TACGGGTAGAAGAGGGATATTCAGAGATCAGGGCAAGTCTCCCGAGTC | |
| | TCTGCTCTCTCCTCTACCTGATAGACCAGGACAAACATGGCCACCTTTAC | |
| | AGATGCAGAGATCGACGAGCTATTTGAGACAAGTGGAACTGTCATTGA | |
| | CAACATAATTACAGCCCAGGGTAAACCAGCAGAGACTGTTGGAAGGAG | |
| | TGCAATCCCACAAGGCAAGACCAAGGTGCTGAGCGCAGCATGGGAGAA | |
| | GCATGGGAGCATCCAGCCACCGGCCAGTCAAGACAACCCCGATCGACA | |
| | GGACAGATCTGACAAACAACCATCCACACCCGAGCAAACGACCCCGCA | |
| | TGACAGCCCGCCGGCCACATCCGCCGACCAGCCCCCCACCCAGGCCACA | |
| | GACGAAGCCGTCGACACACAGCTCAGGACCGGAGCAAGCAACTCTCTG | |
| | CTGTTGATGCTTGACAAGCTCAGCAATAAATCGTCCAATGCTAAAAAGG | |
| | GCCCATGGTCGAGCCCCCAAGAGGGGAATCACCAACGTCCGACTCAAC | |
| | AGCAGGGGAGTCAACCCAGTCGCGGAAACAGTCAGGAAAGACCGCAGA | |
| | ACCAAGTCAAGGCCGCCCCTGGAAACCAGGGCACAGACGTGAACACAG | |
| | CATATCATGGACAATGGGAGGAGTCACAACTATCAGCTGGTGCAACCCC | |
| | TCATGCTCTCCGATCAAGGCAGAGCCAAGACAATACCCTTGTATCTGCG | |
| | GATCATGTCCAGCCACCTGTAGACTTTGTGCAAGCGATGATGTCTATGA | |
| | TGGAGGCGATATCACAGAGAGTAAGTAAGGTCGACTATCAGCTAGATC | |
| | TTGTCTTGAAACAGACATCCTCCATCCCTATGATGCGGTCCGAAATCCA | |
| | ACAGCTGAAAACATCTGTTGCAGTCATGGAAGCCAACTTGGGAATGATG | |
| | AAGATTCTGGATCCCGGTTGTGCCAACATTTCATCTCTGAGTGATCTACG | |
| | GGCAGTTGCCCGATCTCACCCGGTTTTAGTTTCAGGCCCTGGAGACCCC | |
| | TCTCCCTATGTGACACAAGGAGGCGAAATGGCACTTAATAAACTTTCGC | |
| | AACCAGTGCCACATCCATCTGAATTGATTAAACCCGCCACTGCATGCGG | |
| | GCCTGATATAGGAGTGGAAAAGGACACTGTCCGTGCATTGATCATGTCA | |
| | CGCCCAATGCACCCGAGTTCTTCAGCCAAGCTCCTAAGCAAGTTAGATG | |
| | CAGCCGGGTCGATCGAGGAAATCAGGAAAATCAAGCGCCTTGCTCTAA | |
| | ATGGCTAATTACTACTGCCACACGTAGCGGGTCCCTGTCCACTCGGCAT | |
| | CACACGGAATCTGCACCGAGTTCCCCCCCGCAGACCCAAGGTCCAACTC | |
| | TCCAAGCGGCAATCCTCTCTCGCTTCCTCAGCCCCACTGAATGATCGCGT | |
| | AACCGTAATTAATCTAGCTACATTTAAGATTAAGAAAAAATACGGGTAG | |
| | AATTGGAGTGCCCCAATTGTGCCAAGATGGACTCATCTAGGACAATTGG | |
| | GCTGTACTTTGATTCTGCCCATTCTTCTAGCAACCTGTTAGCATTTCCGA | |
| | TCGTCCTACAAGACACAGGAGATGGGAAGAAGCAAATCGCCCCGCAAT | |
| | ATAGGATCCAGCGCCTTGACTTGTGGACTGATAGTAAGGAGGACTCAGT | |
| | ATTCATCACCACCTATGGATTCATCTTTCAAGTTGGGAATGAAGAAGCC | |
| | ACTGTCGGCATGATCGATGATAAACCCAAGCGCGAGTTACTTTCCGCTG | |
| | CGATGCTCTGCCTAGGAAGCGTCCCAAATACCGGAGACCTTATTGAGCT | |
| | GGCAAGGGCCTGTCTCACTATGATAGTCACATGCAAGAAGAGTGCAACT | |
| | AATACTGAGAGAATGGTTTTCTCAGTAGTGCAGGCACCCCAAGTGCTGC | |
| | AAAGCTGTAGGGTTGTGGCAAACAAATACTCATCAGTGAATGCAGTCA | |
| | AGCACGTGAAAGCGCCAGAGAAGATTCCCGGGAGTGGAACCCTAGAAT | |
| | ACAAGGTGAACTTTGTCTCCTTGACTGTGGTACCGAAGAAGGATGTCTA | |
| | CAAGATCCCTGCTGCAGTATTGAAGGTTTCTGGCTCGAGTCTGTACAAT | |
| | CTTGCGCTCAATGTCACTATTAATGTGGAGGTAGACCCGAGGAGTCCTT | |
| | TGGTTAAATCTCTGTCTAAGTCTGACAGCGGATACTATGCTAACCTCTTC | |
| | TTGCATATTGGACTTATGACCACCGTAGATAGGAAGGGGAAGAAAGTG | |
| | ACATTTGACAAGCTGGAAAAGAAAATAAGGAGCCTTGATCTATCTGTCG | |
| | GGCTCAGTGATGTGCTCGGGCCTTCCGTGTTGGTAAAAGCAAGAGGTGC | |
| | ACGGACTAAGCTTTTGGCACCTTTCTTCTCTAGCAGTGGGACAGCCTGCT | |
| | ATCCCATAGCAAATGCTTCTCCTCAGGTGGCCAAGATACTCTGGAGTCA | |
| | AACCGCGTGCCTGCGGAGCGTTAAAATCATTATCCAAGCAGGTACCCAA | |
| | CGCGCTGTCGCAGTGACCGCCGACCACGAGGTTACCTCTACTAAGCTGG | |
| | AGAAGGGGCACACCCTTGCCAAATACAATCCTTTTAAGAAATAAGCTGC | |
| | GTCTCTGAGATTGCGCTCCGCCCACTCACCCAGATCATCATGACACAAA | |
| | AAACTAATCTGTCTTGATTATTTACAGTTAGTTTACCTGTCTATCAAGTT | |
| | AGAAAAAACACGGGTAGAAGATTCTGGATCCCGGTTGGCGCCCTCCAG | |
| | GTGCAAGATGGGCTCCAGACCTTCTACCAAGAACCCAGCACCTATGATG | |
| | CTGACTATCCGGGTTGCGCTGGTACTGAGTTGCATCTGTCCGGCAAACT | |
| | CCATTGATGGCAGGCCTCTTGCAGCTGCAGGAATTGTGGTTACAGGAGA | |
| | CAAAGCCGTCAACATATACACCTCATCCCAGACAGGATCAATCATAGTT | |
| | AAGCTCCTCCCGAATCTGCCCAAGGATAAGGAGGCATGTGCGAAAGCC | |
| | CCCTTGGATGCATACAACAGGACATTGACCACTTTGCTCACCCCCCTTG | |
| | GTGACTCTATCCGTAGGATACAAGAGTCTGTGACTACATCTGGAGGGGG | |
| | GAGACAGGGGCGCCTTATAGGTGCCATTATTGGCGGTGTGGCTCTTGGG | |
| | GTTGCAACTGCCGCACAAATAACAGCGGCCGCAGCTCTGATACAAGCC | |
| | AAACAAAATGCTGCCAACATCCTCCGACTTAAAGAGAGCATTGCCGCA | |
| | ACCAATGAGGCTGTGCATGAGGTCACTGACGGATTATCGCAACTAGCAG | |
| | TGGCAGTTGGGAAGATGCAGCAGTTTGTTAATGACCAATTTAATAAAAC | |
| | AGCTCAGGAATTAGACTGCATCAAAATTGCACAGCAAGTTGGTGTAGA | |
| | GCTCAACCTGTACCTAACCGAATTGACTACAGTATTCGGACCACAAATC | |
| | ACTTCACCCGCTTTAAACAAGCTGACTATTCAGGCACTTTACAATCTAG | |
| | CTGGTGGAAATATGGATTACTTATTGACTAAGTTAGGTGTAGGGAACAA | |
| | TCAACTCAGCTCATTAATCGGTAGCGGCTTAATCACCGGTAACCCTATT | |
| | CTATACGACTCACAGACTCAACTCTTGGGTATACAGGTAACTGCCCCTT | |
| | CAGTCGGGAACCTAAATAATATGCGTGCCACCTACTTGGAAACCTTATC | |
| | CGTAAGCACAACCAGGGGATTTGCCTCGGCACTTGTCCCAAAAGTGGTG | |
| | ACACAGGTCGGTTCTGTGATAGAAGAACTTGACACCTCATACTGTATAG | |
| | AAACTGACTTAGATTTATATTGTACAAGAATAGTAACGTTCCCTATGTC | |
| | CCCTGGTATTTATTCCTGCTTGAGCGGCAATACGTCGGCCTGTATGTACT | |
| | CAAAGACCGAAGGCGCACTTACTACACCATACATGACTATCAAAGGTTC | |
| | AGTCATCGCCAACTGCAAGATGACAACATGTAGATGTGTAAACCCCCCG | |
| | GGTATCATATCGCAAAACTATGGAGAAGCCGTGTCTCTAATAGATAAAC | |
| | AATCATGCAATGTTTTATCCTTAGGCGGGATAACTTTAAGGCTCAGTGG | |
| | GGAATTCGATGTAACTTATCAGAAGAATATCTCAATACAAGATTCTCAA | |
| | GTAATAATAACAGGCAATCTTGATATCTCAACTGAGCTTGGGAATGTCA | |
| | ACAACTCGATCAGTAATGCTTTGAATAAGTTAGAGGAAAGCAACAGAA | |
| | AACTAGACAAAGTCAATGTCAAACTGACTAGCACATCTGCCCTCATTAC | |
| | CTATATCGTTTTGACTATCATATCTCTTGTTTTTGGTATACTTAGCCTGAT | |
| | TCTAGCATGCTACCTAATGTACAAGCAAAAGGCGCAACAAAAGACCTT | |
| | ATTATGGCTTGGGAATAATACTCTAGATCAGATGAGAGCCACTACAAAA | |
| | ATGTGAACACAGATGAGGAACGAAGGTTTCCCTAATAGTAATTTGTGTG | |
| | AAAGTTCTGGTAGTCTGTCAGTTCAGAGAGTTAAGAAAAAACTACCGGT | |
| | TGTAGATGACCAAAGGACGATATACGGGTAGAACGGTAAGAGAGGCCG | |
| | CCCCTCAATTGCGAGCCAGGCTTCACAACCTCCGTTCTACCGCTTCACCG | |
| | ACAACAGTCCTCAATCATGGACCGCGCCGTTAGCCAAGTTGCGTTAGAG | |
| | AATGATGAAAGAGAGGCAAAAAATACATGGCGCTTGATATTCCGGATT | |
| | GCAATCTTATTCTTAACAGTAGTGACCTTGGCTATATCTGTAGCCTCCCT | |
| | TTTATATAGCATGGGGGCTAGCACACCTAGCGATCTTGTAGGCATACCG | |
| | ACTAGGATTTCCAGGGCAGAAGAAAAGATTACATCTACACTTGGTTCCA | |
| | ATCAAGATGTAGTAGATAGGATATATAAGCAAGTGGCCCTTGAGTCTCC | |
| | GTTGGCATTGTTAAAAACTGAGACCACAATTATGAACGCAATAACATCT | |
| | CTCTCTTATCAGATTAATGGAGCTGCAAACAACAGTGGGTGGGGGGCAC | |
| | CTATCCATGACCCAGATTATATAGGGGGGATAGGCAAAGAACTCATTGT | |
| | AGATGATGCTAGTGATGTCACATCATTCTATCCCTCTGCATTTCAAGAAC | |
| | ATCTGAATTTTATCCCGGCGCCTACTACAGGATCAGGTTGCACTCGAAT | |
| | ACCCTCATTTGACATGAGTGCTACCCATTACTGCTACACCCATAATGTA | |
| | ATATTGTCTGGATGCAGAGATCACTCACATTCATATCAGTATTTAGCACT | |
| | TGGTGTGCTCCGGACATCTGCAACAGGGAGGGTATTCTTTTCTACTCTGC | |
| | GTTCCATCAACCTGGACGACACCCAAAATCGGAAGTCTTGCAGTGTGAG | |
| | TGCAACTCCCCTGGGTTGTGATATGCTGTGCTCGAAAGTCACGGAGACA | |
| | GAGGAAGAAGATTATAACTCAGCTGTCCCTACGCGGATGGTACATGGG | |
| | AGGTTAGGGTTCGACGGCCAGTACCACGAAAAGGACCTAGATGTCACA | |
| | ACATTATTCGGGGACTGGGTGGCCAACTACCCAGGAGTAGGGGGTGGA | |
| | TCTTTTATTGACAGCCGCGTATGGTTCTCAGTCTACGGAGGGTTAAAAC | |
| | CCAATTCACCCAGTGACACTGTACAGGAAGGGAAATATGTGATATACA | |
| | AGCGATACAATGACACATGCCCAGATGAGCAAGACTACCAGATTCGAA | |
| | TGGCCAAGTCTTCGTATAAGCCTGGACGGTTTGGTGGGAAACGCATACA | |
| | GCAGGCTATCTTATCTATCAAGGTGTCAACATCCTTAGGCGAAGACCCG | |
| | GTACTGACTGTACCGCCCAACACAGTCACACTCATGGGGGCCGAAGGC | |
| | AGAATTCTCACAGTAGGGACATCTCATTTCTTGTATCAACGAGGGTCAT | |
| | CATACTTCTCTCCCGCGTTATTATATCCTATGACAGTCAGCAACAAAAC | |
| | AGCCACTCTTCATAGTCCTTATACATTCAATGCCTTCACTCGGCCAGGTA | |
| | GTATCCCTTGCCAGGCTTCAGCAAGATGCCCCAACCCGTGTGTTACTGG | |
| | AGTCTATACAGATCCATATCCCCTAATCTTCTATAGAAACCACACCTTGC | |
| | GAGGGGTATTCGGGACAATGCTTGATGGTGTACAAGCAAGACTTAACCC | |
| | TGCGTCTGCAGTATTCGATAGCACATCCCGCAGTCGCATTACTCGAGTG | |
| | AGTTCAAGCAGTACCAAAGCAGCATACACAACATCAACTTGTTTTAAAG | |
| | TGGTCAAGACTAATAAGACCTATTGTCTCAGCATTGCTGAAATATCTAA | |
| | TACTCTCTTCGGAGAATTCAGAATCGTCCCGTTACTAGTTGAGATCCTCA | |
| | AAGATGACGGGGTTAGAGAAGCCAGGTCTGGCTAGTTGAGTCAATTAT | |
| | AAAGGAGTTGGAAAGATGGCATTGTATCACCTATCTTCCACGACATCAA | |
| | GAATCAAACCGAATGCCGGCGCGTGCTCGAATTCCATGTTGCCAGTTGA | |
| | CCACAATCAGCCAGTGCTCATGCGATCAGATTAAGCCTTGTCAATAGTC | |
| | TCTTGATTAAGAAAAAATGTAAGTGGCAATGAGATACAAGGCAAAACA | |
| | GCTCATGGTAAATAATACGGGTAGGACATGGCGAGCTCCGGTCCTGAA | |
| | AGGGCAGAGCATCAGATTATCCTACCAGAGTCACACCTGTCTTCACCAT | |
| | TGGTCAAGCACAAACTACTCTATTACTGGAAATTAACTGGGCTACCGCT | |
| | TCCTGATGAATGTGACTTCGACCACCTCATTCTCAGTCGACAATGGAAA | |
| | AAAATACTTGAATCGGCCTCTCCTGATACTGAGAGAATGATAAAACTCG | |
| | GAAGGGCAGTACACCAAACTCTTAACCACAATTCCAGAATAACCGGAG | |
| | TGCTCCACCCCAGGTGTTTAGAAGAACTGGCTAATATTGAGGTCCCAGA | |
| | TTCAACCAACAAATTTCGGAAGATTGAGAAGAAGATCCAAATTCACAA | |
| | CACGAGATATGGAGAACTGTTCACAAGGCTGTGTACGCATATAGAGAA | |
| | GAAACTGCTGGGGTCATCTTGGTCTAACAATGTCCCCCGGTCAGAGGAG | |
| | TTCAGCAGCATTCGTACGGATCCGGCATTCTGGTTTCACTCAAAATGGT | |
| | CCACAGCCAAGTTTGCATGGCTCCATATAAAACAGATCCAGAGGCATCT | |
| | GATGGTGGCAGCTAGGACAAGGTCTGCGGCCAACAAATTGGTGATGCT | |
| | AACCCATAAGGTAGGCCAAGTCTTTGTCACTCCTGAACTTGTCGTTGTG | |
| | ACGCATACGAATGAGAACAAGTTCACATGTCTTACCCAGGAACTTGTAT | |
| | TGATGTATGCAGATATGATGGAGGGCAGAGATATGGTCAACATAATATC | |
| | AACCACGGCGGTGCATCTCAGAAGCTTATCAGAGAAAATTGATGACATT | |
| | TTGCGGTTAATAGACGCTCTGGCAAAAGACTTGGGTAATCAAGTCTACG | |
| | ATGTTGTATCACTAATGGAGGGATTTGCATACGGAGCTGTCCAGCTACT | |
| | CGAGCCGTCAGGTACATTTGCAGGAGATTTCTTCGCATTCAACCTGCAG | |
| | GAGCTTAAAGACATTCTAATTGGCCTCCTCCCCAATGATATAGCAGAAT | |
| | CCGTGACTCATGCAATCGCTACTGTATTCTCTGGTTTAGAACAGAATCA | |
| | AGCAGCTGAGATGTTGTGTCTGTTGCGTCTGTGGGGTCACCCACTGCTT | |
| | GAGTCCCGTATTGCAGCAAAGGCAGTCAGGAGCCAAATGTGCGCACCG | |
| | AAAATGGTAGACTTTGATATGATCCTTCAGGTACTGTCTTTCTTCAAGGG | |
| | AACAATCATCAACGGGTACAGAAAGAAGAATGCAGGTGTGTGGCCGCG | |
| | AGTCAAAGTGGATACAATATATGGGAAGGTCATTGGGCAACTACATGC | |
| | AGATTCAGCAGAGATTTCACACGATATCATGTTGAGAGAGTATAAGAGT | |
| | TTATCTGCACTTGAATTTGAGCCATGTATAGAATATGACCCTGTCACCA | |
| | ACCTGAGCATGTTCCTAAAAGACAAGGCAATCGCACACCCCAACGATA | |
| | ATTGGCTTGCCTCGTTTAGGCGGAACCTTCTCTCCGAAGACCAGAAGAA | |
| | ACATGTAAAAGAAGCAACTTCGACTAATCGCCTCTTGATAGAGTTTTTA | |
| | GAGTCAAATGATTTTGATCCATATAAAGAGATGGAATATCTGACGACCC | |
| | TTGAGTACCTTAGAGATGACAATGTGGCAGTATCATACTCGCTCAAGGA | |
| | GAAGGAAGTGAAAGTTAATGGACGGATCTTCGCTAAGCTGACAAAGAA | |
| | GTTAAGGAACTGTCAGGTGATGGCGGAAGGGATCCTAGCCGATCAGAT | |
| | TGCACCTTTCTTTCAGGGAAATGGAGTCATTCAGGATAGCATATCCTTG | |
| | ACCAAGAGTATGCTAGCGATGAGTCAACTGTCTTTTAACAGCAATAAGA | |
| | AACGTATCACTGACTGTAAAGAAAGAGTATCTTCAAACCGCAATCATGA | |
| | TCCGAAAAGCAAGAACCGTCGGAGAGTTGCAACCTTCATAACAACTGA | |
| | CCTGCAAAAGTACTGTCTTAATTGGAGATATCAGACAATCAAATTGTTC | |
| | GCTCATGCCATCAATCAGTTGATGGGCCTACCTCACTTCTTCGAATGGAT | |
| | TCACCTAAGACTGATGGACACTACGATGTTCGTAGGAGACCCTTTCAAT | |
| | CCTCCAAGTGACCCTACTGACTGTGACCTCTCAAGAGTCCCTAATGATG | |
| | ACATATATATTGTCAGTGCCAGAGGGGGTATCGAAGGATTATGCCAGAA | |
| | GCTATGGACAATGATCTCAATTGCTGCAATCCAACTTGCTGCAGCTAGA | |
| | TCGCATTGTCGTGTTGCCTGTATGGTACAGGGTGATAATCAAGTAATAG | |
| | CAGTAACGAGAGAGGTAAGATCAGACGACTCTCCGGAGATGGTGTTGA | |
| | CACAGTTGCATCAAGCCAGTGATAATTTCTTCAAGGAATTAATTCATGT | |
| | CAATCATTTGATTGGCCATAATTTGAAGGATCGTGAAACCATCAGGTCA | |
| | GACACATTCTTCATATACAGCAAACGAATCTTCAAAGATGGAGCAATCC | |
| | TCAGTCAAGTCCTCAAAAATTCATCTAAATTAGTGCTAGTGTCAGGTGA | |
| | TCTCAGTGAAAACACCGTAATGTCCTGTGCCAACATTGCCTCTACTGTA | |
| | GCACGGCTATGCGAGAACGGGCTTCCCAAAGACTTCTGTTACTATTTAA | |
| | ACTATATAATGAGTTGTGTGCAGACATACTTTGACTCTGAGTTCTCCATC | |
| | ACCAACAATTCGCACCCCGATCTTAATCAGTCGTGGATTGAAGACATCT | |
| | CTTTTGTGCACTCATATGTTCTGACTCCTGCCCAATTAGGGGGACTGAGT | |
| | AACCTTCAATACTCAAGGCTCTACACTAGAAATATCGGTGACCCGGGGA | |
| | CTACTGCTTTTGCAGAGATCAAGCGACTAGAAGCAGTGGGATTACTGAG | |
| | TCCTAACATTATGACTAATATCTTAACTAGGCCGCCTGGGAATGGAGAT | |
| | TGGGCCAGTCTGTGCAACGACCCATACTCTTTCAATTTTGAGACTGTTGC | |
| | AAGCCCAAATATTGTTCTTAAGAAACATACGCAAAGAGTCCTATTTGAA | |
| | ACTTGTTCAAATCCCTTATTGTCTGGAGTGCACACAGAGGATAATGAGG | |
| | CAGAAGAGAAGGCATTGGCTGAATTCTTGCTTAATCAAGAGGTGATTCA | |
| | TCCCCGCGTTGCGCATGCCATCATGGAGGCAAGCTCTGTAGGTAGGAGA | |
| | AAGCAAATTCAAGGGCTTGTTGACACAACAAACACCGTAATTAAGATTG | |
| | CGCTTACTAGGAGGCCATTAGGCATCAAGAGGCTGATGCGGATAGTCA | |
| | ATTATTCTAGCATGCATGCAATGCTGTTTAGAGACGATGTTTTTTCCTCC | |
| | AGTAGATCCAACCACCCCTTAGTCTCTTCTAATATGTGTTCTCTGACACT | |
| | GGCAGACTATGCACGGAATAGAAGCTGGTCACCTTTGACGGGAGGCAG | |
| | GAAAATACTGGGTGTATCTAATCCTGATACGATAGAACTCGTAGAGGGT | |
| | GAGATTCTTAGTGTAAGCGGAGGGTGTACAAGATGTGACAGCGGAGAT | |
| | GAACAATTTACTTGGTTCCATCTTCCAAGCAATATAGAATTGACCGATG | |
| | ACACCAGCAAGAATCCTCCGATGAGGGTACCATATCTCGGGTCAAAGA | |
| | CACAGGAGAGGAGAGCTGCCTCACTTGCAAAAATAGCTCATATGTCGCC | |
| | ACATGTAAAGGCTGCCCTAAGGGCATCATCCGTGTTGATCTGGGCTTAT | |
| | GGGGATAATGAAGTAAATTGGACTGCTGCTCTTACGATTGCAAAATCTC | |
| | GGTGTAATGTAAACTTAGAGTATCTTCGGTTACTGTCCCCTTTACCCACG | |
| | GCTGGGAATCTTCAACATAGACTAGATGATGGTATAACTCAGATGACAT | |
| | TCACCCCTGCATCTCTCTACAGGGTGTCACCTTACATTCACATATCCAAT | |
| | GATTCTCAAAGGCTGTTCACTGAAGAAGGAGTCAAAGAGGGGAATGTG | |
| | GTTTACCAACAGATCATGCTCTTGGGTTTATCTCTAATCGAATCGATCTT | |
| | TCCAATGACAACAACCAGGACATATGATGAGATCACACTGCACCTACAT | |
| | AGTAAATTTAGTTGCTGTATCAGAGAAGCACCTGTTGCGGTTCCTTTCG | |
| | AGCTACTTGGGGTGGTACCGGAACTGAGGACAGTGACCTCAAATAAGTT | |
| | TATGTATGATCCTAGCCCTGTATCGGAGGGAGACTTTGCGAGACTTGAC | |
| | TTAGCTATCTTCAAGAGTTATGAGCTCAATCTGGAGTCATATCCCACGA | |
| | TAGAGCTAATGAACATTCTTTCAATATCCAGCGGGAAGTTGATTGGCCA | |
| | GTCTGTGGTTTCTTATGATGAAGATACCTCCATAAAGAATGACGCCATA | |
| | ATAGTGTATGACAATACCCGAAATTGGATCAGTGAAGCTCAGAATTCAG | |
| | ATGTGGTCCGCCTATTTGAATATGCAGCACTTGAAGTGCTCCTCGACTGT | |
| | TCTTACCAACTCTATTACCTGAGAGTAAGAGGCCTAGACAATATTGTCT | |
| | TATATATGGGTGATTTATACAAGAATATGCCAGGAATTCTACTTTCCAA | |
| | CATTGCAGCTACAATATCTCATCCCGTCATTCATTCAAGGTTACATGCAG | |
| | TGGGCCTGGTCAACCATGACGGATCACACCAACTTGCAGATACGGATTT | |
| | TATCGAAATGTCTGCAAAACTATTAGTATCTTGCACCCGACGTGTGATC | |
| | TCCGGCTTATATTCAGGAAATAAGTATGATCTGCTGTTCCCATCTGTCTT | |
| | AGATGATAACCTGAATGAGAAGATGCTTCAGCTGATATCCCGGTTATGC | |
| | TGTCTGTACACGGTACTCTTTGCTACAACAAGAGAAATCCCGAAAATAA | |
| | GAGGCTTAACTGCAGAAGAGAAATGTTCAATACTCACTGAGTATTTACT | |
| | GTCGGATGCTGTGAAACCATTACTTAGCCCCGATCAAGTGAGCTCTATC | |
| | ATGTCTCCTAACATAATTACATTCCCAGCTAATCTGTACTACATGTCTCG | |
| | GAAGAGCCTCAATTTGATCAGGGAAAGGGAGGACAGGGATACTATCCT | |
| | GGCGTTGTTGTTCCCCCAAGAGCCATTATTAGAGTTCCCTTCTGTGCAAG | |
| | ATATTGGTGCTCGAGTGAAAGATCCATTCACCCGACAACCTGCGGCATT | |
| | TTTGCAAGAGTTAGATTTGAGTGCTCCAGCAAGGTATGACGCATTCACA | |
| | CTTAGTCAGATTCATCCTGAACTCACATCTCCAAATCCGGAGGAAGACC | |
| | ACTTAGTACGATACTTGTTCAGAGGGATAGGGACTGCATCTTCCTCTTG | |
| | GTATAAGGCATCTCATCTCCTTTCTGTACCCGAGGTAAGATGTGCAAGA | |
| | CACGGGAACTCCTTATACTTAGCTGAAGGGAGCGGAGCCATCATGAGTC | |
| | TTCTCGAACTGCATGTACCACATGAAACTATCTATTACAATACGCTCTTT | |
| | TCAAATGAGATGAACCCCCCGCAACGACATTTCGGGCCGACCCCAACTC | |
| | AGTTTTTGAATTCGGTTGTTTATAGGAATCTACAGGCGGAGGTAACATG | |
| | CAAAGATGGATTTGTCCAAGAGTTCCGTCCATTATGGAGAGAAAATACA | |
| | GAGGAAAGTGACCTGACCTCAGATAAAGCAGTGGGGTATATTACATCT | |
| | GCAGTGCCCTACAGATCTGTATCATTGCTGCATTGTGACATTGAAATTCC | |
| | TCCAGGGTCCAATCAAAGCTTACTAGATCAACTAGCTATCAATTTATCT | |
| | CTGATTGCCATGCATTCTGTAAGGGAGGGCGGGGTAGTAATCATCAAAG | |
| | TGTTGTATGCAATGGGATACTACTTTCATCTACTCATGAACTTGTTTGCT | |
| | CCGTGTTCCACAAAAGGATATATTCTCTCTAATGGTTATGCATGTCGAG | |
| | GAGATATGGAGTGTTACCTGGTATTTGTCATGGGTTACCTGGGCGGGCC | |
| | TACATTTGTACATGAGGTGGTGAGGATGGCAAAAACTCTGGTGCAGCGG | |
| | CACGGTACGCTTTTGTCTAAATCAGATGAGATCACACTGACCAGGTTAT | |
| | TCACCTCACAGCGGCAGCGTGTGACAGACATCCTATCCAGTCCTTTACC | |
| | AAGATTAATAAAGTACTTGAGGAAGAAATTGACACTGCGCTGATTGAA | |
| | GCCGGGGGACAGCCCGTCCGTCCATTCTGTGCGGAGAGTCTGGTGAGCA | |
| | CGCTAGCGAACATAACTCAGATAACCCAGATCATCGCTAGCCACATTGA | |
| | CACAGTTATCCGGTCTGTGATATATATGGAAGCTGAGGGTGATCTCGCT | |
| | GACACAGTATTTCTATTTACCCCTTACAATCTCTCTACTGACGGGAAAA | |
| | AGAGGACATCACTTAAACAGTGCACGAGACAGATCCTAGAGGTTACAA | |
| | TACTAGGTCTTAGAGTCGAAAATCTCAATAAAATAGGCGATATAATCAG | |
| | CCTAGTGCTTAAAGGCATGATCTCCATGGAGGACCTTATCCCACTAAGG | |
| | ACATACTTGAAGCATAGTACCTGCCCTAAATATTTGAAGGCTGTCCTAG | |
| | GTATTACCAAACTCAAAGAAATGTTTACAGACACTTCTGTACTGTACTT | |
| | GACTCGTGCTCAACAAAAATTCTACATGAAAACTATAGGCAATGCAGTC | |
| | AAAGGATATTACAGTAACTGTGACTCTTAACGAAAATCACATATTAATA | |
| | GGCTCCTTTTTTGGCCAATTGTATTCTTGTTGATTTAATCATATTATGTTA | |
| | GAAAAAAGTTGAACCCTGACTCCTTAGGACTCGAATTCGAACTCAAATA | |
| | AATGTCTTAAAAAAAGGTTGCGCACAATTATTCTTGAGTGTAGTCTCGT | |
| | CATTCACCAAATCTTTGTTTGGT | |
| Sequence | accaaacagagaatccgtgagttacgataaaaggcgaaggagcaattgaagtcgcacgggtagaaggt | 84 |
| NDV LaSota | gtgaatctcgagtgcgagcccgaagcacaaactcgagaaagccttctgccaacatgtcttccgtatttgat | |
| L289A | gagtacgaacagctcctcgcggctcagactcgccccaatggagctcatggagggggagaaaaagggagt | |
| genome | accttaaaagtagacgtcccggtattcactcttaacagtgatgacccagaagatagatggagctttgtggt | |
| (Mutation | attctgcctccggattgctgttagcgaagatgccaacaaaccactcaggcaaggtgctctcatatctctttt | |
| L289A in the | atgctcccactcacaggtaatgaggaaccatgttgccCttgcagggaaacagaatgaagccacattggcc | |
| F protein. | gtgcttgagattgatggctttgccaacggcacgccccagttcaacaataggagtggagtgtctgaagaga | |
| CTA = Leu | gagcacagagatttgcgatgatagcaggatctctccctcgggcatgcagcaacggaaccccgttcgtcac | |
| changed to | agccggggcCgaagatgatgcaccagaagacatcaccgataccctggagaggatcctctctatccaggc | |
| GCC = Ala | tcaagtatgggtcacagtagcaaaagccatgactgcgtatgagactgcagatgagtcggaaacaaggcg | |
| (underlined | aatcaataagtatatgcagcaaggcagggtccaaaagaaatacatcctctaccccgtatgcaggagcac | |
| and bold). | aatccaactcacgatcagacagtctcttgcagtccgcatctttttggttagcgagctcaagagaggccgca | |
| Unique | acacggcaggtggtacctctacttattataacctggtaggggacgtagactcatacatcaggaataccgg | |
| restriction | gcttactgcattcttcttgacactcaagtacggaatcaacaccaagacatcagcccttgcacttagtagcct | |
| site Sac II | ctcaggcgacatccagaagatgaagcagctcatgcgtttgtatcggatgaaaggagataatgcgccgtac | |
| (CCGCGG) | atgacattacttggtgatagtgaccagatgagctttgcgcctgccgagtatgcacaactttactcctttgcc | |
| for insertion | atgggtatggcatcagtcctagataaaggtactgggaaataccaatttgccagggactttatgagcacatc | |
| of foreign | attctggagacttggagtagagtacgctcaggctcagggaagtagcattaacgaggatatggctgccgag | |
| genes double | ctaaagctaaccccagcagcaaGgaGgggcctggcagctgctgcccaacgggtctccgaGgaGacca | |
| underlined.) | gcagcataGacatgcctactcaacaagtcggagtcctcactgggcttagcgagggggggtcccaagctct | |
| | acaaggcggatcgaatagatcgcaagggcaaccagaagccggggatggggagacccaattcctggatc | |
| | tgatgagagcggtagcaaatagcatgagggaggcgccaaactctgcacagggcactccccaatcggggc | |
| | ctcccccaactcctgggccatcccaagataacgacaccgactgggggtattgatggacaaaacccagcct | |
| | gcttccacaaaaacatcccaatgccctcacccgtagtcgacccctcgatttgcggctctatatgaccacacc | |
| | ctcaaacaaacatccccctctttcctccctccccctgctgtacaactAcgTacgccctagataccacaggc | |
| | acaatgcggctcactaacaatcaaaacagagccgagggaattagaaaaaagtacgggtagaagaggg | |
| | atattcagagatcagggcaagtctcccgagtctctgctctctcctctacctgatagaccaggacaaacatg | |
| | gccacctttacagatgcagagatcgacgagctatttgagacaagtggaactgtcattgacaacataattac | |
| | agcccagggtaaaccagcagagactgttggaaggagtgcaatcccacaaggcaagaccaaggtgctga | |
| | gcgcagcatgggagaagcatgggagcatccagccaccggccagtcaagacaaccccgatcgacaggac | |
| | agatctgacaaacaaccatccacacccgagcaaacgaccccgcatgacagcccgccggccacatccgcc | |
| | gaccagccccccacccaggccacagacgaagccgtcgacacacagCtcaggaccggagcaagcaactc | |
| | tctgctgttgatgcttgacaagctcagcaataaatcgtccaatgctaaaaagggcccatggtcgagccccc | |
| | aagaggggaatcaccaacgtccgactcaacagcaggggagtcaacccagtcgcggaaacagtcaggaa | |
| | agaccgcagaaccaagtcaaggccgcccctggaaaccagggcacagacgtgaacacagcatatcatgg | |
| | acaatgggaggagtcacaactatcagctggtgcaacccctcatgctctccgatcaaggcagagccaaga | |
| | caatacccttgtatctgcggatcatgtccagccacctgtagactttgtgcaagcgatgatgtctatgatgga | |
| | ggcgatatcacagagagtaagtaaggttgactatcagctagatcttgtcttgaaacagacatcctccatcc | |
| | ctatgatgcggtccgaaatccaacagctgaaaacatctgttgcagtcatggaagccaacttgggaatgat | |
| | gaagattctggatcccggttgtgccaacatttcatctctgagtgatctacgggcagttgcccgatctcaccc | |
| | ggttttagtttcaggccctggagacccctctccctatgtgacacaaggaggcgaaatggcacttaataaac | |
| | tttcgcaaccagtgccacatccatctgaattgattaaacccgccactgcatgcgggcctgatataggagtg | |
| | gaaaaggacactgtccgtgcattgatcatgtcacgcccaatgcacccgagttcttcagccaagctcctaag | |
| | caagttagatgcagccgggtcgatcgaggaaatcaggaaaatcaagcgccttgctctaaatggctaatta | |
| | ctactgccacacgtagcgggtccctgtccactcggcatcacacggaatctgcaccgagttcccccccgcGg | |
| | acccaaggtccaactctccaagcggcaatcctctctcgcttcctcagccccactgaatgAtcgcgtaaccg | |
| | taattaatctagctacatttaagattaagaaaaaatacgggtagaattggagtgccccaattgtgccaaga | |
| | tggactgatagtaaggaggactcagtattcatcaccacctatggattcatctttcaagttgggaatggaga | |
| | gtcctacaagAcacaggagatgggaagaagcaaatcgccccgcaatataggatccagcgccttgacttg | |
| | tggactgatagtaaggaggactcagtattcatcaccacctatggattcatctttcaagttgggaatgaaga | |
| | agccacCgtcggcatgatcgatgataaacccaagcgcgagttactttccgctgcgatgctctgcctagga | |
| | agcgtcccaaataccggagaccttattgagctggcaagggcctgtctcactatgatagtcacatgcaaga | |
| | agagtgcaactaatactgagagaatggttttctcagtagtgcaggcaccccaagtgctgcaaagctgtag | |
| | ggttgtggcaaacaaatactcatcagtgaatgcagtcaagcacgtgaaagcgccagagaagattcccgg | |
| | gagtggaaccctagaatacaaggtgaactttgtctccttgactgtggtaccgaagaGggatgtctacaag | |
| | atcccagctgcagtattgaaggtttctggctcgagtctgtacaatcttgcgctcaatgtcactattaatgtgg | |
| | aggtagacccgaggagtcctttggttaaatctCtgtctaagtctgacagcggatactatgctaacctcttct | |
| | tgcatattggacttatgaccacTgtagataggaaggggaagaaagtgacatttgacaagctggaaaaga | |
| | aaataaggagccttgatctatctgtcgggctcagtgatgtgctcgggccttccgtgttggtaaaagcaaga | |
| | ggtgcacggactaagcttttggcacctttcttctctagcagtgggacagcctgctatcccatagcaaatgct | |
| | tctcctcaggtggccaagatactctggagtcaaaccgcgtgcctgcggagcgttaaaatcattatccaagc | |
| | aggtacccaacgcgctgtcgcagtgaccgccgaccacgaggttacctctactaagctggagaaggggca | |
| | cacccttgccaaatacaatccttttaagaaataagctgcgtctctgagattgcgctccgcccactcacccag | |
| | atcatcatgacacaaaaaactaatctgtcttgattatttacagttagtttacctgtctatcaagttagaaaa | |
| | aacacgggtagaagattctggatcccggttggcgccctccaggtgcaagatgggctccagaccttctacc | |
| | aagaacccagcacctatgatgctgactatccgggttgcgctggtactgagttgcatctgtccggcaaactc | |
| | cattgatggcaggcctcttgcagctgcaggaattgtggttacaggagacaaagccgtcaacatatacacct | |
| | catcccagacaggatcaatcatagttaagctcctcccgaatctgcccaaggataaggaggcatgtgcgaa | |
| | agcccccttggatgcatacaacaggacattgaccactttgctcaccccccttggtgactctatccgtaggat | |
| | acaagagtctgtgactacatctggaggggggagacaggggcgccttataggcgccattattggcggtgtg | |
| | gctcttggggttgcaactgccgcacaaataacagcggccgcagctctgatacaagccaaacaaaatgctg | |
| | ccaacatcctccgacttaaagagagcattgccgcaaccaatgaggctgtgcatgaggtcactgacggatt | |
| | atcgcaactagcagtggcagttgggaagatgcagcagtttgttaatgaccaatttaataaaacagctcag | |
| | gaattagactgcatcaaaattgcacagcaagttggtgtagagctcaacctgtacctaaccgaattgactac | |
| | agtattcggaccacaaatcacttcacctgctttaaacaagctgactattcaggcactttacaatctagctgg | |
| | tggaaatatggattacttattgactaagttaggtgtagggaacaatcaactcagctcattaatcggtagcg | |
| | gcttaatcaccggtaaccctattctatacgactcacagactcaactcttgggtatacaggtaactGCCcct | |
| | tcagtcgggaacctaaataatatgcgtgccacctacttggaaaccttatccgtaagcacaaccaggggatt | |
| | tgcctcggcacttgtcccAaaagtggtgacacaggtcggttctgtgatagaagaacttgacacctcatact | |
| | gtatagaaactgacttagatttatattgtacaagaatagtaacgttccctatgtcccctggtatttattcctg | |
| | cttgagcggcaatacgtcggcctgtatgtactcaaagaccgaaggcgcacttactacaccatacatgacta | |
| | tcaaaggttcagtcatcgccaactgcaagatgacaacatgtagatgtgtaaaccccccgggtatcatatcg | |
| | caaaactatggagaagccgtgtctctaatagataaacaatcatgcaatgttttatccttaggcgggataac | |
| | tttaaggctcagtggggaattcgatgtaacttatcagaagaatatctcaatacaagattctcaagtaataa | |
| | taacaggcaatcttgatatctcaactgagcttgggaatgtcaacaactcgatcagtaatgctttgaataagt | |
| | tagaggaaagcaacagaaaactagacaaagtcaatgtcaaactgactagcacatctgctctcattaccta | |
| | tatcgttttgactatcatatctcttgtttttggtatacttagcctgattctagcatgctacctaatgtacaag | |
| | caaaaggcgcaacaaaagaccttattatggcttgggaataatactctagatcagatgagagccactacaaa | |
| | aatgtgaacacagatgaggaacgaaggtttccctaatagtaatttgtgtgaaagttctggtagtctgtcag | |
| | ttcagagagttaagaaaaaactaccggttgtagatgaccaaaggacgatatacgggtagaacggtaaga | |
| | gaggccgcccctcaattgcgagccaggcttcacaacctccgttctaccgcttcaccgacaacagtcctcaa | |
| | tcatggaccgcgccgttagccaagttgcgttagagaatgatgaaagagaggcaaaaaatacatggcgctt | |
| | gatattccggattgcaatcttattcttaacagtagtgaccttggctatatctgtagcctcccttttatatag | |
| | catgggggctagcacacctagcgatcttgtaggcataccgactaggatttccagggcagaagaaaagattac | |
| | atctacacttggttccaatcaagatgtagtagataggatatataagcaagtggcccttgagtctccgttggc | |
| | attgttaaatactgagaccacaattatgaacgcaataacatctctctcttatcagattaatggagctgcaaa | |
| | caacagtgggtggggggcacctatccatgacccagattatataggggggataggcaaagaactcattgta | |
| | gatgatgctagtgatgtcacatcattctatccctctgcatttcaagaacatctgaattttatcccggcgccta | |
| | ctacaggatcaggttgcactcgaataccctcatttgacatgagtgctacccattactgctacacccataatg | |
| | taatattgtctggatgcagagatcactcacattcatatcagtatttagcacttggtgtgctccggacatctgc | |
| | aacagggagggtattcttttctactctgcgttccatcaacctggacgacacccaaaatcggaagtcttgca | |
| | gtgtgagtgcaactcccctgggttgtgatatgctgtgctcgaaagtcacggagacagaggaagaagatta | |
| | taactcagctgtccctacgcggatggtacatgggaggttagggttcgacggccagtaccacgaaaaggac | |
| | ctagatgtcacaacattattcggggactgggtggccaactacccaggagtagggggtggatcttttattga | |
| | cagccgcgtatggttctcagtctacggagggttaaaacccaattcacccagtgacactgtacaggaaggg | |
| | aaatatgtgatatacaagcgatacaatgacacatgcccagatgagcaagactaccagattcgaatggcc | |
| | aagtcttcgtataagcctggacggtttggtgggaaacgcatacagcaggctatcttatctatcaaggtgtc | |
| | aacatccttaggcgaagacccggtactgactgtaccgcccaacacagtcacactcatgggggccgaagg | |
| | cagaattctcacagtagggacatctcatttcttgtatcaacgagggtcatcatacttctctcccgcgttatta | |
| | tatcctatgacagtcagcaacaaaacagccactcttcatagtccttatacattcaatgccttcactcggcca | |
| | ggtagtatcccttgccaggcttcagcaagatgccccaactcgtgtgttactggagtctatacagatccatat | |
| | cccctaatcttctatagaaaccacaccttgcgaggggtattcgggacaatgcttgatggtgtacaagcaag | |
| | acttaaccctgcgtctgcagtattcgatagcacatcccgcagtcgcattactcgagtgagttcaagcagtac | |
| | caaagcagcatacacaacatcaacttgttttaaagtggtcaagactaataagacctattgtctcagcattg | |
| | ctgaaatatctaatactctcttcggagaattcagaatcgtcccgttactagttgagatcctcaaagatgacg | |
| | gggttagagaagccaggtctggctagttgagtcaattataaaggagttggaaagatggcattgtatcacct | |
| | atcttctgcgacatcaagaatcaaaccgaatgccggcgcgtgctcgaattccatgttgccagttgaccaca | |
| | atcagccagtgctcatgcgatcagattaagccttgtcaAtaGtctcttgattaagaaaaaatgtaagtggc | |
| | aatgagatacaaggcaaaacagctcatggtTaaCaatacgggtaggacatggcgagctccggtcctga | |
| | aagggcagagcatcagattatcctaccagagTcacacctgtcttcaccattggtcaagcacaaactactct | |
| | attactggaaattaactgggctaccgcttcctgatgaatgtgacttcgaccacctcattctcagccgacaat | |
| | ggaaaaaaatacttgaatcggcctctcctgatactgagagaatgataaaactcggaagggcagtacacc | |
| | aaactcttaaccacaattccagaataaccggagtgctccaccccaggtgtttagaaGaactggctaatatt | |
| | gaggtcccagattcaaccaacaaatttcggaagattgagaagaagatccaaattcacaacacgagatat | |
| | ggagaactgttcacaaggctgtgtacgcatatagagaagaaactgctggggtcatcttggtctaacaatgt | |
| | cccccggtcagaggagttcagcagcattcgtacggatccggcattctggtttcactcaaaatggtccacag | |
| | ccaagtttgcatggctccatataaaacagatccagaggcatctgatggtggcagctaGgacaaggtctgc | |
| | ggccaacaaattggtgatgctaacccataaggtaggccaagtctttgtcactcctgaacttgtcgttgtgac | |
| | gcatacgaatgagaacaagttcacatgtcttacccaggaacttgtattgatgtatgcagatatgatggagg | |
| | gcagagatatggtcaacataatatcaaccacggcggtgcatctcagaagcttatcagagaaaattgatga | |
| | cattttgcggttaatagacgctctggcaaaagacttgggtaatcaagtctacgatgttgtatcactaatgga | |
| | gggatttgcatacggagctgtccagctactcgagccgtcaggtacatttgcaggagatttcttcgcattcaa | |
| | cctgcaggagcttaaagacattctaattggcctcctccccaatgatatagcagaatccgtgactcatgcaa | |
| | tcgctactgtattctctggtttagaacagaatcaagcagctgagatgttgtgtctgttgcgtctgtggggtca | |
| | cccactgcttgagtcccgtattgcagcaaaggcagtcaggagccaaatgtgcgcaccgaaaatggtagac | |
| | tttgatatgatccttcaggtactgtctttcttcaagggaacaatcatcaacgggtacagaaagaagaatgc | |
| | aggtgtgtggccgcgagtcaaagtggatacaatatatgggaaggtcattgggcaactacatgcagattca | |
| | gcagagatttcacacgatatcatgttgagagagtataagagtttatctgcacttgaatttgagccatgtata | |
| | gaatatgaccctgtcaccaacctgagcatgttcctaaaagacaaggcaatcgcacaccccaacgataatt | |
| | ggcttgcctcgtttaggcggaaccttctctccgaagaccagaagaaacatgtaaaagaagcaacttcgac | |
| | taatcgcctcttgatagagtttttagagtcaaatgattttgatccatataaagagatggaatatctgacgac | |
| | ccttgagtaccttagagatgacaatgtggcagtatcatactcgctcaaggagaaggaagtgaaagttaat | |
| | ggacggatcttcgctaagctgacaaagaagttaaggaactgtcaggtgatggcggaagggatcctagcc | |
| | gatcagattgcacctttctttcagggaaatggagtcattcaggatagcatatccttgaccaagagtatgcta | |
| | gcgatgagtcaactgtcttttaacagcaataagaaacgtatcactgactgtaaagaaagagtatcttcaa | |
| | accgcaatcatgatccgaaaagcaagaaccgtcggagagttgcaaccttcataacaactgacctgcaaa | |
| | agtactgtcttaattggagatatcagacaatcaaattgttcgctcatgccatcaatcagttgatgggcctac | |
| | ctcacttcttcgaatggattcacctaagactgatggacactacgatgttcgtaggagaccctttcaatcctc | |
| | caagtgaccctactgactgtgacctctcaagagtccctaatgatgacatatatattgtcagtgccagaggg | |
| | ggtatcgaaggattatgccagaagctatggacaatgatctcaattgctgcaatccaacttgctgcagctag | |
| | atcgcattgtcgtgttgcctgtatggtacagggtgataatcaagtaatagcagtaacgagagaggtaagat | |
| | cagacgactctccggagatggtgttgacacagttgcatcaagccagtgataatttcttcaaggaattaatt | |
| | catgtcaatcatttgattggccataatttgaaggatcgtgaaaccatcaggtcagacacattcttcatatac | |
| | agcaaacgaatcttcaaagatggagcaatcctcagtcaagtcctcaaaaattcatctaaattagtgctagt | |
| | gtcaggtgatctcagtgaaaacaccgtaatgtcctgtgccaacattgcctctactgtagcacggctatgcg | |
| | agaacgggcttcccaaagacttctgttactatttaaactatataatgagttgtgtgcagacatactttgactc | |
| | tgagttctccatcaccaacaattcgcaccccgatcttaatcagtcgtggattgaggacatctcttttgtgcac | |
| | tcatatgttctgactcctgcccaattagggggactgagtaaccttcaatactcaaggctctacactagaaat | |
| | atcggtgacccggggactactgcttttgcagagatcaagcgactagaagcagtgggattactgagtccta | |
| | acattatgactaatatcttaactaggccgcctgggaatggagattgggccagtctgtgcaacgacccatac | |
| | tctttcaattttgagactgttgcaagcccaaatattgttcttaagaaacatacgcaaagagtcctatttgaa | |
| | acttgttcaaatcccttattgtctggagtgcacacagaggataatgaggcagaagagaaggcattggctg | |
| | aattcttgcttaatcaagaggtgattcatccccgcgttgcgcatgccatcatggaggcaagctctgtaggta | |
| | ggagaaagcaaattcaagggcttgttgacacaacaaacaccgtaattaagattgcgcttactaggaggcc | |
| | attaggcatcaagaggctgatgcggatagtcaattattctagcatgcatgcaatgctgtttagagacgatg | |
| | ttttttcctccagtagatccaaccaccccttagtctcttctaatatgtgttctctgacactggcagactatg | |
| | cacggaatagaagctggtcacctttgacgggaggcaggaaaatactgggtgtatctaatcctgatacgatag | |
| | aactcgtagagggtgagattcttagtgtaagcggagggtgtacaagatgtgacagcggagatgaacaatt | |
| | tacttggttccatcttccaagcaatatagaattgaccgatgacaccagcaagaatcctccgatgagggtac | |
| | catatctcgggtcaaagacacaggagaggagagctgcctcacttgcaaaaatagctcatatgtcgccaca | |
| | tgtaaaggctgccctaagggcatcatccgtgttgatctgggcttatggggataatgaagtaaattggactg | |
| | ctgctcttacgattgcaaaatctcggtgtaatgtaaacttagagtatcttcggttactgtcccctttacccac | |
| | ggctgggaatcttcaacatagactagatgatggtataactcagatgacattcacccctgcatctctctaca | |
| | ggGtgtcaccttacattcacatatccaatgattctcaaaggctgttcactgaagaaggagtcaaagaggg | |
| | gaatgtggtttaccaacagatcatgctcttgggtttatctctaatcgaatcgatctttccaatgacaacaacc | |
| | aggacatatgatgagatcacactgcacctacatagtaaatttagttgctgtatcagagaagcacctgttgc | |
| | ggttcctttcgagctacttggggtggtaccggaactgaggacagtgacctcaaataagtttatgtatgatcc | |
| | tagccctgtatcggagggagactttgcgagacttgacttagctatcttcaagagttatgagcttaatctgga | |
| | gtcatatcccacgatagagctaatgaacattctttcaatatccagcgggaagttgattggccagtctgtggt | |
| | ttcttatgatgaagatacctccataaagaatgacgccataatagtgtatgacaatacccgaaattggatca | |
| | gtgaagctcagaattcagatgtggtccgcctatttgaatatgcagcacttgaagtgctcctcgactgttctta | |
| | ccaactctattacctgagagtaagaggcctGgacaatattgtcttatatatgggtgatttatacaagaatat | |
| | gccaggaattctactttccaacattgcagctacaatatctcatcccgtcattcattcaaggttacatgcagtg | |
| | ggcctggtcaaccatgacggatcacaccaacttgcagatacggattttatcgaaatgtctgcaaaactatt | |
| | agtatcttgcacccgacgtgtgatctccggcttatattcaggaaataagtatgatctgctgttcccatctgtc | |
| | ttagatgataacctgaatgagaagatgcttcagctgatatcccggttatgctgtctgtacacggtactctttg | |
| | ctacaacaagagaaatcccgaaaataagaggcttaactgcagaagagaaatgttcaatactcactgagt | |
| | atttactgtcggatgctgtgaaaccattacttagccccgatcaagtgagctctatcatgtctcctaacataat | |
| | tacattcccagctaatctgtactacatgtctcggaagagcctcaatttgatcagggaaagggaggacagg | |
| | gatactatcctggcgttgttgttcccccaagagccattattagagttcccttctgtgcaagatattggtgctc | |
| | gagtgaaagatccattcacccgacaacctgcggcatttttgcaagagttagatttgagtgctccagcaagg | |
| | tatgacgcattcacacttagtcagattcatcctgaactcacatctccaaatccggaggaagactacttagta | |
| | cgatacttgttcagagggatagggactgcatcttcctcttggtataaggcatctcatctcctttctgtacccg | |
| | aggtaagatgtgcaagacacgggaactccttatacttagctgaagggagcggagccatcatgagtcttct | |
| | cgaactgcatgtaccacatgaaactatctattacaatacgctcttttcaaatgagatgaaccccccgcaac | |
| | gacatttcgggccgaccccaactcagtttttgaattcggttgtttataggaatctacaggcggaggtaacat | |
| | gcaaagatggatttgtccaagagttccgtccattatggagagaaaatacagaggaaagCgacctgacct | |
| | cagataaagTagtggggtatattacatctgcagtgccctacagatctgtatcattgctgcattgtgacattg | |
| | aaattcctccagggtccaatcaaagcttactagatcaactagctatcaatttatctctgattgccatgcattc | |
| | tgtaagggagggcggggtagtaatcatcaaagtgttgtatgcaatgggatactactttcatctactcatga | |
| | acttgtttgctccgtgttccacaaaaggatatattctctctaatggttatgcatgtcgaggagatatggagtg | |
| | ttacctggtatttgtcatgggttacctgggcgggcctacatttgtacatgaggtggtgaggatggcGaaaa | |
| | ctctggtgcagcggcacggtacgctTttgtctaaatcagatgagatcacactgaccaggttattcacctca | |
| | cagcggcagcgtgtgacagacatcctatccagtcctttaccaagattaataaagtacttgaggaagaatat | |
| | tgacactgcgctgattgaagccgggggacagcccgtccgtccattctgtgcggagagtctggtgagcacg | |
| | ctagcgaacataactcagataacccagatCatcgctagtcacattgacacagttatccggtctgtgatata | |
| | tatggaagctgagggtgatctcgctgacacagtatttctatttaccccttacaatctctctactgacgggaa | |
| | aaagaggacatcacttaAacagtgcacgagacagatcctagaggttacaatactaggtcttagagtcga | |
| | aaatctcaataaaataggcgatataatcagcctagtgcttaaaggcatgatctccatggaggaccttatcc | |
| | cactaaggacatacttgaagcatagtacctgccctaaatatttgaaggctgtcctaggtattaccaaactc | |
| | aaagaaatgtttacagacacttctgtaCtgtacttgactcgtgctcaacaaaaattctacatgaaaactat | |
| | aggcaatgcagtcaaaggatattacagtaactgtgactcttaacgaaaatcacatattaataggctccttt | |
| | tttggccaattgtattcttgttgatttaatcatattatgttagaaaaaagttgaaccctgactccttaggact | |
| | cgaattcgaactcaaataaatgtcttaaaaaaaggttgcgcacaattattcttgagtgtagtctcgtcattca | |
| | ccaaatctttgtttggt | |
| Plasmid | TCTTCCGCTTCCTCGCTCACTGACTCGCTGCGCTCGGTCGTTCGGCTGCGGCGAG | 85 |
| pNDV- | CGGTATCAGCTCACTCAAAGGCGGTAATACGGTTATCCACAGAATCAGGGGATA | |
| LaSotaL289 | ACGCAGGAAAGAACATGTGAGCAAAAGGCCAGCAAAAGGCCAGGAACCGTAA | |
| A | AAAGGCCGCGTTGCTGGCGTTTTTCCATAGGCTCCGCCCCCTGACGAGCATCAC | |
| (Viral | AAAAATCGACGCTCAAGTCAGAGGTGGCGAAACCCGACAGGACTATAAAGATA | |
| sequences in | CCAGGCGTTTCCCCCTGGAAGCTCCCTCGTGCGCTCTCCTGTTCCGACCCTGCCG | |
| bold. | CTTACCGGATACCTGTCCGCTTTCTCCCTTCGGGAAGCGTGGCGCTTTCTCATA | |
| Unique | GCTCACGCTGTAGGTATCTCAGTTCGGTGTAGGTCGTTCGCTCCAAGCTGGGCT | |
| restriction | GTGTGCACGAACCCCCCGTTCAGCCCGACCGCTGCGCCTTATCCGGTAACTATCG | |
| site Sac II | TCTTGAGTCCAACCCGGTAAGACACGACTTATCGCCACTGGCAGCAGCCACTGG | |
| (CCGCGG) | TAACAGGATTAGCAGAGCGAGGTATGTAGGCGGTGCTACAGAGTTCTTGAAGT | |
| for insertion | GGTGGCCTAACTACGGCTACACTAGAAGAACAGTATTTGGTATCTGCGCTCTGCT | |
| of foreign | GAAGCCAGTTACCTTCGGAAAAAGAGTTGGTAGCTCTTGATCCGGCAAACAAAC | |
| genes double | CACCGCTGGTAGCGGTGGTTTTTTTGTTTGCAAGCAGCAGATTACGCGCAGAAA | |
| underlined. | AAAAGGATCTCAAGAAGATCCTTTGATCTTTTCTACGGGGTCTGACGCTCAGTGG | |
| Mutation | AACGAAAACTCACGTTAAGGGATTTTGGTCATGAGATTATCAAAAAGGATCTTC | |
| L289A in the | ACCTAGATCCTTTTAAATTAAAAATGAAGTTTTAAATCAATCTAAAGTATATATGA | |
| F protein. | GTAAACTTGGTCTGACAGTTACCAATGCTTAATCAGTGAGGCACCTATCTCAGCG | |
| CTA = Leu | ATCTGTCTATTTCGTTCATCCATAGTTGCCTGACTCCCCGTCGTGTAGATAACTAC | |
| changed to | GATACGGGAGGGCTTACCATCTGGCCCCAGTGCTGCAATGATACCGCGAGACCC | |
| GCC = Ala | ACGCTCACCGGCTCCAGATTTATCAGCAATAAACCAGCCAGCCGGAAGGGCCGA | |
| (underlined | GCGCAGAAGTGGTCCTGCAACTTTATCCGCCTCCATCCAGTCTATTAATTGTTGC | |
| and | CGGGAAGCTAGAGTAAGTAGTTCGCCAGTTAATAGTTTGCGCAACGTTGTTGCC | |
| italicized).) | ATTGCTACAGGCATCGTGGTGTCACGCTCGTCGTTTGGTATGGCTTCATTCAGCT | |
| | CCGGTTCCCAACGATCAAGGCGAGTTACATGATCCCCCATGTTGTGCAAAAAAG | |
| | CGGTTAGCTCCTTCGGTCCTCCGATCGTTGTCAGAAGTAAGTTGGCCGCAGTGTT | |
| | ATCACTCATGGTTATGGCAGCACTGCATAATTCTCTTACTGTCATGCCATCCGTAA | |
| | GATGCTTTTCTGTGACTGGTGAGTACTCAACCAAGTCATTCTGAGAATAGTGTAT | |
| | GCGGCGACCGAGTTGCTCTTGCCCGGCGTCAATACGGGATAATACCGCGCCACA | |
| | TAGCAGAACTTTAAAAGTGCTCATCATTGGAAAACGTTCTTCGGGGCGAAAACT | |
| | CTCAAGGATCTTACCGCTGTTGAGATCCAGTTCGATGTAACCCACTCGTGCACCC | |
| | AACTGATCTTCAGCATCTTTTACTTTCACCAGCGTTTCTGGGTGAGCAAAAACAG | |
| | GAAGGCAAAATGCCGCAAAAAAGGGAATAAGGGCGACACGGAAATGTTGAAT | |
| | ACTCATACTCTTCCTTTTTCAATATTATTGAAGCATTTATCAGGGTTATTGTCTCAT | |
| | GAGCGGATACATATTTGAATGTATTTAGAAAAATAAACAAATAGGGGTTCCGCG | |
| | CACATTTCCCCGAAAAGTGCCACCTGACGTCTAAGAAACCATTATTATCATGACA | |
| | TTAACCTATAAAAATAGGCGTATCACGAGGCCCTTTCGTCTCGCGCGTTTCGGTG | |
| | ATGACGGTGAAAACCTCTGACACATGCAGCTCCCGGAGACGGTCACAGCTTGTC | |
| | TGTAAGCGGATGCCGGGAGCAGACAAGCCCGTCAGGGCGCGTCAGCGGGTGTT | |
| | GGCGGGTGTCGGGGCTGGCTTAACTATGCGGCATCAGAGCAGATTGTACTGAG | |
| | AGTGCACCATAAAATTGTAAACGTTAATATTTTGTTAAAATTCGCGTTAAATTTTT | |
| | GTTAAATCAGCTCATTTTTTAACCAATAGGCCGAAATCGGCAAAATCCCTTATAA | |
| | ATCAAAAGAATAGCCCGAGATAGGGTTGAGTGTTGTTCCAGTTTGGAACAAGAG | |
| | TCCACTATTAAAGAACGTGGACTCCAACGTCAAAGGGCGAAAAACCGTCTATCA | |
| | GGGCGATGGCCCACTACGTGAACCATCACCCAAATCAAGTTTTTTGGGGTCGAG | |
| | GTGCCGTAAAGCACTAAATCGGAACCCTAAAGGGAGCCCCCGATTTAGAGCTTG | |
| | ACGGGGAAAGCCGGCGAACGTGGCGAGAAAGGAAGGGAAGAAAGCGAAAGG | |
| | AGCGGGCGCTAGGGCGCTGGCAAGTGTAGCGGTCACGCTGCGCGTAACCACCA | |
| | CACCCGCCGCGCTTAATGCGCCGCTACAGGGCGCGTACTATGGTTGCTTTGACG | |
| | TATGCGGTGTGAAATACCGCACAGATGCGTAAGGAGAAAATACCGCATCAGGC | |
| | GCCATTCGCCATTCAGGCTGCGCAACTGTTGGGAAGGGCGATCGGTGCGGGCCT | |
| | CTTCGCTATTACGCCAGCTGGCGAAAGGGGGATGTGCTGCAAGGCGATTAAGTT | |
| | GGGTAACGCCAGGGTTTTCCCAGTCACGACGTTGTAAAACGACGGCCAGTGCCA | |
| | AGCTTTAATACGACTCACTATAGGGACCAAACAGAGAATCCGTGAGTTACGATA | |
| | AAAGGCGAAGGAGCAATTGAAGTCGCACGGGTAGAAGGTGTGAATCTCGAGT | |
| | GCGAGCCCGAAGCACAAACTCGAGAAAGCCTTCTGCCAACATGTCTTCCGTATT | |
| | TGATGAGTACGAACAGCTCCTCGCGGCTCAGACTCGCCCCAATGGAGCTCATG | |
| | GAGGGGGAGAAAAAGGGAGTACCTTAAAAGTAGACGTCCCGGTATTCACTCT | |
| | TAACAGTGATGACCCAGAAGATAGATGGAGCTTTGTGGTATTCTGCCTCCGGA | |
| | TTGCTGTTAGCGAAGATGCCAACAAACCACTCAGGCAAGGTGCTCTCATATCTC | |
| | TTTTATGCTCCCACTCACAGGTAATGAGGAACCATGTTGCCCTTGCAGGGAAAC | |
| | AGAATGAAGCCACATTGGCCGTGCTTGAGATTGATGGCTTTGCCAACGGCACG | |
| | CCCCAGTTCAACAATAGGAGTGGAGTGTCTGAAGAGAGAGCACAGAGATTTG | |
| | CGATGATAGCAGGATCTCTCCCTCGGGCATGCAGCAACGGAACCCCGTTCGTC | |
| | ACAGCCGGGGCCGAAGATGATGCACCAGAAGACATCACCGATACCCTGGAGA | |
| | GGATCCTCTCTATCCAGGCTCAAGTATGGGTCACAGTAGCAAAAGCCATGACT | |
| | GCGTATGAGACTGCAGATGAGTCGGAAACAAGGCGAATCAATAAGTATATGC | |
| | AGCAAGGCAGGGTCCAAAAGAAATACATCCTCTACCCCGTATGCAGGAGCACA | |
| | ATCCAACTCACGATCAGACAGTCTCTTGCAGTCCGCATCTTTTTGGTTAGCGAG | |
| | CTCAAGAGAGGCCGCAACACGGCAGGTGGTACCTCTACTTATTATAACCTGGT | |
| | AGGGGACGTAGACTCATACATCAGGAATACCGGGCTTACTGCATTCTTCTTGAC | |
| | ACTCAAGTACGGAATCAACACCAAGACATCAGCCCTTGCACTTAGTAGCCTCTC | |
| | AGGCGACATCCAGAAGATGAAGCAGCTCATGCGTTTGTATCGGATGAAAGGA | |
| | GATAATGCGCCGTACATGACATTACTTGGTGATAGTGACCAGATGAGCTTTGC | |
| | GCCTGCCGAGTATGCACAACTTTACTCCTTTGCCATGGGTATGGCATCAGTCCT | |
| | AGATAAAGGTACTGGGAAATACCAATTTGCCAGGGACTTTATGAGCACATCAT | |
| | TCTGGAGACTTGGAGTAGAGTACGCTCAGGCTCAGGGAAGTAGCATTAACGA | |
| | GGATATGGCTGCCGAGCTAAAGCTAACCCCAGCAGCAAGGAGGGGCCTGGCA | |
| | GCTGCTGCCCAACGGGTCTCCGAGGAGACCAGCAGCATAGACATGCCTACTCA | |
| | ACAAGTCGGAGTCCTCACTGGGCTTAGCGAGGGGGGGTCCCAAGCTCTACAAG | |
| | GCGGATCGAATAGATCGCAAGGGCAACCAGAAGCCGGGGATGGGGAGACCC | |
| | AATTCCTGGATCTGATGAGAGCGGTAGCAAATAGCATGAGGGAGGCGCCAAA | |
| | CTCTGCACAGGGCACTCCCCAATCGGGGCCTCCCCCAACTCCTGGGCCATCCCA | |
| | AGATAACGACACCGACTGGGGGTATTGATGGACAAAACCCAGCCTGCTTCCAC | |
| | AAAAACATCCCAATGCCCTCACCCGTAGTCGACCCCTCGATTTGCGGCTCTATAT | |
| | GACCACACCCTCAAACAAACATCCCCCTCTTTCCTCCCTCCCCCTGCTGTACAACT | |
| | ACGTACGCCCTAGATACCACAGGCACAATGCGGCTCACTAACAATCAAAACAG | |
| | AGCCGAGGGAATTAGAAAAAAGTACGGGTAGAAGAGGGATATTCAGAGATC | |
| | AGGGCAAGTCTCCCGAGTCTCTGCTCTCTCCTCTACCTGATAGACCAGGACAAA | |
| | CATGGCCACCTTTACAGATGCAGAGATCGACGAGCTATTTGAGACAAGTGGAA | |
| | CTGTCATTGACAACATAATTACAGCCCAGGGTAAACCAGCAGAGACTGTTGGA | |
| | AGGAGTGCAATCCCACAAGGCAAGACCAAGGTGCTGAGCGCAGCATGGGAGA | |
| | AGCATGGGAGCATCCAGCCACCGGCCAGTCAAGACAACCCCGATCGACAGGA | |
| | CAGATCTGACAAACAACCATCCACACCCGAGCAAACGACCCCGCATGACAGCC | |
| | CGCCGGCCACATCCGCCGACCAGCCCCCCACCCAGGCCACAGACGAAGCCGTC | |
| | GACACACAGCTCAGGACCGGAGCAAGCAACTCTCTGCTGTTGATGCTTGACAA | |
| | GCTCAGCAATAAATCGTCCAATGCTAAAAAGGGCCCATGGTCGAGCCCCCAAG | |
| | AGGGGAATCACCAACGTCCGACTCAACAGCAGGGGAGTCAACCCAGTCGCGG | |
| | AAACAGTCAGGAAAGACCGCAGAACCAAGTCAAGGCCGCCCCTGGAAACCAG | |
| | GGCACAGACGTGAACACAGCATATCATGGACAATGGGAGGAGTCACAACTAT | |
| | CAGCTGGTGCAACCCCTCATGCTCTCCGATCAAGGCAGAGCCAAGACAATACC | |
| | CTTGTATCTGCGGATCATGTCCAGCCACCTGTAGACTTTGTGCAAGCGATGATG | |
| | TCTATGATGGAGGCGATATCACAGAGAGTAAGTAAGGTTGACTATCAGCTAGA | |
| | TCTTGTCTTGAAACAGACATCCTCCATCCCTATGATGCGGTCCGAAATCCAACA | |
| | GCTGAAAACATCTGTTGCAGTCATGGAAGCCAACTTGGGAATGATGAAGATTC | |
| | TGGATCCCGGTTGTGCCAACATTTCATCTCTGAGTGATCTACGGGCAGTTGCCC | |
| | GATCTCACCCGGTTTTAGTTTCAGGCCCTGGAGACCCCTCTCCCTATGTGACACA | |
| | AGGAGGCGAAATGGCACTTAATAAACTTTCGCAACCAGTGCCACATCCATCTG | |
| | AATTGATTAAACCCGCCACTGCATGCGGGCCTGATATAGGAGTGGAAAAGGA | |
| | CACTGTCCGTGCATTGATCATGTCACGCCCAATGCACCCGAGTTCTTCAGCCAA | |
| | GCTCCTAAGCAAGTTAGATGCAGCCGGGTCGATCGAGGAAATCAGGAAAATC | |
| | AAGCGCCTTGCTCTAAATGGCTAATTACTACTGCCACACGTAGCGGGTCCCTGT | |
| | CCACTCGGCATCACACGGAATCTGCACCGAGTTCCCCCCCGCGGACCCAAGGTC | |
| | CAACTCTCCAAGCGGCAATCCTCTCTCGCTTCCTCAGCCCCACTGAATGATCGCG | |
| | TAACCGTAATTAATCTAGCTACATTTAAGATTAAGAAAAAATACGGGTAGAAT | |
| | TGGAGTGCCCCAATTGTGCCAAGATGGACTCATCTAGGACAATTGGGCTGTAC | |
| | TTTGATTCTGCCCATTCTTCTAGCAACCTGTTAGCATTTCCGATCGTCCTACAAG | |
| | ACACAGGAGATGGGAAGAAGCAAATCGCCCCGCAATATAGGATCCAGCGCCT | |
| | TGACTTGTGGACTGATAGTAAGGAGGACTCAGTATTCATCACCACCTATGGATT | |
| | CATCTTTCAAGTTGGGAATGAAGAAGCCACCGTCGGCATGATCGATGATAAAC | |
| | CCAAGCGCGAGTTACTTTCCGCTGCGATGCTCTGCCTAGGAAGCGTCCCAAATA | |
| | CCGGAGACCTTATTGAGCTGGCAAGGGCCTGTCTCACTATGATAGTCACATGC | |
| | AAGAAGAGTGCAACTAATACTGAGAGAATGGTTTTCTCAGTAGTGCAGGCACC | |
| | CCAAGTGCTGCAAAGCTGTAGGGTTGTGGCAAACAAATACTCATCAGTGAATG | |
| | CAGTCAAGCACGTGAAAGCGCCAGAGAAGATTCCCGGGAGTGGAACCCTAGA | |
| | ATACAAGGTGAACTTTGTCTCCTTGACTGTGGTACCGAAGAGGGATGTCTACA | |
| | AGATCCCAGCTGCAGTATTGAAGGTTTCTGGCTCGAGTCTGTACAATCTTGCGC | |
| | TCAATGTCACTATTAATGTGGAGGTAGACCCGAGGAGTCCTTTGGTTAAATCTC | |
| | TGTCTAAGTCTGACAGCGGATACTATGCTAACCTCTTCTTGCATATTGGACTTAT | |
| | GACCACTGTAGATAGGAAGGGGAAGAAAGTGACATTTGACAAGCTGGAAAA | |
| | GAAAATAAGGAGCCTTGATCTATCTGTCGGGCTCAGTGATGTGCTCGGGCCTT | |
| | CCGTGTTGGTAAAAGCAAGAGGTGCACGGACTAAGCTTTTGGCACCTTTCTTCT | |
| | CTAGCAGTGGGACAGCCTGCTATCCCATAGCAAATGCTTCTCCTCAGGTGGCCA | |
| | AGATACTCTGGAGTCAAACCGCGTGCCTGCGGAGCGTTAAAATCATTATCCAA | |
| | GCAGGTACCCAACGCGCTGTCGCAGTGACCGCCGACCACGAGGTTACCTCTAC | |
| | TAAGCTGGAGAAGGGGCACACCCTTGCCAAATACAATCCTTTTAAGAAATAAG | |
| | CTGCGTCTCTGAGATTGCGCTCCGCCCACTCACCCAGATCATCATGACACAAAA | |
| | AACTAATCTGTCTTGATTATTTACAGTTAGTTTACCTGTCTATCAAGTTAGAAAA | |
| | AACACGGGTAGAAGATTCTGGATCCCGGTTGGCGCCCTCCAGGTGCAAGATGG | |
| | GCTCCAGACCTTCTACCAAGAACCCAGCACCTATGATGCTGACTATCCGGGTTG | |
| | CGCTGGTACTGAGTTGCATCTGTCCGGCAAACTCCATTGATGGCAGGCCTCTTG | |
| | CAGCTGCAGGAATTGTGGTTACAGGAGACAAAGCCGTCAACATATACACCTCA | |
| | TCCCAGACAGGATCAATCATAGTTAAGCTCCTCCCGAATCTGCCCAAGGATAAG | |
| | GAGGCATGTGCGAAAGCCCCCTTGGATGCATACAACAGGACATTGACCACTTT | |
| | GCTCACCCCCCTTGGTGACTCTATCCGTAGGATACAAGAGTCTGTGACTACATC | |
| | TGGAGGGGGGAGACAGGGGCGCCTTATAGGCGCCATTATTGGCGGTGTGGCT | |
| | CTTGGGGTTGCAACTGCCGCACAAATAACAGCGGCCGCAGCTCTGATACAAGC | |
| | CAAACAAAATGCTGCCAACATCCTCCGACTTAAAGAGAGCATTGCCGCAACCA | |
| | ATGAGGCTGTGCATGAGGTCACTGACGGATTATCGCAACTAGCAGTGGCAGTT | |
| | GGGAAGATGCAGCAGTTTGTTAATGACCAATTTAATAAAACAGCTCAGGAATT | |
| | AGACTGCATCAAAATTGCACAGCAAGTTGGTGTAGAGCTCAACCTGTACCTAA | |
| | CCGAATTGACTACAGTATTCGGACCACAAATCACTTCACCTGCTTTAAACAAGC | |
| | TGACTATTCAGGCACTTTACAATCTAGCTGGTGGAAATATGGATTACTTATTGA | |
| | CTAAGTTAGGTGTAGGGAACAATCAACTCAGCTCATTAATCGGTAGCGGCTTA | |
| | ATCACCGGTAACCCTATTCTATACGACTCACAGACTCAACTCTTGGGTATACAG | |
| | GTAACTGCCCCTTCAGTCGGGAACCTAAATAATATGCGTGCCACCTACTTGGAA | |
| | ACCTTATCCGTAAGCACAACCAGGGGATTTGCCTCGGCACTTGTCCCAAAAGTG | |
| | GTGACACAGGTCGGTTCTGTGATAGAAGAACTTGACACCTCATACTGTATAGA | |
| | AACTGACTTAGATTTATATTGTACAAGAATAGTAACGTTCCCTATGTCCCCTGG | |
| | TATTTATTCCTGCTTGAGCGGCAATACGTCGGCCTGTATGTACTCAAAGACCGA | |
| | AGGCGCACTTACTACACCATACATGACTATCAAAGGTTCAGTCATCGCCAACTG | |
| | CAAGATGACAACATGTAGATGTGTAAACCCCCCGGGTATCATATCGCAAAACT | |
| | ATGGAGAAGCCGTGTCTCTAATAGATAAACAATCATGCAATGTTTTATCCTTAG | |
| | GCGGGATAACTTTAAGGCTCAGTGGGGAATTCGATGTAACTTATCAGAAGAAT | |
| | ATCTCAATACAAGATTCTCAAGTAATAATAACAGGCAATCTTGATATCTCAACT | |
| | GAGCTTGGGAATGTCAACAACTCGATCAGTAATGCTTTGAATAAGTTAGAGGA | |
| | AAGCAACAGAAAACTAGACAAAGTCAATGTCAAACTGACTAGCACATCTGCTC | |
| | TCATTACCTATATCGTTTTGACTATCATATCTCTTGTTTTTGGTATACTTAGCCTG | |
| | ATTCTAGCATGCTACCTAATGTACAAGCAAAAGGCGCAACAAAAGACCTTATT | |
| | ATGGCTTGGGAATAATACTCTAGATCAGATGAGAGCCACTACAAAAATGTGAA | |
| | CACAGATGAGGAACGAAGGTTTCCCTAATAGTAATTTGTGTGAAAGTTCTGGT | |
| | AGTCTGTCAGTTCAGAGAGTTAAGAAAAAACTACCGGTTGTAGATGACCAAAG | |
| | GACGATATACGGGTAGAACGGTAAGAGAGGCCGCCCCTCAATTGCGAGCCAG | |
| | GCTTCACAACCTCCGTTCTACCGCTTCACCGACAACAGTCCTCAATCATGGACCG | |
| | CGCCGTTAGCCAAGTTGCGTTAGAGAATGATGAAAGAGAGGCAAAAAATACA | |
| | TGGCGCTTGATATTCCGGATTGCAATCTTATTCTTAACAGTAGTGACCTTGGCT | |
| | ATATCTGTAGCCTCCCTTTTATATAGCATGGGGGCTAGCACACCTAGCGATCTT | |
| | GTAGGCATACCGACTAGGATTTCCAGGGCAGAAGAAAAGATTACATCTACACT | |
| | TGGTTCCAATCAAGATGTAGTAGATAGGATATATAAGCAAGTGGCCCTTGAGT | |
| | CTCCGTTGGCATTGTTAAATACTGAGACCACAATTATGAACGCAATAACATCTC | |
| | TCTCTTATCAGATTAATGGAGCTGCAAACAACAGTGGGTGGGGGGCACCTATC | |
| | CATGACCCAGATTATATAGGGGGGATAGGCAAAGAACTCATTGTAGATGATG | |
| | CTAGTGATGTCACATCATTCTATCCCTCTGCATTTCAAGAACATCTGAATTTTAT | |
| | CCCGGCGCCTACTACAGGATCAGGTTGCACTCGAATACCCTCATTTGACATGAG | |
| | TGCTACCCATTACTGCTACACCCATAATGTAATATTGTCTGGATGCAGAGATCA | |
| | CTCACATTCATATCAGTATTTAGCACTTGGTGTGCTCCGGACATCTGCAACAGG | |
| | GAGGGTATTCTTTTCTACTCTGCGTTCCATCAACCTGGACGACACCCAAAATCG | |
| | GAAGTCTTGCAGTGTGAGTGCAACTCCCCTGGGTTGTGATATGCTGTGCTCGA | |
| | AAGTCACGGAGACAGAGGAAGAAGATTATAACTCAGCTGTCCCTACGCGGAT | |
| | GGTACATGGGAGGTTAGGGTTCGACGGCCAGTACCACGAAAAGGACCTAGAT | |
| | GTCACAACATTATTCGGGGACTGGGTGGCCAACTACCCAGGAGTAGGGGGTG | |
| | GATCTTTTATTGACAGCCGCGTATGGTTCTCAGTCTACGGAGGGTTAAAACCCA | |
| | ATTCACCCAGTGACACTGTACAGGAAGGGAAATATGTGATATACAAGCGATAC | |
| | AATGACACATGCCCAGATGAGCAAGACTACCAGATTCGAATGGCCAAGTCTTC | |
| | GTATAAGCCTGGACGGTTTGGTGGGAAACGCATACAGCAGGCTATCTTATCTA | |
| | TCAAGGTGTCAACATCCTTAGGCGAAGACCCGGTACTGACTGTACCGCCCAAC | |
| | ACAGTCACACTCATGGGGGCCGAAGGCAGAATTCTCACAGTAGGGACATCTCA | |
| | TTTCTTGTATCAACGAGGGTCATCATACTTCTCTCCCGCGTTATTATATCCTATG | |
| | ACAGTCAGCAACAAAACAGCCACTCTTCATAGTCCTTATACATTCAATGCCTTC | |
| | ACTCGGCCAGGTAGTATCCCTTGCCAGGCTTCAGCAAGATGCCCCAACTCGTGT | |
| | GTTACTGGAGTCTATACAGATCCATATCCCCTAATCTTCTATAGAAACCACACCT | |
| | TGCGAGGGGTATTCGGGACAATGCTTGATGGTGTACAAGCAAGACTTAACCCT | |
| | GCGTCTGCAGTATTCGATAGCACATCCCGCAGTCGCATTACTCGAGTGAGTTCA | |
| | AGCAGTACCAAAGCAGCATACACAACATCAACTTGTTTTAAAGTGGTCAAGAC | |
| | TAATAAGACCTATTGTCTCAGCATTGCTGAAATATCTAATACTCTCTTCGGAGA | |
| | ATTCAGAATCGTCCCGTTACTAGTTGAGATCCTCAAAGATGACGGGGTTAGAG | |
| | AAGCCAGGTCTGGCTAGTTGAGTCAATTATAAAGGAGTTGGAAAGATGGCATT | |
| | GTATCACCTATCTTCTGCGACATCAAGAATCAAACCGAATGCCGGCGCGTGCTC | |
| | GAATTCCATGTTGCCAGTTGACCACAATCAGCCAGTGCTCATGCGATCAGATTA | |
| | AGCCTTGTCAATAGTCTCTTGATTAAGAAAAAATGTAAGTGGCAATGAGATAC | |
| | AAGGCAAAACAGCTCATGGTTAACAATACGGGTAGGACATGGCGAGCTCCGG | |
| | TCCTGAAAGGGCAGAGCATCAGATTATCCTACCAGAGTCACACCTGTCTTCACC | |
| | ATTGGTCAAGCACAAACTACTCTATTACTGGAAATTAACTGGGCTACCGCTTCC | |
| | TGATGAATGTGACTTCGACCACCTCATTCTCAGCCGACAATGGAAAAAAATACT | |
| | TGAATCGGCCTCTCCTGATACTGAGAGAATGATAAAACTCGGAAGGGCAGTAC | |
| | ACCAAACTCTTAACCACAATTCCAGAATAACCGGAGTGCTCCACCCCAGGTGTT | |
| | TAGAAGAACTGGCTAATATTGAGGTCCCAGATTCAACCAACAAATTTCGGAAG | |
| | ATTGAGAAGAAGATCCAAATTCACAACACGAGATATGGAGAACTGTTCACAA | |
| | GGCTGTGTACGCATATAGAGAAGAAACTGCTGGGGTCATCTTGGTCTAACAAT | |
| | GTCCCCCGGTCAGAGGAGTTCAGCAGCATTCGTACGGATCCGGCATTCTGGTTT | |
| | CACTCAAAATGGTCCACAGCCAAGTTTGCATGGCTCCATATAAAACAGATCCA | |
| | GAGGCATCTGATGGTGGCAGCTAGGACAAGGTCTGCGGCCAACAAATTGGTG | |
| | ATGCTAACCCATAAGGTAGGCCAAGTCTTTGTCACTCCTGAACTTGTCGTTGTG | |
| | ACGCATACGAATGAGAACAAGTTCACATGTCTTACCCAGGAACTTGTATTGAT | |
| | GTATGCAGATATGATGGAGGGCAGAGATATGGTCAACATAATATCAACCACG | |
| | GCGGTGCATCTCAGAAGCTTATCAGAGAAAATTGATGACATTTTGCGGTTAAT | |
| | AGACGCTCTGGCAAAAGACTTGGGTAATCAAGTCTACGATGTTGTATCACTAA | |
| | TGGAGGGATTTGCATACGGAGCTGTCCAGCTACTCGAGCCGTCAGGTACATTT | |
| | GCAGGAGATTTCTTCGCATTCAACCTGCAGGAGCTTAAAGACATTCTAATTGGC | |
| | CTCCTCCCCAATGATATAGCAGAATCCGTGACTCATGCAATCGCTACTGTATTCT | |
| | CTGGTTTAGAACAGAATCAAGCAGCTGAGATGTTGTGTCTGTTGCGTCTGTGG | |
| | GGTCACCCACTGCTTGAGTCCCGTATTGCAGCAAAGGCAGTCAGGAGCCAAAT | |
| | GTGCGCACCGAAAATGGTAGACTTTGATATGATCCTTCAGGTACTGTCTTTCTT | |
| | CAAGGGAACAATCATCAACGGGTACAGAAAGAAGAATGCAGGTGTGTGGCCG | |
| | CGAGTCAAAGTGGATACAATATATGGGAAGGTCATTGGGCAACTACATGCAG | |
| | ATTCAGCAGAGATTTCACACGATATCATGTTGAGAGAGTATAAGAGTTTATCT | |
| | GCACTTGAATTTGAGCCATGTATAGAATATGACCCTGTCACCAACCTGAGCATG | |
| | TTCCTAAAAGACAAGGCAATCGCACACCCCAACGATAATTGGCTTGCCTCGTTT | |
| | AGGCGGAACCTTCTCTCCGAAGACCAGAAGAAACATGTAAAAGAAGCAACTTC | |
| | GACTAATCGCCTCTTGATAGAGTTTTTAGAGTCAAATGATTTTGATCCATATAA | |
| | AGAGATGGAATATCTGACGACCCTTGAGTACCTTAGAGATGACAATGTGGCAG | |
| | TATCATACTCGCTCAAGGAGAAGGAAGTGAAAGTTAATGGACGGATCTTCGCT | |
| | AAGCTGACAAAGAAGTTAAGGAACTGTCAGGTGATGGCGGAAGGGATCCTAG | |
| | CCGATCAGATTGCACCTTTCTTTCAGGGAAATGGAGTCATTCAGGATAGCATAT | |
| | CCTTGACCAAGAGTATGCTAGCGATGAGTCAACTGTCTTTTAACAGCAATAAG | |
| | AAACGTATCACTGACTGTAAAGAAAGAGTATCTTCAAACCGCAATCATGATCC | |
| | GAAAAGCAAGAACCGTCGGAGAGTTGCAACCTTCATAACAACTGACCTGCAAA | |
| | AGTACTGTCTTAATTGGAGATATCAGACAATCAAATTGTTCGCTCATGCCATCA | |
| | ATCAGTTGATGGGCCTACCTCACTTCTTCGAATGGATTCACCTAAGACTGATGG | |
| | ACACTACGATGTTCGTAGGAGACCCTTTCAATCCTCCAAGTGACCCTACTGACT | |
| | GTGACCTCTCAAGAGTCCCTAATGATGACATATATATTGTCAGTGCCAGAGGG | |
| | GGTATCGAAGGATTATGCCAGAAGCTATGGACAATGATCTCAATTGCTGCAAT | |
| | CCAACTTGCTGCAGCTAGATCGCATTGTCGTGTTGCCTGTATGGTACAGGGTGA | |
| | TAATCAAGTAATAGCAGTAACGAGAGAGGTAAGATCAGACGACTCTCCGGAG | |
| | ATGGTGTTGACACAGTTGCATCAAGCCAGTGATAATTTCTTCAAGGAATTAATT | |
| | CATGTCAATCATTTGATTGGCCATAATTTGAAGGATCGTGAAACCATCAGGTCA | |
| | GACACATTCTTCATATACAGCAAACGAATCTTCAAAGATGGAGCAATCCTCAGT | |
| | CAAGTCCTCAAAAATTCATCTAAATTAGTGCTAGTGTCAGGTGATCTCAGTGAA | |
| | AACACCGTAATGTCCTGTGCCAACATTGCCTCTACTGTAGCACGGCTATGCGAG | |
| | AACGGGCTTCCCAAAGACTTCTGTTACTATTTAAACTATATAATGAGTTGTGTG | |
| | CAGACATACTTTGACTCTGAGTTCTCCATCACCAACAATTCGCACCCCGATCTTA | |
| | ATCAGTCGTGGATTGAGGACATCTCTTTTGTGCACTCATATGTTCTGACTCCTGC | |
| | CCAATTAGGGGGACTGAGTAACCTTCAATACTCAAGGCTCTACACTAGAAATA | |
| | TCGGTGACCCGGGGACTACTGCTTTTGCAGAGATCAAGCGACTAGAAGCAGTG | |
| | GGATTACTGAGTCCTAACATTATGACTAATATCTTAACTAGGCCGCCTGGGAAT | |
| | GGAGATTGGGCCAGTCTGTGCAACGACCCATACTCTTTCAATTTTGAGACTGTT | |
| | GCAAGCCCAAATATTGTTCTTAAGAAACATACGCAAAGAGTCCTATTTGAAACT | |
| | TGTTCAAATCCCTTATTGTCTGGAGTGCACACAGAGGATAATGAGGCAGAAGA | |
| | GAAGGCATTGGCTGAATTCTTGCTTAATCAAGAGGTGATTCATCCCCGCGTTGC | |
| | GCATGCCATCATGGAGGCAAGCTCTGTAGGTAGGAGAAAGCAAATTCAAGGG | |
| | CTTGTTGACACAACAAACACCGTAATTAAGATTGCGCTTACTAGGAGGCCATTA | |
| | GGCATCAAGAGGCTGATGCGGATAGTCAATTATTCTAGCATGCATGCAATGCT | |
| | GTTTAGAGACGATGTTTTTTCCTCCAGTAGATCCAACCACCCCTTAGTCTCTTCT | |
| | AATATGTGTTCTCTGACACTGGCAGACTATGCACGGAATAGAAGCTGGTCACC | |
| | TTTGACGGGAGGCAGGAAAATACTGGGTGTATCTAATCCTGATACGATAGAAC | |
| | TCGTAGAGGGTGAGATTCTTAGTGTAAGCGGAGGGTGTACAAGATGTGACAG | |
| | CGGAGATGAACAATTTACTTGGTTCCATCTTCCAAGCAATATAGAATTGACCGA | |
| | TGACACCAGCAAGAATCCTCCGATGAGGGTACCATATCTCGGGTCAAAGACAC | |
| | AGGAGAGGAGAGCTGCCTCACTTGCAAAAATAGCTCATATGTCGCCACATGTA | |
| | AAGGCTGCCCTAAGGGCATCATCCGTGTTGATCTGGGCTTATGGGGATAATGA | |
| | AGTAAATTGGACTGCTGCTCTTACGATTGCAAAATCTCGGTGTAATGTAAACTT | |
| | AGAGTATCTTCGGTTACTGTCCCCTTTACCCACGGCTGGGAATCTTCAACATAG | |
| | ACTAGATGATGGTATAACTCAGATGACATTCACCCCTGCATCTCTCTACAGGGT | |
| | GTCACCTTACATTCACATATCCAATGATTCTCAAAGGCTGTTCACTGAAGAAGG | |
| | AGTCAAAGAGGGGAATGTGGTTTACCAACAGATCATGCTCTTGGGTTTATCTCT | |
| | AATCGAATCGATCTTTCCAATGACAACAACCAGGACATATGATGAGATCACAC | |
| | TGCACCTACATAGTAAATTTAGTTGCTGTATCAGAGAAGCACCTGTTGCGGTTC | |
| | CTTTCGAGCTACTTGGGGTGGTACCGGAACTGAGGACAGTGACCTCAAATAAG | |
| | TTTATGTATGATCCTAGCCCTGTATCGGAGGGAGACTTTGCGAGACTTGACTTA | |
| | GCTATCTTCAAGAGTTATGAGCTTAATCTGGAGTCATATCCCACGATAGAGCTA | |
| | ATGAACATTCTTTCAATATCCAGCGGGAAGTTGATTGGCCAGTCTGTGGTTTCT | |
| | TATGATGAAGATACCTCCATAAAGAATGACGCCATAATAGTGTATGACAATAC | |
| | CCGAAATTGGATCAGTGAAGCTCAGAATTCAGATGTGGTCCGCCTATTTGAAT | |
| | ATGCAGCACTTGAAGTGCTCCTCGACTGTTCTTACCAACTCTATTACCTGAGAGT | |
| | AAGAGGCCTGGACAATATTGTCTTATATATGGGTGATTTATACAAGAATATGC | |
| | CAGGAATTCTACTTTCCAACATTGCAGCTACAATATCTCATCCCGTCATTCATTC | |
| | AAGGTTACATGCAGTGGGCCTGGTCAACCATGACGGATCACACCAACTTGCAG | |
| | ATACGGATTTTATCGAAATGTCTGCAAAACTATTAGTATCTTGCACCCGACGTG | |
| | TGATCTCCGGCTTATATTCAGGAAATAAGTATGATCTGCTGTTCCCATCTGTCTT | |
| | AGATGATAACCTGAATGAGAAGATGCTTCAGCTGATATCCCGGTTATGCTGTCT | |
| | GTACACGGTACTCTTTGCTACAACAAGAGAAATCCCGAAAATAAGAGGCTTAA | |
| | CTGCAGAAGAGAAATGTTCAATACTCACTGAGTATTTACTGTCGGATGCTGTG | |
| | AAACCATTACTTAGCCCCGATCAAGTGAGCTCTATCATGTCTCCTAACATAATTA | |
| | CATTCCCAGCTAATCTGTACTACATGTCTCGGAAGAGCCTCAATTTGATCAGGG | |
| | AAAGGGAGGACAGGGATACTATCCTGGCGTTGTTGTTCCCCCAAGAGCCATTA | |
| | TTAGAGTTCCCTTCTGTGCAAGATATTGGTGCTCGAGTGAAAGATCCATTCACC | |
| | CGACAACCTGCGGCATTTTTGCAAGAGTTAGATTTGAGTGCTCCAGCAAGGTA | |
| | TGACGCATTCACACTTAGTCAGATTCATCCTGAACTCACATCTCCAAATCCGGA | |
| | GGAAGACTACTTAGTACGATACTTGTTCAGAGGGATAGGGACTGCATCTTCCT | |
| | CTTGGTATAAGGCATCTCATCTCCTTTCTGTACCCGAGGTAAGATGTGCAAGAC | |
| | ACGGGAACTCCTTATACTTAGCTGAAGGGAGCGGAGCCATCATGAGTCTTCTC | |
| | GAACTGCATGTACCACATGAAACTATCTATTACAATACGCTCTTTTCAAATGAG | |
| | ATGAACCCCCCGCAACGACATTTCGGGCCGACCCCAACTCAGTTTTTGAATTCG | |
| | GTTGTTTATAGGAATCTACAGGCGGAGGTAACATGCAAAGATGGATTTGTCCA | |
| | AGAGTTCCGTCCATTATGGAGAGAAAATACAGAGGAAAGCGACCTGACCTCA | |
| | GATAAAGTAGTGGGGTATATTACATCTGCAGTGCCCTACAGATCTGTATCATTG | |
| | CTGCATTGTGACATTGAAATTCCTCCAGGGTCCAATCAAAGCTTACTAGATCAA | |
| | CTAGCTATCAATTTATCTCTGATTGCCATGCATTCTGTAAGGGAGGGCGGGGTA | |
| | GTAATCATCAAAGTGTTGTATGCAATGGGATACTACTTTCATCTACTCATGAAC | |
| | TTGTTTGCTCCGTGTTCCACAAAAGGATATATTCTCTCTAATGGTTATGCATGTC | |
| | GAGGAGATATGGAGTGTTACCTGGTATTTGTCATGGGTTACCTGGGCGGGCCT | |
| | ACATTTGTACATGAGGTGGTGAGGATGGCGAAAACTCTGGTGCAGCGGCACG | |
| | GTACGCTTTTGTCTAAATCAGATGAGATCACACTGACCAGGTTATTCACCTCAC | |
| | AGCGGCAGCGTGTGACAGACATCCTATCCAGTCCTTTACCAAGATTAATAAAG | |
| | TACTTGAGGAAGAATATTGACACTGCGCTGATTGAAGCCGGGGGACAGCCCGT | |
| | CCGTCCATTCTGTGCGGAGAGTCTGGTGAGCACGCTAGCGAACATAACTCAGA | |
| | TAACCCAGATCATCGCTAGTCACATTGACACAGTTATCCGGTCTGTGATATATA | |
| | TGGAAGCTGAGGGTGATCTCGCTGACACAGTATTTCTATTTACCCCTTACAATC | |
| | TCTCTACTGACGGGAAAAAGAGGACATCACTTAAACAGTGCACGAGACAGATC | |
| | CTAGAGGTTACAATACTAGGTCTTAGAGTCGAAAATCTCAATAAAATAGGCGA | |
| | TATAATCAGCCTAGTGCTTAAAGGCATGATCTCCATGGAGGACCTTATCCCACT | |
| | AAGGACATACTTGAAGCATAGTACCTGCCCTAAATATTTGAAGGCTGTCCTAG | |
| | GTATTACCAAACTCAAAGAAATGTTTACAGACACTTCTGTACTGTACTTGACTC | |
| | GTGCTCAACAAAAATTCTACATGAAAACTATAGGCAATGCAGTCAAAGGATAT | |
| | TACAGTAACTGTGACTCTTAACGAAAATCACATATTAATAGGCTCCTTTTTTGG | |
| | CCAATTGTATTCTTGTTGATTTAATCATATTATGTTAGAAAAAAGTTGAACCCTG | |
| | ACTCCTTAGGACTCGAATTCGAACTCAAATAAATGTCTTAAAAAAAGGTTGCGC | |
| | ACAATTATTCTTGAGTGTAGTCTCGTCATTCACCAAATCTTTGTTTGGTGCGCGC | |
| | GGCCGGCATGGTCCCAGCCTCCTCGCTGGCGCCGGCTGGGCAACATTCCGAGG | |
| | GGACCGTCCCCTCGGTAATGGCGAATGGGACGTCGACTGCTAACAAAGCCCGA | |
| | AAGGAAGCTGAGTTGGCTGCTGCCACCGCTGAGCAATAACTAGCATAACCCCTT | |
| | GGGGCCTCTAAACGGGTCTTGAGGGGTTTTTTGCTGAAAGGAGGAACTATATGC | |
| | GCGCAGATCTGTCATGATGATCATTGCAATTGGATCCATATATAGGGCCCGGGT | |
| | TATAATTACCTCAGGTCGACGTCCCATGGCCATTCGAATTCGTAATCATGGTCAT | |
| | AGCTGTTTCCTGTGTGAAATTGTTATCCGCTCACAATTCCACACAACATACGAGC | |
| | CGGAAGCATAAAGTGTAAAGCCTGGGGTGCCTAATGAGTGAGCTAACTCACATT | |
| | AATTGCGTTGCGCTCACTGCCCGCTTTCCAGTCGGGAAACCTGTCGTGCCAGCTG | |
| | CATTAATGAATCGGCCAACGCGCGGGGAGAGGCGGTTTGCGTATTGGGCGC | |
| Full length | ACGAAAAAGAAGAATAAAAGGCAGAAGCCTTTTAAAAGGAACCCTGGGCTGTC | 86 |
| genome of | GTAGGTGTGGGAAGGTTGTATTCCGAGTGCGCCTCCGAGGCATCTACTCTACA | |
| APMV4 | CCTATCACAATGGCTGGTGTCTTCTCCCAGTATGAGAGGTTTGTGGACAATCAA | |
| from RNA | TCCCAAGTGTCAAGGAAGGATCATCGGTCCTTAGCAGGAGGATGCCTTAAAGT | |
| sequencing | TAACATCCCTATGCTTGTCACTGCATCTGAAGACCCCACCACTCGTTGGCAACT | |
| and | AGCATGCTTATCTCTAAGGCTCCTGATCTCCAACTCATCAACCAGTGCTATCCG | |
| confirmation | TCAGGGGGCAATACTGACTCTCATGTCATTACCATCACAAAACATGAGAGCAAC | |
| of the 5’ and | AGCAGCTATTGCTGGTTCCACAAATGCAGCTGTTATCAACACCATGGAAGTCTT | |
| 3’ ends by | AAGTGTCAACGACTGGACCCCATCCTTCGACCCTAGGAGCGGTCTTTCTGAGG | |
| RACE | AAGATGCTCAAGTTTTCAGAGACATGGCAAGAGATCTGCCCCCTCAGTTCACCT | |
| | CTGGATCACCCTTCACATCAGCATTGGCGGAGGGGTTCACTCCTGAAGATACT | |
| | CATGACCTGATGGAGGCCTTGACCAGTGTGCTGATACAGATCTGGATCCTGGT | |
| | GGCTAAGGCCATGACCAACATTGACGGCTCTGGGGAGGCCAATGAAAGACGT | |
| | CTTGCAAAGTACATCCAAAAAGGACAGCTTAATCGTCAGTTTGCAATTGGTAAT | |
| | CCTGCCCGTCTGATAATCCAACAGACAATCAAAAGCTCCTTAACTGTCCGTAGG | |
| | TTCTTGGTCTCTGAGCTTCGTGCGTCACGAGGTGCAGTAAAAGAAGGATCCCC | |
| | TTACTATGCAGCTGTTGGGGATATCCACGCTTACATCTTTAATGCGGGATTGAC | |
| | ACCATTCTTGACCACCTTAAGATACGGGATAGGCACCAAGTACGCCGCTGTTG | |
| | CACTCAGTGTGTTCGCTGCAGATATTGCAAAGTTGAAGAGCCTACTTACCCTGT | |
| | ACCAGGACAAGGGTGTAGAAGCTGGATACATGGCACTCCTTGAGGATCCAGAC | |
| | TCCATGCACTTTGCACCTGGAAACTTCCCACACATGTACTCCTATGCAATGGGG | |
| | GTAGCTTCTTACCATGATCCTAGCATGCGCCAATACCAATACGCCAGGAGGTTC | |
| | CTCAGCCGTCCTTTCTACTTACTAGGAAGGGACATGGCCGCCAAGAACACAGG | |
| | CACGCTGGATGAGCAACTGGCGAAGGAACTGCAAGTATCAGAGAGAGATCGC | |
| | GCCGCATTATCCGCTGCGATTCAATCAGCGATGGAGGGGGGAGAGTCCGACG | |
| | ACTTCCCACTGTCGGGATCCATGCCGGCTCTCTCTGAGAATGCGCAACCAGTT | |
| | ACCCCCAGACCTCAACAGTCCCAGCTCTCTCCCCCCCAATCATCAAACATGCC | |
| | CCAATCAGCACCCAGGACCCCAGACTATCAACCCGACTTTGAACTGTAGGCTT | |
| | CATCACCGCACCAACAACAGCCCAAGAAGACCACCCCTCCCCCCACACATCTC | |
| | ACCCAGCCACCCATAAAGACTCAGTCCCACGCCCCAGCATCTCCTTCATTTAAT | |
| | TAAAAACCGACCAACAGGGTGGGGAAGGAGAGTCATTGGCTACTGCCAATTGT | |
| | GTGCAGCAATGGATTTTACTGACATTGATCTGTCAACTCATTGATCGAATCAT | |
| | CATCGGCAATCATAGACTCCATACAGCATGGAGGGCTGCAACCAGCGGGCAC | |
| | CGTCGGCCTATCGCAGATCCCAAAAGGGATAACCAGCGCATTAACCAAGGCCT | |
| | GGGAGGCTGAGGCGGCAACTGCCGGTAATGGGGACACCCAACACAAATCTGA | |
| | CAGTCCGGAGGATCATCAGGCCAACGACACAGATTCCCCTGAAGACACAGGTA | |
| | CTGACCAGACCACCCAGGAGGCCAACATCGTTGAGACACCCCACCCCGAGGT | |
| | GCTGTCAGCAGCCAAAGCCAGACTCAAGAGGCCCAAAGCAGGGAGGGACACC | |
| | CGCGACAACTCCCCTGCGCAACCCGATCATCTTTTAAGGGGGGCCTCCTGAG | |
| | CCCACAACCAGCAGCATCATGGGTGCAAAATCCACCCAGTCATGGAGGTCCCG | |
| | GCACCGCCGATCCCCGCCCATCACAAACTCAGGATCATTCCCCCACCGGAGA | |
| | GAAATGGCGATTGTCACCGACAAAGCAACCGGAGACATTGAACTGGTGGAGTG | |
| | GTGCAACCCGGGGTGCACAGCAGTCCGAATTGAACCCACCAGACTCGACTGT | |
| | GTATGCGGACACTGCCCCACCATCTGTAGCCTCTGCATGTATGACGACTGATC | |
| | AGGTACAACTACTAATGAAGGAGGTTGCTGACATAAAATCACTCCTTCAGGCGT | |
| | TAGTGAGGAACCTCGCTGTCTTGCCCCAATTGAGGAATGAGGTTGCAGCAATC | |
| | AGAACATCACAGGCCATGATAGAGGGGACACTCAATTCGATCAAGATTCTTGAC | |
| | CCTGGGAATTATCAGGAATCATCACTAAACAGTTGGTTCAAACCTCGCCAAGAT | |
| | CACACTGTTGTTGTGTCTGGACCAGGGAATCCATTGGCCATGCCAACCCCAGT | |
| | CCAAGACAACACCATATTCCTGGACGAGCTAGCCAGACCTCATCCTAGTGTGG | |
| | TCAATCCATCCCCGCCCATCACCAACACCAATGTTGACCTTGGCCCACAGAAG | |
| | CAGGCTGCAATAGCCTATATCTCCGCTAAATGCAAGGATCCGGGGAAACGAGA | |
| | TCAGCTATCAAGGCTCATTGAGCGAGCAACCACCCCAAGTGAGATCAACAAAG | |
| | TTAAAAGACAAGCCCTTGGGCTCTAGATCACTCGATCACCCCTCATGGTGATCA | |
| | CAACAATAATCAGAACCCTTCCGAACCACATGACCAACCCAGCCCACCGCCCA | |
| | CACCGTCCATCGACATCCCTTGCCAAACATCCTGCCGTAGCTGATTTATTCAAA | |
| | AGAGCTCATTTGATATGACCTGGTAATCATAAAATAGGGTGGGGAAGGTGCTTT | |
| | GCCTGTAAGGGGGCTCCCTCATCTTCAGACACGTGCCCGCCATCTCACCAACA | |
| | GTGCAATGGCAGACATGGACACGGTGTATATCAATCTGATGGCAGATGACCCA | |
| | ACCCACCAAAAAGAACTGCTGTCCTTTCCTCTCATCCCTGTGACCGGTCCTGAC | |
| | GGGAAGAAGGAACTCCAACACCAGATCCGGACCCAATCCTTGCTCGCCTCAGA | |
| | CAAACAAACTGAACGGTTCATCTTCCTCAACACTTACGGATTCATCTATGACAC | |
| | CACACCGGACAAGACAACTTTTTCCACCCCAGAGCATATTAATCAGCCTAAGAG | |
| | GACGACGGTGAGTGCCGCGATGATGACCATTGGCCTGGTTCCCGCCAATATAC | |
| | CCCTGAACGAACTAACGGCTACTGTGTTCAGCCTTAAAGTAAGAGTGAGGAAA | |
| | AGTGCGAGGTATCGGGAAGTGGTCTGGTATCAATGCAATCCAGTACCGGCCCT | |
| | GCTTGCAGCCACCAGGTTTGGTCGCCAAGGAGGTCTCGAGTCGAGCACTGGA | |
| | GTCAGTGTAAAGGCTCCCGAGAAGATAGATTGTGAGAAGGATTATACCTACTAC | |
| | CCTTATTTCTTATCTGTGTGCTACATCGCCACCTCCAACCTGTTCAAGGTACCG | |
| | AGGATGGTTGCTAATGCAACCAACAGTCAATTATACCACCTTACCATGCAGGTC | |
| | ACATTTGCCTTTCCAAAAAACATCCCTCCAGCCAACCAGAAACTCCTGACACAG | |
| | GTGGATGAGGGATTCGAGGGCACTGTGGATTGCCATTTTGGGAACATGCTGAA | |
| | AAAGGATCGGAAAGGGAACATGAGGACACTGTCCCAGGCGGCAGATAAGGTC | |
| | AGACGAATGAATATTCTTGTTGGTATCTTTGACTTGCATGGGCCAACGCTCTTC | |
| | CTGGAGTATACCGGGAAACTGACAAAGGCTCTGCTAGGGTTCATGTCCACCAG | |
| | CCGAACAGCAATCATCCCCATATCTCAGCTCAATCCCATGCTGAGTCAACTCAT | |
| | GTGGAGCAGTGATGCCCAGATAGTAAAGTTAAGGGTTGTCATAACTACATCCAA | |
| | ACGCGGCCCGTGCGGGGGTGAGCAGGAGTATGTGCTGGATCCCAAATTCACA | |
| | GTTAAGAAAGAAAAGGCTCGACTCAACCCTTTCAAGAAGGCAGCCTAATGATTT | |
| | AATCCGCAAGATCCCAGAAATCAGACCACTCTATACTATCCACTGATCACTGGA | |
| | AATGTAATTGTACAGTTGATGAATCTGTGAAGAATCAATTAAAAAACCGGATCCT | |
| | TATTAGGGTGGGGAAGTAGTTGATTGGGTGTCTAAACAAAAGCATTTCTTCACA | |
| | CCTCCCCGCCACGAAACAACCACAATGAGGCTATCAAACACAATCTTGACCTTG | |
| | ATTCTCATCATACTTACCGGCTATTTGATAGGTGTCCACTCCACCGATGTGAAT | |
| | GAGAAACCAAAGTCCGAAGGGATTAGGGGTGATCTTACACCAGGTGCGGGTAT | |
| | TTTCGTAACTCAAGTCCGACAGCTCCAGATCTACCAACAGTCTGGGTACCATGA | |
| | TCTTGTCATCAGATTGTTACCTCTTCTACCAACAGAGCTTAATGATTGTCAAAGG | |
| | GAAGTTGTCACAGAGTACAATAACACTGTATCACAGCTGTTGCAGCCTATCAAA | |
| | ACCAACCTGGATACTTTGTTGGCAGATGGTAGCACAAGGGATGTTGATATACAG | |
| | CCGCGATTCATTGGGGCAATAATAGCCACAGGTGCCCTGGCTGTAGCAACGGT | |
| | AGCTGAGGTAACTGCAGCTCAAGCACTATCTCAGTCAAAAACGAATGCTCAAAA | |
| | TATTCTCAAGTTGAGAGATAGTATTCAGGCCACCAACCAAGCAGTTTTTGAAATT | |
| | TCACAGGGACTCGAAGCAACTGCAACCGTGCTATCAAAACTGCAAACTGAGCT | |
| | CAATGAGAATATCATCCCAAGTCTGAACAACTTGTCCTGTGCTGCCATGGGGAA | |
| | TCGCCTTGGTGTATCACTCTCACTCTATTTGACCTTAATGACCACTCTATTTGGG | |
| | GACCAGATCACAAACCCAGTGCTGACGCCAATCTCTTACAGCACCCTATCGGC | |
| | AATGGCGGGTGGTCACATTGGTCCAGTGATGAGTAAGATATTAGCCGGATCTG | |
| | TCACAAGTCAGTTGGGGGCAGAACAACTGATTGCTAGTGGCTTAATACAGTCA | |
| | CAGGTAGTAGGTTATGATTCCCAGTATCAGCTGTTGGTTATCAGGGTCAACCTT | |
| | GTACGGATTCAGGAAGTCCAGAATACTAGGGTTGTATCACTAAGAACACTAGCA | |
| | GTCAATAGGGATGGTGGACTTTACAGAGCCCAGGTGCCACCCGAGGTAGTTGA | |
| | GCGATCTGGCATTGCAGAGCGGTTTTATGCAGATGATTGTGTTCTAACTACAAC | |
| | TGATTACATCTGCTCATCGATCCGATCTTCTCGGCTTAATCCAGAGTTAGTCAA | |
| | GTGTCTCAGTGGGGCACTTGATTCATGCACATTTGAGAGGGAAAGTGCATTACT | |
| | GTCAACTCCCTTCTTTGTATACAACAAGGCAGTCGTCGCAAATTGTAAAGCAGC | |
| | GACATGTAGATGTAATAAACCGCCATCTATCATTGCCCAATACTCTGCATCAGC | |
| | TCTAGTAACCATCACCACCGACACTTGTGCTGACCTTGAAATTGAGGGTTATCG | |
| | TTTCAACATACAGACTGAATCCAACTCATGGGTTGCACCAAACTTCACGGTCTC | |
| | AACCTCACAAATAGTATCGGTTGATCCAATAGACATATCCTCTGACATTGCCAA | |
| | AATTAACAATTCTATCGAGGCTGCGCGAGAGCAGCTGGAACTGAGCAACCAGA | |
| | TCCTTTCCCGAATCAACCCACGGATTGTGAACGACGAATCACTAATAGCTATTA | |
| | TCGTGACAATTGTTGTGCTTAGTCTCCTTGTAATTGGTCTTATTATTGTTCTCGG | |
| | TGTGATGTACAAGAATCTTAAGAAAGTCCAACGAGCTCAAGCTGCTATGATGAT | |
| | GCAGCAAATGAGCTCATCACAGCCTGTGACCACCAAATTGGGGACACCCTTCT | |
| | AGGTGAATAATCATATCAATCCATTCAATAATGAGCGGGACATACCAATCACCA | |
| | ACGACTGTGTCACAAGGCCGGTTAGGAATGCACCGGATCTCTCTCCTTCCTTTT | |
| | TAATTAAAAACGGTTGAACTGAGGGTGAGGGGGGGGGTGTGCATGGTAGGGT | |
| | GGGGAAGGTAGCCAATTCCTGCCCATTGGGCCGACCGTACCAAGAGAAGTCA | |
| | ACAGAAGTATAGATGCAGGGCGACATGGAGGGTAGCCGTGATAACCTCACAGT | |
| | AGATGATGAATTAAAGACAACATGGAGGTTAGCTTATAGAGTTGTATCCCTCCT | |
| | ATTGATGGTGAGTGCCTTGATAATCTCTATAGTAATCCTGACGAGAGATAACAG | |
| | CCAAAGCATAATCACGGCGATCAACCAGTCGTATGACGCAGACTCAAAGTGGC | |
| | AAACAGGGATAGAAGGGAAAATCACCTCAATCATGACTGATACGCTCGATACCA | |
| | GGAATGCAGCTCTTCTCCACATTCCACTCCAGCTCAATACACTTGAGGCAAACC | |
| | TGTTGTCCGCCCTCGGAGGTAACACGGGAATTGGCCCCGGAGATCTAGAGCA | |
| | CTGTCGTTATCCGGTTCATGACTCCGCTTACCTGCATGGAGTCAATCGATTACT | |
| | CATCAATCAAACAGCTGACTACACAGCAGAAGGCCCCCTGGATCATGTGAACT | |
| | TCATTCCGGCACCAGTTACGACTACTGGATGCACAAGGATCCCATCCTTTTCTG | |
| | TATCATCATCCATTTGGTGCTATACACACAATGTGATTGAAACAGGTTGCAATGA | |
| | CCACTCAGGTAGTAATCAATATATCAGTATGGGGGTGATTAAGAGGGCTGGCA | |
| | ACGGCTTACCTTACTTCTCAACAGTCGTGAGTAAGTATCTGACCGATGGGTTGA | |
| | ATAGAAAAAGCTGTTCCGTAGCTGCCGGATCCGGGCATTGTTACCTCCTTTGTA | |
| | GCCTAGTGTCAGAGCCCGAACCTGATGACTATGTGTCACCAGATCCCACACCG | |
| | ATGAGGTTAGGGGTGCTAACAAGGGATGGGTCTTACACTGAACAGGTGGTACC | |
| | CGAAAGAATATTTAAGAACATATGGAGCGCAAACTACCCTGGGGTAGGGTCAG | |
| | GTGCTATAGTAGGAAATAAGGTGTTATTCCCATTTTACGGCGGAGTGAAGAATG | |
| | GATCAACCCCTGAGGTGATGAATAGGGGAAGATATTACTACATCCAGGATCCA | |
| | AATGACTATTGCCCTGACCCGCTGCAAGATCAGATCTTAAGGGCAGAACAATC | |
| | GTATTATCCTACTCGATTTGGTAGGAGGATGGTAATGCAGGGAGTCCTAACATG | |
| | TCCAGTATCCAACAATTCAACAATAGCCAGCCAATGCCAATCTTACTATTTCAAC | |
| | AACTCATTAGGATTCATCGGGGCGGAATCTAGGATCTATTACCTCAATGGTAAC | |
| | ATTTACCTTTATCAAAGAAGCTCGAGCTGGTGGCCTCACCCCCAAATTTACCTA | |
| | CTTGATTCCAGGATTGCAAGTCCGGGTACGCAGAACATTGACTCAGGCGTTAA | |
| | CCTCAAGATGTTAAATGTTACTGTCATTACACGACCATCATCTGGCTTTTGTAAT | |
| | AGTCAGTCAAGATGCCCTAATGACTGCTTATTCGGGGTTTATTCAGATGTCTGG | |
| | CCTCTTAGCCTTACCTCAGACAGCATATTTGCATTTACAATGTACTTACAAGGGA | |
| | AGACGACACGTATTGACCCAGCTTGGGCGCTATTCTCCAATCATGTAATTGGG | |
| | CATGAGGCTCGTTTGTTCAACAAGGAGGTTAGTGCTGCTTATTCTACCACCACT | |
| | TGTTTTTCGGACACCATCCAAAACCAGGTGTATTGTCTGAGTATACTTGAAGTC | |
| | AGAAGTGAGCTCTTGGGGGCATTCAAGATAGTGCCATTCCTCTATCGTGTCTTA | |
| | TAGGCACCTGCTTGGTCAAGAACCCTGAGCAGCCATAAAATTAACACTTGATCT | |
| | TCCTTAAAAACACCTATCTAAATTACTGTCTGAGATCCCTGATTAGTTACCCTTT | |
| | CAATCAATCAATTAATTTTTAATTAAAAACGGAAAAATGGGCCTAGTTCCAAGGA | |
| | AAGGATGGGACCCATTAGGGTGGGGAAGGATTACTTTGTTCCTTGACTCGCAC | |
| | CCACGTACACCCAATCCCATTCCTGTCAAGAAGGAACCCTTCCCAAACTCACCT | |
| | TGCAATGTCCAATCAGGCAGCTGAGATTATACTACCCACCTTCCATCTGGAATC | |
| | ACCCTTGATCGAGAATAAGTGCTTCTACTACATGCAATTACTTGGTCTCGTGTTA | |
| | CCACATGATCACTGGAGATGGAGGGCATTCGTCAA1111ACAGTGGATCAAGC | |
| | ACACCTTAAAAATCGTAATCCCCGCTTAATGGCCCACATCGATCACACTAAGGA | |
| | TAGACTAAGGGCTCATGGTGTCTTGGGTTTCCACCAGACTCAGACAAGTGAGA | |
| | GCCGTTTCCGTGTCTTGCTCCATCCTGAAACTTTACCTTGGCTATCAGCAATGG | |
| | GAGGATGCATCAACCAGGTTCCCAAGGCATGGCGGAACACTCTGAAATCTATC | |
| | GAGCACAGTGTGAAGCAGGAGGCGACTCAACTGAAGTTACTCATGGAAAAAAC | |
| | CTCACTAAAGCTAACAGGAGTATCTTACTTATTCTCCAATTGCAATCCCGGGAA | |
| | AACTGCAGCGGGAACTATGCCCGTACTAAGTGAGATGGCATCAGAACTCTTGT | |
| | CAAATCCCATCTCCCAATTCCAATCAACATGGGGGTGTGCTGCTTCAGGGTGG | |
| | CACCATGTAGTCAGCATCATGAGGCTCCAACAGTATCAAAGAAGGACAGGTAA | |
| | GGAAGAGAAAGCAATCACTGAAGTTCAGTATGGCTCGGACACCTGTCTCATTAA | |
| | TGCAGACTACACCGTCGTTTTTTCCTCACAGGACCGTGTCATAGCAGTCTTGC | |
| | CTTTCGATGTTGTCCTCATGATGCAAGACCTGCTTGAATCCCGACGGAATGTCT | |
| | TGTTCTGTGCCCGCTTTATGTATCCCAGAAGCCAACTACATGAGAGGATAAGTA | |
| | CAATACTGGCCCTTGGAGACCAACTCGGGAGAAAAGCACCCCAAGTCCTGTAT | |
| | GATTTCGTAGCTACCCTCGAATCATTTGCATACGCTGCTGTCCAACTTCATGAC | |
| | AACAACCCTATCTACGGTGGGGCTTTCTTTGAGTTCAATATCCAAGAACTGGAA | |
| | GCTA1111GTCCCCTGCACTTAATAAGGATCAAGTCAACTTCTACATAAGTCAAG | |
| | TTGTCTCAGCATACAGTAACCTTCCCCCATCTGAATCAGCAGAATTGCTATGCT | |
| | TACTACGCCTGTGGGGTCATCCCTTGCTAAACAGTCTTGATGCAGCAAAGAAA | |
| | GTCAGAGAATCTATGTGTGCTGGGAAGGTTCTTGATTATAATGCTATTCGACTA | |
| | GIIIIGICIIIIIATCATACGTTATTAATCAATGGGTATCGGAAGAAACATAAGG | |
| | GTCGCTGGCCAAATGTGAATCAACATTCACTACTCAACCCGATAGTGAAGCAG | |
| | CTTTACTTTGATCAGGAGGAGATCCCACACTCTGTTGCCCTTGAGCACTATTTA | |
| | GATATCTCGATGATAGAATTTGAGAAGACTTTTGAAGTGGAACTATCTGATAGT | |
| | CTAAGCATCTTTCTGAAGGATAAGTCGATAGCTTTGGATAAACAAGAATGGCAC | |
| | AGTGGTTTTGTCTCAGAAGTGACTCCAAAGCACCTACGAATGTCTCGTCATGAT | |
| | CGCAAGTCTACCAATAGGCTATTGTTAGCCTTTATTAACTCCCCTGAATTCGAT | |
| | GTTAAGGAAGAGCTTAAATATTTGACTACAGGTGAGTATGCCACTGACCCAAAT | |
| | TTCAATGTCTCTTACTCACTGAAAGAGAAGGAAGTTAAGAAAGAAGGGCGCATT | |
| | TTCGCAAAGATGTCACAGAAAATGAGAGCATGCCAGGTTATTTGTGAAGAGTTA | |
| | CTAGCACATCATGTGGCTCCTTTGTTTAAAGAGAATGGTGTTACACAATCGGAG | |
| | CTATCCCTGACAAAGAATTTGTTGGCTATTAGCCAACTGAGTTACAACTCGATG | |
| | GCCGCTAAGGTGCGATTGCTGAGGCCAGGGGACAAGTTCACCGCTGCACACT | |
| | ATATGACCACAGACCTAAAAAAGTACTGCCTTAACTGGCGGCACCAGTCAGTCA | |
| | AATTGTTCGCCAGAAGCCTGGATCGACTATTTGGGTTAGACCATGCTTTTTCTT | |
| | GGATACACGTCCGTCTCACCAATAGCACTATGTACGTTGCTGACCCATTCAATC | |
| | CACCAGACTCAGATGCATGCACAAATTTAGACGACAATAAGAACACTGGGATTT | |
| | TTATTATAAGTGCTCGAGGTGGTATAGAAGGCCTTCAACAGAAACTATGGACTG | |
| | GCATATCAATTGCAATCGCCCAGGCGGCAGCAGCCCTCGAGGGCTTACGAATT | |
| | GCTGCCACTTTGCAGGGGGATAACCAGGTTTTAGCGATTACGAAAGAATTCAT | |
| | GACCCCAGTCTCGGAGGATGTAATCCACGAGCAGCTATCTGAAGCGATGTCGC | |
| | GATACAAGAGGACTTTCACATACCTTAATTATTTAATGGGGCACCAATTGAAGG | |
| | ATAAAGAAACCATCCAATCCAGTGACTTCTTCGTTTACTCCAAAAGGATCTTCTT | |
| | CAATGGGTCAATCCTAAGTCAATGCCTCAAGAACTTCAGTAAACTCACTACCAA | |
| | TGCCACTACCCTTGCTGAGAACACTGTAGCCGGCTGCAGTGACATCTCCTCAT | |
| | GCATAGCCCGTTGTGTGGAAAACGGGTTGCCTAAGGATGCTGCATATGTTCAG | |
| | AATATAATCATGACTCGGCTTCAACTGTTGCTAGATCACTACTATTCTATGCATG | |
| | GTGGCATAAACTCAGAGTTAGAGCAGCCAACTCTAAGTATCCCTGTCCGAAAC | |
| | GCAACCTATTTACCATCTCAATTAGGCGGTTACAATCATTTGAATATGACCCGA | |
| | CTATTCTGTCGCAATATCGGTGACCCGCTTACTAGTTCTTGGGCAGAGTCAAAA | |
| | AGACTAATGGATGTTGGCCTTCTCAGTCGTAAGTTCTTAGAGGGGATATTATGG | |
| | AGACCCCGGGAAGTGGGACATTTTCAACACTCATGCTTGATCCGTTCGCACT | |
| | TAACATTGATTACTTAAGGCCACCAGAGACAATAATCCGAAAACACACCCAAAA | |
| | AGTCTTGTTGCAGGATTGTCCTAATCCTCTATTAGCAGGTGTAGTTGACCCGAA | |
| | CTACAACCAGGAATTAGAATTATTAGCTCAGTTCCTGCTTGATCGGGAAACCGT | |
| | TATTCCCAGGGCTGCCCATGCCATCTTTGAACTGTCTGTCTTGGGAAGGAAAAA | |
| | ACATATACAAGGATTGGTTGATACTACAAAAACAATTATTCAGTGCTCATTAGAA | |
| | AGACAGCCACTGTCCTGGAGGAAAGTTGAGAACATTGTAACCTACAATGCGCA | |
| | GTATTTCCTCGGGGCCACCCAGCAGGTTGACACCAATATCTCAGAAAGGCAGT | |
| | GGGTGATGCCAGGTAATTTCAAGAAGCTTGTATCTCTTGACGATTGCTCAGTCA | |
| | CGTTGTCCACTGTGTCACGGCGCATTTCTTGGGCCAATCTACTTAACTGGAGG | |
| | GCTATAGATGGTTTGGAAACTCCAGATGTGATAGAGAGTATTGATGGCCGCCTT | |
| | GTGCAATCATCCAATCAATGCGGCCTATGTAATCAAGGATTGGGCTCCTACTCC | |
| | TGGTTCTTCTTGCCCTCCGGGTGTGTGTTCGACCGTCCACAAGATTCTCGAGT | |
| | GGTTCCAAAGATGCCATACGTGGGATCCAAAACGGATGAGAGACAGACTGCGT | |
| | CAGTGCAGGCTATACAGGGATCCACATGTCACCTTAGAGCAGCATTGAGACTT | |
| | GTATCACTCTACCTTTGGGCCTATGGAGATTCTGACATATCATGGCTAGAAGCC | |
| | GCGACATTGGCTCAAACACGGTGCAATATTTCTCTTGATGACCTGCGGATCCTG | |
| | AGCCCTCTTCCTTCCTCGGCAAATTTACACCACAGATTGAATGACGGGGTAACA | |
| | CAAGTGAAATTCATGCCCGCCACATCGAGCCGGGTGTCAAAGTTCGTCCAAAT | |
| | TTGCAATGACAACCAGAATCTTATCCGTGATGATGGGAGTGTTGATTCCAATAT | |
| | GATTTATCAGCAGGTTATGATATTAGGGCTTGGAGAGATTGAATGTTTGTTAGC | |
| | TGACCCAATCGATACAAACCCAGAACAACTGATTCTTCACCTACACTCTGATAA | |
| | TTCTTGCTGTCTCCGGGAGATGCCAACGACCGGTTTTGTACCTGCTTTAGGATT | |
| | GACCCCATGCTTAACTGTCCCAAAGCACAATCCGTATATTTATGATGATAGCCC | |
| | AATACCCGGTGATTTGGATCAGAGGCTCATTCAAACCAAATTCTTTATGGGTTC | |
| | TGACAATCTAGATAATCTTGATATCTACCAGCAGCGAGCTTTACTGAGTCGGTG | |
| | TGTGGCTTATGACATTATCCAATCAGTATTCGCTTGCGATGCACCAGTATCTCA | |
| | GAAGAATGATGCAATCCTTCACACTGACTACCATGAAAATTGGATCTCAGAGTT | |
| | CCGATGGGGTGACCCTCGCATAATCCAAGTAACAGCAGGTTACGAGTTAATTC | |
| | TGTTCCTTGCATACCAGCTTTATTATCTCAGAGTGAGGGGTGACCGTGCAATCC | |
| | TGTGTTATATTGATAGGATACTCAACAGGATGGTATCTTCCAATCTAGGCAGTC | |
| | TCATCCAGACGCTCTCTCATCCGGAGATTAGGAGGAGATTTTCATTGAGTGATC | |
| | AAGGGTTCCTTGTCGAAAGGGAGCTAGAGCCAGGTAAGCCACTGGTAAAACAA | |
| | GCGGTTATGTTCCTAAGGGACTCAGTCCGCTGCGCTTTAGCAACTATCAAGGC | |
| | AGGAATTGAGCCTGAGATCTCCCGAGGTGGCTGTACCCAGGATGAGCTGAGCT | |
| | TTACCCTTAAGCACTTACTATGTCGGCGTCTCTGTATAATTGCTCTCATGCATTC | |
| | GGAAGCAAAGAACTTGGTCAAAGTTAGAAACCTTCCAGTAGAGGAAAAAACCG | |
| | CCTTACTATACCAGATGTTGATCACTGAGGCCAATGCCAGGAGATCAGGGTCT | |
| | GCTAGTATCATCATAAGCTTAGTTTCAGCACCCCAGTGGGACATTCATACACCA | |
| | GCGTTGTATTTTGTATCAAAGAAAATGCTGGGGATGCTCAAAAGGTCAACCACA | |
| | CCCTTGGATATAAGTGACCTTTCTGAGAGCCAGAACCTCACACCAACAGAATTG | |
| | AATGATGTTCCTGGTCACATGGCAGAGGAATTTCCCTGTTTGTTTAGCAGTTAT | |
| | AACGCTACATATGAAGACACAATTACTTACAATCCAATGACTGAAAAACTCGCA | |
| | GTGCACTTGGACAATGGTTCCACCCCTTCCAGAGCGCTTGGTCGTCACTACAT | |
| | CCTGCGACCCCTTGGGCTTTACTCGTCTGCATGGTACCGGTCTGCAGCACTAT | |
| | TAGCGTCAGGGGCCCTCAGTGGGTTGCCTGAGGGGTCAAGCCTGTACTTGGG | |
| | AGAGGGGTATGGGACCACCATGACTCTACTTGAGCCCGTTGTCAAGTCCTCAA | |
| | CTGTTTACTACCATACATTGTTTGACCCAACCCGGAATCCTTCACAGCGGAACT | |
| | ACAAACCAGAACCGCGGGTATTCACTGATTCCATTTGGTACAAGGATGATTTCA | |
| | CACGACCACCTGGTGGCATTGTAAATCTATGGGGTGAAGACGTACGTCAGAGT | |
| | GATATTACACAGAAAGACACGGTTAATTTCATATTATCTCGGGTCCCGCCAAAA | |
| | TCACTCAAATTGATACACGTTGATATTGAGTTCTCCCCAGACTCTGATGTACGG | |
| | ACGCTACTATCTGGCTATTCCCATTGTGCACTATTGGCCTACTGGCTACTGCAA | |
| | CCTGGAGGGCGATTTGCGGTTAGAGTTTTCTTAAGTGACCATATCATAGTCAAC | |
| | TTGGTCACTGCCATTCTGTCCGCTTTTGACTCTAATCTGGTGTGCATTGCGTCA | |
| | GGATTGACACACAAGGATGATGGGGCAGGTTATATTTGTGCAAAGAAGCTTGC | |
| | AAATGTTGAGGCTTCAAGAATTGAGTATTACTTGAGGATGGTCCACGGCTGTGT | |
| | TGACTCATTAAAAATTCCTCATCAATTAGGAATCATTAAATGGGCTGAGGGTGA | |
| | AGTGTCCCGACTTACCAAAAAGGCGGATGATGAAATAAACTGGCGGTTAGGTG | |
| | ATCCAGTTACCAGATCATTTGATCCGGTTTCTGAGCTAATAATTGCGCGAACAG | |
| | GGGGATCAGTATTAATGGAATACGGGACTTTTACTAACCTCAGGTGTGCGAACT | |
| | TGGGCAGATACATATAAACTTTTGGCTTCAATTGTAGAGACCACCTTAATGGAAAT | |
| | AAGGGTTGAGCAAGATCAGTTGGAAGATGATTCGAGGAGACAAATCCAGGTAG | |
| | TCCCTGCTTTTAATACAAGATCCGGGGAAGGATCCGTACATTGATTGAGTGTG | |
| | CTCAGCTGCAGGTCATAGATGTTATCTGTGTGAACATAGATCACCTCTTTCCCA | |
| | AACACCGACATGCTCTTGTCACACAACTTACTTACCAGTCAGTGTGCCTTGGGG | |
| | ACTTGATTGAAGGCCCCCAAATTAAGACATATCTAAGGGCCAGGAAGTGGATC | |
| | CAACGTAGGGGACTCAATGAGACAATTAACCATATCATCACTGGACAAGTGTCG | |
| | CGGAATAAGGCAAGGGATTTTTTCAAGAGGCGCCTGAAGTTGGTTGGCTTTTC | |
| | GCTCTGTGGCGGTTGGGGCTACCTCTCACTTTAGCTGCTTAGATTGTTGATTAT | |
| | TATGAATAATCGGAGTCGAAATCGTAAATAGAAAGACATAAAATTGCAAATAAG | |
| | CAATGATCGTATTAATATTTAATAAAAAATATGTCTTTTATTTCTT | |
| Additional | ccgcggTTAGAAAAAATACGGGTAGAACCGCCACCATGCATCTGCTGTGTTTCCT | 87 |
| transcription | GTCGCTCGCCTGCTCACTGCTGGCGGCGGCACTTATCCCGTCCCCACGGGAG | |
| unit | GCTCCTGCCACCGTGGCCGCCTTCGAATCTGGGCTGGGCTTCAGCGAAGCCG | |
| encoding | AGCCCGATGGCGGAGAGGTCAAGGCATTCGAAGGAAAGGACCTCGAAGAAC | |
| mVEGF-C. | AGCTGAGATCCGTGTCCTCCGTGGACGAACTCATGTCCGTCCTGTACCCCGA | |
| (size = 1290 | TTACTGGAAGATGTACAAATGCCAGCTCCGGAAGGGCGGTTGGCAGCAGCC | |
| nt, rule of | CACTCTGAACACTCGCACGGGAGATTCCGTGAAGTTTGCCGCCGCCCACTAC | |
| 6). | AATACTGAGATTCTCAAGTCCATCGACAACGAATGGAGGAAAACCCAGTGTA | |
| Open reading | TGCCGCGCGAAGTCTGCATTGACGTGGGAAAGGAGTTCGGCGCTGCCACCA | |
| frame in | ACACCTTCTTTAAGCCTCCCTGCGTGTCGGTGTATCGCTGCGGGGGATGCTG | |
| bold. | CAACAGCGAAGGCCTTCAGTGCATGAACACCAGCACCGGATACCTCAGCAA | |
| | GACTCTCTTCGAAATCACTGTGCCGCTGTCACAAGGCCCGAAGCCTGTGACC | |
| | ATTTCCTTCGCCAACCACACCTCCTGTCGGTGCATGAGCAAGCTGGATGTGTA | |
| | CAGACAGGTGCACTCCATCATCCGGAGATCGTTGCCTGCCACCCTGCCGCAG | |
| | TGCCAAGCGGCTAACAAGACCTGTCCCACCAACTACGTGTGGAACAACTATA | |
| | TGTGTCGGTGCCTGGCACAGCAGGACTTTATCTTCTACTCCAACGTGGAGGA | |
| | CGACTCGACTAACGGTTTCCACGACGTGTGCGGACCCAACAAGGAGCTGGAT | |
| | GAGGATACTTGTCAGTGCGTGTGCAAGGGTGGCCTGCGCCCGTCCTCCTGCG | |
| | GACCACATAAGGAACTGGACAGGGACTCGTGCCAATGCGTCTGCAAGAACA | |
| | AGCTGTTCCCTAACTCCTGCGGGGCGAACCGCGAATTCGACGAGAACACCTG | |
| | TCAGTGTGTGTGCAAGCGGACTTGCCCGAGGAATCAGCCTCTTAACCCCGGA | |
| | AAATGCGCCTGCGAATGCACAGAGAACACCCAGAAGTGCTTCTTGAAAGGG | |
| | AAGAAGTTCCACCACCAAACCTGTTCATGCTACCGGCGCCCATGTGCCAACC | |
| | GGCTGAAGCACTGCGACCCGGGATTGAGCTTCAGCGAGGAGGTCTGCAGAT | |
| | GCGTGCCGTCATACTGGAAGCGACCTCATCTGAATTAGCccgcgg | |
| Rescue | TCTTCCGCTTCCTCGCTCACTGACTCGCTGCGCTCGGTCGTTCGGCTGCGGCGAG | 88 |
| plasmid | CGGTATCAGCTCACTCAAAGGCGGTAATACGGTTATCCACAGAATCAGGGGATA | |
| pNDV- | ACGCAGGAAAGAACATGTGAGCAAAAGGCCAGCAAAAGGCCAGGAACCGTAA | |
| mVEGF-C | AAAAATCGACGCTCAAGTCAGAGGTGGCGAAACCCGACAGGACTATAAAGATA | |
| with the | AAAAATCGACGCTCAAGTCAGAGGTGGCGAAACCCGACAGGACTATAAAGATA | |
| mVEGF-C | CCAGGCGTTTCCCCCTGGAAGCTCCCTCGTGCGCTCTCCTGTTCCGACCCTGCCG | |
| transcription | CTTACCGGATACCTGTCCGCCTTTCTCCCTTCGGGAAGCGTGGCGCTTTCTCATA | |
| unit inserted | GCTCACGCTGTAGGTATCTCAGTTCGGTGTAGGTCGTTCGCTCCAAGCTGGGCT | |
| at the unique | GTGTGCACGAACCCCCCGTTCAGCCCGACCGCTGCGCCTTATCCGGTAACTATCG | |
| Sac II site, | TCTTGAGTCCAACCCGGTAAGACACGACTTATCGCCACTGGCAGCAGCCACTGG | |
| (viral | TAACAGGATTAGCAGAGCGAGGTATGTAGGCGGTGCTACAGAGTTCTTGAAGT | |
| sequences in | GGTGGCCTAACTACGGCTACACTAGAAGAACAGTATTTGGTATCTGCGCTCTGCT | |
| bold). CTA | GAAGCCAGTTACCTTCGGAAAAAGAGTTGGTAGCTCTTGATCCGGCAAACAAAC | |
| = Leu | CACCGCTGGTAGCGGTGGTTTTTTTGTTTGCAAGCAGCAGATTACGCGCAGAAA | |
| changed to | AAAAGGATCTCAAGAAGATCCTTTGATCTTTTCTACGGGGTCTGACGCTCAGTGG | |
| GCC = Ala | AACGAAAACTCACGTTAAGGGATTTTGGTCATGAGATTATCAAAAAGGATCTTC | |
| (underlined). | ACCTAGATCCTTTTAAATTAAAAATGAAGTTTTAAATCAATCTAAAGTATATATGA | |
| | GTAAACTTGGTCTGACAGTTACCAATGCTTAATCAGTGAGGCACCTATCTCAGCG | |
| | ATCTGTCTATTTCGTTCATCCATAGTTGCCTGACTCCCCGTCGTGTAGATAACTAC | |
| | GATACGGGAGGGCTTACCATCTGGCCCCAGTGCTGCAATGATACCGCGAGACCC | |
| | ACGCTCACCGGCTCCAGATTTATCAGCAATAAACCAGCCAGCCGGAAGGGCCGA | |
| | GCGCAGAAGTGGTCCTGCAACTTTATCCGCCTCCATCCAGTCTATTAATTGTTGC | |
| | CGGGAAGCTAGAGTAAGTAGTTCGCCAGTTAATAGTTTGCGCAACGTTGTTGCC | |
| | ATTGCTACAGGCATCGTGGTGTCACGCTCGTCGTTTGGTATGGCTTCATTCAGCT | |
| | CCGGTTCCCAACGATCAAGGCGAGTTACATGATCCCCCATGTTGTGCAAAAAAG | |
| | CGGTTAGCTCCTTCGGTCCTCCGATCGTTGTCAGAAGTAAGTTGGCCGCAGTGTT | |
| | ATCACTCATGGTTATGGCAGCACTGCATAATTCTCTTACTGTCATGCCATCCGTAA | |
| | GATGCTTTTCTGTGACTGGTGAGTACTCAACCAAGTCATTCTGAGAATAGTGTAT | |
| | GCGGCGACCGAGTTGCTCTTGCCCGGCGTCAATACGGGATAATACCGCGCCACA | |
| | TAGCAGAACTTTAAAAGTGCTCATCATTGGAAAACGTTCTTCGGGGCGAAAACT | |
| | CTCAAGGATCTTACCGCTGTTGAGATCCAGTTCGATGTAACCCACTCGTGCACCC | |
| | AACTGATCTTCAGCATCTTTTACTTTCACCAGCGTTTCTGGGTGAGCAAAAACAG | |
| | GAAGGCAAAATGCCGCAAAAAAGGGAATAAGGGCGACACGGAAATGTTGAAT | |
| | ACTCATACTCTTCCTTTTTCAATATTATTGAAGCATTTATCAGGGTTATTGTCTCAT | |
| | GAGCGGATACATATTTGAATGTATTTAGAAAAATAAACAAATAGGGGTTCCGCG | |
| | CACATTTCCCCGAAAAGTGCCACCTGACGTCTAAGAAACCATTATTATCATGACA | |
| | TTAACCTATAAAAATAGGCGTATCACGAGGCCCTTTCGTCTCGCGCGTTTCGGTG | |
| | ATGACGGTGAAAACCTCTGACACATGCAGCTCCCGGAGACGGTCACAGCTTGTC | |
| | TGTAAGCGGATGCCGGGAGCAGACAAGCCCGTCAGGGCGCGTCAGCGGGTGTT | |
| | GGCGGGTGTCGGGGCTGGCTTAACTATGCGGCATCAGAGCAGATTGTACTGAG | |
| | AGTGCACCATAAAATTGTAAACGTTAATATTTTGTTAAAATTCGCGTTAATTTTT | |
| | GTTAAATCAGCTCATTTTTTAACCAATAGGCCGAAATCGGCAAAATCCCTTATAA | |
| | ATCAAAAGAATAGCCCGAGATAGGGTTGAGTGTTGTTCCAGTTTGGAACAAGAG | |
| | TCCACTATTAAAGAACGTGGACTCCAACGTCAAAGGGCGAAAAACCGTCTATCA | |
| | GGGCGATGGCCCACTACGTGAACCATCACCCAAATCAAGTTTTTTGGGGTCGAG | |
| | GTGCCGTAAAGCACTAAATCGGAACCCTAAAGGGAGCCCCCGATTTAGAGCTTG | |
| | ACGGGGAAAGCCGGCGAACGTGGCGAGAAAGGAAGGGAAGAAAGCGAAAGG | |
| | AGCGGGCGCTAGGGCGCTGGCAAGTGTAGCGGTCACGCTGCGCGTAACCACCA | |
| | CACCCGCCGCGCTTAATGCGCCGCTACAGGGCGCGTACTATGGTTGCTTTGACG | |
| | TATGCGGTGTGAAATACCGCACAGATGCGTAAGGAGAAAATACCGCATCAGGC | |
| | GCCATTCGCCATTCAGGCTGCGCAACTGTTGGGAAGGGCGATCGGTGCGGGCCT | |
| | CTTCGCTATTACGCCAGCTGGCGAAAGGGGGATGTGCTGCAAGGCGATTAAGTT | |
| | GGGTAACGCCAGGGTTTTCCCAGTCACGACGTTGTAAAACGACGGCCAGTGCCA | |
| | AGCTTTAATACGACTCACTATAGGGACCAAACAGAGAATCCGTGAGTTACGATA | |
| | AAAGGCGAAGGAGCAATTGAAGTCGCACGGGTAGAAGGTGTGAATCTCGAGT | |
| | GCGAGCCCGAAGCACAAACTCGAGAAAGCCTTCTGCCAACATGTCTTCCGTATT | |
| | TGATGAGTACGAACAGCTCCTCGCGGCTCAGACTCGCCCCAATGGAGCTCATG | |
| | GAGGGGGAGAAAAAGGGAGTACCTTAAAAGTAGACGTCCCGGTATTCACTCT | |
| | TAACAGTGATGACCCAGAAGATAGATGGAGCTTTGTGGTATTCTGCCTCCGGA | |
| | TTGCTGTTAGCGAAGATGCCAACAAACCACTCAGGCAAGGTGCTCTCATATCTC | |
| | TTTTATGCTCCCACTCACAGGTAATGAGGAACCATGTTGCCCTTGCAGGGAAAC | |
| | AGAATGAAGCCACATTGGCCGTGCTTGAGATTGATGGCTTTGCCAACGGCACG | |
| | CCCCAGTTCAACAATAGGAGTGGAGTGTCTGAAGAGAGAGCACAGAGATTTG | |
| | CGATGATAGCAGGATCTCTCCCTCGGGCATGCAGCAACGGAACCCCGTTCGTC | |
| | ACAGCCGGGGCCGAAGATGATGCACCAGAAGACATCACCGATACCCTGGAGA | |
| | GGATCCTCTCTATCCAGGCTCAAGTATGGGTCACAGTAGCAAAAGCCATGACT | |
| | GCGTATGAGACTGCAGATGAGTCGGAAACAAGGCGAATCAATAAGTATATGC | |
| | AGCAAGGCAGGGTCCAAAAGAAATACATCCTCTACCCCGTATGCAGGAGCACA | |
| | ATCCAACTCACGATCAGACAGTCTCTTGCAGTCCGCATCTTTTTGGTTAGCGAG | |
| | CTCAAGAGAGGCCGCAACACGGCAGGTGGTACCTCTACTTATTATAACCTGGT | |
| | AGGGGACGTAGACTCATACATCAGGAATACCGGGCTTACTGCATTCTTCTTGAC | |
| | ACTCAAGTACGGAATCAACACCAAGACATCAGCCCTTGCACTTAGTAGCCTCTC | |
| | AGGCGACATCCAGAAGATGAAGCAGCTCATGCGTTTGTATCGGATGAAAGGA | |
| | GATAATGCGCCGTACATGACATTACTTGGTGATAGTGACCAGATGAGCTTTGC | |
| | GCCTGCCGAGTATGCACAACTTTACTCCTTTGCCATGGGTATGGCATCAGTCCT | |
| | AGATAAAGGTACTGGGAAATACCAATTTGCCAGGGACTTTATGAGCACATCAT | |
| | TCTGGAGACTTGGAGTAGAGTACGCTCAGGCTCAGGGAAGTAGCATTAACGA | |
| | GGATATGGCTGCCGAGCTAAAGCTAACCCCAGCAGCAAGGAGGGGCCTGGCA | |
| | GCTGCTGCCCAACGGGTCTCCGAGGAGACCAGCAGCATAGACATGCCTACTCA | |
| | ACAAGTCGGAGTCCTCACTGGGCTTAGCGAGGGGGGGTCCCAAGCTCTACAAG | |
| | GCGGATCGAATAGATCGCAAGGGCAACCAGAAGCCGGGGATGGGGAGACCC | |
| | AATTCCTGGATCTGATGAGAGCGGTAGCAAATAGCATGAGGGAGGCGCCAAA | |
| | CTCTGCACAGGGCACTCCCCAATCGGGGCCTCCCCCAACTCCTGGGCCATCCCA | |
| | AGATAACGACACCGACTGGGGGTATTGATGGACAAAACCCAGCCTGCTTCCAC | |
| | AAAAACATCCCAATGCCCTCACCCGTAGTCGACCCCTCGATTTGCGGCTCTATAT | |
| | GACCACACCCTCAAACAAACATCCCCCTCTTTCCTCCCTCCCCCTGCTGTACAACT | |
| | ACGTACGCCCTAGATACCACAGGCACAATGCGGCTCACTAACAATCAAAACAG | |
| | AGCCGAGGGAATTAGAAAAAAGTACGGGTAGAAGAGGGATATTCAGAGATC | |
| | AGGGCAAGTCTCCCGAGTCTCTGCTCTCTCCTCTACCTGATAGACCAGGACAAA | |
| | CATGGCCACCTTTACAGATGCAGAGATCGACGAGCTATTTGAGACAAGTGGAA | |
| | CTGTCATTGACAACATAATTACAGCCCAGGGTAAACCAGCAGAGACTGTTGGA | |
| | AGGAGTGCAATCCCACAAGGCAAGACCAAGGTGCTGAGCGCAGCATGGGAGA | |
| | AGCATGGGAGCATCCAGCCACCGGCCAGTCAAGACAACCCCGATCGACAGGA | |
| | CAGATCTGACAAACAACCATCCACACCCGAGCAAACGACCCCGCATGACAGCC | |
| | CGCCGGCCACATCCGCCGACCAGCCCCCCACCCAGGCCACAGACGAAGCCGTC | |
| | GACACACAGCTCAGGACCGGAGCAAGCAACTCTCTGCTGTTGATGCTTGACAA | |
| | GCTCAGCAATAAATCGTCCAATGCTAAAAAGGGCCCATGGTCGAGCCCCCAAG | |
| | AGGGGAATCACCAACGTCCGACTCAACAGCAGGGGAGTCAACCCAGTCGCGG | |
| | AAACAGTCAGGAAAGACCGCAGAACCAAGTCAAGGCCGCCCCTGGAAACCAG | |
| | GGCACAGACGTGAACACAGCATATCATGGACAATGGGAGGAGTCACAACTAT | |
| | CAGCTGGTGCAACCCCTCATGCTCTCCGATCAAGGCAGAGCCAAGACAATACC | |
| | CTTGTATCTGCGGATCATGTCCAGCCACCTGTAGACTTTGTGCAAGCGATGATG | |
| | TCTATGATGGAGGCGATATCACAGAGAGTAAGTAAGGTTGACTATCAGCTAGA | |
| | TCTTGTCTTGAAACAGACATCCTCCATCCCTATGATGCGGTCCGAAATCCAACA | |
| | GCTGAAAACATCTGTTGCAGTCATGGAAGCCAACTTGGGAATGATGAAGATTC | |
| | TGGATCCCGGTTGTGCCAACATTTCATCTCTGAGTGATCTACGGGCAGTTGCCC | |
| | GATCTCACCCGGTTTTAGTTTCAGGCCCTGGAGACCCCTCTCCCTATGTGACACA | |
| | AGGAGGCGAAATGGCACTTAATAAACTTTCGCAACCAGTGCCACATCCATCTG | |
| | AATTGATTAAACCCGCCACTGCATGCGGGCCTGATATAGGAGTGGAAAAGGA | |
| | CACTGTCCGTGCATTGATCATGTCACGCCCAATGCACCCGAGTTCTTCAGCCAA | |
| | GCTCCTAAGCAAGTTAGATGCAGCCGGGTCGATCGAGGAAATCAGGAAAATC | |
| | AAGCGCCTTGCTCTAAATGGCTAATTACTACTGCCACACGTAGCGGGTCCCTGT | |
| | CCACTCGGCATCACACGGAATCTGCACCGAGTTCCCCCCCGCGGTTAGAAAAAA | |
| | TACGGGTAGAACCGCCACCATGCATCTGCTGTGTTTCCTGTCGCTCGCCTGCTCA | |
| | CTGCTGGCGGCGGCACTTATCCCGTCCCCACGGGAGGCTCCTGCCACCGTGGCC | |
| | GCCTTCGAATCTGGGCTGGGCTTCAGCGAAGCCGAGCCCGATGGCGGAGAGGT | |
| | CAAGGCATTCGAAGGAAAGGACCTCGAAGAACAGCTGAGATCCGTGTCCTCCGT | |
| | GGACGAACTCATGTCCGTCCTGTACCCCGATTACTGGAAGATGTACAAATGCCA | |
| | GCTCCGGAAGGGCGGTTGGCAGCAGCCCACTCTGAACACTCGCACGGGAGATT | |
| | CCGTGAAGTTTGCCGCCGCCCACTACAATACTGAGATTCTCAAGTCCATCGACAA | |
| | CGAATGGAGGAAAACCCAGTGTATGCCGCGCGAAGTCTGCATTGACGTGGGAA | |
| | AGGAGTTCGGCGCTGCCACCAACACCTTCTTTAAGCCTCCCTGCGTGTCGGTGTA | |
| | TCGCTGCGGGGGATGCTGCAACAGCGAAGGCCTTCAGTGCATGAACACCAGCA | |
| | CCGGATACCTCAGCAAGACTCTCTTCGAAATCACTGTGCCGCTGTCACAAGGCCC | |
| | GAAGCCTGTGACCATTTCCTTCGCCAACCACACCTCCTGTCGGTGCATGAGCAAG | |
| | CTGGATGTGTACAGACAGGTGCACTCCATCATCCGGAGATCGTTGCCTGCCACC | |
| | CTGCCGCAGTGCCAAGCGGCTAACAAGACCTGTCCCACCAACTACGTGTGGAAC | |
| | AACTATATGTGTCGGTGCCTGGCACAGCAGGACTTTATCTTCTACTCCAACGTGG | |
| | AGGACGACTCGACTAACGGTTTCCACGACGTGTGCGGACCCAACAAGGAGCTG | |
| | GATGAGGATACTTGTCAGTGCGTGTGCAAGGGTGGCCTGCGCCCGTCCTCCTGC | |
| | GGACCACATAAGGAACTGGACAGGGACTCGTGCCAATGCGTCTGCAAGAACAA | |
| | GCTGTTCCCTAACTCCTGCGGGGCGAACCGCGAATTCGACGAGAACACCTGTCA | |
| | GTGTGTGTGCAAGCGGACTTGCCCGAGGAATCAGCCTCTTAACCCCGGAAAATG | |
| | CGCCTGCGAATGCACAGAGAACACCCAGAAGTGCTTCTTGAAAGGGAAGAAGT | |
| | TCCACCACCAAACCTGTTCATGCTACCGGCGCCCATGTGCCAACCGGCTGAAGCA | |
| | CTGCGACCCGGGATTGAGCTTCAGCGAGGAGGTCTGCAGATGCGTGCCGTCATA | |
| | CTGGAAGCGACCTCATCTGAATTAGCccgcggACCCAAGGTCCAACTCTCCAAGC | |
| | GGCAATCCTCTCTCGCTTCCTCAGCCCCACTGAATGATCGCGTAACCGTAATTAA | |
| | TCTAGCTACATTTAAGATTAAGAAAAAATACGGGTAGAATTGGAGTGCCCCAA | |
| | TTGTGCCAAGATGGACTCATCTAGGACAATTGGGCTGTACTTTGATTCTGCCCA | |
| | TTCTTCTAGCAACCTGTTAGCATTTCCGATCGTCCTACAAGACACAGGAGATGG | |
| | GAAGAAGCAAATCGCCCCGCAATATAGGATCCAGCGCCTTGACTTGTGGACTG | |
| | ATAGTAAGGAGGACTCAGTATTCATCACCACCTATGGATTCATCTTTCAAGTTG | |
| | GGAATGAAGAAGCCACCGTCGGCATGATCGATGATAAACCCAAGCGCGAGTT | |
| | ACTTTCCGCTGCGATGCTCTGCCTAGGAAGCGTCCCAAATACCGGAGACCTTAT | |
| | TGAGCTGGCAAGGGCCTGTCTCACTATGATAGTCACATGCAAGAAGAGTGCAA | |
| | CTAATACTGAGAGAATGGTTTTCTCAGTAGTGCAGGCACCCCAAGTGCTGCAA | |
| | AGCTGTAGGGTTGTGGCAAACAAATACTCATCAGTGAATGCAGTCAAGCACGT | |
| | GAAAGCGCCAGAGAAGATTCCCGGGAGTGGAACCCTAGAATACAAGGTGAAC | |
| | TTTGTCTCCTTGACTGTGGTACCGAAGAGGGATGTCTACAAGATCCCAGCTGCA | |
| | GTATTGAAGGTTTCTGGCTCGAGTCTGTACAATCTTGCGCTCAATGTCACTATT | |
| | AATGTGGAGGTAGACCCGAGGAGTCCTTTGGTTAAATCTCTGTCTAAGTCTGA | |
| | CAGCGGATACTATGCTAACCTCTTCTTGCATATTGGACTTATGACCACTGTAGA | |
| | TAGGAAGGGGAAGAAAGTGACATTTGACAAGCTGGAAAAGAAAATAAGGAG | |
| | CCTTGATCTATCTGTCGGGCTCAGTGATGTGCTCGGGCCTTCCGTGTTGGTAAA | |
| | AGCAAGAGGTGCACGGACTAAGCTTTTGGCACCTTTCTTCTCTAGCAGTGGGA | |
| | CAGCCTGCTATCCCATAGCAAATGCTTCTCCTCAGGTGGCCAAGATACTCTGGA | |
| | GTCAAACCGCGTGCCTGCGGAGCGTTAAAATCATTATCCAAGCAGGTACCCAA | |
| | CGCGCTGTCGCAGTGACCGCCGACCACGAGGTTACCTCTACTAAGCTGGAGAA | |
| | GGGGCACACCCTTGCCAAATACAATCCTTTTAAGAAATAAGCTGCGTCTCTGAG | |
| | ATTGCGCTCCGCCCACTCACCCAGATCATCATGACACAAAAAACTAATCTGTCTT | |
| | GATTATTTACAGTTAGTTTACCTGTCTATCAAGTTAGAAAAAACACGGGTAGAA | |
| | GATTCTGGATCCCGGTTGGCGCCCTCCAGGTGCAAGATGGGCTCCAGACCTTCT | |
| | ACCAAGAACCCAGCACCTATGATGCTGACTATCCGGGTTGCGCTGGTACTGAG | |
| | TTGCATCTGTCCGGCAAACTCCATTGATGGCAGGCCTCTTGCAGCTGCAGGAAT | |
| | TGTGGTTACAGGAGACAAAGCCGTCAACATATACACCTCATCCCAGACAGGAT | |
| | CAATCATAGTTAAGCTCCTCCCGAATCTGCCCAAGGATAAGGAGGCATGTGCG | |
| | AAAGCCCCCTTGGATGCATACAACAGGACATTGACCACTTTGCTCACCCCCCTT | |
| | GGTGACTCTATCCGTAGGATACAAGAGTCTGTGACTACATCTGGAGGGGGGA | |
| | GACAGGGGCGCCTTATAGGCGCCATTATTGGCGGTGTGGCTCTTGGGGTTGCA | |
| | ACTGCCGCACAAATAACAGCGGCCGCAGCTCTGATACAAGCCAAACAAAATGC | |
| | TGCCAACATCCTCCGACTTAAAGAGAGCATTGCCGCAACCAATGAGGCTGTGC | |
| | ATGAGGTCACTGACGGATTATCGCAACTAGCAGTGGCAGTTGGGAAGATGCA | |
| | GCAGTTTGTTAATGACCAATTTAATAAAACAGCTCAGGAATTAGACTGCATCA | |
| | AAATTGCACAGCAAGTTGGTGTAGAGCTCAACCTGTACCTAACCGAATTGACT | |
| | ACAGTATTCGGACCACAAATCACTTCACCTGCTTTAAACAAGCTGACTATTCAG | |
| | GCACTTTACAATCTAGCTGGTGGAAATATGGATTACTTATTGACTAAGTTAGGT | |
| | GTAGGGAACAATCAACTCAGCTCATTAATCGGTAGCGGCTTAATCACCGGTAA | |
| | CCCTATTCTATACGACTCACAGACTCAACTCTTGGGTATACAGGTAACT 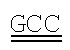 CCT | |
| | TCAGTCGGGAACCTAAATAATATGCGTGCCACCTACTTGGAAACCTTATCCGTA | |
| | AGCACAACCAGGGGATTTGCCTCGGCACTTGTCCCAAAAGTGGTGACACAGGT | |
| | CGGTTCTGTGATAGAAGAACTTGACACCTCATACTGTATAGAAACTGACTTAG | |
| | ATTTATATTGTACAAGAATAGTAACGTTCCCTATGTCCCCTGGTATTTATTCCTG | |
| | CTTGAGCGGCAATACGTCGGCCTGTATGTACTCAAAGACCGAAGGCGCACTTA | |
| | CTACACCATACATGACTATCAAAGGTTCAGTCATCGCCAACTGCAAGATGACAA | |
| | CATGTAGATGTGTAAACCCCCCGGGTATCATATCGCAAAACTATGGAGAAGCC | |
| | GTGTCTCTAATAGATAAACAATCATGCAATGTTTTATCCTTAGGCGGGATAACT | |
| | TTAAGGCTCAGTGGGGAATTCGATGTAACTTATCAGAAGAATATCTCAATACA | |
| | AGATTCTCAAGTAATAATAACAGGCAATCTTGATATCTCAACTGAGCTTGGGA | |
| | ATGTCAACAACTCGATCAGTAATGCTTTGAATAAGTTAGAGGAAAGCAACAGA | |
| | AAACTAGACAAAGTCAATGTCAAACTGACTAGCACATCTGCTCTCATTACCTAT | |
| | ATCGTTTTGACTATCATATCTCTTGTTTTTGGTATACTTAGCCTGATTCTAGCATG | |
| | CTACCTAATGTACAAGCAAAAGGCGCAACAAAAGACCTTATTATGGCTTGGGA | |
| | ATAATACTCTAGATCAGATGAGAGCCACTACAAAAATGTGAACACAGATGAG | |
| | GAACGAAGGTTTCCCTAATAGTAATTTGTGTGAAAGTTCTGGTAGTCTGTCAGT | |
| | TCAGAGAGTTAAGAAAAAACTACCGGTTGTAGATGACCAAAGGACGATATAC | |
| | GGGTAGAACGGTAAGAGAGGCCGCCCCTCAATTGCGAGCCAGGCTTCACAACC | |
| | TCCGTTCTACCGCTTCACCGACAACAGTCCTCAATCATGGACCGCGCCGTTAGC | |
| | CAAGTTGCGTTAGAGAATGATGAAAGAGAGGCAAAAAATACATGGCGCTTGA | |
| | TATTCCGGATTGCAATCTTATTCTTAACAGTAGTGACCTTGGCTATATCTGTAGC | |
| | CTCCCTTTTATATAGCATGGGGGCTAGCACACCTAGCGATCTTGTAGGCATACC | |
| | GACTAGGATTTCCAGGGCAGAAGAAAAGATTACATCTACACTTGGTTCCAATC | |
| | AAGATGTAGTAGATAGGATATATAAGCAAGTGGCCCTTGAGTCTCCGTTGGCA | |
| | TTGTTAAATACTGAGACCACAATTATGAACGCAATAACATCTCTCTCTTATCAG | |
| | ATTAATGGAGCTGCAAACAACAGTGGGTGGGGGGCACCTATCCATGACCCAG | |
| | ATTATATAGGGGGGATAGGCAAAGAACTCATTGTAGATGATGCTAGTGATGTC | |
| | ACATCATTCTATCCCTCTGCATTTCAAGAACATCTGAATTTTATCCCGGCGCCTA | |
| | CTACAGGATCAGGTTGCACTCGAATACCCTCATTTGACATGAGTGCTACCCATT | |
| | ACTGCTACACCCATAATGTAATATTGTCTGGATGCAGAGATCACTCACATTCAT | |
| | ATCAGTATTTAGCACTTGGTGTGCTCCGGACATCTGCAACAGGGAGGGTATTCT | |
| | TTTCTACTCTGCGTTCCATCAACCTGGACGACACCCAAAATCGGAAGTCTTGCA | |
| | GTGTGAGTGCAACTCCCCTGGGTTGTGATATGCTGTGCTCGAAAGTCACGGAG | |
| | ACAGAGGAAGAAGATTATAACTCAGCTGTCCCTACGCGGATGGTACATGGGA | |
| | GGTTAGGGTTCGACGGCCAGTACCACGAAAAGGACCTAGATGTCACAACATTA | |
| | TTCGGGGACTGGGTGGCCAACTACCCAGGAGTAGGGGGTGGATCTTTTATTGA | |
| | CAGCCGCGTATGGTTCTCAGTCTACGGAGGGTTAAAACCCAATTCACCCAGTG | |
| | ACACTGTACAGGAAGGGAAATATGTGATATACAAGCGATACAATGACACATG | |
| | CCCAGATGAGCAAGACTACCAGATTCGAATGGCCAAGTCTTCGTATAAGCCTG | |
| | GACGGTTTGGTGGGAAACGCATACAGCAGGCTATCTTATCTATCAAGGTGTCA | |
| | ACATCCTTAGGCGAAGACCCGGTACTGACTGTACCGCCCAACACAGTCACACTC | |
| | ATGGGGGCCGAAGGCAGAATTCTCACAGTAGGGACATCTCATTTCTTGTATCA | |
| | ACGAGGGTCATCATACTTCTCTCCCGCGTTATTATATCCTATGACAGTCAGCAAC | |
| | AAAACAGCCACTCTTCATAGTCCTTATACATTCAATGCCTTCACTCGGCCAGGTA | |
| | GTATCCCTTGCCAGGCTTCAGCAAGATGCCCCAACTCGTGTGTTACTGGAGTCT | |
| | ATACAGATCCATATCCCCTAATCTTCTATAGAAACCACACCTTGCGAGGGGTAT | |
| | TCGGGACAATGCTTGATGGTGTACAAGCAAGACTTAACCCTGCGTCTGCAGTA | |
| | TTCGATAGCACATCCCGCAGTCGCATTACTCGAGTGAGTTCAAGCAGTACCAAA | |
| | GCAGCATACACAACATCAACTTGTTTTAAAGTGGTCAAGACTAATAAGACCTAT | |
| | TGTCTCAGCATTGCTGAAATATCTAATACTCTCTTCGGAGAATTCAGAATCGTCC | |
| | CGTTACTAGTTGAGATCCTCAAAGATGACGGGGTTAGAGAAGCCAGGTCTGGC | |
| | TAGTTGAGTCAATTATAAAGGAGTTGGAAAGATGGCATTGTATCACCTATCTTC | |
| | TGCGACATCAAGAATCAAACCGAATGCCGGCGCGTGCTCGAATTCCATGTTGC | |
| | CAGTTGACCACAATCAGCCAGTGCTCATGCGATCAGATTAAGCCTTGTCAATAG | |
| | TCTCTTGATTAAGAAAAAATGTAAGTGGCAATGAGATACAAGGCAAAACAGCT | |
| | CATGGTTAACAATACGGGTAGGACATGGCGAGCTCCGGTCCTGAAAGGGCAG | |
| | AGCATCAGATTATCCTACCAGAGTCACACCTGTCTTCACCATTGGTCAAGCACA | |
| | AACTACTCTATTACTGGAAATTAACTGGGCTACCGCTTCCTGATGAATGTGACT | |
| | TCGACCACCTCATTCTCAGCCGACAATGGAAAAAAATACTTGAATCGGCCTCTC | |
| | CTGATACTGAGAGAATGATAAAACTCGGAAGGGCAGTACACCAAACTCTTAAC | |
| | CACAATTCCAGAATAACCGGAGTGCTCCACCCCAGGTGTTTAGAAGAACTGGC | |
| | TAATATTGAGGTCCCAGATTCAACCAACAAATTTCGGAAGATTGAGAAGAAGA | |
| | TCCAAATTCACAACACGAGATATGGAGAACTGTTCACAAGGCTGTGTACGCAT | |
| | ATAGAGAAGAAACTGCTGGGGTCATCTTGGTCTAACAATGTCCCCCGGTCAGA | |
| | GGAGTTCAGCAGCATTCGTACGGATCCGGCATTCTGGTTTCACTCAAAATGGTC | |
| | CACAGCCAAGTTTGCATGGCTCCATATAAAACAGATCCAGAGGCATCTGATGG | |
| | TGGCAGCTAGGACAAGGTCTGCGGCCAACAAATTGGTGATGCTAACCCATAAG | |
| | GTAGGCCAAGTCTTTGTCACTCCTGAACTTGTCGTTGTGACGCATACGAATGAG | |
| | AACAAGTTCACATGTCTTACCCAGGAACTTGTATTGATGTATGCAGATATGATG | |
| | GAGGGCAGAGATATGGTCAACATAATATCAACCACGGCGGTGCATCTCAGAA | |
| | GCTTATCAGAGAAAATTGATGACATTTTGCGGTTAATAGACGCTCTGGCAAAA | |
| | GACTTGGGTAATCAAGTCTACGATGTTGTATCACTAATGGAGGGATTTGCATA | |
| | CGGAGCTGTCCAGCTACTCGAGCCGTCAGGTACATTTGCAGGAGATTTCTTCGC | |
| | ATTCAACCTGCAGGAGCTTAAAGACATTCTAATTGGCCTCCTCCCCAATGATAT | |
| | AGCAGAATCCGTGACTCATGCAATCGCTACTGTATTCTCTGGTTTAGAACAGAA | |
| | TCAAGCAGCTGAGATGTTGTGTCTGTTGCGTCTGTGGGGTCACCCACTGCTTGA | |
| | GTCCCGTATTGCAGCAAAGGCAGTCAGGAGCCAAATGTGCGCACCGAAAATG | |
| | GTAGACTTTGATATGATCCTTCAGGTACTGTCTTTCTTCAAGGGAACAATCATC | |
| | AACGGGTACAGAAAGAAGAATGCAGGTGTGTGGCCGCGAGTCAAAGTGGAT | |
| | ACAATATATGGGAAGGTCATTGGGCAACTACATGCAGATTCAGCAGAGATTTC | |
| | ACACGATATCATGTTGAGAGAGTATAAGAGTTTATCTGCACTTGAATTTGAGCC | |
| | ATGTATAGAATATGACCCTGTCACCAACCTGAGCATGTTCCTAAAAGACAAGG | |
| | CAATCGCACACCCCAACGATAATTGGCTTGCCTCGTTTAGGCGGAACCTTCTCT | |
| | CCGAAGACCAGAAGAAACATGTAAAAGAAGCAACTTCGACTAATCGCCTCTTG | |
| | ATAGAGTTTTTAGAGTCAAATGATTTTGATCCATATAAAGAGATGGAATATCT | |
| | GACGACCCTTGAGTACCTTAGAGATGACAATGTGGCAGTATCATACTCGCTCA | |
| | AGGAGAAGGAAGTGAAAGTTAATGGACGGATCTTCGCTAAGCTGACAAAGAA | |
| | GTTAAGGAACTGTCAGGTGATGGCGGAAGGGATCCTAGCCGATCAGATTGCA | |
| | CCTTTCTTTCAGGGAAATGGAGTCATTCAGGATAGCATATCCTTGACCAAGAGT | |
| | ATGCTAGCGATGAGTCAACTGTCTTTTAACAGCAATAAGAAACGTATCACTGA | |
| | CTGTAAAGAAAGAGTATCTTCAAACCGCAATCATGATCCGAAAAGCAAGAACC | |
| | GTCGGAGAGTTGCAACCTTCATAACAACTGACCTGCAAAAGTACTGTCTTAATT | |
| | GGAGATATCAGACAATCAAATTGTTCGCTCATGCCATCAATCAGTTGATGGGC | |
| | CTACCTCACTTCTTCGAATGGATTCACCTAAGACTGATGGACACTACGATGTTC | |
| | GTAGGAGACCCTTTCAATCCTCCAAGTGACCCTACTGACTGTGACCTCTCAAGA | |
| | GTCCCTAATGATGACATATATATTGTCAGTGCCAGAGGGGGTATCGAAGGATT | |
| | ATGCCAGAAGCTATGGACAATGATCTCAATTGCTGCAATCCAACTTGCTGCAGC | |
| | TAGATCGCATTGTCGTGTTGCCTGTATGGTACAGGGTGATAATCAAGTAATAG | |
| | CAGTAACGAGAGAGGTAAGATCAGACGACTCTCCGGAGATGGTGTTGACACA | |
| | GTTGCATCAAGCCAGTGATAATTTCTTCAAGGAATTAATTCATGTCAATCATTT | |
| | GATTGGCCATAATTTGAAGGATCGTGAAACCATCAGGTCAGACACATTCTTCAT | |
| | ATACAGCAAACGAATCTTCAAAGATGGAGCAATCCTCAGTCAAGTCCTCAAAA | |
| | ATTCATCTAAATTAGTGCTAGTGTCAGGTGATCTCAGTGAAAACACCGTAATGT | |
| | CCTGTGCCAACATTGCCTCTACTGTAGCACGGCTATGCGAGAACGGGCTTCCCA | |
| | AAGACTTCTGTTACTATTTAAACTATATAATGAGTTGTGTGCAGACATACTTTG | |
| | ACTCTGAGTTCTCCATCACCAACAATTCGCACCCCGATCTTAATCAGTCGTGGAT | |
| | TGAGGACATCTCTTTTGTGCACTCATATGTTCTGACTCCTGCCCAATTAGGGGG | |
| | ACTGAGTAACCTTCAATACTCAAGGCTCTACACTAGAAATATCGGTGACCCGG | |
| | GGACTACTGCTTTTGCAGAGATCAAGCGACTAGAAGCAGTGGGATTACTGAGT | |
| | CCTAACATTATGACTAATATCTTAACTAGGCCGCCTGGGAATGGAGATTGGGC | |
| | CAGTCTGTGCAACGACCCATACTCTTTCAATTTTGAGACTGTTGCAAGCCCAAA | |
| | TATTGTTCTTAAGAAACATACGCAAAGAGTCCTATTTGAAACTTGTTCAAATCC | |
| | CTTATTGTCTGGAGTGCACACAGAGGATAATGAGGCAGAAGAGAAGGCATTG | |
| | GCTGAATTCTTGCTTAATCAAGAGGTGATTCATCCCCGCGTTGCGCATGCCATC | |
| | ATGGAGGCAAGCTCTGTAGGTAGGAGAAAGCAAATTCAAGGGCTTGTTGACA | |
| | CAACAAACACCGTAATTAAGATTGCGCTTACTAGGAGGCCATTAGGCATCAAG | |
| | AGGCTGATGCGGATAGTCAATTATTCTAGCATGCATGCAATGCTGTTTAGAGA | |
| | CGATGTTTTTTCCTCCAGTAGATCCAACCACCCCTTAGTCTCTTCTAATATGTGTT | |
| | CTCTGACACTGGCAGACTATGCACGGAATAGAAGCTGGTCACCTTTGACGGGA | |
| | GGCAGGAAAATACTGGGTGTATCTAATCCTGATACGATAGAACTCGTAGAGG | |
| | GTGAGATTCTTAGTGTAAGCGGAGGGTGTACAAGATGTGACAGCGGAGATGA | |
| | ACAATTTACTTGGTTCCATCTTCCAAGCAATATAGAATTGACCGATGACACCAG | |
| | CAAGAATCCTCCGATGAGGGTACCATATCTCGGGTCAAAGACACAGGAGAGG | |
| | AGAGCTGCCTCACTTGCAAAAATAGCTCATATGTCGCCACATGTAAAGGCTGCC | |
| | CTAAGGGCATCATCCGTGTTGATCTGGGCTTATGGGGATAATGAAGTAAATTG | |
| | GACTGCTGCTCTTACGATTGCAAAATCTCGGTGTAATGTAAACTTAGAGTATCT | |
| | TCGGTTACTGTCCCCTTTACCCACGGCTGGGAATCTTCAACATAGACTAGATGA | |
| | TGGTATAACTCAGATGACATTCACCCCTGCATCTCTCTACAGGGTGTCACCTTAC | |
| | ATTCACATATCCAATGATTCTCAAAGGCTGTTCACTGAAGAAGGAGTCAAAGA | |
| | GGGGAATGTGGTTTACCAACAGATCATGCTCTTGGGTTTATCTCTAATCGAATC | |
| | GATCTTTCCAATGACAACAACCAGGACATATGATGAGATCACACTGCACCTACA | |
| | TAGTAAATTTAGTTGCTGTATCAGAGAAGCACCTGTTGCGGTTCCTTTCGAGCT | |
| | ACTTGGGGTGGTACCGGAACTGAGGACAGTGACCTCAAATAAGTTTATGTATG | |
| | ATCCTAGCCCTGTATCGGAGGGAGACTTTGCGAGACTTGACTTAGCTATCTTCA | |
| | AGAGTTATGAGCTTAATCTGGAGTCATATCCCACGATAGAGCTAATGAACATT | |
| | CTTTCAATATCCAGCGGGAAGTTGATTGGCCAGTCTGTGGTTTCTTATGATGAA | |
| | GATACCTCCATAAAGAATGACGCCATAATAGTGTATGACAATACCCGAAATTG | |
| | GATCAGTGAAGCTCAGAATTCAGATGTGGTCCGCCTATTTGAATATGCAGCACT | |
| | TGAAGTGCTCCTCGACTGTTCTTACCAACTCTATTACCTGAGAGTAAGAGGCCT | |
| | GGACAATATTGTCTTATATATGGGTGATTTATACAAGAATATGCCAGGAATTCT | |
| | ACTTTCCAACATTGCAGCTACAATATCTCATCCCGTCATTCATTCAAGGTTACAT | |
| | GCAGTGGGCCTGGTCAACCATGACGGATCACACCAACTTGCAGATACGGATTT | |
| | TATCGAAATGTCTGCAAAACTATTAGTATCTTGCACCCGACGTGTGATCTCCGG | |
| | CTTATATTCAGGAAATAAGTATGATCTGCTGTTCCCATCTGTCTTAGATGATAA | |
| | CCTGAATGAGAAGATGCTTCAGCTGATATCCCGGTTATGCTGTCTGTACACGGT | |
| | ACTCTTTGCTACAACAAGAGAAATCCCGAAAATAAGAGGCTTAACTGCAGAAG | |
| | AGAAATGTTCAATACTCACTGAGTATTTACTGTCGGATGCTGTGAAACCATTAC | |
| | TTAGCCCCGATCAAGTGAGCTCTATCATGTCTCCTAACATAATTACATTCCCAGC | |
| | TAATCTGTACTACATGTCTCGGAAGAGCCTCAATTTGATCAGGGAAAGGGAGG | |
| | ACAGGGATACTATCCTGGCGTTGTTGTTCCCCCAAGAGCCATTATTAGAGTTCC | |
| | CTTCTGTGCAAGATATTGGTGCTCGAGTGAAAGATCCATTCACCCGACAACCTG | |
| | CGGCATTTTTGCAAGAGTTAGATTTGAGTGCTCCAGCAAGGTATGACGCATTCA | |
| | CACTTAGTCAGATTCATCCTGAACTCACATCTCCAAATCCGGAGGAAGACTACT | |
| | TAGTACGATACTTGTTCAGAGGGATAGGGACTGCATCTTCCTCTTGGTATAAGG | |
| | CATCTCATCTCCTTTCTGTACCCGAGGTAAGATGTGCAAGACACGGGAACTCCT | |
| | TATACTTAGCTGAAGGGAGCGGAGCCATCATGAGTCTTCTCGAACTGCATGTA | |
| | CCACATGAAACTATCTATTACAATACGCTCTTTTCAAATGAGATGAACCCCCCG | |
| | CAACGACATTTCGGGCCGACCCCAACTCAGTTTTTGAATTCGGTTGTTTATAGG | |
| | AATCTACAGGCGGAGGTAACATGCAAAGATGGATTTGTCCAAGAGTTCCGTCC | |
| | ATTATGGAGAGAAAATACAGAGGAAAGCGACCTGACCTCAGATAAAGTAGTG | |
| | GGGTATATTACATCTGCAGTGCCCTACAGATCTGTATCATTGCTGCATTGTGAC | |
| | ATTGAAATTCCTCCAGGGTCCAATCAAAGCTTACTAGATCAACTAGCTATCAAT | |
| | TTATCTCTGATTGCCATGCATTCTGTAAGGGAGGGCGGGGTAGTAATCATCAA | |
| | AGTGTTGTATGCAATGGGATACTACTTTCATCTACTCATGAACTTGTTTGCTCCG | |
| | TGTTCCACAAAAGGATATATTCTCTCTAATGGTTATGCATGTCGAGGAGATATG | |
| | GAGTGTTACCTGGTATTTGTCATGGGTTACCTGGGCGGGCCTACATTTGTACAT | |
| | GAGGTGGTGAGGATGGCGAAAACTCTGGTGCAGCGGCACGGTACGCTTTTGT | |
| | CTAAATCAGATGAGATCACACTGACCAGGTTATTCACCTCACAGCGGCAGCGT | |
| | GTGACAGACATCCTATCCAGTCCTTTACCAAGATTAATAAAGTACTTGAGGAA | |
| | GAATATTGACACTGCGCTGATTGAAGCCGGGGGACAGCCCGTCCGTCCATTCT | |
| | GTGCGGAGAGTCTGGTGAGCACGCTAGCGAACATAACTCAGATAACCCAGATC | |
| | ATCGCTAGTCACATTGACACAGTTATCCGGTCTGTGATATATATGGAAGCTGAG | |
| | GGTGATCTCGCTGACACAGTATTTCTATTTACCCCTTACAATCTCTCTACTGACG | |
| | GGAAAAAGAGGACATCACTTAAACAGTGCACGAGACAGATCCTAGAGGTTAC | |
| | AATACTAGGTCTTAGAGTCGAAAATCTCAATAAAATAGGCGATATAATCAGCC | |
| | TAGTGCTTAAAGGCATGATCTCCATGGAGGACCTTATCCCACTAAGGACATACT | |
| | TGAAGCATAGTACCTGCCCTAAATATTTGAAGGCTGTCCTAGGTATTACCAAAC | |
| | TCAAAGAAATGTTTACAGACACTTCTGTACTGTACTTGACTCGTGCTCAACAAA | |
| | AATTCTACATGAAAACTATAGGCAATGCAGTCAAAGGATATTACAGTAACTGT | |
| | GACTCTTAACGAAAATCACATATTAATAGGCTCCTTTTTTGGCCAATTGTATTCT | |
| | TGTTGATTTAATCATATTATGTTAGAAAAAAGTTGAACCCTGACTCCTTAGGAC | |
| | TCGAATTCGAACTCAAATAAATGTCTTAAAAAAAGGTTGCGCACAATTATTCTT | |
| | GAGTGTAGTCTCGTCATTCACCAAATCTTTGTTTGGTGCGCGCGGCCGGCATGG | |
| | TCCCAGCCTCCTCGCTGGCGCCGGCTGGGCAACATTCCGAGGGGACCGTCCCCT | |
| | CGGTAATGGCGAATGGGACGTCGACTGCTAACAAAGCCCGAAAGGAAGCTGAG | |
| | TTGGCTGCTGCCACCGCTGAGCAATAACTAGCATAACCCCTTGGGGCCTCTAAAC | |
| | GGGTCTTGAGGGGTTTTTTGCTGAAAGGAGGAACTATATGCGCGCAGATCTGTC | |
| | ATGATGATCATTGCAATTGGATCCATATATAGGGCCCGGGTTATAATTACCTCAG | |
| | GTCGACGTCCCATGGCCATTCGAATTCGTAATCATGGTCATAGCTGTTTCCTGTG | |
| | TGAAATTGTTATCCGCTCACAATTCCACACAACATACGAGCCGGAAGCATAAAGT | |
| | GTAAAGCCTGGGGTGCCTAATGAGTGAGCTAACTCACATTAATTGCGTTGCGCT | |
| | CACTGCCCGCTTTCCAGTCGGGAAACCTGTCGTGCCAGCTGCATTAATGAATCGG | |
| | CCAACGCGCGGGGAGAGGCGGTTTGCGTATTGGGCGC | |
| Additional | GTCGACATTTTTAATTAAAATAGGGTGGGGAAGGTACCGCCACCATGCACCTGC | 89 |
| transcription | TGTGCTTCCTGAGCCTGGCCTGCAGCCTGCTGGCCGCCGCCCTGATCCCCAGCC | |
| unit | CCAGAGAGGCCCCCGCCACCGTGGCCGCCTTCGAGAGCGGCCTGGGCTTCAGC | |
| encoding | GAGGCCGAGCCCGACGGCGGCGAGGTGAAGGCCTTCGAGGGCAAGGACCTG | |
| codon | GAGGAGCAGCTGAGAAGCGTGAGCAGCGTGGACGAGCTGATGAGCGTGCTG | |
| optimized | TACCCCGACTACTGGAAGATGTACAAGTGCCAGCTGAGAAAGGGCGGCTGGC | |
| mVEGF-C | AGCAGCCCACCCTGAACACCAGAACCGGCGACAGCGTGAAGTTCGCCGCCGCC | |
| with APMV | CACTACAACACCGAGATCCTGAAGAGCATCGACAACGAGTGGAGAAAGACCC | |
| gene end, | AGTGCATGCCCAGAGAGGTGTGCATCGACGTGGGCAAGGAGTTCGGCGCCGC | |
| intergenic | CACCAACACCTTCTTCAAGCCCCCCTGCGTGAGCGTGTACAGATGCGGCGGCTG | |
| and gene | CTGCAACAGCGAGGGCCTGCAGTGCATGAACACCAGCACCGGCTACCTGAGCA | |
| start | AGACCCTGTTCGAGATCACCGTGCCCCTGAGCCAGGGCCCCAAGCCCGTGACC | |
| sequences | ATCAGCTTCGCCAACCACACCAGCTGCAGATGCATGAGCAAGCTGGACGTGTA | |
| and Kozak | CAGACAGGTGCACAGCATCATCAGAAGAAGCCTGCCCGCCACCCTGCCCCAGT | |
| sequence, | GCCAGGCCGCCAACAAGACCTGCCCCACCAACTACGTGTGGAACAACTACATG | |
| (size =1302 | TGCAGATGCCTGGCCCAGCAGGACTTCATCTTCTACAGCAACGTGGAGGACGA | |
| nt, rule of 6 | CAGCACCAACGGCTTCCACGACGTGTGCGGCCCCAACAAGGAGCTGGACGAG | |
| OK). | GACACCTGCCAGTGCGTGTGCAAGGGCGGCCTGAGACCCAGCAGCTGCGGCCC | |
| Flanking Sal | CCACAAGGAGCTGGACAGAGACAGCTGCCAGTGCGTGTGCAAGAACAAGCTG | |
| I sites | TTCCCCAACAGCTGCGGCGCCAACAGAGAGTTCGACGAGAACACCTGCCAGTG | |
| underlined. | CGTGTGCAAGAGAACCTGCCCCAGAAACCAGCCCCTGAACCCCGGCAAGTGCG | |
| Open reading | CCTGCGAGTGCACCGAGAACACCCAGAAGTGCTTCCTGAAGGGCAAGAAGTTC | |
| frame in | CACCACCAGACCTGCAGCTGCTACAGAAGACCCTGCGCCAACAGACTGAAGCA | |
| bold. | CTGCGACCCCGGCCTGAGCTTCAGCGAGGAGGTGTGCAGATGCGTGCCCAGCT | |
| | ACTGGAAGAGACCCCACCTGAACTGACCGGGTCGAC | |
| pRz- | TCTTCCGCTTCCTCGCTCACTGACTCGCTGCGCTCGGTCGTTCGGCTGCGGCG | 90 |
| APMV4- | AGCGGTATCAGCTCACTCAAAGGCGGTAATACGGTTATCCACAGAATCAGGGG | |
| mVEGF-C | ATAACGCAGGAAAGAACATGTGAGCAAAAGGCCAGCAAAAGGCCAGGAACCGT | |
| Rescue | AAAAAGGCCGCGTTGCTGGCGTTTTTCCATAGGCTCCGCCCCCCTGACGAGCA | |
| plasmid | TCACAAAAATCGACGCTCAAGTCAGAGGTGGCGAAACCCGACAGGACTATAAA | |
| containing | GATACCAGGCGTTTCCCCCTGGAAGCTCCCTCGTGCGCTCTCCTGTTCCGACC | |
| the full- | CTGCCGCTTACCGGATACCTGTCCGCCTTTCTCCCTTCGGGAAGCGTGGCGCT | |
| length cDNA | TTCTCATAGCTCACGCTGTAGGTATCTCAGTTCGGTGTAGGTCGTTCGCTCCAA | |
| of APMV4 | GCTGGGCTGTGTGCACGAACCCCCCGTTCAGCCCGACCGCTGCGCCTTATCC | |
| with an | GGTAACTATCGTCTTGAGTCCAACCCGGTAAGACACGACTTATCGCCACTGGC | |
| additional | AGCAGCCACTGGTAACAGGATTAGCAGAGCGAGGTATGTAGGCGGTGCTACA | |
| transcription | GAGTTCTTGAAGTGGTGGCCTAACTACGGCTACACTAGAAGAACAGTATTTGGT | |
| unit | ATCTGCGCTCTGCTGAAGCCAGTTACCTTCGGAAAAAGAGTTGGTAGCTCTTGA | |
| encoding a | TCCGGCAAACAAACCACCGCTGGTAGCGGTGGTTTTTTTGTTTGCAAGCAGCA | |
| codon- | GATTACGCGCAGAAAAAAACGATCTCAAGAAGATCCTTTGATCTTTTCTACGGG | |
| optimized | GTCTGACGCTCAGTGGAACGAAAACTCACGTTAAGGGATTTTGGTCATGAGATT | |
| mVEGF-C. | ATCAAAAAGGATCTTCACCTAGATCCTTTTAAATTAAAAATGAAGTTTTAAATCAA | |
| (Additional | TCTAAAGTATATATGAGTAAACTTGGTCTGACAGTTACCAATGCTTAATCAGTGA | |
| transcription | GGCACCTATCTCAGCGATCTGTCTATTTCGTTCATCCATAGTTGCCTGACTCCC | |
| unit in bold.) | CGTCGTGTAGATAACTACGATACGGGAGGGCTTACCATCTGGCCCCAGTGCTG | |
| | CAATGATACCGCGAGACCCACGCTCACCGGCTCCAGATTTATCAGCAATAAAC | |
| | CAGCCAGCCGGAAGGGCCGAGCGCAGAAGTGGTCCTGCAACTTTATCCGCCT | |
| | CCATCCAGTCTATTAATTGTTGCCGGGAAGCTAGAGTAAGTAGTTCGCCAGTTA | |
| | ATAGTTTGCGCAACGTTGTTGCCATTGCTACAGGCATCGTGGTGTCACGCTCG | |
| | TCGTTTGGTATGGCTTCATTCAGCTCCGGTTCCCAACGATCAAGGCGAGTTACA | |
| | TGATCCCCCATGTTGTGCAAAAAAGCGGTTAGCTCCTTCGGTCCTCCGATCGTT | |
| | GTCAGAAGTAAGTTGGCCGCAGTGTTATCACTCATGGTTATGGCAGCACTGCA | |
| | TAATTCTCTTACTGTCATGCCATCCGTAAGATGCTTTTCTGTGACTGGTGAGTAC | |
| | TCAACCAAGTCATTCTGAGAATAGTGTATGCGGCGACCGAGTTGCTCTTGCCC | |
| | GGCGTCAATACGGGATAATACCGCGCCACATAGCAGAACTTTAAAAGTGCTCA | |
| | TCATTGGAAAACGTTCTTCGGGGCGAAAACTCTCAAGGATCTTACCGCTGTTGA | |
| | GATCCAGTTCGATGTAACCCACTCGTGCACCCAACTGATCTTCAGCATCTTTTA | |
| | CTTTCACCAGCGTTTCTGGGTGAGCAAAAACAGGAAGGCAAAATGCCGCAAAA | |
| | AAGGGAATAAGGGCGACACGGAAATGTTGAATACTCATACTCTTCCTTTTTCAA | |
| | TATTATTGAAGCATTTATCAGGGTTATTGTCTCATGAGCGGATACATATTTGAAT | |
| | GTATTTAGAAAAATAAACAAATAGGGGTTCCGCGCACATTTCCCCGAAAAGTGC | |
| | CACCTGACGTCTAAGAAACCATTATTATCATGACATTAACCTATAAAAATAGGCG | |
| | TATCACGAGGCCCTTTCGTCTCGCGCGTTTCGGTGATGACGGTGAAAACCTCT | |
| | GACACATGCAGCTCCCGGAGACGGTCACAGCTTGTCTGTAAGCGGATGCCGG | |
| | GAGCAGACAAGCCCGTCAGGGCGCGTCAGCGGGTGTTGGCGGGTGTCGGGG | |
| | CTGGCTTAACTATGCGGCATCAGAGCAGATTGTACTGAGAGTGCACCATAAAAT | |
| | TGTAAACGTTAATATTTTGTTAAAATTCGCGTTAAATTTTTGTTAAATCAGCTCAT | |
| | TTTTTAACCAATAGGCCGAAATCGGCAAAATCCCTTATAAATCAAAAGAATAGCC | |
| | CGAGATAGGGTTGAGTGTTGTTCCAGTTTGGAACAAGAGTCCACTATTAAAGAA | |
| | CGTGGACTCCAACGTCAAAGGGCGAAAAACCGTCTATCAGGGCGATGGCCCA | |
| | CTACGTGAACCATCACCCAAATCAAGTTTTTTGGGGTCGAGGTGCCGTAAAGC | |
| | ACTAAATCGGAACCCTAAAGGGAGCCCCCGATTTAGAGCTTGACGGGGAAAGC | |
| | CGGCGAACGTGGCGAGAAAGGAAGGGAAGAAAGCGAAAGGAGCGGGCGCTA | |
| | GGGCGCTGGCAAGTGTAGCGGTCACGCTGCGCGTAACCACCACACCCGCCGC | |
| | GCTTAATGCGCCGCTACAGGGCGCGTACTATGGTTGCTTTGACGTATGCGGTG | |
| | TGAAATACCGCACAGATGCGTAAGGAGAAAATACCGCATCAGGCGCCATTCGC | |
| | CATTCAGGCTGCGCAACTGTTGGGAAGGGCGATCGGTGCGGGCCTCTTCGCT | |
| | ATTACGCCAGCTGGCGAAAGGGGGATGTGCTGCAAGGCGATTAAGTTGGGTAA | |
| | CGCCAGGGTTTTCCCAGTCACGACGTTGTAAAACGACGGCCAGTGCCAAGCTT | |
| | TAATACGACTCACTATAGGGAGATTGGTCTGATGAGTCCGTGAGGACGAAACG | |
| | GAGTCTAGACTCCGTCACGAAAAAGAAGAATAAAAGGCAGAAGCCTTTTAAAAG | |
| | GAACCCTGGGCTGTCGTAGGTGTGGGAAGGTTGTATTCCGAGTGCGCCTCCG | |
| | AGGCATCTACTCTACACCTATCACAATGGCTGGTGTCTTCTCCCAGTATGAGAG | |
| | GTTTGTGGACAATCAATCCCAAGTGTCAAGGAAGGATCATCGGTCCTTAGCAG | |
| | GAGGATGCCTTAAAGTTAACATCCCTATGCTTGTCACTGCATCTGAAGACCCCA | |
| | CCACTCGTTGGCAACTAGCATGCTTATCTCTAAGGCTCCTGATCTCCAACTCAT | |
| | CAACCAGTGCTATCCGTCAGGGGGCAATACTGACTCTCATGTCATTACCATCAC | |
| | AAAACATGAGAGCAACAGCAGCTATTGCTGGTTCCACAAATGCAGCTGTTATCA | |
| | ACACCATGGAAGTCTTAAGTGTCAACGACTGGACCCCATCCTTCGACCCTAGG | |
| | AGCGGTCTTTCTGAGGAAGATGCTCAAGTTTTCAGAGACATGGCAAGAGATCT | |
| | GCCCCCTCAGTTCACCTCTGGATCACCCTTCACATCAGCATTGGCGGAGGGGT | |
| | TCACTCCTGAAGATACTCATGACCTGATGGAGGCCTTGACCAGTGTGCTGATA | |
| | CAGATCTGGATCCTGGTGGCTAAGGCCATGACCAACATTGACGGCTCTGGGGA | |
| | GGCCAATGAAAGACGTCTTGCAAAGTACATCCAAAAAGGACAGCTTAATCGTCA | |
| | GTTTGCAATTGGTAATCCTGCCCGTCTGATAATCCAACAGACAATCAAAAGCTC | |
| | CTTAACTGTCCGTAGGTTCTTGGTCTCTGAGCTTCGTGCGTCACGAGGTGCAG | |
| | TAAAAGAAGGATCCCCTTACTATGCAGCTGTTGGGGATATCCACGCTTACATCT | |
| | TTAATGCGGGATTGACACCATTCTTGACCACCTTAAGATACGGGATAGGCACCA | |
| | AGTACGCCGCTGTTGCACTCAGTGTGTTCGCTGCAGATATTGCAAAGTTGAAG | |
| | AGCCTACTTACCCTGTACCAGGACAAGGGTGTAGAAGCTGGATACATGGCACT | |
| | CCTTGAGGATCCAGACTCCATGCACTTTGCACCTGGAAACTTCCCACACATGTA | |
| | CTCCTATGCAATGGGGGTAGCTTCTTACCATGATCCTAGCATGCGCCAATACCA | |
| | ATACGCCAGGAGGTTCCTCAGCCGTCCTTTCTACTTACTAGGAAGGGACATGG | |
| | CCGCCAAGAACACAGGCACGCTGGATGAGCAACTGGCGAAGGAACTGCAAGT | |
| | ATCAGAGAGAGATCGCGCCGCATTATCCGCTGCGATTCAATCAGCGATGGAGG | |
| | GGGGAGAGTCCGACGACTTCCCACTGTCGGGATCCATGCCGGCTCTCTCTGA | |
| | GAATGCGCAACCAGTTACCCCCAGACCTCAACAGTCCCAGCTCTCTCCCCCCC | |
| | AATCATCAAACATGCCCCAATCAGCACCCAGGACCCCAGACTATCAACCCGAC | |
| | TTTGAACTGTAGGCTTCATCACCGCACCAACAACAGCCCAAGAAGACCACCCC | |
| | TCCCCCCACACATCTCACCCAGCCACCCATAAAGACTCAGTGGCGCGCCCCAG | |
| | CATCTCCTTCATTTAATTAAAAACCGACCAACAGGGTGGGGAAGGAGAGTCATT | |
| | GGCTACTGCCAATTGTGTGCAGCAATGGATTTTACTGACATTGATGCTGTCAAC | |
| | TCATTGATCGAATCATCATCGGCAATCATAGACTCCATACAGCATGGAGGGCTG | |
| | CAACCAGCGGGCACCGTCGGCCTATCGCAGATCCCAAAAGGGATAACCAGCG | |
| | CATTAACCAAGGCCTGGGAGGCTGAGGCGGCAACTGCCGGTAATGGGGACAC | |
| | CCAACACAAATCTGACAGTCCGGAGGATCATCAGGCCAACGACACAGATTCCC | |
| | CTGAAGACACAGGTACTGACCAGACCACCCAGGAGGCCAACATCGTTGAGACA | |
| | CCCCACCCCGAGGTGCTGTCAGCAGCCAAAGCCAGACTCAAGAGGCCCAAAG | |
| | CAGGGAGGGACACCCGCGACAACTCCCCTGCGCAACCCGATCATCTTTTAAG | |
| | GGGGGCCTCCTGAGCCCACAACCAGCAGCATCATGGGTGCAAAATCCACCCA | |
| | GTCATGGAGGTCCCGGCACCGCCGATCCCCGCCCATCACAAACTCAGGATCAT | |
| | TCCCCCACCGGAGAGAAATGGCGATTGTCACCGACAAAGCAACCGGAGACATT | |
| | GAACTGGTGGAGTGGTGCAACCCGGGGTGCACAGCAGTCCGAATTGAACCCA | |
| | CCAGACTCGACTGTGTATGCGGACACTGCCCCACCATCTGTAGCCTCTGCATG | |
| | TATGACGACTGATCAGGTACAACTACTAATGAAGGAGGTTGCTGACATAAAATC | |
| | ACTCCTTCAGGCGTTAGTGAGGAACCTCGCTGTCTTGCCCCAATTGAGGAATG | |
| | AGGTTGCAGCAATCAGAACATCACAGGCCATGATAGAGGGGACACTCAATTCG | |
| | ATCAAGATTCTTGACCCTGGGAATTATCAGGAATCATCACTAAACAGTTGGTTC | |
| | AAACCTCGCCAAGATCACACTGTTGTTGTGTCTGGACCAGGGAATCCATTGGC | |
| | CATGCCAACCCCAGTCCAAGACAACACCATATTCCTGGACGAGCTAGCCAGAC | |
| | CTCATCCTAGTGTGGTCAATCCATCCCCGCCCATCACCAACACCAATGTTGACC | |
| | TTGGCCCACAGAAGCAGGCTGCAATAGCCTATATCTCCGCTAAATGCAAGGAT | |
| | CCGGGGAAACGAGATCAGCTATCAAGGCTCATTGAGCGAGCAACCACCCCAA | |
| | GTGAGATCAACAAAGTTAAAAGACAAGCCCTTGGGCTCTAGATCACTCGATCAC | |
| | CCCTCATGGTGATCACAACAATAATCAGAACCCTTCCGAACCACATGACCAACC | |
| | CAGCCCACCGCCCACACCGTCCGTCGACATTTTTAATTAAAATAGGGTGGGG | |
| | AAGGTACCGCCACCATGCACCTGCTGTGCTTCCTGAGCCTGGCCTGCAGCCT | |
| | GCTGGCCGCCGCCCTGATCCCCAGCCCCAGAGAGGCCCCCGCCACCGTGGC | |
| | CGCCTTCGAGAGCGGCCTGGGCTTCAGCGAGGCCGAGCCCGACGGCGGCG | |
| | AGGTGAAGGCCTTCGAGGGCAAGGACCTGGAGGAGCAGCTGAGAAGCGTGA | |
| | GCAGCGTGGACGAGCTGATGAGCGTGCTGTACCCCGACTACTGGAAGATGT | |
| | ACAAGTGCCAGCTGAGAAAGGGCGGCTGGCAGCAGCCCACCCTGAACACCA | |
| | GAACCGGCGACAGCGTGAAGTTCGCCGCCGCCCACTACAACACCGAGATCC | |
| | TGAAGAGCATCGACAACGAGTGGAGAAAGACCCAGTGCATGCCCAGAGAGG | |
| | TGTGCATCGACGTGGGCAAGGAGTTCGGCGCCGCCACCAACACCTTCTTCAA | |
| | GCCCCCCTGCGTGAGCGTGTACAGATGCGGCGGCTGCTGCAACAGCGAGGG | |
| | CCTGCAGTGCATGAACACCAGCACCGGCTACCTGAGCAAGACCCTGTTCGAG | |
| | ATCACCGTGCCCCTGAGCCAGGGCCCCAAGCCCGTGACCATCAGCTTCGCC | |
| | AACCACACCAGCTGCAGATGCATGAGCAAGCTGGACGTGTACAGACAGGTG | |
| | CACAGCATCATCAGAAGAAGCCTGCCCGCCACCCTGCCCCAGTGCCAGGCC | |
| | GCCAACAAGACCTGCCCCACCAACTACGTGTGGAACAACTACATGTGCAGAT | |
| | GCCTGGCCCAGCAGGACTTCATCTTCTACAGCAACGTGGAGGACGACAGCA | |
| | CCAACGGCTTCCACGACGTGTGCGGCCCCAACAAGGAGCTGGACGAGGACA | |
| | CCTGCCAGTGCGTGTGCAAGGGCGGCCTGAGACCCAGCAGCTGCGGCCCCC | |
| | ACAAGGAGCTGGACAGAGACAGCTGCCAGTGCGTGTGCAAGAACAAGCTGT | |
| | TCCCCAACAGCTGCGGCGCCAACAGAGAGTTCGACGAGAACACCTGCCAGT | |
| | GCGTGTGCAAGAGAACCTGCCCCAGAAACCAGCCCCTGAACCCCGGCAAGT | |
| | GCGCCTGCGAGTGCACCGAGAACACCCAGAAGTGCTTCCTGAAGGGCAAGA | |
| | AGTTCCACCACCAGACCTGCAGCTGCTACAGAAGACCCTGCGCCAACAGACT | |
| | GAAGCACTGCGACCCCGGCCTGAGCTTCAGCGAGGAGGTGTGCAGATGCGT | |
| | GCCCAGCTACTGGAAGAGACCCCACCTGAACTGACCGGGTCGACATCCCTTG | |
| | CCAAACATCCTGCCGTAGCTGATTTATTCAAAAGAGCTCATTTGATATGACCTG | |
| | GTAATCATAAAATAGGGTGGGGAAGGTGCTTTGCCTGTAAGGGGGCTCCCTCA | |
| | TCTTCAGACACGTGCCCGCCATCTCACCAACAGTGCAATGGCAGACATGGACA | |
| | CGGTGTATATCAATCTGATGGCAGATGACCCAACCCACCAAAAAGAACTGCTGT | |
| | CCTTTCCTCTCATCCCTGTGACCGGTCCTGACGGGAAGAAGGAACTCCAACAC | |
| | CAGATCCGGACCCAATCCTTGCTCGCCTCAGACAAACAAACTGAACGGTTCAT | |
| | CTTCCTCAACACTTACGGATTCATCTATGACACCACACCGGACAAGACAACTTT | |
| | TTCCACCCCAGAGCATATTAATCAGCCTAAGAGGACGACGGTGAGTGCCGCGA | |
| | TGATGACCATTGGCCTGGTTCCCGCCAATATACCCCTGAACGAACTAACGGCT | |
| | ACTGTGTTCAGCCTTAAAGTAAGAGTGAGGAAAAGTGCGAGGTATCGGGAAGT | |
| | GGTCTGGTATCAATGCAATCCAGTACCGGCCCTGCTTGCAGCCACCAGGTTTG | |
| | GTCGCCAAGGAGGTCTCGAGTCGAGCACTGGAGTCAGTGTAAAGGCTCCCGA | |
| | GAAGATAGATTGTGAGAAGGATTATACCTACTACCCTTATTTCTTATCTGTGTGC | |
| | TACATCGCCACCTCCAACCTGTTCAAGGTACCGAGGATGGTTGCTAATGCAAC | |
| | CAACAGTCAATTATACCACCTTACCATGCAGGTCACATTTGCCTTTCCAAAAAAC | |
| | ATCCCTCCAGCCAACCAGAAACTCCTGACACAGGTGGATGAGGGATTCGAGGG | |
| | CACTGTGGATTGCCATTTTGGGAACATGCTGAAAAAGGATCGGAAAGGGAACA | |
| | TGAGGACACTGTCCCAGGCGGCAGATAAGGTCAGACGAATGAATATTCTTGTT | |
| | GGTATCTTTGACTTGCATGGGCCAACGCTCTTCCTGGAGTATACCGGGAAACT | |
| | GACAAAGGCTCTGCTAGGGTTCATGTCCACCAGCCGAACAGCAATCATCCCCA | |
| | TATCTCAGCTCAATCCCATGCTGAGTCAACTCATGTGGAGCAGTGATGCCCAG | |
| | ATAGTAAAGTTAAGGGTTGTCATAACTACATCCAAACGCGGCCCGTGCGGGGG | |
| | TGAGCAGGAGTATGTGCTGGATCCCAAATTCACAGTTAAGAAAGAAAAGGCTC | |
| | GACTCAACCCTTTCAAGAAGGCAGCCTAATGATTTAATCCGCAAGATCCCAGAA | |
| | ATCAGACCACTCTATACTATCCACTGATCACTGGAAATGTAATCCTGCAGGTGA | |
| | TGAATCTGTGAAGAATCAATTAAAAAACCGGATCCTTATTAGGGTGGGGAAGTA | |
| | GTTGATTGGGTGTCTAAACAAAAGCATTTCTTCACACCTCCCCGCCACGAAACA | |
| | ACCACAATGAGGCTATCAAACACAATCTTGACCTTGATTCTCATCATACTTACCG | |
| | GCTATTTGATAGGTGTCCACTCCACCGATGTGAATGAGAAACCAAAGTCCGAA | |
| | GGGATTAGGGGTGATCTTACACCAGGTGCGGGTATTTTCGTAACTCAAGTCCG | |
| | ACAGCTCCAGATCTACCAACAGTCTGGGTACCATGATCTTGTCATCAGATTGTT | |
| | ACCTCTTCTACCAACAGAGCTTAATGATTGTCAAAGGGAAGTTGTCACAGAGTA | |
| | CAATAACACTGTATCACAGCTGTTGCAGCCTATCAAAACCAACCTGGATACTTT | |
| | GTTGGCAGATGGTAGCACAAGGGATGTTGATATACAGCCGCGATTCATTGGGG | |
| | CAATAATAGCCACAGGTGCCCTGGCTGTAGCAACGGTAGCTGAGGTAACTGCA | |
| | GCTCAAGCACTATCTCAGTCAAAAACGAATGCTCAAAATATTCTCAAGTTGAGA | |
| | GATAGTATTCAGGCCACCAACCAAGCAGTTTTTGAAATTTCACAGGGACTCGAA | |
| | GCAACTGCAACCGTGCTATCAAAACTGCAAACTGAGCTCAATGAGAATATCATC | |
| | CCAAGTCTGAACAACTTGTCCTGTGCTGCCATGGGGAATCGCCTTGGTGTATC | |
| | ACTCTCACTCTATTTGACCTTAATGACCACTCTATTTGGGGACCAGATCACAAA | |
| | CCCAGTGCTGACGCCAATCTCTTACAGCACCCTATCGGCAATGGCGGGTGGTC | |
| | ACATTGGTCCAGTGATGAGTAAGATATTAGCCGGATCTGTCACAAGTCAGTTGG | |
| | GGGCAGAACAACTGATTGCTAGTGGCTTAATACAGTCACAGGTAGTAGGTTAT | |
| | GATTCCCAGTATCAGCTGTTGGTTATCAGGGTCAACCTTGTACGGATTCAGGAA | |
| | GTCCAGAATACTAGGGTTGTATCACTAAGAACACTAGCAGTCAATAGGGATGGT | |
| | GGACTTTACAGAGCCCAGGTGCCACCCGAGGTAGTTGAGCGATCTGGCATTGC | |
| | AGAGCGGTTTTATGCAGATGATTGTGTTCTAACTACAACTGATTACATCTGCTCA | |
| | TCGATCCGATCTTCTCGGCTTAATCCAGAGTTAGTCAAGTGTCTCAGTGGGGCA | |
| | CTTGATTCATGCACATTTGAGAGGGAAAGTGCATTACTGTCAACTCCCTTCTTT | |
| | GTATACAACAAGGCAGTCGTCGCAAATTGTAAAGCAGCGACATGTAGATGTAAT | |
| | AAACCGCCATCTATCATTGCCCAATACTCTGCATCAGCTCTAGTAACCATCACC | |
| | ACCGACACTTGTGCTGACCTTGAAATTGAGGGTTATCGTTTCAACATACAGACT | |
| | GAATCCAACTCATGGGTTGCACCAAACTTCACGGTCTCAACCTCACAAATAGTA | |
| | TCGGTTGATCCAATAGACATATCCTCTGACATTGCCAAAATTAACAATTCTATCG | |
| | AGGCTGCGCGAGAGCAGCTGGAACTGAGCAACCAGATCCTTTCCCGAATCAAC | |
| | CCACGGATTGTGAACGACGAATCACTAATAGCTATTATCGTGACAATTGTTGTG | |
| | CTTAGTCTCCTTGTAATTGGTCTTATTATTGTTCTCGGTGTGATGTACAAGAATC | |
| | TTAAGAAAGTCCAACGAGCTCAAGCTGCTATGATGATGCAGCAAATGAGCTCAT | |
| | CACAGCCTGTGACCACCAAATTGGGGACACCCTTCTAGGTGAATAATCATATCA | |
| | ATCCATTCAATAATGAGCGGGACATACCAATCACCAACGACTGTGTCACAAGGC | |
| | CGGTTAGGAATGCACCGGATCTCTCTCCTTCCTTTTTAATTAAAAACGATCGAA | |
| | CTGAGGGTGAGGGGGGGGGTGTGCATGGTAGGGTGGGGAAGGTAGCCAATT | |
| | CCTGCCCATTGGGCCGACCGTACCAAGAGAAGTCAACAGAAGTATAGATGCAG | |
| | GGCGACATGGAGGGTAGCCGTGATAACCTCACAGTAGATGATGAATTAAAGAC | |
| | AACATGGAGGTTAGCTTATAGAGTTGTATCCCTCCTATTGATGGTGAGTGCCTT | |
| | GATAATCTCTATAGTAATCCTGACGAGAGATAACAGCCAAAGCATAATCACGGC | |
| | GATCAACCAGTCGTATGACGCAGACTCAAAGTGGCAAACAGGGATAGAAGGGA | |
| | AAATCACCTCAATCATGACTGATACGCTCGATACCAGGAATGCAGCTCTTCTCC | |
| | ACATTCCACTCCAGCTCAATACACTTGAGGCAAACCTGTTGTCCGCCCTCGGA | |
| | GGTAACACGGGAATTGGCCCCGGAGATCTAGAGCACTGTCGTTATCCGGTTCA | |
| | TGACTCCGCTTACCTGCATGGAGTCAATCGATTACTCATCAATCAAACAGCTGA | |
| | CTACACAGCAGAAGGCCCCCTGGATCATGTGAACTTCATTCCGGCACCAGTTA | |
| | CGACTACTGGATGCACAAGGATCCCATCCTTTTCTGTATCATCATCCATTTGGT | |
| | GCTATACACACAATGTGATTGAAACAGGTTGCAATGACCACTCAGGTAGTAATC | |
| | AATATATCAGTATGGGGGTGATTAAGAGGGCTGGCAACGGCTTACCTTACTTCT | |
| | CAACAGTCGTGAGTAAGTATCTGACCGATGGGTTGAATAGAAAAAGCTGTTCC | |
| | GTAGCTGCCGGATCCGGGCATTGTTACCTCCTTTGTAGCCTAGTGTCAGAGCC | |
| | CGAACCTGATGACTATGTGTCACCAGATCCCACACCGATGAGGTTAGGGGTGC | |
| | TAACAAGGGATGGGTCTTACACTGAACAGGTGGTACCCGAAAGAATATTTAAGA | |
| | ACATATGGAGCGCAAACTACCCTGGGGTAGGGTCAGGTGCTATAGTAGGAAAT | |
| | AAGGTGTTATTCCCA1111ACGGCGGAGTGAAGAATGGATCAACCCCTGAGGT | |
| | GATGAATAGGGGAAGATATTACTACATCCAGGATCCAAATGACTATTGCCCTGA | |
| | CCCGCTGCAAGATCAGATCTTAAGGGCAGAACAATCGTATTATCCTACTCGATT | |
| | TGGTAGGAGGATGGTAATGCAGGGAGTCCTAACATGTCCAGTATCCAACAATT | |
| | CAACAATAGCCAGCCAATGCCAATCTTACTATTTCAACAACTCATTAGGATTCAT | |
| | CGGGGCGGAATCTAGGATCTATTACCTCAATGGTAACATTTACCTTTATCAAAG | |
| | AAGCTCGAGCTGGTGGCCTCACCCCCAAATTTACCTACTTGATTCCAGGATTGC | |
| | AAGTCCGGGTACGCAGAACATTGACTCAGGCGTTAACCTCAAGATGTTAAATGT | |
| | TACTGTCATTACACGACCATCATCTGGCTTTTGAATAGTCAGTCAAGATGCCC | |
| | TAATGACTGCTTATTCGGGGTTTATTCAGATGTCTGGCCTCTTAGCCTTACCTC | |
| | AGACAGCATATTTGCATTTACAATGTACTTACAAGGGAAGACGACACGTATTGA | |
| | CCCAGCTTGGGCGCTATTCTCCAATCATGTAATTGGGCATGAGGCTCGTTTGTT | |
| | CAACAAGGAGGTTAGTGCTGCTTATTCTACCACCACTTGTTTTTCGGACACCAT | |
| | CCAAAACCAGGTGTATTGTCTGAGTATACTTGAAGTCAGAAGTGAGCTCTTGGG | |
| | GGCATTCAAGATAGTGCCATTCCTCTATCGTGTCTTATAGGCACCTGCTTGGTC | |
| | AAGAACCCTGAGCGGCCGTAAAATTAACACTTGATCTTCCTTAAAAACACCTAT | |
| | CTAAATTACTGTCTGAGATCCCTGATTAGTTACCCTTTCAATCAATCAATTAATTT | |
| | TTAATTAAAAACGGAAAAATGGGCCTAGTTCCAAGGAAAGGATGGGACCCATTA | |
| | GGGTGGGGAAGGATTACTTTGTTCCTTGACTCGCACCCACGTACACCCAATCC | |
| | CATTCCTGTCAAGAAGGAACCCTTCCCAAACTCACCTTGCAATGTCCAATCAGG | |
| | CAGCTGAGATTATACTACCCACCTTCCATCTGGAATCACCCTTGATCGAGAATA | |
| | AGTGCTTCTACTACATGCAATTACTTGGTCTCGTGTTACCACATGATCACTGGA | |
| | GATGGAGGGCATTCGTCAA1111ACAGTGGATCAAGCACACCTTAAAAATCGTA | |
| | ATCCCCGCTTAATGGCCCACATCGATCACACTAAGGATAGACTAAGGGCTCAT | |
| | GGTGTCTTGGGTTTCCACCAGACTCAGACAAGTGAGAGCCGTTTCCGTGTCTT | |
| | GCTCCATCCTGAAACTTTACCTTGGCTATCAGCAATGGGAGGATGCATCAACCA | |
| | GGTTCCCAAGGCATGGCGGAACACTCTGAAATCTATCGAGCACAGTGTGAAGC | |
| | AGGAGGCGACTCAACTGAAGTTACTCATGGAAAAAACCTCACTAAAGCTAACAG | |
| | GAGTATCTTACTTATTCTCCAATTGCAATCCCGGGAAAACTGCAGCGGGAACTA | |
| | TGCCCGTACTAAGTGAGATGGCATCAGAACTCTTGTCAAATCCCATCTCCCAAT | |
| | TCCAATCAACATGGGGGTGTGCTGCTTCAGGGTGGCACCATGTAGTCAGCATC | |
| | ATGAGGCTCCAACAGTATCAAAGAAGGACAGGTAAGGAAGAGAAAGCAATCAC | |
| | TGAAGTTCAGTATGGCTCGGACACCTGTCTCATTAATGCAGACTACACCGTCGT | |
| | TTTTTCCGCACAGGACCGTGTCATAGCAGTCTTGCCTTTCGATGTTGTCCTCAT | |
| | GATGCAAGACCTGCTTGAATCCCGACGGAATGTCTTGTTCTGTGCCCGCTTTAT | |
| | GTATCCCAGAAGCCAACTACATGAGAGGATAAGTACAATACTGGCCCTTGGAG | |
| | ACCAACTCGGGAGAAAAGCACCCCAAGTCCTGTATGATTTCGTAGCTACCCTC | |
| | GAATCATTTGCATACGCTGCTGTCCAACTTCATGACAACAACCCTATCTACGGT | |
| | GGGGCTTTCTTTGAGTTCAATATCCAAGAACTGGAAGCTATTTTGTCCCCTGCA | |
| | CTTAATAAGGATCAAGTCAACTTCTACATAAGTCAAGTTGTCTCAGCATACAGTA | |
| | ACCTTCCCCCATCTGAATCAGCAGAATTGCTATGCTTACTACGCCTGTGGGGTC | |
| | ATCCCTTGCTAAACAGTCTTGATGCAGCAAAGAAAGTCAGAGAATCTATGTGTG | |
| | CTGGGAAGGTTCTTGATTATAATGCTATTCGACTAGTTTTGTCTTTTTATCATAC | |
| | GTTATTAATCAATGGGTATCGGAAGAAACATAAGGGTCGCTGGCCAAATGTGAA | |
| | TCAACATTCACTACTCAACCCGATAGTGAAGCAGCTTTACTTTGATCAGGAGGA | |
| | GATCCCACACTCTGTTGCCCTTGAGCACTATTTAGATATCTCGATGATAGAATTT | |
| | GAGAAGACTTTGAAGTGGAACTATCTGATAGTCTAAGCATCTTTCTGAAGGAT | |
| | AAGTCGATAGCTTTGGATAAACAAGAATGGCACAGTGGTTTTGTCTCAGAAGTG | |
| | ACTCCAAAGCACCTACGAATGTCTCGTCATGATCGCAAGTCTACCAATAGGCTA | |
| | TTGTTAGCCTTTATTAACTCCCCTGAATTCGATGTTAAGGAAGAGCTTAAATATT | |
| | TGACTACAGGTGAGTATGCCACTGACCCAAATTTCAATGTCTCTTACTCACTGA | |
| | AAGAGAAGGAAGTTAAGAAAGAAGGGCGCATTTTCGCAAAGATGTCACAGAAA | |
| | ATGAGAGCATGCCAGGTTATTTGTGAAGAGTTACTAGCACATCATGTGGCTCCT | |
| | TTGTTTAAAGAGAATGGTGTTACACAATCGGAGCTATCCCTGACAAAGAATTTG | |
| | TTGGCTATTAGCCAACTGAGTTACAACTCGATGGCCGCTAAGGTGCGATTGCT | |
| | GAGGCCAGGGGACAAGTTCACCGCTGCACACTATATGACCACAGACCTAAAAA | |
| | AGTACTGCCTTAACTGGCGGCACCAGTCAGTCAAATTGTTCGCCAGAAGCCTG | |
| | GATCGACTATTTGGGTTAGACCATGCTTTTTCTTGGATACACGTCCGTCTCACC | |
| | AATAGCACTATGTACGTTGCTGACCCATTCAATCCACCAGACTCAGATGCATGC | |
| | ACAAATTTAGACGACAATAAGAACACTGGGATTTTTATTATAAGTGCTCGAGGT | |
| | GGTATAGAAGGCCTTCAACAGAAACTATGGACTGGCATATCAATTGCAATCGCC | |
| | CAGGCGGCAGCAGCCCTCGAGGGCTTACGAATTGCTGCCACTTTGCAGGGGG | |
| | ATAACCAGGTTTTAGCGATTACGAAAGAATTCATGACCCCAGTCTCGGAGGATG | |
| | TAATCCACGAGCAGCTATCTGAAGCGATGTCGCGATACAAGAGGACTTTCACAT | |
| | ACCTTAATTATTTAATGGGGCACCAATTGAAGGATAAAGAAACCATCCAATCCA | |
| | GTGACTTCTTCGTTTACTCCAAAAGGATCTTCTTCAATGGGTCAATCCTAAGTCA | |
| | ATGCCTCAAGAACTTCAGTAAACTCACTACCAATGCCACTACCCTTGCTGAGAA | |
| | CACTGTAGCCGGCTGCAGTGACATCTCCTCATGCATAGCCCGTTGTGTGGAAA | |
| | ACGGGTTGCCTAAGGATGCTGCATATGTTCAGAATATAATCATGACTCGGCTTC | |
| | AACTGTTGCTAGATCACTACTATTCTATGCATGGTGGCATAAACTCAGAGTTAG | |
| | AGCAGCCAACTCTAAGTATCCCTGTCCGAAACGCAACCTATTTACCATCTCAAT | |
| | TAGGCGGTTACAATCATTTGAATATGACCCGACTATTCTGTCGCAATATCGGTG | |
| | ACCCGCTTACTAGTTCTTGGGCAGAGTCAAAAAGACTAATGGATGTTGGCCTTC | |
| | TCAGTCGTAAGTTCTTAGAGGGGATATTATGGAGACCCCCGGGAAGTGGGACA | |
| | TTTTCAACACTCATGCTTGATCCGTTCGCACTTAACATTGATTACTTAAGGCCAC | |
| | CAGAGACAATAATCCGAAAACACACCCAAAAAGTCTTGTTGCAGGATTGTCCTA | |
| | ATCCTCTATTAGCAGGTGTAGTTGACCCGAACTACAACCAGGAATTAGAATTAT | |
| | TAGCTCAGTTCCTGCTTGATCGGGAAACCGTTATTCCCAGGGCTGCCCATGCC | |
| | ATCTTTGAACTGTCTGTCTTGGGAAGGAAAAAACATATACAAGGATTGGTTGAT | |
| | ACTACAAAAACAATTATTCAGTGCTCATTAGAAAGACAGCCACTGTCCTGGAGG | |
| | AAAGTTGAGAACATTGTAACCTACAATGCGCAGTATTTCCTCGGGGCCACCCA | |
| | GCAGGTTGACACCAATATCTCAGAAAGGCAGTGGGTGATGCCAGGTAATTTCA | |
| | AGAAGCTTGTATCTCTTGACGATTGCTCAGTCACGTTGTCCACTGTGTCACGGC | |
| | GCATTTCTTGGGCCAATCTACTTAACTGGAGGGCTATAGATGGTTTGGAAACTC | |
| | CAGATGTGATAGAGAGTATTGATGGCCGCCTTGTGCAATCATCCAATCAATGCG | |
| | GCCTATGTAATCAAGGATTGGGCTCCTACTCCTGGTTCTTCTTGCCCTCCGGGT | |
| | GTGTGTTCGACCGTCCACAAGATTCTCGAGTGGTTCCAAAGATGCCATACGTG | |
| | GGATCCAAAACGGATGAGAGACAGACTGCGTCAGTGCAGGCTATACAGGGATC | |
| | CACATGTCACCTTAGAGCAGCATTGAGACTTGTATCACTCTACCTTTGGGCCTA | |
| | TGGAGATTCTGACATATCATGGCTAGAAGCCGCGACATTGGCTCAAACACGGT | |
| | GCAATATTTCTCTTGATGACCTGCGGATCCTGAGCCCTCTTCCTTCCTCGGCAA | |
| | ATTTACACCACAGATTGAATGACGGGGTAACACAAGTGAAATTCATGCCCGCCA | |
| | CATCGAGCCGGGTGTCAAAGTTCGTCCAAATTTGCAATGACAACCAGAATCTTA | |
| | TCCGTGATGATGGGAGTGTTGATTCCAATATGATTTATCAGCAGGTTATGATATT | |
| | AGGGCTTGGAGAGATTGAATGTTTGTTAGCTGACCCAATCGATACAAACCCAGA | |
| | ACAACTGATTCTTCACCTACACTCTGATAATTCTTGCTGTCTCCGGGAGATGCC | |
| | AACGACCGGTTTTGTACCTGCTTTAGGATTGACCCCATGCTTAACTGTCCCAAA | |
| | GCACAATCCGTATATTTATGATGATAGCCCAATACCCGGTGATTTGGATCAGAG | |
| | GCTCATTCAAACCAAATTCTTTATGGGTTCTGACAATCTAGATAATCTTGATATC | |
| | TACCAGCAGCGAGCTTTACTGAGTCGGTGTGTGGCTTATGACATTATCCAATCA | |
| | GTATTCGCTTGCGATGCACCAGTATCTCAGAAGAATGATGCAATCCTTCACACT | |
| | GACTACCATGAAAATTGGATCTCAGAGTTCCGATGGGGTGACCCTCGCATAAT | |
| | CCAAGTAACAGCAGGTTACGAGTTAATTCTGTTCCTTGCATACCAGCTTTATTAT | |
| | CTCAGAGTGAGGGGTGACCGTGCAATCCTGTGTTATATTGATAGGATACTCAAC | |
| | AGGATGGTATCTTCCAATCTAGGCAGTCTCATCCAGACGCTCTCTCATCCGGA | |
| | GATTAGGAGGAGATTTTCATTGAGTGATCAAGGGTTCCTTGTCGAAAGGGAGC | |
| | TAGAGCCAGGTAAGCCACTGGTAAAACAAGCGGTTATGTTCCTAAGGGACTCA | |
| | GTCCGCTGCGCTTTAGCAACTATCAAGGCAGGAATTGAGCCTGAGATCTCCCG | |
| | AGGTGGCTGTACCCAGGATGAGCTGAGCTTTACCCTTAAGCACTTACTATGTC | |
| | GGCGTCTCTGTATAATTGCTCTCATGCATTCGGAAGCAAAGAACTTGGTCAAAG | |
| | TTAGAAACCTTCCAGTAGAGGAAAAAACCGCCTTACTATACCAGATGTTGATCA | |
| | CTGAGGCCAATGCCAGGAGATCAGGGTCTGCTAGTATCATCATAAGCTTAGTTT | |
| | CAGCACCCCAGTGGGACATTCATACACCAGCGTTGTATTTTGTATCAAAGAAAA | |
| | TGCTGGGGATGCTCAAAAGGTCAACCACACCCTTGGATATAAGTGACCTTTCTG | |
| | AGAGCCAGAACCTCACACCAACAGAATTGAATGATGTTCCTGGTCACATGGCA | |
| | GAGGAATTTCCCTGTTTGTTTAGCAGTTATAACGCTACATATGAAGACACAATTA | |
| | CTTACAATCCAATGACTGAAAAACTCGCAGTGCACTTGGACAATGGTTCCACCC | |
| | CTTCCAGAGCGCTTGGTCGTCACTACATCCTGCGACCCCTTGGGCTTTACTCG | |
| | TCTGCATGGTACCGGTCTGCAGCACTATTAGCGTCAGGGGCCCTCAGTGGGTT | |
| | GCCTGAGGGGTCAAGCCTGTACTTGGGAGAGGGGTATGGGACCACCATGACT | |
| | CTACTTGAGCCCGTTGTCAAGTCCTCAACTGTTTACTACCATACATTGTTTGACC | |
| | CAACCCGGAATCCTTCACAGCGGAACTACAAACCAGAACCGCGGGTATTCACT | |
| | GATTCCATTTGGTACAAGGATGATTTCACACGACCACCTGGTGGCATTGTAAAT | |
| | CTATGGGGTGAAGACGTACGTCAGAGTGATATTACACAGAAAGACACGGTTAA | |
| | TTTCATATTATCTCGGGTCCCGCCAAAATCACTCAAATTGATACACGTTGATATT | |
| | GAGTTCTCCCCAGACTCTGATGTACGGACGCTACTATCTGGCTATTCCCATTGT | |
| | GCACTATTGGCCTACTGGCTACTGCAACCTGGAGGGCGATTTGCGGTTAGAGT | |
| | TTTCTTAAGTGACCATATCATAGTCAACTTGGTCACTGCCATTCTGTCCGCTTTT | |
| | GACTCTAATCTGGTGTGCATTGCGTCAGGATTGACACACAAGGATGATGGGGC | |
| | AGGTTATATTTGTGCAAAGAAGCTTGCAAATGTTGAGGCTTCAAGAATTGAGTA | |
| | TTACTTGAGGATGGTCCACGGCTGTGTTGACTCATTAAAAATTCCTCATCAATTA | |
| | GGAATCATTAAATGGGCTGAGGGTGAAGTGTCCCGACTTACCAAAAAGGCGGA | |
| | TGATGAAATAAACTGGCGGTTAGGTGATCCAGTTACCAGATCATTTGATCCGGT | |
| | TTCTGAGCTAATAATTGCGCGAACAGGGGGATCAGTATTAATGGAATACGGGA | |
| | CTTTTACTAACCTCAGGTGTGCGAACTTGGCAGATACATATAAACTTTTGGCTTC | |
| | AATTGTAGAGACCACCTTAATGGAAATAAGGGTTGAGCAAGATCAGTTGGAAGA | |
| | TGATTCGAGGAGACAAATCCAGGTAGTCCCTGC1111AATACAAGATCCGGGG | |
| | GAAGGATCCGTACATTGATTGAGTGTGCTCAGCTGCAGGTCATAGATGTTATCT | |
| | GTGTGAACATAGATCACCTCTTTCCCAAACACCGACATGCTCTTGTCACACAAC | |
| | TTACTTACCAGTCAGTGTGCCTTGGGGACTTGATTGAAGGCCCCCAAATTAAGA | |
| | CATATCTAAGGGCCAGGAAGTGGATCCAACGTAGGGGACTCAATGAGACAATT | |
| | AACCATATCATCACTGGACAAGTGTCGCGGAATAAGGCAAGGGATTTTTTCAAG | |
| | AGGCGCCTGAAGTTGGTTGGCTTTTCGCTCTGTGGCGGTTGGGGCTACCTCTC | |
| | ACTTTAGCTGCTTAGATTGTTGATTATTATGAATAATCGGAGTCGAAATCGTAAA | |
| | TAGAAAGACATAAAATTGCAAATAAGCAATGATCGTATTAATATTTAATAAAAAAT | |
| | ATGTCTTTTATTTCTTGCGCGCGGCCGGCATGGTCCCAGCCTCCTCGCTGGCG | |
| | CCGGCTGGGCAACATTCCGAGGGGACCGTCCCCTCGGTAATGGCGAATGGGA | |
| | CGTCGACTGCTAACAAAGCCCGAAAGGAAGCTGAGTTGGCTGCTGCCACCGCT | |
| | GAGCAATAACTAGCATAACCCCTTGGGGCCTCTAAACGGGTCTTGAGGGGTTT | |
| | TTTGCTGAAAGGAGGAACTATATGCGCGCAGATCTGTCATGATGATCATTGCAA | |
| | TTGGATCCATATATAGGGCCCGGGTTATAATTACCTCAGGTCGACGTCCCATG | |
| | GCCATTCGAATTCGTAATCATGGTCATAGCTGTTTCCTGTGTGAAATTGTTATCC | |
| | GCTCACAATTCCACACAACATACGAGCCGGAAGCATAAAGTGTAAAGCCTGGG | |
| | GTGCCTAATGAGTGAGCTAACTCACATTAATTGCGTTGCGCTCACTGCCCGCTT | |
| | TCCAGTCGGGAAACCTGTCGTGCCAGCTGCATTAATGAATCGGCCAACGCGCG | |
| | GGGAGAGGCGGTTTGCGTATTGGGCGC | |

TABLE 3
VEGF-C/VEGF-D SEQUENCES

| | | SEQ ID |
|---|---|---|
| Description | Sequence | NO. |
| mouse VEGF- | ATGCACTTGCTGTGCTTCTTGTCTCTGGCGTGTTCCCTGCTCG | 1 |
| C full length | CCGCTGCGCTGATCCCCAGTCCGCGCGAGGCGCCCGCCACCG | |
| wt native | TCGCCGCCTTCGAGTCGGGACTGGGCTTCTCGGAAGCGGAGC | |
| | CCGACGGGGGCGAGGTCAAGGCTTTTGAAGGCAAAGACCTG | |
| | GAGGAGCAGTTGCGGTCTGTGTCCAGCGTAGATGAGCTGATG | |
| | TCTGTCCTGTACCCAGACTACTGGAAAATGTACAAGTGCCAG | |
| | CTGCGGAAAGGCGGCTGGCAGCAGCCCACCCTCAATACCAGG | |
| | ACAGGGGACAGTGTAAAATTTGCTGCTGCACATTATAACACA | |
| | GAGATCCTGAAAAGTATTGATAATGAGTGGAGAAAGACTCA | |
| | ATGCATGCCACGTGAGGTGTGTATAGATGTGGGGAAGGAGTT | |
| | TGGAGCAGCCACAAACACCTTCTTTAAACCTCCATGTGTGTCC | |
| | GTCTACAGATGTGGGGGTTGCTGCAACAGCGAGGGGCTGCAG | |
| | TGCATGAACACCAGCACAGGTTACCTCAGCAAGACGTTGTTT | |
| | GAAATTACAGTGCCTCTCTCACAAGGCCCCAAACCAGTCACA | |
| | ATCAGTTTTGCCAATCACACTTCCTGCCGGTGCATGTCTAAAC | |
| | TGGATGTTTACAGACAAGTTCATTCAATTATTAGACGTTCTCT | |
| | GCCAGCAACATTACCACAGTGTCAGGCAGCTAACAAGACATG | |
| | TCCAACAAACTATGTGTGGAATAACTACATGTGCCGATGCCT | |
| | GGCTCAGCAGGATTTTATCTTTTATTCAAATGTTGAAGATGAC | |
| | TCAACCAATGGATTCCATGATGTCTGTGGACCCAACAAGGAG | |
| | CTGGATGAAGACACCTGTCAGTGTGTCTGCAAGGGGGGGCTT | |
| | CGGCCATCTAGTTGTGGACCCCACAAAGAACTAGATAGAGAC | |
| | TCATGTCAGTGTGTCTGTAAAAACAAACTTTTCCCTAATTCAT | |
| | GTGGAGCCAACAGGGAATTTGATGAGAATACATGTCAGTGTG | |
| | TATGTAAAAGAACGTGTCCAAGAAATCAGCCCCTGAATCCTG | |
| | GGAAATGTGCCTGTGAATGTACAGAAAACACACAGAAGTGCT | |
| | TCCTTAAAGGGAAGAAGTTCCACCATCAAACATGCAGTTGTT | |
| | ACAGAAGACCGTGTGCGAATCGACTGAAGCATTGTGATCCAG | |
| | GACTGTCCTTTAGTGAAGAAGTATGCCGCTGTGTCCCATCGTA | |
| | TTGGAAAAGGCCACATCTGAACTAA | |
| mouse VEGF- | ATGCACTTGCTGTGCTTCTTGTCTCTGGCGTGTTCCCTGCTCG | 2 |
| C full length | CCGCTGCGCTGATCCCCAGTCCGCGCGAGGCGCCCGCCACCG | |
| Cys152Ser | TCGCCGCCTTCGAGTCGGGACTGGGCTTCTCGGAAGCGGAGC | |
| mutation | CCGACGGGGGCGAGGTCAAGGCTTTTGAAGGCAAAGACCTG | |
| native | GAGGAGCAGTTGCGGTCTGTGTCCAGCGTAGATGAGCTGATG | |
| | TCTGTCCTGTACCCAGACTACTGGAAAATGTACAAGTGCCAG | |
| | CTGCGGAAAGGCGGCTGGCAGCAGCCCACCCTCAATACCAGG | |
| | ACAGGGGACAGTGTAAAATTTGCTGCTGCACATTATAACACA | |
| | GAGATCCTGAAAAGTATTGATAATGAGTGGAGAAAGACTCA | |
| | ATGCATGCCACGTGAGGTGTGTATAGATGTGGGGAAGGAGTT | |
| | TGGAGCAGCCACAAACACCTTCTTTAAACCTCCATCTGTGTCC | |
| | GTCTACAGATGTGGGGGTTGCTGCAACAGCGAGGGGCTGCAG | |
| | TGCATGAACACCAGCACAGGTTACCTCAGCAAGACGTTGTTT | |
| | GAAATTACAGTGCCTCTCTCACAAGGCCCCAAACCAGTCACA | |
| | ATCAGTTTTGCCAATCACACTTCCTGCCGGTGCATGTCTAAAC | |
| | TGGATGTTTACAGACAAGTTCATTCAATTATTAGACGTTCTCT | |
| | GCCAGCAACATTACCACAGTGTCAGGCAGCTAACAAGACATG | |
| | TCCAACAAACTATGTGTGGAATAACTACATGTGCCGATGCCT | |
| | GGCTCAGCAGGATTTTATCTTTTATTCAAATGTTGAAGATGAC | |
| | TCAACCAATGGATTCCATGATGTCTGTGGACCCAACAAGGAG | |
| | CTGGATGAAGACACCTGTCAGTGTGTCTGCAAGGGGGGGCTT | |
| | CGGCCATCTAGTTGTGGACCCCACAAAGAACTAGATAGAGAC | |
| | TCATGTCAGTGTGTCTGTAAAAACAAACTTTTCCCTAATTCAT | |
| | GTGGAGCCAACAGGGAATTTGATGAGAATACATGTCAGTGTG | |
| | TATGTAAAAGAACGTGTCCAAGAAATCAGCCCCTGAATCCTG | |
| | GGAAATGTGCCTGTGAATGTACAGAAAACACACAGAAGTGCT | |
| | TCCTTAAAGGGAAGAAGTTCCACCATCAAACATGCAGTTGTT | |
| | ACAGAAGACCGTGTGCGAATCGACTGAAGCATTGTGATCCAG | |
| | GACTGTCCTTTAGTGAAGAAGTATGCCGCTGTGTCCCATCGTA | |
| | TTGGAAAAGGCCACATCTGAACTAA | |
| mouse VEGF- | ATGCACTTGCTGTGCTTCTTGTCTCTGGCGTGTTCCCTGCTCG | 3 |
| C full length | CCGCTGCGCTGATCCCCAGTCCGCGCGAGGCGCCCGCCACCG | |
| Cys133Ala | TCGCCGCCTTCGAGTCGGGACTGGGCTTCTCGGAAGCGGAGC | |
| mutation | CCGACGGGGGCGAGGTCAAGGCTTTTGAAGGCAAAGACCTG | |
| native | GAGGAGCAGTTGCGGTCTGTGTCCAGCGTAGATGAGCTGATG | |
| | TCTGTCCTGTACCCAGACTACTGGAAAATGTACAAGTGCCAG | |
| | CTGCGGAAAGGCGGCTGGCAGCAGCCCACCCTCAATACCAGG | |
| | ACAGGGGACAGTGTAAAATTTGCTGCTGCACATTATAACACA | |
| | GAGATCCTGAAAAGTATTGATAATGAGTGGAGAAAGACTCA | |
| | ATGCATGCCACGTGAGGTGGCTATAGATGTGGGGAAGGAGTT | |
| | TGGAGCAGCCACAAACACCTTCTTTAAACCTCCATGTGTGTCC | |
| | GTCTACAGATGTGGGGGTTGCTGCAACAGCGAGGGGCTGCAG | |
| | TGCATGAACACCAGCACAGGTTACCTCAGCAAGACGTTGTTT | |
| | GAAATTACAGTGCCTCTCTCACAAGGCCCCAAACCAGTCACA | |
| | ATCAGTTTTGCCAATCACACTTCCTGCCGGTGCATGTCTAAAC | |
| | TGGATGTTTACAGACAAGTTCATTCAATTATTAGACGTTCTCT | |
| | GCCAGCAACATTACCACAGTGTCAGGCAGCTAACAAGACATG | |
| | TCCAACAAACTATGTGTGGAATAACTACATGTGCCGATGCCT | |
| | GGCTCAGCAGGATTTTATCTTTTATTCAAATGTTGAAGATGAC | |
| | TCAACCAATGGATTCCATGATGTCTGTGGACCCAACAAGGAG | |
| | CTGGATGAAGACACCTGTCAGTGTGTCTGCAAGGGGGGGCTT | |
| | CGGCCATCTAGTTGTGGACCCCACAAAGAACTAGATAGAGAC | |
| | TCATGTCAGTGTGTCTGTAAAAACAAACTTTTCCCTAATTCAT | |
| | GTGGAGCCAACAGGGAATTTGATGAGAATACATGTCAGTGTG | |
| | TATGTAAAAGAACGTGTCCAAGAAATCAGCCCCTGAATCCTG | |
| | GGAAATGTGCCTGTGAATGTACAGAAAACACACAGAAGTGCT | |
| | TCCTTAAAGGGAAGAAGTTCCACCATCAAACATGCAGTTGTT | |
| | ACAGAAGACCGTGTGCGAATCGACTGAAGCATTGTGATCCAG | |
| | GACTGTCCTTTAGTGAAGAAGTATGCCGCTGTGTCCCATCGTA | |
| | TTGGAAAAGGCCACATCTGAACTAA | |
| mouse VEGF- | GCACATTATAACACAGAGATCCTGAAAAGTATTGATAATGAG | 4 |
| C mature | TGGAGAAAGACTCAATGCATGCCACGTGAGGTGTGTATAGAT | |
| (dNdC) wt | GTGGGGAAGGAGTTTGGAGCAGCCACAAACACCTTCTTTAAA | |
| native | CCTCCATGTGTGTCCGTCTACAGATGTGGGGGTTGCTGCAAC | |
| | AGCGAGGGGCTGCAGTGCATGAACACCAGCACAGGTTACCTC | |
| | AGCAAGACGTTGTTTGAAATTACAGTGCCTCTCTCACAAGGC | |
| | CCCAAACCAGTCACAATCAGTTTTGCCAATCACACTTCCTGCC | |
| | GGTGCATGTCTAAACTGGATGTTTACAGACAAGTTCATTCAA | |
| | TTATTAGACGT | |
| mouse VEGF- | GCACATTATAACACAGAGATCCTGAAAAGTATTGATAATGAG | 5 |
| C mature | TGGAGAAAGACTCAATGCATGCCACGTGAGGTGTGTATAGAT | |
| (dNdC) | GTGGGGAAGGAGTTTGGAGCAGCCACAAACACCTTCTTTAAA | |
| Cys152Ser | CCTCCATCTGTGTCCGTCTACAGATGTGGGGGTTGCTGCAACA | |
| mutation | GCGAGGGGCTGCAGTGCATGAACACCAGCACAGGTTACCTCA | |
| native | GCAAGACGTTGTTTGAAATTACAGTGCCTCTCTCACAAGGCC | |
| | CCAAACCAGTCACAATCAGTTTTGCCAATCACACTTCCTGCCG | |
| | GTGCATGTCTAAACTGGATGTTTACAGACAAGTTCATTCAATT | |
| | ATTAGACGT | |
| mouse VEGF- | GCACATTATAACACAGAGATCCTGAAAAGTATTGATAATGAG | 6 |
| C mature | TGGAGAAAGACTCAATGCATGCCACGTGAGGTGGCTATAGAT | |
| (dNdC) | GTGGGGAAGGAGTTTGGAGCAGCCACAAACACCTTCTTTAAA | |
| Cys133Ala | CCTCCATGTGTGTCCGTCTACAGATGTGGGGGTTGCTGCAAC | |
| mutation | AGCGAGGGGCTGCAGTGCATGAACACCAGCACAGGTTACCTC | |
| native | AGCAAGACGTTGTTTGAAATTACAGTGCCTCTCTCACAAGGC | |
| | CCCAAACCAGTCACAATCAGTTTTGCCAATCACACTTCCTGCC | |
| | GGTGCATGTCTAAACTGGATGTTTACAGACAAGTTCATTCAA | |
| | TTATTAGACGT | |
| mouse VEGF- | ATGCATCTGCTGTGTTTCCTGTCGCTCGCCTGCTCACTGCTGG | 7 |
| C full length | CGGCGGCACTTATCCCGTCCCCACGGGAGGCTCCTGCCACCG | |
| wt codon | TGGCCGCCTTCGAATCTGGGCTGGGCTTCAGCGAAGCCGAGC | |
| optimized 1 | CCGATGGCGGAGAGGTCAAGGCATTCGAAGGAAAGGACCTC | |
| (MS) | GAAGAACAGCTGAGATCCGTGTCCTCCGTGGACGAACTCATG | |
| (“SNI | TCCGTCCTGTACCCCGATTACTGGAAGATGTACAAATGCCAG | |
| VEGF-C FL | CTCCGGAAGGGCGGTTGGCAGCAGCCCACTCTGAACACTCGC | |
| WT” or “FL- | ACGGGAGATTCCGTGAAGTTTGCCGCCGCCCACTACAATACT | |
| WT”) | GAGATTCTCAAGTCCATCGACAACGAATGGAGGAAAACCCA | |
| | GTGTATGCCGCGCGAAGTCTGCATTGACGTGGGAAAGGAGTT | |
| | CGGCGCTGCCACCAACACCTTCTTTAAGCCTCCCTGCGTGTCG | |
| | GTGTATCGCTGCGGGGGATGCTGCAACAGCGAAGGCCTTCAG | |
| | TGCATGAACACCAGCACCGGATACCTCAGCAAGACTCTCTTC | |
| | GAAATCACTGTGCCGCTGTCACAAGGCCCGAAGCCTGTGACC | |
| | ATTTCCTTCGCCAACCACACCTCCTGTCGGTGCATGAGCAAGC | |
| | TGGATGTGTACAGACAGGTGCACTCCATCATCCGGAGATCGT | |
| | TGCCTGCCACCCTGCCGCAGTGCCAAGCGGCTAACAAGACCT | |
| | GTCCCACCAACTACGTGTGGAACAACTATATGTGTCGGTGCC | |
| | TGGCACAGCAGGACTTTATCTTCTACTCCAACGTGGAGGACG | |
| | ACTCGACTAACGGTTTCCACGACGTGTGCGGACCCAACAAGG | |
| | AGCTGGATGAGGATACTTGTCAGTGCGTGTGCAAGGGTGGCC | |
| | TGCGCCCGTCCTCCTGCGGACCACATAAGGAACTGGACAGGG | |
| | ACTCGTGCCAATGCGTCTGCAAGAACAAGCTGTTCCCTAACT | |
| | CCTGCGGGGCGAACCGCGAATTCGACGAGAACACCTGTCAGT | |
| | GTGTGTGCAAGCGGACTTGCCCGAGGAATCAGCCTCTTAACC | |
| | CCGGAAAATGCGCCTGCGAATGCACAGAGAACACCCAGAAG | |
| | TGCTTCTTGAAAGGGAAGAAGTTCCACCACCAAACCTGTTCA | |
| | TGCTACCGGCGCCCATGTGCCAACCGGCTGAAGCACTGCGAC | |
| | CCGGGATTGAGCTTCAGCGAGGAGGTCTGCAGATGCGTGCCG | |
| | TCATACTGGAAGCGACCTCATCTGAATTAGTGA | |
| mouse VEGF- | ATGCATCTGCTGTGTTTCCTGTCGCTCGCCTGCTCACTGCTGG | 8 |
| C full length | CGGCGGCACTTATCCCGTCCCCACGGGAGGCTCCTGCCACCG | |
| Cys152Ser | TGGCCGCCTTCGAATCTGGGCTGGGCTTCAGCGAAGCCGAGC | |
| mutation | CCGATGGCGGAGAGGTCAAGGCATTCGAAGGAAAGGACCTC | |
| codon | GAAGAACAGCTGAGATCCGTGTCCTCCGTGGACGAACTCATG | |
| optimized 1 | TCCGTCCTGTACCCCGATTACTGGAAGATGTACAAATGCCAG | |
| (MS) | CTCCGGAAGGGCGGTTGGCAGCAGCCCACTCTGAACACTCGC | |
| (“SN3 | ACGGGAGATTCCGTGAAGTTTGCCGCCGCCCACTACAATACT | |
| VEGF-C FL | GAGATTCTCAAGTCCATCGACAACGAATGGAGGAAAACCCA | |
| 152S” or “FL | GTGTATGCCGCGCGAAGTCTGCATTGACGTGGGAAAGGAGTT | |
| -152S”) | CGGCGCTGCCACCAACACCTTCTTTAAGCCTCCCTCCGTGTCG | |
| | GTGTATCGCTGCGGGGGATGCTGCAACAGCGAAGGCCTTCAG | |
| | TGCATGAACACCAGCACCGGATACCTCAGCAAGACTCTCTTC | |
| | GAAATCACTGTGCCGCTGTCACAAGGCCCGAAGCCTGTGACC | |
| | ATTTCCTTCGCCAACCACACCTCCTGTCGGTGCATGAGCAAGC | |
| | TGGATGTGTACAGACAGGTGCACTCCATCATCCGGAGATCGT | |
| | TGCCTGCCACCCTGCCGCAGTGCCAAGCGGCTAACAAGACCT | |
| | GTCCCACCAACTACGTGTGGAACAACTATATGTGTCGGTGCC | |
| | TGGCACAGCAGGACTTTATCTTCTACTCCAACGTGGAGGACG | |
| | ACTCGACTAACGGTTTCCACGACGTGTGCGGACCCAACAAGG | |
| | AGCTGGATGAGGATACTTGTCAGTGCGTGTGCAAGGGTGGCC | |
| | TGCGCCCGTCCTCCTGCGGACCACATAAGGAACTGGACAGGG | |
| | ACTCGTGCCAATGCGTCTGCAAGAACAAGCTGTTCCCTAACT | |
| | CCTGCGGGGCGAACCGCGAATTCGACGAGAACACCTGTCAGT | |
| | GTGTGTGCAAGCGGACTTGCCCGAGGAATCAGCCTCTTAACC | |
| | CCGGAAAATGCGCCTGCGAATGCACAGAGAACACCCAGAAG | |
| | TGCTTCTTGAAAGGGAAGAAGTTCCACCACCAAACCTGTTCA | |
| | TGCTACCGGCGCCCATGTGCCAACCGGCTGAAGCACTGCGAC | |
| | CCGGGATTGAGCTTCAGCGAGGAGGTCTGCAGATGCGTGCCG | |
| | TCATACTGGAAGCGACCTCATCTGAATTAGTGA | |
| mouse VEGF- | ATGCATCTGCTGTGTTTCCTGTCGCTCGCCTGCTCACTGCTGG | 9 |
| C full length | CGGCGGCACTTATCCCGTCCCCACGGGAGGCTCCTGCCACCG | |
| Cys133Ala | TGGCCGCCTTCGAATCTGGGCTGGGCTTCAGCGAAGCCGAGC | |
| mutation | CCGATGGCGGAGAGGTCAAGGCATTCGAAGGAAAGGACCTC | |
| codon | GAAGAACAGCTGAGATCCGTGTCCTCCGTGGACGAACTCATG | |
| optimized 1 | TCCGTCCTGTACCCCGATTACTGGAAGATGTACAAATGCCAG | |
| (MS) | CTCCGGAAGGGCGGTTGGCAGCAGCCCACTCTGAACACTCGC | |
| (“SN2 VEGF- | ACGGGAGATTCCGTGAAGTTTGCCGCCGCCCACTACAATACT | |
| CFL 133A” | GAGATTCTCAAGTCCATCGACAACGAATGGAGGAAAACCCA | |
| or “FL- | GTGTATGCCGCGCGAAGTCGCCATTGACGTGGGAAAGGAGTT | |
| 133A”) | CGGCGCTGCCACCAACACCTTCTTTAAGCCTCCCTGCGTGTCG | |
| | GTGTATCGCTGCGGGGGATGCTGCAACAGCGAAGGCCTTCAG | |
| | TGCATGAACACCAGCACCGGATACCTCAGCAAGACTCTCTTC | |
| | GAAATCACTGTGCCGCTGTCACAAGGCCCGAAGCCTGTGACC | |
| | ATTTCCTTCGCCAACCACACCTCCTGTCGGTGCATGAGCAAGC | |
| | TGGATGTGTACAGACAGGTGCACTCCATCATCCGGAGATCGT | |
| | TGCCTGCCACCCTGCCGCAGTGCCAAGCGGCTAACAAGACCT | |
| | GTCCCACCAACTACGTGTGGAACAACTATATGTGTCGGTGCC | |
| | TGGCACAGCAGGACTTTATCTTCTACTCCAACGTGGAGGACG | |
| | ACTCGACTAACGGTTTCCACGACGTGTGCGGACCCAACAAGG | |
| | AGCTGGATGAGGATACTTGTCAGTGCGTGTGCAAGGGTGGCC | |
| | TGCGCCCGTCCTCCTGCGGACCACATAAGGAACTGGACAGGG | |
| | ACTCGTGCCAATGCGTCTGCAAGAACAAGCTGTTCCCTAACT | |
| | CCTGCGGGGCGAACCGCGAATTCGACGAGAACACCTGTCAGT | |
| | GTGTGTGCAAGCGGACTTGCCCGAGGAATCAGCCTCTTAACC | |
| | CCGGAAAATGCGCCTGCGAATGCACAGAGAACACCCAGAAG | |
| | TGCTTCTTGAAAGGGAAGAAGTTCCACCACCAAACCTGTTCA | |
| | TGCTACCGGCGCCCATGTGCCAACCGGCTGAAGCACTGCGAC | |
| | CCGGGATTGAGCTTCAGCGAGGAGGTCTGCAGATGCGTGCCG | |
| | TCATACTGGAAGCGACCTCATCTGAATTAGTGA | |
| mouse VEGF- | GCCCACTACAATACTGAGATTCTCAAGTCCATCGACAACGAA | 10 |
| C mature | TGGAGGAAAACCCAGTGTATGCCGCGCGAAGTCTGCATTGAC | |
| (dNdC) wt | GTGGGAAAGGAGTTCGGCGCTGCCACCAACACCTTCTTTAAG | |
| codon | CCTCCCTGCGTGTCGGTGTATCGCTGCGGGGGATGCTGCAAC | |
| optimized 1 | AGCGAAGGCCTTCAGTGCATGAACACCAGCACCGGATACCTC | |
| (MS) | AGCAAGACTCTCTTCGAAATCACTGTGCCGCTGTCACAAGGC | |
| (SN4 | CCGAAGCCTGTGACCATTTCCTTCGCCAACCACACCTCCTGTC | |
| VEGF-C | GGTGCATGAGCAAGCTGGATGTGTACAGACAGGTGCACTCCA | |
| DNDC WT” | TCATCCGGAGA | |
| or “dNdC- | | |
| WT”) | | |
| mouse VEGF- | GCCCACTACAATACTGAGATTCTCAAGTCCATCGACAACGAA | 11 |
| C mature | TGGAGGAAAACCCAGTGTATGCCGCGCGAAGTCTGCATTGAC | |
| (dNdC) | GTGGGAAAGGAGTTCGGCGCTGCCACCAACACCTTCTTTAAG | |
| Cys152Ser | CCTCCCTCCGTGTCGGTGTATCGCTGCGGGGGATGCTGCAAC | |
| mutation | AGCGAAGGCCTTCAGTGCATGAACACCAGCACCGGATACCTC | |
| codon | AGCAAGACTCTCTTCGAAATCACTGTGCCGCTGTCACAAGGC | |
| optimized 1 | CCGAAGCCTGTGACCATTTCCTTCGCCAACCACACCTCCTGTC | |
| (MS) | GGTGCATGAGCAAGCTGGATGTGTACAGACAGGTGCACTCCA | |
| (SN6 VEGF- | TCATCCGGAGA | |
| CDNDC | | |
| 152S” or | | |
| “dNdC- | | |
| 152S”) | | |
| mouse VEGF- | GCCCACTACAATACTGAGATTCTCAAGTCCATCGACAACGAA | 12 |
| C mature | TGGAGGAAAACCCAGTGTATGCCGCGCGAAGTCGCCATTGAC | |
| (dNdC) | GTGGGAAAGGAGTTCGGCGCTGCCACCAACACCTTCTTTAAG | |
| Cys133Ala | CCTCCCTGCGTGTCGGTGTATCGCTGCGGGGGATGCTGCAAC | |
| mutation | AGCGAAGGCCTTCAGTGCATGAACACCAGCACCGGATACCTC | |
| codon | AGCAAGACTCTCTTCGAAATCACTGTGCCGCTGTCACAAGGC | |
| optimized 1 | CCGAAGCCTGTGACCATTTCCTTCGCCAACCACACCTCCTGTC | |
| (MS) | GGTGCATGAGCAAGCTGGATGTGTACAGACAGGTGCACTCCA | |
| (“SN5 VEGF- | TCATCCGGAGA | |
| CDNDC | | |
| 133A” or | | |
| “dNdC- | | |
| 133A”) | | |
| mouse VEGF- | ATGCACCTGCTGTGCTTCCTGAGCCTGGCCTGCAGCCTGCTGG | 13 |
| C full length | CCGCCGCCCTGATCCCCAGCCCCAGAGAGGCCCCCGCCACCG | |
| wt codon | TGGCCGCCTTCGAGAGCGGCCTGGGCTTCAGCGAGGCCGAGC | |
| optimized 2 | CCGACGGCGGCGAGGTGAAGGCCTTCGAGGGCAAGGACCTG | |
| (AGS) | GAGGAGCAGCTGAGAAGCGTGAGCAGCGTGGACGAGCTGAT | |
| | GAGCGTGCTGTACCCCGACTACTGGAAGATGTACAAGTGCCA | |
| | GCTGAGAAAGGGCGGCTGGCAGCAGCCCACCCTGAACACCA | |
| | GAACCGGCGACAGCGTGAAGTTCGCCGCCGCCCACTACAACA | |
| | CCGAGATCCTGAAGAGCATCGACAACGAGTGGAGAAAGACC | |
| | CAGTGCATGCCCAGAGAGGTGTGCATCGACGTGGGCAAGGA | |
| | GTTCGGCGCCGCCACCAACACCTTCTTCAAGCCCCCCTGCGTG | |
| | AGCGTGTACAGATGCGGCGGCTGCTGCAACAGCGAGGGCCTG | |
| | CAGTGCATGAACACCAGCACCGGCTACCTGAGCAAGACCCTG | |
| | TTCGAGATCACCGTGCCCCTGAGCCAGGGCCCCAAGCCCGTG | |
| | ACCATCAGCTTCGCCAACCACACCAGCTGCAGATGCATGAGC | |
| | AAGCTGGACGTGTACAGACAGGTGCACAGCATCATCAGAAG | |
| | AAGCCTGCCCGCCACCCTGCCCCAGTGCCAGGCCGCCAACAA | |
| | GACCTGCCCCACCAACTACGTGTGGAACAACTACATGTGCAG | |
| | ATGCCTGGCCCAGCAGGACTTCATCTTCTACAGCAACGTGGA | |
| | GGACGACAGCACCAACGGCTTCCACGACGTGTGCGGCCCCAA | |
| | CAAGGAGCTGGACGAGGACACCTGCCAGTGCGTGTGCAAGG | |
| | GCGGCCTGAGACCCAGCAGCTGCGGCCCCCACAAGGAGCTG | |
| | GACAGAGACAGCTGCCAGTGCGTGTGCAAGAACAAGCTGTTC | |
| | CCCAACAGCTGCGGCGCCAACAGAGAGTTCGACGAGAACAC | |
| | CTGCCAGTGCGTGTGCAAGAGAACCTGCCCCAGAAACCAGCC | |
| | CCTGAACCCCGGCAAGTGCGCCTGCGAGTGCACCGAGAACAC | |
| | CCAGAAGTGCTTCCTGAAGGGCAAGAAGTTCCACCACCAGAC | |
| | CTGCAGCTGCTACAGAAGACCCTGCGCCAACAGACTGAAGCA | |
| | CTGCGACCCCGGCCTGAGCTTCAGCGAGGAGGTGTGCAGATG | |
| | CGTGCCCAGCTACTGGAAGAGACCCCACCTGAAC | |
| mouse VEGF- | ATGCACCTGCTGTGCTTCCTGAGCCTGGCCTGCAGCCTGCTGG | 14 |
| C full length | CCGCCGCCCTGATCCCCAGCCCCAGAGAGGCCCCCGCCACCG | |
| Cys152Ser | TGGCCGCCTTCGAGAGCGGCCTGGGCTTCAGCGAGGCCGAGC | |
| mutation | CCGACGGCGGCGAGGTGAAGGCCTTCGAGGGCAAGGACCTG | |
| codon | GAGGAGCAGCTGAGAAGCGTGAGCAGCGTGGACGAGCTGAT | |
| optimized 2 | GAGCGTGCTGTACCCCGACTACTGGAAGATGTACAAGTGCCA | |
| (AGS) | GCTGAGAAAGGGCGGCTGGCAGCAGCCCACCCTGAACACCA | |
| | GAACCGGCGACAGCGTGAAGTTCGCCGCCGCCCACTACAACA | |
| | CCGAGATCCTGAAGAGCATCGACAACGAGTGGAGAAAGACC | |
| | CAGTGCATGCCCAGAGAGGTGTGCATCGACGTGGGCAAGGA | |
| | GTTCGGCGCCGCCACCAACACCTTCTTCAAGCCCCCCTCCGTG | |
| | AGCGTGTACAGATGCGGCGGCTGCTGCAACAGCGAGGGCCTG | |
| | CAGTGCATGAACACCAGCACCGGCTACCTGAGCAAGACCCTG | |
| | TTCGAGATCACCGTGCCCCTGAGCCAGGGCCCCAAGCCCGTG | |
| | ACCATCAGCTTCGCCAACCACACCAGCTGCAGATGCATGAGC | |
| | AAGCTGGACGTGTACAGACAGGTGCACAGCATCATCAGAAG | |
| | AAGCCTGCCCGCCACCCTGCCCCAGTGCCAGGCCGCCAACAA | |
| | GACCTGCCCCACCAACTACGTGTGGAACAACTACATGTGCAG | |
| | ATGCCTGGCCCAGCAGGACTTCATCTTCTACAGCAACGTGGA | |
| | GGACGACAGCACCAACGGCTTCCACGACGTGTGCGGCCCCAA | |
| | CAAGGAGCTGGACGAGGACACCTGCCAGTGCGTGTGCAAGG | |
| | GCGGCCTGAGACCCAGCAGCTGCGGCCCCCACAAGGAGCTG | |
| | GACAGAGACAGCTGCCAGTGCGTGTGCAAGAACAAGCTGTTC | |
| | CCCAACAGCTGCGGCGCCAACAGAGAGTTCGACGAGAACAC | |
| | CTGCCAGTGCGTGTGCAAGAGAACCTGCCCCAGAAACCAGCC | |
| | CCTGAACCCCGGCAAGTGCGCCTGCGAGTGCACCGAGAACAC | |
| | CCAGAAGTGCTTCCTGAAGGGCAAGAAGTTCCACCACCAGAC | |
| | CTGCAGCTGCTACAGAAGACCCTGCGCCAACAGACTGAAGCA | |
| | CTGCGACCCCGGCCTGAGCTTCAGCGAGGAGGTGTGCAGATG | |
| | CGTGCCCAGCTACTGGAAGAGACCCCACCTGAAC | |
| mouse VEGF- | ATGCACCTGCTGTGCTTCCTGAGCCTGGCCTGCAGCCTGCTGG | 15 |
| C full length | CCGCCGCCCTGATCCCCAGCCCCAGAGAGGCCCCCGCCACCG | |
| Cys133Ala | TGGCCGCCTTCGAGAGCGGCCTGGGCTTCAGCGAGGCCGAGC | |
| mutation | CCGACGGCGGCGAGGTGAAGGCCTTCGAGGGCAAGGACCTG | |
| codon | GAGGAGCAGCTGAGAAGCGTGAGCAGCGTGGACGAGCTGAT | |
| optimized 2 | GAGCGTGCTGTACCCCGACTACTGGAAGATGTACAAGTGCCA | |
| (AGS) | GCTGAGAAAGGGCGGCTGGCAGCAGCCCACCCTGAACACCA | |
| | GAACCGGCGACAGCGTGAAGTTCGCCGCCGCCCACTACAACA | |
| | CCGAGATCCTGAAGAGCATCGACAACGAGTGGAGAAAGACC | |
| | CAGTGCATGCCCAGAGAGGTGGCCATCGACGTGGGCAAGGA | |
| | GTTCGGCGCCGCCACCAACACCTTCTTCAAGCCCCCCTGCGTG | |
| | AGCGTGTACAGATGCGGCGGCTGCTGCAACAGCGAGGGCCTG | |
| | CAGTGCATGAACACCAGCACCGGCTACCTGAGCAAGACCCTG | |
| | TTCGAGATCACCGTGCCCCTGAGCCAGGGCCCCAAGCCCGTG | |
| | ACCATCAGCTTCGCCAACCACACCAGCTGCAGATGCATGAGC | |
| | AAGCTGGACGTGTACAGACAGGTGCACAGCATCATCAGAAG | |
| | AAGCCTGCCCGCCACCCTGCCCCAGTGCCAGGCCGCCAACAA | |
| | GACCTGCCCCACCAACTACGTGTGGAACAACTACATGTGCAG | |
| | ATGCCTGGCCCAGCAGGACTTCATCTTCTACAGCAACGTGGA | |
| | GGACGACAGCACCAACGGCTTCCACGACGTGTGCGGCCCCAA | |
| | CAAGGAGCTGGACGAGGACACCTGCCAGTGCGTGTGCAAGG | |
| | GCGGCCTGAGACCCAGCAGCTGCGGCCCCCACAAGGAGCTG | |
| | GACAGAGACAGCTGCCAGTGCGTGTGCAAGAACAAGCTGTTC | |
| | CCCAACAGCTGCGGCGCCAACAGAGAGTTCGACGAGAACAC | |
| | CTGCCAGTGCGTGTGCAAGAGAACCTGCCCCAGAAACCAGCC | |
| | CCTGAACCCCGGCAAGTGCGCCTGCGAGTGCACCGAGAACAC | |
| | CCAGAAGTGCTTCCTGAAGGGCAAGAAGTTCCACCACCAGAC | |
| | CTGCAGCTGCTACAGAAGACCCTGCGCCAACAGACTGAAGCA | |
| | CTGCGACCCCGGCCTGAGCTTCAGCGAGGAGGTGTGCAGATG | |
| | CGTGCCCAGCTACTGGAAGAGACCCCACCTGAAC | |
| mouse VEGF- | GCCCACTACAACACCGAGATCCTGAAGAGCATCGACAACGA | 16 |
| C mature | GTGGAGAAAGACCCAGTGCATGCCCAGAGAGGTGTGCATCG | |
| (dNdC) wt | ACGTGGGCAAGGAGTTCGGCGCCGCCACCAACACCTTCTTCA | |
| codon | AGCCCCCCTGCGTGAGCGTGTACAGATGCGGCGGCTGCTGCA | |
| optimized 2 | ACAGCGAGGGCCTGCAGTGCATGAACACCAGCACCGGCTACC | |
| (AGS) | TGAGCAAGACCCTGTTCGAGATCACCGTGCCCCTGAGCCAGG | |
| | GCCCCAAGCCCGTGACCATCAGCTTCGCCAACCACACCAGCT | |
| | GCAGATGCATGAGCAAGCTGGACGTGTACAGACAGGTGCAC | |
| | AGCATCATCAGAAGA | |
| mouse VEGF- | GCCCACTACAACACCGAGATCCTGAAGAGCATCGACAACGA | 17 |
| C mature | GTGGAGAAAGACCCAGTGCATGCCCAGAGAGGTGTGCATCG | |
| (dNdC) | ACGTGGGCAAGGAGTTCGGCGCCGCCACCAACACCTTCTTCA | |
| Cys152Ser | AGCCCCCCTCCGTGAGCGTGTACAGATGCGGCGGCTGCTGCA | |
| mutation | ACAGCGAGGGCCTGCAGTGCATGAACACCAGCACCGGCTACC | |
| codon | TGAGCAAGACCCTGTTCGAGATCACCGTGCCCCTGAGCCAGG | |
| optimized 2 | GCCCCAAGCCCGTGACCATCAGCTTCGCCAACCACACCAGCT | |
| (AGS) | GCAGATGCATGAGCAAGCTGGACGTGTACAGACAGGTGCAC | |
| | AGCATCATCAGAAGA | |
| mouse VEGF- | GCCCACTACAACACCGAGATCCTGAAGAGCATCGACAACGA | 18 |
| C mature | GTGGAGAAAGACCCAGTGCATGCCCAGAGAGGTGGCCATCG | |
| (dNdC) | ACGTGGGCAAGGAGTTCGGCGCCGCCACCAACACCTTCTTCA | |
| Cys133Ala | AGCCCCCCTGCGTGAGCGTGTACAGATGCGGCGGCTGCTGCA | |
| mutation | ACAGCGAGGGCCTGCAGTGCATGAACACCAGCACCGGCTACC | |
| codon | TGAGCAAGACCCTGTTCGAGATCACCGTGCCCCTGAGCCAGG | |
| optimized 2 | GCCCCAAGCCCGTGACCATCAGCTTCGCCAACCACACCAGCT | |
| (AGS) | GCAGATGCATGAGCAAGCTGGACGTGTACAGACAGGTGCAC | |
| | AGCATCATCAGAAGA | |
| mouse VEGF- | MHLLCFLSLACSLLAAALIPSPREAPATVAAFESGLGFSEAEPDG | 19 |
| C full length | GEVKAFEGKDLEEQLRSVSSVDELMSVLYPDYWKMYKCQLRK | |
| wt | GGWQQPTLNTRTGDSVKFAAAHYNTEILKSIDNEWRKTQCMPR | |
| | EVCIDVGKEFGAATNTFFKPPCVSVYRCGGCCNSEGLQCMNTST | |
| | GYLSKTLFEITVPLSQGPKPVTISFANHTSCRCMSKLDVYRQVHS | |
| | IIRRSLPATLPQCQAANKTCPTNYVWNNYMCRCLAQQDFIFYSN | |
| | VEDDSTNGFHDVCGPNKELDEDTCQCVCKGGLRPSSCGPHKEL | |
| | DRDSCQCVCKNKLFPNSCGANREFDENTCQCVCKRTCPRNQPL | |
| | NPGKCACECTENTQKCFLKGKKFHHQTCSCYRRPCANRLKHCD | |
| | PGLSFSEEVCRCVPSYWKRPHLN | |
| mouse VEGF- | MHLLCFLSLACSLLAAALIPSPREAPATVAAFESGLGFSEAEPDG | 20 |
| C full length | GEVKAFEGKDLEEQLRSVSSVDELMSVLYPDYWKMYKCQLRK | |
| Cys152Ser | GGWQQPTLNTRTGDSVKFAAAHYNTEILKSIDNEWRKTQCMPR | |
| mutation | EVCIDVGKEFGAATNTFFKPPSVSVYRCGGCCNSEGLQCMNTST | |
| | GYLSKTLFEITVPLSQGPKPVTISFANHTSCRCMSKLDVYRQVHS | |
| | IIRRSLPATLPQCQAANKTCPTNYVWNNYMCRCLAQQDFIFYSN | |
| | VEDDSTNGFHDVCGPNKELDEDTCQCVCKGGLRPSSCGPHKEL | |
| | DRDSCQCVCKNKLFPNSCGANREFDENTCQCVCKRTCPRNQPL | |
| | NPGKCACECTENTQKCFLKGKKFHHQTCSCYRRPCANRLKHCD | |
| | PGLSFSEEVCRCVPSYWKRPHLN | |
| mouse VEGF- | MHLLCFLSLACSLLAAALIPSPREAPATVAAFESGLGFSEAEPDG | 21 |
| C full length | GEVKAFEGKDLEEQLRSVSSVDELMSVLYPDYWKMYKCQLRK | |
| Cys133Ala | GGWQQPTLNTRTGDSVKFAAAHYNTEILKSIDNEWRKTQCMPR | |
| mutation | EVAIDVGKEFGAATNTFFKPPCVSVYRCGGCCNSEGLQCMNTST | |
| | GYLSKTLFEITVPLSQGPKPVTISFANHTSCRCMSKLDVYRQVHS | |
| | IIRRSLPATLPQCQAANKTCPTNYVWNNYMCRCLAQQDFIFYSN | |
| | VEDDSTNGFHDVCGPNKELDEDTCQCVCKGGLRPSSCGPHKEL | |
| | DRDSCQCVCKNKLFPNSCGANREFDENTCQCVCKRTCPRNQPL | |
| | NPGKCACECTENTQKCFLKGKKFHHQTCSCYRRPCANRLKHCD | |
| | PGLSFSEEVCRCVPSYWKRPHLN | |
| mouse VEGF- | AHYNTEILKSIDNEWRKTQCMPREVCIDVGKEFGAATNTFFKPP | 22 |
| C mature | CVSVYRCGGCCNSEGLQCMNTSTGYLSKTLFEITVPLSQGPKPV | |
| (dNdC) wt | TISFANHTSCRCMSKLDVYRQVHSIIRR | |
| mouse VEGF- | AHYNTEILKSIDNEWRKTQCMPREVCIDVGKEFGAATNTFFKPP | 23 |
| C mature | SVSVYRCGGCCNSEGLQCMNTSTGYLSKTLFEITVPLSQGPKPV | |
| (dNdC) | TISFANHTSCRCMSKLDVYRQVHSIIRR | |
| Cys152Ser | | |
| mouse VEGF- | AHYNTEILKSIDNEWRKTQCMPREVAIDVGKEFGAATNTFFKPP | 24 |
| C mature | CVSVYRCGGCCNSEGLQCMNTSTGYLSKTLFEITVPLSQGPKPV | |
| (dNdC) | TISFANHTSCRCMSKLDVYRQVHSIIRR | |
| Cys133Ala | | |
| Signal peptide | ATGCGGGTGCCCGCCCAGCTGCTGGGCCTGCTCCTGCTCTGG | 25 |
| IgG light | CTGCCAGGCGCTAGATGT | |
| chain | | |
| Signal peptide | MRVPAQLLGLLLLWLPGARC | 26 |
| IgG light | | |
| chain | | |
| Signal peptide | ATGGGCGTGAAGGTGCTGTTCGCCCTGATCTGCATCGCCGTG | 27 |
| Gaussia | GCCGAGGCC | |
| luciferase | | |
| Signal peptide | MGVKVLFALICIAVAEA | 28 |
| Gaussia | | |
| luciferase | | |
| human VEGF- | ATGCACTTGCTGGGCTTCTTCTCTGTGGCGTGTTCTCTGCTCG | 29 |
| C full length | CCGCTGCGCTGCTCCCGGGTCCTCGCGAGGCGCCCGCCGCCG | |
| wt native | CCGCCGCCTTCGAGTCCGGACTCGACCTCTCGGACGCGGAGC | |
| | CCGACGCGGGCGAGGCCACGGCTTATGCAAGCAAAGATCTG | |
| | GAGGAGCAGTTACGGTCTGTGTCCAGTGTAGATGAACTCATG | |
| | ACTGTACTCTACCCAGAATATTGGAAAATGTACAAGTGTCAG | |
| | CTAAGGAAAGGAGGCTGGCAACATAACAGAGAACAGGCCAA | |
| | CCTCAACTCAAGGACAGAAGAGACTATAAAATTTGCTGCAGC | |
| | ACATTATAATACAGAGATCTTGAAAAGTATTGATAATGAGTG | |
| | GAGAAAGACTCAATGCATGCCACGGGAGGTGTGTATAGATGT | |
| | GGGGAAGGAGTTTGGAGTCGCGACAAACACCTTCTTTAAACC | |
| | TCCATGTGTGTCCGTCTACAGATGTGGGGGTTGCTGCAATAGT | |
| | GAGGGGCTGCAGTGCATGAACACCAGCACGAGCTACCTCAGC | |
| | AAGACGTTATTTGAAATTACAGTGCCTCTCTCTCAAGGCCCCA | |
| | AACCAGTAACAATCAGTTTTGCCAATCACACTTCCTGCCGAT | |
| | GCATGTCTAAACTGGATGTTTACAGACAAGTTCATTCCATTAT | |
| | TAGACGTTCCCTGCCAGCAACACTACCACAGTGTCAGGCAGC | |
| | GAACAAGACCTGCCCCACCAATTACATGTGGAATAATCACAT | |
| | CTGCAGATGCCTGGCTCAGGAAGATTTTATGTTTTCCTCGGAT | |
| | GCTGGAGATGACTCAACAGATGGATTCCATGACATCTGTGGA | |
| | CCAAACAAGGAGCTGGATGAAGAGACCTGTCAGTGTGTCTGC | |
| | AGAGCGGGGCTTCGGCCTGCCAGCTGTGGACCCCACAAAGAA | |
| | CTAGACAGAAACTCATGCCAGTGTGTCTGTAAAAACAAACTC | |
| | TTCCCCAGCCAATGTGGGGCCAACCGAGAATTTGATGAAAAC | |
| | ACATGCCAGTGTGTATGTAAAAGAACCTGCCCCAGAAATCAA | |
| | CCCCTAAATCCTGGAAAATGTGCCTGTGAATGTACAGAAAGT | |
| | CCACAGAAATGCTTGTTAAAAGGAAAGAAGTTCCACCACCAA | |
| | ACATGCAGCTGTTACAGACGGCCATGTACGAACCGCCAGAAG | |
| | GCTTGTGAGCCAGGATTTTCATATAGTGAAGAAGTGTGTCGT | |
| | TGTGTCCCTTCATATTGGAAAAGACCACAAATGAGCTAA | |
| human VEGF- | ATGCACTTGCTGGGCTTCTTCTCTGTGGCGTGTTCTCTGCTCG | 30 |
| C full length | CCGCTGCGCTGCTCCCGGGTCCTCGCGAGGCGCCCGCCGCCG | |
| Cys156Ser | CCGCCGCCTTCGAGTCCGGACTCGACCTCTCGGACGCGGAGC | |
| mutation | CCGACGCGGGCGAGGCCACGGCTTATGCAAGCAAAGATCTG | |
| native | GAGGAGCAGTTACGGTCTGTGTCCAGTGTAGATGAACTCATG | |
| | ACTGTACTCTACCCAGAATATTGGAAAATGTACAAGTGTCAG | |
| | CTAAGGAAAGGAGGCTGGCAACATAACAGAGAACAGGCCAA | |
| | CCTCAACTCAAGGACAGAAGAGACTATAAAATTTGCTGCAGC | |
| | ACATTATAATACAGAGATCTTGAAAAGTATTGATAATGAGTG | |
| | GAGAAAGACTCAATGCATGCCACGGGAGGTGTGTATAGATGT | |
| | GGGGAAGGAGTTTGGAGTCGCGACAAACACCTTCTTTAAACC | |
| | TCCATCTGTGTCCGTCTACAGATGTGGGGGTTGCTGCAATAGT | |
| | GAGGGGCTGCAGTGCATGAACACCAGCACGAGCTACCTCAGC | |
| | AAGACGTTATTTGAAATTACAGTGCCTCTCTCTCAAGGCCCCA | |
| | AACCAGTAACAATCAGTTTTGCCAATCACACTTCCTGCCGAT | |
| | GCATGTCTAAACTGGATGTTTACAGACAAGTTCATTCCATTAT | |
| | TAGACGTTCCCTGCCAGCAACACTACCACAGTGTCAGGCAGC | |
| | GAACAAGACCTGCCCCACCAATTACATGTGGAATAATCACAT | |
| | CTGCAGATGCCTGGCTCAGGAAGATTTTATGTTTTCCTCGGAT | |
| | GCTGGAGATGACTCAACAGATGGATTCCATGACATCTGTGGA | |
| | CCAAACAAGGAGCTGGATGAAGAGACCTGTCAGTGTGTCTGC | |
| | AGAGCGGGGCTTCGGCCTGCCAGCTGTGGACCCCACAAAGAA | |
| | CTAGACAGAAACTCATGCCAGTGTGTCTGTAAAAACAAACTC | |
| | TTCCCCAGCCAATGTGGGGCCAACCGAGAATTTGATGAAAAC | |
| | ACATGCCAGTGTGTATGTAAAAGAACCTGCCCCAGAAATCAA | |
| | CCCCTAAATCCTGGAAAATGTGCCTGTGAATGTACAGAAAGT | |
| | CCACAGAAATGCTTGTTAAAAGGAAAGAAGTTCCACCACCAA | |
| | ACATGCAGCTGTTACAGACGGCCATGTACGAACCGCCAGAAG | |
| | GCTTGTGAGCCAGGATTTTCATATAGTGAAGAAGTGTGTCGT | |
| | TGTGTCCCTTCATATTGGAAAAGACCACAAATGAGCTAA | |
| human VEGF- | ATGCACTTGCTGGGCTTCTTCTCTGTGGCGTGTTCTCTGCTCG | 31 |
| C full length | CCGCTGCGCTGCTCCCGGGTCCTCGCGAGGCGCCCGCCGCCG | |
| Cys137A1a | CCGCCGCCTTCGAGTCCGGACTCGACCTCTCGGACGCGGAGC | |
| mutation | CCGACGCGGGCGAGGCCACGGCTTATGCAAGCAAAGATCTG | |
| native | GAGGAGCAGTTACGGTCTGTGTCCAGTGTAGATGAACTCATG | |
| | ACTGTACTCTACCCAGAATATTGGAAAATGTACAAGTGTCAG | |
| | CTAAGGAAAGGAGGCTGGCAACATAACAGAGAACAGGCCAA | |
| | CCTCAACTCAAGGACAGAAGAGACTATAAAATTTGCTGCAGC | |
| | ACATTATAATACAGAGATCTTGAAAAGTATTGATAATGAGTG | |
| | GAGAAAGACTCAATGCATGCCACGGGAGGTGGCTATAGATGT | |
| | GGGGAAGGAGTTTGGAGTCGCGACAAACACCTTCTTTAAACC | |
| | TCCATGTGTGTCCGTCTACAGATGTGGGGGTTGCTGCAATAGT | |
| | GAGGGGCTGCAGTGCATGAACACCAGCACGAGCTACCTCAGC | |
| | AAGACGTTATTTGAAATTACAGTGCCTCTCTCTCAAGGCCCCA | |
| | AACCAGTAACAATCAGTTTTGCCAATCACACTTCCTGCCGAT | |
| | GCATGTCTAAACTGGATGTTTACAGACAAGTTCATTCCATTAT | |
| | TAGACGTTCCCTGCCAGCAACACTACCACAGTGTCAGGCAGC | |
| | GAACAAGACCTGCCCCACCAATTACATGTGGAATAATCACAT | |
| | CTGCAGATGCCTGGCTCAGGAAGATTTTATGTTTTCCTCGGAT | |
| | GCTGGAGATGACTCAACAGATGGATTCCATGACATCTGTGGA | |
| | CCAAACAAGGAGCTGGATGAAGAGACCTGTCAGTGTGTCTGC | |
| | AGAGCGGGGCTTCGGCCTGCCAGCTGTGGACCCCACAAAGAA | |
| | CTAGACAGAAACTCATGCCAGTGTGTCTGTAAAAACAAACTC | |
| | TTCCCCAGCCAATGTGGGGCCAACCGAGAATTTGATGAAAAC | |
| | ACATGCCAGTGTGTATGTAAAAGAACCTGCCCCAGAAATCAA | |
| | CCCCTAAATCCTGGAAAATGTGCCTGTGAATGTACAGAAAGT | |
| | CCACAGAAATGCTTGTTAAAAGGAAAGAAGTTCCACCACCAA | |
| | ACATGCAGCTGTTACAGACGGCCATGTACGAACCGCCAGAAG | |
| | GCTTGTGAGCCAGGATTTTCATATAGTGAAGAAGTGTGTCGT | |
| | TGTGTCCCTTCATATTGGAAAAGACCACAAATGAGCTAA | |
| human VEGF- | GCACATTATAATACAGAGATCTTGAAAAGTATTGATAATGAG | 32 |
| C mature | TGGAGAAAGACTCAATGCATGCCACGGGAGGTGTGTATAGAT | |
| (dNdC) wt | GTGGGGAAGGAGTTTGGAGTCGCGACAAACACCTTCTTTAAA | |
| native | CCTCCATGTGTGTCCGTCTACAGATGTGGGGGTTGCTGCAATA | |
| | GTGAGGGGCTGCAGTGCATGAACACCAGCACGAGCTACCTCA | |
| | GCAAGACGTTATTTGAAATTACAGTGCCTCTCTCTCAAGGCCC | |
| | CAAACCAGTAACAATCAGTTTTGCCAATCACACTTCCTGCCG | |
| | ATGCATGTCTAAACTGGATGTTTACAGACAAGTTCATTCCATT | |
| | ATTAGACGT | |
| human VEGF- | GCACATTATAATACAGAGATCTTGAAAAGTATTGATAATGAG | 33 |
| C mature | TGGAGAAAGACTCAATGCATGCCACGGGAGGTGTGTATAGAT | |
| (dNdC) | GTGGGGAAGGAGTTTGGAGTCGCGACAAACACCTTCTTTAAA | |
| Cys156Ser | CCTCCATCTGTGTCCGTCTACAGATGTGGGGGTTGCTGCAATA | |
| mutation | GTGAGGGGCTGCAGTGCATGAACACCAGCACGAGCTACCTCA | |
| native | GCAAGACGTTATTTGAAATTACAGTGCCTCTCTCTCAAGGCCC | |
| | CAAACCAGTAACAATCAGTTTTGCCAATCACACTTCCTGCCG | |
| | ATGCATGTCTAAACTGGATGTTTACAGACAAGTTCATTCCATT | |
| | ATTAGACGT | |
| human VEGF- | GCACATTATAATACAGAGATCTTGAAAAGTATTGATAATGAG | 34 |
| C mature | TGGAGAAAGACTCAATGCATGCCACGGGAGGTGGCTATAGAT | |
| (dNdC) | GTGGGGAAGGAGTTTGGAGTCGCGACAAACACCTTCTTTAAA | |
| Cys137A1a | CCTCCATGTGTGTCCGTCTACAGATGTGGGGGTTGCTGCAATA | |
| mutation | GTGAGGGGCTGCAGTGCATGAACACCAGCACGAGCTACCTCA | |
| native | GCAAGACGTTATTTGAAATTACAGTGCCTCTCTCTCAAGGCCC | |
| | CAAACCAGTAACAATCAGTTTTGCCAATCACACTTCCTGCCG | |
| | ATGCATGTCTAAACTGGATGTTTACAGACAAGTTCATTCCATT | |
| | ATTAGACGT | |
| human VEGF- | ATGCACCTGCTGGGCTTCTTCAGCGTGGCCTGCAGCCTGCTGG | 35 |
| C full length | CCGCCGCCCTGCTGCCCGGCCCCAGAGAGGCCCCCGCCGCCG | |
| wt codon | CCGCCGCCTTCGAGAGCGGCCTGGACCTGAGCGACGCCGAGC | |
| optimized 2 | CCGACGCCGGCGAGGCCACCGCCTACGCCAGCAAGGACCTG | |
| (AGS) | GAGGAGCAGCTGAGAAGCGTGAGCAGCGTGGACGAGCTGAT | |
| | GACCGTGCTGTACCCCGAGTACTGGAAGATGTACAAGTGCCA | |
| | GCTGAGAAAGGGCGGCTGGCAGCACAACAGAGAGCAGGCCA | |
| | ACCTGAACAGCAGAACCGAGGAGACCATCAAGTTCGCCGCC | |
| | GCCCACTACAACACCGAGATCCTGAAGAGCATCGACAACGA | |
| | GTGGAGAAAGACCCAGTGCATGCCCAGAGAGGTGTGCATCG | |
| | ACGTGGGCAAGGAGTTCGGCGTGGCCACCAACACCTTCTTCA | |
| | AGCCCCCCTGCGTGAGCGTGTACAGATGCGGCGGCTGCTGCA | |
| | ACAGCGAGGGCCTGCAGTGCATGAACACCAGCACCAGCTACC | |
| | TGAGCAAGACCCTGTTCGAGATCACCGTGCCCCTGAGCCAGG | |
| | GCCCCAAGCCCGTGACCATCAGCTTCGCCAACCACACCAGCT | |
| | GCAGATGCATGAGCAAGCTGGACGTGTACAGACAGGTGCAC | |
| | AGCATCATCAGAAGAAGCCTGCCCGCCACCCTGCCCCAGTGC | |
| | CAGGCCGCCAACAAGACCTGCCCCACCAACTACATGTGGAAC | |
| | AACCACATCTGCAGATGCCTGGCCCAGGAGGACTTCATGTTC | |
| | AGCAGCGACGCCGGCGACGACAGCACCGACGGCTTCCACGA | |
| | CATCTGCGGCCCCAACAAGGAGCTGGACGAGGAGACCTGCC | |
| | AGTGCGTGTGCAGAGCCGGCCTGAGACCCGCCAGCTGCGGCC | |
| | CCCACAAGGAGCTGGACAGAAACAGCTGCCAGTGCGTGTGC | |
| | AAGAACAAGCTGTTCCCCAGCCAGTGCGGCGCCAACAGAGA | |
| | GTTCGACGAGAACACCTGCCAGTGCGTGTGCAAGAGAACCTG | |
| | CCCCAGAAACCAGCCCCTGAACCCCGGCAAGTGCGCCTGCGA | |
| | GTGCACCGAGAGCCCCCAGAAGTGCCTGCTGAAGGGCAAGA | |
| | AGTTCCACCACCAGACCTGCAGCTGCTACAGAAGACCCTGCA | |
| | CCAACAGACAGAAGGCCTGCGAGCCCGGCTTCAGCTACAGCG | |
| | AGGAGGTGTGCAGATGCGTGCCCAGCTACTGGAAGAGACCCC | |
| | AGATGAGCTGA | |
| human VEGF- | ATGCACCTGCTGGGCTTCTTCAGCGTGGCCTGCAGCCTGCTGG | 36 |
| C full length | CCGCCGCCCTGCTGCCCGGCCCCAGAGAGGCCCCCGCCGCCG | |
| Cys156Ser | CCGCCGCCTTCGAGAGCGGCCTGGACCTGAGCGACGCCGAGC | |
| mutation | CCGACGCCGGCGAGGCCACCGCCTACGCCAGCAAGGACCTG | |
| codon | GAGGAGCAGCTGAGAAGCGTGAGCAGCGTGGACGAGCTGAT | |
| optimized 2 | GACCGTGCTGTACCCCGAGTACTGGAAGATGTACAAGTGCCA | |
| (AGS) | GCTGAGAAAGGGCGGCTGGCAGCACAACAGAGAGCAGGCCA | |
| | ACCTGAACAGCAGAACCGAGGAGACCATCAAGTTCGCCGCC | |
| | GCCCACTACAACACCGAGATCCTGAAGAGCATCGACAACGA | |
| | GTGGAGAAAGACCCAGTGCATGCCCAGAGAGGTGTGCATCG | |
| | ACGTGGGCAAGGAGTTCGGCGTGGCCACCAACACCTTCTTCA | |
| | AGCCCCCCTCCGTGAGCGTGTACAGATGCGGCGGCTGCTGCA | |
| | ACAGCGAGGGCCTGCAGTGCATGAACACCAGCACCAGCTACC | |
| | TGAGCAAGACCCTGTTCGAGATCACCGTGCCCCTGAGCCAGG | |
| | GCCCCAAGCCCGTGACCATCAGCTTCGCCAACCACACCAGCT | |
| | GCAGATGCATGAGCAAGCTGGACGTGTACAGACAGGTGCAC | |
| | AGCATCATCAGAAGAAGCCTGCCCGCCACCCTGCCCCAGTGC | |
| | CAGGCCGCCAACAAGACCTGCCCCACCAACTACATGTGGAAC | |
| | AACCACATCTGCAGATGCCTGGCCCAGGAGGACTTCATGTTC | |
| | AGCAGCGACGCCGGCGACGACAGCACCGACGGCTTCCACGA | |
| | CATCTGCGGCCCCAACAAGGAGCTGGACGAGGAGACCTGCC | |
| | AGTGCGTGTGCAGAGCCGGCCTGAGACCCGCCAGCTGCGGCC | |
| | CCCACAAGGAGCTGGACAGAAACAGCTGCCAGTGCGTGTGC | |
| | AAGAACAAGCTGTTCCCCAGCCAGTGCGGCGCCAACAGAGA | |
| | GTTCGACGAGAACACCTGCCAGTGCGTGTGCAAGAGAACCTG | |
| | CCCCAGAAACCAGCCCCTGAACCCCGGCAAGTGCGCCTGCGA | |
| | GTGCACCGAGAGCCCCCAGAAGTGCCTGCTGAAGGGCAAGA | |
| | AGTTCCACCACCAGACCTGCAGCTGCTACAGAAGACCCTGCA | |
| | CCAACAGACAGAAGGCCTGCGAGCCCGGCTTCAGCTACAGCG | |
| | AGGAGGTGTGCAGATGCGTGCCCAGCTACTGGAAGAGACCCC | |
| | AGATGAGCTGA | |
| human VEGF- | ATGCACCTGCTGGGCTTCTTCAGCGTGGCCTGCAGCCTGCTGG | 37 |
| C full length | CCGCCGCCCTGCTGCCCGGCCCCAGAGAGGCCCCCGCCGCCG | |
| Cys137A1a | CCGCCGCCTTCGAGAGCGGCCTGGACCTGAGCGACGCCGAGC | |
| mutation | CCGACGCCGGCGAGGCCACCGCCTACGCCAGCAAGGACCTG | |
| codon | GAGGAGCAGCTGAGAAGCGTGAGCAGCGTGGACGAGCTGAT | |
| optimized 2 | GACCGTGCTGTACCCCGAGTACTGGAAGATGTACAAGTGCCA | |
| (AGS) | GCTGAGAAAGGGCGGCTGGCAGCACAACAGAGAGCAGGCCA | |
| | ACCTGAACAGCAGAACCGAGGAGACCATCAAGTTCGCCGCC | |
| | GCCCACTACAACACCGAGATCCTGAAGAGCATCGACAACGA | |
| | GTGGAGAAAGACCCAGTGCATGCCCAGAGAGGTGGCCATCG | |
| | ACGTGGGCAAGGAGTTCGGCGTGGCCACCAACACCTTCTTCA | |
| | AGCCCCCCTGCGTGAGCGTGTACAGATGCGGCGGCTGCTGCA | |
| | ACAGCGAGGGCCTGCAGTGCATGAACACCAGCACCAGCTACC | |
| | TGAGCAAGACCCTGTTCGAGATCACCGTGCCCCTGAGCCAGG | |
| | GCCCCAAGCCCGTGACCATCAGCTTCGCCAACCACACCAGCT | |
| | GCAGATGCATGAGCAAGCTGGACGTGTACAGACAGGTGCAC | |
| | AGCATCATCAGAAGAAGCCTGCCCGCCACCCTGCCCCAGTGC | |
| | CAGGCCGCCAACAAGACCTGCCCCACCAACTACATGTGGAAC | |
| | AACCACATCTGCAGATGCCTGGCCCAGGAGGACTTCATGTTC | |
| | AGCAGCGACGCCGGCGACGACAGCACCGACGGCTTCCACGA | |
| | CATCTGCGGCCCCAACAAGGAGCTGGACGAGGAGACCTGCC | |
| | AGTGCGTGTGCAGAGCCGGCCTGAGACCCGCCAGCTGCGGCC | |
| | CCCACAAGGAGCTGGACAGAAACAGCTGCCAGTGCGTGTGC | |
| | AAGAACAAGCTGTTCCCCAGCCAGTGCGGCGCCAACAGAGA | |
| | GTTCGACGAGAACACCTGCCAGTGCGTGTGCAAGAGAACCTG | |
| | CCCCAGAAACCAGCCCCTGAACCCCGGCAAGTGCGCCTGCGA | |
| | GTGCACCGAGAGCCCCCAGAAGTGCCTGCTGAAGGGCAAGA | |
| | AGTTCCACCACCAGACCTGCAGCTGCTACAGAAGACCCTGCA | |
| | CCAACAGACAGAAGGCCTGCGAGCCCGGCTTCAGCTACAGCG | |
| | AGGAGGTGTGCAGATGCGTGCCCAGCTACTGGAAGAGACCCC | |
| | AGATGAGCTGA | |
| human VEGF- | GCCCACTACAACACCGAGATCCTGAAGAGCATCGACAACGA | 38 |
| C mature | GTGGAGAAAGACCCAGTGCATGCCCAGAGAGGTGTGCATCG | |
| (dNdC) wt | ACGTGGGCAAGGAGTTCGGCGTGGCCACCAACACCTTCTTCA | |
| codon | AGCCCCCCTGCGTGAGCGTGTACAGATGCGGCGGCTGCTGCA | |
| optimized 2 | ACAGCGAGGGCCTGCAGTGCATGAACACCAGCACCAGCTACC | |
| (AGS) | TGAGCAAGACCCTGTTCGAGATCACCGTGCCCCTGAGCCAGG | |
| | GCCCCAAGCCCGTGACCATCAGCTTCGCCAACCACACCAGCT | |
| | GCAGATGCATGAGCAAGCTGGACGTGTACAGACAGGTGCAC | |
| | AGCATCATCAGAAGA | |
| human VEGF- | GCCCACTACAACACCGAGATCCTGAAGAGCATCGACAACGA | 39 |
| C mature | GTGGAGAAAGACCCAGTGCATGCCCAGAGAGGTGTGCATCG | |
| (dNdC) | ACGTGGGCAAGGAGTTCGGCGTGGCCACCAACACCTTCTTCA | |
| Cys156Ser | AGCCCCCCTCCGTGAGCGTGTACAGATGCGGCGGCTGCTGCA | |
| mutation | ACAGCGAGGGCCTGCAGTGCATGAACACCAGCACCAGCTACC | |
| codon | TGAGCAAGACCCTGTTCGAGATCACCGTGCCCCTGAGCCAGG | |
| optimized 2 | GCCCCAAGCCCGTGACCATCAGCTTCGCCAACCACACCAGCT | |
| (AGS) | GCAGATGCATGAGCAAGCTGGACGTGTACAGACAGGTGCAC | |
| | AGCATCATCAGAAGA | |
| human VEGF- | GCCCACTACAACACCGAGATCCTGAAGAGCATCGACAACGA | 40 |
| C mature | GTGGAGAAAGACCCAGTGCATGCCCAGAGAGGTGGCCATCG | |
| (dNdC) | ACGTGGGCAAGGAGTTCGGCGTGGCCACCAACACCTTCTTCA | |
| Cys137A1a | AGCCCCCCTGCGTGAGCGTGTACAGATGCGGCGGCTGCTGCA | |
| mutation | ACAGCGAGGGCCTGCAGTGCATGAACACCAGCACCAGCTACC | |
| codon | TGAGCAAGACCCTGTTCGAGATCACCGTGCCCCTGAGCCAGG | |
| optimized 2 | GCCCCAAGCCCGTGACCATCAGCTTCGCCAACCACACCAGCT | |
| (AGS) | GCAGATGCATGAGCAAGCTGGACGTGTACAGACAGGTGCAC | |
| | AGCATCATCAGAAGA | |
| human VEGF- | MHLLGFFSVACSLLAAALLPGPREAPAAAAAFESGLDLSDAEP | 41 |
| C full length | DAGEATAYASKDLEEQLRSVSSVDELMTVLYPEYWKMYKCQLR | |
| wt | KGGWQHNREQANLNSRTEETIKFAAAHYNTEILKSIDNEWRKT | |
| | QCMPREVCIDVGKEFGVATNTFFKPPCVSVYRCGGCCNSEGLQC | |
| | MNTSTSYLSKTLFEITVPLSQGPKPVTISFANHTSCRCMSKLDVY | |
| | RQVHSIIRRSLPATLPQCQAANKTCPTNYMWNNHICRCLAQEDF | |
| | MFSSDAGDDSTDGFHDICGPNKELDEETCQCVCRAGLRPASCGP | |
| | HKELDRNSCQCVCKNKLFPSQCGANREFDENTCQCVCKRTCPR | |
| | NQPLNPGKCACECTESPQKCLLKGKKFHHQTCSCYRRPCTNRQ | |
| | KACEPGFSYSEEVCRCVPSYWKRPQMS | |
| human VEGF- | MHLLGFFSVACSLLAAALLPGPREAPAAAAAFESGLDLSDAEPD | 42 |
| C full length | AGEATAYASKDLEEQLRSVSSVDELMTVLYPEYWKMYKCQLR | |
| Cys156Ser | KGGWQHNREQANLNSRTEETIKFAAAHYNTEILKSIDNEWRKT | |
| mutation | QCMPREVCIDVGKEFGVATNTFFKPPSVSVYRCGGCCNSEGLQC | |
| | MNTSTSYLSKTLFEITVPLSQGPKPVTISFANHTSCRCMSKLDVY | |
| | RQVHSIIRRSLPATLPQCQAANKTCPTNYMWNNHICRCLAQEDF | |
| | MFSSDAGDDSTDGFHDICGPNKELDEETCQCVCRAGLRPASCGP | |
| | HKELDRNSCQCVCKNKLFPSQCGANREFDENTCQCVCKRTCPR | |
| | NQPLNPGKCACECTESPQKCLLKGKKFHHQTCSCYRRPCTNRQ | |
| | KACEPGFSYSEEVCRCVPSYWKRPQMS | |
| human VEGF- | MHLLGFFSVACSLLAAALLPGPREAPAAAAAFESGLDLSDAEPD | 43 |
| C full length | AGEATAYASKDLEEQLRSVSSVDELMTVLYPEYWKMYKCQLR | |
| Cys137Ala | KGGWQHNREQANLNSRTEETIKFAAAHYNTEILKSIDNEWRKT | |
| mutation | QCMPREVAIDVGKEFGVATNTFFKPPCVSVYRCGGCCNSEGLQ | |
| | CMNTSTSYLSKTLFEITVPLSQGPKPVTISFANHTSCRCMSKLDV | |
| | YRQVHSIIRRSLPATLPQCQAANKTCPTNYMWNNHICRCLAQED | |
| | FMFSSDAGDDSTDGFHDICGPNKELDEETCQCVCRAGLRPASCG | |
| | PHKELDRNSCQCVCKNKLFPSQCGANREFDENTCQCVCKRTCP | |
| | RNQPLNPGKCACECTESPQKCLLKGKKFHHQTCSCYRRPCTNR | |
| | QKACEPGFSYSEEVCRCVPSYWKRPQMS | |
| human VEGF- | AHYNTEILKSIDNEWRKTQCMPREVCIDVGKEFGVATNTFFKPP | 44 |
| C mature | CVSVYRCGGCCNSEGLQCMNTSTSYLSKTLFEITVPLSQGPKPVT | |
| (dNdC) wt | ISFANHTSCRCMSKLDVYRQVHSIIRR | |
| human VEGF- | AHYNTEILKSIDNEWRKTQCMPREVCIDVGKEFGVATNTFFKPP | 45 |
| C mature | SVSVYRCGGCCNSEGLQCMNTSTSYLSKTLFEITVPLSQGPKPVT | |
| (dNdC) | ISFANHTSCRCMSKLDVYRQVHSIIRR | |
| Cys156Ser | | |
| mutation | | |
| human VEGF- | AHYNTEILKSIDNEWRKTQCMPREVAIDVGKEFGVATNTFFKPP | 46 |
| C mature | CVSVYRCGGCCNSEGLQCMNTSTSYLSKTLFEITVPLSQGPKPVT | |
| (dNdC) | ISFANHTSCRCMSKLDVYRQVHSIIRR | |
| Cys137Ala | | |
| mutation | | |
| 5′-non-coding- | CCGCGGTTAGAAAAAATACGGGTAGAACCGCCACC | 47 |
| region-for- | | |
| NDV | | |
| 5′-non-coding- | GTCGACATTTTTAATTAAAATAGGGTGGGGAAGGTACCGCCA | 48 |
| region-for- | CC | |
| APMV4 | | |
| Nucleotide | cccgctccttccaccatgcacttgctgggcttctggtctctggcg | 49 |
| sequence for | tgttccctgctcgccgctgcgctgctgccggggccccgcgacgcg | |
| Canis lupus | cccgccgccgccgccgccttcgaatcgggactcggcttctccgac | |
| familiaris | gcggagcccgacgcgggcgaggcccaggcgtatgcaggcaaagat | |
| VEGF-C | ttggaggaacagttgcgatcagcgtccagtgtagatgaactcatg | |
| GenBank No. | accgtactctacccagaatattggaaaatgtacaagtgtcagtta | |
| XM_540047.6 | aggaaaggcggctggcagcgtaataaagaacagcccaacatcagt | |
| | gcaagaacagaagagactataaaatttgctgcagcacattataat | |
| | gcagagatcttgaaaagtattgataatgagtggagaaaaactcag | |
| | tgcataccacgtgaggtgtgtatagatgtggggaaggagtttgga | |
| | gcagcaacaaacaccttctttaaacctccatgtgtgtccgtctac | |
| | agatgtggtggctgctgtaacagcgagggcctacagtgtatgaac | |
| | accagcacaagccacctcagcaagacgttgtttgaaattacagtg | |
| | cctctctctcaaggccccaaaccagtaacaatcagttttgccaat | |
| | cacacttcctgccgatgcatgtctaaactggacgtttacagacaa | |
| | gttcattccattattagacgttccctgccagcaacactaccacag | |
| | tgccaagctgcaaacaagacttgccccacaaattacatctggaat | |
| | aatcatctctgcagatgcctggctcagcaagattttatttttgcc | |
| | tcaaattctggagatgactctacagatggattccatgacatctgt | |
| | ggacctaacaaggagctagatgaagaaacgtgtcagtgtgtctgc | |
| | agaggggggctccggccttccagctgtggaccccacaaggaacta | |
| | gacagaaactcctgccagtgtgtctgtaaaaacaaactgttaccc | |
| | aactcgtgtggggccaacagagaatttgatgaaaacacgtgccag | |
| | tgcgtatgtaaaagaacctgcccaagaaatcaacccctaaaccct | |
| | ggaaaatgtgcctgtgagtgtacagaaaattcacagaaatgcttc | |
| | ttaaaaggaaagaaatttcaacatcaaacatgcagctgttacaga | |
| | agaccgtgtacaaaccgactgaggcattgtgagcaaggacttata | |
| | tttagtgaagaagtatgtcgctgtgtcccttcatactggaaaaga | |
| | ccacagatgaactaagactgta | |
| | ctgttttccagtttgccatttctttatcttggaaaaccgtgttgc | |
| | cacattagaactatctgtgaacacagagaccttggtgggaccatg | |
| | gagacagagacagaagtcagtgtttgctgacctgtgtggataact | |
| | ttacagaaacggactggagctcatctgcaaaagacctcttttaat | |
| | gactggtttttctgccaatgaccagacagctgaggtttttctctt | |
| | gtgattaaaaaaaaaaaataatgactatataatttatttccacta | |
| | aaaatattgtttctgcattcatgtttatagcaataacaattggta | |
| | aagctcactgtgatcaatatttttatatcatgcaaaatatgttta | |
| | aaataaaatgaaaattgtattataaa | |
| Nucleotide | cccgctccttccaccatgcacttgctgggcttctggtctctggcg | 50 |
| sequence for | tgttccctgctcgccgctgcgctgctgccggggccccgcgacgcg | |
| Canis lupus | cccgccgccgccgccgccttcgaatcgggactcggcttctccgac | |
| dingo VEGF- | gcggagcccgacgcgggcgaggcccaggcgtatgcaggcaaagat | |
| C (GenBank | ttggaggaacagttgcgatcagcgtccagtgtagatgaactcatg | |
| Accession | accgtactctacccagaatattggaaaatgtacaagtgtcagtta | |
| NO. | aggaaaggcggctggcagcgtaataaagaacagcccaacatcagt | |
| XM_0254340 | gcaagaacagaagagactataaaatttgctgcagcacattataat | |
| 44) | gcagagatcttgaaaagtattgataatgagtggagaaaaactcag | |
| | tgcataccacgtgaggtgtgtatagatgtggggaaggagtttgga | |
| | gcagcaacaaacaccttctttaaacctccatgtgtgtccgtctac | |
| | agatgtggtggctgctgtaacagcgagggcctacagtgtatgaac | |
| | accagcacaagccacctcagcaagacgttgtttgaaattacagtg | |
| | cctctctctcaaggccccaaaccagtaacaatcagttttgccaat | |
| | cacacttcctgccgatgcatgtctaaactggatgtttacagacaa | |
| | gttcattccattattagacgttccctgccagcaacactaccacag | |
| | tgccaagctgcaaacaagacttgccccacaaattacatctggaat | |
| | aatcatctctgcagatgcctggctcagcaagattttatttttgcc | |
| | tcaaattctggagatgactctacagatggattccatgacatctgt | |
| | ggacctaacaaggagctagatgaagaaacgtgtcagtgtgtctgc | |
| | agaggggggctccggccttccagctgtggaccccacaaggaacta | |
| | gacagaaactcctgccagtgtgtctgtaaaaacaa | |
| | actgttacccaactcgtgtggggccaacagagaatttgatgaaaa | |
| | cacgtgccagtgcgtatgtaaaagaacctgcccaagaaatcaacc | |
| | cctaaaccctggaaaatgtgcctgtgagtgtacagaaaattcaca | |
| | gaaatgcttcttaaaaggaaagaaatttcaacatcaaacatgcag | |
| | ctgttacagaagaccgtgtacaaaccgactgaggcattgtgagca | |
| | aggacttatatttagtgaagaagtatgtcgctgtgtcccttcata | |
| | ctggaaaagaccacagatgaactaagactgtactgttttccagtt | |
| | tgccatttctttatcttggaaaaccgtgttgccacattagaacta | |
| | tctgtgaacacagagaccttggtgggaccatggagacagagacag | |
| | aagtcagtgtttgctgacctgtgtggataactttacagaaacgga | |
| | ctggagctcatctgcaaaagacctcttttaatgactggtttttct | |
| | gccaatgaccagacagctgaggtttttctcttgtgattaaaaaaa | |
| | aaaaataatgactatataatttatttccactaaaaatattgtttc | |
| | tgcattcatgtttatagcaataacaattggtaaagctcactgtga | |
| | tcaatatttttatatcatgcaaaatatgtttaaaataaaatgaaa | |
| | attgtattataaa | |
| Canis Lupus | MHLLGFWSLACSLLAAALLPGPRDAPAAAAAFESGLGFSDAEP | 51 |
| Familiaris | DAGEAQAYAGKDLEEQLRSASSVDELMTVLYPEYWKMYKCQL | |
| VEGF-C | RKGGWQRNKEQPNISARTEETIKFAAAHYNAEILKSIDNEWRKT | |
| (GenBank | QCIPREVCIDVGKEFGAATNTFFKPPCVSVYRCGGCCNSEGLQC | |
| Accession No. | MNTSTSHLSKTLFEITVPLSQGPKPVTISFANHTSCRCMSKLDVY | |
| XP_540047.2) | RQVHSIIRRSLPATLPQCQAANKTCPTNYIWNNHLCRCLAQQDFI | |
| | FASNSGDDSTDGFHDICGPNKELDEETCQCVCRGGLRPSSCGPH | |
| | KELDRNSCQCVCKNKLLPNSCGANREFDENTCQCVCKRTCPRN | |
| | QPLNPGKCACECTENSQKCFLKGKKFQHQTCSCYRRPCTNRLR | |
| | HCEQGLIFSEEVCRCVPSYWKRPQMN | |
| Canis Lupus | MHLLGFWSLACSLLAAALLPGPRDAPAAAAAFESGLGFSDAEP | 52 |
| Dingo: | DAGEAQAYAGKDLEEQLRSASSVDELMTVLYPEYWKMYKCQL | |
| VEGF-C | RKGGWQRNKEQPNISARTEETIKFAAAHYNAEILKSIDNEWRKT | |
| (GenBank | QCIPREVCIDVGKEFGAATNTFFKPPCVSVYRCGGCCNSEGLQC | |
| Accession No. | MNTSTSHLSKTLFEITVPLSQGPKPVTISFANHTSCRCMSKLDVY | |
| XP 02528982 | RQVHSIIRRSLPATLPQCQAANKTCPTNYIWNNHLCRCLAQQDFI | |
| 9.1) | FASNSGDDSTDGFHDICGPNKELDEETCQCVCRGGLRPSSCGPH | |
| | KELDRNSCQCVCKNKLLPNSCGANREFDENTCQCVCKRTCPRN | |
| | QPLNPGKCACECTENSQKCFLKGKKFQHQTCSCYRRPCTNRLR | |
| | HCEQGLIFSEEVCRCVPSYWKRPQMN | |
| Human | aagacacatg cttctgcaag cttccatgaa ggttgtgcaa aaaagtttca | 96 |
| vascular | atccagagtt gggttccagc tttctgtagc tgtaagcatt ggtggccaca | |
| endothelial | ccacctcctt acaaagcaac tagaacctgc ggcatacatt ggagagattt | |
| growth factor- | ttttaatttt ctggacatga agtaaattta gagtgctttc taatttcagg | |
| D (VEGF-D) | tagaagacat gtccaccttc tgattatttt tggagaacat tttgattttt | |
| GenBank No. | ttcatctctc tctccccacc cctaagattg tgcaaaaaaa gcgtaccttg | |
| NM_004469.5 | cctaattgaa ataatttcat tggattttga tcagaactga ttatttggtt | |
| Uni-Prot. | ttctgtgtga agttttgagg tttcaaactt tccttctgga gaatgccttt | |
| 043915 | tgaaacaatt ttctctagct gcctgatgtc aactgcttag taatcagtgg | |
| | atattgaaat attcaaaatg tacagagagt gggtagtggt gaatgttttc | |
| | atgatgttgt acgtccagct ggtgcagggc tccagtaatg aacatggacc | |
| | agtgaagcga tcatctcagt ccacattgga acgatctgaa cagcagatca | |
| | gggctgcttc tagtttggag gaactacttc gaattactca ctctgaggac | |
| | tggaagctgt ggagatgcag gctgaggctc aaaagtttta ccagtatgga | |
| | ctctcgctca gcatcccatc ggtccactag gtttgcggca actttctatg | |
| | acattgaaac actaaaagtt atagatgaag aatggcaaag aactcagtgc | |
| | agccctagag aaacgtgcgt ggaggtggcc agtgagctgg ggaagagtac | |
| | caacacattc ttcaagcccc cttgtgtgaa cgtgttccga tgtggtggct | |
| | gttgcaatga agagagcctt atctgtatga acaccagcac ctcgtacatt | |
| | tccaaacagc tetttgagat atcagtgcct ttgacatcag tacctgaatt | |
| | agtgcctgtt aaagttgcca atcatacagg ttgtaagtgc ttgccaacag | |
| | ccccccgcca tccatactca attatcagaa gatccatcca gatccctgaa | |
| | gaagatcgct gttcccattc caagaaactc tgtcctattg acatgctatg | |
| | ggatagcaac aaatgtaaat gtgttttgca ggaggaaaat ccacttgctg | |
| | gaacagaaga ccactctcat ctccaggaac cagctctctg tgggccacac | |
| | atgatgtttg acgaagatcg ttgcgagtgt gtctgtaaaa caccatgtcc | |
| | caaagatcta atccagcacc ccaaaaactg cagttgcttt gagtgcaaag | |
| | aaagtctgga gacctgctgc cagaagcaca agctatttca cccagacacc | |
| | tgcagctgtg aggacagatg cccctttcat accagaccat gtgcaagtgg | |
| | caaaacagca tgtgcaaagc attgccgctt tccaaaggag aaaagggctg | |
| | cccaggggcc ccacagccga aagaatcctt gattcagcgt tccaagttcc | |
| | ccatccctgt catttttaac agcatgctgc tttgccaagt tgctgtcact | |
| | gtttttttcc caggtgttaa aaaaaaaatc cattttacac agcaccacag | |
| | tgaatccaga ccaaccttcc attcacacca gctaaggagt ccctggttca | |
| | ttgatggatg tcttctagct gcagatgcct ctgcgcacca aggaatggag | |
| | aggaggggac ccatgtaatc cttttgttta gttttgtttt tgttttttgg | |
| | tgaatgagaa aggtgtgctg gtcatggaat ggcaggtgtc atatgactga | |
| | ttactcagag cagatgagga aaactgtagt ctctgagtcc tttgctaatc | |
| | gcaactcttg tgaattattc tgattctttt ttatgcagaa tttgattcgt | |
| | atgatcagta ctgactttct gattactgtc cagcttatag tcttccagtt | |
| | taatgaacta ccatctgatg tttcatattt aagtgtattt aaagaaaata | |
| | aacaccatta ttcaagcca | |
| Canine | ttttctgtgt gtccgtggca gtcgatgtgt gaacatctga ggtcccttcc | 97 |
| VEGF-D | tgagcattgc gatttccatg caacattcat gcctgtgtgc tggggtttca | |
| Canis lupus | cgttacaggt tatctgcatt aaaataacag cagtcctgat ggtttgagtc | |
| familiaris | agttttcaaa actgccctgc tattggtagg gacgcgacag gattacagcc | |
| GenBank No. | aagacttccc tgcattttct gccaaagtct ctgtcagatt taagacacat | |
| XM_548869.5 | gcttccgcaa ccttccatga gggttgtaaa aaaagtctga atccagaatt | |
| | gggttccagc cttctgtggc tgcaaacatt ggtggccaca ccacctcctt | |
| | acaaagcaac tagaacctgg ggcagagggt ggagagattt ttttttttaa | |
| | tttgctggac atgaaatgaa tttagagtgc tttttcgtgt caagtggaag | |
| | tcatgtccac ctcctgatta tttttggagc atgagtgcat ttaatttttt | |
| | ttcatctctc tccccgcata agattgagca aaaacgttcc ttgactaatt | |
| | gaagtcattt cattggattt tgatcacaac tgattatttg ggttttttcc | |
| | atgtgaagtc ttggggtttc gaactttcct tctggagaat gccttttgaa | |
| | acagttttct ctagctgcct gatgtcaact gcttggtaat cggtggacat | |
| | taaaatactc aaaatgtaca gacagtgggc cgtggtgaat gttttcatga | |
| | tgtcttatct acagttggtg cacagctcca gttatgagca tggaccagtg | |
| | aagcgggcat ctcggtcaac gttagagcgg tctgaacagc agattagggc | |
| | agcttctggt ttggaagaac tgctgcggat cacacacttc gaggactgga | |
| | agctctggag atgccgactg aagctcaaaa gtttgaccag cacagactct | |
| | cgctcagcat cccatcgggc caccaggttt gcggcaactt tctatgacat | |
| | tgaaacccta aaagtcatag acgaggagtg gcagcggacg cagtgcagcc | |
| | cccgggagac gtgcgtggag gtggccagcg agctggggag gagcaccgac | |
| | acgttcttca agccgccctg cgtgaacgtg ttccgctgtg gcggctgctg | |
| | caacgaggag agcctcgtct gtatgaacac gagcacctcc tacgtctcca | |
| | aacagctctt tgagatatca gtgcctttga cttcagtacc tgaattagtg | |
| | cctgttaagg tggccaacca tacaggttgt aagtgcctgc caacggctcc | |
| | ccgccatcca tactccatta tcagaagatc catccagatc ccagaagaag | |
| | atcactgttc ccattccaag caactctgtc ctgttgacat gctatgggat | |
| | agcgacaaat gtaaatgtgt tttacaggag gagaatccac tcgttggaat | |
| | ggaagaccac tctcacctcc aggaactggc tctctgcggg ccgcacatga | |
| | agtttgacga cgatcgttgc gagtgtgtct gtaaaacacc gtgtcccaga | |
| | gatctcatcc agcacccaga aaactgcagt tgcatggagt gcagagagag | |
| | cctggagagc tgctgccaga agcacaagat atttcacgca gacacctgca | |
| | gctgtgagga cagatgtccc tttcacacca gaacatgtgc gcatggaaga | |
| | ccagcatgtg caaagcactg ccgctttccg aaggagaaaa gggctgccta | |
| | tgggttccat ggtcaagaaa atccttgact caacttggtt cctgagttcc | |
| | ccatccctaa cattttaaac agcatgctgc tttgccaagt tgctgtcact | |
| | gattgttttt ttttccccac gtacaagaaa aaaaaatctg ttttacccag | |
| | tcccacaatg aattcagacc acccttccat tacacaccag ctgaggcttc | |
| | cctggttcac tgacagatga ctgccaactg aagatgcccc tgcacagcag | |
| | gatggagagg agggaacctg tagcagcccc ctcccttttt tttttggtga | |
| | atgttaaagg tctgatcatc ctagaatcac aggggccata aaattgatta | |
| | ctcaaagcca acaaggcaat tttatagtct ccaagtcctt cgctaatgca | |
| | gctgtcttgt gaattcttct gactctttat tatgcagatt ttgatttgta | |
| | tgatcagcac tgattttctg attactgtcc agcttgtagt tttgagttta | |
| | ctgaactact gtctgttgtt tcatatttaa gtgtatttaa agaaaataaa | |
| | caccattatt caagccgtgg aa | |
| Canine | ttttctgtgt gtccgtggca gtcgatgtgt gaacatctga ggtcccttcc | 98 |
| VEGF-D | tgagcattgc gatttccatg caacattcat gcctgtgtgc tggggtttca | |
| Canis lupus | cgttacaggt tatctgcatt aaaataacag cagtcctgat ggtttgagtc | |
| dingo | agttttcaaa actgccctgc tattggtagg gacgcgacag gattacagcc | |
| GenBank No. | aagacttccc tgcattttct gccaaagtct ctgtcagatt taagacacat | |
| XM_0254370 | gcttccgcaa ccttccatga gggttgtaaa aaaagtctga atccagaatt | |
| 83 | gggttccagc cttctgtggc tgcaaacatt ggtggccaca ccacctcctt | |
| | acaaagcaac tagaacctgg ggcagagggt ggagagattt ttttttttaa | |
| | tttgctggac atgaaatgaa tttagagtgc tttttcgtgt caagtggaag | |
| | tcatgtccac ctcctgatta tttttggagc atgagtgcat ttaatttttt | |
| | ttcatctctc tccccgcata agattgagca aaaacgttcc ttgactaatt | |
| | gaagtcattt cattggattt tgatcacaac tgattatttg ggttttttcc | |
| | atgtgaagtc ttggggtttc gaactttcct tctggagaat gccttttgaa | |
| | acagttttct ctagctgcct gatgtcaact gcttggtaat cggtggacat | |
| | taaaatactc aaaatgtaca gacagtgggc cgtggtgaat gttttcatga | |
| | tgtcttatct acagttggtg cacagctcca gttatgagca tggaccagtg | |
| | aagcgggcat ctcggtcaac gttagagcgg tctgaacagc agattagggc | |
| | agcttctggt ttggaagaac tgctgcggat cacacacttc gaggactgga | |
| | agctctggag atgccgactg aagctcaaaa gtttgaccag cacagactct | |
| | cgctcagcat cccatcgggc caccaggttt gcggcaactt tctatgacat | |
| | tgaaacccta aaagtcatag acgaggagtg gcagcggacg cagtgcagcc | |
| | cccgggagac gtgcgtggag gtggccagcg agctggggag gagcaccgac | |
| | acgttcttca agccgccctg cgtgaacgtg ttccgctgtg gcggctgctg | |
| | caacgaggag agcctcgtct gtatgaacac gagcacctcc tacgtctcca | |
| | aacagctctt tgagatatca gtgcctttga cttcagtacc tgaattagtg | |
| | cctgttaagg tggccaacca tacaggttgt aagtgcctgc caacggctcc | |
| | ccgccatcca tactccatta tcagaagatc catccagatc ccagaagaag | |
| | atcactgttc ccattccaag caactctgtc ctgttgacat gctatgggat | |
| | agcgacaaat gtaaatgtgt tttacaggag gagaatccac tcgttggaat | |
| | ggaagaccac tctcacctcc aggaactggc tctctgcggg ccgcacatga | |
| | agtttgacga cgatcgttgc gagtgtgtct gtaaaacacc gtgtcccaga | |
| | gatctcatcc agcacccaga aaactgcagt tgcatggagt gcagagagag | |
| | cctggagagc tgctgccaga agcacaagat atttcacgca gacacctgca | |
| | gctgtgagga cagatgtccc tttcacacca gaacatgtgc gcatggaaga | |
| | ccagcatgtg caaagcactg ccgctttccg aaggagaaaa gggctgccta | |
| | tgggttccat ggtcaagaaa atccttgact caacttggtt cctgagttcc | |
| | ccatccctaa cattttaaac agcatgctgc tttgccaagt tgctgtcact | |
| | gattgttttt ttttccccac gtacaagaaa aaaaaatctg ttttacccag | |
| | tcccacaatg aattcagacc acccttccat tacacaccag ctgaggcttc | |
| | cctggttcac tgacagatga ctgccaactg aagatgcccc tgcacagcag | |
| | gatggagagg agggaacctg tagcagcccc ctcccttttt ttttggtgaa | |
| | tgttaaaggt ctgatcatcc tagaatcaca ggggccataa aattgattac | |
| | tcaaagccaa caaggcaatt ttatagtctc caagtccttc gctaatgcag | |
| | ctgtcttgtg aattcttctg actctttatt atgcagattt tgatttgtat | |
| | gatcagcact gattttctga ttactgtcca gcttgtagtt ttgagtttac | |
| | tgaactactg tctgttgttt catatttaag tgtatttaaa gaaaataaac | |
| | accattattc aagccgtgga a | |
| Canis Lupus | MYRQWAVVNVFMMSYLQLVHSSSYEHGPVKRASRSTLERSEQQIRAASGLEELLRITH | 99 |
| Dingo: | FEDWKLWRCRLKLKSLTSTDSRSASHRATRFAATFYDIETLKVIDEEWQRTQCSPRETC | |
| VEGF-D | VEVASELGRSTDTFFKPPCVNVFRCGGCCNEESLVCMNTSTSYVSKQLFEISVPLTSVP | |
| (GenBank | ELVPVKVANHTGCKCLPTAPRHPYSIIRRSIQIPEEDHCSHSKQLCPVDMLWDSDKCKC | |
| Accession No. | VLQEENPLVGMEDHSHLQELALCGPHMKFDDDRCECVCKTPCPRDLIQHPENCSCMECR | |
| XP 02529286 | ESLESCCQKHKIFHADTCSCEDRCPFHTRTCAHGRPACAKHCRFPKEKRAAYGFHGQEN | |
| 8.1) | P | |
| Canis Lupus | MYRQWAVVNVFMMSYLQLVHSSSYEHGPVKRASRSTLERSEQQIRAASGLEELLRITHF | 100 |
| Familiaris | EDWKLWRCRLKLKSLTSTDSRSASHRATRFAATFYDIETLKVIDEEWQRTQCSPRETCV | |
| VEGF-D | EVASELGRSTDTFFKPPCVNVFRCGGCCNEESLVCMNTSTSYVSKQLFEISVPLTSVPE | |
| (GenBank | LVPVKVANHTGCKCLPTAPRHPYSIIRRSIQIPEEDHCSHSKQLCPVDMLWDSDKCKCV | |
| Accession No. | LQEENPLVGMEDHSHLQELALCGPHMKFDDDRCECVCKTPCPRDLIQHPENCSCMECRE | |
| XP_548869.2) | SLESCCQKHKIFHADTCSCEDRCPFHTRTCAHGRPACAKHCRFPKEKRAAYGFHGQEN | |
| | P | |
| Human ANAC | FAATFYDIETLKVIDEEWQRTQCSPRETCVEVASELGKSTNTFFK | 101 |
| VEGF-D | PPCVNVFRCGGCCNE | |
| (mature, | ESLICMNTSTSYISKQLFEISVPLTSVPELVPVKVANHTGCKCLPT | |
| processed | APRHPYSIIRR | |
| VEGF-D) | | |
| Human | SIQIPEEDRCSHSKKLCPIDMLWDSNKCKCVLQEENPLAGTEDHS | 102 |
| VEGF-D C- | HLQEPALCGPHMMFD | |
| terminal | EDRCECVCKTPCPKDLIQHPKNCSCFECKESLETCCQKHKLFHP | |
| propeptide | DTCSCEDRCPFHTRPC | |
| | ASGKTACAKHCRFPKEKRAAQGPHSRKNP | |
| Human | SSNEHGPVKRSSQSTLERSEQQIRAASSLEELLRITHSEDWKLWR | 103 |
| VEGF-D N- | CRLRLKSFTSMDSRS | |
| terminal | ASHRSTR | |
| propeptide | | |
| Human | MYREWVVVNV FMMLYVQLVQ GSSNEHGPVK RSSQSTLERS EQQIRAASSL | 104 |
| VEGF-D | EELLRITHSE DWKLWRCRLR LKSFTSMDSR SASHRSTRFA ATFYDIETLK | |
| (UniProt: | VIDEEWQRTQ CSPRETCVEV ASELGKSTNT FFKPPCVNVF RCGGCCNEES | |
| 043915) | LICMNTSTSY ISKQLFEISV PLTSVPELVP VKVANHTGCK CLPTAPRHPY | |
| | SIIRRSIQIP EEDRCSHSKK LCPIDMLWDS NKCKCVLQEE NPLAGTEDHS | |
| | HLQEPALCGP HMMFDEDRCE CVCKTPCPKD LIQHPKNCSC FECKESLETC | |
| | CQKHKLFHPD TCSCEDRCPF HTRPCASGKT ACAKHCRFPK EKRAAQGPHS | |
| | RKNP | |
| Human | MYREWVVVNVFMMLYVQLVQG | 105 |
| VEGF-D | | |
| signal peptide | | |

TABLE 4
Other Sequences

| | | SEQ |
|---|---|---|
| | | ID |
| Description | Sequence | NO: |
| Cleavage Site | S116: ¹¹¹H-N-R-T-K-S/F¹¹⁷ | 91 |
| Modification | | |
| Cleavage Site | S116K: ¹¹¹H-N-K-T-K-S/F¹¹⁷ | 92 |
| Modification | | |
| Cleavage Site | S116M: ¹¹¹H-N-R-M-K-S/F¹¹⁷ | 93 |
| Modification | | |
| Cleavage Site | S116KM: ¹¹¹H-N-K-M-K-S/F-I¹¹⁸ | 94 |
| Modification | | |
| Cleavage Site | R116: ¹¹¹H-N-R-T-K-R/F-I¹¹⁸ | 95 |
| Modification | | |

### 6. EXAMPLES

### 6.1 Example 1: Construction of Recombinant Paramyxovirus-VEGF-C

Rescue of Recombinant Newcastle Disease Virus (rNDV).

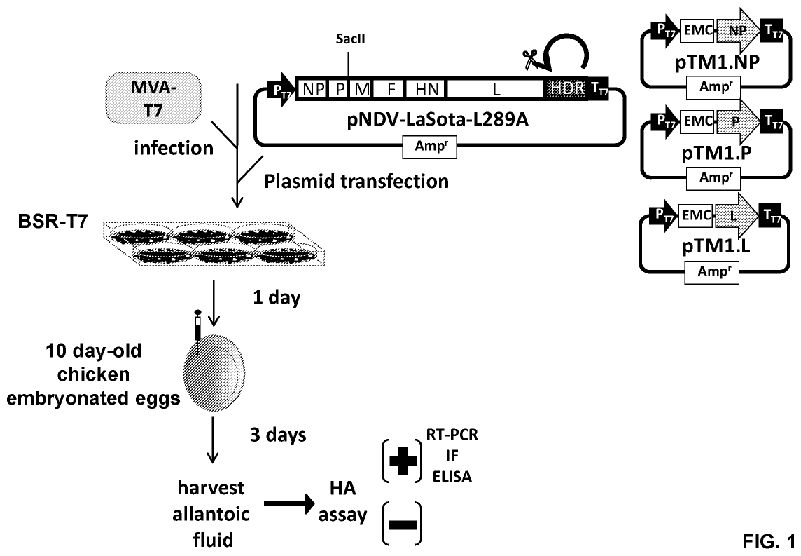

The virus was rescued following a very well stablished protocol, already described (Ayllon J, Garcia-Sastre A, Martinez-Sobrido L. 2013, Rescue of recombinant Newcastle disease virus from cDNA. J Vis Exp. 2013 Oct. 11; (80). doi: 10.3791/50830), with a few modifications. A schematic of the protocol is shown in FIG. 1. Briefly, BSR-T7 cells in a 6 well plate were infected with a recombinant vaccinia virus that expresses the T7 RNA polymerase (MVA-T7) and transfected with plasmids pNDV-LaSota-L289A (SEQ ID NO: 85), pTM1.NP, pTM1.P and pTM1.L. The following day supernatant and cells were harvested from the plate and inoculated into 10 days-old embryonated chicken eggs to amplify the rescued virus. After 3 days of incubation, the allantoic fluid was harvested and analyzed by hemagglutination assay to detect the presence of rNDV. Hemagglutinin (HA) positive samples were further characterized to confirm the presence and expression of the inserted foreign genes. See Ayllon et al., Rescue of Recombinant Newcastle Disease Virus from cDNA. J. Vis. Exp. (80), e50830, doi:10.3791/50830 (2013) for a description of the methodology used to rescue recombinant NDV.

Construction of a Rescue Plasmid to Obtain a rNDV Expressing the Mouse Vascular Endothelial Growth Factor-C (mVEGF-C).

The Open Reading Frame of mouse VEGF-C was amplified from a synthetic cDNA corresponding to a codon optimized VEGF-C (SEQ ID NO: 7) by PCR using primers that incorporate the following sequences: Forward primer: Sac II restriction site+NDV regulatory sequences (gene end+intergene+gene start)+Kozac sequences for optimal translation. Reverse primer: additional nucleotides (rule of 6)+Sac II restriction site. See, e.g., Table 1 (SEQ ID NOS: 65 and 66) for primer sequences.

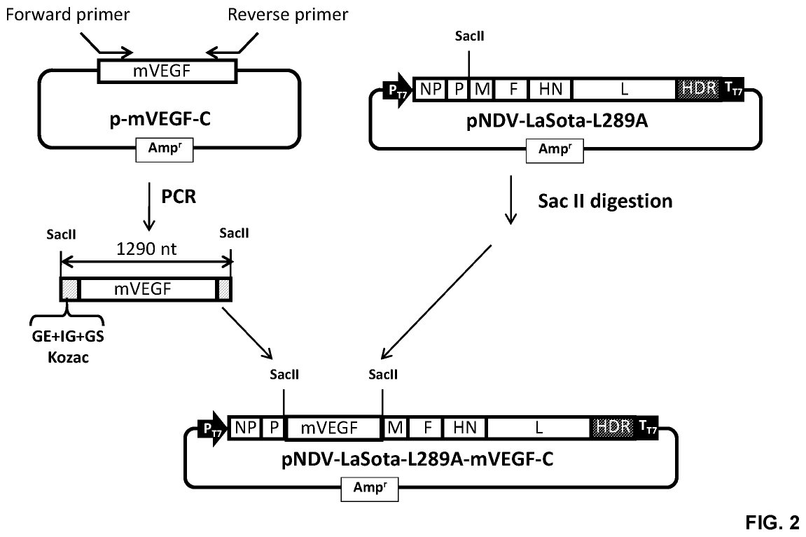

The size of the amplified insert was compliant with the rule of 6 to warrant efficient encapsidation of the genome by the viral nucleoprotein (NP). The PCR product was cloned into the unique Sac II site of the pNDV-LaSota-L289A plasmid (see, e.g., SEQ ID NO: 85). A schematic showing the construction of the rescue plasmid is shown in FIG. 2. After confirmation that the insert was present, the plasmid was used to rescue a rNDV expressing mVEGF-C as described above. Presence of the additional gene in the viral genome was confirmed by RT-PCR and expression of mVEGF-C was confirmed by immunological assays (ELISA and immunofluorescence) on infected Vero cells.

Cloning of a Full Length cDNA of the Genome of Avian Paramyxovirus 4 (APMV4).

Viral RNA was purified from a preparation of APMV4 (strain Duck/Hong Kong/D3/1975) amplified in embryonated eggs and concentrated by ultracentrifugation through a sucrose cushion. The genome sequence was confirmed by deep sequencing. Rapid Amplification of cDNA Ends (RACE) was used to confirm the sequence of the 5′ and 3′ ends. See SEQ ID NO: 86 for the full length genome of APMV4 from RNA sequencing.

The purified RNA was used as template in RT-PCR to amplify partial fragments corresponding to each viral gene. Primers were designed to introduce unique restriction sites at non-conserved parts of the intergenic regions. See Table 1 and SEQ ID NOS: 53-56 and 67-76 for primer sequences. Next, the amplified RT-PCR fragments were cloned in the multicloning site of plasmid pUC-18 to generate intermediate plasmids pUC-APMV4-1 (with genes NP, P and M), pUC-APMV4-2 (with genes F and HN) and pUC-APMV4-3 (with gene L). The inserts of plasmids 1 and 2 were ligated to generate plasmid pUC-APMV4-1+2 and finally the complete genome was assembled in plasmid pUC-APMV4-1+2+3.

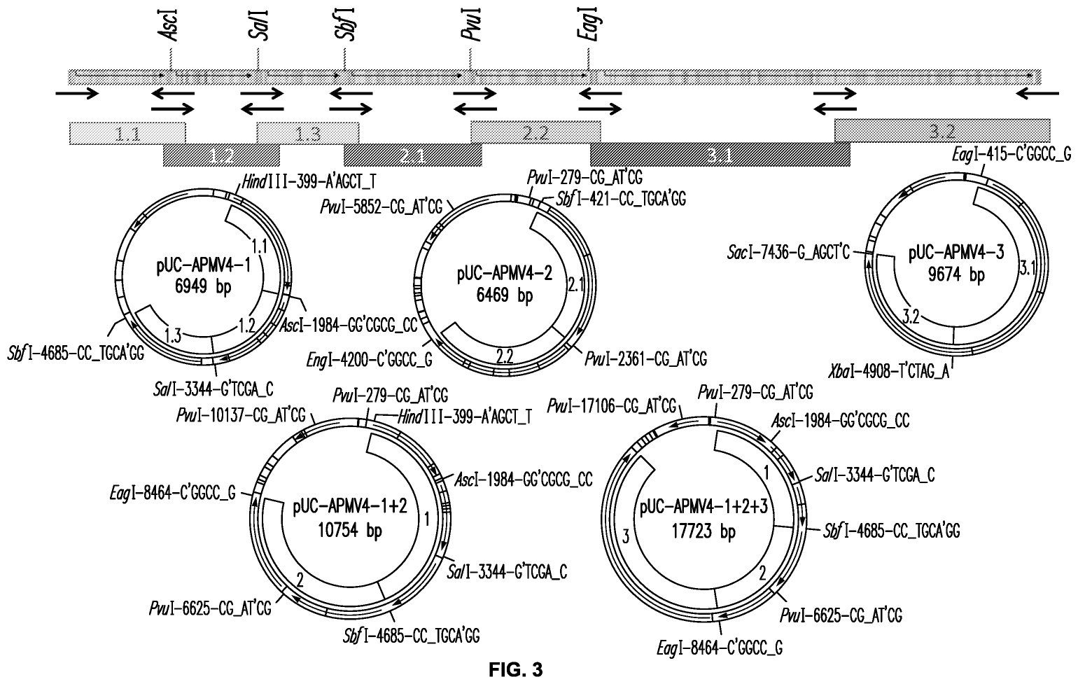

Next, the full length cDNA of the APMV4 viral genome was subcloned into the final rescue plasmid pRz-APMV4, under the control of the T7 RNA polymerase promoter and terminator sequences and flanked by ribozymes to generate the correct 5′ and 3′ ends. In this plasmid the Sal I site engineered between the viral genes P and M is not unique. A schematic showing the cloning of a full-length cDNA of the APMV4 genome is shown in FIG. 3.

Cloning of Helper Plasmids Expressing the APMV4 Proteins NP, P and L Under the Control of the T7 RNA Polymerase.

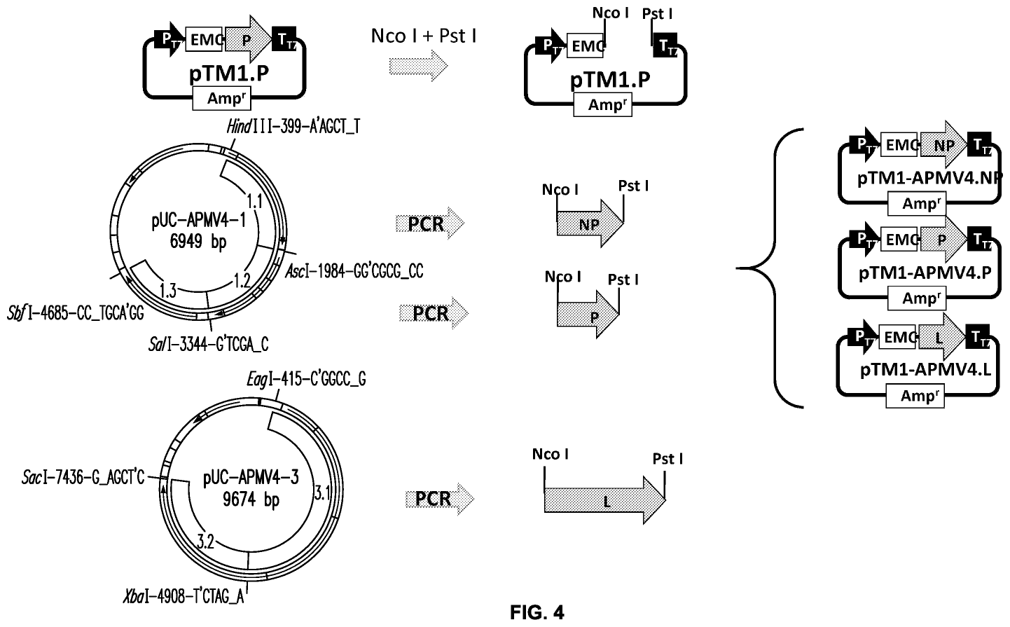

To generate the APMV4 helper plasmids, the open reading frames of viral genes NP, P and L were amplified using as templates plasmids pUC-APMV4-1 (for genes NP and P) and pUC-APMV4-3 (for gene L). See, e.g., Table 1 and SEQ ID NOS: 57-62 for primer sequences. The PCR amplified products were cloned into the expression plasmid pTM1 using the restriction sites Nco I and Pst I. A schematic showing the protocol for the cloning of the helper plasmids is shown in FIG. 4.

Rescue of Recombinant APMV4.

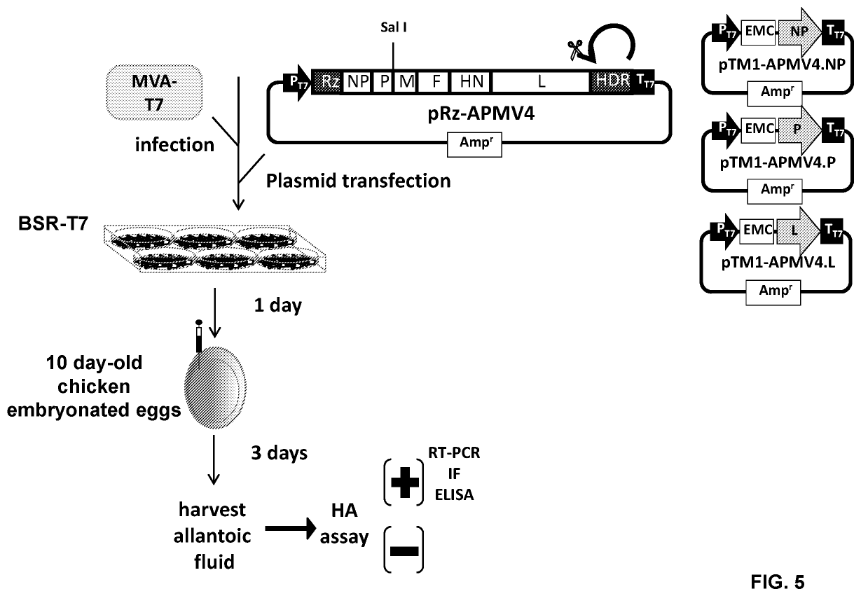

The virus is rescued following the same protocol described above for rNDV (schematic of the protocol shown in FIG. 5). Briefly, BSR-T7 cells in a 6 well plate are infected with a recombinant vaccinia virus that expresses the T7 RNA polymerase (MVA-T7) and transfected with plasmids pRz-APMV4, pTM1-APMV4.NP, pTM1-APMV4.P and pTM1-APMV4.L. The following day supernatant and cells are harvested from the plate and inoculated into 10 days-old embryonated chicken eggs to amplify the rescued virus. After a three-day incubation, the allantoic fluid is harvested and analyzed by hemagglutination assay to detect the presence of rAPMV4. HA positive samples are further characterized to confirm the presence and expression of the inserted foreign genes.

Construction of a Rescue Plasmid to Obtain a rAPMV4 Expressing the Mouse Vascular Endothelial Growth Factor-C (mVEGF-C).

The rescue plasmid to obtain a rAPMV4-mVEGF-C was prepared as described for the rNDV above, but using as template a synthetic sequence with a codon-optimized mVEGF-C gene. The codon-optimized sequence was designed using the web based tool at www.encorbio.com/protocols/Codon.htm.

Next, the optimized Open Reading Frame of mVEGF-C was amplified by PCR using primers that incorporate the following sequences: Forward primer: Sal I restriction site+APMV regulatory sequences (gene end+intergene+gene start)+Kozac sequences for optimal translation. Reverse primer: additional nucleotides (rule of 6)+Sal I restriction site. See, e.g., Table 1 and SEQ ID Nos: 63 and 64 for primer sequences, SEQ ID NO: 13 for codon optimized mVEGF-C sequence, and SEQ ID NO: 89 for codon optimized mouse VEGF-C sequence plus regulatory sequences.

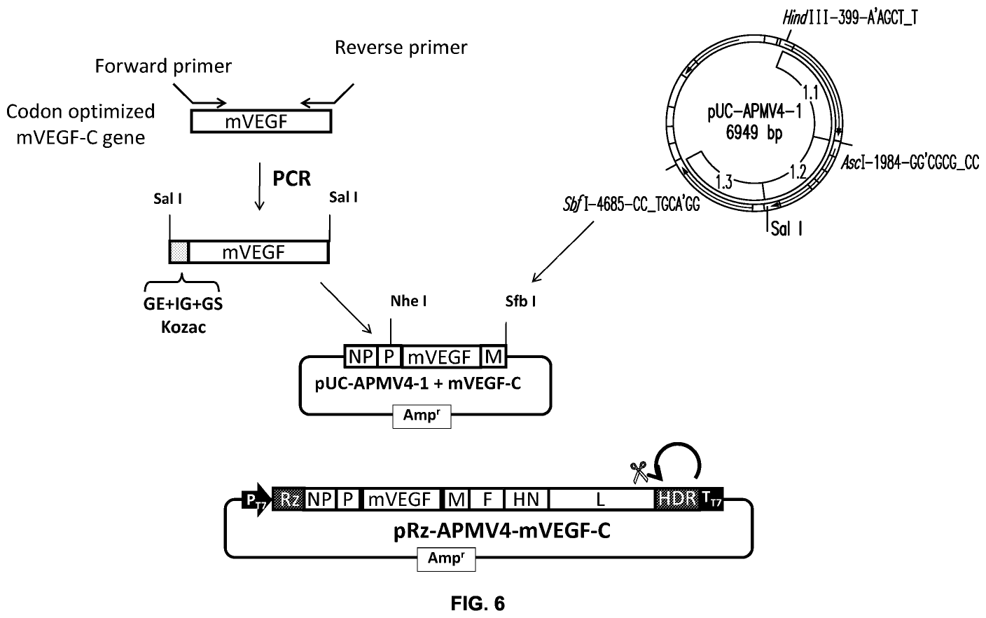

The size of the amplified insert was compliant with the rule of 6 to warrant efficient encapsidation of the genome by the viral NP. The PCR product was cloned into the Sal I site of the pRz-APMV4 plasmid. Because the Sal I site is not unique, the cloning was done in 2 steps: 1) the PCR product was cloned in the unique Sal I site of plasmid pUC-APMV4-1; and 2) A Nhe I-Sbf I restriction fragment (containing the mVEGF-C gene) was subcloned into plasmid pRz-APMV4. See, e.g., SEQ ID NO: 90 for plasmid pRz-APMV4 sequence. After confirmation of the presence of mVEGF-C, the plasmid is used to rescue a rAPMV4 expressing mVEGF-C as described above. Presence of the additional gene in the viral genome is confirmed by RT-PCR and expression of mVEGF-C is confirmed by immunological assays (ELISA and immune-fluorescence) on infected Vero cells. A schematic showing the protocol for the construction of the rescue plasmid pRz-APMV4-mVEGF-C is shown in FIG. 6.

### 6.2 Example 2: Oncolytic Activity of APMVs in B16-F10 and B16-VEGF-C+ Syngeneic Murine Melanoma Tumor Models

Tumor Growth Curves and Long-Term Survival

B16-F10 or B16-VEGF-C+ cells were implanted in the flank of the right posterior leg of C57BL/6 mice. Starting once the primary tumor reached a volume of 50 mm³ (about day 12 post-implantation of B16-F10 or B16-VEGF-C+ cells), the animals were intratumorally treated every other day (days 12, 14, 16, and 18) with a total of four doses of 10⁷ PFU of LS-L289A, 10⁷ PFU of APMV-4, or 50 μl of PBS for control mice. Tumor volume was monitored every 48 hours or every 24 hours when approaching the experimental end point of a diameter of 1 cm (≥500 mm³), after which mice were euthanized. Body weight was monitored every 48 hours.

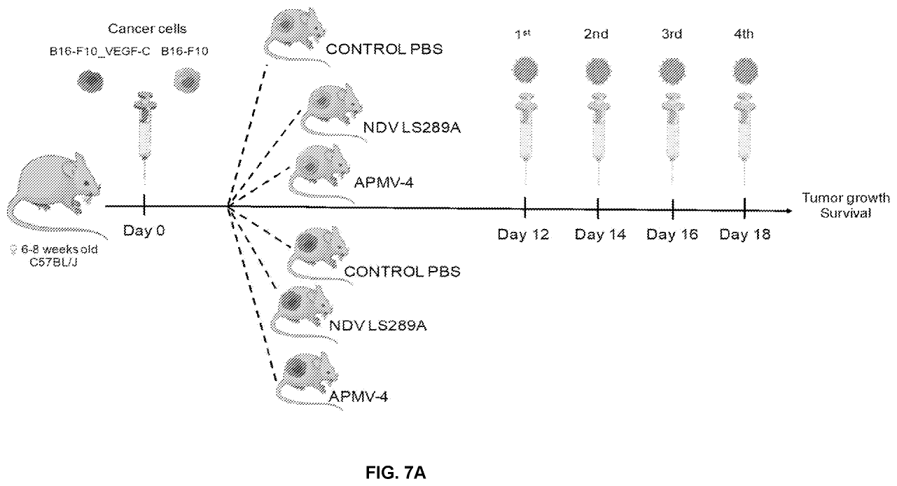

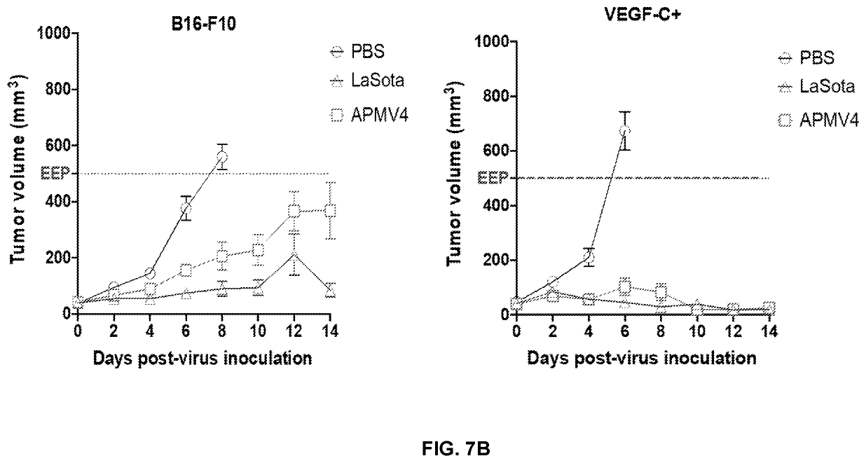

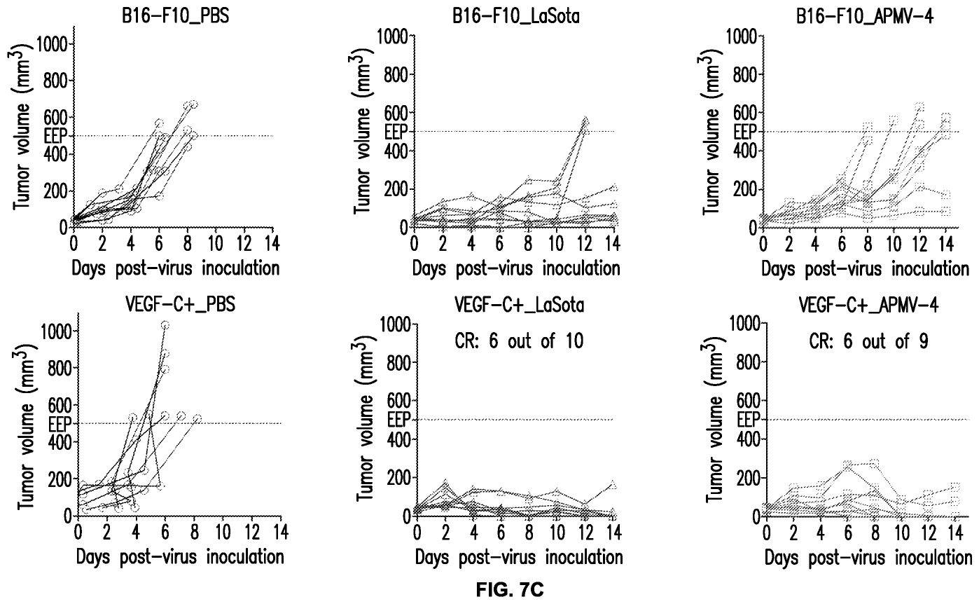

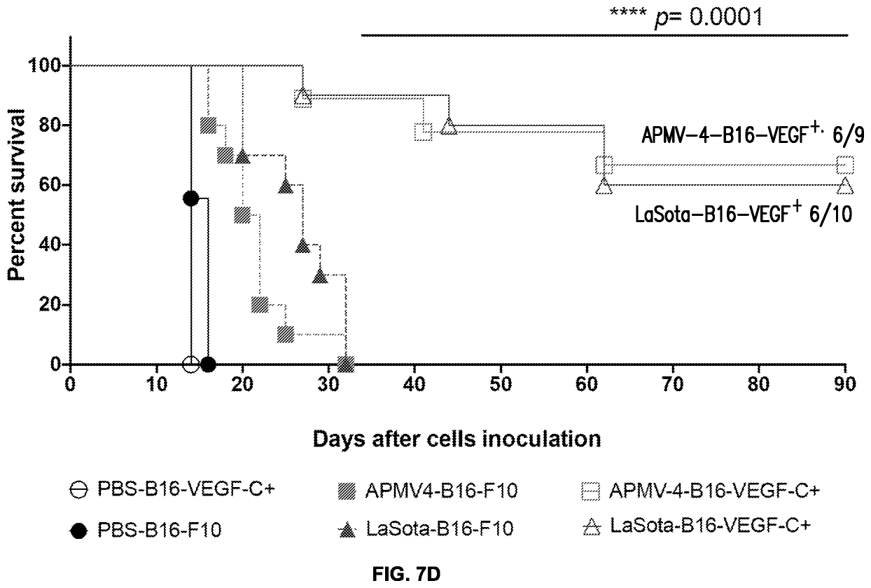

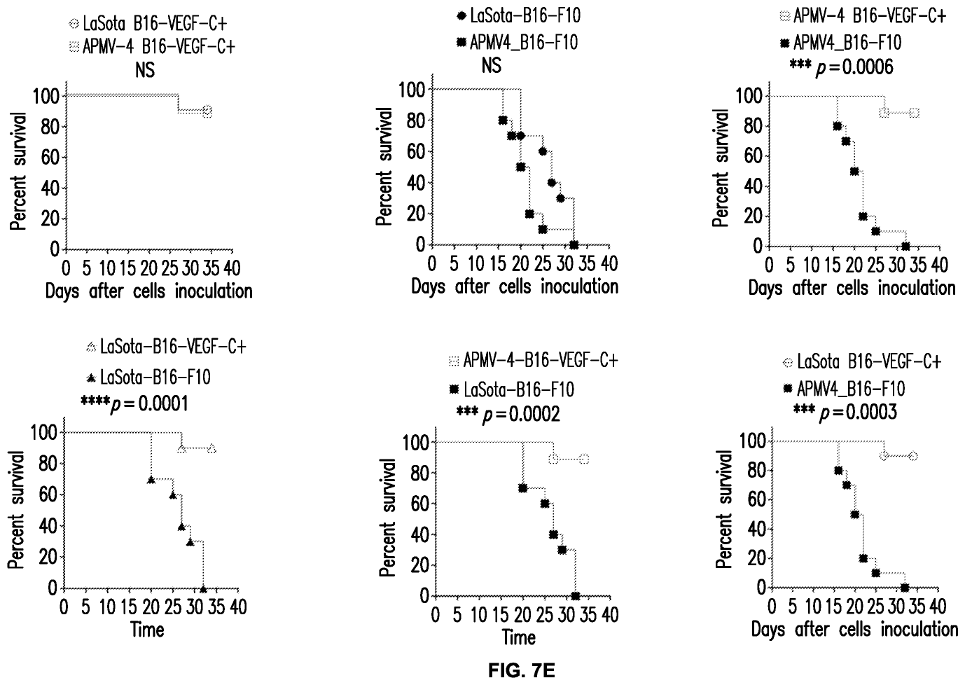

FIG. 7A shows a schematic of the experimental set up for Study 1. An analysis of tumor growth rate is shown in FIG. 7B (points represent average of tumor volume per experimental group at the indicated time point; error bars correspond to standard deviation of each group) and FIG. 7C (individual tumor growth curves; each point represents tumor volume per mice at the indicated time point). Data showing overall survival and a, comparative analysis between experimental groups of treated B16-F10 or B16-VEGF-C+ tumor-bearing mice are shown in FIGS. 7D an 7E, respectively.

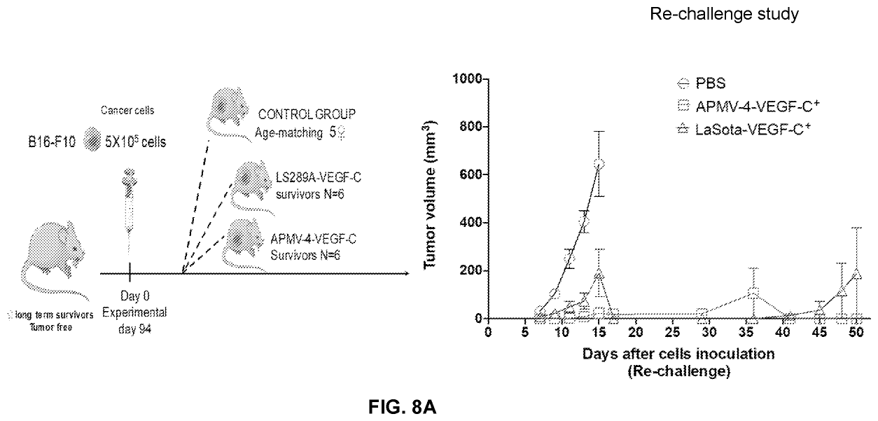

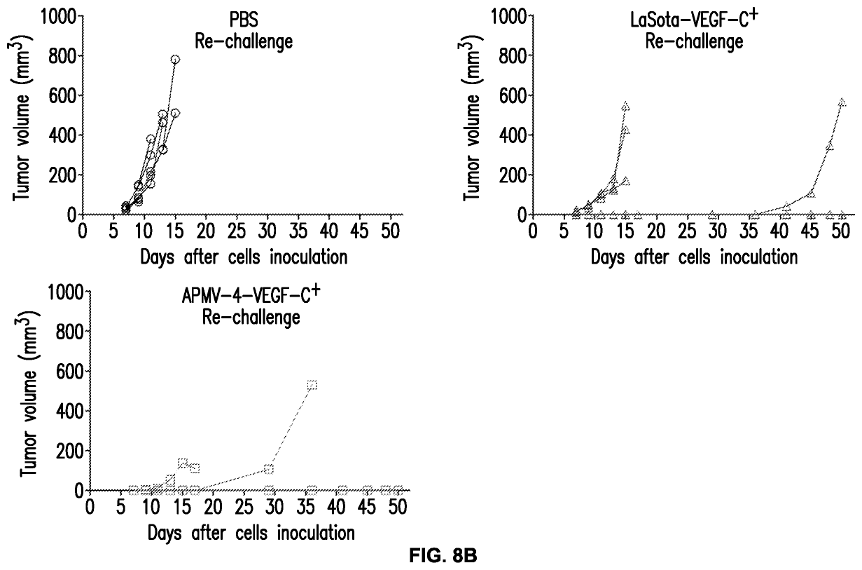

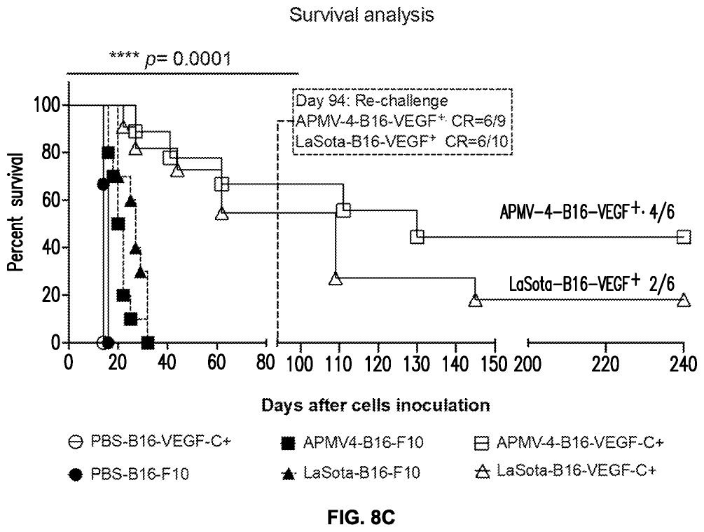

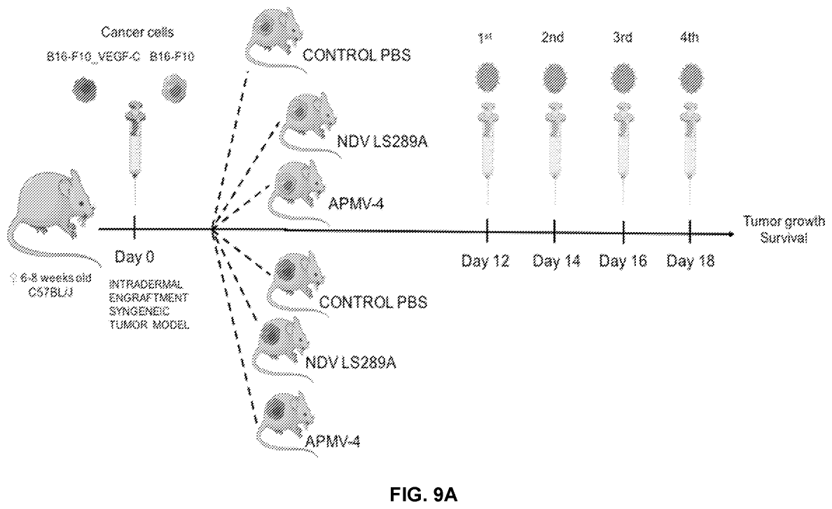

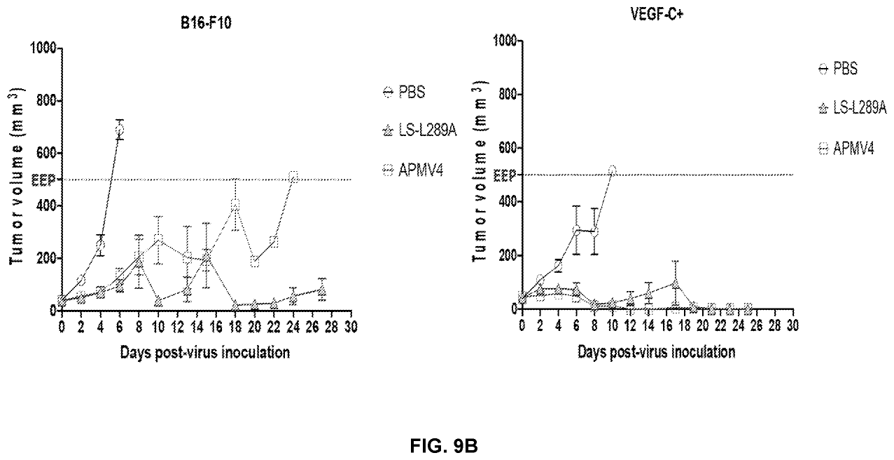

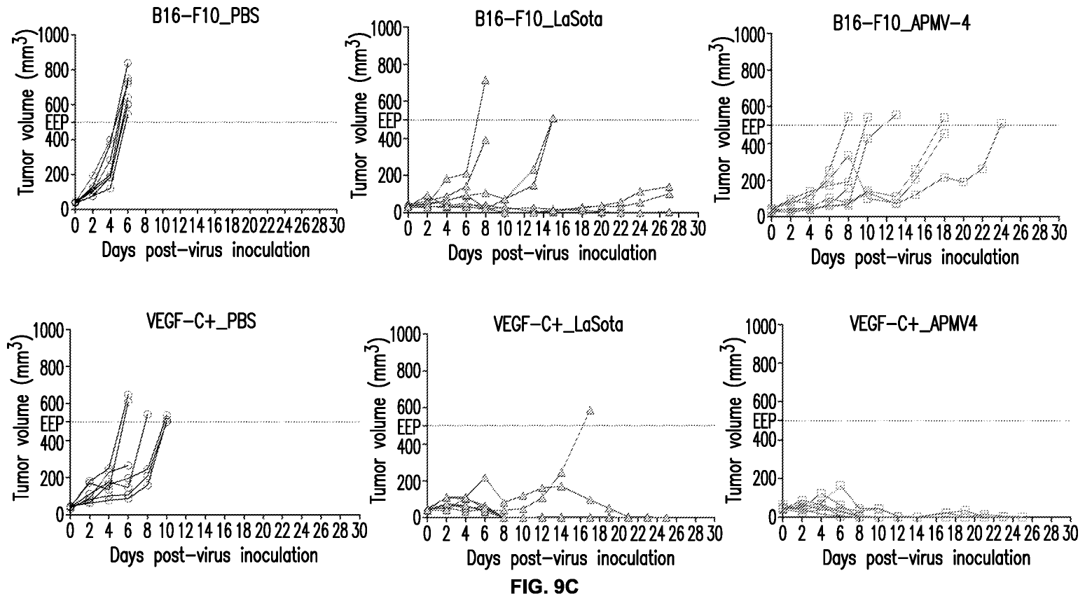

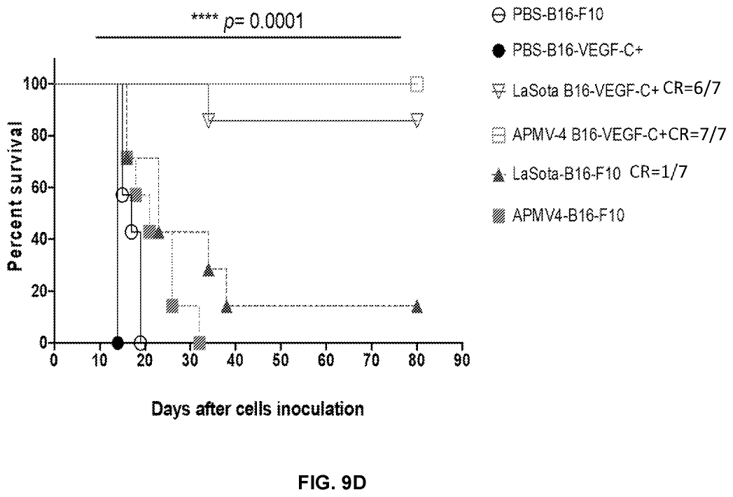

FIG. 9A shows a schematic of the experimental set up for Study 2. An analysis of tumor growth rate is shown in FIG. 9B (points represent average of tumor volume per experimental group at the indicated time point, error bars correspond to SD of each group) and FIG. 9C (individual tumor growth curves, each point represents tumor volume per mice at the indicated time point). FIG. 8D shows an overall survival analysis pre-re-challenge.

Re-Challenge

Long term survivors displaying complete remission (CR) of the primary tumor (day 94) were intradermally implanted with 3×10⁵ B16-F10 in the flank of the contralateral leg. As for the primary lesion, tumor volume and body weight loss were monitored every 48 hours or every 24 hours when the last volume estimation was approaching the experimental endpoint of a diameter of 1 cm (≥500 mm³).

FIG. 8A shows a schematic of the re-challenge experimental set up for the Study 1 (right panel) and an analysis of tumor growth rate (left panel). Points represent average of tumor volume per experimental group at the indicated time point. Error bars correspond to standard deviation of each group. FIG. 8B shows individual tumor growth curves. Each point represents tumor volume per mice at the indicated time point. FIG. 8C shows a post-re-challenge overall survival analysis of Study 1.

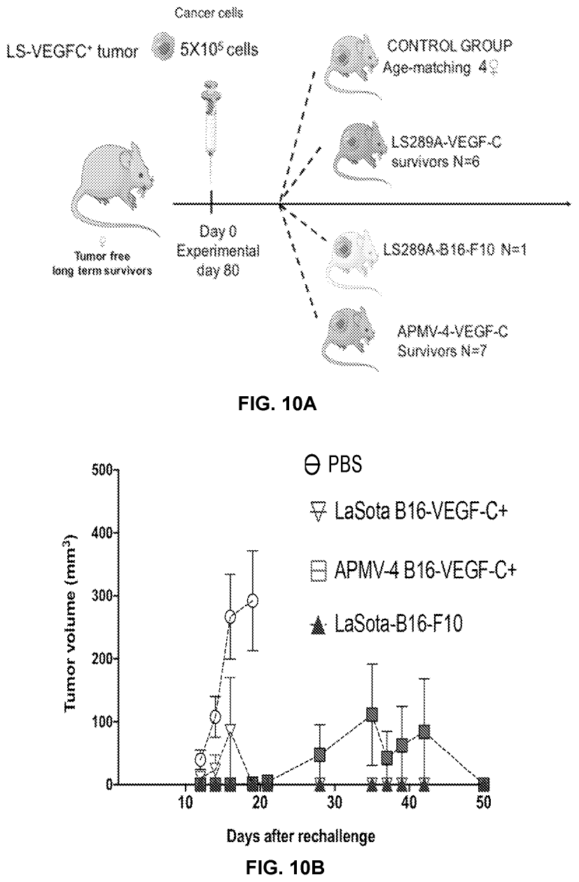

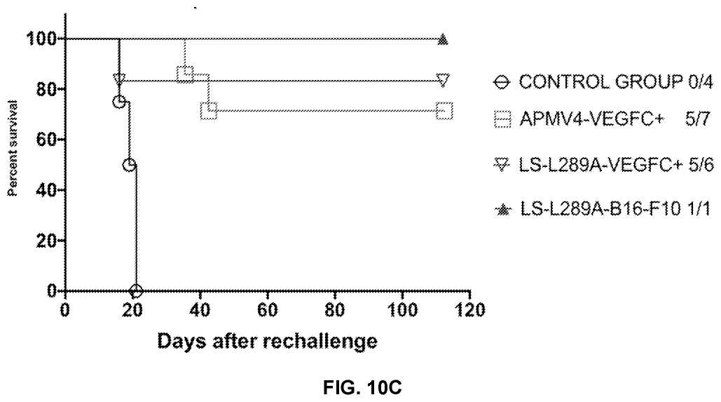

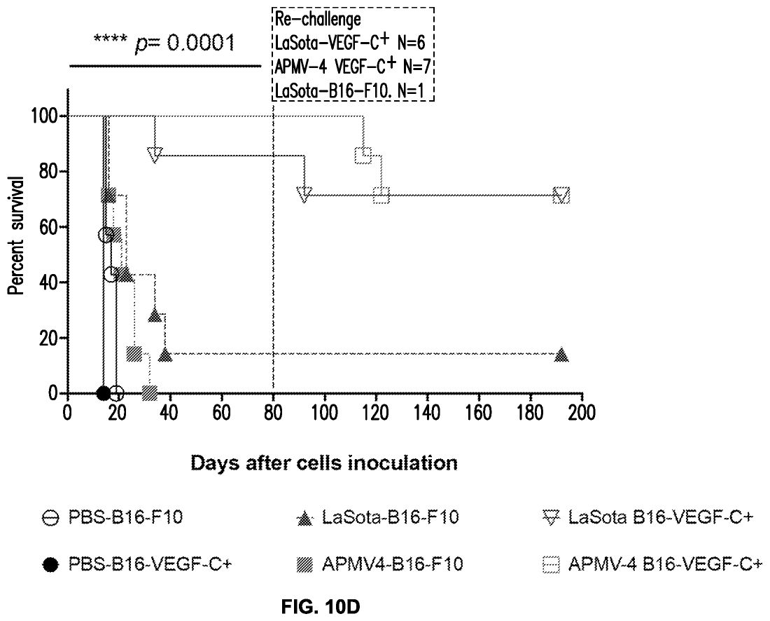

FIG. 10A shows a schematic of the re-challenge experimental set up for Study 2. An analysis of tumor growth rate is shown in FIG. 10B. Points represent average of tumor volume per experimental group at the indicated time point. Error bars correspond to standard deviation of each group. FIG. 10C shows survival post-re-challenge. FIG. 10D shows a survival analysis summary for Study 2.

The data demonstrates that the administration of NDV or APMV-4 to VEGF-C positive tumors increases the survival of animals and decreases the size of the tumors. In addition, the data demonstrates that re-challenge of animals that were administered NDV or APMV-4 and display complete remission of the primary tumors post-administration have reduced tumor volume and increased survival.

### 6.3 Example 3: VEGF-C Potentiates Anti-Tumor Immune Response Stimulated by the Viral dsRNA Mimic Poly(I:C)

Experimental Design

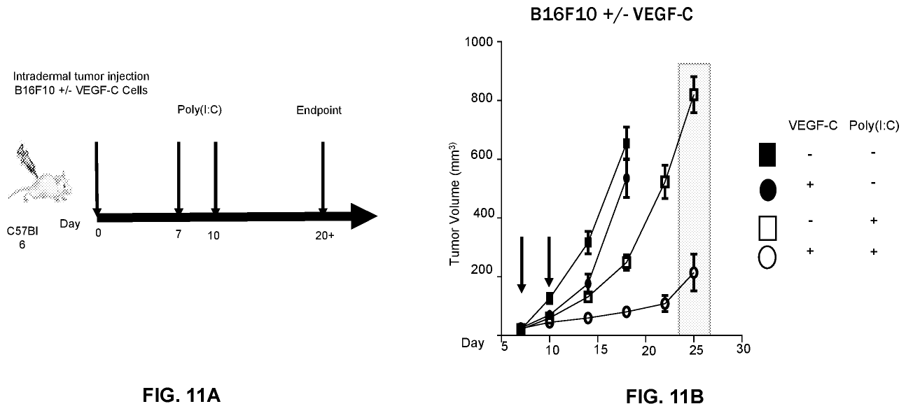

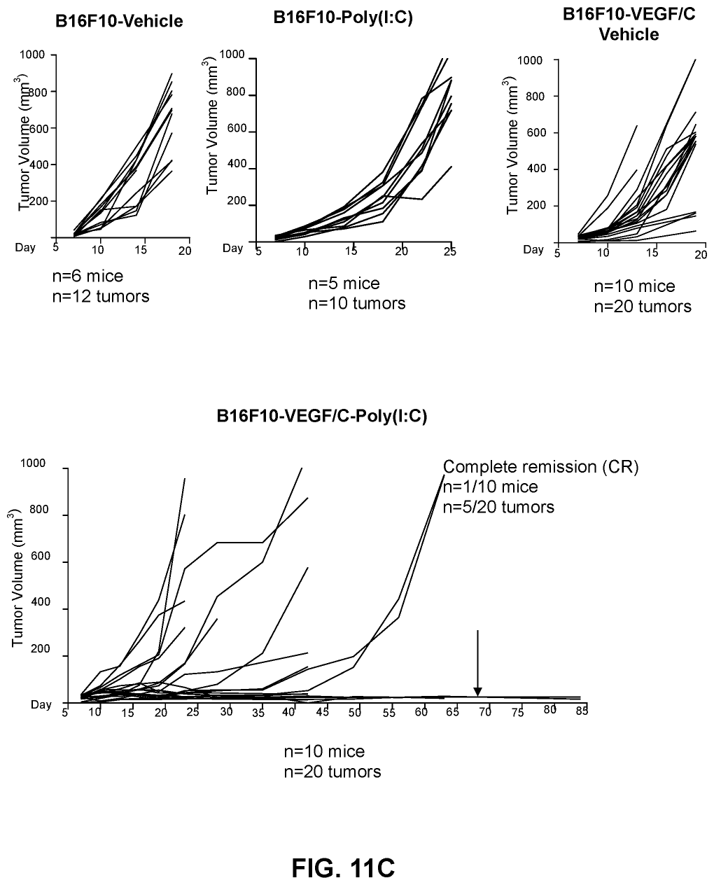

B16F10 or B16F10/VEGF-C+ cells (5×10⁵) were injected intradermally into C57BL6/J mice. Each mouse received two injections into the skin on lower back, left and right. On Day 7 (tumor volume 50 mm³) and 10 post tumor injection, 50 μg of Poly(I:C) or PBS control was injected intratumorally. Tumor volume was monitored by caliper and mice were euthanized when tumor diameter reached 10 mm on any axis. FIG. 11A shows a schematic of the study design.

Results

Data shown in FIGS. 11B and 11C shows tumor growth upon stimulation in B16F10 with Poly(I:C) with or without VEGF-C, or with a combination of both VEGFC and Poly(I:C), as indicated. FIG. 11B shows average tumor volume per experimental group+/−standard deviation, individual tumor growth curves are shown in FIG. 11C.

### 6.4 Example 4: NDV-VEGF-C Constructs

This example demonstrates that recombinant NDV-VEGF-C constructs expressing the full length VEGF-C are able to reduce tumor growth and extend survival of mice implanted with B16F10 tumors.

### 6.4.1 Methods

6.4.1.1 Recombinant NDV-VEGF-C

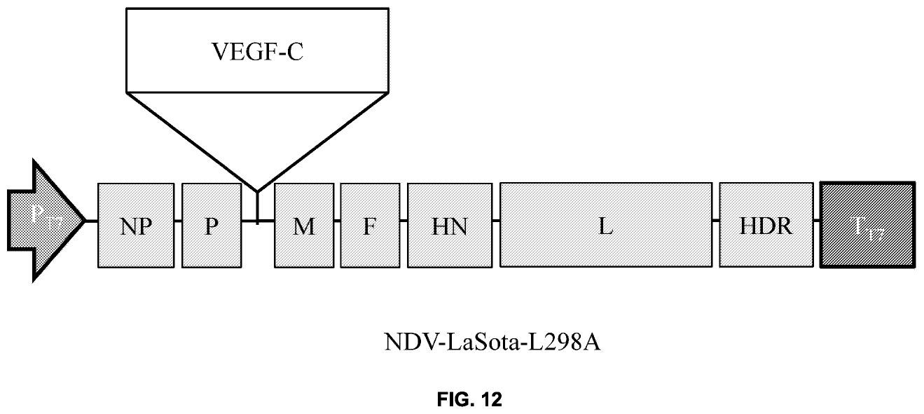

6 recombinant NDV-VEGF-C viral constructs were produced as described in Example 1, supra. Nucleotide sequences encoding the 6 VEGF-C constructs are disclosed in Table 5. As shown in FIG. 12, to generate each of the 6 recombinant NDV-VEGF-C viral constructs, the nucleotide sequence of VEGF-C construct was inserted between P and M transcription units of the cDNA sequence of the NDV LaSota strain genome.

6.4.1.2 Immunofluorescent Staining of Cells In Vitro.

Wells of glass slides were seeded with 5×10⁴ Vero cells and Vero cells were infected with different dilutions of the NDV viral constructs (“NDV-VEGF-C”). After 12 hr, cells were washed with PBS and fixed in 10% formalin for 10 minutes at room temperature (RT). Cells were rinsed with PBS at RT for 5 min. Fixed cells were blocked with 1% goat serum in PBS-Tween 20 (0.5%) for 1 h at RT. Cells were then incubated with anti-VEGFC primary antibody (R&D, AF752,) diluted 1:100 in 1% goat serum-PBS-Tween 20 0.5% at RT for 1 hr. Secondary anti-goat Alexa-594 antibody (Jackson Immunoresearch, 705-586-147) was diluted 1:500 in 1% goat serum PBS-Tween 20 0.5% and the cells were incubated with secondary antibody at RT for 1 h.

6.4.1.3 ELISA

293T cells were transfected with the different constructs and incubated with serum-free cell culture media for 24 hours. After 24 h, the media was collected, centrifuged and filtered. 50 μl of the conditioned media were analyzed by ELISA following the manufacturer instructions (R&D, cat. DVE00).

6.4.1.4 Western Blot Analysis.

293T cells were transfected with the different viral constructs and supernatants were collected after 24 hr. 8 ml of each sample was loaded on an Amicon Ultra 15 3k filter (Cat 900324). Samples were centrifuged at 4000 g for 1 h at 4° C. and the volume of all samples was adjusted to 310 μl. 20 μl of each concentrated sample were mixed with 10 μl of 6× Laemmli buffer, heated at 96° C. for 5 minutes, and loaded onto 15% agarose gel. Western blotting was performed using chemiluminescence.

6.4.1.5 Mouse Tumor Studies.

C57BL mice were injected with 5×10⁵ B16F10 cells, 8-10 mice per group. Tumors were allowed to grow and virus treatment was started when tumors reached 5 mm. PBS (control group) or 1×10⁷ viral PFU of NDV or NDV/VEGF-C were administered to the mice intratumorally in 100 μl of PBS every 2 days. A total of 4 injections were administered to each mouse. Tumor volume was measured every two days.

6.4.1.6 Immunohistochemistry.

Immunohistochemistry was performed on paraffin-embedded tissue sections using the Leica Bond RX automated immunostainer (Leica Biosystems), according to the Leica staining protocol. Briefly, all slides were deparaffinized using a heated Bond™ Dewax Solution (Leica cat. no. AR9222) and washed with 1× Bond™ Wash Solution (Leica cat. no. AR9590). Epitope retrievals were carried out for 20 minutes using the citrate-based Bond™ Epitope Retrieval 1 solution (Leica cat. no. AR9961). Slides were then blocked with a 3-4% v/v hydrogen peroxide block for five minutes that is included in the Bond™ Polymer Refine Detection kit (Leica cat. no. DS9800) and used for DAB staining. All antibodies were diluted using Bond™ Primary Antibody Diluent (Leica Biosystems AR9352). The following antibodies and dilutions were used: VEGF-C (R&D, cat. number: AR752), LYVE-1 (Angiobio, Cat. number: 11-033) 1:100, CD8 (Biolegend, Cat. number: 100701), 1:300, goat anti-rat HRP (ThermoScientific, cat. number 31470) 1:1000, donkey anti-goat HRP (Jackson Immunoresearch, cat. number 705036147).

### 6.4.2 Results

6.4.2.1 NDV/VEGF-C Engineered Constructs with Six VEGF-C Variants

We generated six different NDV constructs by inserting VEGF-C between P (phosphoprotein) and M (matrix protein) regions of NDV (FIG. 12). The resulting NDV/VEGF-C constructs comprised one of three full length VEGF-C variants one of three mature VEGF-C variants (fully proteolytically processed forms) with mutations generated to improve binding affinity to its receptor and/or stability (Joukov, 1997, “Proteolytic processing regulates receptor specificity and activity of VEGF-C.” EMBO J. 116(13):3898-911; Anisimov, 2009, “Activated forms of VEGF-C and VEGF-D provide improved vascular function in skeletal muscle.” Circ Res. 104(11):1302-12; Jeltsch, 2014, “CCBE1 enhances lymphangiogenesis via A disintegrin and metalloprotease with thrombospondin motifs-3-mediated vascular endothelial growth factor-C activation.” Circulation. 129(19):1962-71). Details regarding the VEGF-C variants are provided in Table 5.

TABLE 5
VEGF-C Construct

| | Sequence | | Short |
|---|---|---|---|
| Construct full name | number | Full designation | designation |
| mouse VEGF- C full length wt | 7 | SN1 VEGF-C FL | FL - WT |
| codon optimized | | WT | |
| mouse VEGF- C full length | 9 | SN2 VEGF-C FL | FL - 133A |
| Cys133Ala mutation codon | | 133A | |
| optimized | | | |
| mouse VEGF- C full length | 8 | SN3 VEGF-C FL | FL - 152S |
| Cys152Ser mutation codon | | 152S | |
| optimized | | | |
| mouse VEGF- C mature | 10 | SN4 VEGF-C | dNdC - WT |
| (dNdC) wt codon optimized | | DNDC WT | |
| mouse VEGF- C mature | 12 | SN5 VEGF-C | dNdC - 133A |
| (dNdC) Cys133Ala mutation | | DNDC 133A | |
| codon optimized | | | |
| mouse VEGF- C mature | 11 | SN6 VEGF-C | dNdC - 152S |
| (dNdC) Cys152Ser mutation | | DNDC 152S | |
| codon optimized | | | |

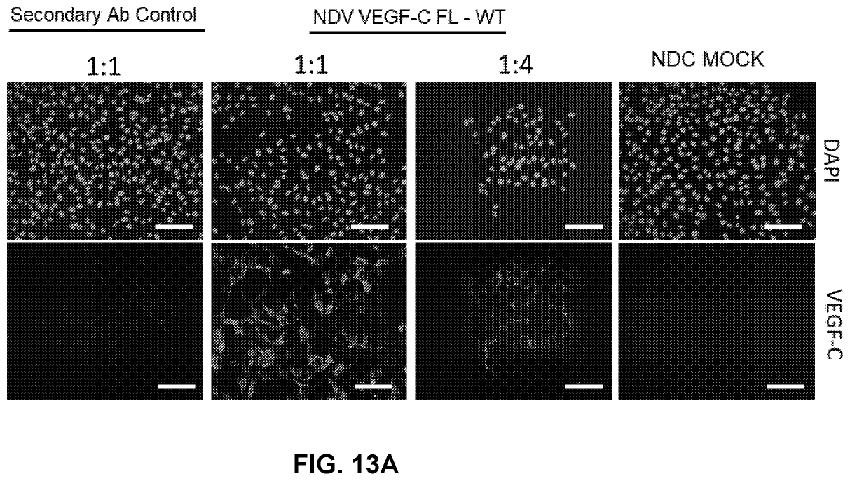

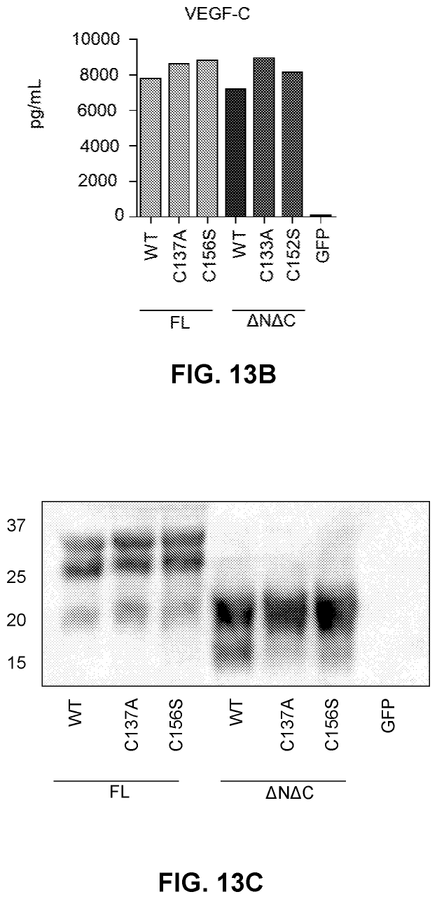

Transduction of Vero cells with NDV-VEGF-C FL-WT resulted in high expression levels of VEGF-C by these cells in vitro (FIG. 13A). High levels of VEGF-C protein (˜7-9 ng/ml) were detected by ELISA in supernatants of 293T cells transfected with each of the six different VEGF-C variants (FIG. 13B). Furthermore, Western Blot analysis showed the expected pattern of proteolytically processed forms of VEGF-C for each construct (FIG. 13C). VEGF-C is synthesized as a precursor in which the central VEGF homology domain (VHD) is flanked by N- and C-terminal pro-peptides. Proteolytic removal of the pro-peptides increases VEGF-C affinity for VEGFR-3, and the resulting mature protein can also activate the major angiogenic receptor VEGFR-2 (Joukov et al., 1997 EMBO J. 116(13):3898-911; Jeltsch et al., Circulation. 2014 May 13; 129(19):1962-71)). Western blot analysis demonstrated that each of the three constructs comprising a full length form of VEGF-C produced mainly intermediately processed form of VEGF-C (˜33 kDa) (FIG. 13C) which is believed to have high affinity for VEGFR-3. In contrast, each of the three constructs comprising a mature form of VEGF-C (ΔNΔC) produced mainly 21 kDa protein (FIG. 13C) which binds VEGFR-2 in addition to VEGFR-3. Together, these data demonstrate that different VEGF-C variants engineered into NDV are expressed correctly and at high levels by cells transduced with the NDV containing the VEGF-C construct in vitro.

6.4.3 Effects of Treatments with NDV/VEGF-C on Tumor Growth and Survival

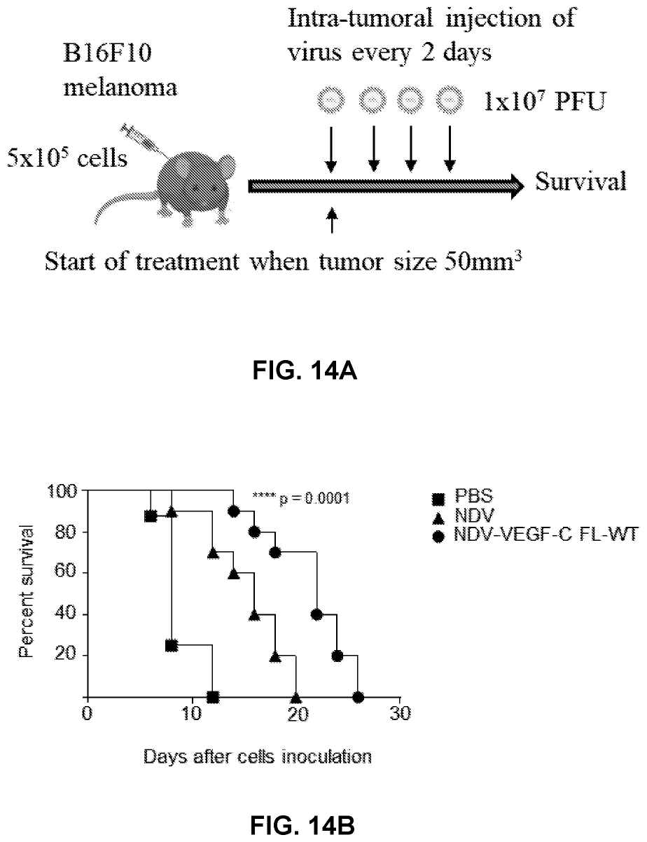

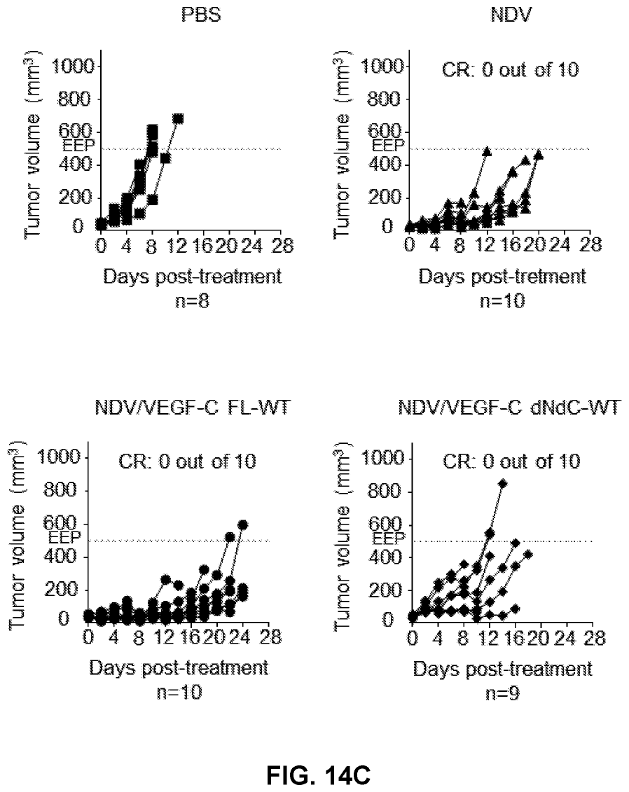

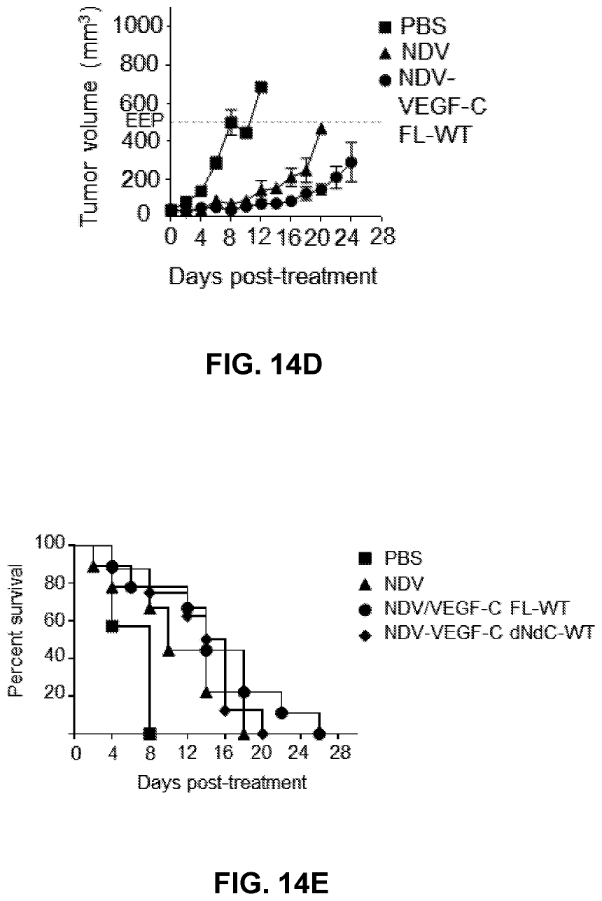

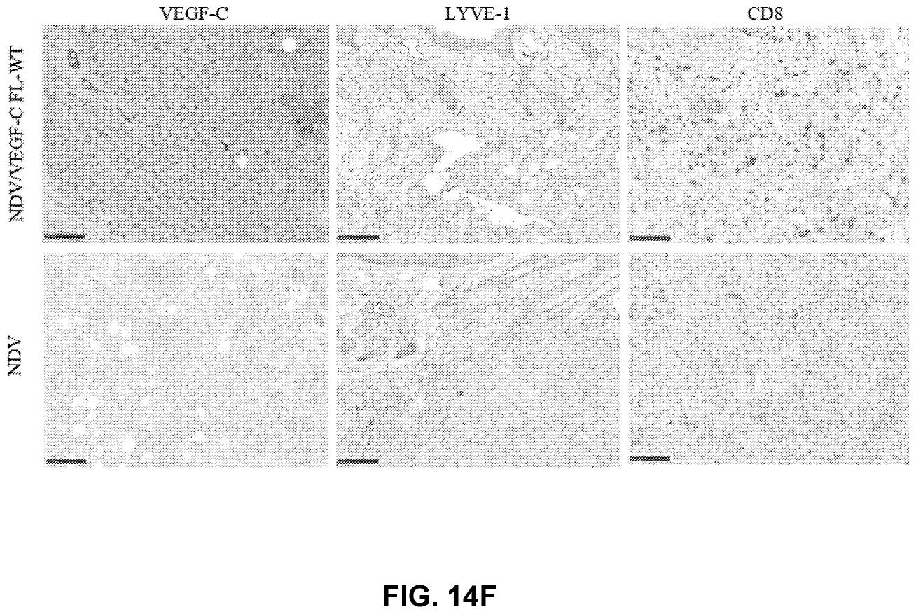

The efficacy of engineered NDV-VEGF-C wild-type (“wt”) constructs on inhibition of tumor growth was evaluated in mice. B16F10 tumors were treated by four intra-tumoral injections of NDV-VEGF-C and monitored for tumor growth and survival (FIG. 14A). Treatment with NDV-VEGF-C construct expressing full length, wild-type (wt) VEGF-C significantly extended survival of treated mice (FIG. 14B). Monitoring of tumor growth showed delayed tumor growth in mice treated with NDV-VEGF-C FL-WT, but not with NDV-VEGF-C dNdC-WT (FIGS. 14C and 14D). In accordance with these data, treatment with NDV-VEGF-C FL-WT extended life-span of animals, whereas treatment with NDV-VEGF-C dNdC-WT did not (FIG. 14E). Immunostaining of tumors with an anti-VEGF-C antibody resulted in a strong signal, indicating that high levels of VEGF-C protein are produced in tumors upon administration of NDV-VEGF-C FL-WT (FIG. 14F). Furthermore, immunostaining for lymphatic vessels using an anti-LYVE-1 antibody showed enlarged lymphatics and increased densities of lymphatics in NDV/VEGF-C treated tumors, indicating that functional VEGF-C has been produced (FIG. 14F). Finally, NDV-VEGF-C treatment lead to a striking increase of CD8+ T-cell densities in tumors, that were uniformly distributed throughout (FIG. 14F). Taken together, these data demonstrate that the NDV-VEGF-C wild type full length construct (WT-FL) effectively reduces tumor growth and extends survival, whereas NDV-VEGF-C dNdC-WT variant does not. These data further indicate that the anti-tumor effects of VEGF-C are likely mediated through VEGFR-3 and not through VEGFR-2.

### 6.5 Example 5: In Vivo Effects of NDV/VEGF-C

6.5.1 methods

6.5.1.1 Mouse Experiments.

B16F10 or B16F10-VEGF-C tumor cells (5×10⁵ cells in 100 μl serum-free media) were injected intradermally into the right flank of six to eight-week-old mice (Jackson, C57B1/6J, cat. 000664). Mouse weights and tumor sizes were measured every two days. Treatment was started when tumors reached 5 mm in size. 50 μL of a solution containing PBS or NDV (10⁷PFUs/dose) were administered to the mice intratumorally every two days for a total of 4 treatments. Mice were monitored until humane endpoint. Mouse experiments were performed in accordance with protocols approved by the Institutional Animal Care and Use Committee (IACUC).

6.5.1.2 Flow Cytometry.

Flow cytometry was done using Aurora Spectral Cytometer (Cytek Biosciences) or LSR Fortessa X-20 (BD Biosciences). In brief, tissues were dissected and minced in a sterile petri dish in ice cold PBS (Invitrogen). Tumor tissues were dissociated with Mouse Tumor Dissociation Kit (Miltenyi) enzymes in Octomacs Dissociator with Heaters (Miltenyi). Lymph nodes were dissociated with Collagenase D enzyme (1 mg/ml, Roche) in a 37° C. water bath for 1 hour. Dissociation reactions were stopped with the addition of ice-cold FACS buffer (1% FBS, 0.09% NaN3 in PBS). Erythrocytes were lysed using RBC lysis (eBioscience) for 1 minute on ice. Lysis was stopped by the addition of ice-cold FACS buffer. Dissociated tissues were pressed through a 70 μM nylon filter to create a single cell suspension. Cell yield and viability were determined using Countess II Automated Cell Counter (ThermoFisher). Samples were stained with primary antibodies (see Table 6 below) targeting cell surface markers for 30 minutes on ice (1.0×10⁶ cells/100 μL). Cells were then fixed and permeabilized with FOXP3 Transcription Factor Staining Buffer Set (eBioscience). Samples were then stained with primary antibodies targeting intracellular markers. Compensation and reference groups were calculated using UltraComp beads (eBioscience). Dead cells were excluded using LIVE/DEAD Fixable Yellow Dead Cell Stain Kit (Molecular Probes). 2.0×10⁴-1.0×10⁵ viable CD45+ cells were acquired/sample. Flow Cytometry Data Analysis was done in FCS Express Version 7.0 and Cytobank. tSNE analysis was done in FCS Express with 500 iterations, Barnes-Hut Approximation of 0.5, and perplexity of 0.3. SPADE analysis was done in Cytobank

TABLE 6
Antibodies used for Spectral and Flow Cytometry

| | | | Concentration (μg |
|---|---|---|---|
| Antibody | Fluorochrome | Clone | antibody/1.0 × 10⁶ cells) |
| F4/80 | BV-421 | Biolegend (BM8) | 0.1 |
| CD80 | V450 | BD Biosciences (16- | 0.1 |
| | | 10A1) | |
| CD45 | BV480 | Biolegend (30-F11) | 0.08 |
| CD83 | BV650 | Biolegend (Michel-19) | 0.1 |
| PD-L1 | BV711 | BD Biosciences | 0.1 |
| | | (M1H5) | |
| MHCII | BV785 | Biolegend | 0.08 |
| | | (M5/114.15.2) | |
| CD86 | FITC | Biolegend (GL-1) | 0.1 |
| CD11C | PerCP | Biolegend (N418) | 0.2 |
| CD64 | PerCPCy5.5 | Biolegend (X54-5/7.1) | 0.2 |
| CD103 | PE | Biolegend (2E7) | 0.1 |
| B220 | PECF594 | BD Biosciences (RA3- | 0.07 |
| | | 6B2) | |
| Ly6G | Pe-Cy7 | Biolegend (1A8) | 0.08 |
| CD11B | APC | Biolegend (M1/70) | 0.08 |
| CD49B | AF647 | Biolegend (HMα2) | 0.1 |
| Ly6C | AF700 | Biolegend (HK1.1) | 0.07 |
| CCR2 | APCFire750 | Biolegend | 0.1 |
| | | (SA203G11) | |
| CD62L | BV421 | Biolegend (MEL-14) | 0.1 |
| KLRG1 | V450 | BD Biosciences (2F1) | 0.1 |
| CD8 | BV510 | Biolegend (53-6.7) | 0.1 |
| CD4 | PerCPEF710 | ThermoFisher (GK1.5) | 0.08 |
| CD3 | BV750 | BD Biosciences (SK7) | 0.1 |
| CXCR3 | BV650 | BD Biosciences | 0.1 |
| | | (CXCR3-173) | |
| CD28 | AF488 | Biolegend (E18) | 0.1 |
| CD25 | APC | Biolegend (PC61) | 0.2 |
| CTLA4 | APCR700 | BD Biosciences | 0.1 |
| | | (UC10-4F10-11) | |
| CD44 | APCFire750 | Biolegend (IM7) | 0.1 |
| FOXP3 | V450 | BD Biosciences | 0.2 |
| | | (MF23) | |
| NK1.1 | BV650 | Biolegend (PK136) | 0.1 |
| IFNg | BV711 | Biolegend (XMG1.2) | 0.1 |
| TNFα | BB700 | BD Biosciences (MP6- | 0.1 |
| | | XT22) | |
| GrB | FITC | Biolegend (GB11) | 0.1 |
| PD1 | BV605 | Biolegend (29F.1A12) | 0.1 |

6.5.1.3 Immunofluorescent Staining

Immunofluorescent staining was performed on fresh-frozen acetone/methanol fixed tissue sections. Briefly, all slides were fixed in cold acetone for 5 minutes followed by 2 minutes in cold 80% methanol. All primary antibodies were diluted in PBS-BSA 3% and incubated for 2 hours at room temperature. Secondary antibodies were incubated for 1 hour at room temperature. The following antibodies and dilutions were used: CD8 (Biolegend, cat. number: 100701) 1:300, CD4 (Biolegend, cat. number 100505) 1:300, CD11c (Biolegend, cat. number: 117301) 1:300, goat anti-rat AlexaFluor594 (Molecular Probes, cat. number: A11007), goat anti-armenian hamster FITC (Jackson Immunoresearch, cat. number: 127-095-160). For quantification of immunostaining images were acquired and quantified using NIS image software (Nikon).

### 6.5.2 Results:

6.5.2.1 NDV Treatment Leads to Complete Tumor Regression and Long-Term Survival of Mice with VEGF-C Expressing Melanomas

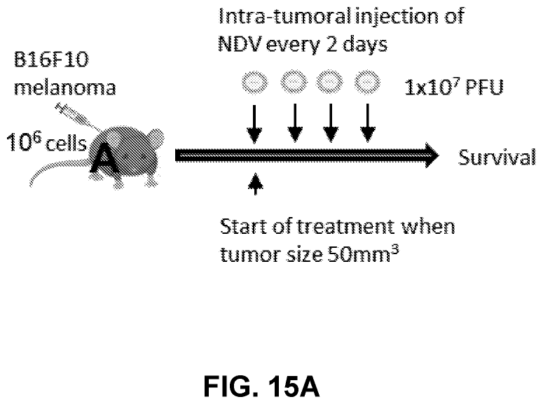

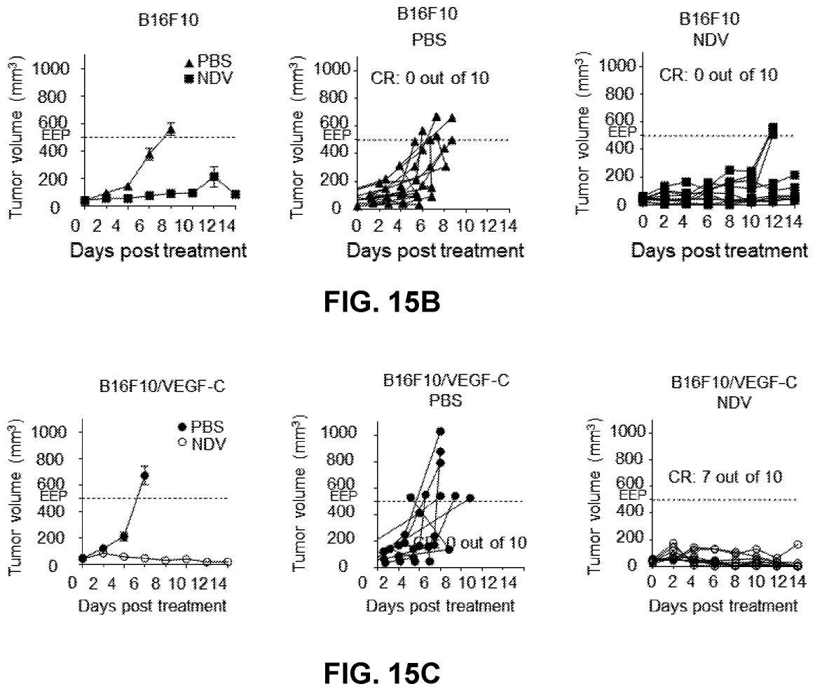

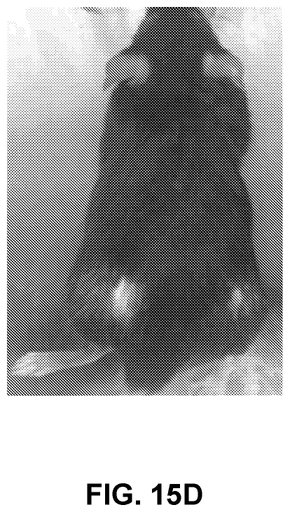

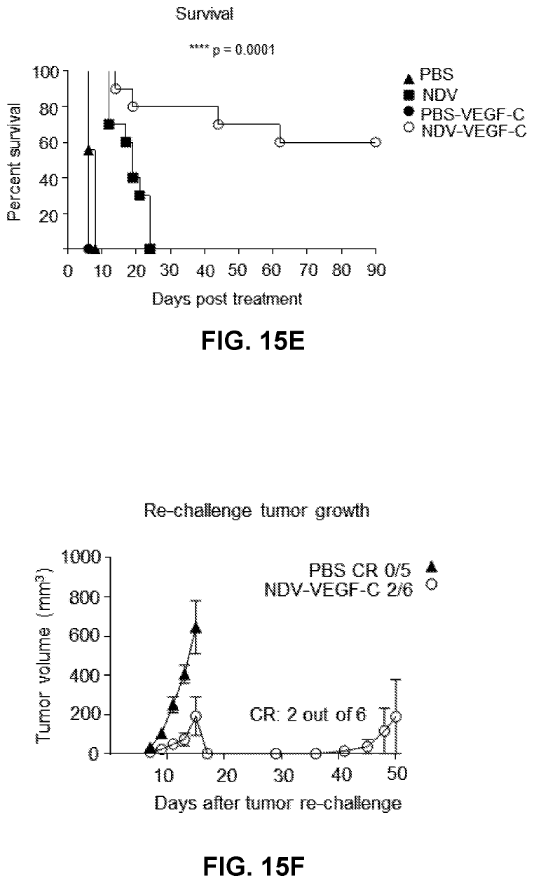

To examine the effects of NDV oncolytic viral therapy on tumors expressing VEGF-C, B16F10 or B16F10/VEGF-C mouse melanomas were treated with intra-tumoral injections of NDV every two days, for total of four treatments (FIG. 15A). NDV treatment of B16F10 tumors led to significant tumor growth reduction and extended life of mice, but eventually all mice developed large tumors and had to be sacrificed. In contrast, NDV treatment of B16F10 tumors expressing VEGF-C led to complete inhibition of tumor growth and eradication of tumors in 70% of the animals (Complete response—CR: B16F10-NDV, 0/10; B16F10/VEGF-C-NDV, 7/10) (FIGS. 15B and 15C). Mice whose tumors were eliminated showed vitiligo at the tumor site shortly after tumor rejection, and were recognizable as survivors based on the white patches of hair (FIG. 15D). Longitudinal analysis of animals in different treatment groups showed long-term survival only in mice that had VEGF-C expressing tumors treated with NDV (FIG. 15E). Survivor mice have remained healthy for over a year at this point (data not shown). Upon re-challenge with B16F10 tumor cells, 33% of mice were protected from developing tumors in NDV/VEGF-C group only (FIG. 15F). These data demonstrate that combination of NDV and VEGF-C in tumors has potent anti-tumor effect, leading to long-term survival after tumor eradication in majority of the animals.

6.5.2.2 Immunophenotyping Reveals Unique Immune Cell Subtypes in Tumors Expressing VEGF-C and Treated with NDV

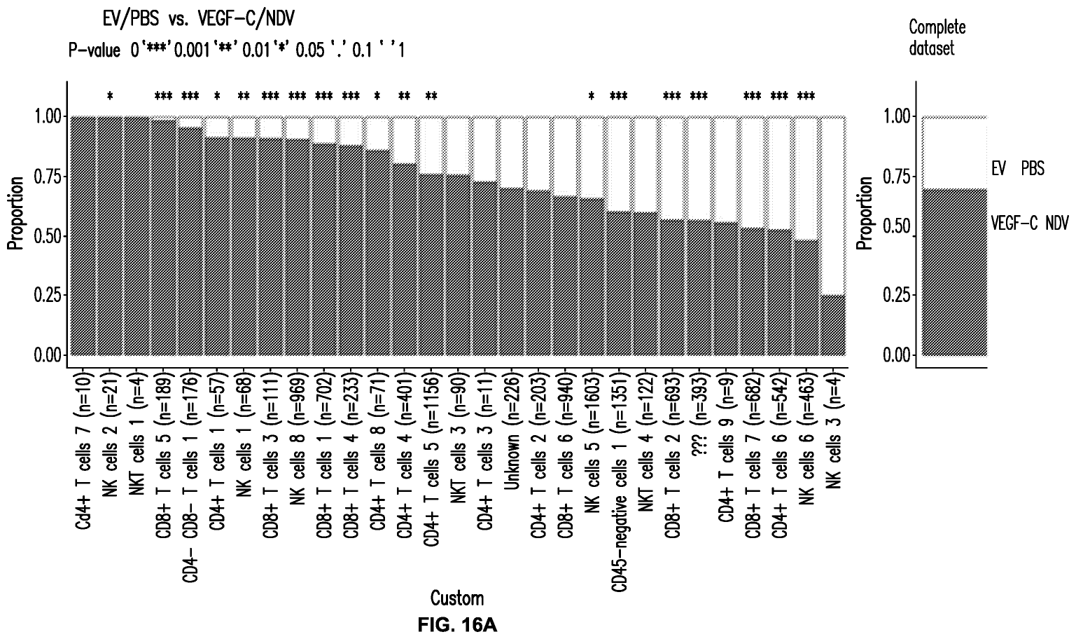

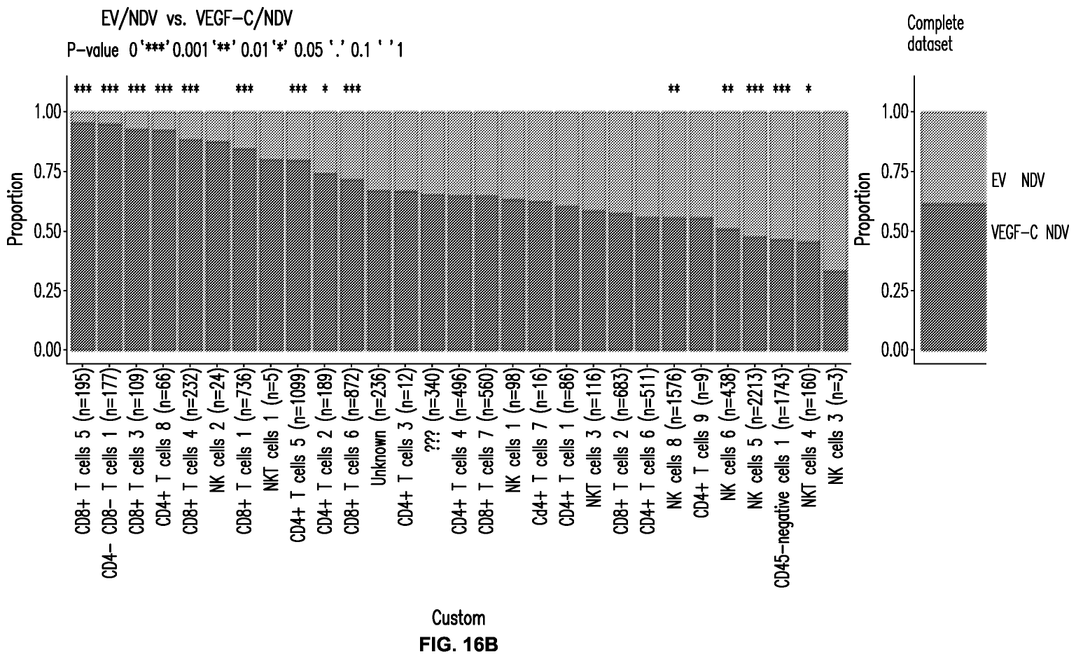

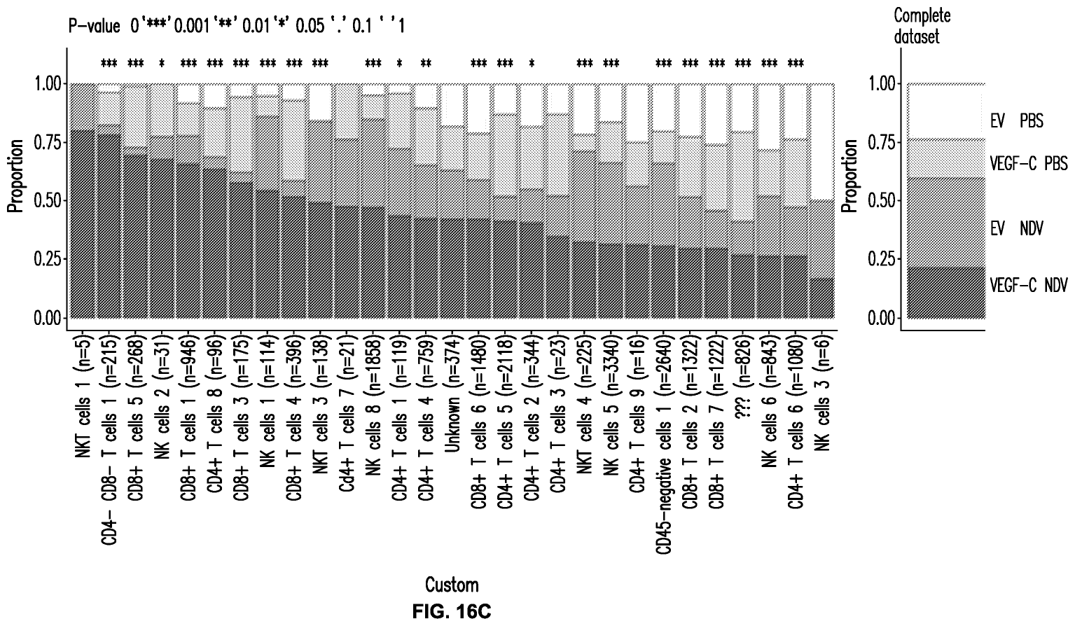

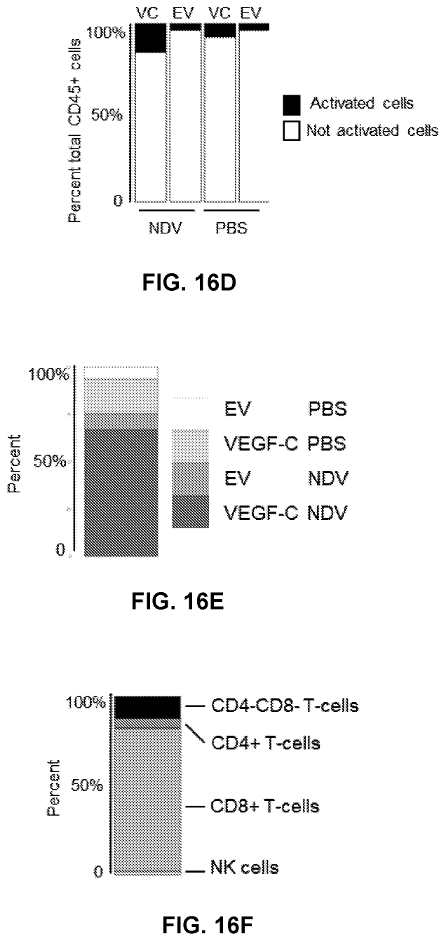

The underlying immunological basis of tumor rejection driven by NDV and VEGF-C was investigated by performing immunophenotyping of tumor immune cells using Aurora spectral flow cytometry. Analysis of immune cell subtypes in different treatment groups clearly revealed enriched and unique immune cell populations in NDV/VEGF-C tumors. An intracellular flow cytometry panel revealed multiple subsets of CD8+, T-cells, CD4+ T-cells and NK cells uniquely activated in NDV/VEGF-C tumors compared to control, PBS-treated B16F10 (FIG. 16A). NDV-treated B16F10/VEGF-C tumors were particularly enriched in activated CD8+ T-cells compared to NDV-treated tumors not expressing VEGF-C (FIG. 16B). Comparison of all four treatment groups clearly showed that several CD8+ T-cell subsets were predominantly seen in NDV/VEGF-C tumors (FIG. 16C). Among these, prominent subtypes included CD4-CD8− T-cells that expressed TNFα, CD4+ T cells expressing high levels of TNFα and IFNγ, as well as CD8+ T-cells expressing TNFα, IFNγ and GranzymeB. NK cells expressed Granzyme B, high levels of TNFα and dim levels of IFNγ. Comparison of all activated immune cells across treatment groups revealed very high levels of activated cells in NDV/VEGF-C group, with more than 16% of all immune cells in tumors being activated (FIGS. 16D and 16E). The main subset of activated cells was CD8+ T-cells, comprising more than 70% of all activated cells, followed by CD4⁻CD8⁻ T-cells, CD4+ T-cells and NK cells (FIG. 16F). Taken together, these data show high activation status of CD8, CD4 and NK cells driven by combination of VEGF-C and NDV effects in tumors. Immune cell activation status was greatly heightened with VEGF-C in comparison to NDV treatment alone.

6.5.2.3 Enrichment of T-Cells and Dendritic Cells Tumors Expressing VEGF-C and Treated with NDV is Associated with Expansion of Lymphatic Vessels

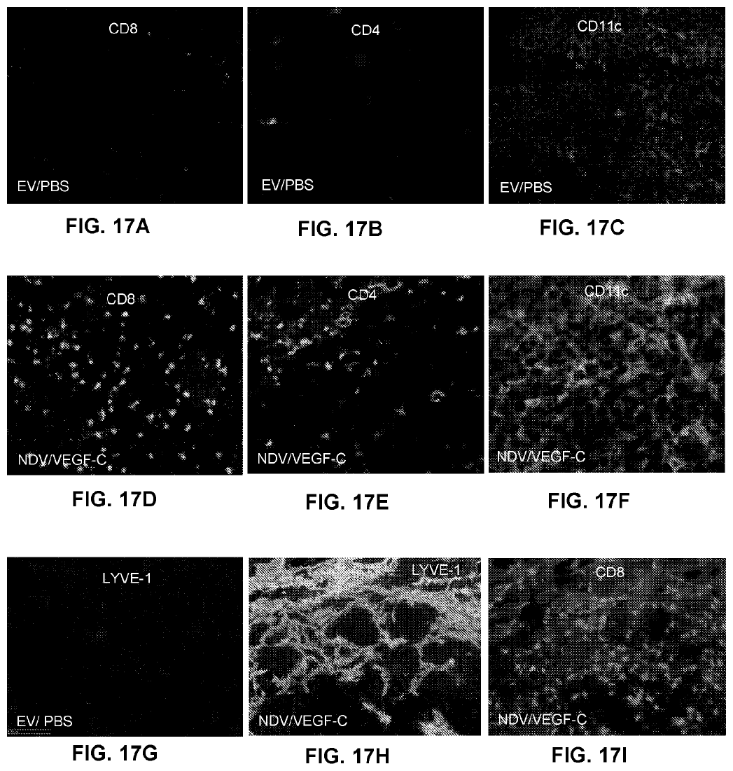

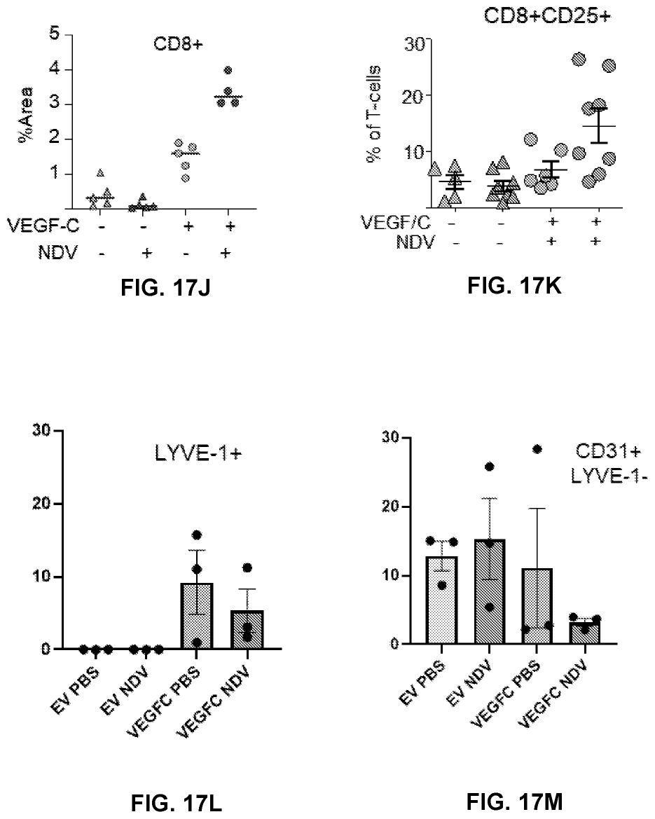

In agreement with the results obtained with flow cytometry, immunostaining of primary tumors demonstrated high density of CD8+ T-cells in NDV/VEGF-C tumors. CD4+ T-cells were also enriched, however, to a lesser extent than CD8+ T-cells. Analysis of myeloid cells in tumor showed particularly high densities of CD11c+ dendritic cells in NDV/VEGF-C tumors (FIGS. 17A-17D). Lymphatic vessels were prominent intratumorally and in particular peritumorally only in VEGF-C expressing tumors. Remarkably, a tight association between CD8+ T-cells and lymphatic vessels was observed (FIGS. 17G-171). Quantification of immunostained tumors showed a greater increase of CD8+ T-cells in tumor expressing VEGF-C and treated with NDV in comparison to tumors lacking VEGF-C (FIG. 17J). Conventional flow cytometry confirmed striking increase in effector memory T-cells in NDV/VEGF-C. Quantitative analysis of tumor vasculature showed increased lymphatic vessel densities in NDV/VEGF-C group and indicated that NDV alone was not driving lymphangiogenesis. All B16F10 tumors were highly vascularized and NDV/VEGF-C group in fact showed lower density of blood vessels compared to all other treatment groups. Taken together, these data demonstrate striking enrichment of CD8, CD4 and CD11c+ dendritic cells associated with tumor lymphatic vessels in NDV/VEGF-C-treated tumors.

6.5.2.4 Immunophenotyping of Sentinel and Contra-Lateral Lymph Nodes Reveals Changes in Immune Cell Subsets Unique for NDV/VEGF-C Tumors

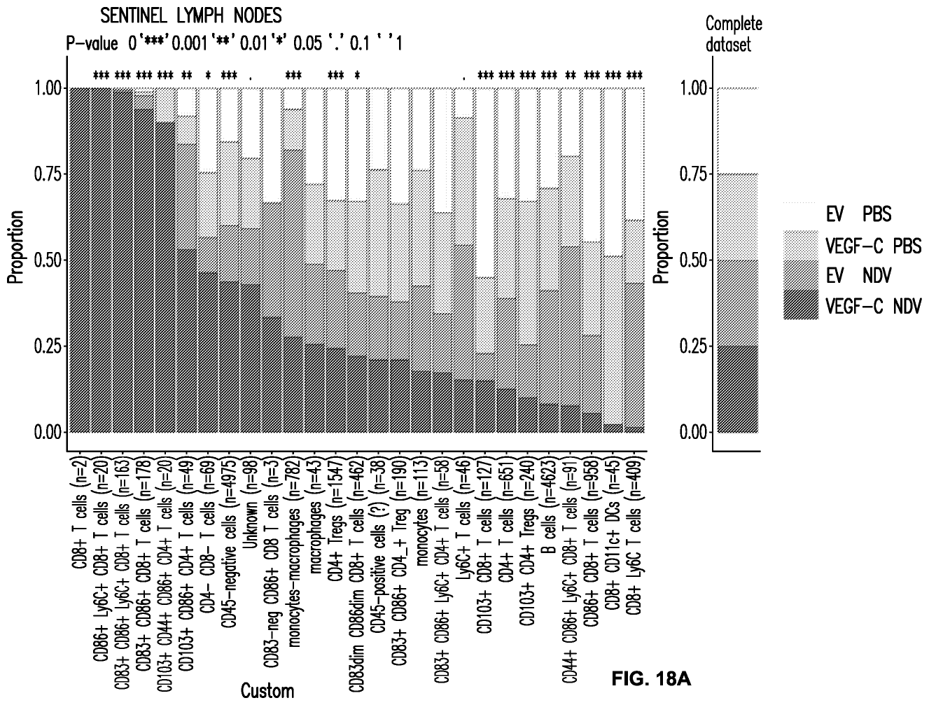

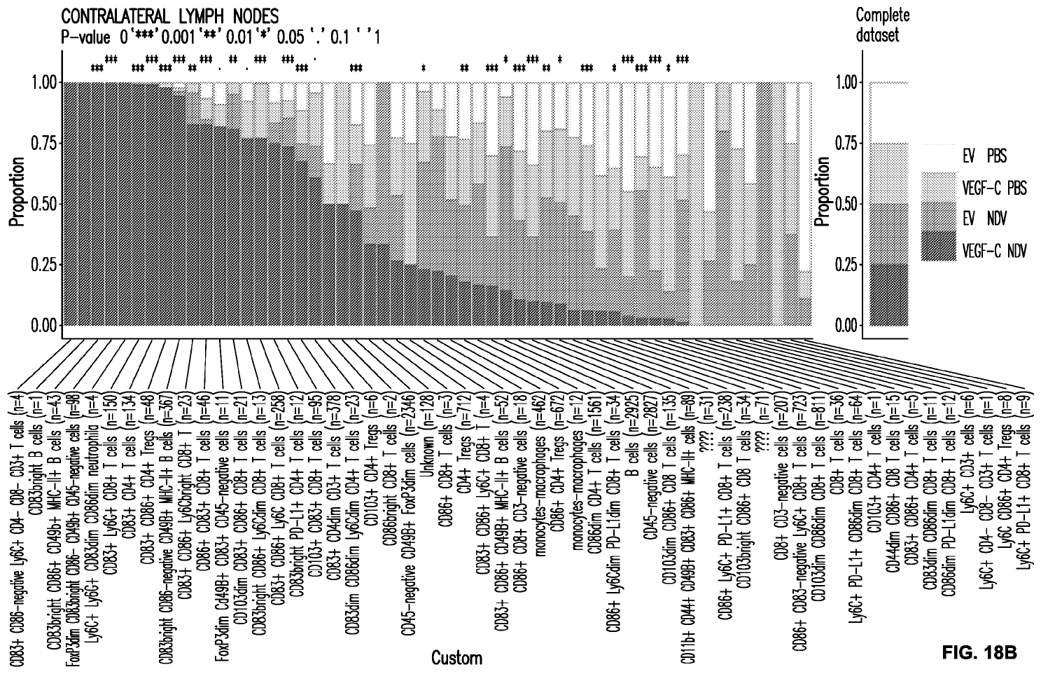

Numerous changes in immune cell phenotypes were observed in both, sentinel and contralateral lymph nodes in NDV/VEGF-C group compared to NDV alone and to non-treated groups (FIGS. 18A-18D). In particular, in sentinel lymph nodes an increase in CD4+ and CD8+ T cells expressing CD83 and/or CD86 has been noted, which typically indicates activated subsets. Interestingly, one subset unique to the NDV/VEGF-C group has CD103 (CD103+ CD44+ CD49b+ CD86+ CD4 T cells), and likely represents a tumor-specific subset (FIG. 18A). Contralateral lymph nodes do not drain tumor directly and changes of immune cells are a result of systemic changes, rather than a regional response to a tumor. Remarkably, several subsets highly enriched in contralateral lymph nodes in NDV/VEGF-C group, including CD83+ CD4 T cells, tumor-specific CD103+ CD83+ CD86+ CD8 T cells and CD83+ CD86+ Ly6c+ CD8 T cells were observed (FIG. 18B). Taken together, these data indicate that the NDV/VEGF-C combination leads to a potent immune activation of unique immune cell subsets both regionally (in sentinel lymph nodes) and systemically (in contralateral lymph nodes) that is strongly associated with tumor eradication.

### 6.6 Example 6: Construction of a Recombinant APMV4 Encoding a Codon Optimized hVEGF-C Gene

A new transcription unit is inserted at the restriction site Sal I that is created at the intergenic region between the viral genes P and M in the rescue plasmid pAPMV4 (see SEQ ID NO: 90 for plasmid pRz-APMV4 sequence without the additional transcription unit that is in bold). The DNA insert will be obtained by PCR, using as template a plasmid containing a codon optimized sequence encoding the human VEGF-C protein (SEQ ID NO: 35). The PCR primers are designed to introduce all the features required to generate a functional APMV4 transcription unit: The forward primer introduces the restriction site Sal I, the gene end sequence (transcription termination signal) from the viral gene HN, an intergenic sequence (1 nucleotide T), the gene start sequence (transcription initiation signal) from the viral gene HN and the Kozac sequence for efficient translation. The reverse primer introduces additional nucleotides as needed to comply with the rule of six, and a restriction site Sal I. The insert is cloned into the Sal I site of the rescue plasmid pAPMV4 by the technique In Fusion (GeneArt Seamless PLUS Cloning and Assembly Kit (ThermoFisher Scientific)).

Inserts containing a point mutation in the human VEGF-C sequence are generated as described above but two overlapping PCR products are generated: the first PCR product covers the sequence from the 5′ end of the transcription unit to the point mutation. The reverse primer contains the mutated sequence. The second PCR product covers the sequence from the point mutation to the 3′ end of the transcription unit and overlaps with the first PCR product by 15 nucleotides. The forward primer for the second PCR also contains the mutated sequence. Both PCR products are cloned into the Sal I site of the rescue plasmid pAPMV4 by the technique In Fusion. Two different point mutants are created: Cys156Ser and Cys137Ala.

Transcription units encoding the mature version of the human VEGF-C protein (delta N delta C), with or without point mutations, are created using the rescue plasmids containing the full length inserts described above (with and without point mutations, respectively) as templates. Two overlapping PCR products are created: the first PCR product contains the Sal I restriction site and APMV regulatory sequences described above and the signal sequence from an Immunoglobulin light chain (a potent signal peptide for protein secretion; SEQ ID NO: 25). The template is a plasmid that contains the sequence of the light chain signal peptide. The second PCR product overlaps in 15 nucleotides and contains the sequence encoding the mature form of the hVEGF-C (delta N delta C), followed by additional nucleotides as needed to comply with the rule of six, and a restriction site Sal I. The template for the second PCR is the rescue plasmid containing the full length, codon optimized sequence of the hVEGF-C protein, with or without the point mutations Cys156Ser or Cys137Ala.

All the constructs are confirmed by sequencing of the full length of the inserted sequences.

The invention is not to be limited in scope by the specific embodiments described herein. Indeed, various modifications of the invention in addition to those described will become apparent to those skilled in the art from the foregoing description and accompanying Figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

What is claimed:

1. A recombinant nucleic acid sequence comprising a nucleotide sequence of an avian paramyxovirus (APMV) genome and a transgene, wherein the transgene comprises a nucleotide sequence encoding vascular endothelial growth factor (VEGF)-C or VEGF-D.

2. The recombinant nucleic acid sequence of claim 1, wherein the nucleotide sequence of the genome comprises a transcription unit encoding a nucleocapsid (N) protein, a transcription unit encoding a phosphoprotein (P), a transcription unit encoding a matrix (M) protein, a transcription unit encoding a fusion (F) protein, a transcription unit encoding a hemagglutinin-neuraminidase (HN), and a transcription unit encoding a large polymerase (L) protein.

3. The recombinant nucleic acid sequence of claim 2, wherein the transgene is incorporated into the nucleotide sequence of the genome between the M and P transcription units or between the HN and L transcription units.

4. The recombinant nucleic acid sequence of any one of claims 1 to 3, wherein the APMV is Newcastle disease virus.

5. The recombinant nucleic acid sequence of claim 4, wherein the F protein of the Newcastle disease virus contains a leucine to alanine substitution at amino acid residue 289.

6. The recombinant nucleic acid sequence of claim 4 or 5, wherein the transgene comprises the nucleotide sequence of SEQ ID NO: 87.

7. The recombinant nucleic acid sequence of any one of claims 1 to 3, wherein the APMV is APMV serotype 4 (APMV-4).

8. The recombinant nucleic acid sequence of claim 7, wherein the transgene comprises the nucleotide sequence of SEQ ID NO: 89.

9. The recombinant nucleic acid sequence of any one of claim 1 to 5 or 7, wherein the nucleotide sequence encoding VEGF-C comprises the sequence set forth in any one of SEQ ID NOs: 1-18, 29-40, 49, or 50.

10. The recombinant nucleic acid sequence of any one of claim 1 to 5 or 7, wherein the VEGF-C comprises the amino acid sequence set forth in any one of SEQ ID NOs: 19-24, 41-46, 51, or 52.

11. The recombinant nucleic acid sequence of any one of claim 1 to 5 or 7, wherein the nucleotide sequence encoding VEGF-D comprises the sequence set forth in any one of SEQ ID NOs: 96-98.

12. The recombinant nucleic acid sequence of any one of claim 1 to 5 or 7, wherein the VEGF-D comprises the amino acid sequence set forth in any one of SEQ ID NOs: 99-104.

13. The recombinant nucleic acid sequence of claim 1 which comprises the nucleotide sequence of SEQ ID NO: 88 or 90.

14. A recombinant oncolytic virus comprising a genome that comprises a transgene, wherein the transgene comprises a nucleotide sequence encoding VEGF-C or VEGF-D.

15. The recombinant oncolytic virus of claim 14, wherein the virus is a parvovirus, a myxoma virus, a Newcastle disease virus, an APMV-2, an APMV-3, an APMV-4, an APMV-5, an APMV-6, an APMV-7, an APMV-8, an APMV-9, a reovirus, or Seneca valley virus.

16. The recombinant oncolytic virus of claim 14, wherein the virus is a genetically engineered influenza virus, measles virus, poliovirus, vaccinia virus, poxvirus, picornavirus, alphavirus, retrovirus, rhabdovirus, reovirus, adenovirus, herpes simplex virus, or vesicular stomatitis virus.

17. The recombinant oncolytic virus of any one of claims 14 to 16, wherein the nucleotide sequence encodes VEGF-C and the nucleotide sequence encoding VEGF-C comprises the sequence set forth in any one of SEQ ID NOs: 1-18, 29-40, 49, or 50.

18. The recombinant oncolytic virus of any one of claims 14 to 16, wherein the VEGF-C comprises the amino acid sequence set forth in any one of SEQ ID NOs: 19-24, 41-46, 51, or 52.

19. The recombinant oncolytic virus of any one of claims 14 to 16, wherein the nucleotide sequence encodes VEGF-D and the nucleotide sequence encoding VEGF-D comprises the sequence set forth in any one of SEQ ID NOs: 96-98.

20. The recombinant oncolytic virus of any one of claims 14 to 16, wherein the VEGF-D comprises the amino acid sequence set forth in any one of SEQ ID NOs: 99-104.

21. A recombinant avian paramyxovirus (APMV) comprising a packaged genome, wherein the packaged genome comprises a transgene that comprises a nucleotide sequence encoding VEGF-C or VEGF-D.

22. The recombinant APMV of claim 21, wherein the packaged genome comprises a transcription unit encoding a nucleocapsid (N) protein, a transcription unit encoding a phosphoprotein (P), a transcription unit encoding a matrix (M) protein, a transcription unit encoding a fusion (F) protein, a transcription unit encoding a hemagglutinin-neuraminidase (HN), and a transcription unit encoding a large polymerase (L) protein.

23. The recombinant APMV of claim 22, wherein the transgene is incorporated between the M and P transcription units or between the HN and L transcription units.

24. The recombinant APMV of any one of claims 21 to 23, wherein the APMV is Newcastle disease virus.

25. The recombinant APMV of claim 24, wherein the F protein of the Newcastle disease virus contains a leucine to alanine substitution at amino acid residue 289.

26. The recombinant APMV of claim 24 or 25, wherein the transgene comprises the nucleotide sequence of SEQ ID NO: 87.

27. The recombinant APMV of any one of claims 21 to 23, wherein the APMV is APMV-4.

28. The recombinant APMV of claim 27, wherein the transgene comprises the nucleotide sequence of SEQ ID NO: 89.

29. The recombinant APMV of any one of claim 21 to 25 or 27, wherein the nucleotide sequence encodes VEGF-C, and the nucleotide sequence encoding VEGF-C comprises the sequence set forth in any one of SEQ ID NOs: 1-18, 29-40, 49, or 50.

30. The recombinant APMV of any one of claim 21 to 25 or 27, wherein the VEGF-C comprises the amino acid sequence set forth in any one of SEQ ID NOs: 19-24, 41-46, 51, or 52.

31. The recombinant APMV of any one of claim 21 to 25 or 27, wherein the nucleotide sequence encodes VEGF-D, and the nucleotide sequence encoding VEGF-D comprises the sequence set forth in any one of SEQ ID NOs: 96-98.

32. The recombinant APMV of any one of claim 21 to 25 or 27, wherein the VEGF-D comprises the amino acid sequence set forth in any one of SEQ ID NOs: 99-104.

33. The recombinant APMV of claim 21, wherein the genome comprises a negative sense RNA transcribed from the cDNA sequence set forth in SEQ ID NO:88 or 90.

34. A pharmaceutical composition comprising the oncolytic virus of any one of claims 14 to 20 in a pharmaceutically acceptable carrier or excipient.

35. A pharmaceutical composition comprising the recombinant APMV of any one of claims 21 to 33 in a pharmaceutically acceptable carrier or excipient.

36. A method for treating cancer, comprising administering a dose of the pharmaceutical composition of claim 34 to a subject in need thereof.

37. A method for treating cancer, comprising administering a dose of the pharmaceutical composition of claim 35 to a subject in need thereof.

38. The method of claim 36 or 37, wherein the pharmaceutical composition is administered to the subject intratumorally.

39. The method of claim 36, 37 or 38, wherein the dose of the pharmaceutical composition contains 10⁶ to 10¹⁰ pfu of the virus.

40. The method of any one of claims 36 to 39, wherein the cancer is melanoma, lung carcinoma, colon carcinoma, glioblastoma, head and neck cancer, pancreatic cancer, hepatocellular carcinoma, ovarian cancer, squamous cell cancer, basal cell cancer, bladder cancer, prostate cancer, B-cell lymphoma, T-cell lymphoma, or breast cancer.

41. The method of any one of claims 36 to 40, wherein the cancer is metastatic.

42. The method of any one of claims 36 to 41, wherein the cancer is unresectable.

43. The method of any one of claims 36 to 42, wherein the subject is human.

44. A method for treating cancer, comprising administering intratumorally to a subject in need thereof a dose of a first pharmaceutical composition comprising an oncolytic virus and administering to the subject a dose of a second pharmaceutical composition comprising VEGF-C or VEGF-D.

45. A method for treating cancer, comprising administering intratumorally to a subject in need thereof a dose of a first pharmaceutical composition comprising an oncolytic virus and administering to the subject a dose of a second pharmaceutical composition comprising a nucleotide sequence encoding VEGF-C or VEGF-D.

46. The method of claim 45, wherein the nucleotide sequence encodes VEGF-C, and the nucleotide sequence that encodes VEGF-C comprises the sequence set forth in any one of SEQ ID NOs: 1-18, 29-40, 49, or 50.

47. The method of claim 44, wherein the VEGF-C comprises the amino acid sequence set forth in any one of SEQ ID NOs: 19-24, 41-46, 51, or 52.

48. The method of claim 45, wherein the nucleotide sequence encodes VEGF-D and the nucleotide sequence that encodes VEGF-D comprises the sequence set forth in any one of SEQ ID NOs: 96-98.

49. The method of claim 44, wherein the VEGF-D comprises the amino acid sequence set forth in any one of SEQ ID NOs: 99-104.

50. The method of any one of claim 44 or 46 to 49, wherein the second pharmaceutical composition is administered to the subject intratumorally, intramuscularly, intranasally, intradermally, or subcutaneously.

51. The method of claim 45, wherein the nucleotide sequence encoding VEGF-C comprises the sequence set forth in any one of SEQ ID NOs: 1-18, 29-40, 49, or 50.

52. The method of claim 45, wherein the VEGF-C comprises the amino acid sequence set forth in any one of SEQ ID NOs: 19-24, 41-46, 51, or 52.

53. The method of claim 45, wherein the nucleotide sequence encoding VEGF-D comprises the sequence set forth in any one of SEQ ID NOs: 96-98.

54. The method of claim 45, wherein the VEGF-D comprises the amino acid sequence set forth in any one of SEQ ID NOs: 99-104.

55. The method of any one of claim 45 or 51 to 54, wherein the second pharmaceutical composition is administered to the subject intratumorally, intramuscularly, intranasally, intradermally or subcutaneously.

56. The method of any one of claims 44 to 55, wherein the subject is not administered an antigen.

57. The method of any one of claims 44 to 56, wherein the dose of the first pharmaceutical composition contains 10⁶ to 10¹⁰ pfu of the virus.

58. The method of any one of claims 44 to 57, wherein the oncolytic virus is an APMV.

59. The method of claim 58, wherein the APMV is APMV-4.

60. The method of claim 58, wherein the APMV is Newcastle disease virus.

61. The method of any one of claims 44 to 60, wherein the cancer is melanoma, lung carcinoma, colon carcinoma, glioblastoma, head and neck cancer, pancreatic cancer, hepatocellular carcinoma, ovarian cancer, squamous cell cancer, basal cell cancer, bladder cancer, prostate cancer, B-cell lymphoma, T-cell lymphoma, or breast cancer.

62. The method of any one of claims 44 to 61, wherein the cancer is metastatic.

63. The method of any one of claims 44 to 62, wherein the cancer is unresectable.

64. The method of any one of claims 44 to 63, wherein the subject is human.


SEQUENCE LISTING (105 sequences)

SEQ ID NO: 1 - DNA, 1248 bp, Mus
  mouse VEGF-C full length wt native
atgcacttgctgtgcttcttgtctctggcgtgttccctgctcgccgctgcgctgatccccagtccgcgcgaggcgcccgccaccgtcgccgccttcgagtcgggactgggcttctcggaagcggagcccgacgggggcgaggtcaaggcttttgaaggcaaagacctggaggagcagttgcggtctgtgtccagcgtagatgagctgatgtctgtcctgtacccagactactggaaaatgtacaagtgccagctgcggaaaggcggctggcagcagcccaccctcaataccaggacaggggacagtgtaaaatttgctgctgcacattataacacagagatcctgaaaagtattgataatgagtggagaaagactcaatgcatgccacgtgaggtgtgtatagatgtggggaaggagtttggagcagccacaaacaccttctttaaacctccatgtgtgtccgtctacagatgtgggggttgctgcaacagcgaggggctgcagtgcatgaacaccagcacaggttacctcagcaagacgttgtttgaaattacagtgcctctctcacaaggccccaaaccagtcacaatcagttttgccaatcacacttcctgccggtgcatgtctaaactggatgtttacagacaagttcattcaattattagacgttctctgccagcaacattaccacagtgtcaggcagctaacaagacatgtccaacaaactatgtgtggaataactacatgtgccgatgcctggctcagcaggattttatcttttattcaaatgttgaagatgactcaaccaatggattccatgatgtctgtggacccaacaaggagctggatgaagacacctgtcagtgtgtctgcaagggggggcttcggccatctagttgtggaccccacaaagaactagatagagactcatgtcagtgtgtctgtaaaaacaaacttttccctaattcatgtggagccaacagggaatttgatgagaatacatgtcagtgtgtatgtaaaagaacgtgtccaagaaatcagcccctgaatcctgggaaatgtgcctgtgaatgtacagaaaacacacagaagtgcttccttaaagggaagaagttccaccatcaaacatgcagttgttacagaagaccgtgtgcgaatcgactgaagcattgtgatccaggactgtcctttagtgaagaagtatgccgctgtgtcccatcgtattggaaaaggccacatctgaactaa

SEQ ID NO: 2 - DNA, 1248 bp, Mus
  mouse VEGF-C full length Cys152Ser mutation native
atgcacttgctgtgcttcttgtctctggcgtgttccctgctcgccgctgcgctgatccccagtccgcgcgaggcgcccgccaccgtcgccgccttcgagtcgggactgggcttctcggaagcggagcccgacgggggcgaggtcaaggcttttgaaggcaaagacctggaggagcagttgcggtctgtgtccagcgtagatgagctgatgtctgtcctgtacccagactactggaaaatgtacaagtgccagctgcggaaaggcggctggcagcagcccaccctcaataccaggacaggggacagtgtaaaatttgctgctgcacattataacacagagatcctgaaaagtattgataatgagtggagaaagactcaatgcatgccacgtgaggtgtgtatagatgtggggaaggagtttggagcagccacaaacaccttctttaaacctccatctgtgtccgtctacagatgtgggggttgctgcaacagcgaggggctgcagtgcatgaacaccagcacaggttacctcagcaagacgttgtttgaaattacagtgcctctctcacaaggccccaaaccagtcacaatcagttttgccaatcacacttcctgccggtgcatgtctaaactggatgtttacagacaagttcattcaattattagacgttctctgccagcaacattaccacagtgtcaggcagctaacaagacatgtccaacaaactatgtgtggaataactacatgtgccgatgcctggctcagcaggattttatcttttattcaaatgttgaagatgactcaaccaatggattccatgatgtctgtggacccaacaaggagctggatgaagacacctgtcagtgtgtctgcaagggggggcttcggccatctagttgtggaccccacaaagaactagatagagactcatgtcagtgtgtctgtaaaaacaaacttttccctaattcatgtggagccaacagggaatttgatgagaatacatgtcagtgtgtatgtaaaagaacgtgtccaagaaatcagcccctgaatcctgggaaatgtgcctgtgaatgtacagaaaacacacagaagtgcttccttaaagggaagaagttccaccatcaaacatgcagttgttacagaagaccgtgtgcgaatcgactgaagcattgtgatccaggactgtcctttagtgaagaagtatgccgctgtgtcccatcgtattggaaaaggccacatctgaactaa

SEQ ID NO: 3 - DNA, 1248 bp, Mus
  mouse VEGF-C full length Cys133Ala mutation native
atgcacttgctgtgcttcttgtctctggcgtgttccctgctcgccgctgcgctgatccccagtccgcgcgaggcgcccgccaccgtcgccgccttcgagtcgggactgggcttctcggaagcggagcccgacgggggcgaggtcaaggcttttgaaggcaaagacctggaggagcagttgcggtctgtgtccagcgtagatgagctgatgtctgtcctgtacccagactactggaaaatgtacaagtgccagctgcggaaaggcggctggcagcagcccaccctcaataccaggacaggggacagtgtaaaatttgctgctgcacattataacacagagatcctgaaaagtattgataatgagtggagaaagactcaatgcatgccacgtgaggtggctatagatgtggggaaggagtttggagcagccacaaacaccttctttaaacctccatgtgtgtccgtctacagatgtgggggttgctgcaacagcgaggggctgcagtgcatgaacaccagcacaggttacctcagcaagacgttgtttgaaattacagtgcctctctcacaaggccccaaaccagtcacaatcagttttgccaatcacacttcctgccggtgcatgtctaaactggatgtttacagacaagttcattcaattattagacgttctctgccagcaacattaccacagtgtcaggcagctaacaagacatgtccaacaaactatgtgtggaataactacatgtgccgatgcctggctcagcaggattttatcttttattcaaatgttgaagatgactcaaccaatggattccatgatgtctgtggacccaacaaggagctggatgaagacacctgtcagtgtgtctgcaagggggggcttcggccatctagttgtggaccccacaaagaactagatagagactcatgtcagtgtgtctgtaaaaacaaacttttccctaattcatgtggagccaacagggaatttgatgagaatacatgtcagtgtgtatgtaaaagaacgtgtccaagaaatcagcccctgaatcctgggaaatgtgcctgtgaatgtacagaaaacacacagaagtgcttccttaaagggaagaagttccaccatcaaacatgcagttgttacagaagaccgtgtgcgaatcgactgaagcattgtgatccaggactgtcctttagtgaagaagtatgccgctgtgtcccatcgtattggaaaaggccacatctgaactaa

SEQ ID NO: 4 - DNA, 348 bp, Mus
  mouse VEGF-C mature (dNdC) wt native
gcacattataacacagagatcctgaaaagtattgataatgagtggagaaagactcaatgcatgccacgtgaggtgtgtatagatgtggggaaggagtttggagcagccacaaacaccttctttaaacctccatgtgtgtccgtctacagatgtgggggttgctgcaacagcgaggggctgcagtgcatgaacaccagcacaggttacctcagcaagacgttgtttgaaattacagtgcctctctcacaaggccccaaaccagtcacaatcagttttgccaatcacacttcctgccggtgcatgtctaaactggatgtttacagacaagttcattcaattattagacgt

SEQ ID NO: 5 - DNA, 348 bp, Mus
  mouse VEGF-C mature (dNdC) Cys152Ser mutation native
gcacattataacacagagatcctgaaaagtattgataatgagtggagaaagactcaatgcatgccacgtgaggtgtgtatagatgtggggaaggagtttggagcagccacaaacaccttctttaaacctccatctgtgtccgtctacagatgtgggggttgctgcaacagcgaggggctgcagtgcatgaacaccagcacaggttacctcagcaagacgttgtttgaaattacagtgcctctctcacaaggccccaaaccagtcacaatcagttttgccaatcacacttcctgccggtgcatgtctaaactggatgtttacagacaagttcattcaattattagacgt

SEQ ID NO: 6 - DNA, 348 bp, Mus
  mouse VEGF-C mature (dNdC) Cys133Ala mutation native
gcacattataacacagagatcctgaaaagtattgataatgagtggagaaagactcaatgcatgccacgtgaggtggctatagatgtggggaaggagtttggagcagccacaaacaccttctttaaacctccatgtgtgtccgtctacagatgtgggggttgctgcaacagcgaggggctgcagtgcatgaacaccagcacaggttacctcagcaagacgttgtttgaaattacagtgcctctctcacaaggccccaaaccagtcacaatcagttttgccaatcacacttcctgccggtgcatgtctaaactggatgtttacagacaagttcattcaattattagacgt

SEQ ID NO: 7 - DNA, 1251 bp, Mus
  mouse VEGF-C full length wt codon optimized 1 (MS)
atgcatctgctgtgtttcctgtcgctcgcctgctcactgctggcggcggcacttatcccgtccccacgggaggctcctgccaccgtggccgccttcgaatctgggctgggcttcagcgaagccgagcccgatggcggagaggtcaaggcattcgaaggaaaggacctcgaagaacagctgagatccgtgtcctccgtggacgaactcatgtccgtcctgtaccccgattactggaagatgtacaaatgccagctccggaagggcggttggcagcagcccactctgaacactcgcacgggagattccgtgaagtttgccgccgcccactacaatactgagattctcaagtccatcgacaacgaatggaggaaaacccagtgtatgccgcgcgaagtctgcattgacgtgggaaaggagttcggcgctgccaccaacaccttctttaagcctccctgcgtgtcggtgtatcgctgcgggggatgctgcaacagcgaaggccttcagtgcatgaacaccagcaccggatacctcagcaagactctcttcgaaatcactgtgccgctgtcacaaggcccgaagcctgtgaccatttccttcgccaaccacacctcctgtcggtgcatgagcaagctggatgtgtacagacaggtgcactccatcatccggagatcgttgcctgccaccctgccgcagtgccaagcggctaacaagacctgtcccaccaactacgtgtggaacaactatatgtgtcggtgcctggcacagcaggactttatcttctactccaacgtggaggacgactcgactaacggtttccacgacgtgtgcggacccaacaaggagctggatgaggatacttgtcagtgcgtgtgcaagggtggcctgcgcccgtcctcctgcggaccacataaggaactggacagggactcgtgccaatgcgtctgcaagaacaagctgttccctaactcctgcggggcgaaccgcgaattcgacgagaacacctgtcagtgtgtgtgcaagcggacttgcccgaggaatcagcctcttaaccccggaaaatgcgcctgcgaatgcacagagaacacccagaagtgcttcttgaaagggaagaagttccaccaccaaacctgttcatgctaccggcgcccatgtgccaaccggctgaagcactgcgacccgggattgagcttcagcgaggaggtctgcagatgcgtgccgtcatactggaagcgacctcatctgaattagtga

SEQ ID NO: 8 - DNA, 1251 bp, Mus
  mouse VEGF-C full length Cys152Ser mutation codon optimized 1 (MS)
atgcatctgctgtgtttcctgtcgctcgcctgctcactgctggcggcggcacttatcccgtccccacgggaggctcctgccaccgtggccgccttcgaatctgggctgggcttcagcgaagccgagcccgatggcggagaggtcaaggcattcgaaggaaaggacctcgaagaacagctgagatccgtgtcctccgtggacgaactcatgtccgtcctgtaccccgattactggaagatgtacaaatgccagctccggaagggcggttggcagcagcccactctgaacactcgcacgggagattccgtgaagtttgccgccgcccactacaatactgagattctcaagtccatcgacaacgaatggaggaaaacccagtgtatgccgcgcgaagtctgcattgacgtgggaaaggagttcggcgctgccaccaacaccttctttaagcctccctccgtgtcggtgtatcgctgcgggggatgctgcaacagcgaaggccttcagtgcatgaacaccagcaccggatacctcagcaagactctcttcgaaatcactgtgccgctgtcacaaggcccgaagcctgtgaccatttccttcgccaaccacacctcctgtcggtgcatgagcaagctggatgtgtacagacaggtgcactccatcatccggagatcgttgcctgccaccctgccgcagtgccaagcggctaacaagacctgtcccaccaactacgtgtggaacaactatatgtgtcggtgcctggcacagcaggactttatcttctactccaacgtggaggacgactcgactaacggtttccacgacgtgtgcggacccaacaaggagctggatgaggatacttgtcagtgcgtgtgcaagggtggcctgcgcccgtcctcctgcggaccacataaggaactggacagggactcgtgccaatgcgtctgcaagaacaagctgttccctaactcctgcggggcgaaccgcgaattcgacgagaacacctgtcagtgtgtgtgcaagcggacttgcccgaggaatcagcctcttaaccccggaaaatgcgcctgcgaatgcacagagaacacccagaagtgcttcttgaaagggaagaagttccaccaccaaacctgttcatgctaccggcgcccatgtgccaaccggctgaagcactgcgacccgggattgagcttcagcgaggaggtctgcagatgcgtgccgtcatactggaagcgacctcatctgaattagtga

SEQ ID NO: 9 - DNA, 1251 bp, Mus
  mouse VEGF-C full length Cys133Ala mutation codon optimized 1 (MS)
atgcatctgctgtgtttcctgtcgctcgcctgctcactgctggcggcggcacttatcccgtccccacgggaggctcctgccaccgtggccgccttcgaatctgggctgggcttcagcgaagccgagcccgatggcggagaggtcaaggcattcgaaggaaaggacctcgaagaacagctgagatccgtgtcctccgtggacgaactcatgtccgtcctgtaccccgattactggaagatgtacaaatgccagctccggaagggcggttggcagcagcccactctgaacactcgcacgggagattccgtgaagtttgccgccgcccactacaatactgagattctcaagtccatcgacaacgaatggaggaaaacccagtgtatgccgcgcgaagtcgccattgacgtgggaaaggagttcggcgctgccaccaacaccttctttaagcctccctgcgtgtcggtgtatcgctgcgggggatgctgcaacagcgaaggccttcagtgcatgaacaccagcaccggatacctcagcaagactctcttcgaaatcactgtgccgctgtcacaaggcccgaagcctgtgaccatttccttcgccaaccacacctcctgtcggtgcatgagcaagctggatgtgtacagacaggtgcactccatcatccggagatcgttgcctgccaccctgccgcagtgccaagcggctaacaagacctgtcccaccaactacgtgtggaacaactatatgtgtcggtgcctggcacagcaggactttatcttctactccaacgtggaggacgactcgactaacggtttccacgacgtgtgcggacccaacaaggagctggatgaggatacttgtcagtgcgtgtgcaagggtggcctgcgcccgtcctcctgcggaccacataaggaactggacagggactcgtgccaatgcgtctgcaagaacaagctgttccctaactcctgcggggcgaaccgcgaattcgacgagaacacctgtcagtgtgtgtgcaagcggacttgcccgaggaatcagcctcttaaccccggaaaatgcgcctgcgaatgcacagagaacacccagaagtgcttcttgaaagggaagaagttccaccaccaaacctgttcatgctaccggcgcccatgtgccaaccggctgaagcactgcgacccgggattgagcttcagcgaggaggtctgcagatgcgtgccgtcatactggaagcgacctcatctgaattagtga

SEQ ID NO: 10 - DNA, 348 bp, Mus
  mouse VEGF-C mature (dNdC) wt codon optimized 1 (MS)
gcccactacaatactgagattctcaagtccatcgacaacgaatggaggaaaacccagtgtatgccgcgcgaagtctgcattgacgtgggaaaggagttcggcgctgccaccaacaccttctttaagcctccctgcgtgtcggtgtatcgctgcgggggatgctgcaacagcgaaggccttcagtgcatgaacaccagcaccggatacctcagcaagactctcttcgaaatcactgtgccgctgtcacaaggcccgaagcctgtgaccatttccttcgccaaccacacctcctgtcggtgcatgagcaagctggatgtgtacagacaggtgcactccatcatccggaga

SEQ ID NO: 11 - DNA, 348 bp, Mus
  mouse VEGF-C mature (dNdC) Cys152Ser mutation codon optimized 1 (MS)
gcccactacaatactgagattctcaagtccatcgacaacgaatggaggaaaacccagtgtatgccgcgcgaagtctgcattgacgtgggaaaggagttcggcgctgccaccaacaccttctttaagcctccctccgtgtcggtgtatcgctgcgggggatgctgcaacagcgaaggccttcagtgcatgaacaccagcaccggatacctcagcaagactctcttcgaaatcactgtgccgctgtcacaaggcccgaagcctgtgaccatttccttcgccaaccacacctcctgtcggtgcatgagcaagctggatgtgtacagacaggtgcactccatcatccggaga

SEQ ID NO: 12 - DNA, 348 bp, Mus
  mouse VEGF-C mature (dNdC) Cys133Ala mutation codon optimized 1 (MS)
gcccactacaatactgagattctcaagtccatcgacaacgaatggaggaaaacccagtgtatgccgcgcgaagtcgccattgacgtgggaaaggagttcggcgctgccaccaacaccttctttaagcctccctgcgtgtcggtgtatcgctgcgggggatgctgcaacagcgaaggccttcagtgcatgaacaccagcaccggatacctcagcaagactctcttcgaaatcactgtgccgctgtcacaaggcccgaagcctgtgaccatttccttcgccaaccacacctcctgtcggtgcatgagcaagctggatgtgtacagacaggtgcactccatcatccggaga

SEQ ID NO: 13 - DNA, 1245 bp, Mus
  mouse VEGF-C full length wt codon optimized 2 (AGS)
atgcacctgctgtgcttcctgagcctggcctgcagcctgctggccgccgccctgatccccagccccagagaggcccccgccaccgtggccgccttcgagagcggcctgggcttcagcgaggccgagcccgacggcggcgaggtgaaggccttcgagggcaaggacctggaggagcagctgagaagcgtgagcagcgtggacgagctgatgagcgtgctgtaccccgactactggaagatgtacaagtgccagctgagaaagggcggctggcagcagcccaccctgaacaccagaaccggcgacagcgtgaagttcgccgccgcccactacaacaccgagatcctgaagagcatcgacaacgagtggagaaagacccagtgcatgcccagagaggtgtgcatcgacgtgggcaaggagttcggcgccgccaccaacaccttcttcaagcccccctgcgtgagcgtgtacagatgcggcggctgctgcaacagcgagggcctgcagtgcatgaacaccagcaccggctacctgagcaagaccctgttcgagatcaccgtgcccctgagccagggccccaagcccgtgaccatcagcttcgccaaccacaccagctgcagatgcatgagcaagctggacgtgtacagacaggtgcacagcatcatcagaagaagcctgcccgccaccctgccccagtgccaggccgccaacaagacctgccccaccaactacgtgtggaacaactacatgtgcagatgcctggcccagcaggacttcatcttctacagcaacgtggaggacgacagcaccaacggcttccacgacgtgtgcggccccaacaaggagctggacgaggacacctgccagtgcgtgtgcaagggcggcctgagacccagcagctgcggcccccacaaggagctggacagagacagctgccagtgcgtgtgcaagaacaagctgttccccaacagctgcggcgccaacagagagttcgacgagaacacctgccagtgcgtgtgcaagagaacctgccccagaaaccagcccctgaaccccggcaagtgcgcctgcgagtgcaccgagaacacccagaagtgcttcctgaagggcaagaagttccaccaccagacctgcagctgctacagaagaccctgcgccaacagactgaagcactgcgaccccggcctgagcttcagcgaggaggtgtgcagatgcgtgcccagctactggaagagaccccacctgaac

SEQ ID NO: 14 - DNA, 1245 bp, Mus
  mouse VEGF-C full length Cys152Ser mutation codon optimized 2 (AGS)
atgcacctgctgtgcttcctgagcctggcctgcagcctgctggccgccgccctgatccccagccccagagaggcccccgccaccgtggccgccttcgagagcggcctgggcttcagcgaggccgagcccgacggcggcgaggtgaaggccttcgagggcaaggacctggaggagcagctgagaagcgtgagcagcgtggacgagctgatgagcgtgctgtaccccgactactggaagatgtacaagtgccagctgagaaagggcggctggcagcagcccaccctgaacaccagaaccggcgacagcgtgaagttcgccgccgcccactacaacaccgagatcctgaagagcatcgacaacgagtggagaaagacccagtgcatgcccagagaggtgtgcatcgacgtgggcaaggagttcggcgccgccaccaacaccttcttcaagcccccctccgtgagcgtgtacagatgcggcggctgctgcaacagcgagggcctgcagtgcatgaacaccagcaccggctacctgagcaagaccctgttcgagatcaccgtgcccctgagccagggccccaagcccgtgaccatcagcttcgccaaccacaccagctgcagatgcatgagcaagctggacgtgtacagacaggtgcacagcatcatcagaagaagcctgcccgccaccctgccccagtgccaggccgccaacaagacctgccccaccaactacgtgtggaacaactacatgtgcagatgcctggcccagcaggacttcatcttctacagcaacgtggaggacgacagcaccaacggcttccacgacgtgtgcggccccaacaaggagctggacgaggacacctgccagtgcgtgtgcaagggcggcctgagacccagcagctgcggcccccacaaggagctggacagagacagctgccagtgcgtgtgcaagaacaagctgttccccaacagctgcggcgccaacagagagttcgacgagaacacctgccagtgcgtgtgcaagagaacctgccccagaaaccagcccctgaaccccggcaagtgcgcctgcgagtgcaccgagaacacccagaagtgcttcctgaagggcaagaagttccaccaccagacctgcagctgctacagaagaccctgcgccaacagactgaagcactgcgaccccggcctgagcttcagcgaggaggtgtgcagatgcgtgcccagctactggaagagaccccacctgaac

SEQ ID NO: 15 - DNA, 1245 bp, Mus
  mouse VEGF-C full length Cys133Ala mutation codon optimized 2 (AGS)
atgcacctgctgtgcttcctgagcctggcctgcagcctgctggccgccgccctgatccccagccccagagaggcccccgccaccgtggccgccttcgagagcggcctgggcttcagcgaggccgagcccgacggcggcgaggtgaaggccttcgagggcaaggacctggaggagcagctgagaagcgtgagcagcgtggacgagctgatgagcgtgctgtaccccgactactggaagatgtacaagtgccagctgagaaagggcggctggcagcagcccaccctgaacaccagaaccggcgacagcgtgaagttcgccgccgcccactacaacaccgagatcctgaagagcatcgacaacgagtggagaaagacccagtgcatgcccagagaggtggccatcgacgtgggcaaggagttcggcgccgccaccaacaccttcttcaagcccccctgcgtgagcgtgtacagatgcggcggctgctgcaacagcgagggcctgcagtgcatgaacaccagcaccggctacctgagcaagaccctgttcgagatcaccgtgcccctgagccagggccccaagcccgtgaccatcagcttcgccaaccacaccagctgcagatgcatgagcaagctggacgtgtacagacaggtgcacagcatcatcagaagaagcctgcccgccaccctgccccagtgccaggccgccaacaagacctgccccaccaactacgtgtggaacaactacatgtgcagatgcctggcccagcaggacttcatcttctacagcaacgtggaggacgacagcaccaacggcttccacgacgtgtgcggccccaacaaggagctggacgaggacacctgccagtgcgtgtgcaagggcggcctgagacccagcagctgcggcccccacaaggagctggacagagacagctgccagtgcgtgtgcaagaacaagctgttccccaacagctgcggcgccaacagagagttcgacgagaacacctgccagtgcgtgtgcaagagaacctgccccagaaaccagcccctgaaccccggcaagtgcgcctgcgagtgcaccgagaacacccagaagtgcttcctgaagggcaagaagttccaccaccagacctgcagctgctacagaagaccctgcgccaacagactgaagcactgcgaccccggcctgagcttcagcgaggaggtgtgcagatgcgtgcccagctactggaagagaccccacctgaac

SEQ ID NO: 16 - DNA, 348 bp, Mus
  mouse VEGF-C mature (dNdC) wt codon optimized 2 (AGS)
gcccactacaacaccgagatcctgaagagcatcgacaacgagtggagaaagacccagtgcatgcccagagaggtgtgcatcgacgtgggcaaggagttcggcgccgccaccaacaccttcttcaagcccccctgcgtgagcgtgtacagatgcggcggctgctgcaacagcgagggcctgcagtgcatgaacaccagcaccggctacctgagcaagaccctgttcgagatcaccgtgcccctgagccagggccccaagcccgtgaccatcagcttcgccaaccacaccagctgcagatgcatgagcaagctggacgtgtacagacaggtgcacagcatcatcagaaga

SEQ ID NO: 17 - DNA, 348 bp, Mus
  mouse VEGF-C mature (dNdC) Cys152Ser mutation codon optimized 2 (AGS)
gcccactacaacaccgagatcctgaagagcatcgacaacgagtggagaaagacccagtgcatgcccagagaggtgtgcatcgacgtgggcaaggagttcggcgccgccaccaacaccttcttcaagcccccctccgtgagcgtgtacagatgcggcggctgctgcaacagcgagggcctgcagtgcatgaacaccagcaccggctacctgagcaagaccctgttcgagatcaccgtgcccctgagccagggccccaagcccgtgaccatcagcttcgccaaccacaccagctgcagatgcatgagcaagctggacgtgtacagacaggtgcacagcatcatcagaaga

SEQ ID NO: 18 - DNA, 348 bp, Mus
  mouse VEGF-C mature (dNdC) Cys133Ala mutation codon optimized 2 (AGS)
gcccactacaacaccgagatcctgaagagcatcgacaacgagtggagaaagacccagtgcatgcccagagaggtggccatcgacgtgggcaaggagttcggcgccgccaccaacaccttcttcaagcccccctgcgtgagcgtgtacagatgcggcggctgctgcaacagcgagggcctgcagtgcatgaacaccagcaccggctacctgagcaagaccctgttcgagatcaccgtgcccctgagccagggccccaagcccgtgaccatcagcttcgccaaccacaccagctgcagatgcatgagcaagctggacgtgtacagacaggtgcacagcatcatcagaaga

SEQ ID NO: 19 - protein, 415 aa, Mus
  mouse VEGF-C full length wt
MHLLCFLSLACSLLAAALIPSPREAPATVAAFESGLGFSEAEPDGGEVKAFEGKDLEEQLRSVSSVDELMSVLYPDYWKMYKCQLRKGGWQQPTLNTRTGDSVKFAAAHYNTEILKSIDNEWRKTQCMPREVCIDVGKEFGAATNTFFKPPCVSVYRCGGCCNSEGLQCMNTSTGYLSKTLFEITVPLSQGPKPVTISFANHTSCRCMSKLDVYRQVHSIIRRSLPATLPQCQAANKTCPTNYVWNNYMCRCLAQQDFIFYSNVEDDSTNGFHDVCGPNKELDEDTCQCVCKGGLRPSSCGPHKELDRDSCQCVCKNKLFPNSCGANREFDENTCQCVCKRTCPRNQPLNPGKCACECTENTQKCFLKGKKFHHQTCSCYRRPCANRLKHCDPGLSFSEEVCRCVPSYWKRPHLN

SEQ ID NO: 20 - protein, 415 aa, Mus
  mouse VEGF-C full length Cys152Ser mutation
MHLLCFLSLACSLLAAALIPSPREAPATVAAFESGLGFSEAEPDGGEVKAFEGKDLEEQLRSVSSVDELMSVLYPDYWKMYKCQLRKGGWQQPTLNTRTGDSVKFAAAHYNTEILKSIDNEWRKTQCMPREVCIDVGKEFGAATNTFFKPPSVSVYRCGGCCNSEGLQCMNTSTGYLSKTLFEITVPLSQGPKPVTISFANHTSCRCMSKLDVYRQVHSIIRRSLPATLPQCQAANKTCPTNYVWNNYMCRCLAQQDFIFYSNVEDDSTNGFHDVCGPNKELDEDTCQCVCKGGLRPSSCGPHKELDRDSCQCVCKNKLFPNSCGANREFDENTCQCVCKRTCPRNQPLNPGKCACECTENTQKCFLKGKKFHHQTCSCYRRPCANRLKHCDPGLSFSEEVCRCVPSYWKRPHLN

SEQ ID NO: 21 - protein, 415 aa, Mus
  mouse VEGF-C full length Cys133Ala mutation
MHLLCFLSLACSLLAAALIPSPREAPATVAAFESGLGFSEAEPDGGEVKAFEGKDLEEQLRSVSSVDELMSVLYPDYWKMYKCQLRKGGWQQPTLNTRTGDSVKFAAAHYNTEILKSIDNEWRKTQCMPREVAIDVGKEFGAATNTFFKPPCVSVYRCGGCCNSEGLQCMNTSTGYLSKTLFEITVPLSQGPKPVTISFANHTSCRCMSKLDVYRQVHSIIRRSLPATLPQCQAANKTCPTNYVWNNYMCRCLAQQDFIFYSNVEDDSTNGFHDVCGPNKELDEDTCQCVCKGGLRPSSCGPHKELDRDSCQCVCKNKLFPNSCGANREFDENTCQCVCKRTCPRNQPLNPGKCACECTENTQKCFLKGKKFHHQTCSCYRRPCANRLKHCDPGLSFSEEVCRCVPSYWKRPHLN

SEQ ID NO: 22 - protein, 116 aa, Mus
  mouse VEGF-C mature (dNdC) wt
AHYNTEILKSIDNEWRKTQCMPREVCIDVGKEFGAATNTFFKPPCVSVYRCGGCCNSEGLQCMNTSTGYLSKTLFEITVPLSQGPKPVTISFANHTSCRCMSKLDVYRQVHSIIRR

SEQ ID NO: 23 - protein, 116 aa, Mus
  mouse VEGF-C mature (dNdC) Cys152Ser
AHYNTEILKSIDNEWRKTQCMPREVCIDVGKEFGAATNTFFKPPSVSVYRCGGCCNSEGLQCMNTSTGYLSKTLFEITVPLSQGPKPVTISFANHTSCRCMSKLDVYRQVHSIIRR

SEQ ID NO: 24 - protein, 116 aa, Mus
  mouse VEGF-C mature (dNdC) Cys133Ala
AHYNTEILKSIDNEWRKTQCMPREVAIDVGKEFGAATNTFFKPPCVSVYRCGGCCNSEGLQCMNTSTGYLSKTLFEITVPLSQGPKPVTISFANHTSCRCMSKLDVYRQVHSIIRR

SEQ ID NO: 25 - DNA, 60 bp, Artificial Sequence
  Signal peptide IgG light chain
atgcgggtgcccgcccagctgctgggcctgctcctgctctggctgccaggcgctagatgt

SEQ ID NO: 26 - protein, 20 aa, Artificial Sequence
  Signal peptide IgG light chain
MRVPAQLLGLLLLWLPGARC

SEQ ID NO: 27 - DNA, 51 bp, Artificial Sequence
  Signal peptide Gaussia luciferase
atgggcgtgaaggtgctgttcgccctgatctgcatcgccgtggccgaggcc

SEQ ID NO: 28 - protein, 17 aa, Artificial Sequence
  Signal peptide Gaussia luciferase
MGVKVLFALICIAVAEA

SEQ ID NO: 29 - DNA, 1260 bp, Homo sapiens
  human VEGF-C full length wt native
atgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaa

SEQ ID NO: 30 - DNA, 1260 bp, Homo sapiens
  human VEGF-C full length Cys156Ser mutation native
atgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatctgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaa

SEQ ID NO: 31 - DNA, 1260 bp, Homo sapiens
  human VEGF-C full length Cys137Ala mutation native
atgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtggctatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaa

SEQ ID NO: 32 - DNA, 348 bp, Homo sapiens
  human VEGF-C mature (dNdC) wt native
gcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgt

SEQ ID NO: 33 - DNA, 348 bp, Homo sapiens
  human VEGF-C mature (dNdC) Cys156Ser mutation native
gcacattataatacagagatcttgaaaagtattgataatgagtggagaaagactcaatgcatgccacgggaggtgtgtatagatgtggggaaggagtttggagtcgcgacaaacaccttctttaaacctccatctgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgt

SEQ ID NO: 34 - DNA, 348 bp, Homo sapiens
  human VEGF-C mature (dNdC) Cys137Ala mutation native
gcacattataatacagagatcttgaaaagtattgataatgagtggagaaagactcaatgcatgccacgggaggtggctatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgt

SEQ ID NO: 35 - DNA, 1260 bp, Homo sapiens
  human VEGF-C full length wt codon optimized 2 (AGS)
atgcacctgctgggcttcttcagcgtggcctgcagcctgctggccgccgccctgctgcccggccccagagaggcccccgccgccgccgccgccttcgagagcggcctggacctgagcgacgccgagcccgacgccggcgaggccaccgcctacgccagcaaggacctggaggagcagctgagaagcgtgagcagcgtggacgagctgatgaccgtgctgtaccccgagtactggaagatgtacaagtgccagctgagaaagggcggctggcagcacaacagagagcaggccaacctgaacagcagaaccgaggagaccatcaagttcgccgccgcccactacaacaccgagatcctgaagagcatcgacaacgagtggagaaagacccagtgcatgcccagagaggtgtgcatcgacgtgggcaaggagttcggcgtggccaccaacaccttcttcaagcccccctgcgtgagcgtgtacagatgcggcggctgctgcaacagcgagggcctgcagtgcatgaacaccagcaccagctacctgagcaagaccctgttcgagatcaccgtgcccctgagccagggccccaagcccgtgaccatcagcttcgccaaccacaccagctgcagatgcatgagcaagctggacgtgtacagacaggtgcacagcatcatcagaagaagcctgcccgccaccctgccccagtgccaggccgccaacaagacctgccccaccaactacatgtggaacaaccacatctgcagatgcctggcccaggaggacttcatgttcagcagcgacgccggcgacgacagcaccgacggcttccacgacatctgcggccccaacaaggagctggacgaggagacctgccagtgcgtgtgcagagccggcctgagacccgccagctgcggcccccacaaggagctggacagaaacagctgccagtgcgtgtgcaagaacaagctgttccccagccagtgcggcgccaacagagagttcgacgagaacacctgccagtgcgtgtgcaagagaacctgccccagaaaccagcccctgaaccccggcaagtgcgcctgcgagtgcaccgagagcccccagaagtgcctgctgaagggcaagaagttccaccaccagacctgcagctgctacagaagaccctgcaccaacagacagaaggcctgcgagcccggcttcagctacagcgaggaggtgtgcagatgcgtgcccagctactggaagagaccccagatgagctga

SEQ ID NO: 36 - DNA, 1260 bp, Homo sapiens
  human VEGF-C full length Cys156Ser mutation codon optimized 2 (AGS)
atgcacctgctgggcttcttcagcgtggcctgcagcctgctggccgccgccctgctgcccggccccagagaggcccccgccgccgccgccgccttcgagagcggcctggacctgagcgacgccgagcccgacgccggcgaggccaccgcctacgccagcaaggacctggaggagcagctgagaagcgtgagcagcgtggacgagctgatgaccgtgctgtaccccgagtactggaagatgtacaagtgccagctgagaaagggcggctggcagcacaacagagagcaggccaacctgaacagcagaaccgaggagaccatcaagttcgccgccgcccactacaacaccgagatcctgaagagcatcgacaacgagtggagaaagacccagtgcatgcccagagaggtgtgcatcgacgtgggcaaggagttcggcgtggccaccaacaccttcttcaagcccccctccgtgagcgtgtacagatgcggcggctgctgcaacagcgagggcctgcagtgcatgaacaccagcaccagctacctgagcaagaccctgttcgagatcaccgtgcccctgagccagggccccaagcccgtgaccatcagcttcgccaaccacaccagctgcagatgcatgagcaagctggacgtgtacagacaggtgcacagcatcatcagaagaagcctgcccgccaccctgccccagtgccaggccgccaacaagacctgccccaccaactacatgtggaacaaccacatctgcagatgcctggcccaggaggacttcatgttcagcagcgacgccggcgacgacagcaccgacggcttccacgacatctgcggccccaacaaggagctggacgaggagacctgccagtgcgtgtgcagagccggcctgagacccgccagctgcggcccccacaaggagctggacagaaacagctgccagtgcgtgtgcaagaacaagctgttccccagccagtgcggcgccaacagagagttcgacgagaacacctgccagtgcgtgtgcaagagaacctgccccagaaaccagcccctgaaccccggcaagtgcgcctgcgagtgcaccgagagcccccagaagtgcctgctgaagggcaagaagttccaccaccagacctgcagctgctacagaagaccctgcaccaacagacagaaggcctgcgagcccggcttcagctacagcgaggaggtgtgcagatgcgtgcccagctactggaagagaccccagatgagctga

SEQ ID NO: 37 - DNA, 1260 bp, Homo sapiens
  human VEGF-C full length Cys137Ala mutation codon optimized 2 (AGS)
atgcacctgctgggcttcttcagcgtggcctgcagcctgctggccgccgccctgctgcccggccccagagaggcccccgccgccgccgccgccttcgagagcggcctggacctgagcgacgccgagcccgacgccggcgaggccaccgcctacgccagcaaggacctggaggagcagctgagaagcgtgagcagcgtggacgagctgatgaccgtgctgtaccccgagtactggaagatgtacaagtgccagctgagaaagggcggctggcagcacaacagagagcaggccaacctgaacagcagaaccgaggagaccatcaagttcgccgccgcccactacaacaccgagatcctgaagagcatcgacaacgagtggagaaagacccagtgcatgcccagagaggtggccatcgacgtgggcaaggagttcggcgtggccaccaacaccttcttcaagcccccctgcgtgagcgtgtacagatgcggcggctgctgcaacagcgagggcctgcagtgcatgaacaccagcaccagctacctgagcaagaccctgttcgagatcaccgtgcccctgagccagggccccaagcccgtgaccatcagcttcgccaaccacaccagctgcagatgcatgagcaagctggacgtgtacagacaggtgcacagcatcatcagaagaagcctgcccgccaccctgccccagtgccaggccgccaacaagacctgccccaccaactacatgtggaacaaccacatctgcagatgcctggcccaggaggacttcatgttcagcagcgacgccggcgacgacagcaccgacggcttccacgacatctgcggccccaacaaggagctggacgaggagacctgccagtgcgtgtgcagagccggcctgagacccgccagctgcggcccccacaaggagctggacagaaacagctgccagtgcgtgtgcaagaacaagctgttccccagccagtgcggcgccaacagagagttcgacgagaacacctgccagtgcgtgtgcaagagaacctgccccagaaaccagcccctgaaccccggcaagtgcgcctgcgagtgcaccgagagcccccagaagtgcctgctgaagggcaagaagttccaccaccagacctgcagctgctacagaagaccctgcaccaacagacagaaggcctgcgagcccggcttcagctacagcgaggaggtgtgcagatgcgtgcccagctactggaagagaccccagatgagctga

SEQ ID NO: 38 - DNA, 348 bp, Homo sapiens
  human VEGF-C mature (dNdC) wt codon optimized 2 (AGS)
gcccactacaacaccgagatcctgaagagcatcgacaacgagtggagaaagacccagtgcatgcccagagaggtgtgcatcgacgtgggcaaggagttcggcgtggccaccaacaccttcttcaagcccccctgcgtgagcgtgtacagatgcggcggctgctgcaacagcgagggcctgcagtgcatgaacaccagcaccagctacctgagcaagaccctgttcgagatcaccgtgcccctgagccagggccccaagcccgtgaccatcagcttcgccaaccacaccagctgcagatgcatgagcaagctggacgtgtacagacaggtgcacagcatcatcagaaga

SEQ ID NO: 39 - DNA, 348 bp, Homo sapiens
  human VEGF-C mature (dNdC) Cys156Ser mutation codon optimized 2 (AGS)
gcccactacaacaccgagatcctgaagagcatcgacaacgagtggagaaagacccagtgcatgcccagagaggtgtgcatcgacgtgggcaaggagttcggcgtggccaccaacaccttcttcaagcccccctccgtgagcgtgtacagatgcggcggctgctgcaacagcgagggcctgcagtgcatgaacaccagcaccagctacctgagcaagaccctgttcgagatcaccgtgcccctgagccagggccccaagcccgtgaccatcagcttcgccaaccacaccagctgcagatgcatgagcaagctggacgtgtacagacaggtgcacagcatcatcagaaga

SEQ ID NO: 40 - DNA, 348 bp, Homo sapiens
  human VEGF-C mature (dNdC) Cys137Ala mutation codon optimized 2 (AGS)
gcccactacaacaccgagatcctgaagagcatcgacaacgagtggagaaagacccagtgcatgcccagagaggtggccatcgacgtgggcaaggagttcggcgtggccaccaacaccttcttcaagcccccctgcgtgagcgtgtacagatgcggcggctgctgcaacagcgagggcctgcagtgcatgaacaccagcaccagctacctgagcaagaccctgttcgagatcaccgtgcccctgagccagggccccaagcccgtgaccatcagcttcgccaaccacaccagctgcagatgcatgagcaagctggacgtgtacagacaggtgcacagcatcatcagaaga

SEQ ID NO: 41 - protein, 419 aa, Homo sapiens
  human VEGF-C full length wt
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 42 - protein, 419 aa, Homo sapiens
  human VEGF-C full length Cys156Ser mutation
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPSVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 43 - protein, 419 aa, Homo sapiens
  human VEGF-C full length Cys137Ala mutation
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVAIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 44 - protein, 116 aa, Homo sapiens
  human VEGF-C mature (dNdC) wt
AHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRR

SEQ ID NO: 45 - protein, 116 aa, Homo sapiens
  human VEGF-C mature (dNdC) Cys156Ser mutation
AHYNTEILKSIDNEWRKTQCMPREVCIDVGKEFGVATNTFFKPPSVSVYRCGGCCNSEGLQCMNTSTSYLSKTLFEITVPLSQGPKPVTISFANHTSCRCMSKLDVYRQVHSIIRR

SEQ ID NO: 46 - protein, 116 aa, Homo sapiens
  human VEGF-C mature (dNdC) Cys137Ala mutation
AHYNTEILKSIDNEWRKTQCMPREVAIDVGKEFGVATNTFFKPPCVSVYRCGGCCNSEGLQCMNTSTSYLSKTLFEITVPLSQGPKPVTISFANHTSCRCMSKLDVYRQVHSIIRR

SEQ ID NO: 47 - DNA, 35 bp, Artificial Sequence
  5′-non-coding-region-for-NDV
ccgcggttagaaaaaatacgggtagaaccgccacc

SEQ ID NO: 48 - DNA, 44 bp, Artificial Sequence
  5′-non-coding-region-for-APMV4
gtcgacatttttaattaaaatagggtggggaaggtaccgccacc

SEQ ID NO: 49 - DNA, 1668 bp, Canis lupus
  Nucleotide sequence for Canis lupus familiaris VEGF-C  GenBank No. XM_540047.6
cccgctccttccaccatgcacttgctgggcttctggtctctggcgtgttccctgctcgccgctgcgctgctgccggggccccgcgacgcgcccgccgccgccgccgccttcgaatcgggactcggcttctccgacgcggagcccgacgcgggcgaggcccaggcgtatgcaggcaaagatttggaggaacagttgcgatcagcgtccagtgtagatgaactcatgaccgtactctacccagaatattggaaaatgtacaagtgtcagttaaggaaaggcggctggcagcgtaataaagaacagcccaacatcagtgcaagaacagaagagactataaaatttgctgcagcacattataatgcagagatcttgaaaagtattgataatgagtggagaaaaactcagtgcataccacgtgaggtgtgtatagatgtggggaaggagtttggagcagcaacaaacaccttctttaaacctccatgtgtgtccgtctacagatgtggtggctgctgtaacagcgagggcctacagtgtatgaacaccagcacaagccacctcagcaagacgttgtttgaaattacagtgcctctctctcaaggccccaaaccagtaacaatcagttttgccaatcacacttcctgccgatgcatgtctaaactggacgtttacagacaagttcattccattattagacgttccctgccagcaacactaccacagtgccaagctgcaaacaagacttgccccacaaattacatctggaataatcatctctgcagatgcctggctcagcaagattttatttttgcctcaaattctggagatgactctacagatggattccatgacatctgtggacctaacaaggagctagatgaagaaacgtgtcagtgtgtctgcagaggggggctccggccttccagctgtggaccccacaaggaactagacagaaactcctgccagtgtgtctgtaaaaacaaactgttacccaactcgtgtggggccaacagagaatttgatgaaaacacgtgccagtgcgtatgtaaaagaacctgcccaagaaatcaacccctaaaccctggaaaatgtgcctgtgagtgtacagaaaattcacagaaatgcttcttaaaaggaaagaaatttcaacatcaaacatgcagctgttacagaagaccgtgtacaaaccgactgaggcattgtgagcaaggacttatatttagtgaagaagtatgtcgctgtgtcccttcatactggaaaagaccacagatgaactaagactgtactgttttccagtttgccatttctttatcttggaaaaccgtgttgccacattagaactatctgtgaacacagagaccttggtgggaccatggagacagagacagaagtcagtgtttgctgacctgtgtggataactttacagaaacggactggagctcatctgcaaaagacctcttttaatgactggtttttctgccaatgaccagacagctgaggtttttctcttgtgattaaaaaaaaaaaataatgactatataatttatttccactaaaaatattgtttctgcattcatgtttatagcaataacaattggtaaagctcactgtgatcaatatttttatatcatgcaaaatatgtttaaaataaaatgaaaattgtattataaa

SEQ ID NO: 50 - DNA, 1668 bp, Canis lupus
  Nucleotide sequence for Canis lupus dingo VEGF-C (GenBank Accession NO. XM_025434044)
cccgctccttccaccatgcacttgctgggcttctggtctctggcgtgttccctgctcgccgctgcgctgctgccggggccccgcgacgcgcccgccgccgccgccgccttcgaatcgggactcggcttctccgacgcggagcccgacgcgggcgaggcccaggcgtatgcaggcaaagatttggaggaacagttgcgatcagcgtccagtgtagatgaactcatgaccgtactctacccagaatattggaaaatgtacaagtgtcagttaaggaaaggcggctggcagcgtaataaagaacagcccaacatcagtgcaagaacagaagagactataaaatttgctgcagcacattataatgcagagatcttgaaaagtattgataatgagtggagaaaaactcagtgcataccacgtgaggtgtgtatagatgtggggaaggagtttggagcagcaacaaacaccttctttaaacctccatgtgtgtccgtctacagatgtggtggctgctgtaacagcgagggcctacagtgtatgaacaccagcacaagccacctcagcaagacgttgtttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgccaagctgcaaacaagacttgccccacaaattacatctggaataatcatctctgcagatgcctggctcagcaagattttatttttgcctcaaattctggagatgactctacagatggattccatgacatctgtggacctaacaaggagctagatgaagaaacgtgtcagtgtgtctgcagaggggggctccggccttccagctgtggaccccacaaggaactagacagaaactcctgccagtgtgtctgtaaaaacaaactgttacccaactcgtgtggggccaacagagaatttgatgaaaacacgtgccagtgcgtatgtaaaagaacctgcccaagaaatcaacccctaaaccctggaaaatgtgcctgtgagtgtacagaaaattcacagaaatgcttcttaaaaggaaagaaatttcaacatcaaacatgcagctgttacagaagaccgtgtacaaaccgactgaggcattgtgagcaaggacttatatttagtgaagaagtatgtcgctgtgtcccttcatactggaaaagaccacagatgaactaagactgtactgttttccagtttgccatttctttatcttggaaaaccgtgttgccacattagaactatctgtgaacacagagaccttggtgggaccatggagacagagacagaagtcagtgtttgctgacctgtgtggataactttacagaaacggactggagctcatctgcaaaagacctcttttaatgactggtttttctgccaatgaccagacagctgaggtttttctcttgtgattaaaaaaaaaaaataatgactatataatttatttccactaaaaatattgtttctgcattcatgtttatagcaataacaattggtaaagctcactgtgatcaatatttttatatcatgcaaaatatgtttaaaataaaatgaaaattgtattataaa

SEQ ID NO: 51 - protein, 419 aa, Canis lupus
  Canis Lupus Familiaris VEGF-C  (GenBank Accession No. XP_540047.2)
MHLLGFWSLACSLLAAALLPGPRDAPAAAAAFESGLGFSDAEPDAGEAQAYAGKDLEEQLRSASSVDELMTVLYPEYWKMYKCQLRKGGWQRNKEQPNISARTEETIKFAAAHYNAEILKSIDNEWRKTQCIPREVCIDVGKEFGAATNTFFKPPCVSVYRCGGCCNSEGLQCMNTSTSHLSKTLFEITVPLSQGPKPVTISFANHTSCRCMSKLDVYRQVHSIIRRSLPATLPQCQAANKTCPTNYIWNNHLCRCLAQQDFIFASNSGDDSTDGFHDICGPNKELDEETCQCVCRGGLRPSSCGPHKELDRNSCQCVCKNKLLPNSCGANREFDENTCQCVCKRTCPRNQPLNPGKCACECTENSQKCFLKGKKFQHQTCSCYRRPCTNRLRHCEQGLIFSEEVCRCVPSYWKRPQMN

SEQ ID NO: 52 - protein, 419 aa, Canis lupus; identical to SEQ ID NO: 51
  Canis Lupus Dingo VEGF-C (GenBank Accession No. XP_025289829.1)

SEQ ID NO: 53 - DNA, 36 bp, Artificial Sequence
  APMV4-PCR3-F
ccaagcttgcatgccgagcggccgtaaaattaacac

SEQ ID NO: 54 - DNA, 36 bp, Artificial Sequence
  APMV4-125-R
gattatctagattgtcagaacccataaagaatttgg

SEQ ID NO: 55 - DNA, 33 bp, Artificial Sequence
  APMV4-125-F
acaatctagataatcttgatatctaccagcagc

SEQ ID NO: 56 - DNA, 54 bp, Artificial Sequence
  APMV4-PCR3-R
cggtacccggggatcaagaaataaaagacatattttttattaaatattaatacg

SEQ ID NO: 57 - DNA, 37 bp, Artificial Sequence
  InF-N-APMV4-F
cacgataataccatggctggtgtcttctcccagtatg

SEQ ID NO: 58 - DNA, 48 bp, Artificial Sequence
  InF-N-APMV4-R
ttaggcctctcgagcctgcagctacagttcaaagtcgggttgatagtc

SEQ ID NO: 59 - DNA, 44 bp, Artificial Sequence
  InF-P-APMV4-F
cacgataataccatggattttactgacattgatgctgtcaactc

SEQ ID NO: 60 - DNA, 41 bp, Artificial Sequence
  InF-P-APMV4-R
ttaggcctctcgagcctgcagctagagcccaagggcttgtc

SEQ ID NO: 61 - DNA, 40 bp, Artificial Sequence
  InF-L-APMV4-F
cacgataataccatgtccaatcaggcagctgagattatac

SEQ ID NO: 62 - DNA, 45 bp, Artificial Sequence
  InF-L-APMV4-R
ttaggcctctcgagcctgcagctaaagtgagaggtagccccaacc

SEQ ID NO: 63 - DNA, 40 bp, Artificial Sequence
  APMV-mVEGFC-fwd
cacaccgtccgtcgacatttttaattaaaatagggtgggg

SEQ ID NO: 64 - DNA, 29 bp, Artificial Sequence
  APMV-mVEGFC-rev
ggcaagggatgtcgacccggtcagttcag

SEQ ID NO: 65 - DNA, 73 bp, Artificial Sequence
  mVEGFC-fwd
accgagttcccccccccgcggttagaaaaaatacgggtagaaccgccaccatgcatctgctgtgtttcctgtc

SEQ ID NO: 66 - DNA, 48 bp, Artificial Sequence
  mVEGFC-rev
ttggaccttgggtccgcgggctaattcagatgaggtcgcttccagtat

SEQ ID NO: 67 - DNA, 38 bp, Artificial Sequence
  APMV4-PCR1-1-F
ccaagcttgcatgccacgaaaaagaagaataaaaggca

SEQ ID NO: 68 - DNA, 19 bp, Artificial Sequence
  APMV4-PCR1-1-R
gggcgcgccactgagtctt

SEQ ID NO: 69 - DNA, 17 bp, Artificial Sequence
  APMV4-PCR1-2-F
ctcagtggcgcgcccca

SEQ ID NO: 70 - DNA, 19 bp, Artificial Sequence
  APMV4-PCR1-2-R
gatgtcgacggacggtgtg

SEQ ID NO: 71 - DNA, 18 bp, Artificial Sequence
  APMV4-PCR1-3-F
ccgtccgtcgacatccct

SEQ ID NO: 72 - DNA, 35 bp, Artificial Sequence
  APMV4-PCR1-3-R
cggtacccggggatccatcacctgcaggattacat

SEQ ID NO: 73 - DNA, 38 bp, Artificial Sequence
  APMV4-PCR2-1-F
ccaagcttgcatgcctaatcctgcaggtgatgaatctg

SEQ ID NO: 74 - DNA, 25 bp, Artificial Sequence
  APMV4-PCR2-1-R
gttcgatcgtttttaattaaaaagg

SEQ ID NO: 75 - DNA, 20 bp, Artificial Sequence
  APMV4-PCR2-2-F
taaaaacgatcgaactgagg

SEQ ID NO: 76 - DNA, 34 bp, Artificial Sequence
  APMV4-PCR2-2-R
cggtacccggggatcattttacggccgctcaggg

SEQ ID NO: 77 - DNA, 15054 bp, Artificial Sequence
  Avian paramyxovirus 4 strain APMV4/mallard/Belgium/15129/07, complete genome (GenBank JN571485.1)
gcgaaaaagaagaataaaaggcagaagccttttaaaaggaaccctgggctgtcgtaggtgtgggaaggttgtattccgagtgcgcctccgaggcatctactctacacctatcacaatggctggtgtcttctcccagtatgagaggtttgtggacaatcaatcccaagtatcaaggaaggatcatcggtccctggcagggggatgccttaaagtcaacatccctatgcttgtcactgcatctgaagatcccaccactcgttggcaactagcatgtttatctctaaggctcttgatctccaactcatcaaccagtgctatccgacagggggcaatactgactctcatgtcactaccgtcacaaaatatgagagcaacggcagctattgctggttccacaaatgcagctgttatcaacactatggaagtcttgagtgtcaatgactggaccccatccttcgaccctaggagcggtctctctgaagaggatgctcaggttttcagagacatggcaagggacctgccccctcagttcacctccggatcaccctttacatcagcattggcggaggggtttaccccagaagacacccacgacctaatggaggccttgaccagtgtgctgatacagatctggatcctggtggctaaggccatgaccaacattgatggctctggggaggccaatgagagacgtcttgcaaagtacatccaaaagggacagcttaatcgccagtttgcaattggtaatcctgctcgtctgataatccaacagacgatcaaaagctccttaactgtccgcaggttcttggtctctgagcttcgtgcatcacgaggtgcagtgaaagaaggatccccttactatgcagctgttggggatatccacgcttacatctttaacgcaggactgacaccattcttgactaccttaagatatgggataggcaccaagtatgctgctgttgcactcagtgtgttcgctgcagacattgcaaaattaaagagcctacttaccctgtaccaagacaagggtgtggaggccggatacatggcactccttgaagatccagattccatgcactttgcacccggaaatttcccacacatgtactcctatgcgatgggggtggcttcttaccatgaccccagcatgcgccaataccaatatgccaggaggttcctcagccgtcccttctacttgctagggagggacatggccgccaagaacacaggcacgctggatgagcaactggcaaaggaactgcaagtgtcagaaagagaccgcgccgcattgtccgctgcgattcaatcagcaatggaggggggagaatctgacgacttcccactgtcgggatccatgccggctctctccgacactgcgcaaccagttaccccaagaacccaacagtcccagctttcccctccacaatcatcaagcatgtctcaatcagcgcccaggaccccggactaccagcctgattttgaactgtaggctgcatccacgcaccaacaacaggcaaaagaaatcaccctcctccccacacatcccacccactcacccgccgagatccaatccaacaccccagcatccccatcatttaattaaaaactgaccaatagggtggggaaggagagttattggctgttgccaagtttgtgcagcaatggatttcaccgacattgatgctgtcaactcattaattgaatcatcatcagcaatcatagattccatacagcatggagggctgcaaccatcgggcactgtcggcctatcgcaaatcccaaaggggataaccagcgctttaactaaggcctgggaggctgaggcagcaactgctggcaatggggacacccaacacaaacctgacagtccggaggatcatcaggccaacgacacagactcccccgaagacacaggcaccaaccagaccatccaggaagccaatatcgttgaaacaccccaccccgaagtgctatcggcagccaaagccagactcaagaggcccaaggcagggagggacacccacgacaatccctctgcgcaacctgatcattttttaaaggggggccccctgagcccacaaccagcggcaccatgggtgcaaagtccacccattcatggaggtcccggcaccgtcgatccccgcccatcacaaactcaggatcattccctcaccggagagaaatggcaatcgtcaccgacaaagcaaccggagacattgaactggtggaatggtgcaacccggggtgcaccgcaatccgaactgaaccaaccagactcgactgtgtatgcggacactgccccaccatctgcagcctctgcatgtatgacgactgatcaggtacaactattaatgaaggaggttgccgatatgaaatcactccttcaggcattagtaaagaacctagctgtcctgcctcaactaaggaacgaggttgcagcaatcaggacatcacaggccatgatagaggggacactcaattcaatcaagattctcgatcctgggaattatcaagaatcatcactaaacagctggttcaaaccacgccaagatcacgcggttgttgtgtccggaccagggaatccattgaccatgccaaccccaatccaagacaacaccatattcctggatgaactggcaagacctcatcctagtttggtcaatccgtccccgcccactaccaacactaatgttgatcttggcccacagaagcaggctgcgatagcttatatctcagcaaaatgcaaggatccagggaaacgagatcagctctcaaagctcatcgagcgagcaaccaccttgagcgagatcaacaaagtcaaaagacaggccctcggcctctagatcactcgaccacccccagtaatgaatacaacaataatcagaacccccctaaaacacatggtcaacccaacacaccacccgcaccacccgctactatcctttgccagaaactccgccgcagccgatttattcaaaagaagccatttgatatgacttagcaaccgcaagatagggtggggaaggtgctttgcctgcaagagggctccctcatcttcagacacgtacccgccaacccaccagtgacgcaatggcagacatggacaccgtatatatcaatctgatggcagatgatccaacccaccaaaaagaactgctgtcctttcccctcgttcccgtgactggtcctgacgggaaaaaggaactccaacaccaggtccggactcaatccttgctcgcctcagacaagcaaactgagaggttcatcttcctcaacacttacgggtttatctatgacactacaccggacaagacaactttttccaccccagagcacatcaatcagcccaagagaacgatggtgagtgctgcgatgatgaccattggcctggtccccgccaatatacccttgaacgaattaacagctactgtgttcggcctgaaagtaagagtgaggaagagtgcgagatatcgagaggtggtctggtatcagtgcaatcctgtaccagccctgcttgcagccaccaggttcggtcgccaaggaggtctcgaatcaagcactggagtcagcgtaaaggcccccgagaagatagattgcgagaaggattatacttactacccttatttcctatctgtgtgctacatcgccacttccaacctgttcaaggtaccaaaaatggttgctaatgcgaccaacagtcaattataccacctgactatgcaggtcacatttgcctttccaaaaaacatccccccagctaaccagaaacttctgacacaagtggatgaaggattcgagggcactgtggactgccattttgggaacatgctgaaaaaggatcggaaagggaatatgaggacattgtcgcaggcggcagacaaggtcagacggatgaatatccttgttggtatctttgacttgcatgggccgacactcttcctggagtatactgggaaactaacaaaagctctgttagggttcatgtctactagccgaacagcaatcatccccatatctcagctcaatcctatgctgggtcaacttatgtggagcagtgatgcccagatagtaaaattaagagtggtcataactacatccaaacgcggcccatgcgggggtgagcaggagtatgtgctggatcccaaattcacagttaaaaaagagaaagcccgactcaaccctttcaagaaggcagcccaatgatcaaatctgcaggatctcaagaatcagaccactctatactattcaccgatcaatagacatgtaactatacagttgatggacctatgaagaatcaattagcaaaccgaatccttactagggtggggaaggagttgattgggtgtctaaacaaaagcattcctttacacctcctcgctacgaaacaaccataatgaggttatcacgcacaatcctgactttgattctcagcacacttaccggctatttaatgaatgcccactccaccaatgtgaatgagaaaccaaagtctgaggggattaggggggatcttataccaggcgcaggtatttttgtaactcaagtccgacaactacagatctaccaacagtctgggtatcatgaccttgtcatcaggttattacctcttctaccggcagaacttaatgattgtcaaagggaagttgtcacagagtacaacaacacggtatcacagctgttgcagcctatcaaaaccaacctggataccttattggctgatggtagcacaagggatgccgatatacagccacggttcattggggcaataatagccacaggtgccctggcggtggctacggtagctgaggtgactgcagcccaagcactatctcagtcgaaaacaaacgctcaaaatattctcaagttgagagatagtattcaggctaccaaccaagcagttttcgaaatttcacaaggactcgaggcaactgcaactgtgctatcaaaactgcaaactgagctcaatgagaacattatcccaagcctgaacaacttgtcctgtgctgccatggggaatcgccttggtgtatcactatcactctacttgaccttaatgaccactctatttggggaccagatcacaaacccagtgctgacaccaatctcctatagcactctatcggcaatggcaggcggtcacattggcccggtgatgagtaaaatattagctggatctgtcacaagtcagttgggggcagaacagttgattgctagcggcttaatacagtcacaggtagtaggttatgattcccaatatcaattattggttatcagggtcaaccttgtacggattcaagaggtccagaatacgagggtcgtatcactaagaacactagcggtcaatagggatggtggactttatagagcccaggtgcctcccgaggtagttgaacggtctggcattgcagagcgattttatgcagatgattgtgttcttactacaactgattacatttgctcatcgatccgatcttctcggcttaatccagagttagtcaagtgtctcagtggtgcacttgattcatgcacatttgagagggaaagtgcattattgtcgacccctttctttgtatacaacaaggcagtcgtcgcaaattgtaaagcagcaacatgtagatgtaataaaccgccatctattattgcccaatactctgcatcagctctagtcaccatcaccaccgacacctgtgccgaccttgaaattgagggttatcgcttcaacatacagactgaatccaactcatgggttgcaccaaacttcacggtctcgacttcacagattgtatcagttgatccaatagacatctcctctgacattgccaaaatcaacagttccatcgaggctgcgagagagcagctggaactgagcaaccagatcctttcccggatcaacccacgaattgtgaatgatgaatcactgatagctattatcgtgacaattgttgtgcttagtctccttgtaatcggtctgattgttgttctcggtgtgatgtataagaatcttaagaaagtccaacgagctcaagctgccatgatgatgcagcaaatgagctcatcacagcctgtgaccactaaattagggacgcctttctaggagaataatcatatcactctactcaatgatgagcaaaacgtaccaatcgtcaatgattgtgtcacgaggccggttgggaatgcatcgaatctctcccctttctttttaattaaaaacatttgaagtgagggtgagagggggggagtgtatggtagggtggggaaggtagccaattcctgcctattgggccgaccgtatcaaaagaactcaacagaagtctagatacagggtgacatggagggcagccgtgataatcttacagtggatgatgaattaaagacaacatggaggttagcttatagagttgtgtcccttctattgatggtgagcgctttgataatctctatagtaatcctgacaagagataacagccaaagcataatcacagcgatcaaccagtcatccgacgcagactcaaagtggcaaacgggaatagaagggaaaatcacctccattatgactgatacgctcgataccaggaatgcagcccttctccacattccactccagctcaacacgcttgaggcgaaccttttgtccgcccttgggggcaacacaggaattggtcccggggatctagatcactgccgttaccctgttcatgactccgcttacctgcatggagttaatcgattactcatcaaccagacagctgattacacagcagaaggccccctagatcatgtgaactttattccagccccggttacgaccactggatgcacaaggataccatccttttccgtgtcatcgtccatttggtgctatacacacaacgtgatcgaaaccggttgcaatgaccactcaggtagtaaccaatatatcagcatgggagtcattaagagagcgggcaacggcctaccttacttctcgacagttgtaagtaaatatctgactgatgggttgaataggaaaagctgttctgtagccgccggatccgggcattgctacctcctttgcagcttagtgtcggaacccgaacctgatgactatgtgtcacctgatcccacaccgatgaggttaggggtgctaacgtgggatgggtcttacactgaacaggtggtacccgaaagaatattcaagaacatatggagtgcaaactacccaggagtagggtcaggtgctatagtagggaataaggtgttattcccattttacggcggagtgagaaatggatcgaccccggaggtgatgaataggggaagatactactacatccaggatccaaatgactattgtcctgacccgctacaagatcagatcttaagggcggaacaatcgtattacccaactcgatttggtaggaggatggtaatgcaaggggtcctagcatgtccagtatccaacaattcaacaatagcaagccaatgtcaatcttactattttaataactcattaggattcattggggcagaatctagaatctattacctcaatggtaacatttacctttatcagagaagctcgagctggtggcctcatccccagatttacctgcttgattccaggattgcaagtccgggtactcagaacattgactcaggtgttaatctcaagatgttaaatgttactgtgattacacgaccatcatctggtttttgtaatagtcagtcacgatgccctaatgactgcttattcggggtctactcggatatctggcctcttagccttacctcagatagcatattcgcgttcacaatgtatttacaggggaagacaacacgtattgacccggcttgggcactattctccaatcatgcgattgggcatgaggctcgtctgttcaataagraggttagtgctgcttattctaccaccacttgtttttcggacactatccaaaatcaggtgtattgcctgagtatacttgaggtcaggagtgagctcttgggagcattcaaaatagtaccattcctctatcgcgtcttgtaggcatccattcagccaaaaaacttgagtgaccatgaggttaacacctgatccccttcaaaaacatctatcttaattaccgttctagatccatgattaggtacctttccaatcaatcatttggtttttaattaaaaacgaaagaatgggcctagttccaagaaagggctggaacccattagggtggggaaggattgctttgctccttgactcacacctgcgtacactcgatctcacttctataaagaaggaatccttctcaaattcgccccacaatgtccaatcaggcagctgagattatactacccaccttccatctagaatcacccttaatcgagaataagtgcttctattatatgcaattacttggtctcgtgttgccacatgatcactggagatggagggcattcgttaactttacagtggatcaggtgcaccttaaaaatcgtaatccccgcttaatggcccacatcgaccacactaaagatagattaaggactcatggtgtcttaggtttccaccagactcagacaagtatgagccgttaccgtgttttgcttcatcctgaaaccttaccttggctatcagccatgggaggatgcatcaatcaggttcctaaagcatggcggaacactctgaaatcgatcgagcacagtgtaaagcaggaggcacctcaactaaagttactcatggagagaacctcattaaaattaactggagtaccttacttgttctctaattgcaatcccgggaaaaccacagcaggaactatgcctgtcctaagtgagatggcatcggaactcttatcaaatcctatctcccaattccaatcaacatgggggtgtgctgcttcggggtggcaccatgtagtcagtatcatgaggctccaacaatatcaaagaaggacaggtaaggaagagaaagcaatcactgaagttcagtatggcacggacacctgtctcattaacgcagactacaccgttgttttttccacacagaaccgtgttataacggtcttgcctttcgatgttgtcctcatgatgcaagacctgctagaatcccgacggaatgtcctgttctgtgcccgctttatgtatcccagaagccaacttcatgagaggataagtacaatattagcccttggagaccaactggggagaaaagcaccccaagtcctgtatgattttgtagcaacccttgagtcatttgcatacgcagctgttcaacttcatgacaacaatcctacctacggtggggccttctttgaattcaatatccaagagttagaatctattctgtcccctgcacttagtaaggatcaggtcaacttctacataggtcaagtttgctcagcgtacagtaaccttcctccatctgaatcggcagaattgctgtgcctgctacgcctgtggggtcatcccttgctaaacagccttgatgcagcaaagaaagtcagggaatctatgtgtgccgggaaggttctcgattacaacgccattcgactcgtcttgtctttttatcatacgttactaatcaatgggtatcggaagaagcacaagggtcgctggccaaatgtgaatcaacattcactcctcaacccgatagtgaggcagctttattttgatcaggaggagatcccacactctgttgcccttgagcactatttggatgtctcaatgatagaatttgagaaaacttttgaagtggaactatctgacagcctaagcatcttcctgaaggataagtcgatagctttggacaagcaagaatggtacagtggttttgtctcagaagtgactccgaagcacctgcgaatgtcccgtcatgatcgcaagtctaccaataggctcctgttagccttcattaactcccctgaattcgatgttaaggaagagcttaaatacttgactacgggtgagtacgctactgacccaaatttcaatgtctcttactcactcaaagagaaggaagtaaagaaagaagggcgcattttcgcaaaaatgtcacaaaagatgagagcatgccaggttatttgtgaagaattgctagcacatcatgtggctcctttgtttaaagagaatggtgttactcaatcggagctatccctgacaaaaaatttgttggctattagccaactgagttacaactcgatggccgctaaggtgcgattgctgaggccaggggacaagttcactgctgcacactatatgaccacagacctaaagaagtactgtctcaattggcggcaccagtcagtcaaactgttcgccagaagcctggatcgactgtttgggctagaccatgctttttcttggatacatgtccgtctcaccaacagcactatgtacgttgctgaccccttcaatccaccagactcagatgcatgcacaaacttagacgacaataagaacaccgggatttttattataagtgcacgaggtggtatagaaggcctccaacaaaaactatggactggcatatcaatcgcaattgcccaagcagcagcagccctcgaaggcttacgaattgctgctactctgcagggggataaccaagttttggcgattacaaaggagttcatgaccccagtcccggaggatgtaatccatgagcagctatctgaggcgatgtcccgatacaaaaggactttcacatacctcaattatttaatggggcatcagttgaaggataaggaaaccatccaatccagtgatttctttgtgtactccaaaagaatcttcttcaatggatcaatcttaagtcaatgcctcaagaacttcagtaaactcactactaatgccactacccttgctgagaacactgtggccggctgcagtgacatctcttcatgcattgcccgttgtgtggaaaacgggttgcctaaggatgccgcatatattcagaatataatcatgactcggcttcaactattgctagatcattactattcaatgcatggcggcataaactcagaattagagcagccaactttaagtatctctgttcgaaacgcgacctacttaccatctcaactaggcggttacaatcatttgaatatgacccgactattctgccgcaatatcggcgacccgcttaccagttcttgggcggagtcaaaaagactaatggatgttggccttctcagtcgtaagttcttagaggggatattatggagacccccgggaagtgggacattttcaacactcatgcttgatccgttcgcacttaacattgattacctgaggccgccagagacaattatccgaaaacacacccaaaaagtcttgttgcaagattgcccaaatcccctattagcaggtgtcgttgacccgaactacaaccaagaattagagctattagctcagttcttgcttgatcgggaaaccgttatccccagggctgcccatgccatctttgaattgtctgtcttgggaaggaaaaaacatatacaaggattggtagatactacaaaaacaattattcagtgctcattggaaagacagccattgtcctggaggaaagttgagaacattgttacctacaacgcgcagtatttcctcggggccacccaacaggctgatactaatgtctcagaagggcagtgggtgatgccaggtaacttcaagaagcttgtgtcccttgacgattgctcagtcacgttgtccactgtatcgcggcgcatatcgtgggccaatctactgaactggagagctatagatggtttagaaaccccggatgtgatagagagtattgatggccgccttgtacaatcatccaatcaatgtggcctatgtaatcaagggttgggatcctactcctggttcttcttgccctctgggtgtgtgttcgaccgtccacaagattctcgggtagttccaaagatgccatacgtggggtccaaaacagatgagagacagactgcatcagtgcaagctatacagggatccacttgtcacctcagagcagcattgaggcttgtatcactctatctatgggcctatggagattctgacatatcatggctagaagctgcgacactggctcaaacacggtgcaatgtttctcttgatgacttgcgaatcttgagccctctcccttcttcggcgaatttacaccacagattaaatgacggggtaacacaggttaaattcatgcccgccacatcgagccgagtgtcaaagttcgtccaaatttgcaatgacaaccagaatcttatccgtgatgatgggagtgttgattccaatatgatttatcaacaagttatgatattggggcttggagagattgaatgcttgctagctgacccaatcgatacaaacccagaacaattgattcttcatctacactctgataattcttgctgtctccgggagatgccaacgaccggctttgtacctgctctaggactaaccccatgtttaactgtcccaaagcacaatccttacatttatgatgatagcccaatacccggtgatttggaccagaggctcatccagaccaaatttttcatgggttctgacaatttggataatcttgatatctaccaacagcgggctttattgagtaggtgtgtggcttatgatgttatccaatcgatatttgcttgtgatgcaccagtctctcagaagaatgacgcaatccttcacactgactatcatgagaattggatctcagagttccgatggggtgaccctcgtattatccaagtaacggcaggctacgagttaattctgttccttgcataccagctttattatctcagagtgaggggtgaccgtgcaatcctatgttatattgacaggatactcaacaggatggtatcttccaatctaggcagtctcatccagacactctctcatccagagattaggaggagattctcattgagtgatcaagggttccttgttgaaagggagctagagccaggtaagcccttggttaaacaagcggttatgttcttgagggactcggtccgctgcgctttagcaactatcaaggcaggaattgagcctgagatctcccgaggtggctgtactcaggatgagctgagctttactcttaagcacttactgtgtcggcgtctctgtgtaatcgctctcatgcattcagaagcaaagaacttggttaaagttagaaaccttcctgtagaagagaaaaccgccttactgtaccagatgttggtcactgaggccaatgctaggaaatcaggatctgctagcatcatcataaatctagtctcggcaccccagtgggacattcatacaccagcattgtattttgtatcaaagaaaatgctagggatgcttaaaaggtcaaccacacccttggatataagtgacctctccgagagccagaatcccgcacttgcagagctgaatgatgttcccggtcacatggcagaagaatttccctgtttgtttagtagttataacgccacatatgaagacacaattacttacaatccaatgactgaaaaactcgccttacacttggacaacagttccaccccatccagagcacttggtcgtcactacatcctgcggcctcttgggctctactcatccgcatggtaccggtctgcagcactactagcgtcaggggccctaaatgggttgcctgaggggtcgagcctgtacctaggagaagggtacgggaccaccatgactctgcttgagcccgttgtcaagtcttcaactgtttactaccatacattgtttgacccaacccggaatccttcacagcggaactataaaccagaaccacgggtattcacggattctatttggtacaaggatgatttcacacggccacctggtggtattatcaatctgtggggtgaagatatacgtcagagtgatatcacacagaaagacacggtcaacttcatactatctcagatcccgccaaaatcacttaagttgatacacgttgatattgagttctcaccagactccgatgtacggacactactatctggctattctcattgtgcactattggcctactggctattgcaacctggagggcgatttgcagttagagttttcttaagtgaccatatcatagtaaacttggtcactgcaatcctgtctgcttttgactctaatctggtgtgcattgcatcaggattgacacacaaggatgatggggcaggttatatttgcgcaaaaaagcttgcaaatgttgaggcttcaaggatcgagtactacttgaggatggtccatggttgtgttgactcattaaagatccctcatcaattaggaatcattaaatgggccgagggcgaggtgtcccaacttaccagaaaggcggatgatgaaataaattggcggttaggtgatccagttaccagatcatttgatccagtttctgagctaataattgcacgaacaggggggtctgtattaatggaatacggggcttttactaacctcaggtgtgcgaacttggcagatacatacaaacttctggcttcaattgtagagaccaccctaatggaaataagggttgagcaagatcaattagaagataattcgaggagacaaatccaagtagttcccgctttcaacactagatctgggggaaggatccgtacgctgattgagtgtgctcagctgcagattatagatgttatttgtgtaaacatagatcacctctttcctaaacaccgacatgttcttgtcacacaacttacctaccagtcagtgtgccttggggacttgattgaaggcccccaaattaagacgtatctaagggccaggaagtggatccaacgtcagggactcaatgagacagttaaccatatcatcactggacaagtgtcgcggaataaagcaagggattttttcaagaggcgtctgaagttggttggcttttcactctgcggtggttggagctacctctcactttagctgttcaggttgttgattattatgaataatcggagtcggaatcgtaaataggaagtcacaaagttgtgaataaacaatgattgcattagtatttaataaaaaatatgtcttttatttcgt

SEQ ID NO: 78 - DNA, 15054 bp, Artificial Sequence
  Avian paramyxovirus 4 APMV-4/duck/Hongkong/D3/75, complete genome (GenBank FJ177514.1)
acgaaaaagaagaataaaaggcagaagccttttaaaaggaaccctgggctgtcgtaggtgtgggaaggttgtattccgagtgcgcctccgaggcatctactctacacctatcacaatggctggtgtcttctcccagtatgagaggtttgtggacaatcaatcccaagtgtcaaggaaggatcatcggtccttagcaggaggatgccttaaagttaacatccctatgcttgtcactgcatctgaagaccccaccactcgttggcaactagcatgcttatctctaaggctcctgatctccaactcatcaaccagtgctatccgtcagggggcaatactgactctcatgtcattaccatcacaaaacatgagagcaacagcagctattgctggttccacaaatgcagctgttatcaacaccatggaagtcttaagtgtcaacgactggaccccatccttcgaccctaggagcggtctttctgaggaagatgctcaagttttcagagacatggcaagagatctgccccctcagttcacctctggatcacccttcacatcagcattggcggaggggttcactcctgaagatactcatgacctgatggaggccttgaccagtgtgctgatacagatctggatcctggtggctaaggccatgaccaacattgacggctctggggaggccaatgaaagacgtcttgcaaagtacatccaaaaaggacagcttaatcgtcagtttgcaattggtaatcctgcccgtctgataatccaacagacaatcaaaagctccttaactgtccgtaggttcttggtctctgagcttcgtgcgtcacgaggtgcagtaaaagaaggatccccttactatgcagctgttggggatatccacgcttacatctttaatgcgggattgacaccattcttgaccaccttaagatacgggataggcaccaagtacgccgctgttgcactcagtgtgttcgctgcagatattgcaaagttgaagagcctacttaccctgtaccaggacaagggtgtagaagctggatacatggcactccttgaggatccagactccatgcactttgcacctggaaacttcccacacatgtactcctatgcaatgggggtagcttcttaccatgatcctagcatgcgccaataccaatacgccaggaggttcctcagccgtcctttctacttactaggaagggacatggccgccaagaacacaggcacgctggatgagcaactggcgaaggaactgcaagtatcagagagagatcgcgccgcattatccgctgcgattcaatcagcgatggaggggggagagtccgacgacttcccactgtcgggatccatgccggctctctctgagaatgcgcaaccagttacccccagacctcaacagtcccagctctctcccccccaatcatcaaacatgccccaatcagcacccaggaccccagactatcaacccgactttgaactgtaggcttcatcaccgcaccaacaacagcccaagaagaccacccctccccccacacatctcacccagccacccataaagactcagtcccacgccccagcatctccttcatttaattaaaaaccgaccaacagggtggggaaggagagtcattggctactgccaattgtgtgcagcaatggattttactgacattgatgctgtcaactcattgatcgaatcatcatcggcaatcatagactccatacagcatggagggctgcaaccagcgggcaccgtcggcctatcgcagatcccaaaagggataaccagcgcattaaccaaggcctgggaggctgaggcggcaactgccggtaatggggacacccaacacaaatctgacagtccggaggatcatcaggccaacgacacagattcccctgaagacacaggtactgaccagaccacccaggaggccaacatcgttgagacaccccaccccgaggtgctgtcagcagccaaagccagactcaagaggcccaaagcagggagggacacccgcgacaactcccctgcgcaacccgatcatcttttaaaggggggcctcctgagcccacaaccagcagcatcatgggtgcaaaatccacccagtcatggaggtcccggcaccgccgatccccgcccatcacaaactcaggatcattcccccaccggagagaaatggcgattgtcaccgacaaagcaaccggagacattgaactggtggagtggtgcaacccggggtgcacagcagtccgaattgaacccaccagactcgactgtgtatgcggacactgccccaccatctgtagcctctgcatgtatgacgactgatcaggtacaactactaatgaaggaggttgctgacataaaatcactccttcaggcgttagtgaggaacctcgctgtcttgccccaattgaggaatgaggttgcagcaatcagaacatcacaggccatgatagaggggacactcaattcgatcaagattcttgaccctgggaattatcaggaatcatcactaaacagttggttcaaacctcgccaagatcacactgttgttgtgtctggaccagggaatccattggccatgccaaccccagtccaagacaacaccatattcctggacgagctagccagacctcatcctagtgtggtcaatccttccccacccatcaccaacaccaatgttgaccttggcccacagaagcaggctgcaatagcctatatctccgctaaatgcaaggatccggggaaacgagatcagctatcaaggctcattgagcgagcaaccaccccaagtgagatcaacaaagttaaaagacaagcccttgggctctagatcactcgatcacccctcatggtgatcacaacaataatcagaacccttccgaaccacatgaccaacccagcccaccgcccacaccgtccatcgacatcccttgccaaacatcctgccgtagctgatttattcaaaagagctcatttgatatgacctggtaatcataaaatagggtggggaaggtgctttgcctgtaagggggctccctcatcttcagacacgtgcccgccatctcaccaacagtgcaatggcagacatggacacggtgtatatcaatctgatggcagatgacccaacccaccaaaaagaactgctgtcctttcctctcatccctgtgaccggtcctgacgggaagaaggaactccaacaccagatccggacccaatccttgctcgcctcagacaaacaaactgaacggttcatcttcctcaacacttacggattcatctatgacaccacaccggacaagacaactttttccaccccagagcatattaatcagcctaagaggacgacggtgagtgccgcgatgatgaccattggcctggttcccgccaatatacccctgaacgaactaacggctactgtgttcagccttaaagtaagagtgaggaaaagtgcgaggtatcgggaagtggtctggtatcaatgcaatccagtaccggccctgcttgcagccaccaggtttggtcgccaaggaggtctcgagtcgagcactggagtcagtgtaaaggctcccgagaagatagattgtgagaaggattatacctactacccttatttcttatctgtgtgctacatcgccacctccaacctgttcaaggtaccgaggatggttgctaatgcaaccaacagtcaattataccaccttaccatgcaggtcacatttgcctttccaaaaaacatccctccagccaaccagaaactcctgacacaggtggatgagggattcgagggcactgtggattgccattttgggaacatgctgaaaaaggatcggaaagggaacatgaggacactgtcccaggcggcagataaggtcagacgaatgaatattcttgttggtatctttgacttgcatgggccaacgctcttcctggagtataccgggaaactgacaaaggctctgctagggttcatgtccaccagccgaacagcaatcatccccatatctcagctcaatcccatgctgagtcaactcatgtggagcagtgatgcccagatagtaaagttaagggttgtcataactacatccaaacgcggcccgtgcgggggtgagcaggagtatgtgctggatcccaaattcacagttaagaaagaaaaggctcgactcaaccctttcgagaaggcagcctaatgatttaatccgcaagatcccagaaatcagaccactctatactatccactgatcactggaaatgtaattgtacagttgatgaatctgtgaagaatcaattaaaaaaccggatccttattagggtggggaagtagttgattgggtgtctaaacaaaagcatttcttcacacctccccgccacgaaacaaccacaatgaggctatcaaacacaatcttgaccttgattctcatcatacttaccggctatttgataggtgtccactccaccgatgtgaatgagaaaccaaagtccgaagggattaggggtgatcttacaccaggtgcgggtattttcgtaactcaagtccgacagctccagatctaccaacagtctgggtaccatgatcttgtcatcagattgttacctcttctaccaacagagcttaatgattgtcaaagggaagttgtcacagagtacaataacactgtatcacagctgttgcagcctatcaaaaccaacctggatactttgttggcagatggtagcacaagggatgttgatatacagccgcgattcattggggcaataatagccacaggtgccctggctgtagcaacggtagctgaggtaactgcagctcaagcactatctcagtcaaaaacgaatgctcaaaatattctcaagttgagagatagtattcaggccaccaaccaagcagtttttgaaatttcacagggactcgaagcaactgcaaccgtgctatcaaaactgcaaactgagctcaatgagaatatcatcccaagtctgaacaacttgtcctgtgctgccatggggaatcgccttggtgtatcactctcactctatttgaccttaatgaccactctatttggggaccagatcacaaacccagtgctgacgccaatctcttacagcaccctatcggcaatggcgggtggtcacattggtccagtgatgagtaagatattagccggatctgtcacaagtcagttgggggcagaacaactgattgctagtggcttaatacagtcacaggtagtaggttatgattcccagtatcagctgttggttatcagggtcaaccttgtacggattcaggaagtccagaatactagggttgtatcactaagaacactagcagtcaatagggatggtggactttacagagcccaggtgccacccgaggtagttgagcgatctggcattgcagagcggttttatgcagatgattgtgttctaactacaactgattacatctgctcatcgatccgatcttctcggcttaatccagagttagtcaagtgtctcagtggggcacttgattcatgcacatttgagagggaaagtgcattactgtcaactcccttctttgtatacaacaaggcagtcgtcgcaaattgtaaagcagcgacatgtagatgtaataaaccgccatctatcattgcccaatactctgcatcagctctagtaaccatcaccaccgacacttgtgctgaccttgaaattgagggttatcgtttcaacatacagactgaatccaactcatgggttgcaccaaacttcacggtctcaacctcacaaatagtatcggttgatccaatagacatatcctctgacattgccaaaattaacaattctatcgaggctgcgcgagagcagctggaactgagcaaccagatcctttcccgaatcaacccacggattgtgaacgacgaatcactaatagctattatcgtgacaattgttgtgcttagtctccttgtaattggtcttattattgttctcggtgtgatgtacaagaatcttaagaaagtccaacgagctcaagctgctatgatgatgcagcaaatgagctcatcacagcctgtgaccaccaaattggggacacccttctaggtgaataatcatatcaatccattcaataatgagcgggacataccaatcaccaacgactgtgtcacaaggccggttaggaatgcaccggatctctctccttcctttttaattaaaaacggttgaactgagggtgagggggggggtgtgcatggtagggtggggaaggtagccaattcctgcccattgggccgaccgtaccaagagaagtcaacagaagtatagatgcagggcgacatggagggtagccgtgataacctcacagtagatgatgaattaaagacaacatggaggttagcttatagagttgtatccctcctattgatggtgagtgccttgataatctctatagtaatcctgacgagagataacagccaaagcataatcacggcgatcaaccagtcgtatgacgcagactcaaagtggcaaacagggatagaagggaaaatcacctcaatcatgactgatacgctcgataccaggaatgcagctcttctccacattccactccagctcaatacacttgaggcaaacctgttgtccgccctcggaggttacacgggaattggccccggagatctagagcactgtcgttatccggttcatgactccgcttacctgcatggagtcaatcgattactcatcaatcaaacagctgactacacagcagaaggccccctggatcatgtgaacttcattccggcaccagttacgactactggatgcacaaggatcccatccttttctgtatcatcatccatttggtgctatacacacaatgtgattgaaacaggttgcaatgaccactcaggtagtaatcaatatatcagtatgggggtgattaagagggctggcaacggcttaccttacttctcaacagtcgtgagtaagtatctgaccgatgggttgaatagaaaaagctgttccgtagctgcgggatccgggcattgttacctcctttgtagcctagtgtcagagcccgaacctgatgactatgtgtcaccagatcccacaccgatgaggttaggggtgctaacaagggatgggtcttacactgaacaggtggtacccgaaagaatatttaagaacatatggagcgcaaactaccctggggtagggtcaggtgctatagcaggaaataaggtgttattcccattttacggcggagtgaagaatggatcaacccctgaggtgatgaataggggaagatattactacatccaggatccaaatgactattgccctgacccgctgcaagatcagatcttaagggcagaacaatcgtattatcctactcgatttggtaggaggatggtaatgcagggagtcctaacatgtccagtatccaacaattcaacaatagccagccaatgccaatcttactatttcaacaactcattaggattcatcggggcggaatctaggatctattacctcaatggtaacatttacctttatcaaagaagctcgagctggtggcctcacccccaaatttacctacttgattccaggattgcaagtccgggtacgcagaacattgactcaggcgttaacctcaagatgttaaatgttactgtcattacacgaccatcatctggcttttgtaatagtcagtcaagatgccctaatgactgcttattcggggtttattcagatgtctggcctcttagccttacctcagacagcatatttgcatttacaatgtacttacaagggaagacgacacgtattgacccagcttgggcgctattctccaatcatgtaattgggcatgaggctcgtttgttcaacaaggaggttagtgctgcttattctaccaccacttgtttttcggacaccatccaaaaccaggtgtattgtctgagtatacttgaagtcagaagtgagctcttgggggcattcaagatagtgccattcctctatcgtgtcttataggcacctgcttggtcaagaaccctgagcagccataaaattaacacttgatcttccttaaaaacacctatctaaattactgtctgagatccctgattagttaccctttcaatcaatcaattaatttttaattaaaaacggaaaaatgggcctagttccaaggaaaggatgggacccattagggtggggaaggattactttgttccttgactcgcacccacgtacacccaatcccattcctgtcaagaaggaacccttcccaaactcaccttgcaatgtccaatcaggcagctgagattatactacccaccttccatcttttatcacccttgatcgagaataagtgcttctactacatgcaattacttggtctcgtgttaccacatgatcactggagatggagggcattcgtcaattttacagtggatcaagcacaccttaaaaatcgtaatccccgcttaatggcccacatcgatcacactaaggatagactaagggctcatggtgtcttgggtttccaccagactcagacaagtgagagccgtttccgtgtcttgctccatcctgaaactttaccttggctatcagcaatgggaggatgcatcaaccaggttcccaaggcatggcggaacactctgaaatctatcgagcacagtgtgaagcaggaggcgactcaactgaagttactcatggaaaaaacctcactaaagctaacaggagtatcttacttattctccaattgcaatcccgggaaaactgcagcgggaactatgcccgtactaagtgagatggcatcagaactcttgtcaaatcccatctcccaattccaatcaacatgggggtgtgctgcttcagggtggcaccatgtagtcagcatcatgaggctccaacagtatcaaagaaggacaggtaaggaagagaaagcaatcactgaagttcagtatggctcggacacctgtctcattaatgcagactacaccgtcgttttttccgcacaggaccgtgtcatagcagtcttgcctttcgatgttgtcctcatgatgcaagacctgcttgaatcccgacggaatgtcttgttctgtgcccgctttatgtatcccagaagccaactacatgagaggataagtacaatactggcccttggagaccaactcgggagaaaagcaccccaagtcctgtatgatttcgtagctaccctcgaatcatttgcatacgctgctgtccaacttcatgacaacaaccctatctacggtggggctttctttgagttcaatatccaagaactggaagctattttgtcccctgcacttaataaggatcaagtcaacttctacataagtcaagttgtctcagcatacagtaaccttcccccatctgaatcagcagaattgctatgcttactacgcctgtggggtcatcccttgctaaacagtcttgatgcagcaaagaaagtcagagaatctatgtgtgctgggaaggttcttgattataatgctattcgactagttttgtctttttatcatacgttattaatcaatgggtatcggaagaaacataagggtcgctggccaaatgtgaatcaacattcactactcaacccgatagtgaagcagctttactttgatcaggaggagatcccacactctgttgcccttgagcactatttagatatctcgatgatagaatttgagaagacttttgaagtggaactatctgatagtctaagcatctttctgaaggataagtcgatagctttggataaacaagaatggcacagtggttttgtctcagaagtgactccaaagcacctacgaatgtctcgtcatgatcgcaagtctaccaataggctattgttagcctttattaactcccctgaattcgatgttaaggaagagcttaaatatttgactacaggtgagtatgccactgacccaaatttcaatgtctcttactcactgaaagagaaggaagttaagaaagaagggcgcattttcgcaaagatgtcacagaaaatgagagcatgccaggttatttgtgaagagttactagcacatcatgtggctcctttgtttaaagagaatggtgttacacaatcggagctatccctgacaaagaatttgttggctattagccaactgagttacaactcgatggccgctaaggtgcgattgctgaggccaggggacaagttcaccgctgcacactatatgaccacagacctaaaaaagtactgccttaactggcggcaccagtcagtcaaattgttcgccagaagcctggatcgactatttgggttagaccatgctttttcttggatacacgtccgtctcaccaatagcactatgtacgttgctgacccattcaatccaccagactcagatgcatgcacaaatttagacgacaataagaacactgggatttttattataagtgctcgaggtggtatagaaggccttcaacagaaactatggactggcatatcaattgcaatcgcccaggcggcagcagccctcgagggcttacgaattgctgccactttgcagggggataaccaggttttagcgattacgaaagaattcatgaccccagtctcggaggatgtaatccacgagcagctatctgaagcgatgtcgcgatacaagaggactttcacataccttaattatttaatggggcaccaattgaaggataaagaaaccatccaatccagtgacttcttcgtttactccaaaaggatcttcttcaatgggtcaatcctaagtcaatgcctcaagaacttcagtaaactcactaccaatgccactacccttgctgagaacactgtagccggctgcagtgacatctcctcatgcatagcccgttgtgtggaaaacgggttgcctaaggatgctgcatatgttcagaatataatcatgactcggcttcaactgttgctagatcactactattctatgcatggtggcataaactcagagttagagcagccaactctaagtatccctgtccgaaacgcaacctatttaccatctcaattaggcggttacaatcatttgaatatgacccgactattctgtcgcaatatcggtgacccgcttactagttcttgggcagagtcaaaaagactaatggatgttggccttctcagtcgtaagttcttagaggggatattatggagacccccgggaagtgggacattttcaacactcatgcttgatccgttcgcacttaacattgattacttaaggccaccagagacaataatccgaaaacacacccaaaaagtcttgttgcaggattgtcctaatcctctattagcaggtgtagttgacccgaactacaaccaggaattagaattattagctcagttcctgcttgatcgggaaaccgttattcccagggctgcccatgccatctttgaactgtctgtcttgggaaggaaaaaacatatacaaggattggttgatactacaaaaacaattattcagtgctcattagaaagacagccactgtcctggaggaaagttgagaacattgtaacctacaatgcgcagtatttcctcggggccacccagcaggttgacaccaatatctcagaaaggcagtgggtgatgccaggtaatttcaagaagcttgtatctcttgacgattgctcagtcacgttgtccactgtgtcacggcgcatttcttgggccaatctacttaactggagggctatagatggtttggaaactccagatgtgatagagagtattgatggccgccttgtgcaatcatccaatcaatgcggcctatgtaatcaaggattgggctcctactcctggttcttcttgccctccgggtgtgtgttcgaccgtccacaagattctcgagtggttccaaagatgccatacgtgggatccaaaacggatgagagacagactgcgtcagtgcaggctatacagggatccacatgtcaccttagagcagcattgagacttgtatcactctacctttgggcctatggagattctgacatatcatggctagaagccgcgacattggctcaaacacggtgcaatatttctcttgatgacctgcggatcctgagccctcttccttcctcggcaaatttacaccacagattgaatgacggggtaacacaagtgaaattcatgcccgccacatcgagccgggtgtcaaagttcgtccaaatttgcaatgacaaccagaatcttatccgtgatgatgggagtgttgattccaatatgatttatcagcaggttatgatattagggcttggagagattgaatgtttgttagctgacccaatcgatacaaacccagaacaactgattcttcacctacactctgataattcttgctgtctccgggagatgccaacgaccggttttgtacctgctttaggattgaccccatgcttaactgtcccaaagcacaatccgtatatttatgatgatagcccaatacccggtgatttggatcagaggctcattcaaaccaaattctttatgggttctgacaatctagataatcttgatatctaccagcagcgagctttactgagtcggtgtgtggcttatgacattatccaatcagtattcgcttgcgatgcaccagtatctcagaagaatgatgcaatccttcacactgactaccatgaaaattggatctcagagttccgatggggtgaccctcgcataatccaagtaacagcaggttacgagttaattctgttccttgcataccagctttattatctcagagtgaggggtgaccgtgcaatcctgtgttatattgataggatactcaacaggatggtatcttccaatctaggcagtctcatccagacgctctctcatccggagattaggaggagattttcattgagtgatcaagggttccttgtcgaaagggagctagagccaggtaagccactggtaaaacaagcggttatgttcctaagggactcagtccgctgcgctttagcaactatcaaggcaggaattgagcctgagatctcccgaggtggctgtacccaggatgagctgagctttacccttaagcacttactatgtcggcgtctctgtataattgctctcatgcattcggaagcaaagaacttggtcaaagttagaaaccttccagtagaggaaaaaaccgccttactataccagatgttgatcactgaggccaatgccaggagatcagggtctgctagtatcatcataagcttagtttcagcaccccagtgggacattcatacaccagcgttgtattttgtatcaaagaaaatgctggggatgctcaaaaggtcaaccacacccttggatataagtgacctttctgagagccagaacctcacaccaacagaattgaatgatgttcctggtcacatggcagaggaatttccctgtttgtttagcagttataacgctacatatgaagacacaattacttacaatccaatgactgaaaaactcgcagtgcacttggacaatggttccaccccttccagagcgcttggtcgtcactacatcctgcgaccccttgggctttactcgtctgcatggtaccggtctgcagcactattagcgtcaggggccctcagtgggttgcctgaggggtcaagcctgtacttgggagaggggtatgggaccaccatgactctacttgagcccgttgtcaagtcctcaactgtttactaccatacattgtttgacccaacccggaatccttcacagcggaactacaaaccagaaccgcgggtattcactgattccatttggtacaaggatgatttcacacgaccacctggtggcattgtaaatctatggggtgaagacgtacgtcagagtgatattacacagaaagacacggttaatttcatattatctcgggtcccgccaaaatcactcaaattgatacacgttgatattgagttctccccagactctgatgtacggacgctactatctggctattcccattgtgcactattggcctactggctactgcaacctggagggcgatttgcggttagagttttcttaagtgaccatatcatagtcaacttggtcactgccattctgtccgcttttgactctaatctggtgtgcattgcgtcaggattgacacacaaggatgatggggcaggttatatttgtgcaaagaagcttgcaaatgttgaggcttcaagaattgagtattacttgaggatggtccacggctgtgttgactcattaaaaattcctcatcaattaggaatcattaaatgggctgagggtgaagtgtcccgacttaccaaaaaggcggatgatgaaataaactggcggttaggtgatccagttaccagatcatttgatccggtttctgagctaataattgcgcgaacagggggatcagtattaatggaatacgggacttttactaacctcaggtgtgcgaacttggcagatacatataaacttttggcttcaattgtagagaccaccttaatggaaataagggttgagcaagatcagttggaagatgattcgaggagacaaatccaggtagtccctgcttttaatacaagatccgggggaaggatccgtacattgattgagtgtgctcagctgcaggtcatagatgttatctgtgtgaacatagatcacctctttcccaaacaccgacatgctcttgtcacacaacttacttaccagtcagtgtgccttggggacttgattgaaggcccccaaattaagacatatctaagggccaggaagtggatccaacgtaggggactcaatgagacaattaaccatatcatcactggacaagtgtcgcggaataaggcaagggattttttcaagaggcgcctgaagttggttggcttttcgctctgtggcggttggggctacctctcactttagctgcttagattgttgattattatgaataatcggagtcgaaatcgtaaatagaaagacataaaattgcaaataagcaatgatcgtattaatatttaataaaaaatatgtcttttatttcgt

SEQ ID NO: 79 - DNA, 15054 bp, Artificial Sequence
  Avian paramyxovirus 4 isolate Uria_aalge/Russia/Tyuleniy_Island/115/2015, complete genome (GenBank KU601399.1)
acgaaaaagaagaataaaaggcagaagccttttaaaaggaaccctgggctgtcgtaggtgtgggaaggttgtattccgagtgcgcctccgaggcatctactctacacctatcacaatggctggtgtcttctcccagtatgagaggtttgtggataaccaatcccaagtgtcaaggaaggatcatcggtccctggcagggggatgcctcaaagtcaacatccctatgcttgtcactgcatctgaagatcccaccactcgttggcaactagcatgtttatctttaaggctcttgatctccaactcatcaaccagcgctatccgccagggggcaatactgactctcatgtcactaccatcacaaaatatgagagcaacggcagctattgctggttccacaaatgcagctgttatcaacactatggaagtcctaagtgtcaacgactggaccccatccttcgaccctaggagcggtctctctgaagaggatgctcaggtttttagagacatggcaagggatctgccccctcagttcacctccggatcaccctttacatcagctttggcggaggggtttaccccagaagacacccacgacctaatggaggccttgaccagtgtgctgatacagatctggatcctggtggctaaggccatgaccaacattgatggttctggggaggccaatgagagacgtcttgcaaagtatatccagaagggacagctcaatcgccagtttgcaattggtaatcctgctcgtctaataatccaacagacgatcaaaagctccttaactgtccgcaggttcttggtctctgagcttcgtgcatcacgaggtgcggtgaaagaaggatccccttattatgcagctgttggggatatccacgcatacatctttaacgcaggactgacaccattcttgactactttaagatatgggatcggcaccaagtatgctgctgttgcactcagtgtgttcgctgcagacattgcaaaattaaagagtctacttaccttataccaagataagggtgtggaggccggatacatggcactccttgaagatccagactccatgcactttgcacctggaaacttcccacacatgtactcctacgcgatgggggtggcttcttaccatgaccccagcatgcgccagtaccaatatgccaggaggttcctcagccgacccttctacttgctaggaagggacatggccgccaagaatacaggcacgctggatgagcaactggcaaaggaactgcaagtgtcagagagagaccgcgccgcactgtccgctgcgattcaatcagcaatggaagggggagaatccgacgacttcccactgtcgggatccatgccggctctctccgacaatgcacaaccagttaccccaagaacccaacagtcccagctctcccctccccaatcatcaagcatgtctcaatcagcgcccaggaccccggactaccagcctgattttgaactgtaggctgcatccatgcaccagcagcaggccaaagaaaccaccctcctctccacacatcccacccaatcacccgctgagactcaatccaacaccctagcatccccctcatttaattaaaaactgaccaatagggtggggaaggagagttattggctattgccaagttcgtgcagcaatggattttaccgatattgatgctgtcaactcattaatcgaatcatcatcagcaatcatagattccatacagcatggagggctgcaaccatcaggcactgtcggcctatcgcaaatcccaaaggggataaccagcgctttaaccaaagcctgggaggctgaggcagcaaatgctggcaatggggacacccaacaaaagtctgacagtctggaggatcatcaggccaacgacacagactcccccgaagacacaggcactaaccagaccatccaggaaaccaatatcgttgaaacaccccaccccgaagtgctatcggcagccaaagccagactcaagaggcccaaggcagggaaggacacccacgacaatccctctgcgcaacctgatcatcttttaaaggggggccccttgagcccacaaccagtggcaccgtgggtgcaaaatccgcccattcatggaggtcccggcaccgccgatccccgcccatcacaaactcaggatcattccctcaccggagagagatggcaatcgtcaccgacaaagcaaccggagccatcgaactggtggaatggtgcaacccggggtgcacagcaatccgaattgaacctaccagactcgactgtgtatgcggacactgccccaccatctgcagcctctgcatgtatgacgactgatcaggtacaactattaatgaaggaggttgccgatatgaaatcactccttcaggcactagtgaggaacctagctgtcctgcctcaactaaggaacgaggttgcagcaatcaggacatcacaggctatgatagaggggacacttaattcaatcaagattctcgaccctgggaattatcaggaatcatcactaaacagttggttcaaaccacgacaagatcacgcggttgttgtgtccggaccagggaatccattgaccatgccaaccccaatccaggacaataccatattcctggatgaattggcaagacctcatcctagtttggtcaatccgtccccgcccactaccaacactaatgttgatcttggcccacagaagcaggctgcgatagcttatatctcagcaaaatgcaaggatcaagggaaacgagatcagctctcaaagctcatcgagcgagcaaccaccttgagtgagatcaacaaagttaaaagacaggctcttggcctctagatcacccaatcacccccagtaatgagtacaacaataatcagaacctccctaaaccacatggccaaccaagcacaccatccacaccaccccttactatcctttgccagaaactccgccgcagctgatttattcaaaagaagccacttggtataacctagcaaccgcaagatagggtggggaaggtgctttgcctgcaagagggctccctcatcttcagacacttacccgccaacccaccagtgacacaatggcagacatggacactgtatatatcaatctgatggcagatgatccaacccaccaaaaagaactgctgtcctttcccctcattccagtgactggtcccgacgggaaaaaggaactccaacaccaggttcggactcaatccttgctcgcctcagacaagcaaactgagaggttcatcttcctcaacacttacgggtttatctatgacactacaccggacaagacaactttttccaccccagagcatatcaatcagcccaagagaacgatggtgagtgctgcaatgatgaccatcggcctggtccccgccaatatacccttgaacgaactaacagctactgtgtttggcctgaaggtgagagtgaggaagagtgcgagatatcgagaggtggtctggtatcagtgcaaccctgtaccagccctgctggcagccaccaggttcggtcgccaagggggtctcgaatcgagcactggagtcagtgtgaaggcccctgagaagatagattgtgagaaggattatacttactacccttatttcctatctgtgtgctacatcgctacttccaacctgttcaaggtaccaaaaatggttgctaatgcgaccaacagtcaattataccatctgaccatgcaggtcacatttgcctttccaaaaaacatccccccagctaaccagaaactcctgacacaagtggatgaaggattcgagggcactgtggactgccattttgggaacatgctgaaaaaggatcggaaagggaatatgaggacattgtcgcaggcggcagataaggtcagacggatgaacatccttgttggtatctttgacttgcatgggccgacactcttcctggagtataccgggaaactaacaaaagctctgctagggttcatgtctaccagccgaacagcaatcatccccatatctcagctcaatcctatgctgagtcaactcatgtggagtagtgatgcccagatagtaaaattaagagtggtcataactacatccaaacgcggcccatgcgggggtgagcaggagtatgtgctggatcccaaattcacagttaaaaaagaaaaagcccgactcaatcctttcaagaaggcagcccaatgatcaaatctgcaggatctcagaaatcagaccactctatactatccactgattaatagacacgtagctatacagttgatgaacctatgaagaatcaattagcaaaccgaatccttgctagggtggggaaggagttgattgggtgtctaaacaaaagcactcctttgcacctcctcgccacgaaacaaccataatgaggttatcacgcacaatcctggccctgattctaggcacacttaccggctatttaatggatgcccactccaccactgtgaacgagagaccaaagtctgaagggattaggggtgatcttataccaggcgcaggtatctttgtaactcaagtccgacaactacagatctaccaacagtctgggtatcatgaccttgtcatcaggttattacctcttctaccggcagaactcaatgattgtcaaagggaagttgtcacagagtacaacaatacggtatcacagctgttgcagcctatcaaaaccaacctggataccttattggctgatggtggtacaagggatgccgatatacagccgcggttcattggggcgataatagccacaggtgccctggcggtggctacggtagctgaggtgactgcagcccaagcactatcgcagtcgaaaacgaacgctcaaaatattctcaagttgagagatagtattcaggccaccaaccaggcagtttttgaaatttcacaaggacttgaggcaactgcaactgtgctatcaaaactgcaaactgagctcaatgagaacattatcccaagcctgaacaacttgtcctgtgctgctatggggaatcgccttggtgtatcactatcactctacttgaccttaatgaccaccctatttggggaccagatcacaaacccagtgctgacaccaatctcctatagcactctatcggcaatggcaggtggtcacattggcccggtgatgagtaagatattagccggatctgtcacaagtcagttgggggcagaacagttgattgctagcggcttaatacagtcacaagtagtgggttatgattcccaatatcaattattggttatcagggtcaatcttgtacggattcaagaggtccagaatacgagggtcgtatcactaagaacactagcggtcaatagggatggtggactttatagagcccaggtgcctcctgaggtagttgaacggtctggcattgcagagcgattttacgcagatgattgcgttcttactacaactgattacatttgctcatcgatccgatcttctcggcttaatccagagttagtcaagtgtctcagtggggcacttgattcatgcacatttgagagggaaagtgcattattgtcaacccctttctttgtatacaacaaggcagttgtcgcaaattgtaaagcagcaacatgtagatgtaataaaccgccgtctattattgcccaatactctgcatcggctctggtcaccatcaccactgacacctgcgccgaccttgaaattgagggttatcgcttcaacatacagactgaatccaactcatgggttgcaccaaacttcactgtctcgacttcacagattgtatcagttgatccaatagacatctcctctgacattgccaaaatcaacagttccatcgaggctgcaagagagcagctggaactaagcaaccagatcctctcccggattaacccacgaatcgtgaatgatgaatcactgatagctattatcgtgacaattgttgtgcttagtctcctcgtaatcggtctgattgttgttctcggtgtgatgtataagaatcttaagaaagtccaacgagctcaagctgccatgatgatgaagcaaatgagctcatcacagcctgtgaccactaaattagggacgcctttctaggaggataatcatattactctactcaatgatgagcaagacgtaccaattatcaatgattgtgtcacaaggccggttgggaatgcaccgaatctctcccctttctttttaattaaaaacatttgaagtgaggataagaggggggaagagtatggtagggtggggaaggtagccaatccctgcctattaggctgatcgtatcaaaagaacccaacagaagtctagatacagggcaacatggagggcagccgtgataatctaacagtggatgatgaattaaagacaacatggaggttagcttatagagttgtgtccctcctattgatggtgagcgctttgataatctctatagtaatcctgacaagagataacagccaaagcataatcacggcgatcaaccagtcatctgacgcagactctaagtggcaaacgggaatagaagggaaaatcacctccattatgactgatacgctcgataccagaaatgcagcccttctccacattccactccagctcaacacgcttgcggcgaacctattgtccgcccttggaggcaacacaggaattggccccggagatctggaacactgccgttaccctgttcatgacaccgcttacctgcatggagttaatcgattactcatcaaccagacagctgattatacagcagaaggccccctagatcatgtgaacttcataccagccccggttacgaccactggatgcacaaggataccatccttttctgtgtcatcgtccatttggtgctatacacacaacgtgattgaaaccggttgcaatgaccactcaggtagtaaccaatatatcagcatgggagtcattaagagagcaggcaacggcttaccttacttctcaacagttgtaagtaagtatctgactgatgggttgaataggaagagctgttctgtagctgccggatctgggcattgctacctcctttgcagcttagtgtcggagcctgaacctgatgactatgtatcacctgatcccacaccgatgaggttaggggtgctaacgtgggatgggtcttacactgaacaggtggtacccgaaagaatattcaagaacatatggagtgcaaactacccgggagtagggtcaggtgctatagtaggaaataaagtgttattcccattttacggcggagtgaggaatggatcgaccccggaggtgatgaataggggaagatactactacatccaggatccaaatgactattgccctgacccgctgcaagatcagatcttaagagcggaacaatcgtattacccaactcgattcggtaggaggatggtaatgcaaggggtcctagcatgtccagtatccaacaattcaacaatagcaagccaatgtcaatcttactattttaataactcattagggttcatcggggcagaatctagaatctattatctcaatggtaacatttatctttatcagagaagctcgagttggtggcctcacccccaaatctacctgcttgattctagaattgcaagtccgggtactcagaccattgactcaggtgtcaatctcaaaatgttaaatgtcactgtgattacacgaccatcatctggtttttgtaatagtcagtcacgatgccctaatgattgcttattcggggtctattcggatatctggcctcttagccttacctcagatagcatattcgcattcacaatgtatttacaggggaagacaacacgtattgacccggcttgggcgctattctccaatcatgcaattgggcatgaggctcgtctgtttaataaggaagttagtgctgcttattctaccaccacttgtttttcggacaccatccaaaatcaggtgtattgcctgagtatacttgaggtcagaagtgagctcttgggagcattcaaaatagtaccattcctctaccgcgtcttgtaggcatccattcagccaaaaaacttgagtgaccatgagattgacacctgatccccctcaaagacacctatctaaattactgttctagacccatgattaggtaccttcttaatcaatcatttggtttttaattaaaaatggaaaaatggacctagttccaagagagggctggaacccattagggtggggaaggattgctttgctccttgactcacactcacgtacactcgatcagacttctgttaaaaaggaaaccttctcaaactcgccccacgatgtccaatcaggcagctgagattatactacctagcttccatctagaatcacccttaatcgagaataagtgcttctattatatgcaattacttggtctcgtgttgccacatgatcactggagatggagggcattcgttaactttacagtggatcaggtgcaccttaaaaatcgtaatccccgcttaatggcccacatcgactacactaaagatagattgaggactcatggtgtcttaggtttccaccagactcagacaagtttgagccgttatcgtgttttgctccatcctgaaaccttaccttggctgtcagccatgggaggatgcatcaatcaggtgcctaaagcatggcggaacaccctgaaatcgatcgagcacagtgtaaagcaggaggcacctcaactaaagctactcatggagagaacctcattaaaattaactggggtaccttacttgttctctaattgcaatcccgggaaaaccaaagcaggaactatacctgtcctaagtgagatggcatcggaactcttgtcaaatcctatctcccaattccaatcaacatggggatgtgctgcttcggggtggcaccatgtagtcagtatcatgaggcttcagcaatatcaaagaaggacaggtaaggaggaaaaagcaatcactgaagttcagtatggcacagacacctgtctcattaacgcagactacaccgttgttttttccacacagaaccgtatcataacggtcttgcctttcgatgttgtcctcatgatgcaagacctgctcgaatcccgacggaatgtcctgttctgtgcccgctttatgtatcccagaagccaacttcatgagaggataagtacaatattagcccttggagaccaattggggaggaaagcaccccaagtcctgtatgattttgtagcaacccttgagtcatttgcatacgcagcggttcaacttcatgacaacaatcctacctacggtggggccttctttgaattcaacatccaagagttagaatcgattctgtcccctgcacttagtaaggatcaggtcaacttctacataagtcaagttgtctcagcgtacagtaaccttcctccatccgaatcggcagagctgctgtgcctgttacgcctgtggggtcatcccttgctaaacagccttgatgcagcaaagaaagtcagggagtctatgtgcgccgggaaggttctcgattacaacgccattcgacttgtcttgtctttttatcatacgttgctaatcaatgggtaccggaagaaacacaagggtcgctggccaaatgtgaatcaacattcacttctcaacccgatagtgaggcagctttattttgatcaggaggagatcccacactctgttgcccttgagcactatttggatgtttcaatgatagaatttgaaaaaacttttgaagtggaactatctgacagcctaagcatcttcctgaaggataagtcgatagctttggataagcaagaatggtatagtggttttgtctcagaagtgactccgaagcacctgcgaatgtcccgtcatgatcgcaagtctaccaataggctcctgttagccttcattaactcccctgaattcgatgttaaggaagagcttaaatacttgactacgggtgagtacgccactgacccaaatttcaatgtctcatactcacttaaagagaaggaggtaaagaaagaagggcgcattttcgcaaaaatgtcacaaaagatgagagcgtgccaggttatttgtgaagaattgctagcacatcatgtggctcctttgtttaaagagaatggtgttactcaatcagagctatccctgacaaaaaatttgttggctattagccaactgagttacaactcgatggccgctaaggttcgattgctgcggccaggggacaagttcactgctgcacactatatgaccacagacctaaaaaagtactgtcttaattggcggcaccagtcagtcaaactgttcgccagaagcctggatcgactgtttgggttagaccatgctttttcttggatacatgtccgtctcaccaacagcactatgtacgttgctgacccctttaatccaccagactcagatgcatgcacaaatttagacgacaataagaataccgggatctttattataagtgcacgaggtggtatagaaggcctccaacaaaagctatggactggcatatcaattgcaattgcccaagcggcagcggccctcgaaggcttacgaattgctgctactctgcagggggataaccaagttttggcgattacaaaggaattcatgaccccagtcccagaagatgtaatccatgagcagctatctgaggcgatgtctcgatacaaaaggactttcacatacctcaattatttaatgggacatcagttgaaggataaggaaaccatccaatctagtgatttctttgtttactccaaaagaatcttcttcaatggatcaatcttaagtcaatgcctcaagaacttcagtaaactcactactaatgccactacccttgctgagaatactgtggccggctgcagtgacatctcttcatgcattgcccgttgtgtggaaaacgggttgccaaaggatgccgcatacatccagaatataatcatgactcggcttcaactattgctagatcattactattcaatgcatggcggcataaactcagagttagagcagccaacgttaagtatctctgttcgaaacgcaacctacttaccatctcaactaggcggttacaatcatttaaatatgactcgactattctgccgcaatatcggcgacccgcttaccagttcttgggcagagtcaaaaagactaatggatgttggtctcctcagtcgtaagttcttggaggggatattatggagacccccgggaagtgggacgttttcaacactcatgcttgatccgttcgcacttaacattgattacctgaggccgccagagacaattatccgaaaacacacccaaaaagtcttattgcaagattgtccaaaccccctattagcaggtgtcgttgacccaaactacaaccaagaattagagctgttagctcagttcttgcttgatcgggaaaccgttattcccagggctgcccatgccatctttgagttgtctgtcttggggaggaaaaaacatatacaaggattggtagatactacaaaaacaattattcagtgctcattggaaagacagccattgtcctggaggaaagttgagaacattgttacctacaacgcgcagtatttcctcggggccacccaacaggctgacactaatgtctcagaagggcagtgggtgatgccaggtaacttcaagaagcttgtgtcccttgacgattgctcggtcacgttgtctaccgtatcacggcgcatatcgtgggccaatctactgaactggagagctatagacggtttggaaaccccggatgtgatagagagtatcgatggccgccttgtacaatcatccaatcaatgtggcctatgtaatcaagggttggggtcctactcctggttcttcttgccctctgggtgtgtgttcgaccgtccacaagattcccgggtggttccaaagatgccatatgtggggtccaaaacagatgagagacagactgcatcagtgcaagctatacaaggatccacttgtcacctcagggcggcattgaggcttgtatcactctacctatgggcctatggggattctgacatatcatggctagaagctgcgacactggctcaaacacggtgcaacgtttctcttgatgacttgcgaatcttgagccctctcccttcttcggcgaatttacaccacagattaaatgacggggtaacacaggttaaattcatgcccgccacatcgagccgagtgtcaaagttcgtccaaatttgcaatgacaaccagaatcttatccgtgacgatggaagtgttgattccaatatgatttatcaacaggttatgatattagggcttggggagattgaatgcttgttagctgacccaattgatacaaacccagaacaattgattcttcatctacactctgataattcttgctgtctccgggagatgccaacgaccggctttgtaccagctctaggactgaccccatgtttaactgtcccaaagcacaatccttacatatatgatgatagcccaatacctggtgatttggatcagaggctcattcagaccaaatttttcatgggttctgacaatttggataatcttgatatctaccaacagcgagctttactgagtaggtgtgtggcttatgatgttatccaatcgatctttgcttgtgatgcaccagtctctcagaagaatgacgcaatccttcacactgactatcatgagaattggatctcagagttccgatggggtgaccctcgtattatccaagtaacggcaggctacgagttaattctgttccttgcataccagctttattatctcagagtgagaggtgatcgtgcaatcctgtgttatgttgacaggatactcaataggatggtatcttccaatctaggcagtctcatccagacactctctcatccagagattaggaggagattctcgttgagtgatcaagggttccttgttgagagggaactagagccaagtaagcccttggttaaacaagcggttatgttcttgagggactcagtccgctgcgctctagctactatcaaggcaggaattgagcctgagatctcccgaggtggctgtactcaggatgagctaagctttactcttaagcacttactgtgtcggcgtctctgtgtaatcgctctcatgcattcagaggcaaagaacttggttaaggttagaaaccttcctgtagaagagaaaaccgccttactgtatcagatgttggtcactgaggccaatgctaggaaatcaggatctgctagcattatcataaacctagtatcggcaccccagtgggatattcatacaccagcattgtattttgtgtcaaagaaaatgttagggatgcttaagaggtcaaccacacccttggatataagtgacctctctgagagccagaatcccgcaccggcagagctgaatgatgttcctgatcacatggcagaagaatttccctgtttgtttagtagttataacgctacatatgaagacacaatcacttacaatccaatgactgaaaaactcgccttgcacttggacaatagttccaccccatccagagcacttggtcgtcactacatcctgcggcctcttgggctttactcatctgcatggtaccggtctgcagcactactagcatcaggggccctaaatgggttgcctgaggggtcaagcctgtatctaggagaagggtacgggaccaccatgactctgcttgagcccgttgtcaagtcttcaactgtttactaccacacattgtttgacccaacccggaatccttcacagcggaactataaaccagaaccacgggtattcacggattctatttggtacaaggatgatttcacacggccacctggtggtattatcaacctgtggggtgaagatatacgtcagagtgatatcacacagaaagacacggtcaacttcatactatctcagatcccgccaaagtcacttaagttgatacacgttgatattgaattctcaccagactccgatgtacggacactactttctggctattctcattgtgcattattggcctactggctattgcaacctggagggcgatttgcggttagggttttcttaagtgaccatgtcatagtaaacttggtcactgcaattctgtctgcttttgactctaatttggtgtgcattgcatcaggattgacacacaaggatgatggggcaggttatatttgcgcaaagaagcttgcaaatgttgaggcttcaaggattgaatactacctgaggatggtccatggttgtgttgactcattaaagatccctcatcaattaggaatcattaaatgggccgagggtgaggtgtcccaacttaccagaaaggcagatgatgaaataaattggcggttaggtgatccggttaccagatcatttgatccagtttctgagctaatcattgcacgaacaggggggtctgtattgatggaatacggggcttttactaacctcaggtgtgcgaacttggcagatacatacaaacttctggcttcaattgtagagaccaccttaatggaaataagggttgaacaagaccagttggaagataattcgaggaggcaaatccaaatagtccccgcttttaacacgagatctgggggaaggatccgtacactgattgagtgtgctcagctgcagattatagatgttatttgtgtaaacatagatcacctctttcctagacaccgacatgttcttgtcacgcaacttacctaccagtcggtgtgccttggggacttgattgaaggcccccaaattaagacgtatctgagggccagaaagtggatccaacgtcggggactcaatgagacagttaaccatatcatcactggacaagtgtcacggaataaagcaagggattttttcaagaggcgcctgaagttggttggcttttcactctgcggtggttggagctacctctcactttaactgttcaagttgttgattattatgaataatcggagtcggaatcgtaaatagtaagccacaaagtcgtgaataaacaatgattgcattagtatttaataaaaaatatgtcttttatttcgt

SEQ ID NO: 80 - DNA, 15054 bp, Artificial Sequence
  Avian paramyxovirus 4 isolate APMV-4/Egyptian goose/South Africa/N1468/2010, complete genome (GenBank JX133079.1)
acgaaaaagaagaataaaaggcagaagccttttaaaaggaaccctgggctgtcgtaggtgtgggaaggttgtattccgagcgcgcctccgaggcatctactctacacctatcacaatggctggtgtcttctcccaatatgagaggtttgtggacaatcaatcccaagtgtcaaggaaggatcatcggtccctggcagggggatgccttaaagtcaacattcctatgcttgtcactgcatctgaagatcccaccactcgttggcaactagcgtgtttatctttgaggctcttgatctccaactcatcaaccagtgctatccgccagggggcaatactgactctcatgtcactaccatcacaaaatatgagagcaacggcagctattgctggttccacaaatgcagctgttatcaacactatggaagtcttgagtgtcaatgactggaccccatccttcgaccctaggagcggtctctctgaagaggatgctcaggttttcagagacatggcaaaggacctgccccctcagttcacctccggatcaccctttacatcagcattggcggaggggtttaccccagaagacacccacgacctaatggaggccttgactagtgtgctgatacagatctggatcctggtggctaaggccatgaccaacattgatggctctggagaggccaatgagagacgtcttgcaaagtacatccagaagggacaactcaatcgccagtttgcaattggtaatcctgctcgtctgataatccaacagacgatcaaaagctccttaactgtccgcagattcttggtctctgaacttcgtgcatcacgaggtgcggtgaaagaaggatccccttactatgcagctgttggggacatccacgcttacatctttaacgcaggactgacaccattcttgactaccttaagatatgggatcggcaccaagtatgctgcagttgcactcagtgtgttcgctgcagacattgcaaaattaaagagcctacttaccctatatcaagacaagggtgtggaggctggatacatggcactccttgaagatccagactccatgcactttgcacctggaaacttcccacacatgtactcctacgcgatgggggtggcttcttaccatgaccccagcatgcgccagtaccaatatgctaggaggttcctcagccgacctttctacttgctagggagggacatggccgccaagaacacaggcacgctggatgagcaactggcaaaggaactgcaagtgtcagaaagagaccgcgccgcattgtccgctgcgattcagtcagcaatagaggggggagaatccgacgacttcccactgtcgggatccatgccggctctctccgacaatgcgcaaccagttaccccaagaacccaacagtcccagccctcccctccccaatcatcaagcatgtctcaatcagcacccaagaccccggactaccagcctgattttgaactgtaggctgcatcagtgcaccaacagcaggccaaagggaccaccctcctccccacacatcccacccaatcacccgctgagacccaatccaacaccccagcatccccctcatttaattaaaaactgaccaatagggtggggaaggagagctgttggctatcgccaagatcgtgcagcgatggattttaccgatattgatgctgtcaactcattaattgaatcatcatcagcaatcatagattccatacagcatggagggctgcaaccatcaggtactgttggcctatcgcaaatccccaaggggataaccagcgctttaaccaaggcctgggaggctgagacagcaactgctggctacggggacacccaacacaaatctgacagtccggaggatcatcaggccaacgacacagactcccccgaagacacaggcaccaaccagaccatccaggaagccaacatcgtcgaaacaccccaccccgaagttctatcggcagccaaagccagactcaagaggcccaaggcagggaaggacacccacgacaatccccctgcgcaacccgatccccttttaaaggggggccccctgagcccacaaccagcagcaccgtgggtgcaaaattcacccattcatggaggtcccggcaccgccgatccccgcccatcacaaactcaggatcattccctcaccggagagagatggcaatcgtcaccgataaagcaaccggagacattgaactggtggaatggtgcaacccggggtgcacagcaatccgaactgaaccaaccagactcgactgtgtatgcggatactgccccaccatctgcagcctctgcatgtatgacgactgatcaggtacaactattaatgaaggaggttgccgatatgaaatcactccttcaggcactagtgaggaacctagctgtcctgcctcaactaaggaacgaggttgcagcaatcaggacatcacaggctatgatagaggggacactcaattcaatcaagattctcgaccctgggaattatcaagaatcatcactgaacagttggttcaaaccacgccaagatcacgcggttgctgtgtccggaccagggaatccattgaccatgccaactccaatccaagacaacaccatattcctggatgaactggcaagacctcatcctagtttggtcaatccgtccccgcccactaccaacactaatgttgaccttggcccacagaagcaggctgcgatagcttatatctcagcaaaatgcaaggatcaagggagacgagatcagctctcaaagctcatcgagcgagcaaccaccttgagtgagatcaacaaagtcaaaagacaggcccttggcctctagaccactcgaccacccccagtaatgaacacaacaataatcagaacctccctaaaccacacggccaacccagcacaccatccacaccgcccaccactatcccccgccaaaaactccgctgcagccgatttattcaaaagaagccacttgatatgacttatcaaccgcaaggtagggtggggaaggtgctttgcctgcaagagggctccctcatcttcagacacgtacccgccaacccaccagtgacgcaatggcagacatggacactgtatatatcaatctgatggcagatgatccaacccaccaaaaagaactgctgtccttccctctcattccagtgactggtcccgacgggaaaaaggaactccaacaccaggttcggactcaatccttgctcgcctcagacaagcaaactgagaggttcatcttcctcaacacttacgggtttatctatgacactacaccggacaagacaactttttccaccccagagcatatcaatcagcccaagagaacgatggtgagtgctgcaatgatgaccatcggcctggtccccgccaatatacccttgaacgaactaacagctactgtgtttggcctgaaagtaagagtgaggaagagtgcgagatatcgagaggtggtctggtatcagtgcaaccctgtaccagccctgcttgcagccaccaggtttggtcgccaaggaggtctcgaatcgagcactggagtcagtgtgaaggcccccgagaagatagattgcgagaaggattatacttactacccttatttcctatctgtgtgctacatcgccacttctaacctgttcaaggtaccaaaaatggttgctaatgcgaccaacagtcaattataccacctgacgatgcaggtcacatttgcctttccaaaaaacattcccccagctaaccagaaactcctgacacaagtggatgaaggattcgagggcactgtggactgccattttgggaacatgctgaaaaaggatcggaaagggaatatgaggacattgtcgcaggcggcagataaggtccgacggatgaacatccttgttggtatctttgacttgcatgggccgacactcttcctggagtataccgggaaactaacgaaagctctgttagggttcatgtctaccagccgaacagcaatcatccccatatctcagctcaatcctatgctgagtcaactcatgtggagcagtgatgctcagatagtaaaattaagagtggtcataactacatccaaacgcggcccatgcgggggtgagcaggaatatgtgctggaccccaaattcacagttaaaaaagaaaaagcccgactcaaccctttcaagaaggcagcttaatgatcaaatctgcaggatctcaggaatcagaccactctatactatctactgatcaatagatatgtagctatacagttgatgaacctatgaagaatcaattagcaaaccgaatccttgctagggtggggaaggaattgattgggtgtctaaacaaaagcacttctttgcacctactcaccacaaaacaatcataatgaggttatcacgaacaatcctggccctgattctcggcgcacttaccggctatttaatggatgcccactccaccactgtgaatgagagaccaaagtctgaggggattaggggtgaccttataccaggtgcaggaatctttgtaactcaaatccggcaactacagatctaccaacaatctgggtatcatgaccttgtcatcaggttattacctcttttaccggcagaactcaatgattgccaaagggaagttgtcacagagtacaacaatacagtatcacagctgttgcagcctatcaaaactaacctggataccttattggctgatggtggcacaagggatgccgatatacagccgcggttcattggggcgataatagccacaggtgccctggcagtggctacggtagctgaggtgactgcagcccaagcactatctcagtcgaaaacgaacgctcaaaatattctcaagttgagagatagtattcaggccaccaaccaggcagtttttgaaatttcacaaggacttgaggcaactgcaactgtactatcaaaactgcaagctgagctcaatgagaacattatcccaagtctgaacaacttgtcctgtgctgccatggggaatcgccttggtgtatcactatcactctacttgaccctaatgactaccctatttggggaccagatcacaaacccagtgctgacaccaatctcctatagcactttatcggcaatggcaggtggtcacattggcccggtgatgagtaaaatattagccggatctgtcacaagtcagttgggggcagaacagttgattgctagcggcttaatacaatcacaggtagtaggttatgattcccaatatcaattattggttatcagggtcaaccttgtacggattcaagaggtccagaatacgagggtcgtatcactaagaacactagcggtcaatagggatggtggactttatagagcccaggtgcctcccgaggtagtcgaacggtctggcattgcagagcgattttatgcagatgattgtgttcttactacaactgattacatttgctcctcgatccgatcttctcggcttaatccagagttagtcaaatgtctcagtggggcacttgattcatgcacatttgagagggaaagtgcattattgtcaacccctttctttgtatacaacaaggcagttgtcgcaaattgtaaagcggcaacatgtagatgcaataaaccgccgtctattattgcccaatactctgcatcagctctggtcaccatcaccaccgacacctgcgccgaccttgaaattgagggctatcgcttcaatatacagactgaatccaactcatgggttgcaccaaacttcactgtctcgacttcacagattgtatcagttgatccaatagacatctcctctgacattgctaaaatcaacagttccatcgaggctgcaagagagcagctggaactaagcaaccagatcctttcccgaattaacccacgaattgtgaatgatgaatcattgatagctattatcgtgacaattgttgtgcttagtctcctcgtaatcggtctgattgttgttctcggtgtgatgtataagaatcttaaaaaagtccaacgagctcaagctgccatgatgatgcagcagatgagctcatcacagcccgtgaccactaaattagggacgcccttctaggataataatcatatcactctactcaatgatgagcaagacgtaccaatcatcaatgattgtgtcacaaggccggtagggaatgcaccgaatttctcccctttctttttaattaaaaacatttgtagtgaggatgagaaggggaaaatgtttggtagggtggggaaggtagccaattcctgcctattaggccgaccgtatcaaaagaactcaacagaagtccagatacaaggtaacatggagggcagccgtgataatcttacagtggatgatgaattaaagacaacgtggaggttagcttatagagttgtgtcccttctattgatggtgagcgctttgataatctctatagtaatcctgacgagagataacagccaaagcgtaatcacggcgatcaaccagtcatctgaagctgactccaagtggcaaacgggaatagaagggaaaatcacctccattatgactgatacgctcgataccaggaatgcagcccttctccacattccactccagctcaactcgcttgaggcgaacctattgtccgcccttgggggcaacacaggaattggccccggagatatagagcactgccgttaccctgttcatgacaccgcttacctgcatggagttaatcgattactcatcaaccagacagctgattatacagcagaaggccccctagatcatgtgaacttcattccagccccggttacgaccactggatgcacaaggataccatccttttccgtgtcatcgtccatttggtgctatacacacaacgtgattgaaaccggttgcaatgaccactcaggtagtaaccaatatatcagcatgggagtcattaagagagcgggcaacggcctaccttacttctcaacagttgtaagtaagtatctgactgatgggttgaataggaaaagctgttctgtagctgccggatctgggcattgctacctcctttgcagcttggtgtcggagcccgaatctgatgactatgtgtcacctgatcctacaccgatgaggttaggggtgctaacgtgggatgggtcttacactgagcaggtggtacccgaaagaatattcaagaacatatggagtgcaaactacccaggagtagggtcaggtgctatagtaggaaataaggtgttattcccattttacggcggagtgagtaatggatcgaccccggaggtgatgaataggggaagatattactacatccaggatccaaatgactattgccctgacccgctgcaagatcagatcttaagggcggaacaatcgtattacccaactcgattcggtaggaggatggtgatgcaaggggtcctagcatgtccagtatccaacaattcaacaatagcaagccaatgtcaatcttactattttaataactcattagggttcattggggcagaatctaggatctattacctcaatgataacatttatctttaccagagaagctcgagctggtggcctcacccccagatttacctgcttgattctaggattgcaagtccgggtactcagaacattgactcaggtgtcaatctcaagatgttaaatgtcactgtaattacacgaccatcatctggtttttgtaatagtcagtcacgatgccctaatgactgcttattcggggtctactcggatatctggcctcttagccttacctcagatagcatattcgcattcacaatgtatttacaggggaagacaacacgtattgacccggcttgggcgctattctccaatcatgcgattgggcatgaggctcgtctgtttaataagaaggttagtgctgcttattctaccaccacttgtttttcggacaccgtccaaaatcaggtgtattgcctgagtatacttgaggtcaggagtgagctcttgggagcattcaaaatagtaccattcctctatcgcgtcttgtaggcatccattcagccagaaaacttgagtgaccatgatattaacacctgatccccctcaaagacacctatctaaattactgttctagactcatgattaggtaccttcttaatcaatcatttggtttttaattaaaaatgaaaaaataggcctagttccaagagagggctggaacccattagggtggggaaggattgctttgctccttgactcacacacacgtacactcgatcagactcctgtttaaaaggaatccttctcaaactcgccccacgatgtccaatcaggcggctgagattatactacccaccttccatctagaatcacccttaatcgaaaataagtgcttctattatatgcaattacttggtctcgtgttgccacatgatcactggagatggagggcattcgttaactttacagtggatcaggtgcaccttaaaaatcgtaatccccgcttgatggcccacatcgactacactaaggatagattaaggactcatggtgtcttaggtttccaccagactcagacaagtttgagccgttatcgtgttttgctccatcctgaaaccttatcttggctatcagccatggggggatgcatcaatcaggttcctaaagcatggcggaacactctgaaatcgatcgagcacagtgtaaagcaggaggcacctcaactaaagctactcatggagagaacctcattaaaattaactggagtaccttacttgttctctaattgcaatcccgggaaaaccacagcaggtactatgcctgtcctaagtgagatggcatcggaactcttgtcgaatcctatctcccaattccaatcaacatgggggtgtgctgcttcggggtggcaccatgtagtcagtatcatgaggctccaacaataccaaagaaggacaggtaaagaagagaaagcgatcactgaagttcagtatggcacagacacctgtctcattaatgcagactacactgttgtgttttccacacagaaccgtatcataacagtcttgccttttgatgttgtcctcatgatgcaagacctgctcgaatcccgacggaatgtcctgttctgtgcccgctttatgtatcccagaagccaacttcatgagaggataagtacaatattagctcttggagaccaactggggagaaaagcaccccaagtcctgtatgatttcgtagcaacccttgagtcatttgcatacgcggctgttcaacttcatgacaacaatcctacctacggtggggccttctttgaattcaatatccaagagttagaatccattctgtcccctgcacttagtaaggatcaggtcaacttctacataaatcaagttgtctcagcgtacagtaaccttcccccatctgaatcggcagaattgctgtgcctgttacgcctgtggggtcaccccctgctaaacagccttgatgcagcaaagaaagtcagggagtctatgtgcgccgggaaggttctcgattacaacgccattcgacttgtcttgtctttttatcatacgttgctaatcaacggataccggaagaaacacaagggtcgctggccaaatgtgaatcaacattcactcctcaacccgatagtgaggcagctttattttgatcaggaggagatcccacactctgttgctcttgagcactatttggacgtctcaatggtagaatttgaaaaaacttttgaagtggaattatctgacagcctaagcatcttcctaaaggataagtcgatagctttggataagcaagagtggtacagtggttttgtctcagaagtgactccgaagcacctgcgaatgtcccgtcatgatcgcaagtctaccaataggctcctgttagccttcattaactcccctgaattcgatgttaaggaagagcttaaatacttgactacgggtgagtacgccactgacccaaatttcaatgtctcatactcacttaaagagaaggaagtaaagaaagaggggcgcattttcgcaaaaatgtcacaaaagatgagagcatgccaggttatttgtgaagaattgctagcacatcatgtggctcctttgtttaaagagaatggtgttactcaatcagagctatccctgacaaaaaatttgttggctattagccaactgagttacaactcgatggccgctaaggtgcgattgctgagaccaggggacaagttcactgctgcacactatatgaccacagacctaaaaaagtactgtcttaattggcggcaccagtcagtcaaactgttcgccagaagcctggatcgactgtttgggttagaccatgctttttcttggatacatgtccgcctcaccaacagcactatgtacgttgctgaccccttcaatccaccagactcagatgcatgcattaatttagacgacaataagaacactgggatttttattataagtgcacgaggtggtatagaaggcctccaacaaaaactatggactggcatatcaattgcaattgcccaagcggcagcggccctcgaaggcttacgaattgctgctactctgcagggggataaccaagttttggcgattacaaaggaattcatgaccccagtcccagaggatgtaatccatgagcagctatctgaggcgatgtctcgatacaaaaggactttcacatacctcaattatttaatgggacatcaattgaaggataaggaaaccatccaatccagtgatttctttgtctattccaaaagaatcttcttcaatggatcaatcttaagtcaatgcctcaagaacttcagtaaactcactactaatgccactacccttgctgagaatactgtggccggctgcagtgacatctcttcatgcattgcccgttgtgtggaaaacgggttgcctaaggatgccgcatatatccagaatataatcatgactcggcttcaattattgctagatcattactattcaatgcatggcggcataaactcagaattagagcagccaactttaagtatctctgttcgaaacgcaacctacttaccatctcaactaggcggttacaatcatctaaatatgacccgactattctgccgcaatatcggcgacccgcttaccagttcttgggcggagtcaaaaagactaatggatgttggtctcctcagtcgtaagttcttggaggggatattatggagacccccgggaagtgggacgttttcaacactcatgcttgacccgttcgcacttaacattgattacctgaggccgccagaaacaattatccgaaaacacacccaaaaagtcttgttgcaagattgcccaaaccccctattagcaggtgtcgttgacccaaactacaaccaagaattagagctgttagctcagttcttgcttgatcgggagaccgttattcccagggctgcccatgccatctttgagttgtctgtcttggggaggaaaaaacatatacaaggattggtggacactacaaaaacaattattcagtgctcattggaaagacagccattgtcctggaggaaagttgagaacattgttacctacaacgcgcagtatttcctcggggccacccaacaggctgatactaatgtctcagaagggcagtgggtgatgccaggtaacttcaagaagcttgtgtcccttgacgattgctcggtcacgttgtctactgtatcacggcgcatatcgtgggccaatctactgaactggagagctatagatggtttggaaaccccggatgtgatagagagtattgatggccgccttgtacaatcatcaaatcaatgtggcctatgtaatcaagggttggggtcctactcttggttcttcttgccctctgggtgtgtgttcgaccgtccacaagattcccgggtagttccaaagatgccatacgtggggtccaaaacagatgagagacagactgcatcagtgcaagctatacaaggatccacttgtcacctcagggcagcattgaggcttgtatcactctacttatgggcttatggagattctgacatatcatggctagaagctgcgacactggctcaaacacggtgcaatgtttctcttgatgacttgcgaatcttgagccctctcccttcttcggcgaatttacaccacagattaaatgacggggtaacacaggttaaattcatgcccgccacatcgagccgagtgtcaaagttcgtccaaatttgcaatgacaaccaaaatcttatccgtgatgatgggagtgttgattccaatatgatttatcaacaggttatgatattagggcttggggagattgaatgcttgttagctgacccaattgatacaaacccagaacaattgattcttcatctacactctgataattcttgctgtctccgggagatgccaacgactggctttgtacctgctctaggactgaccccatgtttaactgtcccaaagcacaatccttacatttatgatgatagcccaatacctggtgatttggatcagaggctcattcagaccaaatttttcatgggttctgacaatttggataatcttgatatctaccaacagcgagctttactgagcaggtgtgtggcttatgatgttatccaatcgatctttgcctgtgatgcaccagtctctcagaagaatgacgcaatccttcacactgactatcatgagaattggatctcagagttccgatggggtgaccctcgtattatccaagtaacggcaggctacgagttaattctgttccttgcataccagctttattatctcagagtgaggggtgaccgtgcaatcctgtgttatattgacaggatactcaataggatggtatcttccaatctaggcagtctcatccagacactctctcatccagagattaggaggagattctcattgagtgatcaagggttccttgttgaaagggaattagagccaggtaagcccttggttaagcaagcggttatgttcttgagggactcggtccgctgcgctttagcaactatcaaggcaggaattgagcctgagatctcccgaggtggctgtactcaggatgagctgagctttactcttaagcacttactatgccggcgtctctgtgtaatcgctctcatgcattcagaagcaaagaacttggttaaagtcagaaaccttcctgtagaggagaaaaccgccttactgtaccaaatgttggtcactgaggccaatgctaggaagtcaggatctgctagcattatcataaacctagtctcggcaccccagtgggacattcatacaccagcactgtattttgtgtcaaagaaaatgctagggatgcttaagaggtcaaccacacccttggatataagtgacctctccgagagccagaattccgcacctgcagagctgactgatgttcctggtcacatggcagaagagtttccctgtttgtttagtagttataacgccacatatgaagacacaattacttacaatccaacgactgaaaaactcgccttgcacttggacaacagttccaccccatccagagcacttggccgtcactacatcctgcggcctcttgggctttattcatccgcatggtaccggtctgcagcactactagcgtcaggggccttgaatgggttgcctgaggggtcaagcctgtatctaggagaagggtacgggaccaccatgactctgcttgagcccgttgtcaagtcttcaactgtttactaccatacattgtttgacccaacccggaatccttctcagcggaactataagccagaaccacgggtattcacggattctatttggtacaaggatgatttcacacggccacctggtggtattatcaacctgtggggtgaagatatacggcagagtgatatcacacagaaagacacggtcaacttcatactatctcagatcccgccaaaatcacttaagttgatacacgttgatattgaattctcaccagactccgatgtacggacactactatctggctattctcattgtgcactattagcctactggctattgcaacctggagggcgatttgcagttagggttttcttaagtgaccatatcatagtaaacttagtcactgcaattctgtctgcttttgactctaatttggtgtgcattgcatcaggattgacacacaaggatgatggggcaggttatatttgcgcaaagaagcttgcaaatgttgaggcttcaaggattgagcactacttgaggatggtccatggttgcgttgactcattaaagatccctcatcaattaggaatcattaaatgggccgagggtgaggtgtcccaacttaccagaaaggcggatgatgaaataaattggcggttaggcgatcctgttaccagatcatttgatccagtttctgagctaatcattgcacgaacaggggggtctgtattaatggaatacggggcttttactaacctcaggtgtgcgaacttggcagatacatacaagcttctggcttcaattgtagagaccaccctaatggaaataagggttgagcaagatcagttggaagataattcgaggagacaaatccaagtagtccccgctttcaacacgagatctgggggaaggatccgtacgctgattgagtgtgctcagctgcagattatagatgttatttgtgtaaacatagaccacctctttcctaaacaccgacatgttcttgtcacgcaacttacctaccagtcggtgtgccttggggacctgattgaaggcccccaaattaagacgtatctaagggccagaaagtggatccaacgtcagggactcaatgagacagttaaccatatcatcactggacaagtgtcacggaataaagcaagggattttttcaagaggcgcttgaagttggttgggttttcactctgcggtggttggagctacctctcactttagctgttcaggttgtcgattattatgaataatcggagtcggaatcgcaaataggaagccacaaagttgtggagaaacaatgattgcattagtatttaataaaaaatatgtcttttatttcgt

SEQ ID NO: 81 - DNA, 15054 bp, Artificial Sequence
  Avian paramyxovirus 4 strain APMV4/duck/China/G302/2012, complete genome (GenBank KC439346.1)
acgaaaaagaagaataaaaggcagaagccttttaaaaggaaccctgggctgtcgtaggtgtgggaaggttgtattccgagtgcgccttcgaggcatctactctacacctatcacaatggctggtgtcttctcccagtatgagaggtttgtggacaatcaatcccaagtgtcaaggaaggatcatcgttccctggcagggggatgcctaaaagtcaacatccctatgcttgtcactgcatctgaagatcccaccactcgttggcaactagcatgtttatccttaaggctcttggtctccaactcatcaaccagtgctatccgccagggggcgatactgactctcatgtcactaccatcacaaaatatgagagcaacggcagctattgctggttccacaaatgcggctgttatcaacactatggaagtcttgagtgtcaacgactggaccccatccttcgaccccaggagcggtctctctgaagaggatgctcaggttttcagagacatggcaagggacctgccccctcagttcacctccgggtcaccctttacatcggcattggcggaggggtttaccccggaggacacccacgacctaatggaggccctgaccagtgtgctgatacagatctggatcctggtggctaaggccatgaccaacattgatggctctggggaagccaatgagagacgtcttgcaaagtacatccagaagggacagcttaatcgccagtttgcaattggtaatcctgctcgtctgataatccaacagacgatcaaaagctccttaactgtccgcaggttcttggtctctgagcttcgtgcatcacgaggtgcggtgaaagaaggatccccttactatgcggctgttggggatatccacgcttacatctttaacgcaggactgacaccattcttgactaccttaagatacgggataggcaccaaatatgctgctgttgcactcagtgtgttcgctgcagacattgcaaaattaaagagtctacttaccctataccaggacaagggtgtggaggccggatacatggcactcctcgaagatccagactctatgcactttgcgcctggaaacttcccacacatgtactcctacgcgatgggggtggcttcttaccatgaccccagcatgcgccagtaccaatatgctaggaggttcctcagccgtcctttctacttgctagggagggacatggctgccaagaacacaggcacgctggatgagcaactggcaaaggaactacaagtgtcagaaagagaccgtgccgcattgtccgctgcgattcaatcagcaatggaggggggagaatctgacgacttcccactatcgggatccatgccggctctctccgacaatgcgcaaccagttaccccaagaactcaacagtcccagctctcccctccccaatcatcaagcatgtctcaatcagcgcccaggaccccggactaccagcctgattttgaactgtaggctgcatccacgcaccaacagcaggccaaagaaaccacccccctcctcacacatcccacccaatcacccgccaagacccaatccaacaccccagcatccccctcatttaattaaaaactgaccaatagggtggggaaggagagttattggctattgccaagttcgtgcagcaatggattttaccgatattgatgctgtcaactcattaattgaatcatcatcagcaatcatagattccatacagcatggagggctgcaaccatcaggcactgtcggcctatcacaaatcccaaaggggataaccagcgccttaaccaaggcctgggaggccgaggcagcaactgctggcaacggggacacccaacacaaatctgacagtccggaagaccatcaggccaacgacgcagactcccccgaagacacaggcaccaaccagaccatccaagaagccaatatcgttgaaacaccccaccccgaagtgctatcggcagccaaagccagactcaagaggcccaagacagggagggacacccacgacaatccctctgcgcaacctgatcatcttttaaaggggggccccctgagcccacaaccagcggcaccgtgggtgaaagatccatccattcatggaggtcccggcaccgccgatccccgcccatcacaaactcaggatcattccctcaccggagagagatggcaatcgtcaccgacaaagcaaccggagacatcgaactggtggaatggtgcaacccggggtgcacagctatccgagctgaaccaaccagactcgactgtgtatgcggacactgccccaccatctgcagcctctgcatgtatgacgactgatcaggtacaactattaatgaaggaggttgccgacatgaaatcactccttcaggcactagtgaggaacctagctgtcctgcctcaactaaggaatgaggttgcagcaatcaggacatcacaggccatgatagaggggacactcaattcaatcaagattctcgaccctgggaattatcaagaatcatcactaaacagttggttcaaaccacgccaagatcacgcggttgttgtgtccggaccagggaatccattggccatgccaaccccgatccaagacaacaccatattcctagatgaactggcaagacctcatcctagtttggtcaatccgtccccgcccgctaccaacaccaatgctgatcttggcccacagaagcaggctgcgatagcttatatctcagcaaaatgcaaggatcaagggaaacgagaccagctctcaaagctcatcgagcgagcaaccaccctgagcgagatcaacaaagtcaaaagacaggcccttggcctctagaccactcgaccacccccagtgatgaatacaacaataatcagaacctccctaaaccacatggccaacccagcgcaccatccacaccacctattactacccttcgccagaaactccgccgcagccgatttattcaaaagaagccactcgatatgacttagcaaccgcaagatagggtggggaaggtgctttacctgcaagagggctccctcatcttcagacacgcacccgccaacccaccagtgacgcaatggcagacatggacactgtatatatcaatctgatggcagatgatccaacccaccaaaaagaactgctgtcctttcccctcattcccgtgactggtcctgacgggaaaaaggaactccaacaccaggtccggactcaatccttgctcgcctcagacaagcaaactgagaggttcatcttcctcaacacttacgggtttatctatgacactacaccggacaagacaactttttctaccccagagcatatcaatcaacccaagagaacgatggtgagtgctgcaatgatgaccatcggcctggtccccgccaatatacccttgaacgaactaacagctactgtgtttggcctgaaaataagagtgaggaagagtgcgagatatcgagaggtggtctggtaccagtgcaaccctgtaccagccctgcttgcagccacaaggtttggtcgccaaggaggtctcgaatcgagcactggagttagtgtaagggcccccgagaagatagactgcgagaaggattatacttactacccttatttcctatctgtgtgctacatcgccacttccaacctgttcaaggtaccaaaaatggtcgctaatgcgaccaacagtcaattataccacctgaccatgcagatcacatttgcctttccaaaaaacatccccccagctaaccagaaactcctgacactagtggatgaaggattcgagggcactgtggactgccattttgggaacatgctgaaaaaggatcggaaagggaacatgaggacactgtcgcaggcggcagacaaggtcagacggatgaacatccttgttggtatctttgacttgcatgggccaacactcttcctggagtacaccgggaagctaacaaaagctctgttagggttcatgtctaccagccgaacagcaatcatccccatatctcagctcaatcctatgctgagtcaactcatgtggagcagtgatgcccagatagtaaaattaagagtggtcataactacatccaaacgcggcccatgcgggggtgagcaggagtatgtgctggatcccaaattcactgttaaaaaagagaaagcccgactcaaccctttcaagaaggcagcccaatgatcaaatctacaagatctcaggaatcagaccactctatactatccactgatcaatagacatgtagctatacagttgatgaacctatgaagaatcagttagaaaaccgaatccttactagggtggggaaggagttgattgggtgtctaaacaaaaacattcctttacacctcctcgccacgaaacaaccataatgaggttatcacgcacaatcctgaccttgattctcggcacacttactgattatttaatgggtgctcactccaccaatgtaactgagagaccaaagtctgaggggattaggggtgatcttacaccaggcgcaggtatctttgtaactcaagtccgacaactacagatctaccaacagtctgggtatcatgaccttgtcatcagattattacctcttctaccggcagaactcaatgattgtcaaagggaagttgtcacagagtacaacaatacggtatcacagctgttgcagcctatcaaaaccaacctggataccttactggctggtggtggcacaagggatgccgatatacagccgcggttcattggggcaatcatagccacaggtgccctggcggtggctacggtagctgaggtgactgcagcccaagcactatctcagtcgaaaacaaacgctcaaaatattctcaagttgagggatagtattcaggccaccaaccaggcagttttcgaaatttcacaaggactcgaggcaactgcaactgtgctatcaaaactgcaaactgagctcaatgagaacattatcccaagcctgaacaacttgtcctgtgctgccatgggtaatcgccttggtgtatcactatcactctacttgaccttaatgaccaccctatttggggaccagatcacaaacccagtgctgacaccgatctcctatagcactctatcggcaatggcaggtggtcatattggcccggtaatgagtaaaatattagccggatctatcacaagtcagttgggggcggaacagttgattgctagcggcttaatacagtcacaggtagtaggttatgattcccaataccaattattggttatcagggtcaaccttgtacggattcaagaggtccagaatacgagagtcgtatcactaagaacactagcagtcaatagggacggtggactctatagagcccaggtgcctcccgaggtagttgaacggtctggcattgcagaacgattttatgcagatgattgtgttcttactacaaccgattacatttgctcatcgatccgatcttctcggcttaatccagagttagttagatgtctcagtggggcacttgattcatgcacatttgagagggaaagtgcattattgtcaacccctttctttgtatacaacaaggcagttgtcgcaaattgtaaagcagcaacatgtagatgtaataaaccgccgtctattattgcccaatactctgcatcagctctggtcaccatcaccaccgacacctgtgccgacctcgaaattgagggttatcgcttcaacatacagactgaatccaactcatgggttgcaccaaacttcactgtctcgacttcacagattgtatcagttgatcccatagacatctcttctgacattgccaaaatcaacagttccatcgaggctgcaagagagcagctggaactaagcaaccagatcctttcccggatcaacccacgaatcgtgaatgatgaatcactgatagctattatcgtgacaattgttgtgcttagtcccctcgtaatcggtctgattgttgttctcggtgtgatgtataagaatcttaggaaagtccaacgagctcaagctgccatgatgatgcagcaaatgagctcatcacagcctgtgaccactaaattagggacgcctttctaggagaacaaccatatcactccactcaatgatgagcaagacgtaccaatcatcaatgattgtgtcacaaggccggttgggaatgcatcgaatctctcccctttctttttaattaaaaacatttgaagtgaagatgagaggggggaagtgtatggtagggtggggaaggcagccaattcctgcccattaggccgaccgtatcaaaaggattcaatagaagtctaggtacagggtaacatggagggcagccgcgataatcttacagtggatgatgaattaaagacaacatggaggttagcttatagagttgtgtctcttctattgatggtgagcgctttgataatctctatagtaatcctgacgagagataacagccaaagcataatcacggcgatcaaccagtcatctgacgcagactctaagtggcaaacgggaatagaagggaaaatcacctccattatggctgatacgctcgataccaggaatgcagttcttctccacattccactccagctcaacactcttgaggcgaacctattgtctgcccttgggggcaacacaggaattggccccggagatctagagcactgccgttaccctgttcatgacaccgcttacctgcatggagttaatcgattactcatcaatcagacagctgattatacagcagaaggccccctagatcatgtgaacttcattccagccccggttacgactactggatgcacaaggataccatccttttccgtgtcatcgtccatttggtgctatacacataacgtgattgaaaccggttgcaatgaccactcaggtagtaatcaatatatcagcatgggagtcattaagagagcgggcaacggcctaccttacttctcaacagttgtaagtaagtatctgactgatgggttgaataggaaaagctgttctgtggctgccggatctgggcattgctacctcctttgcagcttagtgtcggagcccgaacctgatgactatgtgtcacctgatcctacaccgatgaggttaggggtgctaacgtgggatggatcttacactgaacaggtggtacccgaaagaatattcaggaacatatggagtgcaaactacccaggagtagggtcaggtgctatagtaggaaataaggtgttattcccattttacggcggagtgaggaatggatcgaccccggaggtgatgaataggggaaggtactactacatccaggatccaaatgactattgccctgacccgctgcaagatcagatcttaagggcggaacaatcgtattacccaactcgattcggtaggaggatgataatgcagggggtcctagcatgtccagtatccaacaattcaacaatagcaagccaatgtcaatcttactattttaataactcattagggttcattggagcagaatctagaatctattacctcaatagtaacatttacctttatcagaggagctcgagctggtggcctcacccccagatttacctgcttgattctaggattgcaagtccgggtactcagaacattgactcaggtgtcaatctcaagatgttaaacgtcactgtgattacacgaccatcatctggtttttgtaatagtcagtcacgatgccctaatgactgcttattcggggtctactcggatatctggcctcttagccttacctcggatagcatattcgcgttcactatgtatttacaggggaagacaacacgtattgacccggcttgggcgctattctccaatcatgcgattgggcatgaggctcgtctgtttaataaggaggttagtgctgcttattctaccaccacttgttttttggacaccatccaaaaccaggtgtattgcctgagtatacttgaggtcaggagtgagctcttgggagcattcaaaatagtaccattcctctatcgtgtcttgtaggcatccattcggccaaaaaacttgagtgactatgaggttaacacttgatcccccttaaagacacctatctaaattactgtcctagacccatgattaggtaccttttaaatcaatcatttggtttttaattaaaaatgaaaaaatgggcctagtttcaagagagggctggaacccactagggtggggaaggattgctttgctccttgactcacacccacgtatactcgatctcacttctgtaaagaagggatccttctcaaactcgccccacaatgtccaatcaggcagctgagattatactacccaccttccatctagaatcacccttaatcgagaataagtgcttttattatatgcaattacttggtctcgtgttgccacatgatcattggagatggagggcattcgttaactttacagtggatcaggtgcaccttaaaaatcgtaatccccgcttaatggcccatatcgaccacactaaagatagattaaggactcatggtgtcttaggtttccaccagactcagacaagtttgagccgttatcgtgttttgctccatcctgaaaccttaccttggctatcagccatgggaggatgcatcaatcaggttcctaaagcatggcggaatactctgaaatcgatcgagcatagtgtaaagcaggaggcacctcaactaaagctactcatggagagaacctcattaaaattaactggagtaccttacttgttctctaattgcaatcccgggaaaaccacagcaggaactatgcctgtcctaagtgagatggcatcggaactcttgtcaaatcctatctcccaattccaatcaacatgggggtgtgctgcttcggggtggcaccatgtagtcagtatcatgaggctccaacaatatcaaagaaggacaggtaaggaagagaaagcaatcaccgaagttcagtatggcacagacacttgtctcattaacgcagactataccgttgttttttccacacagaaccgtgttataacggtcttgcccttcgatgttgtcctcatgatgcaagacctactcgaatcccgacggaatgttctgttctgtgcccgctttatgtatcccagaagccaacttcatgagaggataagtgcaatattagcccttggagaccaactggggagaaaagcaccccaagtcctgtatgatttcgtggcgaccctcgagtcatttgcatacgcagctgttcaacttcatgacaacaatcctacctacggtggggccttctttgaattcaatatccaagagttagaatctattctgtcccctgcacttagtaaggatcaggtcaacttctacataggtcaagttgtctcagcgtacagtaaccttcctccatctgaatcggcagaattgttgtgcctgctacgcctgtggggtcatcccttgctaaacagccttgatgcagcaaagaaagtcagggagtctatgtgtgccgggaaggttctcgattacaacgccattcgactcgtcttgtctttttaccatacattgttaatcaatgggtaccgaaagaaacacaagggtcgctggccaaatgtgaatcaacattcactcctcaacccgatagtgaggcagctctattttgatcaggaagagatcccacactctgttgcccttgagcactatttggatgtctcaatgatagaatttgaaaaaacttttgaagtggaactatctgacagcctaagcatcttcctgaaggataagtcgatagctttggataagcaagaatggtacagtggttttgtctcagaagtgactccgaagcacctacgaatgtctcgtcatgatcgcaagtctaccaataggctcctgttagctttcattaactcccctgaattcgacgttaaggaggagcttaagtacttgactacgggtgagtacgccactgacccaaatttcaatgtctcatactcacttaaagagaaggaagtaaaaaaagaagggcgcatattcgcaaaaatgtcacaaaagatgagagcatgccaggttatttgtgaagaattgctagcacatcatgtggctcctttgtttaaagagaatggtgttactcaatcagagctatccctgacaaaaaatttgttggctattagccaactgagttacaactcgatggctgctaaggtgcgattgctgaggccaggggacaagttcactgctgcacactatatgaccacagacctaaagaagtactgtctcaattggcggcaccagtcagtcaaactgttcgccagaagcctggatcgactgtttggattagaccatgcgttttcttggatacatgtccgtctcaccaacagcactatgtacgttgctgaccccttcaatccaccagactcagaggcatgcacagatttagacgacaataagaacaccgggatttttattataagtgcaagaggtggtatagaaggcctccaacaaaaattatggactggcatatcgattgcaattgcccaagcggcagcggccctcgaaggcttacgaattgctgctactctgcagggggataaccaagttttggcgattacgaaggaattcatgaccccagtcccagaggatgtaatccatgagcagctatctgaggcgatgtctcgatacaaaaggactttcacatacctcaattatttaatggggcatcagttgaaggataaagaaaccatccaatccagtgacttctttgtttattccaaaagaatcttcttcaatggatcgatcttaagtcaatgcctcaaaaacttcagtaaactcactactaatgccactacccttgctgagaatactgtggccggctgcagtgacatctcttcatgcattgcccgttgtgtggaaaacgggttgcctaaggatgccgcatatatccagaatataatcatgactcggcttcaactattgctagatcattactattcaatgcatggcggcataaattcagaattagagcagccaactttaagtatctctgttcgaaacgcaacctacttaccatctcaactaggcggttacaatcatttgaatatgacccgactattctgccgcaatatcggcgacccgcttaccagttcttgggcggagtcaaaaagactaatggatgttggtctcctcagtcgtaagttcttagaggggatattatggagacccccgggaagtgggacgttttcaacactcatgcttgacccgttcgcacttaacattgattacctgaggccgccagagacaattatccgaaaacacacccaaaaagtcttgttgcaagattgcccaaatcccctattagcaggtgtcgttgacccgaactacaaccaagaattagagctgttagctcagttcttgcttgatcgggaaaccgttattcccagggctgcccatgccatcttcgagttatctgtcttgggaaggaaaaaacatatacaaggattggtagatactacaaagacaattattcagtgctcattggaaagacagccattgtcttggaggaaagttgagaacattgttacctacaacgcgcagtatttcctcggggccacccaacaggctgatactaatgtctcagaagggcagtgggtgatgccaggtaaccttaagaagcttgtgtccctcgacgattgctcggtcacgctgtctactgtatcacggcgcatatcatgggccaatctactgaactggagagctatagatggtctggaaaccccggatgtgatagagagtattgatggtcgccttgtacaatcatccaatcaatgtggcctatgtaatcaagggttgggatcctactcctggtttttcttgccctctgggtgtgtgttcgaccgtccacaagattctcgggtagttccaaagatgccatacgtggggtccaaaacagatgagagacagactgcatcagtgcaagctatacaaggatccacttgtcacctcagggcagcattgaggcttgtatcactctacctatgggcctatggagattctgacatatcatggctagaagctgcaacgctggctcaaacacggtgcaatgtctctctcgatgatttgcgaatcttgagccctcttccttcttcggcgaatttacaccacagattaaatgacggggtaacacaggttaaattcatgcccgccacatctagccgagtgtcaaagttcgtccaaatttgcaatgacaaccagaatcttatccgtgatgatgggagtgttgattccaatatgatttatcaacaggttatgatattagggcttggagagattgaatgcttgttagctgacccaattgatacaaacccagaacaattgattcttcatctacactctgataattcttgctgtctccgggagatgccaacgaccggctttgtacctgctctaggactaaccccatgtttaactgtcccaaagcataatccttacatttatgacgatagcccaatacccggtgatttggatcagaggctcattcagaccaaatttttcatggggtctgacaatttggataatcttgatatctaccagcagcgagctttactgagtaggtgtgtagcttatgatgtcatccaatcgatctttgcctgtgatgcaccagtctctcagaagaatgacgcaatccttcacactgattaccatgagaattggatctcagagttccgatggggtgaccctcgtattatccaagtaacggcaggctatgagttaattctgttccttgcataccagctttattatctcagagtgaggggtgaccgtgcaatcctgtgctatatcgacaggatactcaataggatggtatcttccaatctaggtagtctcatccagacactctctcatccagagattaggaggagattctcgttgagtgatcaagggtttcttgttgaaagagaactagagccaggtaagcccttggttaaacaagcggttatgttcttaagggactcggtccgctgcgctttagcaactatcaaggcaggaattgagcctgaaatctcccgaggtggttgtactcaggatgagctgagctttactcttaagcacttactatgtcggcgtctctgtgtaatcgctctcatgcattcagaagcaaagaacttggttaaagttagaaaccttcctgtagaagagaaaaccgccttattgtaccagatgttggtcactgaggccaatgctaggaaatcagggtctgccagcattatcataaacctagtctcggcaccccagtgggacattcatacaccagcattgtattttgtgtcaaagaaaatgctagggatgcttaagaggtcaaccacacccttggatataagtgacctctctgagaaccagaaccccgcacctgcagagcttagtgatgctcctggtcacatggcagaagaattcccctgtttgtttagtagttataacgctacatatgaagacacaatcacttacaatccaatgactgaaaaactcgccttgcatttggacaacagttccaccccatccagagcacttggtcgtcactacatcctgcggcctcttgggctttactcatccgcatggtaccggtctgcggcactactagcgtcaggggccctaaatgggttgcctgaggggtcgagcctgtatttaggagaagggtacgggaccaccatgactctgcttgagcccgttgtcaagtcttcaactgtttactaccatacattgtttgacccaacccggaacccttcacagcggaactataaaccagaaccacgggtattcacggattctatttggtacaaggatgatttcacacggccacccggtggtattatcaacctgtggggtgaagatatacgtcagagtgatatcacacagaaagacacggtcaacttcatactatctcagatcccgccaaaatcacttaagttgatacacgttgatattgagttctcaccagactccgatgtacggacactactatccggctattctcattgtgcactattggcctactggctattgcaacctggagggcgattcgcagttagggttttcttaagtgaccatatcatagttaacttggtcactgcgatcctgtctgcttttgactccaatttggtgtgcattgcgtcaggattgacacacaaggatgatggggcaggttatatttgcgcgaaaaagcttgcaaatgttgaggcttcaagaattgagtactacttgaggatggtccatggttgtgttgactcattaaagatccctcatcaattaggaatcattaaatgggccgagggtgaggtgtcccagcttaccagaaaggcggatgatgaaataaattggcggttaggtgatccagttaccagatcatttgatccagtttctgagctaataattgcacgaacaggggggtctgtattaatggaatacggggcttttactaacctcaggtgtgcgaacttggtagatacatacaaacttctggcttcaattgtagagaccaccctaatggaaataagggttgagcaagatcagttggaagatagttcgaggagacaaatccaagtaatccccgctttcaacacaagatctgggggaaggatccgtacactgattgagtgtgctcagctgcagattatagatgttatttgtgtaaacatagatcacctctttcctaaacaccgacatgttcttgtcacacaacttacctaccagtcggtgtgccttggggatttgattgaaggtccccaaattaagacgtatctaagggccagaaagtggatccaacgtcggggactcaatgagacagttaaccatatcatcactggacaagtgtcacggaataaagcaagggatttttttaagaggcgcctgaagttggttggcttttcactctgcggaggttggagctacctctcactttagctgttcaggttgctgatcatcatgaacaatcggagtcggaatcgtaaacagaaagtcacaaaattgtggataaacaatgattgcattagtatttaataaaaaatatgtcttttatttcgt

SEQ ID NO: 82 - DNA, 15048 bp, Artificial Sequence
  Avian paramyxovirus 4 strain APMV-4/duck/Delaware/549227/2010, complete genome (GenBank JX987283.1)
acgaaaaagaagaataaaaggcagaagccttttaaaaggaaccctgggctgtcgtaggtgtgggaaggttgtattccgagtgcgcctccgaggcatctactctacacctatcacaatggctggtgtcttttcccagtatgagaggtttgtggacaatcaatctcaggtgtcaaggaaggatcatcggtccttagcaggagggtgccttaaagtgaacatccctatgcttgtcactgcatccgaagaccccaccacgcgttggcaactagcatgcttatctctgaggctcttgatttccaattcatcaaccagtgctatccgccagggagcaatactgaccctcatgtcattgccatcgcaaaacatgagagcaacagcagctattgctgggtccacgaatgcggctgttatcaacactatggaagtcttaagtgtcaatgactggaccccatcttttgacccaagaagtggtctatctgaggaggacgctcaggtgttcagagacatggcaagagatctgcctcctcagttcacttctggatcaccctttacatcagcattggcggaggggtttactcccgaggacactcatgacctgatggaggcactgactagtgtactgatacagatctggattctggtggccaaggccatgaccaatattgatggatctggggaggctaacgaaagacgccttgcaaaatacatccaaaagggacagctcaatcgtcagtttgcaattggcaatcctgcccgtctgataatccaacagacaatcaaaagctcattaactgtccgcaggttcttggtctctgagctccgcgcatcacgtggtgcagtaaaggagggttccccttactatgcagccgttggggatatccacgcttacatcttcaatgcaggattgacaccattcttgaccaccctgagatatggcattggcaccaagtacgccgctgtcgcactcagtgtgtttgctgcagacattgcaaaattgaagagtctactcaccctgtatcaagacaaaggtgtagaagctggatacatggcactccttgaagatccagattccatgcactttgcacctggaaacttcccacacatgtattcctatgcgatgggagtggcctcctatcacgaccctagcatgcgccaataccagtatgccaggaggtttctcagtcgtcccttctacctgctaggaagagacatggctgctaagaacacaggaactctggatgagcagctggcgaaagaactgcaagtgtcagagagggaccgcgctgcactgtctgccgcgattcaatcagcaatggaggggggagagtcagatgacttcccattgtcaggatccatgccggccctctctgagagcacacaaccggtcacccccaggactcaacagtcccagctctctcctcctcaatcatcaaacatgtcccaatcggcgcctaggaccccggactatcaacccgactttgagctgtagactatatccacacaccgacaatagctccagaagacccccttcccccccatacaccccacccggtcatccacaaagacccagtccaacatcccagcactattcccttttaattaaaaactggccgacagggtggggaaggaggactgttagctgccaccaacggtgtgcagcaatggattttacagacattgacgctgtcaactcactgattgagtcatcatcggcaattatagactccatacagcatggagggctgcaaccagcaggcactgttggcttatctcaaattccaaaagggataaccagtgcactgaataaagcctgggaagctgaggcggcaactgccggcagtggagacacccaacacaaacccgatgacccagaggaccaccaggctagggacacggagtccctggaagacacaggcaacgacccggccacacaggggactaacattgttgagacaccccacccagaagtactgtcagcagccaaagctagactcaagagacccaaagcagggaaagacacccatggcaatccccccactcaacccgatcactttttaaaggggggcctcccgagtccacaaccgacagcaccgcggatgcaaagtccacccaaccatggaagctccagcaccgccgatccccgccaatcacaaactcaggatcattcccccaccggagagaaatggcaattgtcaccgacaaagcaaccggagacatcgaactggtggagtggtgcaacccagggtgtacagcagtccgaattgaaccagccagacttgactgtgtatgcggacactgccccaccatctgcagtctctgcatgtatgacgactgatcaggtacagttgttgatgaaggaggttgctgacataaaatcactcctccaggcactagtaaggaatctagctgtcttgccccaactaaggaatgaggttgcagcaatcagaacatcacaggccatgatagaggggacactcaattcaattaagattcttgatcctggaaattatcaggaatcatcactaaacagttggttcaaacctcgccaggaacacactgttattgtgtcaggaccagggaatccactggccatgccgactccagttcaggacagtaccatattcttagatgagctagcaagacctcatcctaatttggtcaatccgtctccgcccgtcaccagcaccaatgttgaccttggcccacagaagcaggctgcaatagcctacgtttccgccaagtgcaaggacccagggaaacgggaccagctttcaaggcttattgaacgggcggctaccttgagtgagatcaacaaggttaaaagacaggctctcgggctctaaattaatcaaccacccgttgcaacgatcgagacaacaataaaaatccccctgaatcacatgaccaaatctgcataccactcacatcatccgcctatacccctcaccataaataccaccttagccgatttatttaaaagaaatcattcatcacaacctggtaatcataaactagggtggggaaggtctcttgtctgcaggaaggctcctctgtctccaggcacgcacccgtcaacccaccaataacacaatggcggacatggacacgatatacatcaacttgatggcagatgatccaacccatcaaaaagaattgctgtcattccctctgattccagtgactggacctgatgggaagaaagtgctccaacaccagatccggacccaatccttgctcacctcagacaaacaaacggagaggttcatctttctcaacacttacgggttcatctatgacacaaccccggacaagacaactttttccacccctgagcatatcaatcagcctaagaggacaatggtgagtgctgcgatgatgactattggtctggttcctgctacaatacccctgaatgaattgacggccactgtgtttaaccttaaagtaagagtgaggaaaagtgcgaggtatcgagaagtggtttggtaccagtgcaaccccgtaccagctctgctcgcagccaccagatttggccgccaagggggtcttgagtcgagcaccggagtcagtgtaaaggcacctgagaagattgattgtgagaaagattatacttactacccttatttcctatctgtgtgctacatcgccacttccaacctctttaaggtaccgaagatggttgccaatgcaaccaacagtcaattgtatcacctaaccatgcaggtcacatttgcatttccgaaaaacattcccccagccaatcagaaactcctgacacaggtagatgaaggatttgagggtaccgtggattgccattttgggaacatgctaaaaaaggataggaaagggaacatgaggactttgtctcaagcagcagataaggtcagaagaatgaatatccttgtgggaatatttgacttgcacggacctacactattcctggaatatactgggaaattgacaaaagccctgttggggttcatgtccaccagccgaacagcaatcatccccatatcacaactcaatcctatgctgagtcaactcatgtggagcagtgacgcccagatagtaaagttacgggtggtcatcactacatctaaacgtggcccgtgtgggggcgagcaggaatatgtgctggatcctaaattcacagttaagaaagaaaaggctcgactcaatccattcaagaaggcagcctaataattaaacctacaagatcccaagaattaaacagctctatacaattcataggttgatagaaatgccactacacagctaatgattttccagaaaatcacttagaaaaccaaatccttattagggtggggaagtagttgattgggtgtctaaacaaaagtgcttctttgcaactccccaccccgaagcaatcacaatgagaccattaaacacgcttttgaccgtgattcttatcatactcatcagctatttggtgattgttcattctagtgatgcggttgagaggccaaggactgagggaattaggggcgacctcattccaggtgcgggtatcttcgtgactcaagtccgacaactgcaaatctatcagcagtcagggtaccacgaccttgtcataagattattaccccttttaccaacggaactcaatgattgccaaaaagaagtagtcacagaatacaataatacagtatcacaattgttgcagcctatcaaaaccaacttggataccctattagcagatggtaatacgagggaagcggatatacagccgcggtttattggagcaataatagccacaggtgccttggcggtagcaacagtggcagaagtaactgcagctcaggcactctcccagtccaaaacaaatgctcaaaatattctcaagctaagagatagtatccaggccaccaaccaagcggtctttgaaatttcacaagggcttgaggcaactgcaactgtgctatcgaaactacagacagagctcaatgagaatattatcccaagcctgaacaatttatcctgtgctgccatggggaatcgtcttggtgtatcactctcactctatttaactctaatgactaccctctttggggaccaaattacgaacccagtgctgacaccaatttcttacagcacactatcggcaatggcaggtggtcatattggcccagtgatgagtaaaatattagccggatcggtcacgagccagttgggggcagaacaattgattgctagtggcttaatacaatcacaggtggtaggctatgattcccagtatcaattattggtaatcagggttaaccttgttcggattcaggaagtccagaataccagggttgtatcattaagaacgctagctgtcaatagagatggtggactttatagagcccaagttccacctgaggtagtcgaacgatccggcattgcagagcggttttacgcagatgattgtgttctcaccacgaccgactatatttgctcatcaatcagatcctctcggcttaatccagaattagtcaagtgtctcagtggggcacttgattcatgtacattcgagagggagagtgccctgttatcaactcctttctttgtgtacaataaggctgtcgtagcaaattgcaaagcggcaacatgcagatgcaacaaaccaccgtcaattattgctcaatattctgcatcagctctagtaaccatcaccactgacacctgtgccgatctcgaaattgagggttaccgtttcaacatacagactgaatctaactcgtgggttgcacctaactttactgtctcaacctcacagatagtgtcagttgatccaatagacatatcctctgacatcgcaaaaatcaacaattcgattgaggccgcacgagagcagctagaactgagcaaccagatcctatcccggattaacccccgaatcgtgaatgacgaatcactgatagctattatcgtgacaattgttgtgcttagtctccttgtagtcggtcttatcattgttctcggcgtgatgtataaaaatctcaagaaggtccaacgagctcaggctgctatgatgatgcagcaaatgagttcatcgcagcctgtaaccacaaaactggggacacccttctaggtgaataaatgcatcacctctttccttgatgagcgagatgtcttaatcattgataattatgccgtaaggctggtagggaatgtgctgaatctctcctcttcctttttaattaaaaacggttgaactgagggggagaatgtgcatggtagggtggggaaggtgtctgattcctacctatcgggccaactgtaccagtagaagctaacaggaattctaatgcagagtgacatggagggcagtcgtgataacctcacagtggatgatgagttaaagacaacatggaggttagcttacagagttgtatctctcctattaatggtgagtgctttgataatttctatagtaatcttgacgagggataacagccaaagcataatcacggcaatcaaccagtcatatgatgcagactcaaagtggcaaacagggatagaggggaaaatcacctctatcatgactgatacgcttgatactaggaatgcagctctcctccacattccactccaacttaatacacttgaagcaaacctattatcagccctcggtggcaacacaggaatcggccccggggatctagagcattgccgttatccagttcatgattctgcttacctgcatggagtcaaccgattacttatcaatcaaacggctgattatacagcagagggtccactagatcatgtgaacttcataccggcaccagttacgaccactggatgcactaggataccatctttttccgtgtcctcatccatttggtgttatactcacaatgtgattgaaactggttttaatgatcactcaggcagcaatcagtatattagcatgggggtgattaagagggctggcaacggcttgccttatttctcaaccgttgtgagtaagtatctgaccgacggattgaataggaaaagttgttctgtggctgctgggtctgggcattgctatcttctctgcagcctagtatcagagcccgagcctgacgactatgtatcaccagaccccacaccgatgaggttaggggttctgacatgggatgggtcctatactgaacaggtggtgcctgaaaggatattcaaaaacatatggagtgcaaattaccctggggtgggatcaggtgctattgtgggaaataaggtgttgttcccattttacggaggagtgaggaatgggtcgacacctgaggttatgaataggggaaggtattactacattcaagatcctaatgattattgtcctgatccactgcaagaccaaatcttaagggcagaacaatcatattatcctacacggtttggtaggaggatggtgatgcagggtgtcttagcgtgcccagtgtccaacaactcaacaattgccagccaatgccagtcctactatttcaacaactcattagggttcattggggcggaatctaggatttattacctaaatgggaacctctacctttaccaaagaagctcgagctggtggccccacccccagatttatctgcttgaccccagaattgcaagcccgggcactcagaacatcgactcaggcattaatctcaagatgttgaatgttaccgttattacacgaccgtcatctggtttttgtaatagtcagtcaagatgccctaatgactgcttattcggggtctattcagacgtctggcctcttagcctaacctcagatagtatattcgcattcacgatgtatttacaagggaagacaacacgtattgacccggcgtgggcactgttctccaatcacgcaattgggcatgaagctcgtctattcaacaaggaggtcagtgctgcttactccactaccacttgcttttcggacaccatccaaaaccaggtgtattgcctgagtatacttgaagttagaagtgagcttttggggccattcaagatagtaccattcctctaccgtgtcctataggtgcctgctcgatcgagaactccaaataatcgtggaattagtacttaatcttccctatggatatctgccttaattactgtcctaggtctctggattagcgccctttaaaccagttttttgatttttaattaaaaatagaagattagacctggactcggggagggagaagaacctattagggtggggaaggattactttactccatgactcacaatcgcacacacctgacctcatttccactgagaaggaaccctcctcaaatttgatttgcaatgtccaatcaagcagctgagattatactccctacctttcacctagagtcacccttaatcgagaacaaatgcttctactatatgcaattacttggtcttatgttgccgcatgatcattggagatggagggcatttgtcaactttacagtggatcaagcacaccttagaaaccgtaatcctcgcttgatggcccacatcgaccacactaaggataaactaagggctcatggtgtcttaggtttccatcagacccaaacaggtgagagccgtttccgtgtcttgcttcacccggaaaccttaccatggctatcagcaatgggaggatgcataaaccaagtccccaaagcatggcggaacactctgaagtccatcgagcacagtgtgaagcaggaggcaacacaactacaatcgcttatgaaaaaaacctcattgaaattaacaggagtaccctacttattttccaactgtaatcccgggaaaaccacaacaggcactatgcctgtattaagcgagatggcatcagagctcctatcaaatcccatctcccaattccaatcaacatgggggtgtgctgcttcagggtggcaccatattgttagcatcatgaggcttcaacagtatcaaagaaggacaggtaaagaggagaaggcgatcactgaggttcattttggttcagacacctgtctcattaatgcagactacaccgttatcttttccttacagagccgtgtaataacagttttaccttttgacgttgtcctcatgatgcaagacctgctcgaatctcgacgaaatgtcctgttctgtgcccgctttatgtaccccagaagccaattgcatgagaggataagcatgatactagctctcggagatcaacttgggaaaaaggcaccccaagttctatatgactttgttgcaacccttgaatcatttgcatacgcagctgtccaacttcatgacaataaccctatctacggtgggactttctttgaattcaatatccaagaattagaatctatcttgtctcctgcgcttagcaaggaccaggtcaacttctacattagtcaggttgtctcagcatacagtaacctccccccatctgaatcggcagaattgctatgcctgttacgcctatggggtcaccctttactaaatagcctcgatgcagcaaagaaagtcagagaatcaatgtgtgccgggaaggttcttgactacaatgccattcgattagtcttgtctttttaccatacattattgatcaatggatatcggaagaaacacaagggacgctggccaaatgtgaatcaacattcactactcaacccaatagtgaggcagctttactttgatcaagaagagatcccacattctgtcgccctcgaacattacttagacatctcaatgatagaatttgagaaaacttttgaggttgaactatctgacagcctaagcatctttttgaaagacaagtcgattgccttggacaaacaagagtggtacagcggttttgtttcagaagtgaccccaaagcacttgcggatgtctcgtcatgaccgcaagtccaccaacaggctcctgctggcctttatcaactcccctgaattcgatgttaaagaagagctaaaatacttgactacaggtgagtatgctactgatccaaatttcaacgtttcttactcacttaaagagaaggaagtaaagaaagaaggacgaatctttgcaaaaatgtcacaaaagatgagagcgtgccaggttatttgtgaagagttgctagcacatcatgtagcccctttgtttaaagagaatggtgtcacacagtcggaactatctctgacaaaaaatctgctagctatcagtcagttgagttataactcaatggctgctaaggtgcggttgctgagaccaggggacaaattcactgccgcacactatatgaccacagacctgaaaaagtactgccttaattggcgtcaccagtcagtcaaactgtttgccagaagcctagatcgactgttcgggctagatcatgctttttcttggatacatgtccgcctcaccaacagcaccatgtatgtggctgatccattcaatccaccagactcagatgcatgcccaaacttagacgacaacaaaaacacgggaattttcatcataagtgcacgaggtgggatagaaggcctccaacaaaaactgtggaccggcatatcaatcgcaatcgcgcaagcagctgcagccctcgaaggcttgagaattgctgctactttgcagggggacaaccaggttctagcgatcacgaaggaatttgtaaccccagtcccggaaggtgtcctccatgagcaattatctgaggcgatgtcccgatataaaaagactttcacataccttaattacttaatggggcatcaactgaaagataaagagacaatccaatccagtgatttctttgtttactctaaaaggatattctttaatgggtccattctgagtcaatgtctcaaaaacttcagtaagctcaccactaatgccaccacccttgccgagaacactgtagccggctgcagtgacatctcatcatgcatcgctcgttgtgtagaaaacgggttgccaaaggatgctgcatacatccagaacatagtcatgactcgacttcaactgttgctagatcactactattccatgcatggtggcataaactcagaattagaacagccgaccctaagtatttctgttcggaatgcaacctatttaccatctcagttgggcggttacaatcatctaaatatgacccgactattttgccgcaacatcggtgacccgctcactagttcctgggcagaagcaaagagactaatggaagttggcctgctcaatcgtaaattcctggagggaatattgtggcgacctccgggaagtgggacattctcaacacttatgcttgacccgtttgcgctgaacattgattacctcagaccaccagagacaataatccgaaagcatacccagaaggtcttgctgcaagattgccctaatcccctattagccggtgtggttgatccgaactacaaccaggaactggaactattagcgcagttcttgctcgaccgagagaccgttattcccagggcagctcatgctatctttgagctgtctgtcttggggaggaaaaaacatatacaagggttggtggacactacaaaaacgattatccagtgttcgctggaaagacaaccattgtcctggaggaaagttgagaacattatcacctataatgcgcagtatttccttggagccactcagcagattgatacagattcccctgaaaagcagtgggtgatgccaagcaacttcaagaagctcgtgtctcttgacgattgttcagtcacattgtctactgtttcccggcgtatatcttgggccaacctacttaattggagggcaatagatggcttggaaaccccagatgtgatagaaagtattgatgggcgccttgtgcaatcatccaatcagtgtggcctatgtaatcaaggattaagttcctactcctggttcttcctcccctccggatgtgtgtttgatcgtccacaagactccagggtagtaccgaaaatgccgtatgtgggatccaagacagatgagaggcagactgcgtcggtacaagctatacagggatccacatgtcaccttagagcagcattgagacttgtatcactctacctttgggcttatggggattctgatatatcatggctggaagccgcgacactagcccaaacacggtgcaatatttcccttgatgatctgcgaatcctgagccctctaccttcctcggcaaatttacaccacagattaaatgacggggtaacacaagtgaaattcatgcctgctacatcaagccgagtatcaaagtttgtccagatttgcaatgacaaccagaatcttatccgtgatgatgggagtgtggattccaatatgatttatcagcaagtcatgatattaggacttggggaatttgagtgcttgttggccgacccaatcgatactaacccagagcaattgattcttcatctacactctgacaattcttgctgcctccgggagatgccaacaaccggctttgtgcctgctttgggattaaccccatgcttaactgtaccaaagcaaaatccatatatttatgacgagagtccaatacctggtgacctggatcaacggctcatccaaacaaagtttttcatgggttctgataatctagacaaccttgatatctatcagcaacgagcgttactaagtcggtgtgtggcttatgatgttatccaatcagtatttgcttgtgatgcaccagtttctcagaagaatgatgcaatcctccatactgactatcatgagaattggatctcagagttccgatggggtgaccctcggataattcaagtgacagcaggttatgaattgatcttgtttcttgcttaccagctttattaccttagagtgaggggtgaccgtgcaatcctgtgctatattgataggatactgaataggatggtgtcatcaaatctaggcagccttatccagacactctcccatccggagattaggaggaggttttcattaagtgatcaaggattccttgttgaaagggaactagagccaggcaaacctttggtaaaacaagcagtcatgttcctaagggactcagtccgatgtgctttagcaactatcaaggcaggagtcgagccggagatctcccgaggtggctgtacccaagatgagttgagtttcaccctcaagcacttgctatgtcgacgtctctgtataattgctctcatgcattcagaagcaaagaacttggtcaaggtcagaaatctcccagtagaggaaaaatctgctttactataccagatgttggtcaccgaagctaatgcccggaaatcaggatctgctagcatcatcataggcttaatttcggcacctcagtgggatatccataccccagcactgtactttgtatcaaagaagatgctaggaatgctcaaaaggtcaactacaccattggatgtaaatgatctgtctgagagccaggaccttatgccaacagagttgagtgatggtcctggtcacatggcagagggatttccctgtctatttagtagttttaacgctacatatgaagacacaattgtttataatccgatgactgaaaagcctgcagtacatttggacaatggatccaccccatccagggcgctaggtcgccactacatcttgcggcccctcgggctttactcgtctgcatggtaccggtctgcagcactcttagcatcaggtgctctcaatgggttaccggagggatcaagcctatacttgggagaagggtatgggaccaccatgactctgctcgaacccgtcgtcaagtcctcaactgtttattaccacacattgtttgacccgacccggaatccctcacagcggaattacaaaccagagccgcgagtcttcactgattccatctggtacaaggatgacttcacacgaccgcctggtggcattgtaaatctatggggtgaagatgtgcgtcagagtgacgtcacacagaaagacacagttaatttcatattatcccggatcccacccaaatcactcaaactgatccatgttgacattgaattctcaccagactccaatgtacggacactactatctggttactcccattgcgcattattggcctactggctattgcaacctggagggcgatttgcggttagggtcttcctgagtgaccatctcttagtaaacttggtcactgctattctgtctgctttcgactctaatctactgtgtattgcatctggattgacacacaaagatgatggggcaggttacatttgtgctaagaagcttgccaatgttgaggcatcaaggattgagcactacttaaggatggtccatggttgcgttgattcattaaagatcccccaccaactagggatcattaagtgggctgaaggtgaggtgtctcggctcacaaaaaaggcagatgaagaaataaattggcgattaggtgacccggttactagatcatttgatccagtttccgagttaataatcgcacggacaggggggtctgtattaatggaatatgggactttcattaatctcaggtgttcaaacctggcagatacatataaacttttggcttcaatcgtggagaccaccttgatggagataagggttgaacaagatcaattggaagacaactcaagaagacaaattcaggtggtccccgcctttaatacgagatccggggggaggatccgtacattgattgagtgtgcccagctgcaggttatagatgtcatatgtgtaaacatagatcacctcttccccaaacatcgacatgttcttgttacacaactcacttaccagtcagtgtgccttggagacttgatcgaggggccccaaattaagatgtatctaagggccaggaagtggatccaacgtagaggactcaatgagacaattaaccatatcatcactggacagatatcacgaaataaggcaagggatttcttcaagaggcgcctgaagttggttggcttctcgctttgcggcggttggagttacctctcactttagttacttaggttgttgatcattgtgaaaaatcggagtcggaatcgcaaataaaaacatacaaaattgcaaatttacaataatcgcattaatatttaataaaaaatatgtcttttatttcgt

SEQ ID NO: 83 - DNA, 15185 bp, Artificial Sequence
  Newcastle disease virus strain LaSota, complete genome with modification in 5408-5409-5410 nucleotides resulting in L289A substitution
accaaacagagaatccgtgagttacgataaaaggcgaaagagcaattgaagtcacacgggtagaaggtgtgaatctcgagtgcgagcccgaagcacaaactcgagaaagccttctgccaacatgtcctccgtatttgatgagtacgaacagctcctcgcggctcagactcgccccaacggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaataataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccattactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatcggatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggtcgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcagacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatccctgctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggtgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacccgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactgccccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgccctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaaaactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaacccgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttccacgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggtaaataatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagtcgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaagacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagctcaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctagacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagaccacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagtgacctgacctcagataaagcagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcaaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaaattgacactgcgctgattgaagccgggggacagcccgtccgtccattctgtgcggagagtctggtgagcacgctagcgaacataactcagataacccagatcatcgctagccacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt

SEQ ID NO: 84 - DNA, 15186 bp, Artificial Sequence
  Sequence NDV LaSota L289A genome
accaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactgccccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt

SEQ ID NO: 85 - DNA, 18632 bp, Artificial Sequence
  Plasmid pNDV-LaSotaL289A
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctttaatacgactcactatagggaccaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactgccccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtgcgcgcggccggcatggtcccagcctcctcgctggcgccggctgggcaacattccgaggggaccgtcccctcggtaatggcgaatgggacgtcgactgctaacaaagcccgaaaggaagctgagttggctgctgccaccgctgagcaataactagcataaccccttggggcctctaaacgggtcttgaggggttttttgctgaaaggaggaactatatgcgcgcagatctgtcatgatgatcattgcaattggatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 86 - DNA, 15054 bp, Artificial Sequence
  Full length genome of APMV4 from RNA sequencing and confirmation of the 5′ and 3′ ends by RACE
acgaaaaagaagaataaaaggcagaagccttttaaaaggaaccctgggctgtcgtaggtgtgggaaggttgtattccgagtgcgcctccgaggcatctactctacacctatcacaatggctggtgtcttctcccagtatgagaggtttgtggacaatcaatcccaagtgtcaaggaaggatcatcggtccttagcaggaggatgccttaaagttaacatccctatgcttgtcactgcatctgaagaccccaccactcgttggcaactagcatgcttatctctaaggctcctgatctccaactcatcaaccagtgctatccgtcagggggcaatactgactctcatgtcattaccatcacaaaacatgagagcaacagcagctattgctggttccacaaatgcagctgttatcaacaccatggaagtcttaagtgtcaacgactggaccccatccttcgaccctaggagcggtctttctgaggaagatgctcaagttttcagagacatggcaagagatctgccccctcagttcacctctggatcacccttcacatcagcattggcggaggggttcactcctgaagatactcatgacctgatggaggccttgaccagtgtgctgatacagatctggatcctggtggctaaggccatgaccaacattgacggctctggggaggccaatgaaagacgtcttgcaaagtacatccaaaaaggacagcttaatcgtcagtttgcaattggtaatcctgcccgtctgataatccaacagacaatcaaaagctccttaactgtccgtaggttcttggtctctgagcttcgtgcgtcacgaggtgcagtaaaagaaggatccccttactatgcagctgttggggatatccacgcttacatctttaatgcgggattgacaccattcttgaccaccttaagatacgggataggcaccaagtacgccgctgttgcactcagtgtgttcgctgcagatattgcaaagttgaagagcctacttaccctgtaccaggacaagggtgtagaagctggatacatggcactccttgaggatccagactccatgcactttgcacctggaaacttcccacacatgtactcctatgcaatgggggtagcttcttaccatgatcctagcatgcgccaataccaatacgccaggaggttcctcagccgtcctttctacttactaggaagggacatggccgccaagaacacaggcacgctggatgagcaactggcgaaggaactgcaagtatcagagagagatcgcgccgcattatccgctgcgattcaatcagcgatggaggggggagagtccgacgacttcccactgtcgggatccatgccggctctctctgagaatgcgcaaccagttacccccagacctcaacagtcccagctctctcccccccaatcatcaaacatgccccaatcagcacccaggaccccagactatcaacccgactttgaactgtaggcttcatcaccgcaccaacaacagcccaagaagaccacccctccccccacacatctcacccagccacccataaagactcagtcccacgccccagcatctccttcatttaattaaaaaccgaccaacagggtggggaaggagagtcattggctactgccaattgtgtgcagcaatggattttactgacattgatgctgtcaactcattgatcgaatcatcatcggcaatcatagactccatacagcatggagggctgcaaccagcgggcaccgtcggcctatcgcagatcccaaaagggataaccagcgcattaaccaaggcctgggaggctgaggcggcaactgccggtaatggggacacccaacacaaatctgacagtccggaggatcatcaggccaacgacacagattcccctgaagacacaggtactgaccagaccacccaggaggccaacatcgttgagacaccccaccccgaggtgctgtcagcagccaaagccagactcaagaggcccaaagcagggagggacacccgcgacaactcccctgcgcaacccgatcatcttttaaaggggggcctcctgagcccacaaccagcagcatcatgggtgcaaaatccacccagtcatggaggtcccggcaccgccgatccccgcccatcacaaactcaggatcattcccccaccggagagaaatggcgattgtcaccgacaaagcaaccggagacattgaactggtggagtggtgcaacccggggtgcacagcagtccgaattgaacccaccagactcgactgtgtatgcggacactgccccaccatctgtagcctctgcatgtatgacgactgatcaggtacaactactaatgaaggaggttgctgacataaaatcactccttcaggcgttagtgaggaacctcgctgtcttgccccaattgaggaatgaggttgcagcaatcagaacatcacaggccatgatagaggggacactcaattcgatcaagattcttgaccctgggaattatcaggaatcatcactaaacagttggttcaaacctcgccaagatcacactgttgttgtgtctggaccagggaatccattggccatgccaaccccagtccaagacaacaccatattcctggacgagctagccagacctcatcctagtgtggtcaatccatccccgcccatcaccaacaccaatgttgaccttggcccacagaagcaggctgcaatagcctatatctccgctaaatgcaaggatccggggaaacgagatcagctatcaaggctcattgagcgagcaaccaccccaagtgagatcaacaaagttaaaagacaagcccttgggctctagatcactcgatcacccctcatggtgatcacaacaataatcagaacccttccgaaccacatgaccaacccagcccaccgcccacaccgtccatcgacatcccttgccaaacatcctgccgtagctgatttattcaaaagagctcatttgatatgacctggtaatcataaaatagggtggggaaggtgctttgcctgtaagggggctccctcatcttcagacacgtgcccgccatctcaccaacagtgcaatggcagacatggacacggtgtatatcaatctgatggcagatgacccaacccaccaaaaagaactgctgtcctttcctctcatccctgtgaccggtcctgacgggaagaaggaactccaacaccagatccggacccaatccttgctcgcctcagacaaacaaactgaacggttcatcttcctcaacacttacggattcatctatgacaccacaccggacaagacaactttttccaccccagagcatattaatcagcctaagaggacgacggtgagtgccgcgatgatgaccattggcctggttcccgccaatatacccctgaacgaactaacggctactgtgttcagccttaaagtaagagtgaggaaaagtgcgaggtatcgggaagtggtctggtatcaatgcaatccagtaccggccctgcttgcagccaccaggtttggtcgccaaggaggtctcgagtcgagcactggagtcagtgtaaaggctcccgagaagatagattgtgagaaggattatacctactacccttatttcttatctgtgtgctacatcgccacctccaacctgttcaaggtaccgaggatggttgctaatgcaaccaacagtcaattataccaccttaccatgcaggtcacatttgcctttccaaaaaacatccctccagccaaccagaaactcctgacacaggtggatgagggattcgagggcactgtggattgccattttgggaacatgctgaaaaaggatcggaaagggaacatgaggacactgtcccaggcggcagataaggtcagacgaatgaatattcttgttggtatctttgacttgcatgggccaacgctcttcctggagtataccgggaaactgacaaaggctctgctagggttcatgtccaccagccgaacagcaatcatccccatatctcagctcaatcccatgctgagtcaactcatgtggagcagtgatgcccagatagtaaagttaagggttgtcataactacatccaaacgcggcccgtgcgggggtgagcaggagtatgtgctggatcccaaattcacagttaagaaagaaaaggctcgactcaaccctttcaagaaggcagcctaatgatttaatccgcaagatcccagaaatcagaccactctatactatccactgatcactggaaatgtaattgtacagttgatgaatctgtgaagaatcaattaaaaaaccggatccttattagggtggggaagtagttgattgggtgtctaaacaaaagcatttcttcacacctccccgccacgaaacaaccacaatgaggctatcaaacacaatcttgaccttgattctcatcatacttaccggctatttgataggtgtccactccaccgatgtgaatgagaaaccaaagtccgaagggattaggggtgatcttacaccaggtgcgggtattttcgtaactcaagtccgacagctccagatctaccaacagtctgggtaccatgatcttgtcatcagattgttacctcttctaccaacagagcttaatgattgtcaaagggaagttgtcacagagtacaataacactgtatcacagctgttgcagcctatcaaaaccaacctggatactttgttggcagatggtagcacaagggatgttgatatacagccgcgattcattggggcaataatagccacaggtgccctggctgtagcaacggtagctgaggtaactgcagctcaagcactatctcagtcaaaaacgaatgctcaaaatattctcaagttgagagatagtattcaggccaccaaccaagcagtttttgaaatttcacagggactcgaagcaactgcaaccgtgctatcaaaactgcaaactgagctcaatgagaatatcatcccaagtctgaacaacttgtcctgtgctgccatggggaatcgccttggtgtatcactctcactctatttgaccttaatgaccactctatttggggaccagatcacaaacccagtgctgacgccaatctcttacagcaccctatcggcaatggcgggtggtcacattggtccagtgatgagtaagatattagccggatctgtcacaagtcagttgggggcagaacaactgattgctagtggcttaatacagtcacaggtagtaggttatgattcccagtatcagctgttggttatcagggtcaaccttgtacggattcaggaagtccagaatactagggttgtatcactaagaacactagcagtcaatagggatggtggactttacagagcccaggtgccacccgaggtagttgagcgatctggcattgcagagcggttttatgcagatgattgtgttctaactacaactgattacatctgctcatcgatccgatcttctcggcttaatccagagttagtcaagtgtctcagtggggcacttgattcatgcacatttgagagggaaagtgcattactgtcaactcccttctttgtatacaacaaggcagtcgtcgcaaattgtaaagcagcgacatgtagatgtaataaaccgccatctatcattgcccaatactctgcatcagctctagtaaccatcaccaccgacacttgtgctgaccttgaaattgagggttatcgtttcaacatacagactgaatccaactcatgggttgcaccaaacttcacggtctcaacctcacaaatagtatcggttgatccaatagacatatcctctgacattgccaaaattaacaattctatcgaggctgcgcgagagcagctggaactgagcaaccagatcctttcccgaatcaacccacggattgtgaacgacgaatcactaatagctattatcgtgacaattgttgtgcttagtctccttgtaattggtcttattattgttctcggtgtgatgtacaagaatcttaagaaagtccaacgagctcaagctgctatgatgatgcagcaaatgagctcatcacagcctgtgaccaccaaattggggacacccttctaggtgaataatcatatcaatccattcaataatgagcgggacataccaatcaccaacgactgtgtcacaaggccggttaggaatgcaccggatctctctccttcctttttaattaaaaacggttgaactgagggtgagggggggggtgtgcatggtagggtggggaaggtagccaattcctgcccattgggccgaccgtaccaagagaagtcaacagaagtatagatgcagggcgacatggagggtagccgtgataacctcacagtagatgatgaattaaagacaacatggaggttagcttatagagttgtatccctcctattgatggtgagtgccttgataatctctatagtaatcctgacgagagataacagccaaagcataatcacggcgatcaaccagtcgtatgacgcagactcaaagtggcaaacagggatagaagggaaaatcacctcaatcatgactgatacgctcgataccaggaatgcagctcttctccacattccactccagctcaatacacttgaggcaaacctgttgtccgccctcggaggtaacacgggaattggccccggagatctagagcactgtcgttatccggttcatgactccgcttacctgcatggagtcaatcgattactcatcaatcaaacagctgactacacagcagaaggccccctggatcatgtgaacttcattccggcaccagttacgactactggatgcacaaggatcccatccttttctgtatcatcatccatttggtgctatacacacaatgtgattgaaacaggttgcaatgaccactcaggtagtaatcaatatatcagtatgggggtgattaagagggctggcaacggcttaccttacttctcaacagtcgtgagtaagtatctgaccgatgggttgaatagaaaaagctgttccgtagctgccggatccgggcattgttacctcctttgtagcctagtgtcagagcccgaacctgatgactatgtgtcaccagatcccacaccgatgaggttaggggtgctaacaagggatgggtcttacactgaacaggtggtacccgaaagaatatttaagaacatatggagcgcaaactaccctggggtagggtcaggtgctatagtaggaaataaggtgttattcccattttacggcggagtgaagaatggatcaacccctgaggtgatgaataggggaagatattactacatccaggatccaaatgactattgccctgacccgctgcaagatcagatcttaagggcagaacaatcgtattatcctactcgatttggtaggaggatggtaatgcagggagtcctaacatgtccagtatccaacaattcaacaatagccagccaatgccaatcttactatttcaacaactcattaggattcatcggggcggaatctaggatctattacctcaatggtaacatttacctttatcaaagaagctcgagctggtggcctcacccccaaatttacctacttgattccaggattgcaagtccgggtacgcagaacattgactcaggcgttaacctcaagatgttaaatgttactgtcattacacgaccatcatctggcttttgtaatagtcagtcaagatgccctaatgactgcttattcggggtttattcagatgtctggcctcttagccttacctcagacagcatatttgcatttacaatgtacttacaagggaagacgacacgtattgacccagcttgggcgctattctccaatcatgtaattgggcatgaggctcgtttgttcaacaaggaggttagtgctgcttattctaccaccacttgtttttcggacaccatccaaaaccaggtgtattgtctgagtatacttgaagtcagaagtgagctcttgggggcattcaagatagtgccattcctctatcgtgtcttataggcacctgcttggtcaagaaccctgagcagccataaaattaacacttgatcttccttaaaaacacctatctaaattactgtctgagatccctgattagttaccctttcaatcaatcaattaatttttaattaaaaacggaaaaatgggcctagttccaaggaaaggatgggacccattagggtggggaaggattactttgttccttgactcgcacccacgtacacccaatcccattcctgtcaagaaggaacccttcccaaactcaccttgcaatgtccaatcaggcagctgagattatactacccaccttccatctggaatcacccttgatcgagaataagtgcttctactacatgcaattacttggtctcgtgttaccacatgatcactggagatggagggcattcgtcaattttacagtggatcaagcacaccttaaaaatcgtaatccccgcttaatggcccacatcgatcacactaaggatagactaagggctcatggtgtcttgggtttccaccagactcagacaagtgagagccgtttccgtgtcttgctccatcctgaaactttaccttggctatcagcaatgggaggatgcatcaaccaggttcccaaggcatggcggaacactctgaaatctatcgagcacagtgtgaagcaggaggcgactcaactgaagttactcatggaaaaaacctcactaaagctaacaggagtatcttacttattctccaattgcaatcccgggaaaactgcagcgggaactatgcccgtactaagtgagatggcatcagaactcttgtcaaatcccatctcccaattccaatcaacatgggggtgtgctgcttcagggtggcaccatgtagtcagcatcatgaggctccaacagtatcaaagaaggacaggtaaggaagagaaagcaatcactgaagttcagtatggctcggacacctgtctcattaatgcagactacaccgtcgttttttccgcacaggaccgtgtcatagcagtcttgcctttcgatgttgtcctcatgatgcaagacctgcttgaatcccgacggaatgtcttgttctgtgcccgctttatgtatcccagaagccaactacatgagaggataagtacaatactggcccttggagaccaactcgggagaaaagcaccccaagtcctgtatgatttcgtagctaccctcgaatcatttgcatacgctgctgtccaacttcatgacaacaaccctatctacggtggggctttctttgagttcaatatccaagaactggaagctattttgtcccctgcacttaataaggatcaagtcaacttctacataagtcaagttgtctcagcatacagtaaccttcccccatctgaatcagcagaattgctatgcttactacgcctgtggggtcatcccttgctaaacagtcttgatgcagcaaagaaagtcagagaatctatgtgtgctgggaaggttcttgattataatgctattcgactagttttgtctttttatcatacgttattaatcaatgggtatcggaagaaacataagggtcgctggccaaatgtgaatcaacattcactactcaacccgatagtgaagcagctttactttgatcaggaggagatcccacactctgttgcccttgagcactatttagatatctcgatgatagaatttgagaagacttttgaagtggaactatctgatagtctaagcatctttctgaaggataagtcgatagctttggataaacaagaatggcacagtggttttgtctcagaagtgactccaaagcacctacgaatgtctcgtcatgatcgcaagtctaccaataggctattgttagcctttattaactcccctgaattcgatgttaaggaagagcttaaatatttgactacaggtgagtatgccactgacccaaatttcaatgtctcttactcactgaaagagaaggaagttaagaaagaagggcgcattttcgcaaagatgtcacagaaaatgagagcatgccaggttatttgtgaagagttactagcacatcatgtggctcctttgtttaaagagaatggtgttacacaatcggagctatccctgacaaagaatttgttggctattagccaactgagttacaactcgatggccgctaaggtgcgattgctgaggccaggggacaagttcaccgctgcacactatatgaccacagacctaaaaaagtactgccttaactggcggcaccagtcagtcaaattgttcgccagaagcctggatcgactatttgggttagaccatgctttttcttggatacacgtccgtctcaccaatagcactatgtacgttgctgacccattcaatccaccagactcagatgcatgcacaaatttagacgacaataagaacactgggatttttattataagtgctcgaggtggtatagaaggccttcaacagaaactatggactggcatatcaattgcaatcgcccaggcggcagcagccctcgagggcttacgaattgctgccactttgcagggggataaccaggttttagcgattacgaaagaattcatgaccccagtctcggaggatgtaatccacgagcagctatctgaagcgatgtcgcgatacaagaggactttcacataccttaattatttaatggggcaccaattgaaggataaagaaaccatccaatccagtgacttcttcgtttactccaaaaggatcttcttcaatgggtcaatcctaagtcaatgcctcaagaacttcagtaaactcactaccaatgccactacccttgctgagaacactgtagccggctgcagtgacatctcctcatgcatagcccgttgtgtggaaaacgggttgcctaaggatgctgcatatgttcagaatataatcatgactcggcttcaactgttgctagatcactactattctatgcatggtggcataaactcagagttagagcagccaactctaagtatccctgtccgaaacgcaacctatttaccatctcaattaggcggttacaatcatttgaatatgacccgactattctgtcgcaatatcggtgacccgcttactagttcttgggcagagtcaaaaagactaatggatgttggccttctcagtcgtaagttcttagaggggatattatggagacccccgggaagtgggacattttcaacactcatgcttgatccgttcgcacttaacattgattacttaaggccaccagagacaataatccgaaaacacacccaaaaagtcttgttgcaggattgtcctaatcctctattagcaggtgtagttgacccgaactacaaccaggaattagaattattagctcagttcctgcttgatcgggaaaccgttattcccagggctgcccatgccatctttgaactgtctgtcttgggaaggaaaaaacatatacaaggattggttgatactacaaaaacaattattcagtgctcattagaaagacagccactgtcctggaggaaagttgagaacattgtaacctacaatgcgcagtatttcctcggggccacccagcaggttgacaccaatatctcagaaaggcagtgggtgatgccaggtaatttcaagaagcttgtatctcttgacgattgctcagtcacgttgtccactgtgtcacggcgcatttcttgggccaatctacttaactggagggctatagatggtttggaaactccagatgtgatagagagtattgatggccgccttgtgcaatcatccaatcaatgcggcctatgtaatcaaggattgggctcctactcctggttcttcttgccctccgggtgtgtgttcgaccgtccacaagattctcgagtggttccaaagatgccatacgtgggatccaaaacggatgagagacagactgcgtcagtgcaggctatacagggatccacatgtcaccttagagcagcattgagacttgtatcactctacctttgggcctatggagattctgacatatcatggctagaagccgcgacattggctcaaacacggtgcaatatttctcttgatgacctgcggatcctgagccctcttccttcctcggcaaatttacaccacagattgaatgacggggtaacacaagtgaaattcatgcccgccacatcgagccgggtgtcaaagttcgtccaaatttgcaatgacaaccagaatcttatccgtgatgatgggagtgttgattccaatatgatttatcagcaggttatgatattagggcttggagagattgaatgtttgttagctgacccaatcgatacaaacccagaacaactgattcttcacctacactctgataattcttgctgtctccgggagatgccaacgaccggttttgtacctgctttaggattgaccccatgcttaactgtcccaaagcacaatccgtatatttatgatgatagcccaatacccggtgatttggatcagaggctcattcaaaccaaattctttatgggttctgacaatctagataatcttgatatctaccagcagcgagctttactgagtcggtgtgtggcttatgacattatccaatcagtattcgcttgcgatgcaccagtatctcagaagaatgatgcaatccttcacactgactaccatgaaaattggatctcagagttccgatggggtgaccctcgcataatccaagtaacagcaggttacgagttaattctgttccttgcataccagctttattatctcagagtgaggggtgaccgtgcaatcctgtgttatattgataggatactcaacaggatggtatcttccaatctaggcagtctcatccagacgctctctcatccggagattaggaggagattttcattgagtgatcaagggttccttgtcgaaagggagctagagccaggtaagccactggtaaaacaagcggttatgttcctaagggactcagtccgctgcgctttagcaactatcaaggcaggaattgagcctgagatctcccgaggtggctgtacccaggatgagctgagctttacccttaagcacttactatgtcggcgtctctgtataattgctctcatgcattcggaagcaaagaacttggtcaaagttagaaaccttccagtagaggaaaaaaccgccttactataccagatgttgatcactgaggccaatgccaggagatcagggtctgctagtatcatcataagcttagtttcagcaccccagtgggacattcatacaccagcgttgtattttgtatcaaagaaaatgctggggatgctcaaaaggtcaaccacacccttggatataagtgacctttctgagagccagaacctcacaccaacagaattgaatgatgttcctggtcacatggcagaggaatttccctgtttgtttagcagttataacgctacatatgaagacacaattacttacaatccaatgactgaaaaactcgcagtgcacttggacaatggttccaccccttccagagcgcttggtcgtcactacatcctgcgaccccttgggctttactcgtctgcatggtaccggtctgcagcactattagcgtcaggggccctcagtgggttgcctgaggggtcaagcctgtacttgggagaggggtatgggaccaccatgactctacttgagcccgttgtcaagtcctcaactgtttactaccatacattgtttgacccaacccggaatccttcacagcggaactacaaaccagaaccgcgggtattcactgattccatttggtacaaggatgatttcacacgaccacctggtggcattgtaaatctatggggtgaagacgtacgtcagagtgatattacacagaaagacacggttaatttcatattatctcgggtcccgccaaaatcactcaaattgatacacgttgatattgagttctccccagactctgatgtacggacgctactatctggctattcccattgtgcactattggcctactggctactgcaacctggagggcgatttgcggttagagttttcttaagtgaccatatcatagtcaacttggtcactgccattctgtccgcttttgactctaatctggtgtgcattgcgtcaggattgacacacaaggatgatggggcaggttatatttgtgcaaagaagcttgcaaatgttgaggcttcaagaattgagtattacttgaggatggtccacggctgtgttgactcattaaaaattcctcatcaattaggaatcattaaatgggctgagggtgaagtgtcccgacttaccaaaaaggcggatgatgaaataaactggcggttaggtgatccagttaccagatcatttgatccggtttctgagctaataattgcgcgaacagggggatcagtattaatggaatacgggacttttactaacctcaggtgtgcgaacttggcagatacatataaacttttggcttcaattgtagagaccaccttaatggaaataagggttgagcaagatcagttggaagatgattcgaggagacaaatccaggtagtccctgcttttaatacaagatccgggggaaggatccgtacattgattgagtgtgctcagctgcaggtcatagatgttatctgtgtgaacatagatcacctctttcccaaacaccgacatgctcttgtcacacaacttacttaccagtcagtgtgccttggggacttgattgaaggcccccaaattaagacatatctaagggccaggaagtggatccaacgtaggggactcaatgagacaattaaccatatcatcactggacaagtgtcgcggaataaggcaagggattttttcaagaggcgcctgaagttggttggcttttcgctctgtggcggttggggctacctctcactttagctgcttagattgttgattattatgaataatcggagtcgaaatcgtaaatagaaagacataaaattgcaaataagcaatgatcgtattaatatttaataaaaaatatgtcttttatttctt

SEQ ID NO: 87 - DNA, 1290 bp, Artificial Sequence
  Additional transcription unit encoding mVEGF-C
ccgcggttagaaaaaatacgggtagaaccgccaccatgcatctgctgtgtttcctgtcgctcgcctgctcactgctggcggcggcacttatcccgtccccacgggaggctcctgccaccgtggccgccttcgaatctgggctgggcttcagcgaagccgagcccgatggcggagaggtcaaggcattcgaaggaaaggacctcgaagaacagctgagatccgtgtcctccgtggacgaactcatgtccgtcctgtaccccgattactggaagatgtacaaatgccagctccggaagggcggttggcagcagcccactctgaacactcgcacgggagattccgtgaagtttgccgccgcccactacaatactgagattctcaagtccatcgacaacgaatggaggaaaacccagtgtatgccgcgcgaagtctgcattgacgtgggaaaggagttcggcgctgccaccaacaccttctttaagcctccctgcgtgtcggtgtatcgctgcgggggatgctgcaacagcgaaggccttcagtgcatgaacaccagcaccggatacctcagcaagactctcttcgaaatcactgtgccgctgtcacaaggcccgaagcctgtgaccatttccttcgccaaccacacctcctgtcggtgcatgagcaagctggatgtgtacagacaggtgcactccatcatccggagatcgttgcctgccaccctgccgcagtgccaagcggctaacaagacctgtcccaccaactacgtgtggaacaactatatgtgtcggtgcctggcacagcaggactttatcttctactccaacgtggaggacgactcgactaacggtttccacgacgtgtgcggacccaacaaggagctggatgaggatacttgtcagtgcgtgtgcaagggtggcctgcgcccgtcctcctgcggaccacataaggaactggacagggactcgtgccaatgcgtctgcaagaacaagctgttccctaactcctgcggggcgaaccgcgaattcgacgagaacacctgtcagtgtgtgtgcaagcggacttgcccgaggaatcagcctcttaaccccggaaaatgcgcctgcgaatgcacagagaacacccagaagtgcttcttgaaagggaagaagttccaccaccaaacctgttcatgctaccggcgcccatgtgccaaccggctgaagcactgcgacccgggattgagcttcagcgaggaggtctgcagatgcgtgccgtcatactggaagcgacctcatctgaattagcccgcgg

SEQ ID NO: 88 - DNA, 19916 bp, Artificial Sequence
  Rescue plasmid pNDV-mVEGF-C with the mVEGF-C transcription unit inserted at the unique Sac II site
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctttaatacgactcactatagggaccaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggttagaaaaaatacgggtagaaccgccaccatgcatctgctgtgtttcctgtcgctcgcctgctcactgctggcggcggcacttatcccgtccccacgggaggctcctgccaccgtggccgccttcgaatctgggctgggcttcagcgaagccgagcccgatggcggagaggtcaaggcattcgaaggaaaggacctcgaagaacagctgagatccgtgtcctccgtggacgaactcatgtccgtcctgtaccccgattactggaagatgtacaaatgccagctccggaagggcggttggcagcagcccactctgaacactcgcacgggagattccgtgaagtttgccgccgcccactacaatactgagattctcaagtccatcgacaacgaatggaggaaaacccagtgtatgccgcgcgaagtctgcattgacgtgggaaaggagttcggcgctgccaccaacaccttctttaagcctccctgcgtgtcggtgtatcgctgcgggggatgctgcaacagcgaaggccttcagtgcatgaacaccagcaccggatacctcagcaagactctcttcgaaatcactgtgccgctgtcacaaggcccgaagcctgtgaccatttccttcgccaaccacacctcctgtcggtgcatgagcaagctggatgtgtacagacaggtgcactccatcatccggagatcgttgcctgccaccctgccgcagtgccaagcggctaacaagacctgtcccaccaactacgtgtggaacaactatatgtgtcggtgcctggcacagcaggactttatcttctactccaacgtggaggacgactcgactaacggtttccacgacgtgtgcggacccaacaaggagctggatgaggatacttgtcagtgcgtgtgcaagggtggcctgcgcccgtcctcctgcggaccacataaggaactggacagggactcgtgccaatgcgtctgcaagaacaagctgttccctaactcctgcggggcgaaccgcgaattcgacgagaacacctgtcagtgtgtgtgcaagcggacttgcccgaggaatcagcctcttaaccccggaaaatgcgcctgcgaatgcacagagaacacccagaagtgcttcttgaaagggaagaagttccaccaccaaacctgttcatgctaccggcgcccatgtgccaaccggctgaagcactgcgacccgggattgagcttcagcgaggaggtctgcagatgcgtgccgtcatactggaagcgacctcatctgaattagcccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactgccccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtgcgcgcggccggcatggtcccagcctcctcgctggcgccggctgggcaacattccgaggggaccgtcccctcggtaatggcgaatgggacgtcgactgctaacaaagcccgaaaggaagctgagttggctgctgccaccgctgagcaataactagcataaccccttggggcctctaaacgggtcttgaggggttttttgctgaaaggaggaactatatgcgcgcagatctgtcatgatgatcattgcaattggatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 89 - DNA, 1302 bp, Artificial Sequence
  Additional transcription unit encoding codon optimized mVEGF-C with APMV gene end, intergenic and gene start sequences and Kozak sequence
gtcgacatttttaattaaaatagggtggggaaggtaccgccaccatgcacctgctgtgcttcctgagcctggcctgcagcctgctggccgccgccctgatccccagccccagagaggcccccgccaccgtggccgccttcgagagcggcctgggcttcagcgaggccgagcccgacggcggcgaggtgaaggccttcgagggcaaggacctggaggagcagctgagaagcgtgagcagcgtggacgagctgatgagcgtgctgtaccccgactactggaagatgtacaagtgccagctgagaaagggcggctggcagcagcccaccctgaacaccagaaccggcgacagcgtgaagttcgccgccgcccactacaacaccgagatcctgaagagcatcgacaacgagtggagaaagacccagtgcatgcccagagaggtgtgcatcgacgtgggcaaggagttcggcgccgccaccaacaccttcttcaagcccccctgcgtgagcgtgtacagatgcggcggctgctgcaacagcgagggcctgcagtgcatgaacaccagcaccggctacctgagcaagaccctgttcgagatcaccgtgcccctgagccagggccccaagcccgtgaccatcagcttcgccaaccacaccagctgcagatgcatgagcaagctggacgtgtacagacaggtgcacagcatcatcagaagaagcctgcccgccaccctgccccagtgccaggccgccaacaagacctgccccaccaactacgtgtggaacaactacatgtgcagatgcctggcccagcaggacttcatcttctacagcaacgtggaggacgacagcaccaacggcttccacgacgtgtgcggccccaacaaggagctggacgaggacacctgccagtgcgtgtgcaagggcggcctgagacccagcagctgcggcccccacaaggagctggacagagacagctgccagtgcgtgtgcaagaacaagctgttccccaacagctgcggcgccaacagagagttcgacgagaacacctgccagtgcgtgtgcaagagaacctgccccagaaaccagcccctgaaccccggcaagtgcgcctgcgagtgcaccgagaacacccagaagtgcttcctgaagggcaagaagttccaccaccagacctgcagctgctacagaagaccctgcgccaacagactgaagcactgcgaccccggcctgagcttcagcgaggaggtgtgcagatgcgtgcccagctactggaagagaccccacctgaactgaccgggtcgac

SEQ ID NO: 90 - DNA, 19845 bp, Artificial Sequence
  pRz-APMV4-mVEGF-C Rescue plasmid containing the full-length cDNA of APMV4 with an additional transcription unit encoding a codon-optimized mVEGF-C
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctttaatacgactcactatagggagattggtctgatgagtccgtgaggacgaaacggagtctagactccgtcacgaaaaagaagaataaaaggcagaagccttttaaaaggaaccctgggctgtcgtaggtgtgggaaggttgtattccgagtgcgcctccgaggcatctactctacacctatcacaatggctggtgtcttctcccagtatgagaggtttgtggacaatcaatcccaagtgtcaaggaaggatcatcggtccttagcaggaggatgccttaaagttaacatccctatgcttgtcactgcatctgaagaccccaccactcgttggcaactagcatgcttatctctaaggctcctgatctccaactcatcaaccagtgctatccgtcagggggcaatactgactctcatgtcattaccatcacaaaacatgagagcaacagcagctattgctggttccacaaatgcagctgttatcaacaccatggaagtcttaagtgtcaacgactggaccccatccttcgaccctaggagcggtctttctgaggaagatgctcaagttttcagagacatggcaagagatctgccccctcagttcacctctggatcacccttcacatcagcattggcggaggggttcactcctgaagatactcatgacctgatggaggccttgaccagtgtgctgatacagatctggatcctggtggctaaggccatgaccaacattgacggctctggggaggccaatgaaagacgtcttgcaaagtacatccaaaaaggacagcttaatcgtcagtttgcaattggtaatcctgcccgtctgataatccaacagacaatcaaaagctccttaactgtccgtaggttcttggtctctgagcttcgtgcgtcacgaggtgcagtaaaagaaggatccccttactatgcagctgttggggatatccacgcttacatctttaatgcgggattgacaccattcttgaccaccttaagatacgggataggcaccaagtacgccgctgttgcactcagtgtgttcgctgcagatattgcaaagttgaagagcctacttaccctgtaccaggacaagggtgtagaagctggatacatggcactccttgaggatccagactccatgcactttgcacctggaaacttcccacacatgtactcctatgcaatgggggtagcttcttaccatgatcctagcatgcgccaataccaatacgccaggaggttcctcagccgtcctttctacttactaggaagggacatggccgccaagaacacaggcacgctggatgagcaactggcgaaggaactgcaagtatcagagagagatcgcgccgcattatccgctgcgattcaatcagcgatggaggggggagagtccgacgacttcccactgtcgggatccatgccggctctctctgagaatgcgcaaccagttacccccagacctcaacagtcccagctctctcccccccaatcatcaaacatgccccaatcagcacccaggaccccagactatcaacccgactttgaactgtaggcttcatcaccgcaccaacaacagcccaagaagaccacccctccccccacacatctcacccagccacccataaagactcagtggcgcgccccagcatctccttcatttaattaaaaaccgaccaacagggtggggaaggagagtcattggctactgccaattgtgtgcagcaatggattttactgacattgatgctgtcaactcattgatcgaatcatcatcggcaatcatagactccatacagcatggagggctgcaaccagcgggcaccgtcggcctatcgcagatcccaaaagggataaccagcgcattaaccaaggcctgggaggctgaggcggcaactgccggtaatggggacacccaacacaaatctgacagtccggaggatcatcaggccaacgacacagattcccctgaagacacaggtactgaccagaccacccaggaggccaacatcgttgagacaccccaccccgaggtgctgtcagcagccaaagccagactcaagaggcccaaagcagggagggacacccgcgacaactcccctgcgcaacccgatcatcttttaaaggggggcctcctgagcccacaaccagcagcatcatgggtgcaaaatccacccagtcatggaggtcccggcaccgccgatccccgcccatcacaaactcaggatcattcccccaccggagagaaatggcgattgtcaccgacaaagcaaccggagacattgaactggtggagtggtgcaacccggggtgcacagcagtccgaattgaacccaccagactcgactgtgtatgcggacactgccccaccatctgtagcctctgcatgtatgacgactgatcaggtacaactactaatgaaggaggttgctgacataaaatcactccttcaggcgttagtgaggaacctcgctgtcttgccccaattgaggaatgaggttgcagcaatcagaacatcacaggccatgatagaggggacactcaattcgatcaagattcttgaccctgggaattatcaggaatcatcactaaacagttggttcaaacctcgccaagatcacactgttgttgtgtctggaccagggaatccattggccatgccaaccccagtccaagacaacaccatattcctggacgagctagccagacctcatcctagtgtggtcaatccatccccgcccatcaccaacaccaatgttgaccttggcccacagaagcaggctgcaatagcctatatctccgctaaatgcaaggatccggggaaacgagatcagctatcaaggctcattgagcgagcaaccaccccaagtgagatcaacaaagttaaaagacaagcccttgggctctagatcactcgatcacccctcatggtgatcacaacaataatcagaacccttccgaaccacatgaccaacccagcccaccgcccacaccgtccgtcgacatttttaattaaaatagggtggggaaggtaccgccaccatgcacctgctgtgcttcctgagcctggcctgcagcctgctggccgccgccctgatccccagccccagagaggcccccgccaccgtggccgccttcgagagcggcctgggcttcagcgaggccgagcccgacggcggcgaggtgaaggccttcgagggcaaggacctggaggagcagctgagaagcgtgagcagcgtggacgagctgatgagcgtgctgtaccccgactactggaagatgtacaagtgccagctgagaaagggcggctggcagcagcccaccctgaacaccagaaccggcgacagcgtgaagttcgccgccgcccactacaacaccgagatcctgaagagcatcgacaacgagtggagaaagacccagtgcatgcccagagaggtgtgcatcgacgtgggcaaggagttcggcgccgccaccaacaccttcttcaagcccccctgcgtgagcgtgtacagatgcggcggctgctgcaacagcgagggcctgcagtgcatgaacaccagcaccggctacctgagcaagaccctgttcgagatcaccgtgcccctgagccagggccccaagcccgtgaccatcagcttcgccaaccacaccagctgcagatgcatgagcaagctggacgtgtacagacaggtgcacagcatcatcagaagaagcctgcccgccaccctgccccagtgccaggccgccaacaagacctgccccaccaactacgtgtggaacaactacatgtgcagatgcctggcccagcaggacttcatcttctacagcaacgtggaggacgacagcaccaacggcttccacgacgtgtgcggccccaacaaggagctggacgaggacacctgccagtgcgtgtgcaagggcggcctgagacccagcagctgcggcccccacaaggagctggacagagacagctgccagtgcgtgtgcaagaacaagctgttccccaacagctgcggcgccaacagagagttcgacgagaacacctgccagtgcgtgtgcaagagaacctgccccagaaaccagcccctgaaccccggcaagtgcgcctgcgagtgcaccgagaacacccagaagtgcttcctgaagggcaagaagttccaccaccagacctgcagctgctacagaagaccctgcgccaacagactgaagcactgcgaccccggcctgagcttcagcgaggaggtgtgcagatgcgtgcccagctactggaagagaccccacctgaactgaccgggtcgacatcccttgccaaacatcctgccgtagctgatttattcaaaagagctcatttgatatgacctggtaatcataaaatagggtggggaaggtgctttgcctgtaagggggctccctcatcttcagacacgtgcccgccatctcaccaacagtgcaatggcagacatggacacggtgtatatcaatctgatggcagatgacccaacccaccaaaaagaactgctgtcctttcctctcatccctgtgaccggtcctgacgggaagaaggaactccaacaccagatccggacccaatccttgctcgcctcagacaaacaaactgaacggttcatcttcctcaacacttacggattcatctatgacaccacaccggacaagacaactttttccaccccagagcatattaatcagcctaagaggacgacggtgagtgccgcgatgatgaccattggcctggttcccgccaatatacccctgaacgaactaacggctactgtgttcagccttaaagtaagagtgaggaaaagtgcgaggtatcgggaagtggtctggtatcaatgcaatccagtaccggccctgcttgcagccaccaggtttggtcgccaaggaggtctcgagtcgagcactggagtcagtgtaaaggctcccgagaagatagattgtgagaaggattatacctactacccttatttcttatctgtgtgctacatcgccacctccaacctgttcaaggtaccgaggatggttgctaatgcaaccaacagtcaattataccaccttaccatgcaggtcacatttgcctttccaaaaaacatccctccagccaaccagaaactcctgacacaggtggatgagggattcgagggcactgtggattgccattttgggaacatgctgaaaaaggatcggaaagggaacatgaggacactgtcccaggcggcagataaggtcagacgaatgaatattcttgttggtatctttgacttgcatgggccaacgctcttcctggagtataccgggaaactgacaaaggctctgctagggttcatgtccaccagccgaacagcaatcatccccatatctcagctcaatcccatgctgagtcaactcatgtggagcagtgatgcccagatagtaaagttaagggttgtcataactacatccaaacgcggcccgtgcgggggtgagcaggagtatgtgctggatcccaaattcacagttaagaaagaaaaggctcgactcaaccctttcaagaaggcagcctaatgatttaatccgcaagatcccagaaatcagaccactctatactatccactgatcactggaaatgtaatcctgcaggtgatgaatctgtgaagaatcaattaaaaaaccggatccttattagggtggggaagtagttgattgggtgtctaaacaaaagcatttcttcacacctccccgccacgaaacaaccacaatgaggctatcaaacacaatcttgaccttgattctcatcatacttaccggctatttgataggtgtccactccaccgatgtgaatgagaaaccaaagtccgaagggattaggggtgatcttacaccaggtgcgggtattttcgtaactcaagtccgacagctccagatctaccaacagtctgggtaccatgatcttgtcatcagattgttacctcttctaccaacagagcttaatgattgtcaaagggaagttgtcacagagtacaataacactgtatcacagctgttgcagcctatcaaaaccaacctggatactttgttggcagatggtagcacaagggatgttgatatacagccgcgattcattggggcaataatagccacaggtgccctggctgtagcaacggtagctgaggtaactgcagctcaagcactatctcagtcaaaaacgaatgctcaaaatattctcaagttgagagatagtattcaggccaccaaccaagcagtttttgaaatttcacagggactcgaagcaactgcaaccgtgctatcaaaactgcaaactgagctcaatgagaatatcatcccaagtctgaacaacttgtcctgtgctgccatggggaatcgccttggtgtatcactctcactctatttgaccttaatgaccactctatttggggaccagatcacaaacccagtgctgacgccaatctcttacagcaccctatcggcaatggcgggtggtcacattggtccagtgatgagtaagatattagccggatctgtcacaagtcagttgggggcagaacaactgattgctagtggcttaatacagtcacaggtagtaggttatgattcccagtatcagctgttggttatcagggtcaaccttgtacggattcaggaagtccagaatactagggttgtatcactaagaacactagcagtcaatagggatggtggactttacagagcccaggtgccacccgaggtagttgagcgatctggcattgcagagcggttttatgcagatgattgtgttctaactacaactgattacatctgctcatcgatccgatcttctcggcttaatccagagttagtcaagtgtctcagtggggcacttgattcatgcacatttgagagggaaagtgcattactgtcaactcccttctttgtatacaacaaggcagtcgtcgcaaattgtaaagcagcgacatgtagatgtaataaaccgccatctatcattgcccaatactctgcatcagctctagtaaccatcaccaccgacacttgtgctgaccttgaaattgagggttatcgtttcaacatacagactgaatccaactcatgggttgcaccaaacttcacggtctcaacctcacaaatagtatcggttgatccaatagacatatcctctgacattgccaaaattaacaattctatcgaggctgcgcgagagcagctggaactgagcaaccagatcctttcccgaatcaacccacggattgtgaacgacgaatcactaatagctattatcgtgacaattgttgtgcttagtctccttgtaattggtcttattattgttctcggtgtgatgtacaagaatcttaagaaagtccaacgagctcaagctgctatgatgatgcagcaaatgagctcatcacagcctgtgaccaccaaattggggacacccttctaggtgaataatcatatcaatccattcaataatgagcgggacataccaatcaccaacgactgtgtcacaaggccggttaggaatgcaccggatctctctccttcctttttaattaaaaacgatcgaactgagggtgagggggggggtgtgcatggtagggtggggaaggtagccaattcctgcccattgggccgaccgtaccaagagaagtcaacagaagtatagatgcagggcgacatggagggtagccgtgataacctcacagtagatgatgaattaaagacaacatggaggttagcttatagagttgtatccctcctattgatggtgagtgccttgataatctctatagtaatcctgacgagagataacagccaaagcataatcacggcgatcaaccagtcgtatgacgcagactcaaagtggcaaacagggatagaagggaaaatcacctcaatcatgactgatacgctcgataccaggaatgcagctcttctccacattccactccagctcaatacacttgaggcaaacctgttgtccgccctcggaggtaacacgggaattggccccggagatctagagcactgtcgttatccggttcatgactccgcttacctgcatggagtcaatcgattactcatcaatcaaacagctgactacacagcagaaggccccctggatcatgtgaacttcattccggcaccagttacgactactggatgcacaaggatcccatccttttctgtatcatcatccatttggtgctatacacacaatgtgattgaaacaggttgcaatgaccactcaggtagtaatcaatatatcagtatgggggtgattaagagggctggcaacggcttaccttacttctcaacagtcgtgagtaagtatctgaccgatgggttgaatagaaaaagctgttccgtagctgccggatccgggcattgttacctcctttgtagcctagtgtcagagcccgaacctgatgactatgtgtcaccagatcccacaccgatgaggttaggggtgctaacaagggatgggtcttacactgaacaggtggtacccgaaagaatatttaagaacatatggagcgcaaactaccctggggtagggtcaggtgctatagtaggaaataaggtgttattcccattttacggcggagtgaagaatggatcaacccctgaggtgatgaataggggaagatattactacatccaggatccaaatgactattgccctgacccgctgcaagatcagatcttaagggcagaacaatcgtattatcctactcgatttggtaggaggatggtaatgcagggagtcctaacatgtccagtatccaacaattcaacaatagccagccaatgccaatcttactatttcaacaactcattaggattcatcggggcggaatctaggatctattacctcaatggtaacatttacctttatcaaagaagctcgagctggtggcctcacccccaaatttacctacttgattccaggattgcaagtccgggtacgcagaacattgactcaggcgttaacctcaagatgttaaatgttactgtcattacacgaccatcatctggcttttgtaatagtcagtcaagatgccctaatgactgcttattcggggtttattcagatgtctggcctcttagccttacctcagacagcatatttgcatttacaatgtacttacaagggaagacgacacgtattgacccagcttgggcgctattctccaatcatgtaattgggcatgaggctcgtttgttcaacaaggaggttagtgctgcttattctaccaccacttgtttttcggacaccatccaaaaccaggtgtattgtctgagtatacttgaagtcagaagtgagctcttgggggcattcaagatagtgccattcctctatcgtgtcttataggcacctgcttggtcaagaaccctgagcggccgtaaaattaacacttgatcttccttaaaaacacctatctaaattactgtctgagatccctgattagttaccctttcaatcaatcaattaatttttaattaaaaacggaaaaatgggcctagttccaaggaaaggatgggacccattagggtggggaaggattactttgttccttgactcgcacccacgtacacccaatcccattcctgtcaagaaggaacccttcccaaactcaccttgcaatgtccaatcaggcagctgagattatactacccaccttccatctggaatcacccttgatcgagaataagtgcttctactacatgcaattacttggtctcgtgttaccacatgatcactggagatggagggcattcgtcaattttacagtggatcaagcacaccttaaaaatcgtaatccccgcttaatggcccacatcgatcacactaaggatagactaagggctcatggtgtcttgggtttccaccagactcagacaagtgagagccgtttccgtgtcttgctccatcctgaaactttaccttggctatcagcaatgggaggatgcatcaaccaggttcccaaggcatggcggaacactctgaaatctatcgagcacagtgtgaagcaggaggcgactcaactgaagttactcatggaaaaaacctcactaaagctaacaggagtatcttacttattctccaattgcaatcccgggaaaactgcagcgggaactatgcccgtactaagtgagatggcatcagaactcttgtcaaatcccatctcccaattccaatcaacatgggggtgtgctgcttcagggtggcaccatgtagtcagcatcatgaggctccaacagtatcaaagaaggacaggtaaggaagagaaagcaatcactgaagttcagtatggctcggacacctgtctcattaatgcagactacaccgtcgttttttccgcacaggaccgtgtcatagcagtcttgcctttcgatgttgtcctcatgatgcaagacctgcttgaatcccgacggaatgtcttgttctgtgcccgctttatgtatcccagaagccaactacatgagaggataagtacaatactggcccttggagaccaactcgggagaaaagcaccccaagtcctgtatgatttcgtagctaccctcgaatcatttgcatacgctgctgtccaacttcatgacaacaaccctatctacggtggggctttctttgagttcaatatccaagaactggaagctattttgtcccctgcacttaataaggatcaagtcaacttctacataagtcaagttgtctcagcatacagtaaccttcccccatctgaatcagcagaattgctatgcttactacgcctgtggggtcatcccttgctaaacagtcttgatgcagcaaagaaagtcagagaatctatgtgtgctgggaaggttcttgattataatgctattcgactagttttgtctttttatcatacgttattaatcaatgggtatcggaagaaacataagggtcgctggccaaatgtgaatcaacattcactactcaacccgatagtgaagcagctttactttgatcaggaggagatcccacactctgttgcccttgagcactatttagatatctcgatgatagaatttgagaagacttttgaagtggaactatctgatagtctaagcatctttctgaaggataagtcgatagctttggataaacaagaatggcacagtggttttgtctcagaagtgactccaaagcacctacgaatgtctcgtcatgatcgcaagtctaccaataggctattgttagcctttattaactcccctgaattcgatgttaaggaagagcttaaatatttgactacaggtgagtatgccactgacccaaatttcaatgtctcttactcactgaaagagaaggaagttaagaaagaagggcgcattttcgcaaagatgtcacagaaaatgagagcatgccaggttatttgtgaagagttactagcacatcatgtggctcctttgtttaaagagaatggtgttacacaatcggagctatccctgacaaagaatttgttggctattagccaactgagttacaactcgatggccgctaaggtgcgattgctgaggccaggggacaagttcaccgctgcacactatatgaccacagacctaaaaaagtactgccttaactggcggcaccagtcagtcaaattgttcgccagaagcctggatcgactatttgggttagaccatgctttttcttggatacacgtccgtctcaccaatagcactatgtacgttgctgacccattcaatccaccagactcagatgcatgcacaaatttagacgacaataagaacactgggatttttattataagtgctcgaggtggtatagaaggccttcaacagaaactatggactggcatatcaattgcaatcgcccaggcggcagcagccctcgagggcttacgaattgctgccactttgcagggggataaccaggttttagcgattacgaaagaattcatgaccccagtctcggaggatgtaatccacgagcagctatctgaagcgatgtcgcgatacaagaggactttcacataccttaattatttaatggggcaccaattgaaggataaagaaaccatccaatccagtgacttcttcgtttactccaaaaggatcttcttcaatgggtcaatcctaagtcaatgcctcaagaacttcagtaaactcactaccaatgccactacccttgctgagaacactgtagccggctgcagtgacatctcctcatgcatagcccgttgtgtggaaaacgggttgcctaaggatgctgcatatgttcagaatataatcatgactcggcttcaactgttgctagatcactactattctatgcatggtggcataaactcagagttagagcagccaactctaagtatccctgtccgaaacgcaacctatttaccatctcaattaggcggttacaatcatttgaatatgacccgactattctgtcgcaatatcggtgacccgcttactagttcttgggcagagtcaaaaagactaatggatgttggccttctcagtcgtaagttcttagaggggatattatggagacccccgggaagtgggacattttcaacactcatgcttgatccgttcgcacttaacattgattacttaaggccaccagagacaataatccgaaaacacacccaaaaagtcttgttgcaggattgtcctaatcctctattagcaggtgtagttgacccgaactacaaccaggaattagaattattagctcagttcctgcttgatcgggaaaccgttattcccagggctgcccatgccatctttgaactgtctgtcttgggaaggaaaaaacatatacaaggattggttgatactacaaaaacaattattcagtgctcattagaaagacagccactgtcctggaggaaagttgagaacattgtaacctacaatgcgcagtatttcctcggggccacccagcaggttgacaccaatatctcagaaaggcagtgggtgatgccaggtaatttcaagaagcttgtatctcttgacgattgctcagtcacgttgtccactgtgtcacggcgcatttcttgggccaatctacttaactggagggctatagatggtttggaaactccagatgtgatagagagtattgatggccgccttgtgcaatcatccaatcaatgcggcctatgtaatcaaggattgggctcctactcctggttcttcttgccctccgggtgtgtgttcgaccgtccacaagattctcgagtggttccaaagatgccatacgtgggatccaaaacggatgagagacagactgcgtcagtgcaggctatacagggatccacatgtcaccttagagcagcattgagacttgtatcactctacctttgggcctatggagattctgacatatcatggctagaagccgcgacattggctcaaacacggtgcaatatttctcttgatgacctgcggatcctgagccctcttccttcctcggcaaatttacaccacagattgaatgacggggtaacacaagtgaaattcatgcccgccacatcgagccgggtgtcaaagttcgtccaaatttgcaatgacaaccagaatcttatccgtgatgatgggagtgttgattccaatatgatttatcagcaggttatgatattagggcttggagagattgaatgtttgttagctgacccaatcgatacaaacccagaacaactgattcttcacctacactctgataattcttgctgtctccgggagatgccaacgaccggttttgtacctgctttaggattgaccccatgcttaactgtcccaaagcacaatccgtatatttatgatgatagcccaatacccggtgatttggatcagaggctcattcaaaccaaattctttatgggttctgacaatctagataatcttgatatctaccagcagcgagctttactgagtcggtgtgtggcttatgacattatccaatcagtattcgcttgcgatgcaccagtatctcagaagaatgatgcaatccttcacactgactaccatgaaaattggatctcagagttccgatggggtgaccctcgcataatccaagtaacagcaggttacgagttaattctgttccttgcataccagctttattatctcagagtgaggggtgaccgtgcaatcctgtgttatattgataggatactcaacaggatggtatcttccaatctaggcagtctcatccagacgctctctcatccggagattaggaggagattttcattgagtgatcaagggttccttgtcgaaagggagctagagccaggtaagccactggtaaaacaagcggttatgttcctaagggactcagtccgctgcgctttagcaactatcaaggcaggaattgagcctgagatctcccgaggtggctgtacccaggatgagctgagctttacccttaagcacttactatgtcggcgtctctgtataattgctctcatgcattcggaagcaaagaacttggtcaaagttagaaaccttccagtagaggaaaaaaccgccttactataccagatgttgatcactgaggccaatgccaggagatcagggtctgctagtatcatcataagcttagtttcagcaccccagtgggacattcatacaccagcgttgtattttgtatcaaagaaaatgctggggatgctcaaaaggtcaaccacacccttggatataagtgacctttctgagagccagaacctcacaccaacagaattgaatgatgttcctggtcacatggcagaggaatttccctgtttgtttagcagttataacgctacatatgaagacacaattacttacaatccaatgactgaaaaactcgcagtgcacttggacaatggttccaccccttccagagcgcttggtcgtcactacatcctgcgaccccttgggctttactcgtctgcatggtaccggtctgcagcactattagcgtcaggggccctcagtgggttgcctgaggggtcaagcctgtacttgggagaggggtatgggaccaccatgactctacttgagcccgttgtcaagtcctcaactgtttactaccatacattgtttgacccaacccggaatccttcacagcggaactacaaaccagaaccgcgggtattcactgattccatttggtacaaggatgatttcacacgaccacctggtggcattgtaaatctatggggtgaagacgtacgtcagagtgatattacacagaaagacacggttaatttcatattatctcgggtcccgccaaaatcactcaaattgatacacgttgatattgagttctccccagactctgatgtacggacgctactatctggctattcccattgtgcactattggcctactggctactgcaacctggagggcgatttgcggttagagttttcttaagtgaccatatcatagtcaacttggtcactgccattctgtccgcttttgactctaatctggtgtgcattgcgtcaggattgacacacaaggatgatggggcaggttatatttgtgcaaagaagcttgcaaatgttgaggcttcaagaattgagtattacttgaggatggtccacggctgtgttgactcattaaaaattcctcatcaattaggaatcattaaatgggctgagggtgaagtgtcccgacttaccaaaaaggcggatgatgaaataaactggcggttaggtgatccagttaccagatcatttgatccggtttctgagctaataattgcgcgaacagggggatcagtattaatggaatacgggacttttactaacctcaggtgtgcgaacttggcagatacatataaacttttggcttcaattgtagagaccaccttaatggaaataagggttgagcaagatcagttggaagatgattcgaggagacaaatccaggtagtccctgcttttaatacaagatccgggggaaggatccgtacattgattgagtgtgctcagctgcaggtcatagatgttatctgtgtgaacatagatcacctctttcccaaacaccgacatgctcttgtcacacaacttacttaccagtcagtgtgccttggggacttgattgaaggcccccaaattaagacatatctaagggccaggaagtggatccaacgtaggggactcaatgagacaattaaccatatcatcactggacaagtgtcgcggaataaggcaagggattttttcaagaggcgcctgaagttggttggcttttcgctctgtggcggttggggctacctctcactttagctgcttagattgttgattattatgaataatcggagtcgaaatcgtaaatagaaagacataaaattgcaaataagcaatgatcgtattaatatttaataaaaaatatgtcttttatttcttgcgcgcggccggcatggtcccagcctcctcgctggcgccggctgggcaacattccgaggggaccgtcccctcggtaatggcgaatgggacgtcgactgctaacaaagcccgaaaggaagctgagttggctgctgccaccgctgagcaataactagcataaccccttggggcctctaaacgggtcttgaggggttttttgctgaaaggaggaactatatgcgcgcagatctgtcatgatgatcattgcaattggatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 91 - protein, 6 aa, Artificial Sequence
  Cleavage Site Modification
  MISC_FEATURE (6)..(6): X is S or F
HNRTKX

SEQ ID NO: 92 - protein, 6 aa, Artificial Sequence
  Cleavage Site Modification
  MISC_FEATURE (6)..(6): X is S or F
HNKTKX

SEQ ID NO: 93 - protein, 6 aa, Artificial Sequence
  Cleavage Site Modification
  MISC_FEATURE (6)..(6): X is S or F
HNRMKX

SEQ ID NO: 94 - protein, 7 aa, Artificial Sequence
  Cleavage Site Modification
  MISC_FEATURE (6)..(6): X is S or F
HNKMKXI

SEQ ID NO: 95 - protein, 7 aa, Artificial Sequence
  Cleavage Site Modification
  MISC_FEATURE (6)..(6): X is R or F
HNRTKXI

SEQ ID NO: 96 - DNA, 2069 bp, Homo sapiens
  Human vascular endothelial growth factor-D (VEGF-D) (GenBank No. NM_004469.5; Uni-Prot. 043915)
aagacacatgcttctgcaagcttccatgaaggttgtgcaaaaaagtttcaatccagagttgggttccagctttctgtagctgtaagcattggtggccacaccacctccttacaaagcaactagaacctgcggcatacattggagagatttttttaattttctggacatgaagtaaatttagagtgctttctaatttcaggtagaagacatgtccaccttctgattatttttggagaacattttgatttttttcatctctctctccccacccctaagattgtgcaaaaaaagcgtaccttgcctaattgaaataatttcattggattttgatcagaactgattatttggttttctgtgtgaagttttgaggtttcaaactttccttctggagaatgccttttgaaacaattttctctagctgcctgatgtcaactgcttagtaatcagtggatattgaaatattcaaaatgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttgattcagcgttccaagttccccatccctgtcatttttaacagcatgctgctttgccaagttgctgtcactgtttttttcccaggtgttaaaaaaaaaatccattttacacagcaccacagtgaatccagaccaaccttccattcacaccagctaaggagtccctggttcattgatggatgtcttctagctgcagatgcctctgcgcaccaaggaatggagaggaggggacccatgtaatccttttgtttagttttgtttttgttttttggtgaatgagaaaggtgtgctggtcatggaatggcaggtgtcatatgactgattactcagagcagatgaggaaaactgtagtctctgagtcctttgctaatcgcaactcttgtgaattattctgattcttttttatgcagaatttgattcgtatgatcagtactgactttctgattactgtccagcttatagtcttccagtttaatgaactaccatctgatgtttcatatttaagtgtatttaaagaaaataaacaccattattcaagcca

SEQ ID NO: 97 - DNA, 2322 bp, Canis lupus
  Canine VEGF-D, Canis lupus familiaris (GenBank No. XM_548869.5)
ttttctgtgtgtccgtggcagtcgatgtgtgaacatctgaggtcccttcctgagcattgcgatttccatgcaacattcatgcctgtgtgctggggtttcacgttacaggttatctgcattaaaataacagcagtcctgatggtttgagtcagttttcaaaactgccctgctattggtagggacgcgacaggattacagccaagacttccctgcattttctgccaaagtctctgtcagatttaagacacatgcttccgcaaccttccatgagggttgtaaaaaaagtctgaatccagaattgggttccagccttctgtggctgcaaacattggtggccacaccacctccttacaaagcaactagaacctggggcagagggtggagagatttttttttttaatttgctggacatgaaatgaatttagagtgctttttcgtgtcaagtggaagtcatgtccacctcctgattatttttggagcatgagtgcatttaattttttttcatctctctccccgcataagattgagcaaaaacgttccttgactaattgaagtcatttcattggattttgatcacaactgattatttgggttttttccatgtgaagtcttggggtttcgaactttccttctggagaatgccttttgaaacagttttctctagctgcctgatgtcaactgcttggtaatcggtggacattaaaatactcaaaatgtacagacagtgggccgtggtgaatgttttcatgatgtcttatctacagttggtgcacagctccagttatgagcatggaccagtgaagcgggcatctcggtcaacgttagagcggtctgaacagcagattagggcagcttctggtttggaagaactgctgcggatcacacacttcgaggactggaagctctggagatgccgactgaagctcaaaagtttgaccagcacagactctcgctcagcatcccatcgggccaccaggtttgcggcaactttctatgacattgaaaccctaaaagtcatagacgaggagtggcagcggacgcagtgcagcccccgggagacgtgcgtggaggtggccagcgagctggggaggagcaccgacacgttcttcaagccgccctgcgtgaacgtgttccgctgtggcggctgctgcaacgaggagagcctcgtctgtatgaacacgagcacctcctacgtctccaaacagctctttgagatatcagtgcctttgacttcagtacctgaattagtgcctgttaaggtggccaaccatacaggttgtaagtgcctgccaacggctccccgccatccatactccattatcagaagatccatccagatcccagaagaagatcactgttcccattccaagcaactctgtcctgttgacatgctatgggatagcgacaaatgtaaatgtgttttacaggaggagaatccactcgttggaatggaagaccactctcacctccaggaactggctctctgcgggccgcacatgaagtttgacgacgatcgttgcgagtgtgtctgtaaaacaccgtgtcccagagatctcatccagcacccagaaaactgcagttgcatggagtgcagagagagcctggagagctgctgccagaagcacaagatatttcacgcagacacctgcagctgtgaggacagatgtccctttcacaccagaacatgtgcgcatggaagaccagcatgtgcaaagcactgccgctttccgaaggagaaaagggctgcctatgggttccatggtcaagaaaatccttgactcaacttggttcctgagttccccatccctaacattttaaacagcatgctgctttgccaagttgctgtcactgattgtttttttttccccacgtacaagaaaaaaaaatctgttttacccagtcccacaatgaattcagaccacccttccattacacaccagctgaggcttccctggttcactgacagatgactgccaactgaagatgcccctgcacagcaggatggagaggagggaacctgtagcagccccctcccttttttttttggtgaatgttaaaggtctgatcatcctagaatcacaggggccataaaattgattactcaaagccaacaaggcaattttatagtctccaagtccttcgctaatgcagctgtcttgtgaattcttctgactctttattatgcagattttgatttgtatgatcagcactgattttctgattactgtccagcttgtagttttgagtttactgaactactgtctgttgtttcatatttaagtgtatttaaagaaaataaacaccattattcaagccgtggaa

SEQ ID NO: 98 - DNA, 2321 bp, Canis lupus
  Canine VEGF-D, Canis lupus dingo (GenBank No. XM_025437083)
ttttctgtgtgtccgtggcagtcgatgtgtgaacatctgaggtcccttcctgagcattgcgatttccatgcaacattcatgcctgtgtgctggggtttcacgttacaggttatctgcattaaaataacagcagtcctgatggtttgagtcagttttcaaaactgccctgctattggtagggacgcgacaggattacagccaagacttccctgcattttctgccaaagtctctgtcagatttaagacacatgcttccgcaaccttccatgagggttgtaaaaaaagtctgaatccagaattgggttccagccttctgtggctgcaaacattggtggccacaccacctccttacaaagcaactagaacctggggcagagggtggagagatttttttttttaatttgctggacatgaaatgaatttagagtgctttttcgtgtcaagtggaagtcatgtccacctcctgattatttttggagcatgagtgcatttaattttttttcatctctctccccgcataagattgagcaaaaacgttccttgactaattgaagtcatttcattggattttgatcacaactgattatttgggttttttccatgtgaagtcttggggtttcgaactttccttctggagaatgccttttgaaacagttttctctagctgcctgatgtcaactgcttggtaatcggtggacattaaaatactcaaaatgtacagacagtgggccgtggtgaatgttttcatgatgtcttatctacagttggtgcacagctccagttatgagcatggaccagtgaagcgggcatctcggtcaacgttagagcggtctgaacagcagattagggcagcttctggtttggaagaactgctgcggatcacacacttcgaggactggaagctctggagatgccgactgaagctcaaaagtttgaccagcacagactctcgctcagcatcccatcgggccaccaggtttgcggcaactttctatgacattgaaaccctaaaagtcatagacgaggagtggcagcggacgcagtgcagcccccgggagacgtgcgtggaggtggccagcgagctggggaggagcaccgacacgttcttcaagccgccctgcgtgaacgtgttccgctgtggcggctgctgcaacgaggagagcctcgtctgtatgaacacgagcacctcctacgtctccaaacagctctttgagatatcagtgcctttgacttcagtacctgaattagtgcctgttaaggtggccaaccatacaggttgtaagtgcctgccaacggctccccgccatccatactccattatcagaagatccatccagatcccagaagaagatcactgttcccattccaagcaactctgtcctgttgacatgctatgggatagcgacaaatgtaaatgtgttttacaggaggagaatccactcgttggaatggaagaccactctcacctccaggaactggctctctgcgggccgcacatgaagtttgacgacgatcgttgcgagtgtgtctgtaaaacaccgtgtcccagagatctcatccagcacccagaaaactgcagttgcatggagtgcagagagagcctggagagctgctgccagaagcacaagatatttcacgcagacacctgcagctgtgaggacagatgtccctttcacaccagaacatgtgcgcatggaagaccagcatgtgcaaagcactgccgctttccgaaggagaaaagggctgcctatgggttccatggtcaagaaaatccttgactcaacttggttcctgagttccccatccctaacattttaaacagcatgctgctttgccaagttgctgtcactgattgtttttttttccccacgtacaagaaaaaaaaatctgttttacccagtcccacaatgaattcagaccacccttccattacacaccagctgaggcttccctggttcactgacagatgactgccaactgaagatgcccctgcacagcaggatggagaggagggaacctgtagcagccccctccctttttttttggtgaatgttaaaggtctgatcatcctagaatcacaggggccataaaattgattactcaaagccaacaaggcaattttatagtctccaagtccttcgctaatgcagctgtcttgtgaattcttctgactctttattatgcagattttgatttgtatgatcagcactgattttctgattactgtccagcttgtagttttgagtttactgaactactgtctgttgtttcatatttaagtgtatttaaagaaaataaacaccattattcaagccgtggaa

SEQ ID NO: 99 - protein, 354 aa, Canis lupus
  Canis Lupus Dingo VEGF-D (GenBank Accession No. XP_025292868.1)
MYRQWAVVNVFMMSYLQLVHSSSYEHGPVKRASRSTLERSEQQIRAASGLEELLRITHFEDWKLWRCRLKLKSLTSTDSRSASHRATRFAATFYDIETLKVIDEEWQRTQCSPRETCVEVASELGRSTDTFFKPPCVNVFRCGGCCNEESLVCMNTSTSYVSKQLFEISVPLTSVPELVPVKVANHTGCKCLPTAPRHPYSIIRRSIQIPEEDHCSHSKQLCPVDMLWDSDKCKCVLQEENPLVGMEDHSHLQELALCGPHMKFDDDRCECVCKTPCPRDLIQHPENCSCMECRESLESCCQKHKIFHADTCSCEDRCPFHTRTCAHGRPACAKHCRFPKEKRAAYGFHGQENP

SEQ ID NO: 100 - protein, 354 aa, Canis lupus; identical to SEQ ID NO: 99
  Canis Lupus Familiaris VEGF-D (GenBank Accession No. XP_548869.2)

SEQ ID NO: 101 - protein, 117 aa, Homo sapiens
  Human dNdC VEGF-D (mature, processed VEGF-D)
FAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRR

SEQ ID NO: 102 - protein, 149 aa, Homo sapiens
  Human VEGF-D C-terminal propeptide
SIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 103 - protein, 67 aa, Homo sapiens
  Human VEGF-D N-terminal propeptide
SSNEHGPVKRSSQSTLERSEQQIRAASSLEELLRITHSEDWKLWRCRLRLKSFTSMDSRSASHRSTR

SEQ ID NO: 104 - protein, 354 aa, Homo sapiens
  Human VEGF-D (UniProt O43915)
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 105 - protein, 21 aa, Homo sapiens
  Human VEGF-D signal peptide
MYREWVVVNVFMMLYVQLVQG